(19) United States
(12) Patent Application Publication
(10) Pub. No.: US 2007/0010671 A1
(43) Pub. Date: Jan. 11, 2007

(54) Novel quinazoline derivatives and methods of treatment related to the use thereof

(76) Inventors: Yoshinori Sekiguchi, Tokyo (JP); Kosuke Kanuma, Tokyo (JP); Katsunori Omodera, Tokyo (JP); Tsuyoshi Busujima, Tokyo (JP); Thuy-Anh Tran, San Diego, CA (US); Sangdon Han, San Diego, CA (US); Martin Casper, San Diego, CA (US); Bryan A. Kramer, San Diego, CA (US)

Correspondence Address: SUGHRUE MION, PLLC, 2100 PENNSYLVANIA AVENUE, N.W., SUITE 800, WASHINGTON, DC, 20037, US

(21) Appl. No.: 10/551,431

(22) PCT Filed: Mar. 30, 2004
(86) PCT No.: PCT/JP04/04554

§ 371 Date: Aug. 24, 2006

Related U.S. Application Data
(60) Provisional application No. 60/458,424, filed on Mar. 31, 2003.

(51) Int. Cl. C07D 403/02; C07D 239/94

(52) U.S. Cl. 544/284; 544/293

(57) ABSTRACT

The present invention relates to novel compounds of Formula (1): 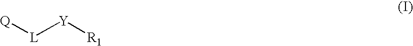 which act as MCH receptor antagonists. These compositions are useful in pharmaceutical compositions whose use includes prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction.

### FIELD OF THE INVENTION

The present invention relates to compounds which act as antagonists for MCH receptors and to the use of these compounds in pharmaceutical compositions.

### BACKGROUND OF THE INVENTION

Melanin Concentrating Hormone (MCH), a cyclic peptide, has been identified as the endogenous ligand of the orphan G-protein coupled receptor SLC-1. See, for example, Shimomura et al., Biochem. Biophys. Res. Commun. 261, 622-26 (1999). Studies have indicated that MCH acts as a neurotransmitter/neuromodulator to alter a number of behavioral responses such as feeding habits. For example, injection of MCH into rats has been reported to increase their consumption of food. Reports indicate that genetically engineered mice which lack MCH show lower body weight and increased metabolism. See Saito et al., TEM, vol. 11, 299 (2000). As such, the literature suggests that discovery of MCH antagonists that interact with SCL-1 expressing cells will be useful in developing obesity treatments. See Shimomura et al., Biochem. Biophys. Res. Commun. 261, 622-26 (1999).

G protein-coupled receptors (GPCRs) share a common structural motif. All these receptors have seven sequences of between 22 to 2z4 hydrophobic amino acids that form seven alpha helices, each of which spans the membrane. The fourth and fifth transmembrane helices are joined on the extracellular side of the membrane by a strand of amino acids that forms a relatively large loop. Another larger loop, composed primarily of hydrophilic amino acids, joins transmembrane helices five and six on the intracellular side of the membrane. The carboxy terminus of the receptor lies intracellularly and the amino terminus lies in the extracellular space. It is thought that the loop joining helices five and six, as well as the carboxy terminus, interact with the G protein. Currently, Gq, Gs, Gi, and Go are G proteins that have been identified as possible proteins that interact with the receptor.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Changing the receptor conformation to the active state allows linkage to the transduction pathwvay and produces a biological response.

A receptor may be stabilized in an active state by an endogenous ligand or an exogenous agonist ligand. Recent discoveries, including but not exclusively limited to, modifications to the amino acid sequence of the receptor, provide alternative mechanisms other than ligands to stabilize the active state conformation. These approaches effectively stabilize the receptor in an active state by simulating the effect of a ligand binding to the receptor. Stabilization by such ligand-independent approaches is termed “constitutive receptor activation.” In contrast, antagonists can competitively bind to the receptor at the same site as agonists, but do not activate the intracellular response initiated by the active form of the receptor, and therefore inhibit the intracellular responses by agonists.

Certain 2-aminoquinazoline derivatives have been reported to be NPY antagonists which are said to be effective in the treatment of disorders and diseases associated wvith the NPY receptor subtype Y5. See PCT Patent Application 97/20823. Quinazoline derivatives have also been found to be useful by enhancing antitumor activity. See PCT Patent Application 92/07844. And also the quinoline derivatives which have an antagonist activity for MCH receptor are known in these patents, WO0/070244, WO03/105850, WO03/45313, WO03/045920, and WO04/04726.

Recently, our current knowledge of human obesity has advanced dramatically. Previously, obesity was viewed as an oppugnant behavior of inappropriate eating in the setting of appealing foods. Studies of animal models of obesity, biochemical alterations in both humans and animals, and the complex interactions of psychosocial and cultural factors that create receptiveness to human obesity indicate that this disease in humans is multifaceted and deeply entrenched in biologic systems. Thus, it is almost certain that obesity has multiple causes and that there are different types of obesity. Not only does MCHR1 antagonist have potent and durable anti-obesity effects in rodents, it has surprising antidepressant and anxiolytic properties as well (Borowsky et al., Nature Medicine, 8, 825-830, 2002). MCHR1 antagonists have been reported to show antidepressant and anxiolytic activities in rodent models such as social interaction, forced swimming test and ultrasonic vocalization. These findings indicate that MCHR1 antagonists could be useful for treatment of obesity patients with multiple causes. Moreover, MCHR1 antagonists could be used to treat subjects not only with obesity, but also those with depression and anxiety. These advantages make it different from NPY receptor antagonists, with which anxiogenic-like activity can be expected, as NPY itself has anxiolytic-like effect.

Obesity is also regarded as a chronic disease and the possibly of long-term treatment is a concept that is receiving more attention. In this context, it is noteworthy that the depletion of MCH leads to hypophagia as whell as leanness (Shimada et al., Nature, 396, 670-674, 1998). By contrast, NPY (Erickson et al., Nature, 3 S, 415-418, 1996), as well as the Y1 (Pedrazzini et al., Nature Medicine, 4, 722-726, 1998) and Y5 receptors (Marsh et al., Nature Medicine, 4, 718-721, 1998), disrupted mice maintained a stable body weight or rather became obese. Considering the above reports, MCHR1 antagonists can be more attractive than Y1 or Y5 receptor antagonists in terms of long-term treatment of obese patients.

An increasing number of children and adolescents are overweight. Although not all overweight children will necessarily become overweight adults, the growing occurrence of obesity in childhood is likely to be reflected in increasing obesity in adult years. The high prevalence of obesity in our adult population and the likelihood that the nation of the future will be even more obese demands a re-examination of the health implications of this disease. See, Health Implications of Obesity. NIH Consens. Statement Online 1985 Feb. 11-13; 5(9):1-7.

“Clinical obesity” is a measurement of the excess body fat relative to lean body mass and is defined as a body weight more than 20% above the ideal body weight. Recent estimates suggest that 1 in 2 adults in the United States is clinically obese, an increase of more than 25% over the past decades. Flegal M. D. et al., 22 Int. J. Obes. Relat. Metab. Disor. 39 (1998). Both overweight conditions and clinical obesity are a major health concerns worldwide, in particular because clinical obesity is often accompanied by numerous complications, i.e., hypertension and Type II diabetes, which in turn can cause coronary artery disease, stroke, late-stage complications of diabetes and premature death. (See, e.g., Nishina P. M. et al., 43 Metab. 554 (1994)).

Although the etiologic mechanisms underlying obesity require further clarification, the net effect of such mechanisms leads to an imbalance betwveen energy intake and expenditure. Both genetic and environmental factors are likely to be involved in the pathogenesis of obesity. These include excess caloric intake, decreased physical activity, and metabolic and endocrine abnonnalities.

Treatment of overweight conditions and clinical obesity via pharmaceutical agents are not only of importance with respect to the conditions themselves, but also weith respect to the possibility of preventing other diseases that are associated with, e.g., clinical obesity, as well as enhancement of the positive feeling of “self” that often accompanies those who are overweight or clinically obese and who encounter a significant reduction in body weight. Given the foregoing discussion, it is apparent that compounds which help in the treatment of such disorders would be useful and would provide an advance in both research and clinical medicine. The present invention is directed to these, as well as other, important ends.

### SUMMARY OF THE INVENTION

The present invention is drawvn to compounds, which bind to and modulate the activity of a GPCR referred to herein as MCH, and uses thereof. The term MCH, as used herein, includes the human sequences found in GeneBank accession number NM_{—}005297, naturally-occurring allelic variants, mammalian orthologs, biologically active fragments and recombinant mutants thereof.

One aspect of the present invention relates to certain substituted heterocyclic compounds represented by Formula (I): 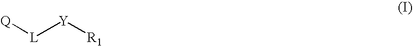
wherein Q is:

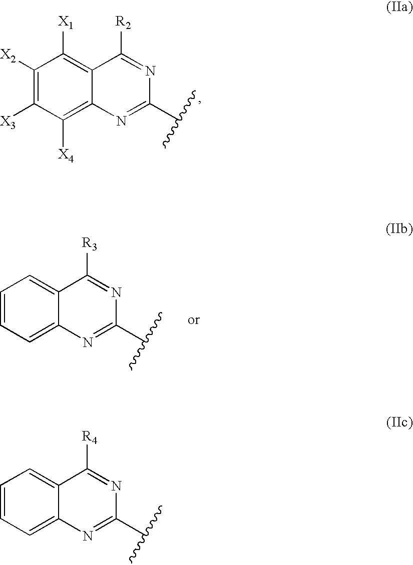

R₁ is selected from the group consisting of:
(i) C₁₋₈ alkyl, and C₁₋₈ alkyl substituted by substituent(s) independently selected from the group consisting of:
   oxo,
   halogen,
   C₁₋₅ alkoxy carbonyl,
   C₁₋₅ alkoxy,
   C₁₋₅ alkoxy substituted by carbocyclic aryl,
   mono-C₁₋₅ allkylamino,
   mono-C₁₋₅ alkylamino substituted by carbocyclic aryl,
   di-C₁₋₅ alkylamino,
   di-C₁₋₅ alkylarnino substituted by carbocyclic aryl,
   C₁₋₅ alkylthio,
   C₃₋₆ cycloalkyl,
   C₃₋₆ cycloalkyl substituted by C₁₋₅ alkyl,
   C₃₋₆ cycloalkenyl,
   carbocyclyl,
   carbocyclic aryl,
   carbocyclic aryl substituted by substituent(s) independently selected from the group consisting of:
      hydroxy,
      halogen,
      nitro,
      amino,
      C₁₋₅ alkylcarbonylamino,
      C₃₋₆ cycloalkylcarbonylamino,
      carbocyclic aryl,
      C₁₋₅ alkyl,
      C₁₋₅ alkyl substituted by halogen,
      C₁₋₅ alkylsulfonyl,
      C₂₋₆alkenyl,
      C₁₋₅ alkoxy, and
      C₁₋₅ alkoxy substituted by halogen,
   mono-carbocyclic arylamino,
   mono-carbocyclic arylamino substituted by substituent(s) independently selected from the group consisting of:
      halogen,
      C₁₋₅ alkyl,
      C₁₋₅ alkyl substituted by halogen,
      C₁₋₅ alkoxy, and
      C₁₋₅ alkoxy substituted by halogen,
   di-carbocyclic arylamino,
   di-carbocyclic arylamino substituted by substituent(s) independently selected from the group consisting of:
      halogen,
      C₁₋₅ alkyl,
      C₁₋₅ alkyl substituted by halogen,
      C₁₋₅ alkoxy, and
      C₁₋₅ alkoxy substituted by halogen,
   carbocyclic aryloxy,
   carbocyclic aryloxy substituted by substituieit(s) independently selected from the group consisting of:
      halogen,
      C₁₋₅ alkyl,
      C₁₋₅ alkyl substituted by halogen,
      C₁₋₅ alkoxy,
      C₁₋₅ sustited by halogen, and
      carbocyclic aryl,
   hydroxy,
   heterocyclyl, and
   heterocyclyl substituted by halogen,
(ii) C₂₋₅ alkenyl, and C₂₋₅ alkenyl substituted by substituent(s) independently selected from the group consisting of:
   oxo, and
   carbocyclic aryl,
(iii) C₂₋₅ alkynyl,
(iv) C₃₋₁₂ cycloalkyl, and C₃₋₁₂ cycloalkyl substituted by carbocyclic aryl,
(v) carbocyclyl, and carbocyclyl substituted by substituent(s) independently selected from the group consisting of:
   hydroxy, and
   carbocyclic aryl,
(vi) carbocyclic aryl, and carbocyclic aryl substituted by substituent(s) independently selected from the group consisting of:
   halogen,
   cyano,
   nitro,
   amino,
   C₁₋₁₀ alkyl,
   C₁₋₁₀ alkyl substituted by substituent(s) independently selected from the group consisting of:
      halogen,
      oxo, and
      carbocyclic aryl,
   carboxy,
   C₁₋₅ alkoxy carbonyl,
      C₁₋₇ alkoxy,
      C₁₋₇ alkoxy substituted by substituent(s) independently selected from the group consisting of:
      halogen, and
      carbocyclic aryl,
   C₃₋₆ cycloalkoxy,
   carbocyclic aryloxy,
   carbocyclic aryloxy substituted by substituent(s) independently selected from the group consisting of:
      halogen,
      nitro,
      C₁₋₅ alkyl,
      C₁₋₅ alkyl substituted by halogen,
      C₁₋₅ alkoxy, and
      C₁₋₅ alkoxy substituted by halogen,
   heterocyclyloxy,
   heterocyclyloxy substituted by substituent(s) independently selected from the group consisting of:
      halogen,
      nitro,
      C₁₋₅ alkyl,
      C₁₋₅ alkyl substituted by halogen,
      C₁₋₅ alkoxy, and
      C₁₋₅ alkoxy substituted by halogen,
   mono-C₁₋₅ alkylamino,
   di-C₁₋₅ alkylamino,
   C₁₋₅ alkylcarbonylamino,
   C₃₋₆ cycloalkylcarbonylamino,
   C₁₋₅ alkoxy carbonylamino,
   carbocyclic aryl azo,
   carbocyclic aryl azo substituted by substituent(s) independently selected from the group consisting of:
      mono-C₁₋₅ alkylamino, and
      di-C₁₋₅ alkylamino,
   C₁₋₅ alkylthio,
   C₁₋₅ alkylthio substituted by halogen,
   carbocyclic arylthio,
   carbocyclic arylthio substituted by nitro,
   amino sulfonyl,
   heterocyclyl sulfonyl,
   C₃₋₆ cycloalkyl,
   C₃₋₆ cycloalkyl substituted by C₁₋₅ alkyl,
   carbocyclic aryl,
   carbocyclic aryl substituted by C₁₋₅ alkoxy,
   hydroxy,
   heterocyclyl, and
   heterocyclyl substituted by C₁₋₅ alkyl,
(vii) heterocyclyl, and heterocyclyl substituted by substitiient(s) independently selected from the group consisting of:
   halogen,
   C₁₋₅ alkyl,
   C₁₋₅ alkyl substituted by halogen,
   C₁₋₅ alkoxy,
   C₁₋₅ alkoxy substituted by halogen,
   C₁₋₅ alkoxy carbonyl,
   C₁₋₅ alkoxy caibon)l substituted by carbocyclic aryl,
   carbocyclic aryloxy,
   carbocyclic aryloxy substituted by substituent(s) independently selected from the group consisting of:
      halogen,
      nitro,
      cyano,
      hydroxy,
      C₁₋₅ alkyl,
      C₁₋₅ alkyl substituted by halogen,
      mono-C₁₋₅ alkylamino,
      di-C₁₋₅ alkylamino,
      C₁₋₅ alkylcarbonylamino,
      C₃₋₆ cycloalkylcarbobylamino,
      C₁₋₅ alkoxy,
      C₁₋₅ alkoxy substituted by halogen,
      C₃₋₆ cycloalkyl,
      C₂₋₅ alkenyl,
      C₂₋₅ alkynyl,
      carboxy,
      C₁₋₅ alkoxycarbonyl,
      mono-C₁₋₅ alkylaminocarbonyl,
      di-C₁₋₅ alkylaminocarbonyl,
      mono-C₃₋₆ cycloalkylaminocarbonyl,
      di-C₃₋₆ cycloalkylaminocarbonyl,
      mono-C₁₋₅ alkylaminocarbonylanmino,
      di-C₁₋₅ alkylaminocarbonylamino,
      mono-C₃₋₆ cycloalkylaminocarbonylamino,
      di-C₃₋₆ cycloalkylaminocarbonylamino,
      C₁₋₅ alkylthio,
      C₁₋₅ alkylthio substituted by halogen,
      C₁₋₅ alkylsulfinyl,
      C₁₋₅ alkylsulfinyl substituted by halogen,
      C₁₋₅ alkylsulfonyl, and
      C₁₋₅ alkylsulfonyl substituted by halogen,
   heterocyclyloxy,
   heterocyclyloxy substituted by substituent(s) independently selected from the group consisting of:
      halogen,
      nitro,
      C₁₋₅ alkyl,
      C₁₋₅ alkyl substituted by halogen,
      C₁₋₅ alkoxy, and
      C₁₋₅ alkoxy substituted by halogen,
   carbocyclic aryl, and
   heterocyclyl;
R₂ is C₁₋₅ alkyl or —N(R₂ₐ)(R_{2b}); wherein R₂ₐ and R_{2b} are independently hydrogen or C₁₋₅ alkyl,
R₃ is C₁₋₅ alkyl;
R₄ is —NHNH₂, —NHNBoc, —N(R₁ₐ)(R_{1b}), morpholino, 4-acetyl-piperazyl, or 4-phenyl-piperazyl; wherein R₁ₐ is hydrogen or C₁₋₅ alkyl; R_{1b} is C₁₋₅ alkyl, C₁₋₅ alkyl substituted by substittent(s) independently selected fronm the group consisting of:
   hydroxy,
   C₁₋₅ alkoxy,
   amino,
   —NHBoc,
   C₃₋₆ cycloalkyl,
   *arbocyclic aryl,
   carbocyclic aryl substituted by substitient(s) independently selected from the group consisting of:
      halogen,
      C₁₋₅ alkyl,
      C₁₋₅ alkoxy, and
      —SO₂NH₂, and
   heterocyclyl,
C₃-₆ cycloalkyl, carbocyclic aryl, carbocyclic aryl substituted by substituent(s) independently selected from the group consisting of:
   halogen,
   C₁₋₅ alkyl,
   C₁₋₅ alkoxy, and
   a group of Formula (III): 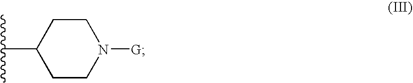
wherein Boc is carbamic acid tert-butyl ester and G is C₁₋₅ alkyl or C₁₋₅ alkeyl substituted by substituent(s) independently selected from the group consisting of:
   carbocyclic aryl,
   halogenated carbocyclic aryl, and
   carbocyclic aryl substituted by C₁₋₅ alkoxy;
L is selected from the group consisting of Formulae (IV) to (XIX): 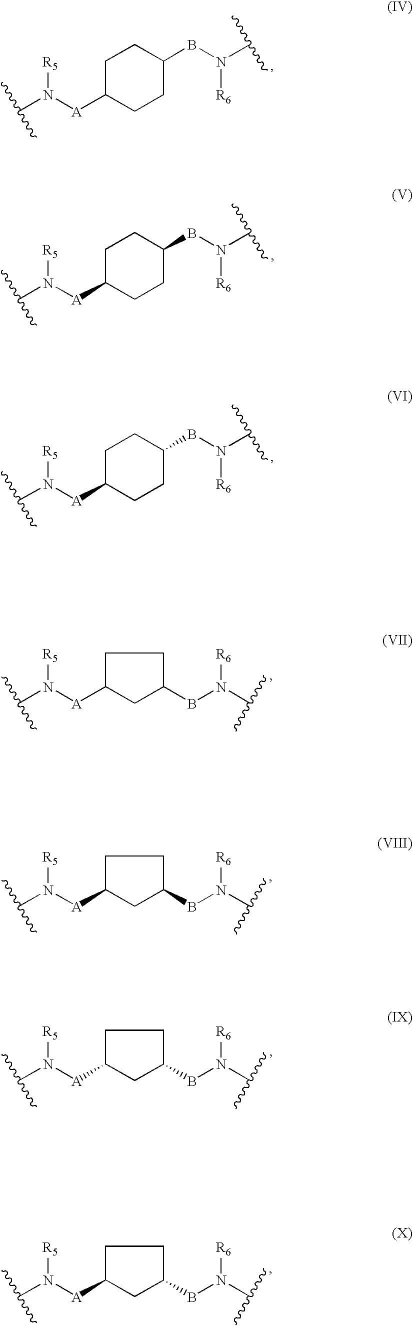 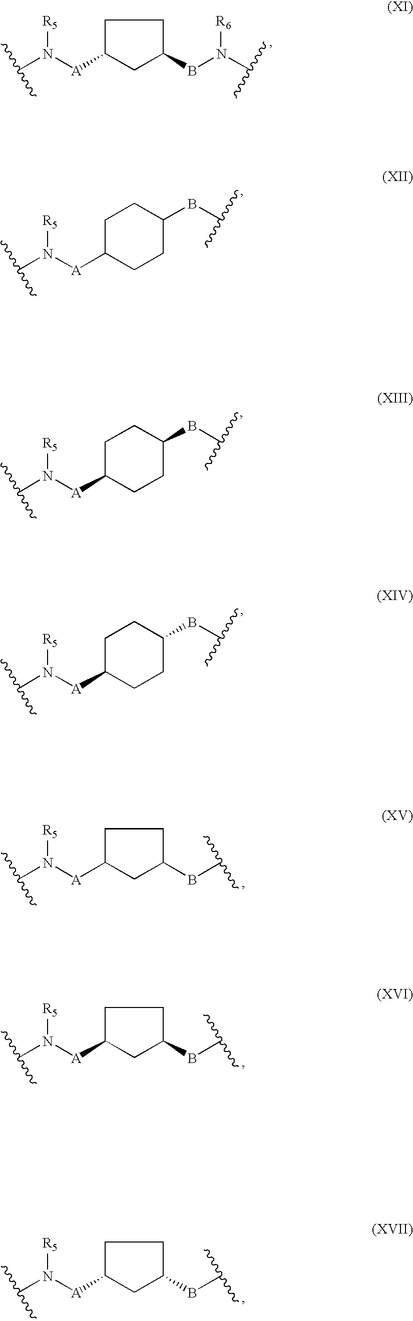 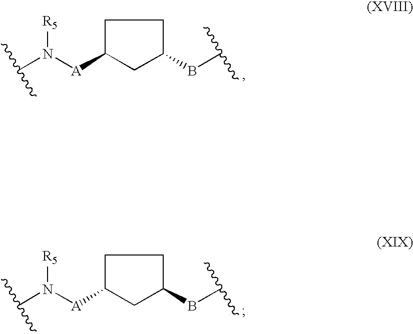
wherein R₅ and R₆ are independently hydrogen or C₁₋₅ alkyl; and A and B are independently a single bond, —CH₂—, or —(CH₂)₂—;
X₁, X₂, X₃ and X₄ are independently selected from the group consisting of hydrogen, halogen, C₁₋₄ alkyl, C₁₋₄ alkyl substituted by halogen, C₁₋₄ alkylthio, C₁₋₄ alkylsulfinyl, C₁₄ alkylsulfonyl, C₁₋₄ alkoxy, C₁₋₄ alkoxy substituted by halogen, nitro, amino, mono-C₁₋₄ alkylamino, di-C₁₋₄ alkylamino, piperidyl, morpholinyl, mono-C-₁₋₄ alkylaminosulfonyyl di-C₁₋₄ alkylaminosulfonyl and hydroxy; provided that at least one substituent selected from the group consisting of X₁, X₂, X₃ and X₄ is not hydrogen; and
Y is selected from the group consisting of:
(i) —C(O)NR₇—, —C(S)NR₇—, or —C(O)O— when L is selected from the group consisting of Formulae (IV) to (XIX); wherein R₇ is hydrogen or C₁₋₅ alkyl;
(ii) —S(O)₂—, —C(O)—, a single bond or —CH₂— when L is selected from the group consisting of Formulae (IV) to (.I), and Q is Formula (IIa) or (IIb);
(iii) —S(O)₂—, —C(O)—, a single bond or —CH₂— when L is selected from the group consisting of Formulae (VII) to (XI), and Q is Formula (IIc); and
(iv) —OC(O)— when L is selected from the group consisting of Formulae (XII) to (XIX);
   wherein carbocyclic aryl is phenyl, naphthyl, or biphenyl;
   carbocyclyl is indanyl, bicyclo[2.2.1]heptyl, bicyclo[2.2.1]heptenyl, adamantly, 9H-fluorenyl, menthyl, 1,2,3,4-tetrahydro-naphthalen-1-yl, or 1H-indolyl;
   heterocyclyl is 2,3-dihydro-benzo[1,4]dioxinyl, 3,4-dihydro-2H-benzo[b][1,4]dioxepinyl, 4,5,6,7-tetrahydro-benzo[b]thienyl, 4H-benzo[1,3]dioxinyl, benzo[1,3]dioxolyl, benzo[2,1,3]thiadiazolyl, benzothiazolyl, furyl, isoxazolyl, morpholinyl, oxazolyl, piperidyl, pyrazolyl, pyridyl, tetrahydrofuryl, thienyl, dibenzofuranyl, 1H-benzoimidazolyl, or thiazolyl; and
   halogen is fluoro, chloro, bromo, or iodo;
or a pharmaceutically acceptable salt, hydrate or solvate thereof.

One aspect of the present invention pertains to pharmaceutical compositions comprising at least one compound, as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, appetite and eatino disorders. cardiovascular disease. hypertension, dyslipidemia myocardial infarction, binge eating disorders including bulimia, anorexia, mental disorders inc;luding manic depression, schizophrenia, delirium, dementia, stress, cognitive disorders, attention deficit disorder, substance abuse disorders and dyskiiiesias including Parkinison's disease, epilepsy, and addiction comprising administering to an individual suffering from said condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a phan-naceutical composition thereof, for use in a method of treatment of the human or animal body by therapy.

One aspect of the present invention pertains to compounds of the present invention, as described herein, or a pharmaceutical composition thereof, for use in a method of prophylaxis or treatment of an eating disorder, obesity or an obesity related disorder of the human or animal body by therapy.

One aspect of the present invention pertains to compounds of the present invention. as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder, obesity or obesity related disorders.

One aspect of the present invention pertains to compounds of the present invention, as described herein, for the manufacture of a medicament for use in the prophylaxis or treatment of anxiety, depression, schizophrenia, addiction or epilepsy.

One aspect of the present hinvention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces weight gain of the individual. In some embodiments, the modulation of the MCH receptor is for prophylaxis or treatment of anxiety, depression, schizophrenia, addiction, or epilepsy.

In some embodiments, the individual is a mammal.

In sone embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound as described herein, and a pharmaceul:ic311 acceptable carrier.

This application claims priority to U.S. Provisional Patent Application, Ser. No. 60/458,424, filed March 31, 2003; and is incorporated herein by reference in its entirety.

### DETAILED DESCRIPTION OF THE INVENTION

One aspect of the present invention relates to certain substituted heterocyclic compounds represented by Formula (I): 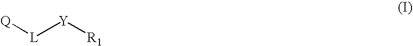
or a pharmaceutically acceptable salt, hydrate or solvate thereof, wherein Q, L, Y, and R₁ are as described herein, supra and infra.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In some embodiments of the present invention, Q is Formulae (IIa), (IIb), or (IIc);

R₁ is selected from the group consisting of:
(i) C₁₋₈ alkyl, and C₁₋₈ alkyl substituted by substituent(s) independently selected from the group consisting of:
   halogen,
   C₁₋₅ alkoxy carbonyl,
   C₁₋₅ alkoxy,
   C₁₋₅ alkoxy substituted by carbocyclic aryl,
   mono-C₁₋₅ alkylamino,
   di-C₁₋₅ alkylamino,
   C₃-₆ cycloalkyl,
   C₃₋₆ cycloalkenyl,
   carbocyclyl,
   carbocyclic aryl,
   carbocyclic aryl substituted by substitulent(s) independently selected from the group consisting of:
      hydroxy,
      halogen,
      nitro,
      C₁₋₅ alkylcarbonylamino,
      C₃₋₆ cycloalkylcarbonylaminio,
      C₁₋₅ alkyl,
      C₁₋₅ alkyl substituted by halogen,
      C₁₋₅ alkylsulfonyl,
      C₂₋₆ alkenyl,
      C₁₋₅ alkoxy,
      C1-5 alkoxy substituted by halogen, and
      carbocyclic aryl,
   heterocyclyl, and
   heterocyclyl substituted by halogen,
(ii) C₂₋₅ alkenyl, and
C₂₋₅ alkenyl substituted by carbocyclic aryl,
(iii) C₂₋₅ alkyynyl,
(iv) C₃₋₁₂ cycloalkyl, and
C₃₋₁₂ cycloalkyl substituted by carbocyclic aryl,
(v) carbocyclyl, and carbocyclyl by substituent(s) independently selected from the group consisting of:
   hydroxy, and
   carbocyclic aryl
(vi) carbocyclic aryl, and carbocyclic aryl substituted by substituent(s) independently selected from the group consisting of:
   halogen,
   cyano,
   nitro,
   C₁₋₁₀ alkyl,
   C₁₋₁₀ alkyl substituted by substituent(s) independently selected from the group consisting of:
      halogen,
      oxo, and
      carbocyclic aryl,
   carboxy,
   C₁₋₅ alkoxy carbonyl,
   C₁₋₇ alkoxy,
   C₁₋₇ alkoxy substituted by substituent(s) independently selected from the group consisting of:
      halogen, and
      carbocyclic aryl,
   carbocyclic aryloxy,
   carbocyclic aryloxy substituted by nitro,
   mono-C₁₋₅ alkylamino,
   di-C₁₋₅ alkylamino,
   C₁₋₅ alkoxy carbonylamino,
   carbocyclic aryl azo,
   carbocyclic aryl azo substituted by substituent(s) independently selected from the group consisting of:
      mono-C₁₋₅ alkylamino, and
      di-C₁₋₅ alkylamino,
   C₁₋₅ alkylthlio,
   C₁₋₅ alkylthio substituted by halogen,
   carbocyclic arylthio,
   carbocyclic arylthio substituted by nitro,
   amino sulfonyl,
   heterocyclyl sulfonyl,
   C₃₋₆ cycloalkyl,
   C₃₋₆ cycloalkyl substituted by C₁₋₅ alkyl,
   carbocyclic aryl,
   heterocyclyl, and
   heterocyclyl substituted by C₁₋₅ alkyl,
(vii) lieterocyclyl, and heterocyclyl substituted by substituent(s) independently selected from the group consisting of:
   halogen,
   C₁₋₅ alkyl,
   C₁₋₅ alkyl substituted by hialogen,
   C₁₋₅ alkoxy,
   C₁₋₅ alkoxy carbonyl,
   C₁₋₅ alkoxy carbonyl substituted by carbocyclic aryl,
   carbocyclic aryloxy,
   carbocyclic aryl, and
   heterocyclyl;
R₂ is —N(R₂ₐ)(R_{2b}), wherein R₂ₐ is hydrogen or C₁₋₅ alkyl; R_{2b} is C₁₋₅ alkyl;
R₃ is C₁₋₅ alkyl;
R₄ is —N(R₄ₐ)(R_{4b}) wherein R₄ₐ is hydrogen or C₁₋₅ alklyl; R_{4b} is C₁₋₅ alkyl;
L is selected from Formula (V), (VIII), (IX ) (XIII), (XVI), or (XVII);
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is selected from the group consisting of:
(i) —C(O)NR₇—, —C(S)NR₇—, or —C(O)O— when L is selected from the group consisting of Formula (V), (VIII), (IX), (XIII), (XVI), or (XVII); wherein R₇ is hydrogen or C₁₋₅ alkyl;
(ii) —S(O)₂—, —C(O)—, a single bond or —CH₂— when L is selected from the group consisting of Formula (VIII) or (IX); and
(iii) —OC(O)— when L is selected from the group consisting of Formula (XIII), (XVI), or (XVII);
   wherein carbocyclic aryl is phenyl or naphthyl;
   carbocyclyl is indanyl, bicyclo[2.2.1]heptyl, bicyclo[2.2.1]heptenyl, adamantly, 9H-flutorenyl, menthyl, 1,2,3,4-tetrahydro-naphthalen-1-yl, or 1H-indolyl,
   heterocyclyl is 2,3-dihydro-benzo[1,4]dioxinyl, 3,4-dihydro-2H-benzo[b][1,4]dioxepinyl, 4,5,6,7-tetrahydro-benzo[b]thienyl, 4H-benzo[1,3]dioxiiiyl, benzo[1,3]dioxolyl, benzo[2,1,3]thiadiazolyl, benzotiliazolyl, furyl, isoxazolyl. morpliolinyl, oxazolyl, piperidyl, pyrazolyl, pyridyl, tetrahydrofuryl, thienyl, dibenzofuranyl, 1H-benzoimidazolyl, or thiazolyl; and
   halogen is fluoro, chloro, bromo, or iodo;
or a pharmaceutically acceptable salt, hydrate or solvate thereof.

In some embodiments of the present invention, Q is Formula (IIc) and can be represented by the following formula: 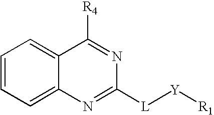
or a pharmaceutically acceptable salt, hydrate or solvate thereof, wherein R₄, L, Y, and R₁ are as described herein, supra and infra.

In some embodiments of the present invention, R₁ is selected from the group consisting of:
(i) C₁₋₅ alkyl, and C₁₋₅ alkyl substituted by substituent(s) independently selected from the group consisting of:
   C₁₋₅ alkoxy carbonyl,
   carbocyclic aryl,
   carbocyclic aryl substituted by substituent(s) independently selected from the group consisting of:
      halogen,
      C₁₋₅ alkyl,
      C₂₋₅ alkenyl, and
      C₁₋₅ alkoxy,
   C₁₋₅ alkyltlilo, and
   heterocyclyl,
(ii) C₃₋₆ cycloalkyl, and C₃₋₆ cycloalkyl substituted by carbocyclic aryl,
(iii) carbocyclyl,
(iv) carbocyclic aryl, and carbocyclic aryl substituted by substitient(s) independently selected from the group consisting of:
   halogen,
   cyano,
   nitro,
   C₁₋₅ alkyl,
   C₁₋₅ alkyl substituted by substituent(s) independently selected from the group consisting of:
      halogen,
      oxo, and
      carbocyclic aryl,
   C₁₋₅ alkoxy carbonyl,
   C₁₋₇ alkoxy,
   C₁₋₇ alkoxy substituted by substituent(s) independently selected from the group consisting of:
      halogen, and
      carbocyclic aryl,
   cycloalkoxy,
   carbocyclic aryloxy,
   mono-C₁₋₅ alkylamino,
   di-C₁₋₅ alkylainino,
   C₁₋₅ alkylthio,
   C₁₋₅ alkylthio substituted by halogen,
   carbocyclic aryl,
   heterocyclyl, and
   heterocyclyl substituted by C₁₋₅ alkyl,
(v) heterocyclyl, and heterocyclyl substituted by substituent(s) independently selected from the group consisting of:
   halogen,
   C₁₋₅ alkyl,
   C₁₋₅ alkyl substituted by halogen,
   C₁₋₅ allkoxy carbonyl
   C₁₋₅ alkoxy carbonyl substituted by carbocyclic aryl, and
   carbocyclic aryl;
L is Formula (V); and
Y is —C(O)NR₇—; wherein R₇ is hydrogen or C₁₋₅ alkyl;
   wherein carbocyclic aryl is phenyl or naphthyl;
   carbocyclyl is indanyl, adamantly, or 9H-fluorenyl;
   heterocyclyl is 2,3-dihydro-benzo[1,4]dioxinyl, 3,4-dihydro-2H-benzo[b][1,4]dioxepinyl, 4H-benzo[1,3]dioxinyl, benzo[1,3]dioxolyl, benzothiazolyl, furyl, isoxazolyl, piperidyl, pyridyl, or thienyl; and
   halogen is fluoro, chloro, broino, or iodo;
or a pharmaceutically acceptable salt, hydrate or solvate thereof

In some embodiments of the present invention, R₁ₐ is hydrogen or methyl; R_{4b} is methyl; R₅ and R₆ are hydrogen; A is a single bond and B is a single bond or —CH₂—; and R₇ is hydrogen; or a pharmaceutically acceptable salt, hydrate or solvate thereof.

In some embodiments of the present invention, R₁ is selected from the group consisting of:
(i) C₁₋₅ alkyl, and C₁₋₅ alkyl substituted by substituent(s) independently selected from the group consisting of:
   C₁₋₅ alkoxy carbonyl,
   carbocyclic aryl,
   carbocyclic aryl substituted by substituent(s) independently selected from the group consisting of:
      halogen,
      C₁₋₅ alkyl,
      C₁₋₅ alkenyl, and
      C₁₋₅ alkoxy,
   C₁₋₅ alkythio, and
   heterocyclyl,
(ii) C₃₋₆ cycloalkyl, and C₃₋₆ cycloalkyl substituted by carbocyclic aryl,
(iii) carbocyclyl,
(iv) carbocyclic aryl, and carbocyclic aryl substituted by substituent(s) independently selected from the group consisting of:
   halogen,
   cyano,
   nitro,
   C₁₋₅ alkyl,
   C₁₋₅ alkyl substituted by halogen
   C₁₋₅ alkoxy carbonyl,
   C₁₋₅ alkoxy,
   C₁₋₅ alkoxy substituted by halogen,
   cycloalkoxy,
   carbocyclic aryloxy,
   C₁₋₅ alkylthio, and
   carbocyclic aryl,
(v) heterocyclyl, and heterocyclyl substituted by substituent(s) independently selected from the group consisting of:
   halogen,
   C₁₋₅ alkyl,
   C₁₋₅ alkyl substituted by halogen, and
   carbocyclic aryl;
   wherein carbocyclic aryl is phenyl or naphthyl;
   carbocyclyl is 9H-fluorenyl;
   heterocyclyl is 2,3-dihydro-benzo[1,4]dioxinyl, 3,4-dihydro-2H-benzo[b][1,4]dioxepinyl, 4H-benzo[1,3]dioxinyl, benzo[1,3]dioxolyl, furyl, isoxazolyl, or thienyl; and
   halogen is fluoro, chloro, bromo, or iodo;
or a pharmaceutically acceptable salt, hydrate or solvate thereof

In some embodiments of the present invention, R₁ is selected from the group consisting of:
(i) C₁₋₅ alkyl, and C₁₋₅ alkyl substituted by substituent(s) independently selected from the group consisting of:
   C₁₋₅ alkoxy carbonyl,
   carbocyclic aryl,
   carbocyclic aryl substituted by substituent(s) independently selected from the group consisting of:
      halogen,
      C₁₋₅ alkyl, and
      C₂₋₅ alkenyl,
   C₁₋₅ alkylthio,
(ii) C₃₋₆ cycloalkyl, and C₃-6 cycloalkyl substituted by carbocyclic aryl,
(iii) carbocyclic aryl, and carbocyclic aryl substituted by substituent(s) independently selected fiom the group consisting of:
   halogen,
   cyano,
   nitro,
   C₁₋₅ alkyl,
   C₁₋₅ alkyl substitited by halogen,
   C₁₋₅ alkoxy carbonyl,
   C₁₋₅ alkoxy,
   cycloalkoxy,
   carbocyclic aryloxy,
   C₁₋₅ alkylthio, and
   carbocyclic aryl,
(iv) heterocyclyl, and heterocyclyl substituted by substituent(s) independently selected from the group consisting of:
   C₁₋₅ alkyl,
   C₁₋₅ alkyl substituted by halogen, and
   carbocyclic aryl;
   wherein carbocyclic aryl is phenyl or naphthyl;
   heterocyclyl is 2,3-dihydro-benzo[1,4]dioxinyl, 3,4-dihydio-2H-benzo[b][1,4]dioxepinyl, benzo[1,3]dioxolyl, furyl, or isoxazolyl; and
   halogen is fluoro, chloro, bromo, or iodo;
or a pharmaceutically acceptable salt, hydrate or solvate thereof

In some embodiments, compounds of the present invention are of Formula (I) wherein the compound is selected from the group consisting of:
N-benzyl-N′-(cis-4-{[4-(dimethylamino)quinazolin-2-yl]amino}cyclohexyl)urea;
N-(2-bromophenyl)-N′-(cis-4-{[4-(dimethylamino)quinazolin-2-yl]amino}cyclohexyl)urea;
N-biphenyl-2-yl-N′-(cis-4-{[4-(dimethylamino)quinazolin-2-yl]amyino}cyclohexyl)urea;
N-(4-bromophenyl)-N′-(cis-4-{[4-(dimethylamino)quinazolin-2-yl]amino}cyclohexyl)urea;
N-butyl-N′-(cis-4-{[4-(dimethylamino)quinazolin-2-yl]amino}cyclohexyl)urea;
N-cyclohexyl-N′-(cis-4-{[4-(dimethylamino)quinazolin-2-yl]amino}cyclohexyl)urea;
N-(2-chlorophetyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,6-dimethylphenyl)-urea;
N-(2,4-difluorophenyl)-N′-(cis-4-{[4-(dimethylamino)quinazolin-2-yl]amino}cyclohexyl)-urea;
N-(2,4-dichlorophenyl)-N′-(cis-4-{[4-(dimethylamino)quinazolin-2-yl]amino)-cyclohlexyl)-urea;
N-(cis-4-{[4-(dimethylamino)quinazolin-2-yl]amino}cyclohexyl)-N′-(2,3-dimethylphenyl)-urea;
ethyl 3-({[(cis-4-{[4-(dimethylamino)quinazolin-2-yl]amino}cyclohexyl)amino]carbonyl}-amino)benzoate;
ethyl 4-({[(cis-4-{[4-(dimethylamino)quinazolin-2-yl]amino}cyclohexyl)amino]carbonyl}-amino)benzoate;
N-(cis-4-{[4-(dimethylamino)quinazolin-2-yl]amino}cyclohexyl)-N′-(4-ethylphenyl)urea;
N-(cis-4-{[4-(dimethylamino)quinazolin-2-yl]amino}cyclohexyl)-N′-(2-ethyl-6-methylphenyl)urea;
ethyl N-{[(cis-4-{[4-(dimethylamino)quinazolin-2-yl]amino}cyclohexyl)amino]carbonyl}-leucinate;
N-(cis-4-{[4-(dimethylamino)quinazolin-2-yl]amino}cyclohexyl)-N′-(4-fluorophenyl)urea;
N-(cis-4-{[4-(dimethylamino)quinazolin-2-yl]amino) cyclohexyl)-N′-[1-(3-isopropenylphenyl)-1-methylethyl]urea;
methyl N-{[(cis-4-{[4-(dimethylamino)quinazolin-2 -yl]amino}cyclohexyl)amino]carbonyl}-methioninate;
N-(cis-4-{[4-(dimethylamino)quinazolin-2-yl]amino}cyclohexyl)-N′-(4-methoxyphenyl)-urea;
N-(cis-4-{[4-(dimethylamino)quinazolin-2-yl]aminolcyclohexyl)-N′-(2-methoxyphenyl)-urea;
N-(cis-4-}[4-(dimethylamino)quinazolin-2-yl]amino}cyclohexyl)-N′-(3-nmethoxyphenyl)-urea;
N-(cis-4-{[4-(dimethylamino)quinazolin-2-yl]amino}cyclohexyl)-N′-[4-(methylthio)-phenyl]urea;
N-(cis-4-{[4-(dimethylamino)quinazolin-2-yl]amino}cyclohexyl)-N′-(4-methoxybenzyl)-urea;
N-(cis-4-{[4-(dimethylamino)quinazolin-2-yl]amino}cyclohexyl)-N′-1 -naphthylurea;
N-(cis-4-{[4-(dimethylamino)quinazolin-2-yl]amino}cyclohexyl)-N′-[(2S)-2-phenylcyclopropyl]urea;
N-(cis-4-{[4-(dimethylamino)quinazolin-2-yl]amino}cyclohexyl)-N′-phenylurea;
N-(cis-4-{[4-(dimethylamino)quinazolin-2-yl]amino}cyclohexyl)-N′-(4-phenoxyphenyl)-urea;
N-(cis-4-{[4-(dimethylamino)quinazolin-2-yl]amino}cyclohexyl)-N′-pentylurea;
N-(cis-4-{[4-(dimethylamino)quinazolin-2-yl]amino}cyclohexyl)-N′-[2-(trifluoromethyl)-phenyl]urea;
N-(cis-4-{[4-(dimethylamino)quinazolin-2-yl]amino}cyclohexyl)-N′-(4-methylphenyl)urea;
N-(cis-4-{[4-(dimethylamino)quinazolin-2-yl]amino}cyclohexyl)-N′-mesitylurea;
N-(cis-4-{[4-(dimethylamino)quinazolin-2-yl]amino}cyclolhexyl)-N′-(3-methylphenyl)urea;
N-(cis-4-{[4-(dimethylamino)quinazolin-2-yl]amino}cyclohexyl)-N′-(2-methylphenyl)urea;
N-(cis-4-{[4-(dimethylamino)quinazolin-2-yl]amino}cyclohexyl)-N′-[1-(1-naphthyl)ethyl]-urea;
methyl N-{[(cis-4-{[4-(dimethylamino)quinazoiin-2-yl]amino}cyclohexyl)amino]carbonyl)-phenylalaninate;
N-(cis-4-{[4-(dimethy lamino)quinazlin-2-yl]am iiio cyclohexyl)-N′-(2,4,6-trichlorophenyl)urea;
N-(cis-4-}[4-(dimethylamino)quiniazolin-2-yl]amino}cyclohexyl)-N′-(1-phenyl-ethyl)urea;
1-[4-(4-dimethylamino-quinazolin-2-ylamino)-cyclohexyl]-3-(1-phenyl-ethyl)-urea;
1-[4-(4-dimethylamino-quinazolin-2-ylamino)-cyclohexyl]-3-(1-naphthalen-1-yl-ethyl)-urea;
N-(cis-4-{[4-(dimethylamino)quinazolin-2-yl]amino}cyclohexyl)-N′-[2-(methylthio)-phenyl]urea;
N-(cis-4-{[4-(dimethylamino)quinazolin-2-yl]amino}cyclohexyl)-N′-(2,3,5,6-tetrachlorophenyl)urea;
N-(cis-4-{[4-(dimethylamino)quinazolin-2-yl]amino}cyclohexyl)-N′-(2,3-dimethyl-6-nitrophenyl)urea;
N-(cis-4-{[4-(dimethylamino)quinazolin-2-yl]amino}cyclohexyl)-N′-(2,4,6-tribromophenyl)urea;
N-(2,4-dibromo-6-fluorophenyl)-N′-(cis-4-([4-(dimethylamino)quinazolin-2-yl]amino}-cyclohexyl)urea;
N-(2,4-dibromophenyl)-N′-(cis-4-{[4-(dimethylamino)quinazolin-2-yl]amino}cyclohexyl)-urea;
N-(2,4-dichlorobenzyl)-N′-(cis-4-{[4-(dimethylamino)quinazolin-yl]amino}cyclohexyl)-urea;
N-(2,4-dimethoxyphenyl)-N′-(cis-4-{[4-(dimethylamino)quinazolin-2-yl]amino}-cyclohexyl)urea;
N-(2,5-dimethoxyphenyl)-N′-(cis-4-{[4-(dimethylamino)quinazolin-2-yl]amino}-cyclohexyl)urea;
N-(7,6-diethylphenyl)-N′-(cis-4-{[4-(dimethylamino)qu iiazolin-2-yl]amino}cyclohexyl)-urea;
N-(2-chloro-5-nitrophenyl)-N′-(cis-4-{[4-(dimethylamino)quinazolin-2-yl]amino)-cyclohexyl)urea;
N-[2-chloro-6-(trifluoromethyl)phenyl]-N′-(cis-4-{[4-(dimethylamino)quinazolin-2-yl]amino}cyclohexyl)urea;
N-(2-chloro-6-methylphenyl)-N′-(cis-4-([4-(dimethylamino)quinazolin-2-yl)amino}-cyclohexyl)urea;
N-(2-chlorobenzyl)-N′-(cis-4-{[4-(dimethylamino)quinazolin-2-yl]amino}cyclohexyl)urea;
N-(cis-4-{(4-(dimethylamino)quinazolin-2-yl]amino}cyclohexyl)-N′-(2-ethoxyphenyl)urea;
N-(cis-4-{[4-(dimethylamino)quinazolin-2-yl]amino}cyclohexyl)-N′-(2-ethyl-6-isopropylphenyl)urea;
N-(cis-4-{[4-(dimethylamino)quinazolin-2-yl]amino}cyclohexyl)-N′-(2-ethylphenyl)urea;
N-(cis-4-{[4-(dimethylamino)quinazolin-2-yl]amino}cyclohexyl)-N′-(2-fluorobenzyl)urea;
N-(cis-4-{[4-(dimethylamino)quinazolin-2-yl]amino}cyclohexyl)-N′-(2-iodophenyl)urea;
N-(cis-4-{[4-(dimethylamino)quinazolin-2-yl]amino}cyclohexyl)-N′-(2-isopropyl-6-methylphenyl)urea;
N-(cis-4-{[4-(dimethylamino)quinazolin-2-yl]amino}cyclohexyl)-N′-(2-isopropylphenyl)-urea;
N-(cis-4-{[4-(dimethylamino)quinazolin-2-yl]amino}cyclohexyl)-N′-(2-methoxy-4-nitrophenyl)urea;
N-(cis-4-{[4-(dimethylamino)quinazolin-2-yl]amino}cyclohexyl)-N′-(2-methoxy-5-methylphenyl)urea;
N-(cis-4-{[4-(dimethylamino)quinazolin-2-yl]amino}cyclohexyl)-N′-(2-methyl-3-nitrophenyl)urea;
N-(cis-4-{[4-(dimethylamino)quiiazolin-2-yl]amino}cyclohexyl)-N′-(2-methyl-4-nitrophenyl)urea;
N-(cis-4-{[4-(dimethylamino)quinazolin-2-yl]amino}cyclohexyl)-N′-(2-methyl-5-nitrophenyl)urea;
N-(cis-4-{[4-(dimethylamino)quinazolin-2-yl]amino}cyclohexyl)-N′-(2-methylbenzyl)urea;
N-(cis-4-{[4-(dimethylamino)quinazolin-2-yl]amino}cyclolexyl)-N′-(2-nitrophenyl)urea;
N-(cis-4-{[4-(dimethylamino)quinazolin-2-yl]amino}cyclohexyl)-N′-(2-propylphenyl)urea;
N-(cis-4-{[4-(dimethylamino)quinazolin-2-yl]amino}cyclohexyl)-N′-(2-phenoxyphenyl)-urea;
N-(2-tert-butyl-6-methylphenyl)-N′-(cis-4-{[4-(dimethylamino)quinazolin-2-yl]amino}cyclohexyl)urea;
N-(2-tert-butylphenyl)-N′-(cis-4-{[4-(dimethylamino)quinazolin-2-yl]amino}cyclohexyl)-urea;
N-(cis-4-{[4-(dimethylamino)quinazolin-2-yl]amino) cyclohexyl)-N′-[3-(methylthio)-phenyl]urea;
N-1,3-benzodioxol-5-yl-N′-(cis-4-{[4-(dimethylamino)quinazolin-2-yl]amino}cyclohexyl)-urea;
N-(cis-4-{[4-(dimethylamino)quinazolin-2-yl]amino}cyclohexyl)-N′-(3,4,5-trimethoxyphenyl)urea;
N-(3,4-dichlorobenzyl)-N′-(cis-4-{[4-(dimethylamino)quinazolin-2-yl]amino}cyclohexyl)-urea;
N-(3,4-difluorophenyl)-N′-(cis-4-([4-(dimethylamino)quinazolin-2-yl]amino}cyclohexyl)-urea;
N-(3,4-dimethoxyphenyl)-N′-(cis-4-{[4-(dimethylamino)quinazolin-2-yl]amino}-cyclohexyl)urea;
N-(3,5-difluorophienyl)-N′-(cis-4-{[4-(dimethylamino)quinazolin-2-yl]amino}cyclohexyl)-urea;
N-(3,5-dimethoxyphenyl)-N′-(cis-4-{[4-(dimethylamino)quinazolin-2-yl]amino}-cyclohexyl)urea;
N-(cis-4-{[4-(dimethylamino)quinazolin-2-yl)amino}cyclohexyl)-N′-(3,5-dimethylphenyl)-urea;
methyl 3-({[(cis-4-{[4-(dimethylamino)quinazolin-2-yl]amino) cyclohexyl)amino]carbonyl}-amino)benzoate;
N-(3-chloro-2-methylphenyl)-N′-(cis-4-{[4-(dimethylamino)quinazolin-2-yl]amino}-cyclohexyl)urea;
N-(3-chloro-4-fluorophenyl)-N′-(cis-4-{[4-(dimethylamino)quinazolin-2-yl]amino}-cyclohexyl)urea;
N-(3-chloro-4-methoxyphenyl)-N′-(cis-4-{[4-(dimethylamino)quiniizolin-2-yl]amino}-cyclolexyl)urea;
N-(cis-4-{[4-(dimethylamino)quinazolin-2-yl]amino}cyclohexyl)-N′-(3-ethylphenyl)urea;
N-(cis-4-{[4-(dimethylamino)quinazolin-2-yl]amnio )cyclolhexyl)-N′-(3-fluorobenzyl)urea;
N-[4-bromo-2-(trifluoromethyl)phenyl]-N′-(cis-4-{[4-(dimethylamino)quinazolin-2-yl]amino}cyclohexyl)urea;
N-(4-bronio-2,6-difluorophenyl)-N′-(cis-4-{[4-(dimethylamino)quinazolin-2-yl]amino}-cyclohexyl)urea;
N-(4-bromobenzyl)-N′-(cis-4-{[4-(dimethylamino)quinazolin-2-yl]amino}cyclohexyl)urea;
N-[4-chloro-2-(trifluoromethyl)phenyl]-N′-(cis-4-{[4-(dimethylamino)quinazolin-2-yl]amino}cyclohexyl)urea;
N-(4-chloro-2-methylphenyl)-N′-(cis-4-{[4-(dimetlhylamino)q uI iazolin-2-yl]amino}-cyclohexyl)urea;
N-(4-cyanophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(4-ethoxyphenyl)urea;
N-(cis-4-{[4-(dimethylamino)quinazolin-2-yl]amino}cyclohexyl)-N′-(4-fluoro-2-nitrophenyl)urea;
N-(cis-4-}[4-(dimethylamino)quinazolin-2-yl]amino}cyclolexyl)-N′-(4-fluorobenzyl)urea;
N-(cis-4-{[4-(dimethylamino)quinazolin-2-yl]amino}cyclohexyl)-N′-(4-iodophenyl)urea;
N-(cis-4-{[4-(dimethylamino)quinazolin-2-yl]amino}cyclohexyl)-N′-(4-methoxy-2-methylphenyl)urea;
N-(cis-4-{[4-(dimethylamino)quinazolin-2-yl]amino}cyclohexyl)-N′-(4-methylbenzyl)urea;
N-(5-chloro-2,4-dimethoxyphenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-(5-fluoro-2-methylphenyl)urea;
N-(cis-4-{[4-(dimethylamino)quinazolin-2-yl]amino}cyclohexyl)-N′-9H-fluoren-9-ylurea;
N-(cis-4-{[4-(dimethylamino)quinazolin-2-yl]amino}cyclohexyl)-N′-(2-phenylethyl)urea;
N-cyclopentyl-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(diphenylmethyl)urea;
N-[1-(4-bromophenyl)ethyl]-N′-(cis-4-{[4-(dimethylamino)quinazolin-2-yl]amino}-cyclohexyl)urea;
N-(4-bromo-2,6-dimethylphenyl)-N′-(cis-4-{[4-(dimethylamino)quinazolin-2-yl]amino}-cyclohexyl)urea;
N-(4-bromo-2-methylphetnyl)-N′-(cis-4-{[4-(dimethylamino)quinazolin-2-yl]amino}-cyclohexyl)urea;
ethyl N-{[(cis-4-{[4-(dimetylamino)quinazolin-2-yl]amino}cyclohexyl)amino]carbonyl}-phenylalaninate;
N-(cis-4-{[4-(dimethylamino)quinazolin-2-yl]amino}cyclolexyl)-N′-[2-(2-thienyl)ethyl]-urea;
N-(2,3-dihydro-1,4-benzodioxin-6-yl)-N′-(cis-4-{[4-(dimethylamino)quinazolin-2-yl]amino}cyclohexyl)urea;
N-(2,6-dibromo-4-isopropylphenyl)-N′-(cis-4-{[4-(dimethylamino)quinazolin-2-ylamino]-cyclohexyl}urea;
N-[3-(cyclopentyloxy)-4-methoxyphenyl]-N′-(cis-4-{[4-(dimethylamino)quinazolin-2-yl]amino}cyclohexyl)urea;
N-(3,4-dihydro-2H-1,5-benzodioxepin-7-yl)-N′-(cis-4-{[4-(dimethylamino)quinazolin-2-yl]amino}cyclohexyl)urea;
N-(4-butyl-2-methylphenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-[5-methyl-2-(trifluoromethyl)-3-furyl]urea;
N-(cis-4-{[4-(dimethylamino)quinazolin-2-yl]amino) cyclohexyl)-N′-(6-fluoro-4H-1,3-benzodioxin-8-yl)urea;
N-(cis-4-{[4-(dimethylamino)quinazolin-2-yl]amino}cyclohexyl)-N′-(3,5-dimethylisoxazol-4-yl)urea;
N-(cis-4-{[4-(dimethylamino)quinazolin-2-yl]amino}cycloiexyl)-N′-(3-methyl-5-phenylisoxazol-4-yl)urea;
N-(cis-4-((4-(dimethylamino)quinazolin-2-yl]amino}cyclohexyl)-N′-(5-methyl-3-phenylisoxazol-4-yl)urea;
N-(2-bromophenyl)-N′-[(cis-4-{[4-(dimethylamino)quinazolin-2-yl]amino}cyclohexyl)-methyl]urea;
N-biphenyl-2-yl-N′-[(cis-4-{[4-(dimethylamino)quinazolin-2-yl]amino}cyclohexyl)methyl]-urea;
N-butyl-N′-[(cis-4-{[4-(dimethylamino)quinazolin-2-yl]amino}cyclohexyl)methyl]urea;
N-(3-chlorophenyl)-N′-[(cis-4-{[4-(dimethylamino)quinazolin-2-yl]amino}cyclohexyl)-methyl]urea;
N-cyclohlexyl-N′-[(cis-4-{[4-(dimethylamino)quinazolin-2-yl]aminio}cyclohexyl)methyl]-urea;
N-(3-cyanophenyl)-N′-[(cis-4-{[4-(dimethylamino)quinazolin-2-yl]amino}cyclohexyl)-methyl]urea;
N-(2-chlorophenyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl]-N′-(2,6-dimethylphenyl)urea;
N-(3,4-dichlorophenyl)-N′-[(cis-4-{[4-(dimethylamino)quinazolin-2-yl]amino}cyclohexyl)-methyl]urea;
N-(2,4-difluorophenyl)-N′-[(cis-4-{[4-(dimethylamino)quinazolin-2-yl]amino}cyclohexyl)-mnethyl]urea;
N-(2,4-dichlorophenyl)-N′-[(cis-4-{[4-(dimethylamino)quinazolin-2-yl]amino}cyclohexyl)-methyl]urea;
N-(3,5-dichlorophenyl)-N′-[(cis-4-{[4-(dimethylamino)quinazolin-2-yl]amino}cyclohexyl)-methyl]urea;
N-(2,3-dichlorophenyl)-N′-[(cis-4-{[4-(dimethylamino)quinazolin-2-yl]amino}cyclohexyl)-methyl]urea;
N-(2,6-difluorophenyl)-N′-[(cis-4-{[4-(dimethylamino)quinazolin-2-yl]amino}-cyclohexyl)-methyl]urea;
N-[(cis-4-{[4-(dimethylamino)quinazolin-2-yl]amino}cyclohexyl)methyl]-N′-(2,3-dimethylphenyl)urea;
N-[(cis-4-{[4-(dimethylamino)quinazolin-2-yl]amino}cyclohexyl)methyl]-N′-(4-ethylphenyl)urea;
N-[(cis-4-{[4-(dimethylanino)quinazolin-2-yl]amino}cyclohexyl)methyl]-N′-(2-ethyl-6-methylphenyl)urea;
ethyl N-({[(cis-4-{[4-(dimethylamino)quinazolin-2-yl]amino}cyclohexyl)methyl]amino)-carbonyl)leucinate;
N-[(cis-4-{[4-(dimethylamino)quinazolin-2-yl]amino}cyclohexyl)methyl]-N′-(4-fluorophenyl)urea;
N-[(cis-4-{[4-(dimethylamino)quinazolin-2-yl]amino}cyclohexyl)methyl]-N′-(3-fluorophenyl)urea;
N-[(cis-4-{[4-(dimethylamino)quinazolin-2-yl]amino}cyclohexyl)methyl]-N′-(2-fluorophenyl)urea;
N-[(cis-4-{[4-(dimethylamino)quinazolin-2-yl]amino₁cyclohexyl)methyl]-N′-[1-(3-isopropenylphenyl)-1-methylethyl]urea;
methyl N-({[(cis-4-{[4-(dimethylamino)quiniazolin-2-yl]amino}cyclohexyl)methyl]amino}-carbonyl)methioninate;
N-[(cis-4-{[4-(dimethylamino)quinazolin-2-yl]amino}cyclohexyl)methyl]-N′-(4-methoxyphenyl)urea;
N-[(cis-4-{[4-(dimethylamino)quinazolin-2-yl]amino}cyclohexyl)methyl]-N′-(4-methyl-2-nitrophenyl)urea;
N-[(cis-4-{[4-(dimethylamino)quinazolin-2-yl]amino}cyclohexyl)methyl]-N′-(2-methoxyphenyl)urea;
N-[(cis-4-{[4-(dimethylamino)quinazolin-2-yl]amino}cyclohexyl)methyl]-N′-(3-methoxyphenyl)urea;
N-[(cis-4-{[4-(dimethylamino)quinazolin-2-yl]amino}cyclohexyl)methyl]-N′-[4-(methylthio)phenyl]urea;
N-[(cis-4-{[4-(dimethylamino)quinazolin-2-yl]amino}cyclohexyl)methyl]-N′-1-naphthylurea;
N-[(cis-4-{[4-(dimethylamino)quinazolin-2-yl]amino}cyclohexyl)methyl]-N′-phenylurea;
N-[(cis-4-{[4-(dimethylamino)quinazolin-2-yl]amino}cyclohexyl)methyl]-N′pentylurea;
N-[(cis-4-{[4-(dimethylamino)quinazolin-2-yl]amino}cyclohexyl)methyl]-N′-[2-(trifluoromethyl)phenyl]urea;
N-[(cis-4-}[4-(dimethylamino)quinazolin-2-yl]amino}cyclohexyl)methyl]-N′-(4-methylphenyl)urea;
N-[(cis-4-{[4-(dimethylamino)quinazolin-2-yl]amino}cyclohexyl)methyl]-N′-mesitylurea;
N-[(cis-4-{[4-(dimethylamino)quinazolin-2-yl]amino}cyclohexyl)methyl]-N′-(3-methylphenyl)urea;
N-[(cis-4-{[4-(dimethylamino)quinazolin-2-yl]amino}cyclohexyl)methyl]-N′-(2-methylphenyl)urea;
N-[(cis-4-{[4-(dimethylamino)quinazolin-2-yl]amino}cyclohexyl)methyl]-N′-(2,4,6-trichlorophenyl)urea;
N-[(cis-4-{[4-(dimethylamino)quinazolin-2-yl]amino}cyclohexyl)methyl]-N′-(1-phenylethyl)urea;
1-[4-(4-dimethylamino-quinazolin-2-ylamino)-cyclohexylmethyl]-3-(1-phenyl-ethyl)-urea;
1-[4-(4-dimethylamino-quinazolin-2-ylamino)-cyclohexylnethyl]-3-(1-naphthalen-1-yl-ethyl)-urea;
N-(2,6-diisopropylphenyl)-N′-[(cis-4-{[4-(dimethylamino)quinazolin-2-yl]amino}-cyclohexyl)methyl)urea;
N-[2-(difluoromethoxy)phenyl]-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-2-(methylthio)phenyl]urea;
N-[(cis-4-{[4-(dimethylamino)quinazolin-2-yl]amino}cyclohexyl)methyl)]-N′-(2,3,5,6-tetrachlorophenyl)urea;
N-[(cis-4-{[4-(dimethylamino)quiniazolin-2-yl]amino}cyclohexyl)methyl]-N′-(2,3-dimethyl-6-nitrophenyl)urea;
N-[(cis-4-{[4-(dimethylamino)quinazolin-2-yl]amino}cyclohexyl)methyl]-N′-(2,4,6-tribromophenyl)urea;
N-(2,4-dibromo-6-fluorophenyl)-N′-[(cis-4-{[4-(dimethylamino)quinazolin-2-yl]amino}-cyclohexyl)methyl]urea;
N-(2,4-dichlorobenzyl)-N′-[(cis-4-{[4-(dimethylamino)quinazolin-2-yl]amino}cyclohexyl)-methyl]urea;
N-(2,5-dimethoxyphenyl)-N′-[(cis-4-{[4-(dimethylamino)quinazolin-2-yl]amino}-cyclohexyl)methyl]urea;
N-(2.6-dibromo-4-fluorophenyl)-N′-[(cis-4-{[4-(dimethylamino)quinazolin-2-yl]amino}-cyclohexyl)methyl]urea;
N-(2,6-dichlorophenyl)-N′-[(cis-4-{[4-(dimethylamino)quinazolin-2-yl]amino}cyclohexyl)-methyl]urea;
N-(2,6-diethylphenyl)-N′-[(cis-4-{[4-(dimethylamino)quinazolin-2-yl]amino}cyclohexyl)-methyl]urea;
N-(2-chloro-5-methylphenyl)-N′-[(cis-4-{[4-(dimethylamino)quinazolin-2-yl]amino}-cyclohexyl)methyl]urea;
N-[2-chloro-6-(trifluoromnethyl)phenyl]-N′-[(cis-4-{[4-(dimethylamino)quinazolin-2-yl]-amino}cyclohexyl)methyl]urea;
N-(2-chloro-6-methylphenyl)-N′-[(cis-4-{[4-(dimethylamino)quinazolin-2-yl]amino}-cyclohexyl)methyl]urea;
N-(2-chlorobenzyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl]-N′-(2-ethy)-6-isopropylphenyl)urea;
N-[(cis-4-{[4-(dimethylamino)quinazolin-2-yl]amino}cycliohexyl)metlyl]-N′-(2-ethylphenyl)urea;
N-[(cis-4-{[4-(dimethylamino)quinazolin-2-yl]amino}cyclohexyl)methyl]-N′-(2-fluoro-5-nitrophenyl)urea;
N-[(cis-4-{[4-(dimethylamino)quinazolin-2-yl]amino}cyclohexyl)methyl]-N′-(2-fluorobenzyl)urea;
N-[(cis-4-{[4-(dimethylamino)quinazolin-2-yl]amino}cyclohexyl)methyl]-N′-(2-iodophenyl)urea;
N-[(cis-4-{[4-(dimethylamino)quinazolin-2-yl]amino}cyclohexyl)mehhyl]-N′-(2-isopropyl-6-methylphenyl)urea;
N-[(cis-4-{[4-(dimethylamino)quinazolin-2-yl]amino}cyclohexyl)methyl]-N′-(2-isopropylphenyl)urea;
N-[(cis-4-{[4-(dimethylamino)quinazolin-2-yl]amino}cyclohexyl)methyl]-N′-(2-methoxy-5-methylphenyl)urea;
N-[(cis-4-{[4-(dimethylamino)quinazolin-2-yl]amino}cyclohexyl)methyl]-N′-(2-methoxy-5-nitrophenyl)urea;
N-[(cis-4-{[4-(dimethylamino)quinazolin-2-yl]amino}cyclohexyl)methyl]-N′-(2-methyl-3-nitrophenyl)urea;
N-[(cis-4-{[4-(dimethylamino)quinazolin-2-yl]amino}cyclohexyl)methyl]-N′-(2-methyl-4-nitrophenyl)urea;
N-[(cis-4-{[4-(dimethylamino)quinazolin-2-yl]amino}cyclohexyl)methyl]-N′-(2-methyl-5-nitrophenyl)urea;
N-[(cis-4-{[4-(dimethylamino)quinazolin-2-yl]amino}cyclohexyl)methyl]-N′-(2-methyl-6-nitrophenyl)urea;
N-[(cis-4-{[4-(dimethylamino)quinazolin-2-yl]amino}cyclohexyl)methyl]-N′-(2-methylbenzyl)urea;
N-[(cis-4-{[4-(dimethylamino)quinazolin-2-yl]amino}cyclohexyl)methyl]-N′-(2-nitrophenyl)urea:
N-[(cis-4-{[4-(dimethylamino)quinazolin-2-yl]amino}cyclohexyl)methyl]-N′-(2-propylphenyl)urea;
N-[(cis-4-{[4-(dimethylamino)quinazolin-2-yl]amino}cyclolexyl)methyl]-N′-(2-phenoxyphenyl)urea;
N-(2-tert-butyl-6-methylphenyl)-N′-[(cis-4-{[4-(dimethylamino)quinazolin-2-yl]amino}-cyclohexyl)methyl]urea;
N-(2-tert-butylphenyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl]-N′-[3-(methylthio)phlenyl]urea;
N-(3,4-difluorophenyl)-N′-[(cis-4-{[4-(dimethylamino)quinazolin-2-yl]amino}cyclohexyl)-methyl]urea;
N-(3,5-difluorophenyl)-N′-[(cis-4-{[4-(dimethylamino)quinazolin-2-yl]amino}cyclohexyl)-methyl]urea;
N-(3,5-dimetlioxyphenyl)-N′-[(cis-4-{[4-(dimethylamino)quinazolin-2-yl]amino}-cyclohexyl)methyl]urea;
N-(3-chloro-2-methylphenyl)-N′-[(cis-4-{[4-(dimethylamino)quinazolin-2-yl]amino}-cyclohexyl)methyl]urea;
N-(3-chloro-4-fluorophenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl)-N′-(3-ethylphenyl]urea;
N-[(cis-4-{[4-(dimethylamino)quinazolin-2-yl]amino}cyclohexyl)methyl)-N′-(3-fluoro-5-(trifluoromethyl)phenyl]urea;
N-[(cis-4-{[4-(dimethylamino)quinazolin-2-yl]amino}cyclohexyl)methyl]-N′-(3-fluorobenzyl)urea;
N-[(cis-4-{[4-(dimethylamino)quinazolin-2-yl]amino}cyclohexyl)methyl]-N′-(4,5-dimethyl-2)-nitrophenyl)urea;
N-[4-bromo-2-(trifluoromethyl)phenyl]-N′-[(cis-4-{[4-(dimethylamino)quinazolin-2-yl]amino}cyclohexyl)methyl]urea;
N-(4-bromno-2,6-difluorophenyl)-N′-[(cis-4-{[4-(dimethylamino)quinazolin-2-yl]amino}-cyclohexyl)methyl]urea;
N-[4-chloro-2-(trifluorometlyl)phenyl]-N′-[(cis-4-{[4-(dimethylamino)quinazolin-2-yl]amino}cyclohexyl)methyl]urea;
N-(4-chloro-2-methlylphenyl)-N′-[(cis-4-{[4-(dimethylamino)quinazolin-2-yl]amino}-cyclohexyl)methyl]urea;
N-(4-cyanophenyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl)-N′-(4-fluoro-2-nitrophenyl]urea;
N-[(cis-4-{[4-(dimethylamino)quinazolin-2-yl]amino}cyclohexyl)methyl)-N′-(4-fluorobenzyl]urea;
N-[(cis-4-{[4-(dimethylamino)quinazolin-2-yl]amino}cyclohexyl)methyl]-N′-(4-iodophenyl)urea;
N-[(cis-4-{[4-(dimethylamino)quinazolin-2-yl]amino}cyclohexyl)methyl]-N′-(4-methoxy-2-methylphenyl)urea;
N-[(cis-4-{[4-(dimethylamino)quinazolin-2-yl]amino}cyclohexyl)methyl]-N′-(4-methyl-3-nitrophenyl)urea;
N-(5-chloro-2-methylphenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-(5-fluoro-2-methylphenyl)urea;
N-cyclopentyl-N′-[(cis-4-{[4-(dimethylamino)quinazolin-2-yl]amino}cyclohexyl)methyl]-urea;
N-[(cis-4-{[4-(dimethylamino)quinazolin-2-yl]amino}cyclohexyl)methyl)-N′-(diphenlylmethyl)urea;
N-(4-bromo-2,6-dimethylphenyl)-N′-[(cis-4-{[4-(dimethylamino)quinazolin-2-yl]amino}-cyclohexyl)methyl]urea;
N-(4-bromo-2-methylphenyl)-N′-[(cis-4-{[4-(dimethylamino)quiniazolin-2-yl]amino}-cyclohexyl)methyl]urea;
N-(2,6-dibromo-4-isopropylphenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-3-thienylurea;
N-[(cis-4-{[4-(dimethylamino)quinazolin-2-yl]amino}cyclohexyl)methyl]-N′-[5-methyl-2-(trifluoromethyl)-3-furyl]urea;
N-[(cis-4-{[4-(dimethylamino)quinazolin-2-yl]amino}cyclohexyl)methyl]-N′-(6-fluoro-4H-1,3-benzodioxin-8-yl)urea;
N-[(cis-4-{[4-(dimethylamino)quinazolin-2-yl]amino}cyclohexyl)methyl]-N′-(3,5-dimethylisoxazol-4-yl)urea;
N-[(cis-4-{[4-(dimethylamino)quinazolin-2-yl]amino}cyclohexyl)methyl]-N′-(3-methyl-5-phenylisoxazol-4-yl)urea;
N-[(cis-4-{[4-(dimethylamino)quinazolin-2-yl]amino}cyclohexyl)methyl]-N′-(5-methyl-3-phenylisoxazol-4-yl)urea; and
N-(cis-4-{[4-(dimethylamino)quinazolin-2-yl]amino}cyclohexyl)-N′-[3-(trifluoromethoxy)-phenyl]urea;

or a pharmaceutically acceptable salt, hydrate or solvate thereof.

In some embodiments, compounds of the present invention are of Formula (I) wherein the compound is selected from the group consisting of:
N-(2-bromophenyl)-N′-(cis-4-{[4-(dimethylamino)quinazolin-2-yl]amino}cyclohexyl)urea;
N-biphenyl-2-yl-N′-(cis-4-{[4-(dimethylamino)quinazolin-2-yl]amino}cyclohexyl)urea;
N-butyl-N′-(cis-4-{[4-(dimethylamino)quinazolin-2-yl]amino}cyclohexyl)urea;
N-(2-chlorophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,6-dimetlylphenyl)-urea;
N-(2,4-difluorophenyl)-N′-(cis-4-{[4-(dimethylamino)quinazolin-2-yl]amino}cyclohexyl)-urea;
N-(cis-4-{[4-(dimethylamino)quinazolin-2-yl]amino}cyclohexyl)-N′-(2,3-dimethylphenyl)-urea;
ethyl 3-{[(cis-4-{[4-(dimethylamino)quinazolin-2-yl]amino}cyclohexyl)amino]carbonyl}-amino)benzoate;
N-(cis-4-{[4-(dimethylamino)quinazolin-2-yl]amino}cyclohexyl)-N′-(2-ethyl-6-methylphenyl)urea;
ethylN-{[(cis-4-{[4-(dimethylamino)quinazolin-2-yl]-amino}cyclohexyl)amino]-carbonyl}leucinate;
N-(cis-4-{[4-(dimethylamino)quinazolin-2-yl]amino}cyclohexyl)-N′-(4-fluorophenyl)urea;
N-(cis-4-{[4-(dimethylamino)quinazolin-2-yl]amino}cyclohexyl)-N′-[1-(3-isopropenylphenyl)-1-methylethyl]urea;
methyl N-{[(cis-4-{[4-(dimethylamino)quinazolin-2-yl]amino}cyclohexyl)amino]-carbonyl}methioninate;
N-(cis-4-{[4-(dimethylamino)quinazolin-2-yl]amino}cyclohexyl)-N′-[4-(methylthio)phenyl]urea;
N-(cis-4-{[4-(dimethylamino)quinazolin-2-yl]amino}cyclohexyl)-N′-1-naphthylurea;
N-(cis-4-{[4-(dimethylamino)quinazolin-2-yl]amino}cyclohexyl)-N′-[(2S)-2-phenylcyclopropyl]urea;
N-(cis-4-{[4-(dimethylamino)quinazolin-2-yl]amino}cyclohexyl)-N′-(4-phenoxyphenyl)urea;
N-(cis-4-{[4-(dimethylamino)quinazolin-2-yl]amino}cyclohexyl)-N′-pentylurea;
N-(cis-4-{[4-(dimethylamino)quinazolin-2-yl]amino}cyclohexyl)-N′-[2-(trifluoromethyl)-phenyl]urea;
N-(cis-4-{[4-(dimethylamino)quinazolin-2-yl]amino}cyclohexyl)-N′-mesitylurea;
N-(cis-4-{[4-(dimethylamino)quinazolin-2-yl]amino}cyclohexyl)-N′-(2-methylphenyl)urea;
N-(cis-4-{[4-(dimethylamino)quinazolin-2-yl]amino}cyclohe-:yl)-N′-[1-(1-naphthyl)ethyl]-urea;
methyl N-{[(cis-4-{[4-(dimethylamino)quinazolin-2-yl]amino}cyclohexyl)amino]carbonyl}-phenylalaninate;
N-(cis-4-{[4-(dimethylamino)quinazolin-2-yl]amino}cyclohexyl)-N′-(2,4,6-trichlorophenyl)urea;
N-(cis-4-{[4-(dimethylamino)quinazolin-2-yl]amino}cyclohexyl)-N′-(1-phenylethyl)urea;
1-(4-Dimethylamino-quinazolin-2-ylamino)-cyclohexyl]-3-(1-phenyl-ethyl)-urea;
N-(cis-4-{[4-(dimethylamino)quinazolin-2-yl]amino}cyclohexyl)-N′-(2,3,5,6-tetrachlorophenyl)urea;
N-(cis-4-{[4-(dimethylamino)quinazolin-2-yl]amino}cyclohexyl)-N′-(2,4,6-tribromophenyl)urea;
N-(2,4-dibromo-6-fluorophenyl)-N′-(cis-4-{[4-(dimethylamino)quinazolin-2-yl]amino}-cyclohexyl)urea;
N-(2,4-dibromophenyl)-N′-(cis-4-{[4-(dimethylamino)quinazolin-2-yl]amino}cyclohexyl)-urea;
N-(2,4-dichlorobenzyl)-N′-(cis-4-{[4-(dimethylamino)quinazolin-2-yl]amino}cyclohexyl)-urea;
N-(2,4-dimethoxyphenyl)-N′-(cis-4-{[4-(dimethylamino)quinazolin-2-yl]amino}-cyclohexyl)urea;
N-(2,6-diethylphenyl)-N′-(cis-4-{[4-(dimethylamino)quinazolin-2-yl]amino) cyclohexyl)-urea;
N-[2-chloro-6-(trifluoromethyl)phenyl]-N′-(cis-4-{[4-(dimethylamino)quinazolin-2-yl]-amino}cyclohexyl)urea;
N-(2-chloro-6-methylphenyl)-N′-(cis-4-{[4-(dimethylamino)quinazolin-2-yl]amino}-cyclohexyl)urea;
N-(2-chlorobenzyl)-N′-(cis-4-{[4-(dimethylamino)quinazolin-2-yl]amino}cyclohexyl)urea;
N-(cis4-{[4-(dimethylamino)quinazolin-2-yl]amino}cyclohexyl)-N′-(2-ethoxyphenyl)urea:
N-(cis-4-{[4-(dimethylamino)quinazolin)-2-yl]amino}cyclohexyl)-N′-(2-ethyl-6-isopropylphenyl)urea;
N-(cis-4-{[4-(dimethylamino)quinazolin-2-yl]amino}cyclohexyl)-N′-(2-ethylphenyl)urea;
N-(cis-4-{[4-(dimethylamino)quinazolin-2-yl]amino}cyclohexyl)-N′-(2-fluorobenzyl)urea;
N-(cis-4-{[4-(dimethylamino)quinazolin-2-yl]amino}cyclohexyl)-N′-(2-iodophenyl)urea;
N-(cis-4-{[4-(dimethylamino)quinazolin-2-yl]amino}cyclohexyl)-N′-(2-isopropyl-6-mnethylphenyl)urea;
N-(cis-4-{[4-(dimethylamino)quinazolin-2-yl]amino}cyclohexyl)-N′-(2-isopropylphenyl)-urea;
N-(cis-4-{[4-(dimethylamino)quinazolin-2-yl]amino}cyclolexyl)-N′-(2-methyl-3-nitrophenyl)urea;
N-(cis-4-{[4-(dimethylamino)quinazolin-2-yl]amino}cyclohexyl)-N′-(2-methyl-4-nitrophenyl)urea;
N-(cis-4-{[4-(dimethylamino)quinazolin-2-yl]amino}cyclohexyl)-N′-(2-methyl-5-nitrophenyl)urea;
N-(cis-4-{[4-(dimethylamino)quinazolin-2-yl]amino}cyclohexyl)-N′-(2-methylbenzyl)urea;
N-(cis-4-{[4-(dimethylamino)quinazolin-2-yl]amino}cyclohexyl)-N′-(2-nitrophenyl)urea;
N-(cis-4-{[4-(dimethylamino)quinazolin-2-yl]amino}cyclohexyl)-N′-(2-propylphenyl)urea;
N-(2-tert-butyl-6-methylphenyl)-N′-(cis-4-{[4-(dimethylamino)quinazolin-2-yl]amino}-cyclohexyl)urea;
N-(2-tert-butylphenyl)-N′-(cis-4-{[4-(dimethylamino)quinazolin-2-yl]amino}cyclohexyl)-urea;
N-1,3-benzodioxol-5-yl-N′-(cis-4-{[4-(dimethylamino)quinazolin-2-yl]amino}cyclohexyl)-urea;
N-(cis-4-{[4-(dimethylamino)quinazolin-2-yl]amino}cyclohexyl)-N′-(3,4,5-trimethoxyphenyl)urea;
N-(3,4-dimethoxyphenyl)-N′-(cis-4-{[4-(dimethylamino)quinazolin-2-yl]amino}-cyclohexyl)urea;
N-(3-chloro-2-methylphenyl)-N′-(cis-4-{[4-(dimethylamino)quinazolin-2-yl]amino}-cyclohexyl)urea;
N-(3-chloro-4-methoxyphenyl)-N′-(cis-4-{[4-(dimethlylamino)quinazolin-2-yl]amino}-cyclohexyl)urea;
N-[4-bromo-2-(trifluoromethyl)phenyl]-N′-(cis-4-{[4-(dimethylamino)quinazolin-2-yl]-amino}cyclohexyl)urea;
N-(4-bromo-2,6-difluorophenyl)-N′-(cis-4-[4-(dimethylamino)quinzolin-]amino}-cyclohexyl)urea;
N-(4-bromobenzyl)-N′-(cis-4-{[4-(dimethylamino)quinazolin-2-yl]amino}cyclohexyl)urea;
N-[4-chloro-2-(trifluoromethyl)phenyl]-N′-(cis-4-{[4-(dinethylamino)quinazolin-2-yl]-amino}cyclohexyl)urea;
N-(4-chloro-2-methylphenyl)-N′-(cis-4-{[4-(dimethylamino)quinazolin-2-yl]amino}-cyclohexyl)urea;
N-(4-cyanophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(4-fluorobenzyl)urea;
N-(cis-4-{[4-(dimethylamino)quinazolin-2-yl]amino}cyclohexyl)-N′-(4-methoxy-2-methylphenyl)urea;
N-(5-chloro-2,4-dimethoxyphenyl)-N′-(cis-4-{[4-(dimethylamino)quinazolin-2-yl]amino}-cyclohexyl)urea;
N-(cis-4-{[4-(dimethylamino)quinazolin-2-yl]amino) cyclohexyl)-N′-(diphenylmethyl)urea;
N-[1-(4-bromophenyl)ethyl]-N′-(cis-4-{[4-(dimethylamino)quinazolin-2-yl]amino}-cyclohexyl)urea;
N-(4-bromo-2,6-dimethylphenyl)-N′-(cis-74-{[4-(dimethylamino)quinazolin-2-yl]amino}-cyclohexyl)urea;
N-(4-bromo-2-metylphenyl)-N′-(cis-4-{[4-(dimethylamino)quinazolin-2-yl]amino}-cyclohexyl)urea;
ethyl N-{[(cis-4-{[4-(dimethylamino)quinazolin-2-yl]amino}cyclohexyl)amino]carbonyl}-phenylalaninate;
N-(2,3-dihydro-1,4-benizodioxin-6-yl)-N′-(cis4-{[4-(dimethylamino)quinazolin-2-yl]amino}cyclohexyl)urea;
N-(2,6-dibromo-4-isopropylphenyl)-N′-(cis-4-{[4-(dimethylamino)quinazolin-2-yl]amino}cyclohexyl)urea;
N-[3-(cyclopentyloxy)-4-methoxyphenyl]-N′-(cis-4-{[4-(dimethylamino)quinazolin-2-yl]amino}cyclohexyl)urea;
N-(3,4-dihydro-2H-1,5-benzodioxepin-7-yl)-N′-(cis-4-{[4-(dimethylamino)quinazolin-2-yl]amino}cyclohexyl)urea;
N-(4-butyl-2-methylphlenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-[5-methyl-2-(trifluoromethyl)-3-furyl]urea;
N-(cis-4-{[4-(dimethylamino)quinazolin-2-yl]amino) cyclohexyl)-N′-(3-methyl-5-phenylisoxazol-4-yl)urea;
N-(cis-4-{[4-(dimethylamino)quinazolin-2-yl]amino) cyclohexyl)-N′-(5-methyl-3-phenylisoxazol-4-yl)urea;
N-(2-chlorophenyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl]-N′-(2,6-dimethylphenyl)urea;
N-(2,4-difluorophenyl)-N′-[(cis-4-{[4-(dimethylamino)quinazoliln-2-yl]amino}cyclohexyl)-methyl]urea;
N-(3,5-dichlorophenyl)-N′-[(cis-4-{[4-(dimethylamino)quiniazolin-2-yl]amino}cyclohexyl)-methyl]urea;
N-(2,3-dichlorophenyl)-N′-[(cis-4-{[4-(dimethylamino)quinazolin-2-yl]amio}cyclohexyl)-methyl]urea;
N-[(cis-4-{[4-(dimethylamino)quinazolin-2-yl]amino}cyclohexyl)methyl]-N′-(2,3-dimethylphenyl)urea;
N-[(cis-4-{[4-(dimethylamino)quinazolin-2-yl)ainiilo cyclohexyl)methyl]-N′-(2-ethyl-6-metlylphenyl]urea;
ethyl N-({[(cis-4-{[4-(dimethylamino)quinazolin-2-yl]amino) cyclohexyl)methyl]amino}-carbonyl)leucinate;
N-[(cis-4-{[4-(dimethylamino)quinazolin-2-yl]amino}cyclohexyl)methyl]-N′-(4-fluorophenyl)urea;
N-[(cis-4-{[4-(dimethylamino)quinazolin-2-yl]amino}cyclohexyl)methyl]-N′-[4-(methylthio)phenyl]urea;
N-[(cis-4-{[4-(dimethylamino)quinazolin-2-yl]amino}cyclohexyl)methyl]-N′-phenylurea;
N-[(cis-4-{[4-(dimethylamino)quinazolin-2-yl]amino}cyclohexyl)methyl]-N′-[2-(trifluoromethyl)phenyl]urea;
N-[(cis-4-{[4-(dimethylamino)quinazolin-2-yl]amino}cyclohexyl)methyl]-N′-(4-methylphenyl)urea;
N-[(cis-4-{[4-(dimethylamino)quinazolin-2-yl]amino}cyclohexyl)methyl]-N′-mesitylurea;
N-[(cis-4-{[4-(dimethylamino)quinazolin-2-yl]amino}cyclohexyl)methyl]-N′-(2-methylphenyl)urea;
N-[(cis-4-{[4-(dimethylamino)quinazolin-2-yl]amino}cyclohexyl)methyl]-N′-(2,4,6-trichlorophenyl)urea;
N-(2,6-diisopropylphenyl)-N′-[(cis-4-{[4-(dimethylamino)quiniazolin-2-yl]amino}-cyclohexyl)methyl]urea;
N-[(cis-4-{[4-(dimethylamino)quinazolin-2-yl]amino}cyclohexyl)methyl]-N′-(2,3-dimethyl-6-nitrophenyl)urea;
N-[(cis-4-{[4-(dimethylamino)quinazolin-2-yl] amino}cyc liohexyl)methyl]-N′-(2,4,6-tribroinophenyl)urea;
N-(2,4-dibromo-6-fluorophenyl)-N′-[(cis-4-{[4-(dimethylamino)quinazolin-2-yl]amino}-cyclohexyl)methyl]urea;
N-(2,6-dibromo-4-fluorophenyl)-N′-[(cis-4-{[4-(dimethylamino)quinazolin-2-yl]amino}-cyclohexyl)methyl)urea;
N-(2,6-dichlorophenyl)-N′-[(cis-4-{[4-(dimethylamino)quinazolin-2-yl]amino}cyclohexyl)-methyl]urea;
N-(2,6-diethylphenyl)-N′-[(cis-4-{[4-(dimethylamino)quinazolin-2-yl]amino}cyclohexyl)-methyl)urea;
N-[2-chloro-6-(trifluoromethyl)phenyl]-N′-[(cis-4-}[4-(dimethylamino)quinazolin-2-yl]-amino}cyclohexyl)methyl]urea;
N-(2-chloro-6-methylphenyl)-N′-[(cis-4-{[4-(dimethylamino)quinazolin-2-yl]amino}-cyclohexyl)methyl]urea;
N-(2-chlorobenzyl)-N′-[(cis-4-{[4-(dimethylamino)quinazolin-2-yl]amino}cyclohexyl)-methyl]urea;
N-[(cis-4-{[4-(dimethylamino)quinazolin-2-yl]amino}cyclohexyl)methyl]-N′-(2-ethyl-6-isopropylphenyl)urea;
N-[(cis-4-{[4-(dimethylamino)quinazolin-2-yl]amino}cyclohexyl)methyl]-N′-(2-ethylphenyl)urea;
N-[(cis-4-{[4-(dimethylamino)quinazolin-2-yl]amino}cyclohexyl)methyl]-N′-(2-iodophenyl)urea;
N-[(cis-4-{[4-(dimethylamino)quinazolin-2-yl]amino}cyclohexyl)methyl]-N′-(2-isopropyl-6-methylphenyl)urea;
N-[(cis-4-{[4-(dimethylamino)quinazolin-2-yl]amino}cyclohexyl)methyl]-N′-(2-isopropylphenyl)urea;
N-[(cis-4-{[4-(dimethylamino)quinazolin-2-ylamino}cyclohexyl)methyl]-N′-(2-methoxy-5-methylphenyl)urea;
N-[(cis-4-{[4-(dimethylamino)quinazolin-2-yl]amino) cyclohexyl)methyl]-N′-(2-methyl-3-nitrophenyl)urea;
N-[(cis-4-{[4-(dimethylamino)quinazolin-2-yl]amino}cyclohexyl)methyl]-N′-(2-methyl-6-nitrophenyl)urea;
N-[(cis-4-{[4-(dimethylamino)quinazolin-2-yl]amino}cyclohexyl)methyl)-N′-(2-propylphenyl)urea;
N-(2-tert-butyl-6-methylphenyl)-N′-[(cis-4-{[4-(dimethylamino)quinazolin-2-yl]amino}-cyclohexyl)methyl]urea;
N-(2-tert-butylphenyl)-N′-[(cis-4-{[4-(dimethylamino)quinazolin-2-yl]amino}cyclohexyl)-methyl]urea;
N-(3,4-difluorophenyl)-N′-[(cis-4-{[4-(dimethylamino)quinazolin-2-yl]amino}cyclohexyl)-methyl)urea;
N-(3,5-difluorophenyl)-N′-[(cis-4-{[4-(dimethylamino)quinazolin-2-yl]amino}cyclohexyl)-methyl]urea;
N-(3-chloro-2-methylphenyl)-N′-[(cis-4-{[4-(dimethylamino)quinazoln-2-yl]amino}-cyclohexyl)methyl]urea;
N-(3-chloro-4-fluorophenyl)-N′-[(cis-4-{[4-(dimethylamino)quinazolin-2-yl]amino}-cyclohexyl)methyl]urea;
N-(4-bromo-2,6-difluorophenyl)-N′-[(cis-4-{[4-(dimethylamino)quinazolin-2-yl]amino}-cyclohexyl)methyl]urea;
N-[4-chloro-2-(trifluoromethyl)phenyl]-N′-[(cis-4-{[4-(dimethylamino)quinazolin-2-yl]-amino}cyclolhexyl)methyl]urea;
N-(4-cyanophenyl)-N′-[(cis-4-{[4-(dimethylamino)quinazolin-2-yl]amino}cyclohexyl)-methyl]urea;
N-[(cis-4-((4-(dimethylamino)quinazolin-2-yl]amino}cyclohexyl)methyl]-N′-(diphenylmetlhyl)urea;
N-(4-bromo-2,6-dimethylphenyl)-N′-[(cis-4-{ (4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-[5-methyl-2-(trifluoromethyl)-3-furyl]urea; and
N-[(cis-4-{[4-(dimethylamino)quinazolin-2-yl]amino) cyclohexyl)methyl]-N′-(3-methyl-5-phenylisoxazol-4-yl)urea;

or a pharmaceutically acceptable salt, hydrate or solvate thereof.

In some embodiments of the present invenltion, R₁ is selected fiom the group consisting of:
(i) C₁₋₈ alkyl, and C₁₋₈ alkyl substituted by substituent(s) independently selected from the group consisting of:
   mono-C₁₋₅ alkylamino,
   di-C₁₋₅ alkylamino,
   C₃₋₆ cycloalkyl,
   C₃₋₆ cycloalkenyl
   carbocyclic aryl,
   carbocyclic aryl substituted by substituent(s) independently selected from the group consisting of:
      halogen,
      C₁₋₅ alkyl, and
      C₁₋₅ alkoxy,
   heterocyclyl,
(ii) C₂₋₅ alkynyl,
(iii) C₂₋₅ alkenyl, and C₂₋₅ alkenyl substituted by carbocyclic aryl,
(iv) C₃₋₁₂ cycloalkyl,
(V) carbocyclyl,
(vi) carbocyclic aryl, and carbocyclic aryl substituted by substituent(s) independently selected from the group consisting of:
   halogen,
   cyano,
   nitro,
   C₁₋₁₀ alkyl,
   C₁₋₁₀ alkyl substituted by substituent(s) independently selected from the group consisting of:
      halogen, and
      oxo,
   carboxy,
   C₁₋₅ alkoxy carbonyl,
   C₁₋₅ alkoxy,
   C₁₋₅ alkoxy substituted by substituent(s) independently selected from the group consisting of:
      halogen, and
      carbocyclic aryl,
   carbocyclic aryloxy,
   carbocyclic aryloxy substituted by nitro,
   mono-C₁₋₅ alkylamino,
   di-C₁₋₅ alkylamino,
   C₁₋₅ alkoxy carbonylamino,
   carbocyclic aryl azo,
   carbocyclic aryl azo substituted by substituent(s) independently selected from the group consisting of:
      mono-C₁₋₅ alkylamino, and
      di-C₁₋₅ alkylamino,
   C₁₋₅ alkylthio,
   C₁₋₅ alkylthio substituted by halogen,
   carbocyclic arylthio,
   carbocyclic arylthio substituted by nitro,
   amino sulfonyl,
   heterocyclyl sulfonyl,
   C₃₋₆ cycloalkyl,
   C₃₋₆ cycloalkyl substituted by C₁₋₅ alkyl,
   carbocyclic aryl, and
   heterocyclyl,
(vii) heterocyclyl, and heterocyclyl substituted by substituent(s) independently selected from the group consisting of:
   C₁₋₅ alkyl,
   C₁₋₅ alkoxy carbonyl,
   carbocyclic aryloxy,
   carbocyclic aryl, and
   heterocyclyl;
L is Forrnula (V); and
Y is —C(S)NR₇—; wherein R₇ is hydrogen or C₁₋₅ alkyl;
wherein carbocyclic aryl is phenyl or naphthyl;
carbocyclyl is indanyl, bicyclo[2.2.1]heptyl, bicyclo[2.2.1]heptenyl, or adamantly;
heterocyclyl is 2,3-dihydro-benzo[1,4]dioxinyl, 4,5,6,7-tetrahydi-o-benzo[b]thienyl, benzo[ l ,]dioxolyl, benzo[2, 1 ,3]thiadiazolyl, furyl, isoxazolyl, morpliolinyl, oxazolyl. piperidyl, pyrazolyl, pyridyl, tetrahydrofuryl, or thienyl; and
halogen is fluoro, chloro, bromo, or iodo;

or a pharmaceutically acceptable salt, hydrate or solvate thereof.

In some embodiments of the present invention, R₄ₐ is hydrogen or methyl; R_{4b} is methyl; R₅ and R₆ are hydrogen; A is a single bond; B is a single bond or —CH₂—; and R₇ is hydrogen; or a pharmaceutically acceptable salt, hydrate or solvate thereof.

In some embodiments of the present invention, R₁ is selected from the group consisting of:
(i) C₁₋₆ alk-yl, and C₁₋₆ alkyl substituted by substituent(s) independently selected from the group consisting of:
   C₃₋₆ cycloalkyl,
   C₃₋₆ cycloalkenyl,
   carbocyclic aryl,
   carbocyclic aryl substituted by substituent(s) independently selected from the group consisting of:
      halogen,
      C₁₋₅ alkyl, and
   heterocyclyl,
(ii) C₃₋₁₂ cycloalkyl,
(iii) carbocyclyl,
(iv) carbocyclic aryl, and carbocyclic aryl substituted by substituent(s) independently selected from the group consistingc of:
   halogen,
   cyano,
   nitro,
   C₁₋₅ alkyl,
   C₁₋₅ alkyl substituted by halogen,
   C₁₋₅ alkoxy carbonyl,
   C₁₋₅ alkoxy,
   C₁₋₅ alkoxy substituted by halogen,
   mono-C₁₋₅ alkylamino,
   di-C₁₋₅ alkylamino,
   C₁₋₅ alkylthio, and
   carbocyclic aryl,
(v) heterocyclyl, and heterocyclyl substituted by substitlent(s) independently selected from the group consisting of:
   C₁₋₅ alkoxy carbonyl, and
   carbocyclic aryl;
      wherein carbocyclic aryl is phenyl or naphthyl;
      carbocyclyl is indanyl, bicyclo[2.2.1]heptyl, or bicyclo[2.2.1]heptenyl;
      heterocyclyl is 2,3-dihydro-benzo[1,4]dioxinyl, benzo[1,3]dioxolyl, isxoazolyl, tetrahydrofuryl, or thienyl; and
      halogen is fluoro, chloro, bromno, or iodo;
or a pharmaceutically acceptable salt, hydrate or solvate thereof.

In some embodiments of the present invention, R₁ is selected from the group consisting of:
(i) C₁₋₅ alkyl, and C₁₋₅ alkyl substituted by substituent(s) independently selected from the group consisting of:
   carbocyclic aryl,
   carbocyclic aryl substituted by substituent(s) independently selected from the group consisting of:
      halogen, and
      C₁₋₅ alkoxy;
(ii) carbocyclyl,
(iii) carbocyclic atyl, and carbocyclic aryl substituted by substituent(s) independently selected from the group consisting of:
   halogen,
   cyano,
   nitro,
   C₁₋₅ alkyl,
   C₁₋₅ alkyl substituted by halogen,
   C₁₋₅ alkoxy carbonyl,
   C₁₋₅ allkoxy,
   C₁₋₅ alkoxy substituted by halogen,
   mono-C₁₋₅ alkylamino,
   di-C₁₋₅ alkylamrino, and
   carbocyclic aryl,
(iv) heterocyclyl, and heterocyclyl substituted by substituent(s) independently selected from the group consisting of:
   C₁₋₅ alkyl,
   C₁₋₅ alkoxy carbonyl, and
   carbocyclic aryl; wherein carbocyclic aryl is phenyl or naphthyl;
   carbocyclyl is bicyclo[2.2.1]heptyl;
   heterocyclyl is 2,3-dihydro-benzo[1,4]dioxinyl, benzo[1,3]dioxolyl, isoxazolyl, or thienyl; and
   halogen is fluoro, chloro, bromo, or iodo;
or a pharmaceutically acceptable salt, hydrate or solvate thereof.

In some embodiments, compounds of the present invention are of Formula (I) wherein the compound is selected from the group consisting of:
N-(4-bromophenyl)-N′-(cis-4-{[4-(dimethylamino)quinazolin-2-yl]amino}cyclohexyl)-thiourea;
N-(4-cyanophenyl)-N′-(cis-4-{[4-(dimethylamino)quinazolin-2-yl]amino}cyclohexyl)-thiourea;
N-cyclohexyl-N′-(cis-4-{[4-(dimethylamino)quinazolin-2-yl]amino}cyclohexyl)thiourea;
N-cyclopentyl-N′-(cis-4-{[4-(dimethylamino)quinazolin-2-yl]amino}cyclohexyl)thiourea;
N-(4-chlorophenyl)-N′-(cis-4-{[4-(dimethylamino)quinazolin-2-yl]amino}cyclohexyl)-thiourea;
N-(2,4-dichlorophenyl)-N′-(cis-4-{[4-(dimethylamino)quinazolin-2-yl]amino}cyclohexyl)-thiourea;
N-(2,4-dimnethoxyphenyl)-N′-(cis-4-{[4-(dimethylamino)quinazolinn-yl)]amino}-cyclohexyl)thiourea;
N-(cis-4-}[4-(dimethylamino)quinazolin-2-yl]amino}cyclohexyl)-N′-(2,6-dimethylphenyl)-thiourea;
N-(cis-4-{[4-(dimethylamino)quinazolin-2-yl]amino}cyclohexyl)-N′-(2-ethyl-6-isopropylphenyl)thiourea;
N-(cis-4-{[4-(dimethylamino)quinazolin-2-yl]amino}cyclohexyl)-N′-(4-fluorophenyl)-thiourea;
N-(cis-4-{[4-(dimethylamino)quinazolin-2-yl]amino}cyclohexyl)-N′-hexylthiourea;
N-(cis-4-{[4-(dimethylamino)quinazolin-2-yl]amino}cyclohexyl)-N′-isobutylthiourea;
N-(cis-4-{[4-(dimethylamino)quinazolin-2-yl]amino}cyclohexyl)-N′-(4-methoxybiphenyl-3-yl)thiourea;
N-(1,3-benzodioxol-5-ylmethyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-[4-(methylthio)pheny)]-thiourea;
N-(cis-4-{[4-(dimethylamino)quinazolin-2-yl]amino}cyclohexyl)-N′-(4-methoxyphenyl)-thiourea;
N-(cis-4-{[4-(dimethylamino)quinazolin-2-yl]amino}cyclohexyl)-N′-(2-methoxyphenyl)-thiourea;
N-(cis-4-{[4-(dimethylamino)quinazolin-2-yl]amino}cyclohexyl)-N′-1-naphthylthiourea;
N-(cis-4-{[4-(dimethylamino)quinazolin-2-yl]amino) cyclohexyl)-N′-(4-nitrophenyl)-thiourea;
N-(cis-4-{[4-(dimethylamino)quinazolin-2-yl]amino}cyclohexyl)-N′-(pentafluorophenyl)-thiourea;
N-(cis-4-{[4-(dimethylamino)quinazolin-2-yl]amino) cyclohexyl)-N′-propylthiourea;
N-(cis-4-{[4-(dimethylamino)quinazolin-2-yl]aminoll cyclohexyl)-N′-(3,4,5-trimethoxyphenyl)thiourea;
N-(cis-4-{[4-(dimethylamino)quinazolin-2-yl]amino}cyclohexyl)-N′-(4-methylphenyl)-thiourea;
N-(3,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(4-ethylphenyl)-thiourea;
N-(cis-4-{[4-(dimethylamino)quinazolin-2-yl]amino}cyclohexyl)-N′-[2-(methylthio)-phenyl]thiourea;
N-(cis-4-{[4-(dimethylamino)quinazolin-2-yl]amino}cyclohexyl)-N′-[2-(trifluoromethoxy)-phenyl]thiourea;
N-(cis-4-{[4-(dimethylamino)quinazolin-2-yl]amino) cyclohexyl)-N′-(2,3,4-trifltorophenyl)-thiourea;
N-(2,5-dimethoxyphenyl)-N′-(cis-4-{[4-(dimethylamino)quinazolin-2-yl]amino}-cyclohexyl)thiourea;
N-(2-chloro-4-nitrophenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethylphenyl)-thiourea;
N-(cis-4-{[4-(dimethylamino)quinazolin-2-yl]amino}cyclohexyl)-N′-(2-iodophenyl)-thiourea;
N-(cis-4-{[4-(dimethylamino)quinazolin-2-yl]amino}cyclohexyl)-N′-(2-methoxy-4-nitrophenyl)thiourea;
N-(cis-4-{[4-(dimethylamino)quinazolin-2-yl]amino}cyclohexyl)-N′-(2-methoxy-5-methylphenyl)thiourea;
N-(cis-4-}[4-(dimethylamino)quinazolin-2-yl]amino) cyclohexyl)-N′-(3-iodophenyl)-thiourea;
N-(cis-4-{[4-(dimethylamino)quinazolin-2-yl]amino] cyclollexyl)-N′-(3-methoxyphenyl)-thiourea;
N-[4-(difluoromethoxy)phenyl)-N′-(cis-4-{[4-(dimethylamino)quinazolin-2-yl]amino}-cyclohexyl)thiourea;
N-(cis-4-}[4-(dimethylamino)quinazolin-2-yl]amino) cyclohexyl)-N′-[4-(trifluoromethyl)-phenyl]thiourea;
N-(4-bromo-2-chlorophenyl)-N′-(cis-4-}[4-(dmethylamino)quinazolin-2-yl]amino}-cyclohexyl)thiourea;
N-(cis-4-{[4-(dimethylamino)quinazolin-2-yl]amino}cyclohexyl)-N′-(4-iodophenyl)-thiourea;
N-(5-chloro-2-methylphenyl)-N′-(cis-4-{[4-(dimethylamino)quinazolin-2-yl]amino}-cyclohexyl)thiourea;
N-[(1S,4R)-bicyclo[2.2.1]hept-2-yl)-N′-(cis-4-{[4-(dimethylamino)quinazolin-2-yl]amino}-cyclohexyl)thiourea;
N-[2-(4-chlorophenyl)ethyl]-N′-(cis-4-{[4-(dimethylanmino)quinazolin-2-yl]amino}-cyclohexyl)thiourea;
N-(cis-4-{[4-(dimethylamino)quinazolin-2-yl]amino}cyclohexyl)-N′-(2,4,6-tribromophenyl)thiourea;
N-(cis-4-{[4-(dimethylamino)quinazolin-2-yl]amino}cyclohexyl)-N′-N′-(2,4,6-trichlorophenyl)-thiourea;
N-(cis-4-{[4-(dimethylamino)quinazolin-2-yl]amino}cyclohexyl)-N′-mesitylthiourea;
N-(cis-4-{[4-(dimethylamino)quinazolin-2-yl]amino}cyclohexyl)-N′-(2,4-dimethylphenyl)-thiourea;
N-(2,6-diethylphenyl)-N′-(cis-4-{[4-(dimethylamino)quinazolin-2-yl]amino}cyclohexyl)-thiourea;
N-(2,6-diisopropylphenyl)-N′-(cis-4-{[4-(dimethylamino)quinazolin-2-yl]amino}-cyclohexyl)thiourea;
N-(2-bromo-4-methylphenyl)-N′-(cis-4-{[4-(dimethylamino)quinazolin-2-yl]amino}-cyclohexyl)thiourea;
N-(2-clorobenzyl)-N′-(cis-4-{[4-(dimethylamino)quinazolin-2-yl]amino}cyclohexyl)-thiourea;
N-(cis-4-{[4-(dimethylamino)quinazolin-2-yl]amino}cyclohexyl)-N′-(2-ethyl-6-methylphenyl)thiourea;
N-(cis-4-{[4-(dimethylamino)quinazolin-2-yl]amino}cyclolexyl)-N′-(2-isopropylphenyl)-thiourea;
N-(3,5-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(3,5-dimethylphenyl)-thiourea;
N-(3-chloro-4-methylphenyl)-N′-(cis-4-{[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl)}amino)benzoate;
N-(4-bromo-2,6-dimethylphenyl)-N′-(cis-4-{[4-(dimethylamino)quinazolin-2-yl]amino}cyclohexyl)thiourea;
N-(4-bromo-2-methylphenyl)-N′-(cis-4-{[4-(dimethylamino)quinazolin-2-yl]amino}-cyclohexyl)thiourea;
N-[4-bromo-2-(trifluoromethyl)phenyl]-N′-(cis-4-{[4-(dimethylamino)quinazolin-2-yl]amino}cyclohexyl)thiourea;
N-(4-chloro-2-methylphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-[1-(4-fluorophenyl)-ethyl]thiourea;
N-(cis-4-{[4-(dimethylamino)quinazolin-2-yl]amino}cyclohexyl)-N′-(4-fluorobenzyl)-thiourea;
N-(cis-4-{[4-(dimethylamino)quinazolin-2-yl]amino}cyclohexyl)-N′-(4-isopropylphenyl)-thiourea;
N-(cis-4-{[4-(dimethylamino)quinazolin-2-yl]amino}cyclohexyl)-N′-(4-methoxybenzyl)-thiourea;
methyl 4-{[(cis-4-{[4-(dimethylamino)quinazolin-2-yl]amino}cyclohexyl)amino]-carbonothioyl}amino)benzoate;
N-(cis-4-{[4-(dimethylamino)quinazolin-2-yl]amino}cyclohexyl)-N′-(1-phenylethyl)-thiourea;
N-(cis-4-{[4-(dimethylamino)quinazolin-2yl]amino}cyclohexyl)-N′-(diphenylmethyl)-thiourea;
N-(cyclohexylmethyl)-N′-(cis-4-{[4-(dimethylamino)quinazolin-2-yl]amino}cyclolhexyl)-thiourea;
N-cyclooctyl-N′-(cis-4-{[4-(dimethylamino)quinazolin-2-yl]amino}cyclohexyl)thiourea;
N-cyclopropyl-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(1-naphthylmethyl)-thiourea;
N-(cis-4-{[4-(dimethylamino)quinazolin-2-yl]amino}cyclohexyl)-N′-(2,2-diphenylethyl)-thiourea;
N-(2,3-dimethoxybenzyl)-N′-(cis-4-{[4-(dimethylamino)quinazolin-2-yl]amino}cyclohexyl)-thiourea;
N-(cis-4-{[4-(dimethylamino)quinazolin-2-yl]amino}cyclohexyl)-N′-(2,4,5-trimethylphenyl)thiourea;
N-[2-(2,5-dimethoxyphenyl)ethyl]-N′-(cis-4-{[4-(dimethylamino)quinazolin-2-yl]amino}-cyclohexyl)thiourea;
N-biphenyl-2-yl-N′-(cis4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2-fluorobenzyl)-thiourea;
N-(cis-4-{[4-(dimethylamino)quinazolin-2-yl]amino}cyclohexyl)-N′-(2-methyl-4-nitrophenyl)thiourea;
N-(cis-4-{[4-(dimethylamino)quinazolin-2-yl]amino}cyclohexyl)-N′-(2-methylbenzyl)-thiourea;
N-(3-chlorobenzyl)-N′-(cis-4-{[4-(dimethylamino)quinazolin-2-yl]amino}cyclohexyl)-thiourea;
ethyl 3-({[(cis-4-{[4-(dimethylamino)quinazolin-2-yl]amino}cyclohexyl)amino]-carbonothioyl)amino)benzoate;
N-(cis-4-{[4-(dimethylamino)quinazolin-2-yl]amino}cyclohexyl)-N′-(3-ethylpheylyl)-thiourea;
N-(cis-4-{[4-(dimethylamino)quinazolin-2-yl]amino}cyclohexyl)-N′-(3-fluorobenzyl)-thiourea;
N-(cis-4-{[4-(dimethylamino)quinazolin-2-yl]amino}cyclohexyl)-N′-(3-methoxybenzyl)-thiourea;
N-(cis-4-{[4-(dimethylamino)quinazolin-2-yl]amino}cyclohexyl)-N′-(3-methylbenzyl)-thiourea;
N-[4-chloro-2-(trifluoromethyl)phenyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(4-fluoro-2-methylphenyl)thiourea;
N-(cis-4-{[4-(dimethylamino)quinazolin-2-yl]amino}cyclohexyl)-N′-(4-methoxy-2-methylphenyl)thiourea;
N-(5-chloro-2,4-dimethoxyphenyl)-N′-(cis-4-}[4-(dimethylamino)quinazolin-2-yl]amino}-cyclohexyl)thiourea;
N-(2,3-dihydro-1H-inden-5-yl)-N′-(cis-4-{[4-(dimethylamino)quinazolin-2-yl]amino}-cyclohexyl)thiourea;
N-cycloheptyl-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[(1R)-1-phenylethyl]-thiourea;
N-(2-cyclohex-1-en-1-ylethyl)-N′-(cis-4-{[4-(dimetlylamino)quinazolin-2-yl]amino}-cyclohexyl)thiourea;
N-(cis-4-{[4-(dimethylamino)quinazolin-2-yl]amino}cyclohexyl)-N′-(2,3-dimethylphenyl)-thiourea;
N-(2,4-dibromo-6-fluorophenyl)-N′-(cis-4-{[4-(dimethylamino)quinazolin-2-yl]amino}-cyclohexyl)thiourea;
N-(2,4-dichloro-6-methylphenyl)-N′-(cis-4-{[4-(dimethylamino)quinazolin-2-yl]amino}-cyclohexyl)thiourea;
N-(cis-4-{[4-(dimethylamino)quinazolin-2-]amino}cyclohexyl)-N′-(2,5-dimethylphenyl)-thiourea;
N-(2-bromo-4-isopropylphenyl)-N′-(cis-4-{[4-(dimethylamino)quinazolin-2-yl]amino}-cyclohexyl)thiourea;
N-(2-bromo-5-fluorophenyl)-N′-(cis-4-{[4-(dimethylamino)quinazolin-2-yl]amino}-cyclohexyl)thiourea;
N-(cis-4-{[4-(dimethylamino)quinazolin-2-yl]amino) cyclohexyl)-N′-(2-ethoxyphenyl)-thiourea;
N-(cis-4-{[4-(dimethylamino)quinazolin-2-yl]amino}cyclohexyl)-N′-(2-isopropyl-6-methylphenyl)thiourea;
N-(cis-4-{[4-(dimethylamino)quinazolin-2-yl]amino) cyclohexyl)-N′-(2-methoxybenzyl)-thiourea;
N-(2,3-dihydro-1,4-benzodioxin-6-yl)-N′-(cis4-{[4-(dimethylamino)quinazolin-2-yl]amino}cyclohexyl)thiourea;
N-1,3-benzodioxol-5-yl-N′-(cis-4-{[4-(dimethylamino)quinazolin-2-yl]amino}cyclohexyl)-thiourea;
N-(3-chloro-2-methlylphenyl)-N′-(cis-4-{[4-(dimethylamino)quinazolin-2-yl]amino}-cyclohexyl)thiourea;
N-[4-bromo-2-(trifluoromethoxy)phenyl]-N′-(cis-4-{[4-(dimethylamino)quinazolin-2-yl]amino}cyclohexyl)thiourea;
N-(4-chloro-2,5-dimethoxyphenyl)-N′-(cis-4-}[4-(dimethylamino)quinazolin-2-yl]amino}-cyclohexyl)thiourea;
N-(cis-4-{[4-(dimethylamino)quinazolin-2-yl]amino}cyclohexyl)-N′-(4-phenylbutyl)-thiourea;
N-bicyclo[2.2.1]hept-2-yl-N′-(cis-4-[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl}amino)-4-methylthiophene-2-carboxylate;
methyl 3-({[(cis-4-{[4-(dimethylamino)quinazolin-2-yl]amino}cyclohexyl)amino]-carbonothioyl}amino)thiophene-2-carboxylate;
N-(2-bromo-4-fluorophenyl)-N′-(cis-4-{[4-(dimethylamino)quinazolin-2-yl]amino}-cyclohexyl)thiourea;
N-(4-butyl-2-methylphenyl)-N′-(cis-4-{[4-(dimetlhylamino)quinazolin-2-yl]amino}-cyclohexyl)thiourea;
N-[4-(dimethylamino)-1-naphthyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(5-methyl-3-phenylisoxazol-4-yl)thiourea;
N-[(cis-4-{[4-(dimethylamino)quinazolin-2-yl]amino}cyclohexyl)methyl]-N′-(2,6-dimethylphenyl)thiourea;
N-(2,6-dichlorophenyl)-N′-[(cis-4-{[4-(dimethylamino)quinazolin-2-yl]amino}cyclohexyl)-methyl]thiourea;
N-[(cis-4-{[4-(methylamino)quinazolin-2-yl]amino) cyclohexyl)methyl]-N′-(2-ethyl-6-isopropylphenyl)thiourea;
N-[(cis-4-{[4-(dimethylamino)quinazolin-2-yl]amino) cyclohexyl)methyl]-N′-isobutylthiourea;
N-(1,3-benzodioxol-5-ylmethyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(4-nitrophenyl)thiourea;
N-[(cis-4-{[4-(dimethylamino)quinazolin-2-yl]amino}cyclohexyl)methyl)]-N′-(pentafluorophenyl)thiourea;
N-[(cis-4-{[4-(dimethylamilo)quinazolin-2-yl]amino}cyclohexyl)methyl]-N′-(tetrahydrofuran-2-ylmethyl)thiourea;
N-[(cis-4-{[4-(dimethylamino)quinazolin-2-yl]amino}cyclohexyl)methyl]-N′-[2-(trifluoromethoxy)phenyl]thiourea;
N-[(cis-4-{[4-(dimethylamino)quinazolin-2-yl]amine}cyclohexyl)methyl]-N′-(2,3,4-trifluorophenyl)thiourea;
N-[(cis-4-{[4-(dimethylamino)quinazolin-2-yl]amino}cyclohexyl)methyl]-N′-(2-ethylphenyl)thiourea;
N-(5-chloro-2-methylphenyl)-N′-[(cis-4-{[4-(dimethylamino)quinazolin-2-yl]amino}-cyclohexyl)methyl)thiourea;
N-[(1S,4R)-bicyclo[2.2.1]hept-2-yl]-N′-[(cis-4-{[4-(dimethylamino)quinazolin-2-yl]amino}cyclohexyl)methyl]thiourea;
N-[2-(3,4-methoxyphenyl)ethyl]-N′-[(cis-4-{[4-(dimethylamino)quinazolin-2-yl]amino}-cyclohexyl)methyl]thiourea;
N-[(cis-4-{[4-(dimethylamino)quinazolin-2-yl]amino}cyclohexyl)methyl]-N′-(2,4,6-tribromophenyl)thiourea;
N-[(cis-4-{[4-(dimethylamino)quinazolin-2-yl]amino}cyclohexyl)methyl]-N′-(2,4,6-trichlorophenyl)thiourea;
N-[(cis-4-{[4-(dimethylamino)quinazolin-2-yl]amino}cyclohexyl)metiyl]-N′-mesitylthiourea;
N-(2,6-diethylphenyl)-N′-[(cis-4-{[4-(dimethylamino)quinazolin-2-yl]amino}cyclohexyl)-methyl)thiourea;
N-(2,6-diisopropylphenyl)-N′-[(cis-4-{[4-(dimethylamino)quinazolin-2-yl]amino}-cyclohexyl)methyl]thiourea;
N-[(cis-4-{[4-(dimethylamino)quinazolin-2-yl]amino}cyclohexyl)methyl]-N′-(2-ethyl-6-methylphenyl)thiourea;
N-[(cis-4-{[4-(dimethylamino)quinazolin-2yl-amino}cyclohexyl)methyl)]-N′-(2-isopropylphenyl)thiourea;
N-(4-bromo-2,6-dimethylphenyl)-N′-[(cis-4-{[4-(dimethylamino)quinazolin-2-yl]amino}-cyclohexyl)methyl]thiourea;
N-[4-bromo-2-(trifluoromethyl)phenyl]-N′-[(cis-4-{[4-(dimethylamino)quinazolin-2-yl]amino}cyclobexyl)methyl]thiourea;
N-[(cis-4-{[4-(dimethylamino)quinazolin-2-yl]amino}cyclohexyl)methyl]-N′-[1-(4-fluorophenyl)ethyl]thiourea;
N-(5-chloro-2-methoxyphenyl)-N′-[(cis-4-{[4-(dimethylamino)quinazolin-2-yl]amino}-cyclohexyl)methyl]thiourea;
N-[(cis-4-{[4-(dimethylamino)quinazolin-2-yl]amino}cyclohexyl)methyl]-N′-(diphenylmnethyl)thiourea;
N-cyclododecyl-N′-[(cis-4-{[4-(dimethylamino)quinazolin-2-yl]amino}cyclohexyl)methyl]-thiourea;
N-(cyclohexylmethyl)-N′-[(cis-4-{[4-(dimethylamino)quinazolin-2-yl]amino}cyclohexyl)-methyl]thiourea;
N-[(cis-4-{[4-(dimethylamino)quinazolin-2-yl]amino}cyclohexyl)methyl]-N′-(2,3,5,6-tetrachlorophenyl)thiourea;
N-(2,3-dimethoxybenzyl)-N′-[(cis-4-{[4-(dimethylamino}quinazolin-?-yl]amino}-cyclohexyl)methyl]thiourea;
N-(2,4-dichlorobenzyl)-N′-[(cis-4-{[4-(dimethylamino}quinazolin-2-yl]amino}cyclohexyl)-methyl]thiourea;
N-[(cis-4-{[4-(dimethylamino)quinazolin-2-yl]amino}cyclohexyl)methyl]-N′-(2-methoxy-5-nitrophenyl)thiourea;
N-[(cis-4-{[4-(dimethylamino)quinazolin-2-yl]amino}cyclohexyl)methyl]-N′-(4-metl)oxy-2-methylphenyl)thiourea;
N-(2,4-dibromo-6-fluorophenyl)-N′-[(cis-4-{[4-(dimethylamino)quinazolin-2-yl]amino}-cyclohexyl)methyl]thiourea;
N-(2,4-dichloro-6-methylphenyl)-N′-[(cis-4-{[4-(dimethylamino)quinazolin-2-yl]amino}-cyclohexyl)methyl]thiourea;
N-[(cis-4-{[4-(dimethylamino)quinazolin-2-yl]amino}cyclohexyl)methyl]-N′-(2,5-dimethylphenyl)thiourea;
N-[(cis-4-{[4-(dimethylamino)quinazolin-2-yl]amino}cyclohexyl)methyl]-N′-(2-ethoxyphenyl)thiourea;
N-[(cis-4-{[4-(dimethylamino)quinazolin-2-yl]amino}cyclohexyl)methyl]-N′-(2-isopropyl-6-methylphenyl)thiourea;
N-[4-bromo-2-(trifluoromethoxy)phenyl]-N′-[(cis-4-{[4-(dimethylamino)quinazolin-2-yl]amino}cyclohexyl)methyl]thiourea;
N-bicyclo[2.2.1]hept-2-yl-N′-[(cis-4-{[4-(dimethylamino)quinazolin-2-yl]amino}-cyclohexyl)methyl]thiourea;
N-bicyclo[2.2.1]hept-5-en-2-yl-N′-[(cis-4-{[4-(dimethylamino)quinazolin-2-yl]amino}-cyclohexyl)methyl]thiourea;
N-(cyclopropylmethyl)-N′-[(cis-4-{[4-(dimethylamino)quinazolin-2-yl]amino}cyclohexyl)-methyl]thiourea; and
N-[(cis-4-{[4-(dimethylamino)quinazolin-2-yl]amino}cyclohexyl)methyl]-N′-(5-methyl-3-phenylisoxazol-4-yl)thiourea;
   or a pharmaceutically acceptable salt, hydrate or solvate thereof.

In some embodiments compounds of the present invention are of Formula (I) wherein the compound is selected from the group consisting of:
N-(4-bromopheinyl)-N′-(cis-4-{[4-(dimethylamino)quinazolin-2-yl]amino}cyclohexyl)-thiourea;
N-(4-cyanophenyl)-N′-(cis-4-{[4-(dimethylamino)quinazolin-2-yl]amino}cyclohexyl)-thiourea;
N-(2,4-dichlorophenyl)-N′-(cis-4-{[4-(dimethylamino)quinazolin-2-yl]amino}cyclohexyl)-thiourea;
N-(2,4-dimethoxyphenyl)-N′-(cis-4-{[4-(ditnethylamino)quinazolin-2-yl]amino}-cyclohexyl)thiourea;
N-(cis-4-{[4-(dimethylamino)quinazolin-2-yl]amino}cyclohexyl)-N′-(2,6-dimethylphenyl)-thiourea;
N-(cis-4-{[4-(dimethylamino)quinazolin-2-yl]amino}cyclohexyl)-N′-(2-ethyl-6-isopropylphenyl)thiourea;
N-(cis-4-{[4-(dimethylamino)quinazolin-2-yl]amino}cyclohexyl)-N′-(-2-methoxyphenyl)-thiourea;
N-(cis-4-{[4-(dimethylamino)quinazolin-2-yl]amino}cyclohexyl)-N′-1-naphthylthiourea;
N-(cis-4-{[4-(dimethylamino)quinazolin-2-yl]amino}cyclohexyl)-N′-(3,4,5-trimethoxyphenyl)thiourea;
N-(3,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethylphenyl)-thiourea;
N-(cis-4-{[4-(dimethylamino)quinazolin-2-yl]amino}cyclohexyl)-N′-(2-methoxy-4-nitrophenyl)thiourea;
N-(cis-4-{[4-(dimethylamino)quinazolin-2-yl]amino}cyclohexyl)-N′-(2-methoxy-5-methylphenyl)thiourea;
N-(4-bromo-2-chlorophenyl)-N′-(cis-4-{[4-(dimethylaimino)quinazolin-2-yl]amino}-cyclohexyl)thiourea;
N-(cis-4-{[4-(dimethylamino)quinazolin-2-yl]amino}cyclohexyl)-N′-(4-iodophenyl)-thiourea;
N-(cis-4-{[4-(dimethylamino)quinazolin-2-yl]amino}cyclohexyl)-N′-(2,4,6-tribromophenyl)thiourea;
N-(cis-4-{[4-(dimethylamino)quinazolin-2-yl]amino}cyclolhexyl)-N′-(2,4,6-trichlorophenyl)-thiourea;
N-(cis-4-{[4-(dimetlylamino)quinazolin-2-yl]amino}cyclohexyl)-N′-mesitylthiourea;
N-(cis-4-{[4-(dimethylamino)quinazolin-2-yl]amino}cyclohexyl)-N′-(2,4-dimethylphenyl)-thiourea;
N-(2,6-diethylphenyl)-N′-(cis-4-{[4-(dimethylamino)quinazolin-2-yl]amino}cyclohexyl)-thiourea;
N-(2-bromo-4-methylphenyl)-N′-(cis-4-{[4-(dimethylamino)quinazolin-2-yl]amino}-cyclohexyl)thiourea;
N-(2-chlorobenzyl)-N′-(cis-4-{[4-(dimethylamino)quinazolin-2-yl]amino}cyclohexyl)-thiourea;
N-(cis-4-{[4-(dimethylamino)quinazolin-2-yl]amino}cyclohexyl)-N′-(2-ethyl-6-methylphenyl)thiourea;
N-(cis-4-{[4-(dimethylamino)quinazolin-2-yl]amino}cyclohexyl)-N′-(2-isopropylphenyl)-thiourea;
methyl 3-({[(cis-4-{[4-(dimethylamino)quinazolin-2-yl]amino}cyclohexyl)amino]-carbonothioyl}amino)benzoate;
N-(4-bromo-2,6-dimethylphenyl)-N′-(cis-4-{[4-(dimethylamino)quinazolin-2-yl]amino}cyclohexyl)thiourea;
N-(4-bromo-2-methylphenyl)-N′-(cis-4-{[4-(dimethylamino)quinazolin-2-yl]amino}-cyclohexyl)thiourea;
N-[4-bromo-2-(trifluoromethyl)phenyl]-N′-(cis-4-{[4-(dimethylamino)quinazolin-2-yl]amino}cyclohexyl)thiourea;
N-(4-chioro-2-methylphenyl)-N′-(cis-4-{[4-(dimetlylamino)quinazolin-2-yl]amino}-cyclohexyl)thiourea;
N-(cis-4-{[4-(dimethylamino)quinazolin-2-yl]amino}cyclohexyl)-N′-(1-naphthylmethyl)-thiourea;
N-(2,3-dimetihoxybenzyl)-N′-(cis-4-{[4-(dimethylaniiiio)quinazolin-2-yl]amino}cyclohexyl)-thiourea;
N-(cis-4-{[4-(dimethylamino)quinazolin-2-yl]amino}cyclohexyl)-N′-(2,4,5-trimethylphenyl)thiourea;
N-biphenyl-2-yl-N′-(cis-4-{[4-(dimethylamino)quinazolin-2-yl]amino}cyclohexyl)thiourea;
N-(cis4-{[4-(dimethylamino)quinazolin-2-yl]amino}cyclohexyl)-N′-(2-methyl-4-nitrophenyl)thiourea;
N-(3-chlorobenzyl)-N′-(cis-4-{[4-(dimethylamino)qtuiinazolin-2-yl]amino}cyclohexyl)-thiourea;
ethyl 3-({[(cis-4-{[4-(dimethylamino)quinazolin-2-yl]amino}cyclohexyl)amino]-carbonothioyl)amino)benzoate;
N-[4-chloro-2-(trifluoromethyl)phenyl)-N′-(cis-4-{[4-(dimetlylamino)quinazolin-2-yl]amino}cyclohexyl)thiourea;
N-(cis-4-{[4-(dimethylamino)quinazolin-2-yl]amino}cyclohexyl)-N′-(4-fluoro-2-methylphenyl)thiourea;
N-(cis-4-{[4-(dimethylamino)quinazolin-2-yl]amino) cyclohexyl)-N′-(4-methoxy-2-methylphenyl)thiourea;
N-(5-chloro-2,4-dimethoxyphenyl)-N′-(cis-4-{[4-(dimethylamino)quinazolin-2-yl] amino}-cyclohexyl)thiourea;
N-(cis-4-{[4-(dimethylamino)quinazolin-2-yl]amino}cyclohexyl)-N′-[(1R)-1-phenylethyl]-thiourea;
N-(cis-4-{[4-(dimethylamino)quinazolin-2-yl]amino) cyclohexyl)-N′-(2,3-dimethylphenyl)-thiourea;
N-(2,4-dibromo-6-fluorophenyl)-N′-(cis-4-{[4-(dimethylamino)quinazolin-2-yl]amino}-cyclohexyl)thiourea;
N-(2,4-dichloro-6-methylphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethoxyphenyl)-thiourea;
N-(cis-4-{[4-(dimethylamino)quinazolin-2-yl]amino}cyclohexyl)-N′-(2-isopropyl-6-methylphenyl)thiourea;
N-(2,3-dihydro-1,4-benzodioxin-6-yl)-N′-(cis-4-{[4-(dimethylamino)quinazolin-2-yl]amino}cyclohexyl)thiourea;
N-1,3-benizodioxol-5-yl-N′-(cis-4-{[4-(dimethylamino)quinazolin-2-yl]amino}cyclolhexyl)-thiourea;
N-(3-chloro-2-methylphenyl)-N′-(cis-4-{[4-(dimethylamino)quinazolin-2-yl]amino}-cyclohexyl)thiourea;
N-[4-bromo-2-(trifluoromethoxy)phenyl]-N′-(cis-4-{[4-(dimethylamino)quinazolin-2-yl]amino}cyclohexyl)thiourea;
N-(4-chloro-2,5-dimethoxyphenyl)-N′-(cis-4-{[4-(dimethylamino)quinazolin-2-yl]amino}-cyclohexyl)thiourea;
N-bicyclo[2.2.1]hept-2-yl-N′-(cis-4-{[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl}amino)-4-methylthiophene-2-carboxylate;
methyl 3-({[(cis-4-{[4-(dimethylamino)quinazolin-2-yl]amino}cyclohexyl)amino]-carbonothioyl}amino)thiophene-2-carboxylate;
N-(4-butyl-2-methylphenyl)-N′-(cis-4-{[4-(dimethylamino)quinazolin-2-yl]amino}-cyclohexyl)thiourea;
N-[4-(dimethylamino)-1-naphthyl]-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(5-methyl-3-phenylisoxazol-4-yl)thiourea;
N-(2,6-diethylphenyl)-N′-[(cis-4-{[4-(dimethylamino)quinazolin-2-yl]amino}cyclohexyl)-methyl]thiourea;
N-(4-bromo-2,6-dimethylphenyl)-N′-[(cis-4-{[4-(dimethylamino)quinazolin-2-yl]amino}-cyclohexyl)methyl]thiourea;
N-[(cis-4-{[4-(dirnetiylamino)quinazolin-2-yl]amino}cyclohexyl)methyl]-N′-(2,3,5,6-tetrachlorophenyl)thiourea; and
N-[(cis-4-{[4-(dimethylamino)quinazolin-2-yl]amino) cyclohexyl)methyl]-N′-(2-isopropyl-6-methylphenyl)thiourea;
   or a pharmaceutically acceptable salt, hydrate or solvate thereof.

In some embodiments of the present invention, R₁ is selected from the group consistihg of:
R₁ is selected from the group consisting of:
(i) C₁₋₈ alkyl, and C₁₋₈ alkyl substituted by substituent(s) independently selected from the group consisting of:
   halogen
   C₁₋₅ alkoxy,
   C₁₋₅ alkoxy substituted by carbocyclic aryl,
   carbocyclyl,
   carbocyclic aryl,
   carbocyclic aryl substituted by substituent(s) independently selected from the group consisting of:
      halogen,
      nitro, and
   C₁₋₅ alkoxy,
(ii) C₂₋₅ alkenyl,
(iii) carbocyclyl,
(iv) carbocyclic aryl, and carbocyclic aryl substituted by substituent(s) independently selected from the group consisting of:
   halogen,
   C₁₋₅ alkyl,
   C₁₋₅ alkyl substituted by halogen, and
   C₁₋₅ alkoxy;
   L is Formula (V); and
   Y is —C(O)O—;
   wherein carbocyclic aryl is phenyl or naphthyl;
   carbocyclyl is 9H-fluorenyl or menthyl; and
   halogen is fluoro, chloro, bromo, or iodo;
or a pharmaceutically acceptable salt, hydrate or solvate thereof.

In some embodiments of the present invention, R₄ₐ is hydrogen or methyl; R_{4b} is methyl; R₅ and R₆ are hydrogen; A is a single bond; and B is a single bond or —CH₂—; or a pharmaceutically acceptable salt, hydrate or solvate thereof.

In some embodiments, compounds of the present invention are of Formula (I) wherein the compound is selected from the group consisting of:
cis-[4-(4-dimethylamino-quinazolin-2-ylamino)-cyclohexyl]-carbamic acid 2-benzyloxy-ethyl ester;
cis-[4-(4-dimethylamino-quinazolin-2-ylamino)-cyclohexyl]-carbamic acid 4,5-dimethoxy-2-nitro-benzyl ester;
cis-[4-(4-dimethylamino-quinazolin-2-ylamino)-cyclohexyl]-carbamic acid 2-chloro-benzyl ester;
cis-[4-(4-dimethylamino-quinazolin-2-ylamino)-cyclohexyl]-carbamic acid 4,5-dimethoxy-2-nitro-benzyl ester;
cis-[4-(4-dimethylamino-quinazolin-2-ylamino)-cyclohexyl]-carbamic acid 4-nitro-benzyl ester;
cis-[4-(4-dimethylamino-quinazolin-2-ylamino)-cyclohexyl]-carbamic acid benzyl ester;
cis-[4-(4-dimethylamino-quinazolin-2-ylamino)-cylohexylmethyl]-carbamic acid 2-chloro-benzyl ester;
cis-[4-(4-dimetlylamino-quinazolin-2-ylamino)-cyclohexylmethyl]-carbamic acid 4-nitro-benzyl ester; and
cis-[4-(4-dimethylamino-quinazolin-2-ylamino)-cyclohexylmethyl]-carbamic acid benzyl ester;
   or a phannaceutically acceptable salt, hydrate or solvate thereof.

In some embodiments of the present invention, R₁ is C₁₋₈ alkyl, and
C₁₋₈ alkyl substituted by substituent(s) independently selected from the group consisting of:
   carbocyclic aryl,
   carbocyclic aryl substituted by substituent(s) independently selected from the group consisting of:
      halogen,
      C₁₋₅ alkyl,
      C₁₋₅alkyl substituted by halogen,
      C₁₋₅ alkoxy, and
      C₁₋₅ alkoxy substituted by halogen,
R₄ is —N(R₄ₐ)(R_{4b}) wherein R₄ₐ and R_{4b} are independently C₁₋₅ alkyl;
L is Formula (III) or (IX) wherein R₅ and R₆ are both hydrogen; A and B are each independently a single bond or —CH₂—; and
Y is a single bond;
wherein carbocyclic aryl is phenyl; and
halogen is fluoro or chloro;
or a pharmaceutically acceptable salt, hydrate or solvate thereof.

In some embodinments of the present invention, R₁ is C₁₋₈ alkyl, and
C₁₋₈ alkyl substituted by substituent(s) independently selected from the group consisting of:
   carbocyclic aryl,
   carbocyclic aryl substituted by substituent(s) independently selected from the group consisting of:
      C₁₋₅ alkoxy, and
      C₁₋₅ alkoxy substituted by halogen,
wherein carbocyclic aryl is phenyl; and
halogen is fluoro or chloro;
or a phan-naceutically acceptable salt, hydrate or solvate thereof

In some embodiments of the present invention, R₄ is —N(CH₃)₂; L is Formula (VIII) or (IX) wherein A is a single bond and B is —CH₂—, or A is —CH₂— and B is a single bond; and Y is a single bond; wherein carbocyclic aryl is phenyl; and halogen is fluoro; or a pharmaceutically acceptable salt, hydrate or solvate thereof.

In some embodiments, compounds of the present invention are of Formula (I) wherein the compound is selected from the group consisting of:
N²-[(1S,3R)-3-({[4-bromo-2-(trifluoromethoxy)benzyl]amino}-methyl)cyclopentyl]-N¹,N⁴-dimethylquinazoline-2,4-diamine;
or a pharmaceutically acceptable salt, hydrate or solvate thereof.

In some embodiments of the present invention, R₁ is selected from the group consisting of:
(i) C₁₋₈ alkyl, and C₁₋₈ alkyl substituted by substittlent(s) independently selected from the group consisting of:
   carbocyclic aryl,
   carbocyclic aryl substituted by substituent(s) independently selected from the group consisting of:
      hydroxy,
      halogen,
      nitro,
      C₁₋₅ alkylcarbonylamino,
      C₃₋₆ cycloalkylcarbonylamino,
      C₁₋₅ alkyl,
      C₁₋₅ alkyl substituted by halogen,
      C₁₋₅ alkylsulfonyl,
      C₁₋₅ alkoxy,
      C₁₋₅ alkoxy substituted by halogen, and carbocyclic aryl,
   heterocyclyl, and
   heterocyclyl substituted by halogen,
(ii) C₃₋₁₂ cycloalkyl, and C₃₋₁₂ cycloalkyl substituted by carbocyclic aryl,
(iii) carbocyclyl, and carbocyclyl by substittent(s) independently selected from the group consisting of:
   hydroxy, and
   carbocyclic aryl,
(iv) carbocyclic aryl, and carbocyclic aryl substituted by substituent(s) independently selected from the group consisting of:
   halogen,
   C₁₋₅ alkoxy, and
   nitro,
(v) heterocyclyl, and heterocyclyl substituted by substituent(s) independently selected from the group consisting of:
   halogen, and
   C₁₋₅ alkoxy,
R₄ is —N(R₄ₐ)(R_{4b}) wherein R₄ₐ and R_{4b} are each independently C₁₋₅ alkyl;
L is Formula (XIII); wherein R₅ and R₆ are both hydrogen; A is a single bond and B is a single bond or —CH₂—; and
Y is —C(O)NR₇—, wherein R₇ is hydrogen or C₁₋₅ alkyl;
   wherein carbocyclic aryl, is phenyl or naphthyl;
   carbocyclyl is indanyl, 9H-fluorenyl, 1,2,3,4-tetralhydro-naphthalen-1-yl, or 1H-indolyl;
   heterocyclyl is benzo[1,3]dioxolyl, pyridyl, dibenzofuranyl, 1H-benzoimidazolyl, or thiazolyl; and
   halogen is fluoro, chloro, bromo, or iodo;
or a pharmaceutically acceptable salt, hydrate or solvate thereof.

In some embodiments of the present invention, R₁ is selected from the group consisting of:
(i) C₁₋₈ alkyl, and C₁₋₈ alkyl substituted by substituent(s) independently selected from the group consisting of:
   carbocyclic aryl,
   carbocyclic aryl substituted by substituent(s) independently selected from the group consisting of:
      hydroxy,
      halogen,
      nitro,
      ₁₋₅ alkylcarbonylamino,
      C₁₋₅ alkyl,
      C₁₋₅ alkyl substituted by halogen,
      C₁₋₅ alkylsulfonyl,
      C₁₋₅ alkoxy,
      C₁₋₅ alkoxy substituted by halogen, and
      carbocyclic aryl,
   hleterocyclyl, and
   heterocyclyl substituted by halogen,
(ii) C₃₋₁₂ cycloalkyl, and C₃-₁₂ cycloalkyl substituted by carbocyclic aryl,
(iii) carbocyclyl,
(iv) carbocyclic aryl, and carbocyclic aryl substituted by substituent(s) independently selected from the group consisting of:
   halogen, and
   nitro,
(v) heterocyclyl, and heterocyclyl substituted by substituent(s) independently selected from the group consisting of:
   halogen, and
   C₁₋₅ alkoxy,
   wherein carbocyclic aryl is phenyl or naphthyl;
   carbocyclyl is indanyl, 9H-fluorenyl, or 1,2,3,4-tetrahydro-naphthalen-1-6l;
   heterocyclyl is benzo[1,3]dioxolyl, or pyridyl; and
   halogen is fluoro, chloro, bromo, or iodo;
or a pharmacetically acceptable salt, hydrate or solvate thereof.

In some embodiments of the present invention, R₄ is —N(CH₃)₂; A and B are both a single bond; and Y is —C(O)NH—; or a pliarmaceutically acceptable salt, hydrate or solvate thereof.

In some embodiments, compounds of the present invention are of Formula (I) wherein the compound is selected from the group consisting of:
cis-4-{[4-(dimethylamino)quinazolin-2-yl]amino}-N-(2,3-dimethylbenzyl)-cyclohexanecarboxamide;
cis-N-(2-bromobenzyl)-4-((4-(dimethylamino)quinazolin-2-yl]amino3-cyclohexanecarboxamide;
cis-N-(2-chlorobenzyl)-4-{[4-(dimethylamino)quinazolin-2-yl]amino}-cyclohexanecarboxamide;
cis-4-{[4-(dimethylamino)quinazolin-2-yl]amino)-N-(4-methylbenzyl)-cyclohexanecarboxamide;
cis-N-[3,5-bis(trifluoromethyl)benzyl]-4-{[4-(dimethylamino)quinazolin-2-yl]amino}-cyclohexanecarboxamide;
cis-N-(2,4-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-(1,2,3,4-tetrahydronaphthalen-1-yl)cyclohexanecarboxamide;
cis-N-(2,3-dihydro-1H-inden-2-yl)-4-{[4-(dimethylamino)quinazolin-2-yl]amino}-cyclohexanecarboxamide;
cis-4-{[4-(dimethylamino)quinazolin-2-yl]amino}-N-[(1S)-1-(4-nitrophenyl)ethyl]-cyclohexanecarboxamide;
cis-N-(3,5-dichlorobenzyl)-4-{[4-(dimethylamino)quinazolin-2-yl]amino}-cyclohexanecarboxamide;
cris-4-{[4-(dimethylamino)quinazolin-2-yl]amino}-N-[4-(trifluoromethoxy)benzyl]-cyclohexanecarboxamide;
cis-N-(4-bromobenzyl)-4-{[4-(dimethylamino)quinazolin-2-yl]amino}-cyclohexanecarboxamide;
cis-4-{[4-(dimethylamino)quinazolin-2-yl]amino}-N-(4-methoxybenzyl)-cyclohexanecarboxamide;
cis-4-{[4-(dimethylamino)quinazolin-2-yl]amino}-N-(2-fluoro-4-nitrophenyl)-cyclohexanecarboxamide;
cis-4-{[4-(dimethylamino)quinazolin-2-yl]amino}-N-(3-fluoro-4-methylbenzyl)-cyclohexanecarboxamide;
cis-N-(5-chloro-2-methylbenzyl)-4-{[4-(dimethylamino)quinazolin-2-yl]amino}-cyclohexanecarboxamide; and
cis-N-(2,4-dichloro-6-methylbenzyl)-4-{[4-(dimethylamino)quinazolin-2-yl]amino}-cyclohexanecarboxamide;
   or a pharmaceutically acceptable salt, hydrate or solvate thereof.

In some embodiments, compounds of the present invention are of Formula (I) wherein the compound is selected from the group consisting of:
cis-N-(2,3-dimethoxybenzyl)-4-{[4-(dimethylamino)quinazolin-2-yl]amino}-cyclohexanecarboxamide;
cis-N-(2,4-difluorobenzyl)-4-{[4-(dimethylamino)quinazolin-2-yl]amino}-cyclohexanecarboxaniide;
cis-N-(2,4-dichlorobenzyl)-4-}[4-(dimethylamino)quinazolin-2-yl]amino}-cyclohexanecarboxamide;
cis-N-(2,3-dichlorobenzyl)-4-{[4-(dimethylamino)quinazolin-2-yl]amino}-cyclohexanecarboxamide;
cis-N-(2,5-dichlorobenzyl)-4-{[4-(dimethylamino)quinazolin-2-yl]amino}-cyclohexanecarboxamide;
cis-N-(33-chlorobenzyl)-4-{[4-(dimethylamino)quinazolin-2-yl]amino}-cyclohexanecarboxamide;
cis-4-{[4-(dimethylamino)quinazolin-2-yl]amino}-N-(3-methoxybenzyl)-cyclohexanecarboxamide;
cis-N-(3,4-dimethoxybenzyl)-4-}[4-(dimethylamino)quinazolin-2-yl]amino}-cyclolhexanecarboxamide;
cis-N-(3,5-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-(4-hydroxy-3-methoxybenzyl)-cyclohexanecarboxamide
cis-N-(1,3-benzodioxol-5-ylmetlhyl)-4-{[4-(dimethylamino)quinazolin-2-yl]amino}-cyclohexanecarboxamide;
cis-4-{[4-(dimethylamino)quinazolin-2-yl]amino}-N-[(1R)-1-(4-nitrophenyl)ethyl]-cyclohexanecarboxamide;
cis-4-(4-dimethylamino-quinazolin-2-ylamino)-cyclohexanecarboxylic acid (trans-2-phenylcyclopropyl)-amide;
cis-4-{[4-(dimethylamino)quinazolin-2-yl]amino}-N-[(1S)-1-(4-methylphenyl)ethyl]-cyclohexanecarboxamide;
cis-4-{[4-(dimethylamino)quinazolin-2-yl]amino}-N-[(1R)-1-(1-naphthyl)ethyl]-cyclohexanecarboxamide;
cis-4-{[4-(dimethylamino)quinazolin-2-yl]amino}-N-[3-(trifluoromethyl)benzyl]-cyclohexanecarboxamide;
cis-4-{[4-(dimethylamino)quinazolin-2-yl]amino}-N-(3-methoxyphenyl)-cyclohexanecarboxamide;
cis-4-{[4-(dimethylamino)quinazolin-2-yl]amino}-N-(3-iodobenzyl)-cyclohexanecarboxamide;
cis-4-{[4-(dimethylamino)quinazolin-2-yl]amino}-N-(4-methoxybenzyl)-cyclohexanecarboxamide;
cis-4-{[4-(dimethylamino)quinazolin-2-yl]amino}-N-(3-iodophenyl)-cyclohexanecarboxamide;
cis-4-{[4-(dimethylamino)quinazolin-2-yl]amino}-N-[3-(propionylamino)benzyl]-cyclohexanecarboxamide;
cis-N-benzyl-4-{[4-(dimethylamino)quinazolin-2-yl]amino}cyclohexanecarboxamide;
cis-N-[(6-chloropyridin-3-yl)methyl]-4-{[4-(dimethylamino)quinazolin-2-yl]amino}-cyclohexanecarboxamide;
cis-4-{[4-(dimethylamino)quinazolin-2-yl]amino}-N-[(1R)-1-(3-mnethoxyphenyl)ethyl]-cyclohexanecarboxamide;
cis-4-{[4-(dimethylamino)quinazolin-2-yl]amino}-N-[1-(4-fluorophenyl)ethyl]-cyclohexanecarboxamide:
cis-N-[(1R)-1-(4-chlorophenyl)ethyl]-4-{[4-(dimethylamino)quinazolin-2-yl]amino}-cyclohexanecarboxamide;
cis-N-[1-(4-bromophenyl)ethyl]-4-{[4-(dimethylamino)quinazolin-2-yl]amino}-cyclohexanecarboxamide;
cis-4-{[4-(dimethylamino)quinazolin-2-yl]amino}-N-[(1S)-1-(1-naphthyl)ethyl]-cyclohexanecarboxamide;
cis-4-{[4-(dimethylamino)quinazolin-2-yl]amino}-N-(3,5-dimethylbenzyl)-cyclohexanecarboxamide:
cis-N-(3-chloro-2-methylbenzyl)-4-{[4-(dimethylamino)quinazolin-2-yl]amino}-cyclohexanecarboxamide;
cis-4-{[4-(dimethylamino)quinazolin-2-yl]amino}-N-(5-fluoro-2-methylbenzyl)-cyclohexanecarboxamide:
cis-N-(3-chloro-2,6-difluorobenzyl)-4-{[4-(dimethylamino)quinazolin-2-yl]amino}-cyclohexanecarboxamide;
cis-N-(biphenyl-3-ylmethyl)-4-{[4-(dimethylamino)quinazolin-2-yl]amino}-cyclohexanecarboxamide;
cis-N-(biphenyl-4-ylmethyl)-4-{[4-(dimethylamino)quinazolin-2-yl]amino}-cyclohexanecarboxamide;
cis-N-(6-chloro-2-fluoro-3-methylbenzyl)-4-{[4-(dimethylamino)quinazolin-2-yl]amino}-cyclohexanecarboxamide;
cis-4-{[4-(dimethylamino)quinazolin-2-yl]amino}-N-(2-fluorobenzyl)-cyclohexanecarboxamide;
cis-N-(2,6-difluorobenzyl)-4-{[4-(dimethylamino)quinazoin-2-yl]amino}-cyclohexanecarboxamide;
cis-4-{[4-(dimethylamino)quinazolin-2-yl]amino}-N-[4-(trifluoromethyl)benzyl]-cyclohexanecarboxamide;
cis-4-{[4-(dimethylamino)quinazolin-2-yl]amino}-N-(1-naphthylmethyl)-cyclohexanecarboxamide;
cis-N-(4-chlorobenzyl)-4-{[4-(dimethylamino)quinazolin-2-yl]amino}-cyclohexanecarboxamide;
cis-N-(3,4-dichlorobenzyl)-4-{[4-(dimethylamino)quinazolin-2-yl]amino}-cyclohexanecarboxamide;
cis-4-{[4-(dimethylamino)quinazolin-2-yl]amino}-N-(3-fluorobenzyl)-cyclohexanecarboxamide;
cis-N-(2,5-difluorobenzyl)-4-{[4-(dimethylamino)quinazolin-2-yl]amino}-cyclohexanecarboxamide;
cis-N-(2,3-difluorobenzyl)-4-{[4-(dimethylamino)quinazolin-2-yl]amino}-cyclohexanecarboxamide;
cis-N-(3-bromobenzyl)-4-{[4-(dimethylamino)quinazolin-2-yl]amino}-cyclohexanecarboxamide;
cis-N-(3-bromo-4-fluorobenzyl)-4-}[4-(dimethylamino)quinazolin-2-yl]amino}-cyclohexanecarboxamide;
cis-N-(4-bromo-2-fluorobenzyl)4-{[4-(dimethylamino)quinazolin-2-yl]amino}-cyclohexanecarboxamide;
cis-N-(5-bromo-2-fluorobenzy)-{[4-(dimethylamino)quinazolin-2-yl]amino)-cyclohexanecarboxamide:
cis-N-(4-chloro-2-fluorobenzyl)-4-[4-(dimethylamino)quinazolin-2-yl]amino}-cyclohexanecarboxamide;
cis-4-{(4-(dimethylamino)quinazolin-2-yl]amino}-N-(3-methylbenzyl)-cyclohexanecarboxamide;
cis-4-{[4-(dimethylamino)quinazolin-2-yl]amino}-N-(2-methylbenzyl)-cyclohexanecarboxamide;
cis-4-{[4-(dimethylamino)quinazolin-2-yl]amino}-N-[2-(trifluorometioxy)benzyl]-cyclohexanecarboxamide;
cis-4-{[4-(dimethylamino)quinazolin-2-yl]amino}-N-(2,3,4-trifluorobenzyl)-cyclohexanecarboxamide;
cis-4-{[4-(dimethylamino)quinazolin-2-yl]amino}-N-(2,4,5-trifluorobenzyl)-cyclohexanecarboxamide;
cis-4-{[4-(dimethylamino)quinazolin-2-yl]amino}-N-(3,4,5-tirifluorobenzyl)-cyclohexanecarboxamide;
cis-4-{[4-(dimethylamino)quinazolin-2-yl]amino}-N-(2,3,6-trifluorobenzyl)-cyclohexanecarboxamide;
cis-4-{[4-(dimethylamino)quinazolin-2-yl]amino}-N-[3-fluoro-5-(trifluoromethyl)benzyl]-cyclohexanecarboxamide;
cis-4-{[4-(dimethylamino)quinazolin-2-yl]amino}-N-[4-fluoro-2-(trifluoromethyl)benzyl]-cyclohexanecarboxamide;
cis-4-{[4-(dimethylamino)quinazolin-2-yl]amino}-N-[2-fluoro-4-(trifluoromethyl)benzyl]-cyclohexanecarboxamide;
cis-4-{[4-(dimethylamino)quinazolin-2-yl]amino}-N-[4-fluoro-3-(trifluoromethyl)benzyl]-cyclohexanecarboxamide;
cis-4-{[4-(dimethylamino)quinazolin-2-yl]amino}-N-[2-fluoro-3-(trifluoromethyl)benzyl]-cyclohexanecarboxamide;
cis-N-[4-chloro-3-(trifluoromethyl)benzyl]-4-{[4-(dimethylamino)quinazolin-2-yl]amino}-cyclohexanecarboamide;
cis-N-(2-chloro-6-fluorobenzyl)-4-{[4-(dimethylamino)quinazolinl-2-yl]amino}-cyclohexanecarboxamide;
cis-N-(3-chloro-4-fluorobenzyl)-4-{[4-(dimethylamino)quinazolin-2-yl]amino}-cyclohexanecarboxamide;
cis-N-(2-chloro-4-fluorobenzyl)-4-{[4-(dimethylamino)quinazolin-2-yl]amino -cyclohexanecarboxamide;
cis-N-[2-chloro-5-(trifluoromethyl)benzyl]-4-{[4-(dimethylamino)quinazolin-2-yl]amino}-cyclohexanecarboxamide;
cis-N-[2-(difluoromethoxy)benzyl]-4-{[4-(dimethylamino)quinazolin-2-yl]amino}-cyclohexanecarboxamide;
cis-N-[3-(difluoromethoxy)benzyl]-4-{[4-(dimethylamino)quinazolin-2-yl]amino}-cyclohexanecarboxamide;
cis-4-{[4-(dimethylamino)quinazolin-2-yl]amino}-N-[3-(trifluoromethoxy)benzyl]-cyclohexanecarboxamide;
cis-N-(2,6-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-[(1R)-1-phenylethyl]-cyclohexanecarboxamide;
cis-4-{[4-(dimethylamino)quinazolin-2-yl]amino}-N-[(1S)-1-(4-methoxyphenyl)ethyl)-cyclohexanecarboxamide;
cis-1-[bis(4-methoxyphenyl)methyl]-4-{[4-(dimethylamino)quinazolin-2-yl]amino}-cyclohexanecarboxamide;
cis-4-{[4-(dimethylamino)quinazolin-2-yl]amino}-N-[2-(trifluoromethyl)benzyl]-cyclohexanecarboxamide;
cis-4-{[4-(dimethylamino)quinazolin-1-yl]amino}-N-9H-fluoren-9-ylcyclohexanecarboxamide;
cis-4-{[4-(dimethylamino)quinazolin-2-yl]amino}-N-[4-(methylsulfonyl)benzyl]-cyclohexanecarboxamide; and
cis-N-(6-chloropyridin-3-yl)-4-{[4-(dimethylamino)quinazolin-2-yl]amino}- cyclohexanecarboxamide;
   or a pharmaceutically acceptable salt, hydrate or solvate thereof.

In some embodiments of the present invention, R₁ is selected from the group consisting of:
(i) C₁₋₅ alkyl, and
C₁₋₅ alkyl substituted by substituent(s) independently selected from the group consisting of:
   carbocyclic aryl,
   carbocyclic aryl substituted by substituent(s) independently selected from the group consisting of:
      C₁₋₅ alkoxy, and
      C₁₋₅ alkoxy substituted by halogen,
(ii) carbocyclic aryl, and carbocyclic aryl substituted by substituent(s) independently selected from the group consisting of:
   halogen, and
   C₁₋₇ alkoxy,
R₄ is —N(R₄ₐ)(R_{4b}) wherein R₄ₐ and R_{4b} are each independently C₁₋₅ alkyl;
L is Formula (XIII) wherein R₅ is hydrogen; A is a single bond and B is a single bond or —CH₂—; and
Y is —C(O)O— or —OC(O)—;
   wherein carbocyclic aryl is phenyl or naphityl; and
   halogen is fluoro, chloro, bromo, or iodo;
or a phannaceutically acceptable salt, hydrate or solvate thereof.

In some embodiments of the present invention, R₄ is —N(CH₃)₂; or a pharmaceutically acceptable salt, hydrate or solvate thereof.

In some embodiments of the present invention, R₁ is selected from the group consisting of:
carbocyclic aryl, and
carbocyclic aryl substituted by substituent(s) independently selected from the group consisting of:
   halogen,
   C₁₋₁₀ alkyl,
C₁₋₁₀ alkyl substituted by halogen,
   C₁₋₇ alkoxy, and
   C₁₋₇ alkoxy substituted by halogen,
R₄ is —N(R₄ₐ)(R_{4b}) wherein R₄ₐ and R_{4b} are each independently C₁₋₅ alkyl;

L is Formula (VI) or (IX) wherein A and B are each independently a single bond or —CH₂—; and
Y is —C(O)—,
   wherein carbocyclic aryl is phenyl; and
   halogen is fluoro, chloro, bromo, or iodo;
or a pharmaceutically acceptable salt, hydrate or solvate thereof.

In some embodiments of the present invention, R₄ is —N(CH₃)₂; R₅ and R₆ are both hydrogen; and A is a single bond, and B is —CH₂—; or A is a —CH₂—, and B is a single bond, or a pharmaceutically acceptable salt, hydrate or solvate thereof.

In some embodiments, compounds of the present invention are of Formula (I) wherein the compound is selected from the group consisting of:
3,4-dichloro-N-[((1R,3S)-{[4-(dimethylamino)quinazolin-2-yl]amino}cyclopentyl)-methyl]benzamide;
N-[(1S,3R)-3-({[4-(dimethylamino)quinazolin-2-yl]amino}methyl)cyclopentyl]-4-fluorobenzamide;
4-chloro-N-[((1R,3S)-3-{[4-(dimethylamino)quinazolin-2-yl]amino}cyclopentyl)methlyl]-benzamide; and
N-[((1R,3 S)-3-{[4-(dimethylamino)quinazolin-2-yl]amino}cyclopentyl)methyl]-3,5-difluorobenzamide;
   or a pharmaceutically acceptable salt. hy,drate or sol03te thereof

In some embodiments, compounds of the present invention are of Formula (I) wherein the compound is selected from the group consisting of:
N-[((1R,3S)-3-{(4-(dimethylamino)quinazolin-1-yl]amino}cyclopentyl)methyl]-3,5-dimethoxybenzamide;
2,4,6-trichloro-N-[((1R,3S)-3-{(4-(dimethylamino)quinazolin-2-yl]amino}cyclopentyl)-methyl]benzamide;
N-[((1R,3S)-3-{[4-(dimethylamino)quinazolin-2-yl]amino}cyclopentyl)methyl]-3-fluoro-4-(trifluoromethyl)benzamide;
N-[((1R,3S)-3-{[4-(dimethylamino)quinazolin-2-yl]amino}cyclopentyl)methyl]-4-(trifluoromethoxy)benzamide; and
N-[(1S,3R)-3-({[4-(dimethylamino)quinazolin-2-yl]amino}methyl)cyclopentyl]-2,4-difluorobenzamide;
   or a pharmaceutically acceptable salt, hydrate or solvate thereof.

In some embodiments of the present invention, Q is Formula (Ila) and can be represented by the following formula: 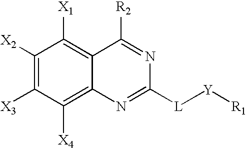
or a pharmaceutically acceptable salt, hydrate or solvate thereof, wherein X₁-X₄, R₂, L, Y, and R₁ are as described herein, supra and infra.

In some embodiments of the present invention, R₁ is selected from the group consisting of:
(i) C₁₋₈ alkyl, and C₁₋₈ alkyl substituted by carbocyclic aryl,
(ii) carbocyclic aryl, and carbocyclic aryl substitited by subttituent(s) independently selected from the group consisting of:
   halogen,
   C₁₋₁₀ alkyl,
   C₁₋₁₀ alkyl substituted by halogen,
   C₁₋₇ alkoxy, and
   C₁₋₇ alkoxy substituted by halogen,
R₂ is —N(R₂ₐ)(R_{2b}), wherein R₂ₐ and R_{2b} are each independently C₁₋₅ alkyl;
L is Formula (V) wherein R₅ and R₆ are both hydrogen; A and B are both a single bond;
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is —C(O)—;
   wherein carbocyclic aryl is phenyl; and
   halogen is fluoro, chloro, bromo, or iodo;
or a pharmaceutically acceptable salt, hydrate or solvate thereof.

In some embodiments of the present invention, R₂ is —N(CH₃)₂; and X₁, X₂, X₃ and X₄ are independently selected from the group consisting of hydrogen, fluoro, and methyl; provided that at least one substituent selected from the group consisting of X₁, X₂, X₃ and X₄ is not hydrogen; or a pharmaceutically acceptable salt, hydrate or solvate thereof.

In some embodiments, compounds of the present invention are of Formula (I) wherein the compound is selected from the group consisting of:
N-(cis-4-{[4-(dimethylamino)-6-methylquinazolin-2-yl]amino}cyclohexyl)-2,2-diphenylacetamide;
N-(cis-4-{[4-(dimethylamino)-6-methylquinazolin-2-yl]amino}cyclohexyl)-4-fluoro-3-(trifluoromethyl)benzamide;
N-(cis-4-{[4-(dimethylamino)-6-methylquinazolin-2-yl]amino}cyclohexyl)-3,5-bis(trifluoromethyl)benzamide; and
N-(cis-4-{[4-(dimethylamino)-6-methylquinazolin-2-yl]amino}cyclohexyl)-3,4,5-trimethoxybenzamide;
or a pharmaceutically acceptable salt, hydrate or solvate thereof.

In some embodiments, compounds of the present invention are of Formula (I) wherein the compound is selected from the group consisting of:
3-chloro-N-(cis-4-{[4-(dimethylamino)-6,7-difluoroquinazolin-2-yl]amino}cyclohexyl)-benzamide;
3,4-dichloro-N-(cis-4-{[4-(dimethylamino)-6,7-difluoroquinazolin-2-yl]amino}cyclohexyl)-benzamide;
N-(cis-4-{[4-(dimethylamino)-6,7-difluoroquinazolini-2-yl]amino}cyclohexyl)-3,5-dimethoxybenzamide;
N-(cis-4-{[4-(dimethylamino)-6,7-difluoroquinazolin-2-yl]amino}cyclohexyl)benzamide;
N-(cis-4-{[4-(dimethylamino)-6,7-difluoroquinazolin-2-yl]amino}cyclohexyl)-4-methylbenzamide;
N-(cis-4-{[4-(dimethylamino)-6,7-difluoroquinazolin-2-ylamino}cyclohexyl)-4-fluorobenzamide;
N-(cis-4-{[4-(dimethylamino)-6,7-difluoroquinazolin-2-yl]amino}cyclohexyl)-3-methoxybenzamide;
N-(cis4-{[4-(dimethylamino)-6,7-difluoroquinazolin-2-yl]amino}cyclohexyl)-3,4-difluorobenzamide; and
N-(cis-4-{[4-(dimetilylamino)-6,7-difluoroquinazolin-2-yl]amino}cyclohexyl)-3-trifluoromethyl)benzamide;
   or a pliamiacetitically acceptable salt, hydrate or solvate thereof.

In some embodiments of the present invention, R₁ is selected from the grouip consisting of:
(i) C₁₋₈ alkyl, and
C₁₋₈ alkyl substituted by substituent(s) independently selected from the group consisting of:
   carbocyclic aryl,
   carbocyclic aryl substituted by substituent(s) independently selected from the group consistinig of:
      halogen,
      C₁₋₅ alkyl,
      C₁₋₅ alkyl substituted by halogen,
      C₁₋₅ alkoxy, and
      C₁₋₅ alkoxy substituted by halogen,
(ii) heterocyclyl, and heterocyclyl substituted by halogen,
R₂ is —N(R₂ₐ)(R_{2b}), wherein R₂ₐ and R_{2b} are each independently C₁₋₅ alkyl;
L is Formula (XIII);
X₁, X₂, X₃ and X₄ are independently hydrogen or halogen; provided that at least one substituent selected from the group consisting of X₁, X₂, X₃ and X₄ is not hydrogen; and
Y is —C(O)NR₇— wherein R₇ is hydrogen or C₁₋₅ alkyl;
   wherein carbocyclic aryl is phenyl;
   heterocyclyl is pyridyl; and
   halogen is fluoro, chloro, bromo, or iodo;
or a pharmaceutically acceptable salt, hydrate or solvate thereof.

In some embodiments of the present invention, R₂ is —N(CH₃)₂; L is Formula (XIII) wherein A and B are both a single bond; X₁, X₂, X₃ and X₄ are independently hydrogen or fluoro; provided that at least one stibstittieit selected from the group consisting of X₁, X₂, X₃ and X₄ is not hydrogen; and Y is —C(O)NH—; or a pharmaceutically acceptable salt, hydrate or solvate thereof.

In some embodiments, compounds of the present invention are of Formula (I) wherein the compound is selected from the group consisting of:
cis-N-benzyl-4-{[4-(dimethylamino)-6,7-difluoroquinazolin-2-yl]amino}-cyclohexanecarboxamide;
cis-N-(3,5-dimethoxybenzyl)-4-{[4-(dimethylamino)-6,7-difluoroquinazolin-2-yl]amino}-cyclohexanecarboxamide;
cis-4-{[4-(dimethylamino)-6,7-difluoroquinazolin-2-yl-]amino}-N-(3-methoxybenzyl)-cyclohexanecarboxamide;
cis-N-[(6-chloropyridin-3-yl)methyl]-4-{[4-(dimethylamino)-6,7-difluoroquinazolin-2-yl]-amino}cyclohexanecarboxamide;
cis-4-{[4-(dimethylamino)-6,7-difluoroquinazolin-2-yl]amino}-N-[3-(trifluoromethyl)-benzyl]cyclohexanecarboxamide;
cis-4-{[4-(dimethylamino)-6,7-difluoroquinazolin-2-yl]amino}-N-[4-(trifluoromethyl)-benzyl]cyclohexanecarboxamide;
cis-N-[3,5-bis(trifluoromethyl)benzyl]-4-{[4-(dimethylamino)-6,7-difluoroquinazolin-2-yl]amino}cyclohexanecarboxamide;
cis-4-{[4-(dimethylamino)-6,7-difluoroquinazolin-2-yl]amino}-N-(3-iodobenzyl)-cyclohexanecarboxamide; and
cis-N-[1-(4-bromophenyl)ethyl]-4-{[4-(dimethylamino)-6,7-difluoroquinazolin-2-yl]amino}cyclohexanecarboxamide;
or a pharmaceutically acceptable salt, hydrate or solvate thereof.

In some embodiments, compounds of the present invention are of Formula (I) wherein the compound is selected from the group consisting of:
cis-4-{[4-(dimethylamino)-6,7-difluoroquinazolin-2-yl]amino}-N-(4-methylbenzyl)-cyclohexanecarboxamide;
cis-N-(3-chlorobenzyl)-4-{[4-(dimethylamino)-6,7-difluoroquinazolin-2-yl]amino}-cyclohexanecarboxamide;
cis-4-{[4-(dimethylamino)-6,7-difluoroquinazolin-2-yl]amino}-N-[(1R)-1-(3-methoxyphenyl)ethyl]cyclohexanecarboxamide;
cis-4-({4-(dimethylamino)-6,7-difluoroquinazolin-2-yl]amino}-N-(4-methoxybenzyl)-cyclohexanecarboxamide;
cis-N-(2,4-dichlorobenzyl)-4-{[4-(dimethylamino)-6,7-difluoroquinazolin-2-yl]amino}-cyclohexanecarboxamide;
cis-N-(3,5-dichlorobenzyl)-4-{[4-(dimethylamino)-6,7-difluoroquinazolin-2-yl]amino}-cyclohexanecarboxamide;
cis-N-(4-bromobenzyl)-4-{[4-(dimethylamino)-6,7-difluoroquinazolin-2-yl]amino}-cyclohexanecarboxamide;
cis-N-(2-bromobenzyl)-4-{[4-(dimethylamino)-6,7-difluoroquinazolin-2-yl]amino}-cyclohexanecarboxamide;
cis-4-{[4-(dimethylamino)-6,7-difluoroquinazolin-2-yl]amino}-N-(4-(trifluoromethoxy)-benzyl]cyclohexanecarboxamide; and
cis-4-{[4-(dimethylamino)-6,7-difluoroquinazolin-2-yl]amino}-N-[(1S)-1-(4-methylphenyl)ethyl]cyclohexanecarboxamide,

or a pharmaceutically acceptable salt, hydrate or solvate thereof.

In some embodiments of the present invention, Q is Formula (IIb) and can be represented by the following formula: 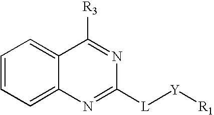
or a pharmaceutically acceptable salt, hydrate or solvate thereof, wherein R₃, L, Y, and R₁ are as described herein, supra and infra.

In some embodiments of the present invention, R₁ is selected from the group consisting of:
R₁ is selected from the group consisting of:
C₁₋₈ alkyl, and
C₁₋₈ alkyl substituted by substituent(s) independently selected from the group consisting of:
   carbocyclic aryl,
   carbocyclic aryl substituted by substituent(s) independently selected from the group consisting of:
      halogen,
      C₁₋₅ alkyl, and
      C₁₋₅ alkoxy,
R₃ is C₁₋₅ alkyl;
L is Formula (XIII); whereiii R₅ and R₆ are both hydrogen; A and B are both a single bond;
Y is —C(O)NR₇—;
   wherein carbocyclic aryl is phenyl; and
   halogen is fluoro, chloro, bromo, or iodo;
or a pharmaceutically acceptable salt, hydrate or solvate thereof.

In some embodiments of the present invention, R₃ is isopropyl; and Y is —C(O)NH—; or a pharmaceutically acceptable salt, hydrate or solvate thereof.

In some embodiments, compounds of the present invention is:
cis-N-(3-chlorobenzyl-4-[(4-isopropylquinazolin-2-yl]amino]cyclohexanecarboxamide;

or a pharmaceutically acceptable salt, hydrate or solvate thereof.

In some embodiments of the present invention, R₁ is selected from hydrogen, —CO₂tBu, or —CO₂Bn (Bn is a benzyl group);
R₂ is —N(R₂ₐ)(R_{2b}), wherein R₂ₐ is hydrogen or C₁₋₅ alkyl; R2b is C₁₋₅ alkyl;
R₃ is C₁₋₅ alkyl;
R₄ is —N(R₄ₐ)(R_{4b}) wherein R₄ₐ is hydrogen or C₁₋₅ alkyl; R_{4b} is C₁₋₅ alkyl;
L is selected from Formula (V), (VIII), (IX), (XIII), (XVI), or (XVII);
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is a single bond;
or a pharmaceutically acceptable salt, hydrate, or solvate thereof.

One aspect of the present invention pertains to pharmaceutical compositions comprising at least one compounds as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, including bulimia, anorexia, mental disorders including manic depression, schizophrenia, delirium, appetite and eating disorders, cardiovascular disease, hypertension, dyslipidemia, myocardialinfarction, binge eating disorders dementia, stress, cognitive disorders, attention deficit disorder, substance abuse disorders and dyskinesias including Parkinson's disease, epilepsy, and addiction comprising administering to an individual suffering from the condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a pharmaceutical composition thereof, for use in a method of treatment of the human or animal body by therapy.

One aspect of the present invention pertains to compounds of the present invention, as described herein or a pharmaceutical composition thereof, for use in a method of prophylaxis or treatment of an eating disorder, obesity or an obesity related disorder of the human or animal body by therapy.

One aspect of the present invention pertains to compounds of the present invention, as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder obesity or obesity related disorders.

One aspect of the present invention pertains to compounds of the present invention, as described herein, for the manufacture of a medicament for use in the prophylaxis or treatment of anxiety, depression, schizophrenia, addiction, or epilepsy.

One aspect of the present invention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces iveight gain of the individual. In some embodiments, the modulation of the MCH receptor is for prophylaxis or treatment of anxiety, depression, schizophrenia, addiction, or epilepsy.

In some embodiments, the individual is a mammal.

In some embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound, as described herein, and a pharmaceutically acceptable carrier.

One embodiment of the invention includes any compound of the invention which selectively binds an MCH receptor, such selective binding is preferably demonstrated by a Ki for one or more other GPCR(s), preferably NPY, being at least 10-fold greater than the Ki for any particular MCH receptor, preferable MCHR1.

As used herein, the term “alkyl” is intended to denote hydrocarbon compounds including straight chain and branched chain, including for example but not limited to methyl, ethyl, n-propyl, isopropyl, n-butyl, sec-butyl, tert-butyl, n-pentyl, isopentyl, tert-pentyl, n-hexyl, and the like.

The term “alkoxy” is intended to denote substituents of the formula —O-alkyl.

At various places in the present specification substituents of compounds of the invention are disclosed in groups. It is specifically intended that the invention include each and every individual subcombination of the members of such groups.

G-protein coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequelae such as stimulation of adenylyl cyclase. Melanin Concentrating Hormone (MCH), a cyclic peptide, has been identified as the endogenous ligand of the orphan G-protein coupled receptor SLC-1. See, for example. Shimomura et al., Biochem. Biophys. Res. Commun. 261, 622-26 (1999). Studies have indicated that MCH acts as a neurotransmitter/modulator/regulator to alter a number of behavioral responses.

Mammalian MCH (19 amino acids) is highly conserved between rat, mouse, and human, exhibiting 100% amino acid identity, but its physiological roles are less clear. MCH has been reported to participate in a variety of processes including feeding, water balance, energy metabolism, general arousal/attention state, memory and cognitive functions, and psychiatric disorders. For reviews, see 1. Baker, Int. Rev. Cytol. 126:1-47 (1991); 2. Baker, TEM 5:120-126 (1994); 3. Nahon, Critical Rev. in Neurobiol 221:21-262, (1994); 4. Knigge et al., Peptides 18(7):1095-1097, (1996). The role of MCH in feeding or body weight regulation is supported by Qu et al., Nature 380:243-247, (1996), demonstrating that MCH is over expressed in the hypothalamus of ob/ob mice compared with ob/+mice, and that fasting further increased MCH mRNA in both obese and normal mice during fasting. MCH also stimulated feeding in normal rats when injected into the lateral ventricles as reported by Rossi et al., Endocrinology 138:351-355, (1997). MCH also has been reported to functionally antagonize the behavioral effects of α-MSH; see: Miller et al., Peptides 14:1-10, (1993); Gonzalez et al, Peptides 17:171-177, (1996); and Sanchez et al., Peptides 18:3933-396, (1997). In addition, stress has been shown to increase POMC mRNA levels while decreasing the MCH precursor preproMCH (ppMCH) mRNA levels; Presse et al., Endocrinology 131:1241-1250, (1992). Thus MCH can serve as an integrative neuropeptide involved in the reaction to stress, as well as in the regulation of feeding and sexual activity; Baker, Int. Rev. Cytol. 126:1-47, (1991): Knigge et al., Peptides 17:1063-1073, (1996).

The localization and biological activities of MCH peptide suggest that the modulation of MCH receptor activity can be useftil in a number of therapeutic applications. MCH is expressed in the lateral hypothalamus, a brain area implicated in the regulation of thirst and hunger: Grillon et al., Neuropeptides 31:131-136, (1997); recently orexins A and B, which are potent orexigenic agents, have been shown to have very similar localization to MCH in the lateral hypothalamus; Sakurai et al., Cell 92:573-585 (1998). MCH mRNA levels in this brain region are increased in rats after 24 hours of food-deprivation; Herve and Fellmann, Neurpeptides3l :237-242 (1997); after insulin injection, a significant increase in the abundance and staining intensity of MCH immunoreactive perikarya and fibres was observed concurrent with a significant increase in the level of MCH mRNA; Bahjaoui-Bouhaddi et al., Neuropeptides 24:251-258(1994). Consistent with the ability of MCH to stimulate feeding in rats; Rossi et al., Endocrinology 138:351-355, (1997); is the observation that MCH mRNA levels are upregulated in the hypothalami of obese ob/ob mice; Qu et al., Nature 380:-743-247, (1996); and decreased in the hypothalami of rats treated with leptin, whose food intake and body weight gains are also decreased; Sahu, Endocrinology 139:795-798, (1998), MCH appears to act as a functional antagonist of the melanocortin system in its effects on food intake and on hormone secretion within the HPA (hypothalamopituitary/adrenal axis); Ludwig et al. Am. J. Physiol. Endocrinol. Metab. 274:E627-E633, (1998). Together these data suggest a role for endogenous MCH in the regulation of energy balance and response to stress, and provide a rationale for the development of specific compounds acting at MCH receptors for use in the treatment of obesity and stress-related disorders.

Accordingly, a MCH receptor antagonist is desirable for the prophylaxis or treatment of obesity or obesity related disorders. An obesity related disorder is a disorder that has been directly or indirectly associated to obesity, such as, type II diabetes, syndrome X, impaired glucose tolerance, dyslipidaemia, hypertension, coronary heart disease and other cardiovascular disorders including atherosclerosis, insulin resistance associated with obesity and psoriasis, for treating diabetic complications and other diseases such as polycystic ovarian syndrome (PCOS), certain renal diseases including diabetic nephropathy, glomerulonepliritis, glomerular sclerosis, neplirotic syndrome, hypertensive nephrosclerosis, end-stage renal diseases and microalbuminuria as Nvell as certain eating disorders.

In species studied to date, a major portion of the neurons of the MCH cell group occupies a rather constant location in those areas of the lateral hypothalamus and subthalamus where they lie and may be a part of some of the so-called “extrapyramidal” motor circuits. These involve substantial striato- and pallidofugal pathways involving the thalamus and cerebral cortex, hypothalamic areas, and reciprocal connections to subthalamic nucleus, substantia nigia, and mid-brain centers. Bittencourt et al., J. Comp. Neurol. 319:218-245, (1992). In their location, the MCH cell group may offer a bridge or mechanism for expressing hypothalamic visceral activity with appropriate and coordinated motor activity. Clinically it can be of some value to consider the involvement of this MCH system in movement disorders, such as Parkinson's disease and Huntingdon's Chorea in which extrapyramidal circuits are known to be involved.

Human genetic linkage studies have located authentic hMCH loci on chromosome 12 (12q23-24) and the variant hMCH loci on chromosome 5 (5q12-13) (Pedeutour et al., 1994). Locus 12q23-24 coincides with a locus to which autosomal dominant cerebellar ataxia type II (SCA2) has been mapped; Auburger et al., Cytogenet. Cell. Genet. 61:252-256 (1992); Twells et al., Cytogenet. Cell. Genet. 61:262-265, (1992). This disease comprises neurodegenerative disorders, including an olivopontocerebellar atrophy. Furthermore, the gene for Darier's disease, has been mapped to locus 12q23-24; Craddock et al., Hum. Mol. Genet. 2:1941-1943, (1993). Dariers' disease is characterized by abnormalities I keratinocyte adhesion and mental illnesses in some families. In view of the functional and neuroanatomical patterns of the MCH neural system in the rat and human brains, the MCH gene can represent a good candidate for SCA2 or Darier's disease. Interestingly, diseases with high social impact have been mapped to this locus. Indeed, the gene responsible for chronic or acute forms of spinal muscular atrophies has been assigned to chromosome 5q12-13 using genetic linkage analysis; Melki et al., Nature (London) 344:767-768, (1990); Westbrook et al., Cytogenet. Cell. Genet. 61:225-231, (1992). Furthermore, independent lines of evidence support the assignment of a major schizophrenia locus to chiomosome 5q11.2-13.3; Sherrington et al., Nature (London) 336:164-167, (1988); Bassett et al., Lancet 1:799-801, (1988); Gilliam et al., Genomics 5:940-944, (1989). The above studies suggest that MCH can play a role in neurodegenerative diseases and disorders of emotion.

Additional therapeutic applications for MCH-related compounds are suggested by the observed effects of MCH in other biological systems. For example, MCH can regulate reproductive functions in male and female rats. MCH transcripts and MCH peptide were found within germ cells in testes of adult rats, suggesting that MCH can participate in stem cell renewal and/or differentiation of early spemlatocytes; Hervieu et al., Biology of Reduction 54:1161-1172, (1996). MCH injected directly into the medial preoptic area (MPOA) or ventromedial nucleus (VMN) stimulated sexual activity in female rats; Gonzalez et al., Peptides 17:171-177, (1996). In ovariectomized rats primed with estradiol, MCH stimulated luteinizing hormone (LH) release while anti-MCH antiserum inhibited LH release; Gonzalez et al., Neuroendocrinology 66:254-262, (1997). The zona incerta, which contains a large population of MCH cell bodies, has previously been identified as a regulatory site for the pre-ovulatory LH surge; MacKenzie et al., Neuroendocrinology 39:289-295, (1984). MCH has been reported to influence release of pituitary hormones including ACTH and oxytocin. MCH analogues can also be useful in treating epilepsy. In the PTZ seizure model, injection of MCH prior to seizure induction prevented seizure activity in both rats and guinea pigs, suggesting that MCH-containing neurons can participate in the neural circuitry underlylng PTZ-induced seizure; Knigge and Wagner, Peptides 18:1095-1097, (1997). MCH has also been observed to affect behavioral correlates of cognitive functions. MCH treatment hastened extinction of the passive avoidance response in rats; McBride et al., Peptides 15:757-759, (1994); raising the possibility that MCH receptor antagonists can be beneficial for memory storage and/or retention. A possible role for MCH in the modulation or perception of pain is supported by the dense innervation of the periaqueductal grey (PAG) by MCH-positive fibers. Finally, MCH can participate in the regulation of fluid intake. ICV infusion of MCH in conscious sheep produced diuretic, natriuretic, and kaliuretic changes in response to increased plasma volume; Parkes, J. Neuroendocrinol. 8:57-63, (1996). Together with anatomical data reporting the presence of MCH in fluid regulatory areas of the brain, the results indicate that MCH can be an important peptide involved in the central control of fluid homeostasis in mammals.

In a recent citation MCHR1 antagonists surprisingly demonstrated their use as an anti-depressants and/or anti-anxiety agents. MCHR1 antagonists have been reported to show antidepressant and anxiolytic activities in rodent models, such as, social interaction, forced swimming test and ultrasonic vocalization. Therefore, MCHR1 antagonists could be useful to independently treat subjects with depression and/or anxiety. Also, MCHR1 antagonists could be useful to treat subjects that suffer from depression and/or anxiety and obesity.

This invention provides a method of treating an abnormality in a subject wherein the abnormality is alleviated by decreasing the activity of a mammalian MCCH1 receptor which comprises administering to the subject an amount of a compound which is a mammalian MCH1 receptor antagonist effective to treat the abnomnality. In separate embodiments, the abnormality is a regulation of a steroid or pituitary hormone disorder, an epinephrine release disorder, an anxiety disorder, genta gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neutroendocrine disorder, a cognitive disorder, a memory disorder, a sensory modulation and transmission disorder, a motor coordination disorder, a sensory integration disorder, a motor integration disorder, a dopaminergic function disorder, a sensory transmission disorder, an olfaction disorder, a sympathetic innervation disorder, an affective disorder, a stress-related disorder, a fluid-balance disorder, a seizure disorder, pain, psychotic behavior, morphine tolerance, opiate addiction or migraine.

Compositions of the invention can conveniently be administered in unit dosage form and can be prepared by any of the methods well known in the pharmaceutical art, for example, as described in Remington's Pharmaceutical Sciences (Mack Pub. Co., Easton, Pa., 1980).

The compounds of the invention can be employed as the sole active agent in a pharmaceutical or can be used in combination with other active ingredients which could facilitate the therapeutic effect of the compound.

Compounds of the present invention or a solvate or physiologically functional derivative thereof can be used as active ingredients in pharmaceutical compositions, specifically as a MCH receptor antagonists. By the tenn “active ingredient” is defined in the context of a “pharmaceutical composition” and shall mean a component of a pharmaceutical composition tl)at provides the primary phanmaceutical benefit, as opposed to an “inactive ingredient” which would generally be recognized as providing no pharmaceutical benefit. The term “pharmaceutical composition” shall mean a composition comprising at one active ingredient and at least one ingredient that is not an active ingredient (for example and not limitation, a filler, dye, or a mechanism for slow release), whereby the composition is amenable to use for a specified, efficacious outcome in a mammal (for example, and not limitation, a human).

Pharmaceutical compositions, including, but not limited to, pharmaceutical compositions, comprising at least one compound of the present invention and/or an acceptable salt cor solvate thereof (e.g, a pharmaceutically acceptable salt or solvate) as an active ingredient combined with at least one carrier or excipient (e.g., pharmaceutical carrier or excipient) can be used in the treatment of clinical conditions for which a MCH receptor antagonist is indicated. At least one compound of the present invention can be combined with the carrier in either solid or liquid form in a unit dose formulation. The pharmaceutical carrier must be compatible with the other ingredients in the composition and must be tolerated by the individual recipient. Other physiologically active ingredients can be incorporated into the pharmaceutical composition of the invention if desired, and if such ingredients are compatible with the other ingredients in the composition. Formulations can be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives such as suspending or emulsifying agents, non-aqueous vehicles (including edible oils), preservatives, and flavorings and colorants can be added to the liquid preparations. Parenteral dosage forms can be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

It is noted that when the MCH receptor antagonists are utilized as active ingredients in a pharmaceutical composition, these are not intended for use only in humans, but in other non-human mammals as well. Indeed, recent advances in the area of animal health-care mandate that consideration be given for the use of MCH receptor antagonists for the treatment of obesity in domestic animals (e.g., cats and dogs), and MCH receptor antagonists in other domestic animals where no disease or disorder is evident (e.g., food-oriented animals such as cows, cihickens, fish, etc.). Those of ordinary skill in the art are readily credited with understanding the utility of such compounds in such settings.

Pharmaceutically acceptable salts of the compounds of the invention can be prepared by reacting the free acid or base forms of these compounds with the appropriate base or acid in water, in an organic solvent, or in a mixture of the two; generally, nonaqueous media like ether, ethyl acetate, ethanol, isopropanol, dioxane, or acetonitrile are preferred. For instance, when the compound (I) possesses an acidic functional group, it can form an inorganic salt such as an alkali metal salt (e.g., sodium salt, potassium salt, etc.), an alkaline earth metal salt (e.g. calcium salt, magnesium salt, barium salt, etc.). and an ammonium salt. When the compound (I) possesses a basic functional group, it can form an inorganic salt (e.g., hydrochloride, sulfate, phosphate, hydrobromate, etc.) or an organic salt (e.g., acetate, maleate, fumarate, succinate, methanesulfonate, p-toluenesulfonate, citrate, tartrate, etc.).

When a compound of the invention contains optical isomers, stereoisomers, regio isomers, rotational isomners, a single substance and a mixture of them are included as a compound of the invention. For example, when a chemical formula is represented as showing no stereochenmical designation(s), such as Formula IV, then all possible stereoisomer, optical isomers and mixtures thereof are considered within the scope of that formula. Accordingly, Formula V, specifically designates the cis relationship between the two amino groups on the coyclohexyl ring and therefore this formula is also fully embraced by Formula IV.

The novel substituted quinazolines of the present invention can be readily prepared according to a variety of synthetic manipulations, all of which would be familiar to one skilled in the art. Preferred methods for the preparation of compounds of the present invention include, but are not limited to, those described in Scheme 1-6.

The common intermediate (F) of the novel substituted quinazolines can be prepared as shown in Scheme 1. Commercially available 1H,3H-quinazoline-2,4-dione (A) is converted to 2,4-dihalo-quinazoline (B) by a halogenating agent with or without a base (wherein X is halogen such as chloro, bromo, or iodo). The halogenating agent includes phosphorous oxychloride (POCl₃), phosphorous oxybromide (POBr₃), or phosphorus pentachloride (PC₃). The base includes a tertiary amine (preferably N,N-diisopropylethylamine etc.) or an aromatic amine (preferably N,N-dimethylaniline, etc.). Reaction temperature ranges from about 100° C. to 200° C., preferably about 140° C. to 180° C.

The halogen of 4-position of 2,4-dihalo-quinazoline (B) is selectively substituted by a primary or secondary amine (HNR₁ₐR_{1b}, wherein R₁ₐ and R_{1b} are as defined above) with or without a base in an inert solvent to provide the corresponding 4-substitued amino adduct (C). The base includes an alkali metal carbonate (preferably sodium carbonate or potassium carbonte, etc.) an alkali metal hydroxide (preferably sodium hydroxide etc.), or a tertiary amine (preferably N,N-diisopropylethylamine, triethylaminem or N-methylmorpholine. etc.). The inert solvent includes lower alkyl alcohol solvents (preferably methanol, ethanol, 2-propanol, or butanol, etc.), ethereal solvents (preferably tetrahydrofuran or dioxane, etc.), or amide solvents (preferably N,N-dimethylformamide or 1-methyl-pyrrolidin-2-one, etc.). Reaction temperature ranges from about 0° C. to 200° C., preferably about 10° C. to 150° C.

In turn, this is substituted by the mono-protected diamine (D), wherein R₅, R₆, A, and B are as defined above and P is a protective group, with or without a base in an inert solvent to provide 2,4-disubstituted amino quinazoline (E). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C. preferably about 80° C. to 150° C. Also this reaction can be carried out under microwvave conditions.

Representative protecting groups suitable for a wide variety of synthetic transformations are disclosed in Greene and Wuts, Protective Groups in Organic Synthesis, second edition, John Wiley & Sons, New York, 1991, the disclosure of which is incorporated herein by reference in its entirety. The deprotection of the protective group leads to the common intermediate (F) of the novel substituted quinazolines.

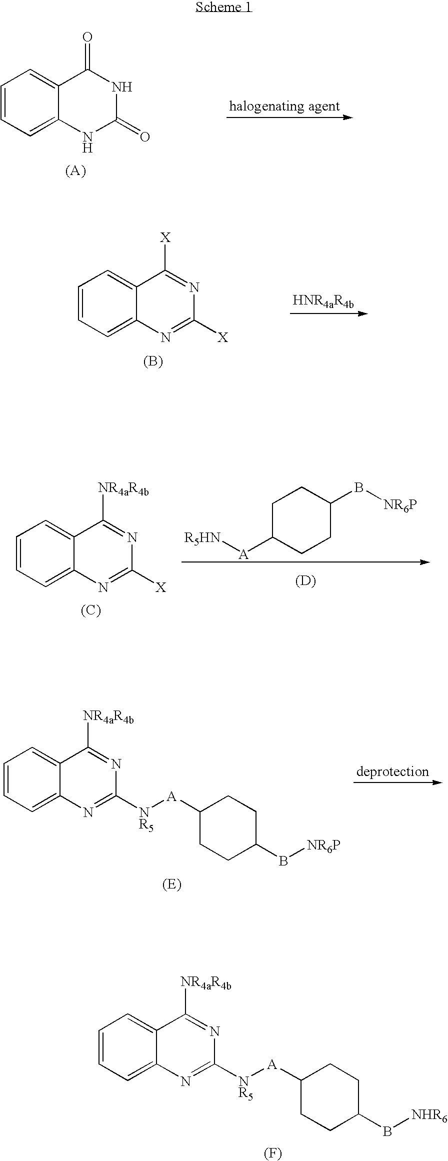

In another method, compounds of the present invention can be prepared wherein the aromatic ring is further substituted such as when Q is Formula (IIa). This method utilizes the conversion of an appropriately substituted 2-amino benzoic acid to the corresponding substinited 1H,3H-quinazoline-2,4-dione (A′); wherein X₁, X₂, X₃ and X₄ have the same meaning as described herein. Suitable conditions for the conversion to the substituted 1H,3H-quinazoline-2,4-dione (A′) are known in the art, for example, potassium cyanate, sodium cyanate, urea, and the like. In a similar method as described above in Scheme 1, the substituted 1H,3H-quinazoline-2,4-dione (A′) can be converted into useful intermediate (F′) as described generallye in Scheme 1.1

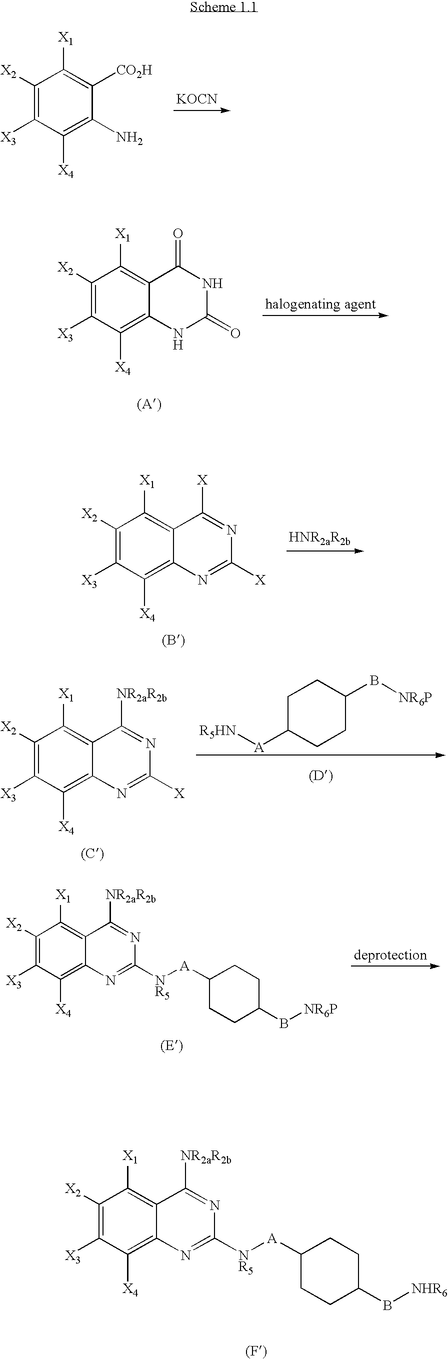

In a similar manner as described herein for intermediate (F), common intermediate (F) can be converted into novel quinazolines of Formula (I), wherein one or more of positions 5, 6, 7 or 8 on the quinazoline ring is/are substituted.

The conversion of the common intermediate (F) to the novel substituted quinazolines (G-1) of the present invention is outlined in Scheme 2.

The novel urea (G) of the present invention can be obtained by urea reaction using an isocyanate (R₁NCO) in an inert solvent with or without a base. The base includes an alkali metal carbonate (preferable sodium carbonate or potassium carbonate, etc.), an alkali metal hydrogencarbonate (preferably sodium hdrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide. etc.). Reaction temperature ranges from about −20° C. to 120° C., preferably about 0° C. to 100° C.

The amine (F) is reacted with a isothiocyanate (R₁NCS) in an inert solvent with or without a base to provide the novel thiourea (H) of the present invention. The base includes an alkali metal carbonate (preferably sodium carbonate or potassium carbonate, etc.), an alkali metal hydrogcencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chlooforn, etc.), ethereal solvents (preferably tetrahydrofuran or dioxane), aromatic solvents (preferably) benzene or toluene, etc.), or amide solvents (preferably N,N-dimethylformamide, etc.). Reaction temperature ranges from about −20° C. to 120° C., preferably about 0° C. to 100° C.

The novel urethane (I) of the present invention can be obtained by urethane reaction using R₁OCOCl, wherein X is halogen such as chloro, bromo, or iodo, in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorplioline, etc.), or an aromatic amine (preferably pyridine, imidazole, or poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferabIy about 0° C. to 100 C.

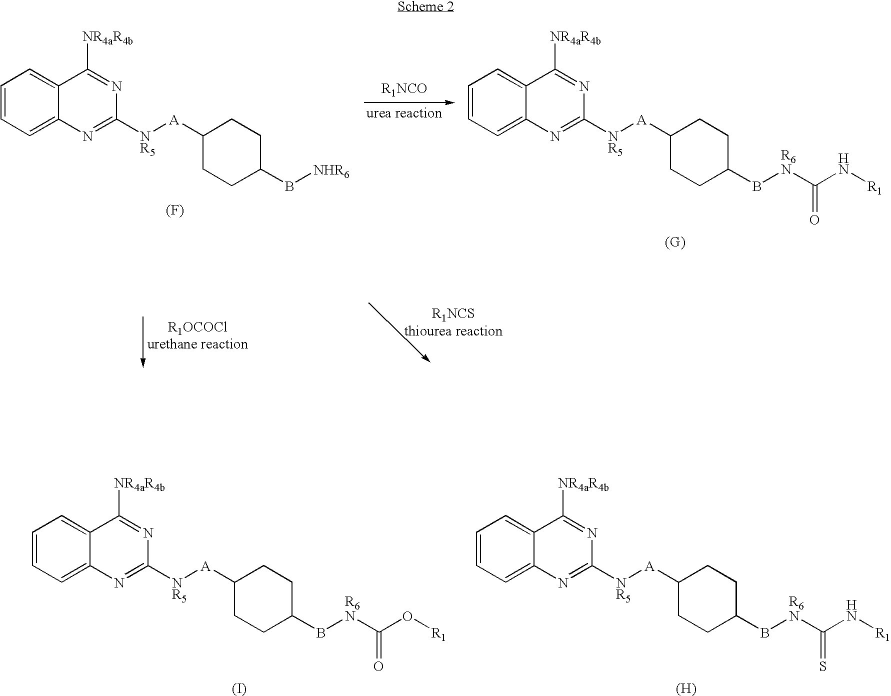

Compounds of Formula (K) can be prepared as shown in Scheme 3, [4-(Benzyloxycarbonylamino-methyl)-cyclohexyl]-carbamic acid tert-butyl ester (J) is synthesized by the method which is described in WO 01/72710. The deprotection of Boc-group is achieved by an acid to give the amine. The coupling of the amine with quinazoline core (C), which is synthesized as scheme 1, gives 2,4-disubstituted amino quinazoline. The depiotection of Z-group is achieved by hydrogen reduction to give compounds of Formula (K).

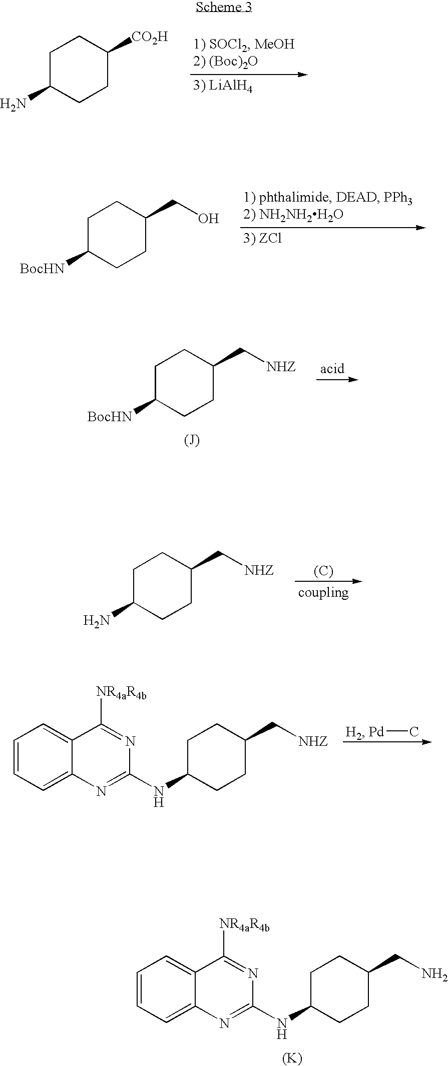

Compounds of Formula (L) can be prepared as shown in Scheme 4. The dicarboxylic acid of commercially available cis-cyclohexane-1,4-dicarboxylic acid is transformed to dibenzyl carbanate by curtius rearrangement. The deprotection of Z-group is achieved by hydrogen reduction to give the diamine. The mono-protection of the diamine can be achieved by the method described in Synthetic communications, 20, 2559-2564 (1990). The coupling of the amine with quinazoline core (C), which is synthesized as scheme 1, gives 2,4disubstituted amino quinazoline. The deprotection of Boc-group is achieved by an acid to give the amine (L).

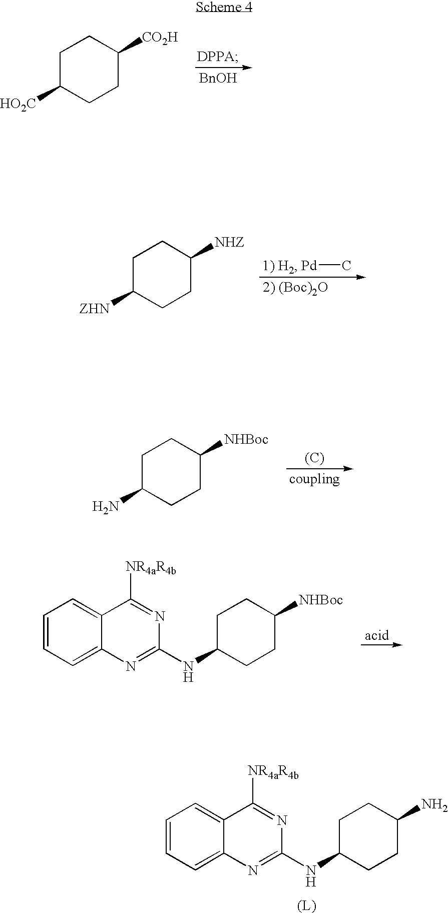

Compounds of Formula (M) can be prepared as show an in Scheme. This method utilizes 1-protected aminocyclopentane-3-carboxylic acids. The 1-protected aminocyclopentane-3-carboxylic acids that can be used are either commercially available or prepaared using methods known in the art. One particularly useful compound is (1R,3S)-N-Boc-1-aminocyclopentane-3-carboxylic acid. The 1-protected aminocyclopentane-3-carboxylic acid can be converted to the orthogonally protected 1,3-diaminocyclopentane by an arrangement, such as, the Curtius, Hoffman, Lossen, Schmidt, and the like; and subsequently protected. In the Curtius Rearrangement method, the protected amine is generated by subjecting the isocyanate intermediate with an alcohol to give a useful urethane protection group, such as, Boc, Cbz, and the like. In a subsequent step, one protecting group is removed and allowed to react in a similar manner as described herein with intermediate (C) or (C′). depicted as Q-X in Scheme 5. The second protecting group is removed to achieve amine (M).

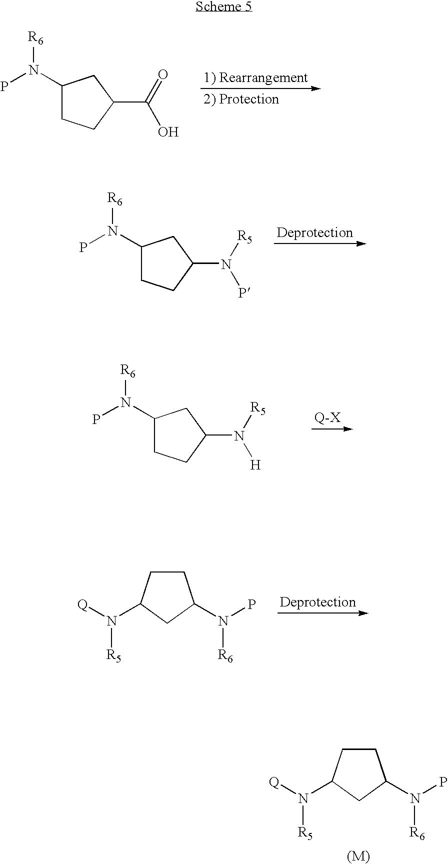

In a similar manner as described herein for intermediate (F), compound (M) can be converted into novel quinazolines of Formula (I) using methods described herein.

Novel compounds of Formula (N) of the present invention can be prepared as shown in Scheme 6. This method can utilize any of the intemnediate amines, such as amines (F), (F′), (K), (L), and (M). The amine is coupled to a 2-halopyridine carboxylic acid or similar comnpound, such as an acid halide, to give the corresponding 2-halopyridyl product. Suitable coupling methods are known in the art, such as, DCC, EDC, PyBoP, HATU, HBTU, BOP, and the like. In a subsequent step, the 2-halopyridyl product is converted to compounds of Formula (N) by treatment with an appropriate alcohol, under basic conditions such as, NaH, KH, Cs₂CO₃,K₂CO₃, Na₂CO₃ and the like. In some circumstances, a metal alkoxide can be used, such as, sodium alkoxide, potassium alkoxide and the like. The alcohol or metal alkoxide can be either substituted or unsubstituted. In a similar manner, novel compounds of Formula (O) can be prepared using a substituted or unsubstituted phenol, wherein R₈-R₁₂ represent various substitutions on the phenyl ring, including but not limited those substitutions described herein.

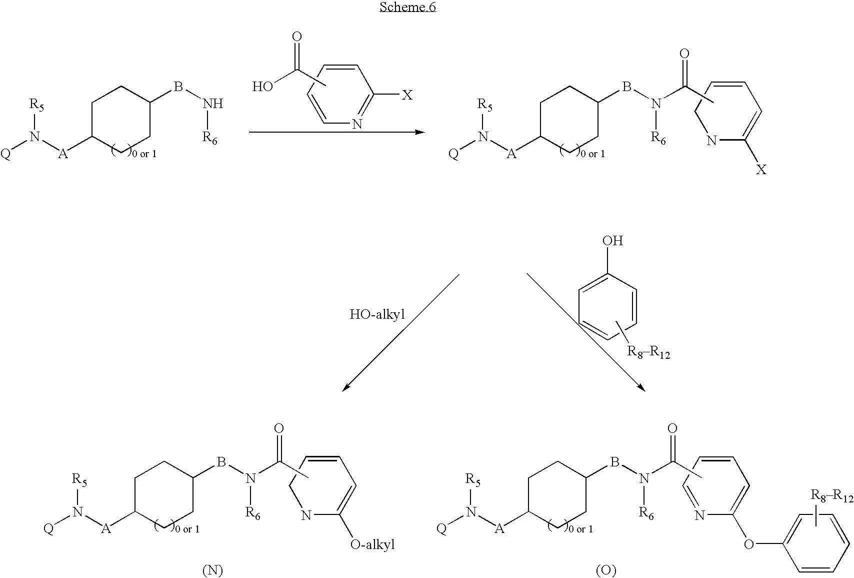

### EXAMPLES

The compounds of the inventicon and their synthesis are further illustrated by the following examples. The following examples are provided to further define the invention without, howvever, limiting the invention to the particulas of these examples. “Ambient temperature” as referred to in the folloing example is meant to indicate a temperature falling between 0° C. and 40° C. The following compounds are named by Beilstein Auto Nom Version 4.0, CS Chem Draw Ultra Version 6.0, CS compounds are named by Beilstein Auto Nom Version 4.0, CS Chem Drawv Ultra Version 6.0, CS Chein Drawv Ultra version 6.0.2, Chem Draw Ultra Version 7.0.1, or ACD Name Version 7.0.

Abbreviations used in the instant specification particularly the Schemes and Examples, are as follows:
¹H NMR: proton nuclear magnetic resonance spectrum
APCI: atmospheric pressure chemical ionization
Boc: t-butoxycarbonyl
(Boc)₂O: di-tertiary-bytyl dicarbonate
BuOH: butanol
CDCl₃ deuterated chlorofom
CH₂Cl₂: dichloromethane
CHCl₃ chloroform
Cl: chemical ionization
DIEA: diisopropylethlamine
DMA: n,N-dimethylacetamide
DMSO: dimethylsulfoxide
EI: electron ionization
ESI: electrospray ionization
Et₂O: dietihyl ether
EtOAc: acetic acid ethyl ester
EtOH: ethanol
FAB: fast atom bombardment
HATU: O(7-azabenzotriazol-1-yl)-N,N,N′,N′-tetramethyluroniumhexafluorophosphate
H₂SO₄: sulfuric acid
HCl: hydrogen chloride
K₂CO₃ : potassiunm carbonate
Me₂NH: dimethylamine
MeNH₂: methylamine
MeOH: methanol
MgSO₄: magnesium sulfate
NaH: sodium hydride
NaBH(OAc)₃: sodium triacetoxyborohydride
NaBH₃CN: sodium cyanoborohydride
NaBH₄: sodium borohydride
NaHCO₃ : sodium hydrogencarbonate
Pd/C: palladium carbon
POCl₃ : phosphoryl chloride
PVP: poly(4-vinylpyridine)
SOCl₂ : thionyl chloride
TEA: triethylamine
TFA: trifluoroacetic acid
THF: tetrahydrofuran
ZCl: benzyloxycarbonyl chloride
s: singlet
d: doublet
t: triplet
q: qualtet
dd: doublet doublet
dt: doublet triplet
ddd: doublet doublet doublet
brs: broad singlel
m: multiplet
J: coupling constant
Hz: Hertz.

### Example 1

### 1-(3,4-Dimethoxy)-phenyl)-3-[cis-4-(4-dimethylamino-quinazolin-2-ylamino)-cyclohexyl -urea hydrochloride

### Step A: Synthesis of 2,4-dichloro-quinazoline

To a suspension of 1H-quinazoline-2,4-dione (150 g, 925 mmol) in POCl₃ (549 mL, 5.89 mol) was added dimethyl-phenyl-amine (123 mL, 962 mmol). The mixture was stirred at reflux for 7 hr and concentrated. The solution was poured into ice water, and the aqueous layer was extracted with CHCl₃ (three times). The combined organic layer was dried over MgSO₄, filtrated, concentrated, and purified by flash chromatographv (silica gel, 50% CHCl₃ in hexane to 10%o EtOAc in CHCl₃) to give 2,4-dichloro-quinazoline (159 g, 86%) as a pale yellow solid.

Cl MS m/e 199 M⁺; ¹H NMR (300 MHz, CDCl₃) δ 7.71-7.81 (m, 1 H), 7.95-8.04 (m, 2 H), 8.27 (dt, J=8.3, 1.1 Hz, 1 H).

### Step B: Synthesis of (2-chloro-quinazolin-4-yl)-dimethyl-amine

A solution of 2,4-dichloro-quinazoline (102 g, 530 mmol) in THF (1.2 L) was cooled to 4 ° C. and 50% aqueous Me₂NH (139 mL, 1.33 mol) was added. The mixture was stirred at ambient temperature for 80 min. The solution was alkalized with saturated aqueous NaHCO₃ (pH =9), and the aqueous layer was extracted with CHCl₃ (three times). The combined organic layer was dried over MgSO₄, filtrated, and concentrated. The residue was suspended in 50% Et₂O in hexane (250 mL) and the mixture was stirred at ambient temperature for 30 min. The precipitate was collected by filtration, washed with 50% Et₂O in hexane, and dried at 80° C. to give (2-chloro-quinazolin-4-yl)-dimethylamine (104 g 94%) as a pale yellow solid.

ESI MS m/e 207, M⁺; ¹H NMR (300 MHz, CDCl₃) δ 3.41 (s, 6 H), 7.68 (ddd, J=8.4, 6.9, 1.4 Hz, 1 H), 7.73-7.78 ( m, 2 H), 8.00 (d, J=8.4 Hz, 1 H).

### Step C: Synthesis of (cis-4-benzyloxycarbonylamino-cyclohexyl)-carbamic acid benzyl ester

To a suspension of cis-N-cyclohexane-1,4-dicarboxylic acid (25.0 g, 145 mmol) in benzene (125 mL) were added phosphorazidic acid diphenyl ester (81.9 g, 298 mmol) and triethylamine (30.1 g, 297 mml). The reaction mixture was stirred at reflux for 2.5 hr. Benzylalcohol (32.2 g, 298 mmol) was added and the mixture was stirred at reflux for 2-4 hr. The reaction mixture was concentrated and the residue was dissolved in EtOAc and H₂O. The organic layer was separated and the aqueous layer was extracted with EtOAc (twice). The combined organic layer was washed with 1M aqueous KHSO₄, saturated aqueous NaHCO₃, and brine. The organic layer as dried over MgSO₄, filtrated, concentrated, and purified by flash chromatography (silica gel, 33% EtOAc in hexane) to give (cis-4-benzylox)carbonylamino-cyclohexyl)-carbanic acid benzyl ester (52.0 g, 94%) as a colorless oil.

ESI MS m/e 405, M+Na⁺; ¹H NMR (300 MHz, CDCl₃) δ 1.45-1.60 (m, 4 H), 1.60-1.80 (m, 4 H), 3.52-3.80 (m, 2 H). 4.70-5.00 (m, H), 5.07 (s, 4 H), 715-7.40 (m, 10 H)

### Step D: Sy nthesis of (cis-4-amino-cyclohex)yl)-carbamic acid fert-butyl ester

To a solution of (cis-4-benzyloxxcarbonylamino-cyclohexyl)-carbaiic acid benzyl ester (91.7 g, 240 mmol) in MeOH (460 mL) was added 5% Pd/C (9.17 g). The reaction mixture was stirred at ambient temperature under hydrogen atmosphere for 2.5 days filtrated through a pad of celite, and concentrated to give a diamine as a colorless oil. To a solution of the diamine in MeOH (550 mL) was added a solution of (Boc)₂O (6.59 g, 30.2 mmol) in MeOH (80 mL) dropwvise over 4 hr. The reaction mixture was stirred at ambient temperature for 1.5 days and concentrated. After dissolution with H₂O, the aqueous layer was extracted wvith CHCl₃ (three times). The combined organic layer was dried over MgSO₄, filtrated, and concentrated to give cis-(4-amino-cyclohexyxl)-carbainic acid tert-butyl ester (7.73 g, 15%, crude) as a colorless oil. The aqueous layer was concentrated and the residue was dissolved in MeOH. The solution was dried over MgSO₄, filtered, and concentrated to give a recovered diamine (32.0 g) as a colorless oil. To a solution of the recovered diamine (32.9 g, 299 mmol) in MeOH (660 mL) was added a solution of (Boc)₂O (6.20 g, 28.8 mmol) in MeOH (80 mL) dropwise over 5 hr. The reaction mixture was stirred at ambient temperature for 9.5 hr and concentrated. After dissolution with H₂O, the aqueous layer was extracted with CHCl₃ (three times). The combined organic layer was dried over MgSO₄, filtrated, and concentrated to give (cis-4-amino-cyclohexyl)-carbamiic acid tert-butyl ester (8.16 g, 16% crude) as a colorless oil. The aqueous layer was concentrated and the residue was dissolved in MeOH. The solution was dried over MgSO₄ filtrated, and concentrated to give a recovered diamine (23.1 g) as a colorless oil. To a solution of the recovered diamine (23.1 g, 202 mmol) in MeOH (462 mL) as added a solution of (Boc)₂O (4.42 g, 20.3 mmol) in MeOH (56 mL) dropwise over 4 hr. The reaction mixture was stirred at ambient temperature for 3.5 days and concentrated. After dissolution with H₂O, the aqueous layer was extracted with CHCl₃ (three times). The combined organic layer was dried over MgSO₄, filtrated, and concentrated to give (cis-4-amino-cyclohexyl)-carbamic acid tert-butyl ester (5.01 g, 10% based on starting material) as a colorless oil. The aqueous layer was concentrated and the residue was dissolved in MeOH. Thle solution was dried over MgSO₄, filtrated, and concentrated to give a recovered diamine (16.0 g) as a colorless oil. To a solution of the recovered diamnine (16.0 g, 140 mmol) in MeOH (3-20 mL) was added a solution of (Boc)₂O (3.06 g, 14.0 mmol) in MeOH (40 mL) diropise over 4 hr. The reaction mixture was stirred at ambient temperature for 17 hr and concentrated. After dissolution with H₂O, the aqueous layer was extracted vith CHCl₃ (three times). The combined organic layer was dried over MgSO₄, filtrated, and concentrated to give (cis-4-amino-cyclohexyl)-carbamic acid tert-butyl ester (3.53 g, 7% based on the starting material) as a colorless oil. The aqueous layer was concentrated and the residue was dissolved in MeOH. The solution was dried over MgSO₄, filtrated, and concentrated to give a recovered diamine (11.1 g) as a colorless oil.

### ESI NIS m/e 215, M+H⁺; ¹H NMR (300 MHz. CDCl₃) δ 1.20-1.80 (m, 8 H), 1.44 (s, 9 H), 2.78-2.95 (m, 1 H, 3.50-3.80 (m, 1 H)₅ 4.30-4.82 (m, 1 H)

### Step E: Synthesis of N²-(cis-4-amino-cyclohexyl)-N⁴,N⁴-dimethyl-quinazoline-2,4-diamine

A mixture of (2-chloro-quinazolin-4-yl)-dimethyl-amine (3.00 g, 14.4 mmol) and (cis-4-amino-cyclohexyl)-carbamic acid tert-butyl ester (3.72 g, 17.4 mmol) in 2-propanol (10 mL) was stirred at reflux for 5.5 days, poured into saturated aqueous NaHCO₃, and the aqueous layer was extracted vith CHCl₃ (three times). The combined organic layer was dried over MgSO₄, filtrated, concentrated, and purified by flash chromatography (NH-silica, 20% EtOAc in hexane) to give [cis-4-(4-dimnethylamino-quinazolin-)ylamino)-cyclohexyl]-carbamic acid tert-butyl ester including sovent (5.44 g) as a colorless oil. To a solution of the above material (5.44 g) in EtOAc (10 mL) was added 4 M hydrocen clhloride in EtOAc (50 mL). The reaction mixture was stirred at ambient temperature for 2 hr and concentrated. The residue was alkalized with saturated aqueous NaHCO₃, and the precipitate was collected by filtration to give N²-(cis-4-amino-cyclohexyl)-N⁴,N⁴-dimethyl-quinazoline-2,4-diamine (2.26 g, 55%) as a white solid. The aqueous layer was extracted CHCl, (three times). The combined organic layer was dried over MgSO₄, filtrated, and concentrated to give N²-(cis-4-amino-cyclohexyl)-N⁴,N⁴-dimethyl-quinazoline-2,4-diamine (687 mg, 17%) as a white solid.

ESI MS m/e 285, M⁺; ¹H NMR (300 MHz, DMSO-d₆) δ 1.22-1.82 (m, 8 H), 3.20 (s, 6 H). 3.38-3.52 (m, 1 H), 3.83-4.06 (m, 1 H), 6.56 (d, J=7.5 Hz, 1 H), 7.01 (t, J=7.6 Hz. 1 H), 7.29 (d, J=8.3 Hz, 1 H), 7.47 (t, J=8.3 Hz, 1 H), 7.86 (d, J=7.5 Hz, 1 H).

### Step F: Synthesis of 1-(3,4-dimethoxy)-phenyl)-3-[cis-4-(4-dimelhylamino-quinazolin-2-ylamino)-cyclohexy]-urea hydrochloride

To a solution of N²-(cis-4-amino-cyclohexyl)-N⁴,N⁴-dimethyl-quinazoline-2,4-diamine (500 mg, 1.75 mmol) in DMSO (5 mL) was added 4-isocyanato-1,2-dimethoxy-benzene (345 mg, 1.93 mmol). The mixture was stinred at ambient temperature for 1 hr and poured into water. The precipitate was filtrated, washed with water, and purified by medium-pressure liquid chromatograph (silica gel, 5% EtOAc in hexane) and flash chromatograph) (NH-silica, EtOAc) to give a pale yellow oil. To a solution of the above material in EtOAc (2 mL) was added 4 M hydrogen chloride in EtOAc (10 mL). The mixture was stirred at ambient temperature for 1 hr and concentrated. A suspension of the residue in Et₂O (10 mnL) was stirred at ambient tempareture for 1 hr. The precipitate was collected ny filtration, washed with Et₂O, and dried at 80° C. under reduced pressure to give 1-(3,4-dimethoxy-phenyl)-3-[cis-4-(4-dimethylamino-quinazolin-2-ylamino)-cyclohexyl]-urea hydrochloride (757 mg, 86% as a white solid.

ESI MS m/e 487, M (free)+Na⁺; ¹H NMR (300 MHz, CDCl₃) δ 1.68-2.07 (m, 8 H). 3.49 (s, 6 H), 3.79 (s, 6 H), 3.95, (brs, 1 H), 4.09 (brs, 1 H), 6.66 (d, J=8.7 Hz, 1 H), 6.82 (d, J=9.0 Hz, 1 H), 7.17-7.33 (m 2 H), 7.48-7.66 (m, 2 H), 7.S7 (d, J=7.3. Hz. 1 H), 8.37 (brs, 1 H), 12.77 (brs, 1 H).

### Example 2

### 1-(2,3-Dichloro-phenyl)-3-[cis-4-(4-dimethylamino-quinazolin-2-ylamino)-cyclohexylmethyl]-urea hydrochloride

### Step A: Synthesis of (cis-4-hydroxymethyl-cyclohexyl)-carbamic acid tert-butyl ester

A suspension of cis-4-amino-cyclohexanecarboxylic acid (244 g, 1.71 mol) in MeOH (2.45 L) was cooled to −8° C. Thionyl chloride (440 mL, 6.03 mol) was added dropwise. The resulting solution was stirred at ambient temperature for 4.5 hr and concentrated to give a white solid. To a suspension of the above solid in CHCl₃ (3.00 L) were added triethylamine (261 mL, 1.88 mol) and (Boc)₂O (409 g, 1.88 mol) successively. The reaction mixture was stirred at ambient temperature for 5 hr and poured into water. The aqueous layer was extracted with CHCl₃ (three times). The combined organic layer was dried over MgSO, filtrated, concentrated, and purified by flash chromatography (silica gel, 11% EtOAc in hexane to 10% MeOH in CHCl₃) and flash chromatography (NH-silica, 33% EtOAc in hexane to 9% MeOH in CHCl₃) to give a colorless oil (531 g). To a suspension cooled at −4 ° C. of lithium aluminum hydride (78.3 g, 2.06 mol) in Et₂O (7.9 L) was added a solution of the above oil (530.9 g) in Et₂O (5.3 L) below 0° C. The resulting suspension was stirred at ambient temperature for 2 hr. The reaction mixture was cooled on an ice-bath, quenched with cold water, and filtrated through a pad of celite. The filtrate was dried over MgSO₄, filtrated, and concentrated. The residue was suspended in hexane (300 mL), filtrated, washed with hexane, and dried at 70° C. to give (cis-4-hydroxymethyl-cyclohexyl)-carbamic acid tert-butyl ester (301 g, 77% as a white solid.

ESI MS m/e 252, M+Na⁺; ¹H NMR (300 MHz, CDCl₃) δ 1.61-1.36 (m,2 H), 1.45 (s, 9 H), 1.52-1.77 (m, 7 H), 3.51 (d, J=6.2 Hz, 2 H), 3.75 (brs, 1 H), 4.30-4.82 (m, 1 H).

### Step B: Synthesis of [cis-4-(benzyloxycarbonylamino-methyl)-cyclohexyl]-carbamic acid tert-butyl ester

To a solution of (cis-4-hydroxymethyl-cyclolexyl)-carbamic acid tert-butyl ester (17.7 g, 77.2 mmol) in THF (245 mL) were added triphenylphosphine (20.2 g, 77.0 mmol) and phthalimide (11.4 g, 77.5 mmol) successively. The resulting suspension was cooled on in ice-bath and 40% diethylazodicarboxylate in toluene (33.6 mL, 74.1 mmol) as added over 1 hr. The reaction mixture was stirred at ambient temperature for 2.5 days, concentrated, and purified by flash chromatography (silica gel, 33% EtOAc in hexane) to give a white solid. To a suspension of the above solid (27.5 g) in EtOH (275 mL) was added hydrazine hydrate (5.76 g, 115 mmol). The mixture was stirred at reflux for 2.25 hr, cooled, and concentrated. The residue was dissolved in 10% aqueous NaOH (350 mL) and the aqueous layer was extracted with CHCl₃ (three times). The combined organic layer was dried over MgSO₄, filtrated, and concentrated. To a solution of the above residue in CHCl₃ (275 mL) was added triethylamine (8.54 g, 84.4 mmol). The resulting solution was cooled to 0° C. and ZCl (14.4 g, 84.4 mmol) was added below 5° C. The reaction mixture was stirred at ambient temperature for 16 hr and poured into saturated aqueous NaHCO₃. The aqueous layer was extracted with CHCl₃ (three times). The combined organic layer was dried over MgSO₄, filtrated, concentrated, and purified by flash chromatography (silica gel, 2% MeOH in CHCl₃) to give [cis-4-(benzyloxycarbonylaminomethyl)-cyclohexyl]-carbamic acid tert-butyl ester (25.3 g, 91%) as a colorless oil.

ESI NIS m/e 385, M+Na⁺; ¹H NMR (300 MHz, CDCl₃), δ 1.13-1.31 (m, 2-H), 1.44 (s, 9 H), 1.48-1.75 (m, 7 H). 3.10 (t, J=6.4 Hz, 2 H), 3.72 (brs, 1 H), 4.42-4.76 (m, 1 H), 4.76-4.92 (m, 1 H), 5.09 (s, 2 H), 7.27-7.38 (m, 5 H).

### Step C: Synthesis of (cis-4-amino-cyclohexylmethyl)-carbamic acid benzyl ester

To a solution of [cis-4-(benzyloxycarbonylamino-methyl)-cyclohexyl]-carbamic acid tert-butyl ester (12.9 g, 35.6 mmol) in EtOAc (129 mL) was added 4 M hydrogen chloride in EtOAc (129 mL). The reaction mixture was stirred at ambient temperature for 3 hr, filtrated, washed with EtOAc, and dried under reduced pressure. The above solid was dissolved in saturated aqueous NaHCO₂ (pH=9). The aqueous layer was extracted with CHCl₃ (five times). The combined organic layer was dried over MgSO₄, filtrated, concentrated, and dried under reduced pressure to give (cis-4-aminocyclohexylmethyl)-carbamic acid benznyl ester (8.88 g, 95%) as a colorless oil.

ESI MS m/e 263, M+H⁺; ¹H NMR (300 MHz, CDCl₃) δ 1.36-1.98 (m, 9 H), 2.96-3.32 (m, 3 H), 5.12 (brs, 3 H), 7.36 (s. 5 H).

### Step D: Synthesis of [cis-4-(4-dimethylamino-quinazolin-2-ylamino)-cyclohexylmethyl]-carbamic acid benezylester

A mixture of (2-chloro-quinazolin-4-yl)-dimethyl-amine obtained in step B of example 1 (50 g, 258 mmol) and (cis-4-amino-cyclohexlmethyl)-carbamic acid benzy!l ester (81 g, 309 mmol) in 2-propanol (75 mL) was stirred at reflux for 7 days. The reaction mixture was poured into saturated aqueous NaHCO₃ and the aqueous layer was extracted with CHCl₃ (three times). The combined organic layer was dried over MgSO₄, filtrated, concentrated, and purified by flash chromatography (NH-silica gel, 13% to 50% EtOAc in hexane) to give [cis-4-(4-dimethylamino-quinazolin-2-ylamino)-cyclohexylmethyl)-carbamic acid benzyl ester (65.7 g, 59%) as a pale brown solid.

ESI MS m/e 434, M+H⁺; ¹H NMR (300 MHz, CDCl₃) δ 1.23-1.40 (m, 2 H), 1.52-1.73 (m, 5 H), 1.80-1.93 (m, 2 H), 3.11 (t, J=6.3 Hz, 2 H), 3.26 (s, 6 H), 4.18 -4.28 (m, 1 H), 4.82)-4.93 (m, 1 H), 4.93-5.06 (m, 1 H), 5.10 (s, 2 H), 7.01 (ddd, J=8.2, 6.5, 1.7 Hz, 1 H). 7.26-7.52 (m, 7 H), 7.81 (d, J=9.0 Hz, 1 H).

### Step E: Synthesis of N²-(cis-4-aminomethyl-cyclohexyl)-N⁴,N⁴-dimethyl-quinazoline-2,4-diamine

To a solution of [cis-4-(4-dimethylamino-quinazolin-2-ylamino)-cyclohexyl-methy-]-carbamic acid benzyl ester (12.1 g, 27.9 mmol) in MeOH (120 mL) was added 10% Pd/C (1.21 g). The mixture was stirred at 50° C. under hydrogen atmosphere for 19 hr., filtrated, concentrated, and purified by flash chromatography (NH-silica, 66% EtOAc in hexane to 15%, MeOH in CHCl₃) to give N²-(cis-4-aminomethyl-cyclohexyl)-N⁴,N⁴-dimethlyl-quinazoline-2,4-diamine (6.9 g, 83%) a pale yellow solid.

CI MS m/e 300, M+H⁺: ¹H NMR (300 MHz, CDCl₃) δ 0.90-1.51 (m, 5 H), 1.57-1.76 (m, 4 H), 1.81-1.96 (m, 2 H), 2.60 (d, J=6.4 Hz, 2 H), 3.27 (s, 6 H), 4.24-4.30 (m, 1 H), 5.04 (d, J=7.3 Hz, 1 H), 6.98 -7.04 (m, 1 H), 7.40-7.51 (m, 2 H), 7.81 (d, J=8.4 Hz, 1 H).

### Step F: Synthesis of 1-(2,3-dichloro-phenyl)-3-[cis-4-(4-dimethylamino-quinazolin-2-ylamino)cyclohexylmethyl]-urea hydrochloride

Using, the procedure for the step F of examiple 1, the title compound was obtained.

ESI MS m/e 509, M (free)+Na⁺; ¹H NMR (300 MHz, CDCl₃) δ 1.48-2.12 (m, 9 H), 3.37-3.44 (m, 2 H), 3.51 (s, 6 H), 4.3 7-4.49 (m, 1 H 6.91-7.13 (m, 3 H), 7.27 (ddd, J=8.4, 7.2, 1.2 Hz, 1 H), 7.50 (dd, J=8.6, 1.2 Hz, 1 H), 7.67 (ddd, J=8.4 7.2, 1.2 Hz, 1 H), 7.89 (d, J=8.4 Hz, 1 H), 8.17 (dd. J=8.2, 1.7Hz, 1 H), 8.24 (s, 1 H), 8.89 (d, J=8.9 Hz, 1 H), 12.42 (s, 1 H).

### Example 3

### 1-(2,6-Dichloro-phenyl)-3-[cis-4-(4-dimethylamino-quinazolin-2-)ylamino)-cyclohexylmethyl]-urea hydrochloride

### Step A: Synthesis of 1-(2,6-dichloro-phenyl)-3-[cis-4-(4-dimethylamino-quinazolin-2-ylamino)-cyclohexylmethyl]-urea hydrochloride

Using the procedure for the step F of example 1, the title compound was obtained.

ESI MS m/e 509, M (free)+Na⁺; ¹H NMR (300 MHz, CDCl₃) δ 1.51-2.06 (m, 9 H), 3.37-3.42 (m, 2 H), 3.52 (s, 6 H), 4.37-4.47 (m, 1 H), 6.35-6.45 (m, 1 H), 6.96-7.06 (m, 1 H), 7.23-7.31 (m, 3 H), 7.43-7.49 (m, 1 H), 7.61-7.68 (m, 1 H), 7.91 (d, J=7.9 Hz, 2 H), 8.72 (d, J=8.7 Hz, 1 H), 12.64 (s, 1 H).

### Example 4-845

To a solution of amines (30 O μmol) as shown below in DMSO (300 μL) were added isocyanate or isothiocyanate (60 μmol) in DNISO (200 μL) at ambient temperature. The mixture was stirred at the same temperature for 22 hr. To the reaction mixture were added 2 M MeNH, in THF (30 μL, 60 μmol) or D-gulcamine (60 μmol) in DMSO (200 μL) at ambient temperature. After stirring at the same temperature for 20 hr, the reaction mixture was filtrated through a pad of SCX, concentrated by a stream of of N₂, and purified by silica gel chromatography (silica gel, 2% to 7% 2M NH₃/MeOH in CHCl₃) and silica gel chromatography (NH-silica,20% to 50% EtOAc in hexane) to give the desired product. The product was determined by ESI-MS or APCI-MS.

### Example 846-885

To a solution of poly(4-vinylpyridine) (75 μL) in CH₂Cl₂ (200 μL) were added the amines (30 μmol) as shown below in CH₂Cl₂ (200 μL) and chloroformate (R₁OCOCl, 60 μmol) in CH₂Cl₂ (200 μL) at ambient temperature. After stirring at the same temperature for 17 hr, the reaction mixture was filtrated and concentrated by a stream of dry N₂. To the residue wvere added CH₂Cl₂ (700 μL) and PSA (300 μL). After the stirring at ambient temperature for 19 hr, the reaction mixture was filtrated and purified by silica gel chromatograph) (NH-silica, 20% EtOAc in hexane) and silica gel chromatography (silica gel, 2% to 7% 2M NH₃/MeOH in CHCl₃) to give the desired product. The product was determined by ESI-MS or APCI-MS.

Wherein the amines are selected from N²-(cis-4-amino-cyclohexyl)-N⁴,N⁴H-dimethyl-quinazoline-2,4-diamine obtained in step E of example 1 or N²-(sis-4-aminomethyl-cyclohexyl)-N⁴,N⁴-dimethyl-quinazoline-2,4-diamine obtained in step E of example 2.

| Ex. No. | compound name | MS | class |
|---|---|---|---|
| 4 | N-(3-acetylphenyl)-N′-(cis-4-{[4-(dimethylamino)quinazolin-2- | 447 (M + H) | 3 |
| | yl]amino}cyclohexyl)urea | | |
| 5 | N-1-adamantyl-N′-(cis-4-{[4-(dimethylamino)quinazolin-2- | 463 (M + H) | 3 |
| | yl]amino}cyclohexyl)urea | | |
| 6 | N-(4-acetylphenyl)-N′-(cis-4-{[4-(dimethylamino)quinazolin-2- | 447 (M + H) | 3 |
| | yl]amino}cyclohexyl)urea | | |
| 7 | N-{[(cis-4-{[4-(dimethylamino)quinazolin-2- | 433 (M + H) | 3 |
| | yl]amino}cyclohexyl)amino]carbonyl}benzamide | | |
| 8 | N-[3,5-bis(trifluoromethyl)phenyl]-N′-(cis-4-{[4- | 541 (M + H) | 3 |
| | (dimethylamino)quinazolin-2-yl]amino}cyclohexyl)urea | | |
| 9 | N-benzyl-N′-(cis-4-{[4-(dimethylamino)quinazolin-2- | 419 (M + H) | 2 |
| | yl]amino}cyclohexyl)urea | | |
| 10 | N-(2-bromophenyl)-N′-(cis-4-{[4-(dimethylamino)quinazolin-2- | 483 (M + H) | 1 |
| | yl]amino}cyclohexyl)urea | | |
| 11 | N-biphenyl-2-yl-N′-(cis-4-{[4-(dimethylamino)quinazolin-2- | 481 (M + H) | 1 |
| | yl]amino}cyclohexyl)urea | | |
| 12 | N-(4-bromophenyl)-N′-(cis-4-{[4-(dimethylamino)quinazolin-2- | 483 (M + H) | 2 |
| | yl]amino}cyclohexyl)urea | | |
| 13 | N-butyl-N′-(cis-4-{[4-(dimethylamino)quinazolin-2- | 385 (M + H) | 1 |
| | yl]amino}cyclohexyl)urea | | |
| 14 | N-(3-chlorophenyl)-N′-(cis-4-{[4-(dimethylamino)quinazolin-2- | 439 (M + H) | 3 |
| | yl]amino}cyclohexyl)urea | | |
| 15 | N-(4-chlorophenyl)-N′-(cis-4-{[4-(dimethylamino)quinazolin-2- | 439 (M + H) | 3 |
| | yl]amino}cyclohexyl)urea | | |
| 16 | N-cyclohexyl-N′-(cis-4-{[4-(dimethylamino)quinazolin-2- | 411 (M + H) | 2 |
| | yl]amino}cyclohexyl)urea | | |
| 17 | N-(3-cyanophenyl)-N′-(cis-4-{[4-(dimethylamino)quinazolin-2- | 430 (M + H) | 3 |
| | yl]amino}cyclohexyl)urea | | |
| 18 | N-(2-chlorophenyl)-N′-(cis-4-{[4-(dimethylamino)quinazolin-2- | 439 (M + H) | 1 |
| | yl]amino}cyclohexyl)urea | | |
| 19 | N-(cis-4-{[4-(dimethylamino)quinazolin-2-yl]amino}cyclohexyl)- | 433 (M + H) | 1 |
| | N′-(2,6-dimethylphenyl)urea | | |
| 20 | N-(3,4-dichlorophenyl)-N′-(cis-4-{[4-(dimethylamino)quinazolin- | 473 (M + H) | 3 |
| | 2-yl]amino}cyclohexyl)urea | | |
| 21 | N-(2,4-difluorophenyl)-N′-(cis-4-{[4-(dimethylamino)quinazolin- | 441 (M + H) | 1 |
| | 2-yl]amino}cyclohexyl)urea | | |
| 22 | N-(2,4-dichlorophenyl)-N′-(cis-4-{[4-(dimethylamino)quinazolin- | 473 (M + H) | 2 |
| | 2-yl]amino}cyclohexyl)urea | | |
| 23 | N-(3,5-dichlorophenyl)-N′-(cis-4-{[4-(dimethylamino)quinazolin- | 473 (M + H) | 3 |
| | 2-yl]amino}cyclohexyl)urea | | |
| 24 | N-(2,3-dichlorophenyl)-N′-(cis-4-{[4-(dimethylamino)quinazolin- | 473 (M + H) | 3 |
| | 2-yl]amino}cyclohexyl)urea | | |
| 25 | N-(2,6-difluorophenyl)-N′-(cis-4-{[4-(dimethylamino)quinazolin- | 441 (M + H) | 3 |
| | 2-yl]amino}cyclohexyl)urea | | |
| 26 | N-(2,5-dichlorophenyl)-N′-(cis-4-{[4-(dimethylamino)quinazolin- | 473 (M + H) | 3 |
| | 2-yl]amino}cyclohexyl)urea | | |
| 27 | N-(cis-4-{[4-(dimethylamino)quinazolin-2-yl]amino}cyclohexyl)- | 433 (M + H) | 1 |
| | N′-(2,3-dimethylphenyl)urea | | |
| 28 | ethyl N-{[(cis-4-{[4-(dimethylamino)quinazolin-2- | 415 (M + H) | 3 |
| | yl]amino}cyclohexyl)amino]carbonyl}glycinate | | |
| 29 | ethyl 3-({[(cis-4-{[4-(dimethylamino)quinazolin-2- | 477 (M + H) | 1 |
| | yl]amino}cyclohexyl)amino]carbonyl}amino)benzoate | | |
| 30 | ethyl 4-({[(cis-4-{[4-(dimethylamino)quinazolin-2- | 477 (M + H) | 2 |
| | yl]amino}cyclohexyl)amino]carbonyl}amino)benzoate | | |
| 31 | N-(cis-4-{[4-(dimethylamino)quinazolin-2-yl]amino}cyclohexyl)- | 433 (M + H) | 2 |
| | N′-(4-ethylphenyl)urea | | |
| 32 | N-(cis-4-{[4-(dimethylamino)quinazolin-2-yl]amino}cyclohexyl)- | 357 (M + H) | 3 |
| | N′-ethylurea | | |
| 33 | N-(cis-4-{[4-(dimethylamino)quinazolin-2-yl]amino}cyclohexyl)- | 447 (M + H) | 1 |
| | N′-(2-ethyl-6-methylphenyl)urea | | |
| 34 | ethyl N-{[(cis-4-{[4-(dimethylamino)quinazolin-2- | 471 (M + H) | 1 |
| | yl]amino}cyclohexyl)amino]carbonyl}lucinate | | |
| 35 | N-(cis-4-{[4-(dimethylamino)quinazolin-2-yl]amino}cyclohexyl)- | 468 (M + H) | 3 |
| | N′-(4-fluoro-3-nitrophenyl)urea | | |
| 36 | N-(cis-4-{[4-(dimethylamino)quinazolin-2-yl]amino}cyclohexyl)- | 423 (M + H) | 1 |
| | N′-(4-fluorophenyl)urea | | |
| 37 | N-(cis-4-{[4-(dimethylamino)quinazolin-2-yl]amino}cyclohexyl)- | 423 (M + H) | 3 |
| | N′-(3-fluorophenyl)urea | | |
| 38 | N-(cis-4-{[4-(dimethylamino)quinazolin-2-yl]amino}cyclohexyl)- | 423 (M + H) | 3 |
| | N′-(2-fluorophenyl)urea | | |
| 39 | N-(cis-4-{[4-(dimethylamino)quinazolin-2-yl]amino}cyclohexyl)- | 447 (M + H) | 3 |
| | N′-(4-isopropylphenyl)urea | | |
| 40 | N-(cis-4-{[4-(dimethylamino)quinazolin-2-yl]amino}cyclohexyl)- | 487 (M + H) | 1 |
| | N′-[1-(3-isopropenylphenyl)-1-methylethyl]urea | | |
| 41 | methyl N-{[(cis-4-{[4-(dimethylamino)quinazolin-2- | 475 (M + H) | 1 |
| | yl]amino}cyclohexyl)amino]carbonyl}methioninate | | |
| 42 | N-(cis-4-{[4-(dimethylamino)quinazolin-2-yl]amino}cyclohexyl)- | 371 (M + H) | 3 |
| | N′-isopropylurea | | |
| 43 | N-(cis-4-{[4-(dimethylamino)quinazolin-2-yl]amino}cyclohexyl)- | 435 (M + H) | 2 |
| | N′-(4-methoxyphenyl)urea | | |
| 44 | N-(cis-4-{[4-(dimethylamino)quinazolin-2-yl]amino}cyclohexyl)- | 464 (M + H) | 3 |
| | N′-(4-methyl-2-nitrophenyl)urea | | |
| 45 | N-(cis-4-{[4-(dimethylamino)quinazolin-2-yl]amino}cyclohexyl)- | 435 (M + H) | 2 |
| | N′-(2-methoxyphenyl)urea | | |
| 46 | N-(cis-4-{[4-(dimethylamino)quinazolin-2-yl]amino}cyclohexyl)- | 435 (M + H) | 2 |
| | N′-(3-methoxyphenyl)urea | | |
| 47 | N-(cis-4-{[4-(dimethylamino)quinazolin-2-yl]amino}cyclohexyl)- | 451 (M + H) | 1 |
| | N′-[4-(methylthio)phenyl]urea | | |
| 48 | N-(cis-4-{[4-(dimethylamino)quinazolin-2-yl]amino}cyclohexyl)- | 449 (M + H) | 2 |
| | N′-(4-methoxybenzyl)urea | | |
| 49 | N-(cis-4-{[4-(dimethylamino)quinazolin-2-yl]amino}cyclohexyl)- | 433 (M + H) | 3 |
| | N′-(3-methylbenzyl)urea | | |
| 50 | N-(cis-4-{[4-(dimethylamino)quinazolin-2-yl]amino}cyclohexyl)- | 455 (M + H) | 1 |
| | N′-1-napthylurea | | |
| 51 | N-(cis-4-{[4-(dimethylamino)quinazolin-2-yl]amino}cyclohexyl)- | 445 (M + H) | 1 |
| | N′-[(2S)-2-phenylcyclopropyl]urea | | |
| 52 | N-(cis-4-{[4-(dimethylamino)quinazolin-2-yl]amino}cyclohexyl)- | 405 (M + H) | 2 |
| | N′-phenylurea | | |
| 53 | N-(cis-4-{[4-(dimethylamino)quinazolin-2-yl]amino}cyclohexyl)- | 497 (M + H) | 1 |
| | N′-(4-phenoxyphenyl)urea | | |
| 54 | N-(cis-4-{[4-(dimethylamino)quinazolin-2-yl]amino}cyclohexyl)- | 399 (M + H) | 1 |
| | N′-pentylurea | | |
| 55 | N-(cis-4-{[4-(dimethylamino)quinazolin-2-yl]amino}cyclohexyl)- | 473 (M + H) | 1 |
| | N′-[2-(trifluoromethyl)phenyl]urea | | |
| 56 | N-(cis-4-{[4-(dimethylamino)quinazolin-2-yl]amino}cyclohexyl)- | 473 (M + H) | 3 |
| | N′-[3-(trifluoromethyl)phenyl]urea | | |
| 57 | N-(cis-4-{[4-(dimethylamino)quinazolin-2-yl]amino}cyclohexyl)- | 419 (M + H) | 2 |
| | N′-(4-methylphenyl)urea | | |
| 58 | N-(cis-4-{[4-(dimethylamino)quinazolin-2-yl]amino}cyclohexyl)- | 447 (M + H) | 1 |
| | N′-mesitylurea | | |
| 59 | N-(cis-4-{[4-(dimethylamino)quinazolin-2-yl]amino}cyclohexyl)- | 419 (M + H) | 2 |
| | N′-(3-methylphenyl)urea | | |
| 60 | N-(cis-4-{[4-(dimethylamino)quinazolin-2-yl]amino}cyclohexyl)- | 419 (M + H) | 1 |
| | N′-(2-methylphenyl)urea | | |
| 61 | N-(cis-4-{[4-(dimethylamino)quinazolin-2-yl]amino}cyclohexyl)- | 483 (M + H) | 1 |
| | N′-[1-(-napthyl)ethyl]urea | | |
| 62 | methyl N-{[(cis-4-{[4-(dimethylamino)quinazolin-2- | 491 (M + H) | 1 |
| | yl]amino}cyclohexyl)amino]carbonyl}phenylalaninate | | |
| 63 | N-(cis-4-{[4-(dimethylamino)quinazolin-2-yl]amino}cyclohexyl)- | 507 (M + H) | 1 |
| | N′-(2,4,6-trichlorophenyl)urea | | |
| 64 | N-(3-chloro-4-methylphenyl)-N′-(cis-4-{[4- | 453 (M + H) | 3 |
| | (dimethylamino)quinazolin-2-yl]amino}cyclohexyl)urea | | |
| 65 | N-(cis-4-{[4-(dimethylamino)quinazolin-2-yl]amino}cyclohexyl)- | 433 (M + H) | 1 |
| | N′-(1-phenylethyl)urea | | |
| 66 | 1-[4-(4-Dimethylamino-quinazolin-2-ylamino)-cyclohexyl]-3-(1- | 433 (M + H) | 1 |
| | phenyl-ethyl)-urea | | |
| 67 | 1-[4-(4-Dimethylamino-quinazolin-2-ylamino)-cyclohexyl]-3-(1- | 483 (M + H) | 2 |
| | napthalen-1-yl-ethyl)-urea | | |
| 68 | N-(2,6-diisopropylphenyl)-N′-(cis-4-{[4- | 489 (M + H) | 3 |
| | (dimethylamino)quinazolin-2-yl]amino}cyclohexyl)urea | | |
| 69 | N-[2-(difluoromethoxy)phenyl]-N′-(cis-4-{[4- | 471 (M + H) | 3 |
| | (dimethylamino)quinazolin-2-yl]amino}cyclohexyl)urea | | |
| 70 | metyl 2-({[4(cis-4-{[-(dimethylamino)quinazolin-2- | 463 (M + H) | 3 |
| | yl]amino}cyclohexyl)amino]carbonyl}amino)benzoate | | |
| 71 | N-(cis-4-{[4-(dimethylamino)quinazolin-2-yl]amino}cyclohexyl)- | 451 (M + H) | 2 |
| | N′-[2-(methylthio)phenyl]urea | | |
| 72 | N-(cis-4-{[4-(dimethylamino)quinazolin-2-yl]amino}cyclohexyl)- | 541 (M + H) | 1 |
| | N′-(2,3,5,6-tetrachlorophenyl)urea | | |
| 73 | N-(cis-4-{[4-(dimethylamino)quinazolin-2-yl]amino}cyclohexyl)- | 478 (M + H) | 2 |
| | N′-(2,3-dimethyl-6-nitrophenyl)urea | | |
| 74 | N-(cis-4-{[4-(dimethylamino)quinazolin-2-yl]amino}cyclohexyl)- | 507 (M + H) | 3 |
| | N′-(2,4,5-trichlorophenyl)urea | | |
| 75 | N-(cis-4-{[4-(dimethylamino)quinazolin-2-yl]amino}cyclohexyl)- | 638 (M + H) | 1 |
| | N′-(2,4,6-tribromophenyl)urea | | |
| 76 | N-(2,4-dibromo-6-fluorophenyl)-N′-(cis-4-{[4- | 579 (M + H) | 1 |
| | (dimethylamino)quinazolin-2-yl]amino}cyclohexyl)urea | | |
| 77 | N-(2,4-dibromophenyl)-N′-(cis-4-{[4-(dimethylamino)quinazolin- | 561 (M + H) | 1 |
| | 2-yl]amino}cyclohexyl)urea | | |
| 78 | N-(2,4-dichlorobenzyl)-N′-(cis-4-{[4-(dimethylamino)quinazolin- | 487 (M + H) | 1 |
| | 2-yl]amino}cyclohexyl)urea | | |
| 79 | N-(2,4-dimethoxyphenyl)-N′-(cis-4-{[4- | 465 (M + H) | 1 |
| | (dimethylamino)quinazolin-2-yl]amino}cyclohexyl)urea | | |
| 80 | N-(cis-4-{[4-(dimethylamino)quinazolin-2-yl]amino}cyclohexyl)- | 433 (M + H) | 3 |
| | N′-(2,4-dimethylphenyl)urea | | |
| 81 | N-(2,5-dimethoxyphenyl)-N′-(cis-4-{[4- | 465 (M + H) | 2 |
| | (dimethylamino)quinazolin-2-yl]amino}cyclohexyl)urea | | |
| 82 | N-(cis-4-{[4-(dimethylamino)quinazolin-2-yl]amino}cyclohexyl)- | 433 (M + H) | 3 |
| | N′-(2,5-dimethylphenyl)urea | | |
| 83 | N-(2,6-dibromo-4-fluorophenyl)-N′-(cis-4-{[4- | 579 (M + H) | 3 |
| | (dimethylamino)quinazolin-2-yl]amino}cyclohexyl)urea | | |
| 84 | N-(2,6-dichlorophenyl)-N′-(cis-4-{[4-(dimethylamino)quinazolin- | 473 (M + H) | 3 |
| | 2-yl]amino}cyclohexyl)urea | | |
| 85 | N-(2,6-diethylphenyl)-N′-(cis-4-{[4-(dimethylamino)quinazolin- | 461 (M + H) | 1 |
| | 2-yl]amino}cyclohexyl)urea | | |
| 86 | N-(2-benzylphenyl)-N′-(cis-4-{[4-(dimethylamino)quinazolin-2- | 495 (M + H) | 3 |
| | yl]amino}cyclohexyl)urea | | |
| 87 | N-(2-chloro-5-methylphenyl)-N′-(cis-4-{[4- | 453 (M + H) | 3 |
| | (dimethylamino)quinazolin-2-yl]amino}cyclohexyl)urea | | |
| 88 | N-(2-chloro-5-nitrophenyl)-N′-(cis-4-{[4- | 484 (M + H) | 2 |
| | (dimethylamino)quinazolin-2-yl]amino}cyclohexyl)urea | | |
| 89 | N-[2-chloro-6-(trifluoromethyl)phenyl]-N′-(cis-4-{[4- | 507 (M + H) | 1 |
| | (dimethylamino)quinazolin-2-yl]amino}cyclohexyl)urea | | |
| 90 | N-(2-chloro-6-methylphenyl)-N′-(cis-4-{[4- | 453 (M + H) | 1 |
| | (dimethylamino)quinazolin-2-yl]amino}cyclohexyl)urea | | |
| 91 | N-(2-chlorobenzyl)-N′-(cis-4-{[4-(dimethylamino)quinazolin-2- | 453 (M + H) | 1 |
| | yl]amino}cyclohexyl)urea | | |
| 92 | ethyl 2-({[(cis-4-{[4-(dimethylamino)quinazolin-2- | 477 (M + H) | 3 |
| | yl]amino}cyclohexyl)amino]carbonyl}amino)benzoate | | |
| 93 | N-(cis-4-{[4-(dimethylamino)quinazolin-2-yl]amino}cyclohexyl)- | 449 (M + H) | 1 |
| | N′-(2-ethoxyphenyl)urea | | |
| 94 | N-(cis-4-{[4-(dimethylamino)quinazolin-2-yl]amino}cyclohexyl)- | 475 (M + H) | 1 |
| | N′-(2-ethyl-6-isopropylphenyl)urea | | |
| 95 | N-(cis-4-{[4-(dimethylamino)quinazolin-2-yl]amino}cyclohexyl)- | 433 (M + H) | 1 |
| | N′-(2-ethylphenyl)urea | | |
| 96 | N-(cis-4-{[4-(dimethylamino)quinazolin-2-yl]amino}cyclohexyl)- | 491 (M + H) | 3 |
| | N′-[2-fluoro-3-(trifluoromethyl)phenyl]urea | | |
| 97 | N-(cis-4-{[4-(dimethylamino)quinazolin-2-yl]amino}cyclohexyl)- | 491 (M + H) | 3 |
| | N′-[2-fluro-5-(trifluoromethyl)phenyl]urea | | |
| 98 | N-(cis-4-{[4-(dimethylamino)quinazolin-2-yl]amino}cyclohexyl)- | 437 (M + H) | 3 |
| | N′-(2-fluro-5-methylphenyl)urea | | |
| 99 | N-(cis-4-{[4-(dimethylamino)quinazolin-2-yl]amino}cyclohexyl)- | 468 (M + H) | 3 |
| | N′-(2-fluoro-5-nitrophenyl)urea | | |
| 100 | N-(cis-4-{[4-(dimethylamino)quinazolin-2-yl]amino}cyclohexyl)- | 437 (M + H) | 1 |
| | N′-(2-fluorobenzyl)urea | | |
| 101 | N-(cis-4-{[4-(dimethylamino)quinazolin-2-yl]amino}cyclohexyl)- | 531 (M + H) | 1 |
| | N′-(2-iodophenyl)urea | | |
| 102 | N-(cis-4-{[4-(dimethylamino)quinazolin-2-yl]amino}cyclohexyl)- | 461 (M + H) | 1 |
| | N′-(2-isopropyl-6-methylphenyl)urea | | |
| 103 | N-(cis-4-{[4-(dimethylamino)quinazolin-2-yl]amino}cyclohexyl)- | 447 (M + H) | 1 |
| | N′-(2-isopropylphenyl)urea | | |
| 104 | N-(cis-4-{[4-(dimethylamino)quinazolin-2-yl]amino}cyclohexyl)- | 480 (M + H) | 2 |
| | N′-(2-methoxy-4-nitrophenyl)urea | | |
| 105 | N-(cis-4-{[4-(dimethylamino)quinazolin-2-yl]amino}cyclohexyl)- | 449 (M + H) | 2 |
| | N′-(2-methoxy-5-methylphenyl)urea | | |
| 106 | N-(cis-4-{[4-(dimethylamino)quinazolin-2-yl]amino}cyclohexyl)- | 480 (M + H) | 3 |
| | N′-(2-methoxy-5-nitrophenyl)urea | | |
| 107 | N-(cis-4-{[4-(dimethylamino)quinazolin-2-yl]amino}cyclohexyl)- | 464 (M + H) | 1 |
| | N′-(2-methyl-3-nitrophenyl)urea | | |
| 108 | N-(cis-4-{[4-(dimethylamino)quinazolin-2-yl]amino}cyclohexyl)- | 464 (M + H) | 1 |
| | N′-(2-methyl-4-nitrophenyl)urea | | |
| 109 | N-(cis-4-{[4-(dimethylamino)quinazolin-2-yl]amino}cyclohexyl)- | 464 (M + H) | 1 |
| | N′-(2-methyl-5-nitrophenyl)urea | | |
| 110 | N-(cis-4-{[4-(dimethylamino)quinazolin-2-yl]amino}cyclohexyl)- | 464 (M + H) | 3 |
| | N′-(2-methyl-6-nitrophenyl)urea | | |
| 111 | N-(cis-4-{[4-(dimethylamino)quinazolin-2-yl]amino}cyclohexyl)- | 433 (M + H) | 1 |
| | N′-(2-methylbenzyl)urea | | |
| 112 | N-(cis-4-{[4-(dimethylamino)quinazolin-2-yl]amino}cyclohexyl)- | 455 (M + H) | 3 |
| | N′-2-naphthylurea | | |
| 113 | N-(cis-4-{[4-(dimethylamino)quinazolin-2-yl]amino}cyclohexyl)- | 450 (M + H) | 1 |
| | N′-(2-nitrophenyl)urea | | |
| 114 | N-(cis-4-{[4-(dimethylamino)quinazolin-2-yl]amino}cyclohexyl)- | 447 (M + H) | 1 |
| | N′-(2-propylphenyl)urea | | |
| 115 | N-(cis-4-{[4-(dimethylamino)quinazolin-2-yl]amino}cyclohexyl)- | 497 (M + H) | 2 |
| | N′-(2-phenoxyphenyl)urea | | |
| 116 | N-(2-tert-butyl-6-methylphenyl)-N′-(cis-4-{[4- | 475 (M + H) | 1 |
| | (dimethylamino)quinazolin-2-yl]amino}cyclohexyl)urea | | |
| 117 | N-(2-tert-butylphenyl)-N′-(cis-4-{[4-(dimethylamino)quinazolin- | 461 (M + H) | 1 |
| | 2-yl]amino}cyclohexyl)urea | | |
| 118 | N-(cis-4-{[4-(dimethylamino)quinazolin-2-yl]amino}cyclohexyl)- | 451 (M + H) | 2 |
| | N′-[3-(methylthio)phenyl]urea | | |
| 119 | N-(cis-4-{[4-(dimethylamino)quinazolin-2-yl]amino}cyclohexyl)- | 505 (M + H) | 3 |
| | N′-{3-[(trifluoromethyl)thio]phenyl}urea | | |
| 120 | N-1,3-benzodioxol-5-yl-N′-(cis-4-{[4-(dimethylamino)quinazolin- | 449 (M + H) | 1 |
| | 2-yl]amino}cyclohexyl)urea | | |
| 121 | N-(cis-4-{[4-(dimethylamino)quinazolin-2-yl]amino}cyclohexyl)- | 495 (M + H) | 1 |
| | N′-(3,4,5-trimethoxyphenyl)urea | | |
| 122 | N-(3,4-dichlorobenzyl)-N′-(cis-4-{[4-(dimethylamino)quinazolin- | 487 (M + H) | 2 |
| | 2-yl]amino}cyclohexyl)urea | | |
| 123 | N-(3,4-difluorophenyl)-N′-(cis-4-{[4-(dimethylamino)quinazolin- | 441 (M + H) | 2 |
| | 2-yl]amino}cyclohexyl)urea | | |
| 124 | N-(3,4-dimethoxyphenyl)-N′-(cis-4-{[4- | 465 (M + H) | 1 |
| | (dimethylamino)quinazolin-2-yl]amino}cyclohexyl)urea | | |
| 125 | N-(3,5-difluorophenyl)-N′-(cis-4-{[4-(dimethylamino)quinazolin- | 441 (M + H) | 2 |
| | 2-yl]amino}cyclohexyl)urea | | |
| 126 | N-(3,5-dimethoxyphenyl)-N′-(cis-4-{[4- | 465 (M + H) | 2 |
| | (dimethylamino)quinazolin-2-yl]amino}cyclohexyl)urea | | |
| 127 | N-(cis-4-{[4-(dimethylamino)quinazolin-2-yl]amino}cyclohexyl)- | 433 (M + H) | 2 |
| | N′-(3,5-dimethylphenyl)urea | | |
| 128 | methyl 3-({[(cis-4-{[4-(dimethylamino)quinazolin-2- | 463 (M + H) | 2 |
| | yl]amino}cyclohexyl)amino]carbonyl}amino)benzoate | | |
| 129 | N-(3-chloro-2-methylphenyl)-N′-(cis-4-{[4- | 453 (M + H) | 1 |
| | (dimethylamino)quinazolin-2-yl]amino}cyclohexyl)urea | | |
| 130 | N-(3-chloro-4-fluorophenyl)-N′-(cis-4-{[4- | 457 (M + H) | 2 |
| | (dimethylamino)quinazolin-2-yl]amino}cyclohexyl)urea | | |
| 131 | N-(3-chloro-4-methoxyphenyl)-N′-(cis-4-{[4- | 469 (M + H) | 1 |
| | (dimethylamino)quinazolin-2-yl]amino}cyclohexyl)urea | | |
| 132 | N-(cis-4-{[4-(dimethylamino)quinazolin-2-yl]amino}cyclohexyl)- | 433 (M + H) | 2 |
| | N′-(3-ethylphenyl)urea | | |
| 133 | N-(cis-4-{[4-(dimethylamino)quinazolin-2-yl]amino}cyclohexyl)- | 491 (M + H) | 3 |
| | N′-[3-fluoro-5-(trifluoromethyl)phenyl]urea | | |
| 134 | N-(cis-4-{[4-(dimethylamino)quinazolin-2-yl]amino}cyclohexyl)- | 437 (M + H) | 2 |
| | N′-(3-fluorobenzyl)urea | | |
| 135 | N-(cis-4-{[4-(dimethylamino)quinazolin-2-yl]amino}cyclohexyl)- | 448(M−H) | 3 |
| | N′-(3-nitrophenyl)urea | | |
| 136 | N-(cis-4-{[4-(dimethylamino)quinazolin-2-yl]amino}cyclohexyl)- | 473 (M + H) | 3 |
| | N′-[4-(trifluoromethyl)phenyl]urea | | |
| 137 | N-(cis-4-{[4-(dimethylamino)quinazolin-2-yl]amino}cyclohexyl)- | 505 (M + H) | 3 |
| | N′-{4-[(trifluoromethyl)thio]phenyl}urea | | |
| 138 | N-(cis-4-{[4-(dimethylamino)quinazolin-2-yl]amino}cyclohexyl)- | 478 (M + H) | 3 |
| | N′-(4,5-dimethyl-2-nitrophenyl)urea | | |
| 139 | N-[4-(benzyloxy)phenyl]-N′-(cis-4-{[4- | 511 (M + H) | 3 |
| | (dimethylamino)quinazolin-2-yl]amino}cyclohexyl)urea | | |
| 140 | N-(4-benzylphenyl)-N′-(cis-4-{[4-(dimethylamino)quinazolin-2- | 495 (M + H) | 3 |
| | yl]amino}cyclohexyl)urea | | |
| 141 | N-[4-bromo-2-(trifluoromethyl)pheny]-N′-(cis-4-{[4- | 551 (M + H) | 1 |
| | (dimethylamino)quinazolin-2-yl]amino}cyclohexyl)urea | | |
| 142 | N-(4-bromo-2,6-difluorophenyl)-N′-(cis-4-{[4- | 519 (M + H) | 1 |
| | (dimethylamino)quinazolin-2-yl]amino}cyclohexyl)urea | | |
| 143 | N-(4-bromo-2-chlorophenyl)-N′-(cis-4-{[4- | 517 (M + H) | 3 |
| | (dimethylamino)quinazolin-2-yl]amino}cyclohexyl)urea | | |
| 144 | N-(4-bromobenzyl)-N′-(cis-4-{[4-(dimethylamino)quinazolin-2- | 497 (M + H) | 1 |
| | yl]amino}cyclohexyl)urea | | |
| 145 | N-[4-chloro-2-(trifluoromethyl)phenyl]-N′-(cis-4-{[4- | 507 (M + H) | 1 |
| | (dimethylamino)quinazolin-2-yl]amino}cyclohexyl)urea | | |
| 146 | N-(4-chloro-2-methylphenyl)-N′-(cis-4-{[4- | 453 (M + H) | 1 |
| | (dimethylamino)quinazolin-2-yl]amino}cyclohexyl)urea | | |
| 147 | N-(4-chloro-2-nitrophenyl)-N′-(cis-4-{[4- | 484 (M + H) | 3 |
| | (dimethylamino)quinazolin-2-yl]amino}cyclohexyl)urea | | |
| 148 | N-[4-chloro-3-(trifluoromethyl)phenyl]-N′-(cis-4-{[4- | 507 (M + H) | 3 |
| | (dimethylamino)quinazolin-2-yl]amino}cyclohexyl)urea | | |
| 149 | N-(4-cyanophenyl)-N′-(cis-4-{[4-(dimethylamino)quinazolin-2- | 430 (M + H) | 1 |
| | yl]amino}cyclohexyl)urea | | |
| 150 | N-(cis-4-{[4-(dimethylamino)quinazolin-2-yl]amino}cyclohexyl)- | 449 (M + H) | 2 |
| | N′-(4-ethoxyphenyl)urea | | |
| 151 | N-[1-(4-bromophenyl)ethyl]-N′-(cis-4-{[4- | 511 (M + H) | 1 |
| | (dimethylamino)quinazolin-2-yl]amino}cyclohexyl)urea | | |
| 152 | N-(cis-4-{[4-(dimethylamino)quinazolin-2-yl]amino}cyclohexyl)- | 489 (M + H) | 3 |
| | N′-[2-(trifluoromethoxy)phenyl]urea | | |
| 153 | N-(3-acetylphenyl)-N′-(cis-4-{[4-(dimethylamino)quinazolin-2- | 463 (M + H) | 3 |
| | yl]amino}cyclohexyl)thiourea | | |
| 154 | N-(4-acetylphenyl)-N′-(cis-4-{[4-(dimethylamino)quinazolin-2- | 463 (M + H) | 3 |
| | yl]amino}cyclohexyl)thiourea | | |
| 155 | N-[3,5-bis(trifluoromethyl)phenyl]-N′-(cis-4-{[4- | 557 (M + H) | 3 |
| | (dimethylamino)quinazolin-2-yl]amino}cyclohexyl)thiourea | | |
| 156 | N-benzyl-N′-(cis-4-{[4-(dimethylamino)quinazolin-2- | 435 (M + H) | 3 |
| | yl]amino}cyclohexyl)thiourea | | |
| 157 | N-(3-bromopheny)-N′-(cis-4-{[4-(dimethylamino)quinazolin-2- | 499 (M + H) | 3 |
| | yl]amino}cyclohexyl)thiourea | | |
| 158 | N-(4-bromophenyl)-N′-(cis-4-{[4-(dimethylamino)quinazolin-2- | 499 (M + H) | 1 |
| | yl]amino}cyclohexyl)thiourea | | |
| 159 | N-butyl-N′-(cis-4-{[4-(dimethylamino)quinazolin-2- | 401 (M + H) | 3 |
| | yl]amino}cyclohexyl)thiourea | | |
| 160 | N-(4-cyanophenyl)-N′-(cis-4-{[4-(dimethylamino)quinazolin-2- | 446 (M + H) | 1 |
| | yl]amino}cyclohexyl)thiourea | | |
| 161 | N-cyclohexyl-N′-(cis-4-{[4-(dimethylamino)quinazolin-2- | 427 (M + H) | 2 |
| | yl]amino}cyclohexyl)thiourea | | |
| 162 | N-cyclopentyl-N′-(cis-4-{[4-(dimethylamino)quinazolin-2- | 413 (M + H) | 2 |
| | yl]amino}cyclohexyl)thiourea | | |
| 163 | N-(3-chlorophenyl)-N′-(cis-4-{[4-(dimethylamino)quinazolin-2- | 455 (M + H) | 3 |
| | yl]amino}cyclohexyl)thiourea | | |
| 164 | N-(4-chlorophenyl)-N′-(cis-4-{[4-(dimethylamino)quinazolin-2- | 455 (M + H) | 2 |
| | yl]amino}cyclohexyl)thiourea | | |
| 165 | N-(2,4-dichlorophenyl)-N′-(cis-4-{[4-(dimethylamino)quinazolin- | 489 (M + H) | 1 |
| | 2-yl]amino}cyclohexyl)thiourea | | |
| 166 | N-(2,4-dimethoxyphenyl)-N′-(cis-4-{[4- | 481 (M + H) | 1 |
| | (dimethylamino)quinazolin-2-yl]amino}cyclohexyl)thiourea | | |
| 167 | N-(2,5-difluorophenyl)-N′-(cis-4-{[4-(dimethylamino)quinazolin- | 457 (M + H) | 3 |
| | 2-yl]amino}cyclohexyl)thiourea | | |
| 168 | N-(2,5-dichlorophenyl)-N′-(cis-4-{[4-(dimethylamino)quinazolin- | 489 (M + H) | 3 |
| | 2-yl]amino}cyclohexyl)thiourea | | |
| 169 | N-(cis-4-{[4-(dimethylamino)quinazolin-2-yl]amino}cyclohexyl)- | 449 (M + H) | 1 |
| | N′-(2,6-dimethylphenyl)thiourea | | |
| 170 | N-(3,4-dichlorophenyl)-N′-(cis-4-{[4-(dimethylamino)quinazolin- | 489 (M + H) | 3 |
| | 2-yl]amino}cyclohexyl)thiourea | | |
| 171 | N-(2,6-dichlorophenyl)-N′-(cis-4-{[4-(dimethylamino)quinazolin- | 489 (M + H) | 3 |
| | 2-yl]amino}cyclohexyl)thiourea | | |
| 172 | N-(cis-4-{[4-(dimethylamino)quinazolin-2-yl]amino}cyclohexyl)- | 465 (M + H) | 3 |
| | N′-(4-ethoxyphenyl)thiourea | | |
| 173 | N-(cis-4-{[4-(dimethylamino)quinazolin-2-yl]amino}cyclohexyl)- | 491 (M + H) | 1 |
| | N′-(2-ethyl-6-isoprophylphenyl)thiourea | | |
| 174 | N-(cis-4-{[4-(dimethylamino)quinazolin-2-yl]amino}cyclohexyl)- | 425 (M + H) | 3 |
| | N′-(2-furylmethyl)thiourea | | |
| 175 | N-(cis-4-{[4-(dimethylamino)quinazolin-2-yl]amino}cyclohexyl)- | 439 (M + H) | 2 |
| | N′-(4-fluorophenyl)thiourea | | |
| 176 | N-(cis-4-{[4-(dimethylamino)quinazolin-2-yl]amino}cyclohexyl)- | 429 (M + H) | 2 |
| | N′-hexylthiourea | | |
| 177 | N-(cis-4-{[4-(dimethylamino)quinazolin-2-yl]amino}cyclohexyl)- | 545 (M + H) | 3 |
| | N′-[4-(trans-4-propylcyclohexyl)phenyl]thiourea | | |
| 178 | N-(cis-4-{[4-(dimethylamino)quinazolin-2-yl]amino}cyclohexyl)- | 401 (M + H) | 2 |
| | N′-isobutylthiourea | | |
| 179 | N-(cis-4-{[4-(dimethylamino)quinazolin-2-yl]amino}cyclohexyl)- | 527 (M + H) | 2 |
| | N′-(4-methoxybiphenyl-3-yl)thiourea | | |
| 180 | N-(1,3-benzodioxol-5-ylmethyl)-N′-(cis-4-{[4- | 479 (M + H) | 2 |
| | (dimethylamino)quinazolin-2-yl]amino}cyclohexyl)thiourea | | |
| 181 | N-(cis-4-{[4-(dimethylamino)quinazolin-2-yl]amino}cyclohexyl)- | 435 (M + H) | 3 |
| | N′-(3-methylphenyl)thiourea | | |
| 182 | N-(cis-4-{[4-(dimethylamino)quinazolin-2-yl]amino}cyclohexyl)- | 467 (M + H) | 2 |
| | N′-[4-(methylthio)phenyl]thiourea | | |
| 183 | N-(cis-4-{[4-(dimethylamino)quinazolin-2-yl]amino}cyclohexyl)- | 451 (M + H) | 2 |
| | N′-(4-methoxyphenyl)thiourea | | |
| 184 | N-(cis-4-{[4-(dimethylamino)quinazolin-2-yl]amino}cyclohexyl)- | 399 (M + H) | 3 |
| | N′-(2-methylprop-2-en-1-yl)thiourea | | |
| 185 | N-(cis-4-{[4-(dimethylamino)quinazolin-2-yl]amino}cyclohexyl)- | 451 (M + H) | 1 |
| | N′-(2-methoxyphenyl)thiourea | | |
| 186 | N-(cis-4-{[4-(dimethylamino)quinazolin-2-yl]amino}cyclohexyl)- | 359 (M + H) | 3 |
| | N′-methylthiourea | | |
| 187 | N-(cis-4-{[4-(dimethylamino)quinazolin-2-yl]amino}cyclohexyl)- | 471 (M + H) | 1 |
| | N′-1-naphthylthiourea | | |
| 188 | N-(cis-4-{[4-(dimethylamino)quinazolin-2-yl]amino}cyclohexyl)- | 466 (M + H) | 3 |
| | N′-(3-nitrophenyl)thiourea | | |
| 189 | N-(cis-4-{[4-(dimethylamino)quinazolin-2-yl]amino}cyclohexyl)- | 466 (M + H) | 2 |
| | N′-(4-nitrophenyl)thiourea | | |
| 190 | N-(cis-4-{[4-(dimethylamino)quinazolin-2-yl]amino}cyclohexyl)- | 457 (M + H) | 3 |
| | N′-(1,1,3,3-tetramethylbutyl)thiourea | | |
| 191 | N-(cis-4-{[4-(dimethylamino)quinazolin-2-yl]amino}cyclohexyl)- | 421 (M + H) | 3 |
| | N′-phenylthiourea | | |
| 192 | N-(cis-4-{[4-(dimethylamino)quinazolin-2-yl]amino}cyclohexyl)- | 511 (M + H) | 2 |
| | N′-(pentafluorophenyl)thiourea | | |
| 193 | N-(cis-4-{[4-(dimethylamino)quinazolin-2-yl]amino}cyclohexyl)- | 387 (M + H) | 2 |
| | N′-propylthiourea | | |
| 194 | N-(cis-4-{[4-(dimethylamino)quinazolin-2-yl]amino}cyclohexyl)- | 489 (M + H) | 3 |
| | N′-[3-(trifluoromethyl)phenyl]thiourea | | |
| 195 | N-(cis-4-{[4-(dimethylamino)quinazolin-2-yl]amino}cyclohexyl)- | 511 (M + H) | 1 |
| | N′-(3,4,5-trimethoxyphenyl)thiourea | | |
| 196 | N-(cis-4-{[4-(dimethylamino)quinazolin-2-yl]amino}cyclohexyl)- | 429 (M + H) | 3 |
| | N′-(tetrahydrofuran-2-ylmethyl)thiourea | | |
| 197 | N-(cis-4-{[4-(dimethylamino)quinazolin-2-yl]amino}cyclohexyl)- | 435 (M + H) | 2 |
| | N′-(4-methylphenyl)thiourea | | |
| 198 | N-(cis-4-{[4-(dimethylamino)quinazolin-2-yl]amino}cyclohexyl)- | 435 (M + H) | 3 |
| | N′-(2-methylphenyl)thiourea | | |
| 199 | N-(tert-butyl)-N′-(cis-4-{[4-(dimethylamino)quinazolin-2- | 401 (M + H) | 3 |
| | yl]amino}cyclohexyl)thiourea | | |
| 200 | N-1-adamantyl-N′-(cis-4-{[4-(dimethylamino)quinazolin-2- | 479 (M + H) | 3 |
| | yl]amino}cyclohexyl)thiourea | | |
| 201 | N-(2-bromophenyl)-N′-(cis-4-{[4-(dimethylamino)quinazolin-2- | 499 (M + H) | 3 |
| | yl]amino}cyclohexyl)thiourea | | |
| 202 | N-(2-chlorophenyl)-N′-(cis-4-{[4-(dimethylamino)quinazolin-2- | 455 (M + H) | 3 |
| | yl]amino}cyclohexyl)thiourea | | |
| 203 | N-(cis-4-{[4-(dimethylamino)quinazolin-2-yl]amino}cyclohexyl)- | 449 (M + H) | 3 |
| | N′-(2-phenylethyl)thiourea | | |
| 204 | N-(3,4-dimethoxyphenyl)-N′-(cis-4-{[4- | 481 (M + H) | 1 |
| | (dimethylamino)quinazolin-2-yl]amino}cyclohexyl)thiourea | | |
| 205 | N-(cis-4-{[4-(dimethylamino)quinazolin-2-yl]amino}cyclohexyl)- | 449 (M + H) | 2 |
| | N′-(4-ethylphenyl)thiourea | | |
| 206 | N-(cis-4-{[4-(dimethylamino)quinazolin-2-yl]amino}cyclohexyl)- | 467 (M + H) | 2 |
| | N′-[2-(methylthio)phenyl]thiourea | | |
| 207 | N-(cis-4-{[4-(dimethylamino)quinazolin-2-yl]amino}cyclohexyl)- | 505 (M + H) | 2 |
| | N′-[2-(trifluoromethoxy)phenyl]thiourea | | |
| 208 | N-(cis-4-{[4-(dimethylamino)quinazolin-2-yl]amino}cyclohexyl)- | 489 (M + H) | 3 |
| | N′-[2-(trifluoromethyl)phenyl]thiourea | | |
| 209 | N-(cis-4-{[4-(dimethylamino)quinazolin-2-yl]amino}cyclohexyl)- | 475 (M + H) | 2 |
| | N′-(2,3,4-trifluorophenyl)thiourea | | |
| 210 | N-(2,3-dichlorophenyl)-N′-(cis-4-{[4-(dimethylamino)quinazolin- | 489 (M + H) | 3 |
| | 2-yl]amino}cyclohexyl)thiourea | | |
| 211 | N-(2,4-difluorophenyl)-N′-(cis-4-{[4-(dimethylamino)quinazolin- | 457 (M + H) | 3 |
| | 2-yl]amino}cyclohexyl)thiourea | | |
| 212 | N-(2,5-dimethoxyphenyl)-N′-(cis-4-{[4- | 481 (M + H) | 2 |
| | (dimethylamino)quinazolin-2-yl]amino}cyclohexyl)thiourea | | |
| 213 | N-(2,6-difluorophenyl)-N′-(cis-4-{[4-(dimethylamino)quinazolin- | 457 (M + H) | 3 |
| | 2-yl]amino}cyclohexyl)thiourea | | |
| 214 | N-(2-chloro-4-nitrophenyl)-N′-(cis-4-{[4- | 500 (M + H) | 2 |
| | (dimethylamino)quinazolin-2-yl]amino}cyclohexyl)thiourea | | |
| 215 | N-[2-(difluoromethoxy)phenyl]-N′-(cis-4-{[4- | 487 (M + H) | 3 |
| | (dimethylamino)quinazolin-2-yl]amino}cyclohexyl)thiourea | | |
| 216 | N-(cis-4-{[4-(dimethylamino)quinazolin-2-yl]amino}cyclohexyl)- | 449 (M + H) | 1 |
| | N′-(2-ethylphenyl)thiourea | | |
| 217 | N-(cis-4-{[4-(dimethylamino)quinazolin-2-yl]amino}cyclohexyl)- | 507 (M + H) | 3 |
| | N′-[2-fluoro-5-(trifluoromethyl)phenyl]thiourea | | |
| 218 | N-(cis-4-{[4-(dimethylamino)quinazolin-2-yl]amino}cyclohexyl)- | 439 (M + H) | 3 |
| | N′-(2-fluorophenyl)thiourea | | |
| 219 | N-(cis-4-{[4-(dimethylamino)quinazolin-2-yl]amino}cyclohexyl)- | 547 (M + H) | 2 |
| | N′-(2-iodophenyl)thiourea | | |
| 220 | N-(cis-4-{[4-(dimethylamino)quinazolin-2-yl]amino}cyclohexyl)- | 496 (M + H) | 1 |
| | N′-(2-methoxy-4-nitrophenyl)thiourea | | |
| 221 | N-(cis-4-{[4-(dimethylamino)quinazolin-2-yl]amino}cyclohexyl)- | 465 (M + H) | 1 |
| | N′-(2-methoxy-5-methylphenyl)thiourea | | |
| 222 | N-(cis-4-{[4-(dimethylamino)quinazolin-2-yl]amino}cyclohexyl)- | 521 (M + H) | 3 |
| | N′-{3-[(trifluoromethyl)thio]phenyl}thiourea | | |
| 223 | N-(3,5-dichlorophenyl)-N′-(cis-4-{[4-(dimethylamino)quinazolin- | 489 (M + H) | 3 |
| | 2-yl]amino}cyclohexyl)thiourea | | |
| 224 | N-(3,5-difluorophenyl)-N′-(cis-4-{[4-(dimethylamino)quinazolin- | 457 (M + H) | 3 |
| | 2-yl]amino}cyclohexyl)thiourea | | |
| 225 | N-(3-cyanophenyl)-N′-(cis-4-{[4-(dimethylamino)quinazolin-2- | 446 (M + H) | 3 |
| | yl]amino}cyclohexyl)thiourea | | |
| 226 | N-(cis-4-{[4-(dimethylamino)quinazolin-2-yl]amino}cyclohexyl)- | 439 (M + H) | 3 |
| | N′-(3-fluorophenyl)thiourea | | |
| 227 | N-(cis-4-{[4-(dimethylamino)quinazolin-2-yl]amino}cyclohexyl)- | 547 (M + H) | 2 |
| | N′-(3-iodophenyl)thiourea | | |
| 228 | N-(cis-4-{[4-(dimethylamino)quinazolin-2-yl]amino}cyclohexyl)- | 451 (M + H) | 2 |
| | N′-(3-methoxyphenyl)thiourea | | |
| 229 | N-[4-(difluoromethoxy)phenyl]-N′-(cis-4-{[4- | 487 (M + H) | 2 |
| | (dimethylamino)quinazolin-2-yl]amino}cyclohexyl)thiourea | | |
| 230 | N-(cis-4-{[4-(dimethylamino)quinazolin-2-yl]amino}cyclohexyl)- | 505 (M + H) | 3 |
| | N′-[4-(trifluoromethoxy)phenyl]thiourea | | |
| 231 | N-(cis-4-{[4-(dimethylamino)quinazolin-2-yl]amino}cyclohexyl)- | 489 (M + H) | 2 |
| | N′-[4-(trifluoromethyl)phenyl]thiourea | | |
| 232 | N-(cis-4-{[4-(dimethylamino)quinazolin-2-yl]amino}cyclohexyl)- | 521 (M + H) | 3 |
| | N′-{4-[(trifluoromethyl)thio]phenyl}thiourea | | |
| 233 | N-(4-bromo-2-chlorophenyl)-N′-(cis-4-{[4- | 533 (M + H) | 1 |
| | (dimethylamino)quinazolin-2-yl]amino}cyclohexyl)thiourea | | |
| 234 | N-(4-bromo-2-fluorophenyl)-N′-(cis-4-{[4- | 517 (M + H) | 3 |
| | (dimethylamino)quinazolin-2-yl]amino}cyclohexyl)thiourea | | |
| 235 | N-[4-chloro-3-(trifluoromethyl)phenyl]-N′-(cis-4-{[4- | 523 (M + H) | 3 |
| | (dimethylamino)quinazolin-2-yl]amino}cyclohexyl)thiourea | | |
| 236 | N-(cis-4-{[4-(dimethylamino)quinazolin-2-yl]amino}cyclohexyl)- | 507 (M + H) | 3 |
| | N′-[4-fluoro-3-(trifluoromethyl)phenyl]thiourea | | |
| 237 | N-(cis-4-{[4-(dimethylamino)quinazolin-2-yl]amino}cyclohexyl)- | 547 (M + H) | 1 |
| | N′-(4-iodophenyl)thiourea | | |
| 238 | N-(5-chloro-2-methylphenyl)-N′-(cis-4-{[4- | 469 (M + H) | 2 |
| | (dimethylamino)quinazolin-2-yl]amino}cyclohexyl)thiourea | | |
| 239 | N-[(1S,4R)-bicyclo[2,2,1]hept-2-yl]-N′-(cis-4-{[4- | 439 (M + H) | 2 |
| | (dimethylamino)quinazolin-2-yl]amino}cyclohexyl)thiourea | | |
| 240 | tert-butyl [4-({[(cis-4-{[4-(dimethylamino)quinazolin-2- | 536 (M + H) | 3 |
| | yl]amino}cyclohexyl)amino]carbonothioyl}amino)phenyl]- | | |
| | carbamate | | |
| 241 | N-[2-(3,4-dimethoxyphenyl)ethyl]-N′-(cis-4-{[4- | 509 (M + H) | 3 |
| | (dimethylamino)quinazolin-2-yl]amino}cyclohexyl)thiourea | | |
| 242 | N-[2-(4-chlorophenyl)ethyl]-N′-(cis-4-{[4- | 483 (M + H) | 2 |
| | (dimethylamino)quinazolin-2-yl]amino}cyclohexyl)thiourea | | |
| 243 | N-(cis-4-{[4-(dimethylamino)quinazolin-2-yl]amino}cyclohexyl)- | 557 (M + H) | 3 |
| | N′-(2,3,4,5-tetrachlorophenyl)thiourea | | |
| 244 | N-(cis-4-{[4-(dimethylamino)quinazolin-2-yl]amino}cyclohexyl)- | 523 (M + H) | 3 |
| | N′-(2,4,5-trichlorophenyl)thiourea | | |
| 245 | N-(cis-4-{[4-(dimethylamino)quinazolin-2-yl]amino}cyclohexyl)- | 654 (M + H) | 1 |
| | N′-(2,4,6-tribromophenyl)thiourea | | |
| 246 | N-(cis-4-{[4-(dimethylamino)quinazolin-2-yl]amino}cyclohexyl)- | 523 (M + H) | 1 |
| | N′-(2,4,6-trichlorophenyl)thiourea | | |
| 247 | N-(cis-4-{[4-(dimethylamino)quinazolin-2-yl]amino}cyclohexyl)- | 475 (M + H) | 3 |
| | N′-(2,4,6-trifluorophenyl)thiourea | | |
| 248 | N-(cis-4-{[4-(dimethylamino)quinazolin-2-yl]amino}cyclohexyl)- | 463 (M + H) | 1 |
| | N′-mesitylthiourea | | |
| 249 | N-(cis-4-{[4-(dimethylamino)quinazolin-2-yl]amino}cyclohexyl)- | 449 (M + H) | 1 |
| | N′-(2,4-dimethylphenyl)thiourea | | |
| 250 | N-(2,6-diethylphenyl)-N′-(cis-4-{[4-(dimethylamino)quinazolin- | 477 (M + H) | 1 |
| | 2-yl]amino}cyclohexyl)thiourea | | |
| 251 | N-(2,6-diisopropylphenyl)-N′-(cis-4-{[4- | 505 (M + H) | 2 |
| | (dimethylamino)quinazolin-2-yl]amino}cyclohexyl)thiourea | | |
| 252 | N-(2-bromo-4-methylphenyl)-N′-(cis-4-{[4- | 513 (M + H) | 1 |
| | (dimethylamino)quinazolin-2-yl]amino}cyclohexyl)thiourea | | |
| 253 | N-[2-chloro-5-(trifluoromethyl)phenyl]-N′-(cis-4-{[4- | 523 (M + H) | 3 |
| | (dimethylamino)quinazolin-2-yl]amino}cyclohexyl)thiourea | | |
| 254 | N-(2-chlorobenzyl)-N′-(cis-4-{[4-(dimethylamino)quinazolin-2- | 469 (M + H) | 1 |
| | yl]amino}cyclohexyl)thiourea | | |
| 255 | N-(cis-4-{[4-(dimethylamino)quinazolin-2-yl]amino}cyclohexyl)- | 463 (M + H) | 1 |
| | N′-(2-ethyl-6-methylphenyl)thiourea | | |
| 256 | N-(cis-4-{[4-(dimethylamino)quinazolin-2-yl]amino}cyclohexyl)- | 463 (M + H) | 1 |
| | N′-(2-isopropylphenyl)thiourea | | |
| 257 | N-(cis-4-{[4-(dimethylamino)quinazolin-2-yl]amino}cyclohexyl)- | 467 (M + H) | 3 |
| | N′-[3-(methylthio)phenyl]thiourea | | |
| 258 | N-(3,4-dichlorobenzyl)-N′-(cis-4-{[4-(dimethylamino)quinazolin- | 503 (M + H) | 3 |
| | 2-yl]amino}cyclohexyl)thiourea | | |
| 259 | N-(3,5-dimethoxyphenyl)-N′-(cis-4-{[4- | 481 (M + H) | 2 |
| | (dimethylamino)quinazolin-2-yl]amino}cyclohexyl)thiourea | | |
| 260 | N-(cis-4-{[4-(dimethylamino)quinazolin-2-yl]amino}cyclohexyl)- | 449 (M + H) | 2 |
| | N′-(3,5-dimethylphenyl)thiourea | | |
| 261 | N-[3-(benzyloxy)phenyl]-N′-(cis-4-{[4- | 527 (M + H) | 3 |
| | (dimethylamino)quinazolin-2-yl]amino}cyclohexyl)-thiourea | | |
| 262 | N-(3-chloro-4-methylphenyl)-N′-(cis-4-{[4- | 469 (M + H) | 2 |
| | (dimethylamino)quinazolin-2-yl]amino}cyclohexyl)thiourea | | |
| 263 | methyl 3-({[(cis-4-{[4-(dimethylamino)quinazolin-2- | 479 (M + H) | 1 |
| | yl]amino}cyclohexyl)amino]carbonothioyl}amino)benzoate | | |
| 264 | N-(cis-4-{[4-(dimethylamino)quinazolin-2-yl]amino}cyclohexyl)- | 463 (M + H) | 3 |
| | N′-(3-phenylpropyl)thiourea | | |
| 265 | N-[4-(benzyloxy)phenyl]-N′-(cis-4-{[4- | 527 (M + H) | 3 |
| | (dimethylamino)quinazolin-2-yl]amino}cyclohexyl)thiourea | | |
| 266 | N-(4-bromo-2,6-dimethylphenyl)-N′-(cis-4-{[4- | 527 (M + H) | 1 |
| | (dimethylamino)quinazolin-2-yl]amino}cyclohexyl)thiourea | | |
| 267 | N-(4-bromo-2-methylphenyl)-N′-(cis-4-{[4- | 513 (M + H) | 1 |
| | (dimethylamino)quinazolin-2-yl]amino}cyclohexyl)thiourea | | |
| 268 | N-[4-bromo-2-(trifluoromethyl)phenyl]-N′-(cis-4-{[4- | 567 (M + H) | 1 |
| | (dimethylamino)quinazolin-2-yl]amino}cyclohexyl)thiourea | | |
| 269 | N-(4-chloro-2-methylphenyl)-N′-(cis-4-{[4- | 469 (M + H) | 1 |
| | (dimethylamino)quinazolin-2-yl]amino}cyclohexyl)thiourea | | |
| 270 | N-(4-chloro-3-nitrophenyl)-N′-(cis-4-{[4- | 500 (M + H) | 3 |
| | (dimethylamino)quinazolin-2-yl]amino}cyclohexyl)thiourea | | |
| 271 | N-(4-chlorobenzyl)-N′-(cis-4-{[4-(dimethylamino)quinazolin-2- | 469 (M + H) | 3 |
| | yl]amino}cyclohexyl)thiourea | | |
| 272 | ethyl 4-({[(cis-4-{[4-(dimethylamino)quinazolin-2- | 493 (M + H) | 3 |
| | yl]amino}cyclohexyl)amino]carbonothioyl}amino)benzoate | | |
| 273 | N-(cis-4-{[4-(dimethylamino)quinazolin-2-yl]amino}cyclohexyl)- | 467 (M + H) | 2 |
| | N′-[1-(4-fluorophenyl)ethyl]thiourea | | |
| 274 | N-(cis-4-{[4-(dimethylamino)quinazolin-2-yl]amino}cyclohexyl)- | 453 (M + H) | 2 |
| | N′-(4-fluorobenzyl)thiourea | | |
| 275 | N-(cis-4-{[4-(dimethylamino)quinazolin-2-yl]amino}cyclohexyl)- | 463 (M + H) | 2 |
| | N′-(4-isopropylphenyl)thiourea | | |
| 276 | N-(cis-4-{[4-(dimethylamino)quinazolin-2-yl]amino}cyclohexyl)- | 496 (M + H) | 3 |
| | N′-(4-methoxy-2-nitrophenyl)thiourea | | |
| 277 | N-(cis-4-{[4-(dimethylamino)quinazolin-2-yl]amino}cyclohexyl)- | 465 (M + H) | 2 |
| | N′-(4-methoxybenzyl)thiourea | | |
| 278 | methyl 4-({[(cis-4-{[4-(dimethylamino)quinazolin-2- | 479 (M + H) | 2 |
| | yl]amino}cyclohexyl)amino]carbonothioyl}amino)benzoate | | |
| 279 | N-(cis-4-{[4-(dimethylamino)quinazolin-2-yl]amino}cyclohexyl)- | 480 (M + H) | 3 |
| | N′-(4-methyl-2-nitrophenyl)thiourea | | |
| 280 | N-(cis-4-{[4-(dimethylamino)quinazolin-2-yl]amino}cyclohexyl)- | 449 (M + H) | 3 |
| | N′-(4-methylbenzyl)thiourea | | |
| 281 | N-(4-butylphenyl)-N′-(cis-4-{[4-(dimethylamino)quinazolin-2- | 477 (M + H) | 3 |
| | yl]amino}cyclohexyl)thiourea | | |
| 282 | N-(5-chloro-2-methoxyphenyl)-N′-(cis-4-{[4- | 485 (M + H) | 3 |
| | (dimethylamino)quinazolin-2-yl]amino}cyclohexyl)thiourea | | |
| 283 | N-(cis-4-{[4-(dimethylamino)quinazolin-2-yl]amino}cyclohexyl)- | 449 (M + H) | 2 |
| | N′-(1-phenylethyl)thiourea | | |
| 284 | N-(cis-4-{[4-(dimethylamino)quinazolin-2-yl]amino}cyclohexyl)- | 511 (M + H) | 2 |
| | N′-(diphenylmethyl)thiourea | | |
| 285 | N-cyclododecyl-N′-(cis-4-{[4-(dimethylamino)quinazolin-2- | 511 (M + H) | 3 |
| | yl]amino}cyclohexyl)thiourea | | |
| 286 | N-(cyclohexylmethyl)-N′-(cis-4-{[4-(dimethylamino)quinazolin- | 441 (M + H) | 2 |
| | 2-yl]amino}cyclohexyl)thiourea | | |
| 287 | N-cyclooctyl-N′-(cis-4-{[4-(dimethylamino)quinazolin-2- | 455 (M + H) | 2 |
| | yl]amino}cyclohexyl)thiourea | | |
| 288 | N-cyclopropyl-N′-(cis-4-{[4-(dimethylamino)quinazolin-2- | 385 (M + H) | 2 |
| | yl]amino}cyclohexyl)thiourea | | |
| 289 | N-(cis-4-{[4-(dimethylamino)quinazolin-2-yl]amino}cyclohexyl)- | 485 (M + H) | 1 |
| | N′-(1-naphthylmethyl)thiourea | | |
| 290 | N-(cis-4-{[4-(dimethylamino)quinazolin-2-yl]amino}cyclohexyl)- | 525 (M + H) | 2 |
| | N′-(2,2-diphenylethyl)thiourea | | |
| 291 | N-(cis-4-{[4-(dimethylamino)quinazolin-2-yl]amino}cyclohexyl)- | 557 (M + H) | 3 |
| | N′-(2,3,5,6-tetrachlorophenyl)thiourea | | |
| 292 | N-(2,3-dimethoxybenzyl)-N′-(cis-4-{[4- | 495 (M + H) | 1 |
| | (dimethylamino)quinazolin-2-yl]amino}cyclohexyl)thiourea | | |
| 293 | N-(cis-4-{[4-(dimethylamino)quinazolin-2-yl]amino}cyclohexyl)- | 463 (M + H) | 1 |
| | N′-(2,4,5-trimethylphenyl)thiourea | | |
| 294 | N-(2,4-dichlorobenzyl)-N′-(cis-4-{[4-(dimethylamino)quinazolin- | 503 (M + H) | 3 |
| | 2-yl]amino}cyclohexyl)thiourea | | |
| 295 | N-(2,5-dibromophenyl)-N′-(cis-4-{[4-(dimethylamino)quinazolin- | 577 (M + H) | 3 |
| | 2-yl]amino}cyclohexyl)thiourea | | |
| 296 | N-[2-(2,5-dimethoxyphenyl)ethyl]-N′-(cis-4-{[4- | 509 (M + H) | 2 |
| | (dimethylamino)quinazolin-2-yl]amino}cyclohexyl)thiourea | | |
| 297 | N-biphenyl-2-yl-N′-(cis-4-{[4-(dimethylamino)quinazolin-2- | 497 (M + H) | 1 |
| | yl]amino}cyclohexyl)thiourea | | |
| 298 | N-(2-chloro-5-nitrophenyl)-N′-(cis-4-{[4- | 500 (M + H) | 3 |
| | (dimethylamino)quinazolin-2-yl]amino}cyclohexyl)thiourea | | |
| 299 | N-(2-cyanophenyl)-N′-(cis-4-{[4-(dimethylamino)quinazolin-2- | 446 (M + H) | 3 |
| | yl]amino}cyclohexyl)thiourea | | |
| 300 | N-(cis-4-{[4-(dimethylamino)quinazolin-2-yl]amino}cyclohexyl)- | 453 (M + H) | 2 |
| | N′-(2-fluorobenzyl)thiourea | | |
| 301 | N-(cis-4-{[4-(dimethylamino)quinazolin-2-yl]amino}cyclohexyl)- | 496 (M + H) | 3 |
| | N′-(2-methoxy-5-nitrophenyl)thiourea | | |
| 302 | N-(cis-4-{[4-(dimethylamino)quinazolin-2-yl]amino}cyclohexyl)- | 480 (M + H) | 1 |
| | N′-(2-methyl-4-nitrophenyl)thiourea | | |
| 303 | N-(cis-4-{[4-(dimethylamino)quinazolin-2-yl]amino}cyclohexyl)- | 449 (M + H) | 2 |
| | N′-(2-methylbenzyl)thiourea | | |
| 304 | N-(3,4-dimethoxybenzyl)-N′-(cis-4-{[4- | 495 (M + H) | 3 |
| | (dimethylamino)quinazolin-2-yl]amino}cyclohexyl)thiourea | | |
| 305 | N-(3-chlorobenzyl)-N′-(cis-4-{[4-(dimethylamino)quinazolin-2- | 469 (M + H) | 1 |
| | yl]amino}cyclohexyl)thiourea | | |
| 306 | ethyl 3-({[(cis-4-{[4-(dimethylamino)quinazolin-2- | 493 (M + H) | 1 |
| | yl]amino}cyclohexyl)amino]carbonothioyl}amino)benzoate | | |
| 307 | N-(cis-4-{[4-(dimethylamino)quinazolin-2-yl]amino}cyclohexyl)- | 449 (M + H) | 2 |
| | N′-(3-ethylphenyl)thiourea | | |
| 308 | N-(cis-4-{[4-(dimethylamino)quinazolin-2-yl]amino}cyclohexyl)- | 453 (M + H) | 2 |
| | N′-(3-fluorobenzyl)thiourea | | |
| 309 | N-(cis-4-{[4-(dimethylamino)quinazolin-2-yl]amino}cyclohexyl)- | 465 (M + H) | 2 |
| | N′-(3-methoxybenzyl)thiourea | | |
| 310 | N-(cis-4-{[4-(dimethylamino)quinazolin-2-yl]amino}cyclohexyl)- | 449 (M + H) | 2 |
| | N′-(3-methylbenzyl)thiourea | | |
| 311 | N-(4-bromo-3-chlorophenyl)-N′-(cis-4-{[4- | 533 (M + H) | 3 |
| | (dimethylamino)quinazolin-2-yl]amino}cyclohexyl)thiourea | | |
| 312 | N-(4-bromo-3-methylphenyl)-N′-(cis-4-{[4- | 513 (M + H) | 3 |
| | (dimethylamino)quinazolin-2-yl]amino}cyclohexyl)thiourea | | |
| 313 | 4-({[(cis-4-{[4-(dimethylamino)quinazolin-2- | 465 (M + H) | 3 |
| | yl]amino}cyclohexyl)amino]carbonothioyl}amino)benzoic acid | | |
| 314 | N-[4-chloro-2-(trifluoromethyl)phenyl]-N′-(cis-4-{[4- | 523 (M + H) | 1 |
| | (dimethylamino)quinazolin-2-yl]amino}cyclohexyl)thiourea | | |
| 315 | N-(4-decylphenyl)-N′-(cis-4-{[4-(dimethylamino)quinazolin-2- | 561 (M + H) | 3 |
| | yl]amino}cyclohexyl)thiourea | | |
| 316 | N-(cis-4-{[4-(dimethylamino)quinazolin-2-yl]amino}cyclohexyl)- | 453 (M + H) | 1 |
| | N′-(4-fluoro-2-methylphenyl)thiourea | | |
| 317 | N-(cis-4-{[4-(dimethylamino)quinazolin-2-yl]amino}cyclohexyl)- | 558 (M + H) | 3 |
| | N′-[4-(4-nitrophenoxy)phenyl]thiourea | | |
| 318 | N-(cis-4-{[4-(dimethylamino)quinazolin-2-yl]amino}cyclohexyl)- | 574 (M + H) | 3 |
| | N′-{4-[(4-nitrophenyl)thio]phenyl}thiourea | | |
| 319 | 4-({[(cis-4-{[4-(dimethylamino)quinazolin-2-yl]amino}- | 500 (M + H) | 3 |
| | cyclohexyl)amino]carbonothioyl}amino)benzenesulfonamide | | |
| 320 | N-(cis-4-{[4-(dimethylamino)quinazolin-2-yl]amino}cyclohexyl)- | 465 (M + H) | 1 |
| | N′-(4-methoxy-2-methylphenyl)thiourea | | |
| 321 | N-{[(cis-4-{[4-(dimethylamino)quinazolin-2-yl]amino}- | 479 (M + H) | 3 |
| | cyclohexyl)amino]carbonothioyl}-4-methoxybenzamide | | |
| 322 | N-(cis-4-{[4-(dimethylamino)quinazolin-2-yl]amino}cyclohexyl)- | 463 (M + H) | 3 |
| | N′-[2-(4-methylphenyl)ethyl]thiourea | | |
| 323 | N-(cis-4-{[4-(dimethylamino)quinazolin-2-yl]amino}cyclohexyl)- | 513 (M + H) | 3 |
| | N′-(4-phenoxyphenyl)thiourea | | |
| 324 | N-(5-chloro-2,4-dimethoxyphenyl)-N′-(cis-4-{[4- | 515 (M + H) | 1 |
| | (dimethylamino)quinazolin-2-yl]amino}cyclohexyl)thiourea | | |
| 325 | N-(2,3-dihydro-1H-inden-5-yl)-N′-(cis-4-{[4- | 461 (M + H) | 2 |
| | (dimethylamino)quinazolin-2-yl]amino}cyclohexyl)thiourea | | |
| 326 | (2E)-N-{[(cis-4-{[4-(dimethylamino)quinazolin-2- | 475 (M + H) | 3 |
| | yl]amino}cyclohexyl)amino]carbonothioyl}-3-phenylacrylamide | | |
| 327 | N-[(2E)-but-2-en-1-yl]-N′-(cis-4-{[4-(dimethylamino)quinazolin- | 399 (M + H) | 3 |
| | 2-yl]amino}cyclohexyl)thiourea | | |
| 328 | N-cycloheptyl-N′-(cis-4-{[4-(dimethylamino)quinazolin-2- | 441 (M + H) | 2 |
| | yl]amino}cyclohexyl)thiourea | | |
| 329 | N-(cis-4-{[4-(dimethylamino)quinazolin-2-yl]amino}cyclohexyl)- | 449 (M + H) | 1 |
| | N′-[(1R)-1-phenylethyl]thiourea | | |
| 330 | butyl 2-({[(cis-4-{[4-(dimethylamino)quinazolin-2- | 505 (M + H) | 3 |
| | yl]amino}cyclohexyl)amino]carbonyl}amino)benzoate | | |
| 331 | dimethyl 5-({[(cis-4-{[4-(dimethylamino)quinazolin-2- | 521 (M + H) | 3 |
| | yl]amino}cyclohexyl)amino]carbonyl}amino)isophthalate | | |
| 332 | N-(cis-4-{[4-(dimethylamino)quinazolin-2-yl]amino}cyclohexyl)- | 489 (M + H) | 3 |
| | N′-[4-(trifluoromethoxy)phenyl]urea | | |
| 333 | N-(4-bromo-2,6-dimethylphenyl)-N′-(cis-4-{[4- | 511 (M + H) | 1 |
| | (dimethylamino)quinazolin-2-yl]amino}cyclohexyl)urea | | |
| 334 | N-(4-bromo-2-methylphenyl)-N′-(cis-4-{[4- | 497 (M + H) | 1 |
| | (dimethylamino)quinazolin-2-yl]amino}cyclohexyl)urea | | |
| 335 | N-(cis-4-{[4-(dimethylamino)quinazolin-2-yl]amino}cyclohexyl)- | 535 (M + H) | 3 |
| | N′-(2,2,4,4-tetrafluoro-4H-1,3-benzodioxin-6-yl)urea | | |
| 336 | ethyl N-{[(cis-4-{[4-(dimethylamino)quinazolin-2- | 505 (M + H) | 1 |
| | yl]amino}cyclohexyl)amino]carbonyl}phenylalaninate | | |
| 337 | N-(cis-4-{[4-(dimethylamino)quinazolin-2-yl]amino}cyclohexyl)- | 439 (M + H) | 2 |
| | N′-[2-(2-thienyl)ethyl]urea | | |
| 338 | N-(2,3-dihydro-1,4-benzodioxin-6-yl)-N′-(cis-4-{[4- | 463 (M + H) | 1 |
| | (dimethylamino)quinazolin-2-yl]amino}cyclohexyl)urea | | |
| 339 | N-(2,6-dibromo-4-isopropylphenyl)-N′-(cis-4-{[4- | 603 (M + H) | 1 |
| | (dimethylamino)quinazolin-2-yl]amino}cyclohexyl)urea | | |
| 340 | N-(2-cyanophenyl)-N′-(cis-4-{[4-(dimethylamino)quinazolin-2- | 430 (M + H) | 3 |
| | yl]amino}cyclohexyl)urea | | |
| 341 | N-(cis-4-{[4-(dimethylamino)quinazolin-2-yl]amino}cyclohexyl)- | 411 (M + H) | 3 |
| | N′-2-thienylurea | | |
| 342 | N-[3-(cyclopentyloxy)-4-methoxyphenyl]-N′-(cis-4-{[4- | 519 (M + H) | 1 |
| | (dimethylamino)quinazolin-2-yl]amino}cyclohexyl)urea | | |
| 343 | N-(3,4-dihydro-2H-1,5-benzodioxepin-7-yl)-N′-(cis-4-{[4- | 477 (M + H) | 1 |
| | (dimethylamino)quinazolin-2-yl]amino}cyclohexyl)urea | | |
| 344 | N-(cis-4-{[4-(dimethylamino)quinazolin-2-yl]amino}cyclohexyl)- | 411 (M + H) | 3 |
| | N′-3-thienylurea | | |
| 345 | N-(4-tert-butylphenyl)-N′-(cis-4-{[4-(dimethylamino)quinazolin- | 461 (M + H) | 3 |
| | 2-yl]amino}cyclohexyl)urea | | |
| 346 | N-(4-butyl-2-methylphenyl)-N′-(cis-4-{[4- | 475 (M + H) | 1 |
| | (dimethylamino)quinazolin-2-yl]amino}cyclohexyl)urea | | |
| 347 | N-(cis-4-{[4-(dimethylamino)quinazolin-2-yl]amino}cyclohexyl)- | 477 (M + H) | 1 |
| | N′-[5-methyl-2-(trifluoromethyl)-3-furyl]urea | | |
| 348 | N-(cis-4-{[4-(dimethylamino)quinazolin-2-yl]amino}cyclohexyl)- | 487 (M + H) | 3 |
| | N′-(5-phenyl-2-thienyl)urea | | |
| 349 | N-(cis-4-{[4-(dimethylamino)quinazolin-2-yl]amino}cyclohexyl)- | 481 (M + H) | 2 |
| | N′-(6-fluoro-4H-1,3-benzodioxin-8-yl)urea | | |
| 350 | benzyl 4-({[(cis-4-{[4-(dimethylamino)quinazolin-2-yl]amino}- | 546 (M + H) | 3 |
| | cyclohexyl)amino]carbonyl}amino)piperidine-1-carboxylate | | |
| 351 | N-[4-(dimethylamino)phenyl]-N′-(cis-4-{[4- | 448 (M + H) | 3 |
| | (dimethylamino)quinazolin-2-yl]amino}cyclohexyl)urea | | |
| 352 | N-(2,6-dichloropyridin-4-yl)-N′-(cis-4-{[4- | 474 (M + H) | 3 |
| | (dimethylamino)quinazolin-2-yl]amino}cyclohexyl)urea | | |
| 353 | N-(cis-4-{[4-(dimethylamino)quinazolin-2-yl]amino}cyclohexyl)- | 424 (M + H) | 2 |
| | N′-(3,5-dimethylisoxazol-4-yl)urea | | |
| 354 | N-(cis-4-{[4-(dimethylamino)quinazolin-2-yl]amino}cyclohexyl)- | 486 (M + H) | 1 |
| | N′-(3-methyl-5-phenylisoxazol-4-yl)urea | | |
| 355 | N-(cis-4-{[4-(dimethylamino)quinazolin-2-yl]amino}cyclohexyl)- | 486 (M + H) | 1 |
| | N′-(5-methyl-3-phenylisoxazol-4-yl)urea | | |
| 356 | N-(cis-4-{[4-(dimethylamino)quinazolin-2-yl]amino}cyclohexyl)- | 383 (M + H) | 3 |
| | N′-prop-2-yn-1-ylthiourea | | |
| 357 | N-(cis-4-{[4-(dimethylamino)quinazolin-2-yl]amino}cyclohexyl)- | 568 (M + H) | 3 |
| | N′-[4-(piperidin-1-ylsulfonyl)phenyl]thiourea | | |
| 358 | N-(2-cyclohex-1-en-1-ylethyl)-N′-(cis-4-{[4- | 453 (M + H) | 2 |
| | (dimethylamino)quinazolin-2-yl]amino}cyclohexyl)thiourea | | |
| 359 | N-(cis-4-{[4-(dimethylamino)quinazolin-2-yl]amino}cyclohexyl)- | 449 (M + H) | 1 |
| | N′-(2,3-dimethylphenyl)thiourea | | |
| 360 | N-(2,4-dibromo-6-fluorophenyl)-N′-(cis-4-{[4- | 595 (M + H) | 1 |
| | (dimethylamino)quinazolin-2-yl]amino}cyclohexyl)thiourea | | |
| 361 | N-(2,4-dichloro-6-methylphenyl)-N′-(cis-4-{[4- | 503 (M + H) | 1 |
| | (dimethylamino)quinazolin-2-yl]amino}cyclohexyl)thiourea | | |
| 362 | N-(cis-4-{[4-(dimethylamino)quinazolin-2-yl]amino}cyclohexyl)- | 449 (M + H) | 2 |
| | N′-(2,5-dimethylphenyl)thiourea | | |
| 363 | N-(2-bromo-4-isopropylphenyl)-N′-(cis-4-{[4- | 541 (M + H) | 2 |
| | (dimethylamino)quinazolin-2-yl]amino}cyclohexyl)thiourea | | |
| 364 | N-(2-bromo-5-fluorophenyl)-N′-(cis-4-{[4- | 517 (M + H) | 2 |
| | (dimethylamino)quinazolin-2-yl]amino}cyclohexyl)thiourea | | |
| 365 | N-(cis-4-{[4-(dimethylamino)quinazolin-2-yl]amino}cyclohexyl)- | 465 (M + H) | 1 |
| | N′-(2-ethoxyphenyl)thiourea | | |
| 366 | N-(cis-4-{[4-(dimethylamino)quinazolin-2-yl]amino}cyclohexyl)- | 477 (M + H) | 1 |
| | N′-(2-isopropyl-6-methylphenyl)thiourea | | |
| 367 | N-(cis-4-{[4-(dimethylamino)quinazolin-2-yl]amino}cyclohexyl)- | 465 (M + H) | 2 |
| | N′-(2-methoxybenzyl)thiourea | | |
| 368 | N-(2,3-dihydro-1,4-benzodioxin-6-yl)-N′-(cis-4-{[4- | 479 (M + H) | 1 |
| | (dimethylamino)quinazolin-2-yl]amino}cyclohexyl)thiourea | | |
| 369 | N-(cis-4-{[4-(dimethylamino)quinazolin-2-yl]amino}cyclohexyl)- | 449 (M + H) | 3 |
| | N′-(3,4-dimethylphenyl)thiourea | | |
| 370 | N-1,3-benzodioxol-5-yl-N′-(cis-4-{[4-(dimethylamino)quinazolin- | 465 (M + H) | 1 |
| | 2-yl]amino}cyclohexyl)thiourea | | |
| 371 | N-(3-chloro-2-methylphenyl)-N′-(cis-4-{[4- | 469 (M + H) | 1 |
| | (dimethylamino)quinazolin-2-yl]amino}cyclohexyl)thiourea | | |
| 372 | N-[4-bromo-2-(trifluoromethoxy)phenyl]-N′-(cis-4-{[4- | 583 (M + H) | 1 |
| | (dimethylamino)quinazolin-2-yl]amino}cyclohexyl)thiourea | | |
| 373 | N-(4-chloro-2,5-dimethoxyphenyl)-N′-(cis-4-{[4- | 515 (M + H) | 1 |
| | (dimethylamino)quinazolin-2-yl]amino}cyclohexyl)thiourea | | |
| 374 | N-(cis-4-{[4-(dimethylamino)quinazolin-2-yl]amino}cyclohexyl)- | 477 (M + H) | 2 |
| | N′-(4-phenylbutyl)thiourea | | |
| 375 | N-(4-tert-butylphenyl)-N′-(cis-4-{[4-(dimethylamino)quinazolin- | 477 (M + H) | 3 |
| | 2-yl]amino}cyclohexyl)thiourea | | |
| 376 | N-(5-chloro-2-fluorophenyl)-N′-(cis-4-{[4- | 473 (M + H) | 3 |
| | (dimethylamino)quinazolin-2-yl]amino}cyclohexyl)thiourea | | |
| 377 | N-bicyclo[2.2.1]hept-2-yl-N′-(cis-4-{[4- | 439 (M + H) | 1 |
| | (dimethylamino)quinazolin-2-yl]amino}cyclohexyl)thiourea | | |
| 378 | N-bicyclo[2.2.1]hept-5-en-2-yl-N′-(cis-4-{[4- | 437 (M + H) | 3 |
| | (dimethylamino)quinazolin-2-yl]amino}cyclohexyl)thiourea | | |
| 379 | N-(cyclopropylmethyl)-N′-(cis-4-{[4-(dimethylamino)quinazolin- | 399 (M + H) | 3 |
| | 2-yl]amino}cyclohexyl)thiourea | | |
| 380 | ethyl 2-({[(cis-4-{[4-(dimethylamino)quinazolin-2- | 553 (M + H) | 3 |
| | yl]amino}cyclohexyl)amino]carbonothioyl}amino)-4,5,6,7- | | |
| | tetrahydro-1-benzothiophene-3-carboxylate | | |
| 381 | methyl 3-({[(cis-4-{[4-(dimethylamino)quinazolin-2- | 499 (M + H) | 1 |
| | yl]amino}cyclohexyl)amino]carbonothioyl}amino)-4- | | |
| | methylthiophene-2-carboxylate | | |
| 382 | methyl 3-({[(cis-4-{[4-(dimethylamino)quinazolin-2- | 485 (M + H) | 1 |
| | yl]amino}cyclohexyl)amino]carbonothioyl}amino)thiophene-2- | | |
| | carboxylate | | |
| 383 | N-(2-bromo-4-fluorophenyl)-N′-(cis-4-{[4- | 517 (M + H) | 2 |
| | (dimethylamino)quinazolin-2-yl]amino}cyclohexyl)thiourea | | |
| 384 | N-(3-chloro-4-fluorophenyl)-N′-(cis-4-{[4- | 473 (M + H) | 3 |
| | (dimethylamino)quinazolin-2-yl]amino}cyclohexyl)thiourea | | |
| 385 | N-(4-butyl-2-methylphenyl)-N′-(cis-4-{[4- | 491 (M + H) | 1 |
| | (dimethylamino)quinazolin-2-yl]amino}cyclohexyl)thiourea | | |
| 386 | N-[4-(dimethylamino)phenyl]-N′-(cis-4-{[4- | 464 (M + H) | 3 |
| | (dimethylamino)quinazolin-2-yl]amino}cyclohexyl)thiourea | | |
| 387 | N-[3-(diethylamino)propyl]-N′-(cis-4-{[4- | 458 (M + H) | 3 |
| | (dimethylamino)quinazolin-2-yl]amino}cyclohexyl)thiourea | | |
| 388 | N-(cis-4-{[4-(dimethylamino)quinazolin-2-yl]amino}cyclohexyl)- | 458 (M + H) | 3 |
| | N′-(2-morpholin-4-ylethyl)thiourea | | |
| 389 | N-[4-(dimethylamino)-1-naphthyl]-N′-(cis-4-{[4- | 514 (M + H) | 1 |
| | (dimethylamino)quinazolin-2-yl]amino}cyclohexyl)thiourea | | |
| 390 | N-(cis-4-{[4-(dimethylamino)quinazolin-2-yl]amino}cyclohexyl)- | 422 (M + H) | 3 |
| | N′-pyridin-3-ylthiourea | | |
| 391 | N-(4-{(E)-[4-(dimethylamino)phenyl]diazenyl}phenyl)-N′-(cis-4- | 568 (M + H) | 3 |
| | {[4-(dimethylamino)quinazolin-2-yl]amino}cyclohexyl)thiourea | | |
| 392 | N-(cis-4-{[4-(dimethylamino)quinazolin-2-yl]amino}cyclohexyl)- | 472 (M + H) | 3 |
| | N′-(3-morpholin-4-ylpropyl)thiourea | | |
| 393 | N-[4-(diethylamino)phenyl]-N′-(cis-4-{[4- | 492 (M + H) | 3 |
| | (dimethylamino)quinazolin-2-yl]amino}cyclohexyl)thiourea | | |
| 394 | N-(cis-4-{[4-(dimethylamino)quinazolin-2-yl]amino}cyclohexyl)- | 525 (M + H) | 3 |
| | N′-{4-[(E)-phenyldiazenyl]phenyl}thiourea | | |
| 395 | N-(cis-4-{[4-(dimethylamino)quinazolin-2-yl]amino}cyclohexyl)- | 456 (M + H) | 3 |
| | N′-(2-piperidin-1-ylethyl)thiourea | | |
| 396 | N-(cis-4-{[4-(dimethylamino)quinazolin-2-yl]amino}cyclohexyl)- | 436 (M + H) | 3 |
| | N′-(pyridin-3-ylmethyl)thiourea | | |
| 397 | N-(cis-4-{[4-(dimethylamino)quinazolin-2-yl]amino}cyclohexyl)- | 487 (M + H) | 3 |
| | N′-[4-(1H-pyrazol-1-yl)phenyl]thiourea | | |
| 398 | N-2,1,3-benzothiadiazol-4-yl-N′-(cis-4-{[4- | 479 (M + H) | 3 |
| | (dimethylamino)quinazolin-2-yl]amino}cyclohexyl)thiourea | | |
| 399 | N-2,1,3-benzothiadiazol-5-yl-N′-(cis-4-{[4- | 479 (M + H) | 3 |
| | (dimethylamino)quinazolin-2-yl]amino}cyclohexyl)thiourea | | |
| 400 | N-(cis-4-{[4-(dimethylamino)quinazolin-2-yl]amino}cyclohexyl)- | 440 (M + H) | 3 |
| | N′-(3,5-dimethylisoxazol-4-yl)thiourea | | |
| 401 | N-(cis-4-{[4-(dimethylamino)quinazolin-2-yl]amino}cyclohexyl)- | 488 (M + H) | 3 |
| | N′-[4-(1,3-oxazol-5-yl)phenyl]thiourea | | |
| 402 | N-(cis-4-{[4-(dimethylamino)quinazolin-2-yl]amino}cyclohexyl)- | 502 (M + H) | 1 |
| | N′-(5-methyl-3-phenylisoxazol-4-yl)thiourea | | |
| 403 | N-(cis-4-{[4-(dimethylamino)quinazolin-2-yl]amino}cyclohexyl)- | 507 (M + H) | 3 |
| | N′-(6-morpholin-4-ylpyridin-3-yl)thiourea | | |
| 404 | N-(cis-4-{[4-(dimethylamino)quinazolin-2-yl]amino}cyclohexyl)- | 514 (M + H) | 3 |
| | N′-(6-phenoxypyridin-3-yl)thiourea | | |
| 405 | N-(3-acetylphenyl)-N′-[(cis-4-{[4-(dimethylamino)quinazolin-2- | 461 (M + H) | 3 |
| | yl]amino}cyclohexyl)methyl]urea | | |
| 406 | N-1-adamantyl-N′-[(cis-4-{[4-(dimethylamino)quinazolin-2- | 477 (M + H) | 3 |
| | yl]amino}cyclohexyl)methyl]urea | | |
| 407 | N-(4-acetylphenyl)-N′-[(cis-4-{[4-(dimethylamino)quinazolin-2- | 461 (M + H) | 3 |
| | yl]amino}cyclohexyl)methyl]urea | | |
| 408 | N-({[(cis-4-{[4-(dimethylamino)quinazolin-2- | 447 (M + H) | 3 |
| | yl]amino}cyclohexyl)methyl]amino}carbonyl)benzamide | | |
| 409 | N-[3,5-bis(trifluoromethyl)phenyl]-N′-[(cis-4-{[4- | 555 (M + H) | 3 |
| | (dimethylamino)quinazolin-2-yl]amino}cyclohexyl)methyl]urea | | |
| 410 | N-benzyl-N′-[(cis-4-{[4-(dimethylamino)quinazolin-2- | 433 (M + H) | 3 |
| | yl]amino}cyclohexyl)methyl]urea | | |
| 411 | N-(2-bromophenyl)-N′-[(cis-4-{[4-(dimethylamino)quinazolin-2- | 497 (M + H) | 2 |
| | yl]amino}cyclohexyl)methyl]urea | | |
| 412 | N-biphenyl-2-yl-N′-[(cis-4-{[4-(dimethylamino)quinazolin-2- | 495 (M + H) | 2 |
| | yl]amino}cyclohexyl)methyl]urea | | |
| 413 | N-(4-bromophenyl)-N′-[(cis-4-{[4-(dimethylamino)quinazolin-2- | 497 (M + H) | 3 |
| | yl]amino}cyclohexyl)methyl]urea | | |
| 414 | N-butyl-N′-[(cis-4-{[4-(dimethylamino)quinazolin-2- | 399 (M + H) | 2 |
| | yl]amino}cyclohexyl)methyl]urea | | |
| 415 | N-(3-chlorophenyl)-N′-[(cis-4-{[4-(dimethylamino)quinazolin-2- | 453 (M + H) | 2 |
| | yl]amino}cyclohexyl)methyl]urea | | |
| 416 | N-(4-chlorophenyl)-N′-[(cis-4-{[4-(dimethylamino)quinazolin-2- | 453 (M + H) | 3 |
| | yl]amino}cyclohexyl)methyl]urea | | |
| 417 | N-cyclohexyl-N′-[(cis-4-{[4-(dimethylamino)quinazolin-2- | 425 (M + H) | 2 |
| | yl]amino}cyclohexyl)methyl]urea | | |
| 418 | N-(3-cyanophenyl)-N′-[(cis-4-{[4-(dimethylamino)quinazolin-2- | 444 (M + H) | 2 |
| | yl]amino}cyclohexyl)methyl]urea | | |
| 419 | N-(2-chlorophenyl)-N′-[(cis-4-{[4-(dimethylamino)quinazolin-2- | 453 (M + H) | 1 |
| | yl]amino}cyclohexyl)methyl]urea | | |
| 420 | N-[(cis-4-{[4-(dimethylamino)quinazolin-2- | 447 (M + H) | 1 |
| | yl]amino}cyclohexyl)methyl]-N′-(2,6-dimethylphenyl)urea | | |
| 421 | N-(3,4-dichlorophenyl)-N′-[(cis-4-{[4- | 487 (M + H) | 2 |
| | (dimethylamino)quinazolin-2-yl]amino}cyclohexyl)methyl]urea | | |
| 422 | N-(2,4-difluorophenyl)-N′-[(cis-4-{[4-(dimethylamino)quinazolin- | 455 (M + H) | 1 |
| | 2-yl]amino}cyclohexyl)methyl]urea | | |
| 423 | N-(2,4-dichlorophenyl)-N′-[(cis-4-{[4- | 487 (M + H) | 2 |
| | (dimethylamino)quinazolin-2-yl]amino}cyclohexyl)methyl]urea | | |
| 424 | N-(3,5-dichlorophenyl)-N′-[(cis-4-{[4- | 487 (M + H) | 1 |
| | (dimethylamino)quinazolin-2-yl]amino}cyclohexyl)methyl]urea | | |
| 425 | N-(2,3-dichlorophenyl)-N′-[(cis-4-{[4- | 487 (M + H) | 1 |
| | (dimethylamino)quinazolin-2-yl]amino}cyclohexyl)methyl]urea | | |
| 426 | N-(2,6-difluorophenyl)-N′-[(cis-4-{[4-(dimethylamino)quinazolin- | 455 (M + H) | 2 |
| | 2-yl]amino}cyclohexyl)methyl]urea | | |
| 427 | N-(2,5-dichlorophenyl)-N′-[(cis-4-{[4- | 487 (M + H) | 3 |
| | (dimethylamino)quinazolin-2-yl]amino}cyclohexyl)methyl]urea | | |
| 428 | N-[(cis-4-{[4-(dimethylamino)quinazolin-2- | 447 (M + H) | 1 |
| | yl]amino}cyclohexyl)methyl]-N′-(2,3-dimethylphenyl)urea | | |
| 429 | ethyl N-({[(cis-4-{[4-(dimethylamino)quinazolin-2- | 429 (M + H) | 3 |
| | yl]amino}cyclohexyl)methyl]amino}carbonyl)glycinate | | |
| 430 | ethyl 3-[({[(cis-4-{[4-(dimethylamino)quinazolin-2- | 491 (M + H) | 3 |
| | yl]amino}cyclohexyl)methyl]amino}carbonyl)amino]benzoate | | |
| 431 | ethyl 4-[({[(cis-4-{[4-(dimethylamino)quinazolin-2- | 491 (M + H) | 3 |
| | yl]amino}cyclohexyl)methyl]amino}carbonyl)amino]benzoate | | |
| 432 | N-[(cis-4-{[4-(dimethylamino)quinazolin-2- | 447 (M + H) | 2 |
| | yl]amino}cyclohexyl)methyl]-N′-(4-ethylphenyl)urea | | |
| 433 | N-[(cis-4-{[4-(dimethylamino)quinazolin-2- | 371 (M + H) | 3 |
| | yl]amino}cyclohexyl)methyl]-N′-ethylurea | | |
| 434 | N-[(cis-4-{[4-(dimethylamino)quinazolin-2- | 461 (M + H) | 1 |
| | yl]amino}cyclohexyl)methyl]-N′-(2-ethyl-6-methylphenyl)urea | | |
| 435 | ethyl N-({[(cis-4-{[4-(dimethylamino)quinazolin-2- | 485 (M + H) | 1 |
| | yl]amino}cyclohexyl)methyl]amino}carbonyl)leucinate | | |
| 436 | N-[(cis-4-{[4-(dimethylamino)quinazolin-2- | 482 (M + H) | 3 |
| | yl]amino}cyclohexyl)methyl]-N′-(4-fluoro-3-nitrophenyl)urea | | |
| 437 | N-[(cis-4-{[4-(dimethylamino)quinazolin-2- | 437 (M + H) | 1 |
| | yl]amino}cyclohexyl)methyl]-N′-(4-fluorophenyl)urea | | |
| 438 | N-[(cis-4-{[4-(dimethylamino)quinazolin-2- | 437 (M + H) | 2 |
| | yl]amino}cyclohexyl)methyl]-N′-(3-fluorophenyl)urea | | |
| 439 | N-[(cis-4-{[4-(dimethylamino)quinazolin-2- | 437 (M + H) | 2 |
| | yl]amino}cyclohexyl)methyl]-N′-(2-fluorophenyl)urea | | |
| 440 | N-[(cis-4-{[4-(dimethylamino)quinazolin-2- | 461 (M + H) | 3 |
| | yl]amino}cyclohexyl)methyl]-N′-(4-isopropylphenyl)urea | | |
| 441 | N-[(cis-4-{[4-(dimethylamino)quinazolin-2-yl]amino}cyclohexylmethyl]- | 501 (M + H) | 2 |
| | N′-[1-(3-isopropenylphenyl)-1-methylethyl]urea | | |
| 442 | methyl N-({[(cis-4-{[4-(dimethylamino)quinazolin-2- | 489 (M + H) | 2 |
| | yl]amino}cyclohexyl)methyl]amino}carbonyl)methioninate | | |
| 443 | N-[(cis-4-{[4-(dimethylamino)quinazolin-2- | 385 (M + H) | 3 |
| | yl]amino}cyclohexyl)methyl]-N′-isopropylurea | | |
| 444 | N-[(cis-4-{[4-(dimethylamino)quinazolin-2- | 449 (M + H) | 2 |
| | yl]amino}cyclohexyl)methyl]-N′-(4-methoxyphenyl)urea | | |
| 445 | N-[(cis-4-{[4-(dimethylamino)quinazolin-2- | 478 (M + H) | 2 |
| | yl]amino}cyclohexyl)methyl]-N′-(4-methyl-2-nitrophenyl)urea | | |
| 446 | N-[(cis-4-{[4-(dimethylamino)quinazolin-2- | 449 (M + H) | 2 |
| | yl]amino}cyclohexyl)methyl]-N′-(2-methoxyphenyl)urea | | |
| 447 | N-[(cis-4-{[4-(dimethylamino)quinazolin-2- | 449 (M + H) | 2 |
| | yl]amino}cyclohexyl)methyl]-N′-(3-methoxyphenyl)urea | | |
| 448 | N-[(cis-4-{[4-(dimethylamino)quinazolin-2- | 465 (M + H) | 1 |
| | yl]amino}cyclohexyl)methyl]-N′-[4-(methylthio)phenyl]urea | | |
| 449 | N-[(cis-4-{[4-(dimethylamino)quinazolin-2- | 463 (M + H) | 3 |
| | yl]amino}cyclohexyl)methyl]-N′-(4-methoxybenzyl)urea | | |
| 450 | N-[(cis-4-{[4-(dimethylamino)quinazolin-2- | 469 (M + H) | 2 |
| | yl]amino}cyclohexyl)methyl]-N′-1-naphthylurea | | |
| 451 | N-[(cis-4-{[4-(dimethylamino)quinazolin-2- | 459 (M + H) | 3 |
| | yl]amino}cyclohexyl)methyl]-N′-[(2S)-2-phenylcyclopropyl]urea | | |
| 452 | N-[(cis-4-{[4-(dimethylamino)quinazolin-2- | 419 (M + H) | 1 |
| | yl]amino}cyclohexyl)methyl]-N′-phenylurea | | |
| 453 | N-[(cis-4-{[4-(dimethylamino)quinazolin-2- | 511 (M + H) | 3 |
| | yl]amino}cyclohexyl)methyl]-N′-(4-phenoxyphenyl)urea | | |
| 454 | N-[(cis-4-{[4-(dimethylamino)quinazolin-2- | 413 (M + H) | 2 |
| | yl]amino}cyclohexyl)methyl]-N′-pentylurea | | |
| 455 | N-[(cis-4-{[4-(dimethylamino)quinazolin-2- | 487 (M + H) | 1 |
| | yl]amino}cyclohexyl)methyl]-N′-[2-(trifluoromethyl)phenyl]urea | | |
| 456 | N-[(cis-4-{[4-(dimethylamino)quinazolin-2- | 487 (M + H) | 3 |
| | yl]amino}cyclohexyl)methyl]-N′-[3-(trifluoromethyl)phenyl]urea | | |
| 457 | N-[(cis-4-{[4-(dimethylamino)quinazolin-2- | 433 (M + H) | 1 |
| | yl]amino}cyclohexyl)methyl]-N′-(4-methylphenyl)urea | | |
| 458 | N-[(cis-4-{[4-(dimethylamino)quinazolin-2- | 461 (M + H) | 1 |
| | yl]amino}cyclohexyl)methyl]-N′-mesitylurea | | |
| 459 | N-[(cis-4-{[4-(dimethylamino)quinazolin-2- | 433 (M + H) | 2 |
| | yl]amino}cyclohexyl)methyl]-N′-(3-methylphenyl)urea | | |
| 460 | N-[(cis-4-{[4-(dimethylamino)quinazolin-2- | 433 (M + H) | 1 |
| | yl]amino}cyclohexyl)methyl]-N′-(2-methylphenyl)urea | | |
| 461 | N-[(cis-4-{[4-(dimethylamino)quinazolin-2- | 497 (M + H) | 3 |
| | yl]amino}cyclohexyl)methyl]-N′-[1-(1-naphthyl)ethyl]urea | | |
| 462 | methyl N-({[(cis-4-{[4-(dimethylamino)quinazolin-2- | 505 (M + H) | 3 |
| | yl]amino}cyclohexyl)methyl]amino}carbonyl)phenylalaninate | | |
| 463 | N-[(cis-4-{[4-(dimethylamino)quinazolin-2- | 521 (M + H) | 1 |
| | yl]amino}cyclohexyl)methyl]-N′-(2,4,6-trichlorophenyl)urea | | |
| 464 | N-(3-chloro-4-methylphenyl)-N′-[(cis-4-{[4- | 467 (M + H) | 3 |
| | (dimethylamino)quinazolin-2-yl]amino}cyclohexyl)methyl]urea | | |
| 465 | N-[(cis-4-{[4-(dimethylamino)quinazolin-2- | 447 (M + H) | 2 |
| | yl]amino}cyclohexyl)methyl]-N′-(1-phenylethyl)urea | | |
| 466 | 1-[4-(4-Dimethylamino-quinazolin-2-ylamino)- | 447 (M + H) | 2 |
| | cyclohexylmethyl]-3-(1-phenyl-ethyl)-urea | | |
| 467 | 1-[4-(4-Dimethylamino-quinazolin-2-ylamino)- | 497 (M + H) | 2 |
| | cyclohexylmethyl]-3-(1-naphthalen-1-yl-ethyl)-urea | | |
| 468 | N-(2,6-diisopropylphenyl)-N′-[(cis-4-{[4- | 503 (M + H) | 1 |
| | (dimethylamino)quinazolin-2-yl]amino}cyclohexyl)methyl]urea | | |
| 469 | N-[2-(difluoromethoxy)phenyl]-N′-[(cis-4-{[4- | 485 (M + H) | 2 |
| | (dimethylamino)quinazolin-2-yl]amino}cyclohexyl)methyl]urea | | |
| 470 | methyl 2-[({[(cis-4-{[4-(dimethylamino)quinazoalin-2- | 477 (M + H) | 3 |
| | yl]amino}cyclohexyl)methyl]amino}carbonyl)amino]benzoate | | |
| 471 | N-[(cis-4-{[4-(dimethylamino)quinazolin-2- | 465 (M + H) | 2 |
| | yl]amino}cyclohexyl)methyl]-N′-[2-(methylthio)phenyl]urea | | |
| 472 | N-[(cis-4-{[4-(dimethylamino)quinazolin-2- | 555 (M + H) | 2 |
| | yl]amino}cyclohexyl)methyl]-N′-(2,3,5,6-tetrachlorophenyl)urea | | |
| 473 | N-[(cis-4-{[4-(dimethylamino)quinazolin-2-yl]amino}- | 492 (M + H) | 1 |
| | cyclohexyl)-methyl]-N′-(2,3-dimethyl-6-nitrophenyl)urea | | |
| 474 | N-[(cis-4-{[4-(dimethylamino)quinazolin-2- | 521 (M + H) | 3 |
| | yl]amino}cyclohexyl)methyl]-N′-(2,4,5-trichlorophenyl)urea | | |
| 475 | N-[(cis-4-{[4-(dimethylamino)quinazolin-2- | 652 (M + H) | 1 |
| | yl]amino}cyclohexyl)methyl]-N′-(2,4,6-tribromophenyl)urea | | |
| 476 | N-(2,4-dibromo-6-fluorophenyl)-N′-[(cis-4-{[4- | 593 (M + H) | 1 |
| | (dimethylamino)quinazolin-2-yl]amino}cyclohexyl)methyl]urea | | |
| 477 | N-(2,4-dibromophenyl)-N′-[(cis-4-{[4- | 575 (M + H) | 3 |
| | (dimethylamino)quinazolin-2-yl]amino}cyclohexyl)methyl]urea | | |
| 478 | N-(2,4-dichlorobenzyl)-N′-[(cis-4-{[4- | 501 (M + H) | 2 |
| | (dimethylamino)quinazolin-2-yl]amino}cyclohexyl)methyl]urea | | |
| 479 | N-(2,4-dimethoxyphenyl)-N′-[(cis-4-{[4- | 479 (M + H) | 3 |
| | (dimethylamino)quinazolin-2-yl]amino}cyclohexyl)methyl]urea | | |
| 480 | N-(2,5-dimethylphenyl)-N′-[(cis-4-{[4- | 479 (M + H) | 2 |
| | (dimethylamino)quinazolin-2-yl]amino}cyclohexyl)methyl]urea | | |
| 481 | N-[(cis-4-{[4-(dimethylamino)quinazolin-2- | 447 (M + H) | 3 |
| | yl]amino}cyclohexyl)methyl]-N′-(2,5-dimethylphenyl)urea | | |
| 482 | N-(2,6-dibromo-4-fluorophenyl)-N′-[(cis-4-{[4- | 593 (M + H) | 1 |
| | (dimethylamino)quinazolin-2-yl]amino}cyclohexyl)methyl]urea | | |
| 483 | N-(2,6-dichlorophenyl)-N′-[(cis-4-{[4- | 487 (M + H) | 1 |
| | (dimethylamino)quinazolin-2-yl]amino}cyclohexyl)methyl]urea | | |
| 484 | N-(2,6-diethylphenyl)-N′-[(cis-4-{[4-(dimethylamino)quinazolin- | 475 (M + H) | 1 |
| | 2-yl]amino}cyclohexyl)methyl]urea | | |
| 485 | N-(2-benzylphenyl)-N′-[(cis-4-{[4-(dimethylamino)quinazolin-2- | 509 (M + H) | 3 |
| | yl]amino}cyclohexyl)methyl]urea | | |
| 486 | N-(2-chloro-5-methylphenyl)-N′-[(cis-4-{[4- | 467 (M + H) | 2 |
| | (dimethylamino)quinazolin-2-yl]amino}cyclohexyl)methyl]urea | | |
| 487 | N-(2-chloro-5-nitrophenyl)-N′-[(cis-4-{[4- | 498 (M + H) | 3 |
| | (dimethylamino)quinazolin-2-yl]amino}cyclohexyl)methyl]urea | | |
| 488 | N-[2-chloro-6-(trifluoromethyl)phenyl]-N′-[(cis-4-{[4- | 521 (M + H) | 1 |
| | (dimethylamino)quinazolin-2-yl]amino}cyclohexyl)methyl]urea | | |
| 489 | N-(2-chloro-6-methylphenyl)-N′-[(cis-4-{[4- | 467 (M + H) | 1 |
| | (dimethylamino)quinazolin-2-yl]amino}cyclohexyl)methyl]urea | | |
| 490 | N-(2-chlorobenzyl)-N′-[(cis-4-{[4-(dimethylamino)quinazolin-2- | 467 (M + H) | 1 |
| | yl]amino}cyclohexyl)methyl]urea | | |
| 491 | ethyl 2-[({[(cis-4-{[4-(dimethylamino)quinazolin-2- | 491 (M + H) | 3 |
| | yl]amino}cyclohexyl)methyl]amino}carbonyl)amino]benzoate | | |
| 492 | N-[(cis-4-{[4-(dimethylamino)quinazolin-2- | 463 (M + H) | ? |
| | yl]amino}cyclohexyl)methyl]-N′-(2-ethoxyphenyl)urea | | |
| 493 | N-[(cis-4-{[4-(dimethylamino)quinazolin-2- | 489 (M + H) | 1 |
| | yl]amino}cyclohexyl)methyl]-N′-(2-ethyl-6-isopropylphenyl)urea | | |
| 494 | N-[(cis-4-{[4-(dimethylamino)quinazolin-2- | 447 (M + H) | 1 |
| | yl]amino}cyclohexyl)methyl]-N′-(2-ethylphenyl)urea | | |
| 495 | N-[(cis-4-{[4-(dimethylamino)quinazolin-2-yl]amino}- | 505 (M + H) | 3 |
| | cyclohexyl)methyl]-N′-[2-fluoro-3-(trifluoromethyl)phenyl]urea | | |
| 496 | N-[(cis-4-{[4-(dimethylamino)quinazolin-2-yl]amino}- | 505 (M + H) | 3 |
| | cyclohexyl)methyl]-N′-[2-fluoro-5-(trifluoromethyl)phenyl]urea | | |
| 497 | N-[(cis-4-{[4-(dimethylamino)quinazolin-2- | 451 (M + H) | 3 |
| | yl]amino}cyclohexyl)methyl]-N′-(2-fluoro-5-methylphenyl)urea | | |
| 498 | N-[(cis-4-{[4-(dimethylamino)quinazolin-2- | 482 (M + H) | 2 |
| | yl]amino}cyclohexyl)methyl]-N′-(2-fluoro-5-nitrophenyl)urea | | |
| 499 | N-[(cis-4-{[4-(dimethylamino)quinazolin-2- | 451 (M + H) | 2 |
| | yl]amino}cyclohexyl)methyl]-N′-(2-fluorobenzyl)urea | | |
| 500 | N-[(cis-4-{[4-(dimethylamino)quinazolin-2- | 545 (M + H) | 1 |
| | yl]amino}cyclohexyl)methyl]-N′-(2-iodophenyl)urea | | |
| 501 | N-[(cis-4-{[4-(dimethylamino)quinazolin-2-yl]amino}- | 475 (M + H) | 1 |
| | cyclohexyl)methyl]-N′-(2-isopropyl-6-methylphenyl)urea | | |
| 502 | N-[(cis-4-{[4-(dimethylamino)quinazolin-2- | 461 (M + H) | 1 |
| | yl]amino}cyclohexyl)methyl]-N′-(2-isopropylphenyl)urea | | |
| 503 | N-[(cis-4-{[4-(dimethylamino)quinazolin-2- | 494 (M + H) | 3 |
| | yl]amino}cyclohexyl)methyl]-N′-(2-methoxy-4-nitrophenyl)urea | | |
| 504 | N-[(cis-4-{[4-(dimethylamino)quinazolin-2-yl]amino}- | 463 (M + H) | 1 |
| | cyclohexyl)methyl]-N′-(2-methoxy-5-methylphenyl)urea | | |
| 505 | N-[(cis-4-{[4-(dimethylamino)quinazolin-2- | 494 (M + H) | 2 |
| | yl]amino}cyclohexyl)methyl]-N′-(2-methoxy-5-nitrophenyl)urea | | |
| 506 | N-[(cis-4-{[4-(dimethylamino)quinazolin-2- | 478 (M + H) | 1 |
| | yl]amino}cyclohexyl)methyl]-N′-(2-methyl-3-nitrophenyl)urea | | |
| 507 | N-[(cis-4-{[4-(dimethylamino)quinazolin-2- | 478 (M + H) | 2 |
| | yl]amino}cyclohexyl)methyl]-N′-(2-methoxy-4-nitrophenyl)urea | | |
| 508 | N-[(cis-4-{[4-(dimethylamino)quinazolin-2- | 478 (M + H) | 2 |
| | yl]amino}cyclohexyl)methyl]-N′-(2-methoxy-5-nitrophenyl)urea | | |
| 509 | N-[(cis-4-{[4-(dimethylamino)quinazolin-2- | 478 (M + H) | 1 |
| | yl]amino}cyclohexyl)methyl]-N′-(2-methyl-6-nitrophenyl)urea | | |
| 510 | N-[(cis-4-{[4-(dimethylamino)quinazolin-2- | 447 (M + H) | 2 |
| | yl]amino}cyclohexyl)methyl]-N′-(2-methylbenzyl)urea | | |
| 511 | N-[(cis-4-{[4-(dimethylamino)quinazolin-2- | 469 (M + H) | 3 |
| | yl]amino}cyclohexyl)methyl]-N′-2-naphthylurea | | |
| 512 | N-[(cis-4-{[4-(dimethylamino)quinazolin-2- | 464 (M + H) | 2 |
| | yl]amino}cyclohexyl)methyl]-N′-(2-nitrophenyl)urea | | |
| 513 | N-[(cis-4-{[4-(dimethylamino)quinazolin-2- | 461 (M + H) | 1 |
| | yl]amino}cyclohexyl)methyl]-N′-(2-propylphenyl)urea | | |
| 514 | N-[(cis-4-{[4-(dimethylamino)quinazolin-2- | 511 (M + H) | 2 |
| | yl]amino}cyclohexyl)methyl]-N′-(2-phenoxyphenyl)urea | | |
| 515 | N-(2-tert-butyl-6-methylphenyl)-N′-[(cis-4-{[4- | 489 (M + H) | 1 |
| | (dimethylamino)quinazolin-2-yl]amino}cyclohexyl)methyl]urea | | |
| 516 | N-(2-tert-butylphenyl)-N′-[(cis-4-{[4-(dimethylamino)quinazolin- | 475 (M + H) | 1 |
| | 2-yl]amino}cyclohexyl)methyl]urea | | |
| 517 | N-[(cis-4-{[4-(dimethylamino)quinazolin-2- | 465 (M + H) | 2 |
| | yl]amino}cyclohexyl)methyl]-N′-[3-(methylthio)phenyl]urea | | |
| 518 | N-[(cis-4-{[4-(dimethylamino)quinazolin-2-yl]amino}- | 519 (M + H) | 3 |
| | cyclohexyl)methyl]-N′-{3-[(trifluoromethyl)thio]phenyl}urea | | |
| 519 | N-1,3-benzodioxol-5-yl-N′-[(cis-4-{[4- | 463 (M + H) | 3 |
| | (dimethylamino)quinazolin-2-yl]amino}cyclohexyl)methyl]urea | | |
| 520 | N-[(cis-4-{[4-(dimethylamino)quinazolin-2- | 509 (M + H) | 3 |
| | yl]amino}cyclohexyl)methyl]-N′-(3,4,5-trimethoxyphenyl)urea | | |
| 521 | N-(3,4-dichlorobenzyl)-N′-[(cis-4-{[4- | 501 (M + H) | 3 |
| | (dimethylamino)quinazolin-2-yl]amino}cyclohexyl)methyl]urea | | |
| 522 | N-(3,4-difluorophenyl)-N′-[(cis-4-{[4-(dimethylamino)quinazolin- | 455 (M + H) | 1 |
| | 2-yl]amino}cyclohexyl)methyl]urea | | |
| 523 | N-(3,4-dimethoxyphenyl)-N′-[(cis-4-{[4- | 479 (M + H) | 3 |
| | (dimethylamino)quinazolin-2-yl]amino}cyclohexyl)methyl]urea | | |
| 524 | N-(3,5-difluorobenzyl)-N′-[(cis-4-{[4-(dimethylamino)quinazolin- | 455 (M + H) | 1 |
| | 2-yl]amino}cyclohexyl)methyl]urea | | |
| 525 | N-(3,5-dimethoxyphenyl)-N′-[(cis-4-{[4- | 479 (M + H) | 2 |
| | (dimethylamino)quinazolin-2-yl]amino}cyclohexyl)methyl]urea | | |
| 526 | N-[(cis-4-{[4-(dimethylamino)quinazolin-2- | 447 (M + H) | 3 |
| | yl]amino}cyclohexyl)methyl]-N′-(3,5-dimethylphenyl)urea | | |
| 527 | methyl 3-[({[(cis-4-{[4-(dimethylamino)quinazolin-2- | 477 (M + H) | 3 |
| | yl]amino}cyclohexyl)methyl]amino}carbonyl)amino]benzoate | | |
| 528 | N-(3-chloro-2-methylphenyl)-N′-[(cis-4-{[4- | 467 (M + H) | 1 |
| | (dimethylamino)quinazolin-2-yl]amino}cyclohexyl)methyl]urea | | |
| 529 | N-(3-chloro-4-fluorophenyl)-N′-[(cis-4-{[4- | 471 (M + H) | 1 |
| | (dimethylamino)quinazolin-2-yl]amino}cyclohexyl)methyl]urea | | |
| 530 | N-(3-chloro-4-methoxyphenyl)-N′-[(cis-4-{[4- | 483 (M + H) | 3 |
| | (dimethylamino)quinazolin-2-yl]amino}cyclohexyl)methyl]urea | | |
| 531 | N-[(cis-4-{[4-(dimethylamino)quinazolin-2- | 447 (M + H) | 2 |
| | yl]amino}cyclohexyl)methyl]-N′-(3-ethylphenyl)urea | | |
| 532 | N-[(cis-4-{[4-(dimethylamino)quinazolin-2-yl]amino}- | 505 (M + H) | 2 |
| | cyclohexyl)methyl]-N′-[3-fluoro-5-(trifluoromethyl)phenyl]urea | | |
| 533 | N-[(cis-4-{[4-(dimethylamino)quinazolin-2- | 451 (M + H) | 2 |
| | yl]amino}cyclohexyl)methyl]-N′-(3-fluorobenzyl)urea | | |
| 534 | N-[(cis-4-{[4-(dimethylamino)quinazolin-2- | 511 (M + H) | 3 |
| | yl]amino}cyclohexyl)methyl]-N′-(3-phenoxyphenyl)urea | | |
| 535 | butyl 4-[({[(cis-4-{[4-(dimethylamino)quinazolin-2- | 519 (M + H) | 3 |
| | yl]amino}cyclohexyl)methyl]amino}carbonyl)amino]benzoate | | |
| 536 | N-[(cis-4-{[4-(dimethylamino)quinazolin-2- | 487 (M + H) | 3 |
| | yl]amino}cyclohexyl)methyl]-N′-[4-(trifluoromethyl)phenyl]urea | | |
| 537 | N-[(cis-4-{[4-(dimethylamino)quinazolin-2-yl]amino}- | 519 (M + H) | 3 |
| | cyclohexyl)methyl]-N′-{4-[(trifluoromethyl)thio]phenyl}urea | | |
| 538 | N-[(cis-4-{[4-(dimethylamino)quinazolin-2-yl]amino}- | 492 (M + H) | 2 |
| | cyclohexyl)methyl]-N′-(4,5-dimethyl-2-nitrophenyl)urea | | |
| 539 | N-[4-(benzyloxy)phenyl]-N′-[(cis-4-{[4- | 525 (M + H) | 3 |
| | (dimethylamino)quinazolin-2-yl]amino}cyclohexyl)methyl]urea | | |
| 540 | N-(4-benzylphenyl)-N′-[(cis-4-{[4-(dimethylamino)quinazolin-2- | 509 (M + H) | 3 |
| | yl]amino}cyclohexyl)methyl]urea | | |
| 541 | N-[4-bromo-2-(trifluoromethyl)phenyl]-N′-[(cis-4-{[4- | 565 (M + H) | 2 |
| | (dimethylamino)quinazolin-2-yl]amino}cyclohexyl)methyl]urea | | |
| 542 | N-(4-bromo-2,6-difluorophenyl)-N′-[(cis-4-{[4- | 533 (M + H) | 1 |
| | (dimethylamino)quinazolin-2-yl]amino}cyclohexyl)methyl]urea | | |
| 543 | N-(4-bromo-2-chlorophenyl)-N′-[(cis-4-{[4- | 531 (M + H) | 3 |
| | (dimethylamino)quinazolin-2-yl]amino}cyclohexyl)methyl]urea | | |
| 544 | N-(4-bromobenzyl)-N′-[(cis-4-{[4-(dimethylamino)quinazolin-2- | 511 (M + H) | 3 |
| | yl]amino}cyclohexyl)methyl]urea | | |
| 545 | N-[4-chloro-2-(trifluoromethyl)phenyl]-N′-[(cis-4-{[4- | 521 (M + H) | 1 |
| | (dimethylamino)quinazolin-2-yl]amino}cyclohexyl)methyl]urea | | |
| 546 | N-(4-chloro-2-methylphenyl)-N′-[(cis-4-{[4- | 467 (M + H) | 2 |
| | (dimethylamino)quinazolin-2-yl]amino}cyclohexyl)methyl]urea | | |
| 547 | N-(4-chloro-2-nitrophenyl)-N′-[(cis-4-{[4- | 498 (M + H) | 3 |
| | (dimethylamino)quinazolin-2-yl]amino}cyclohexyl)methyl]urea | | |
| 548 | N-[4-chloro-3-(trifluoromethyl)phenyl]-N′-[(cis-4-{[4- | 521 (M + H) | 3 |
| | (dimethylamino)quinazolin-2-yl]amino}cyclohexyl)methyl]urea | | |
| 549 | N-(4-cyanophenyl)-N′-[(cis-4-{[4-(dimethylamino)quinazolin-2- | 444 (M + H) | 1 |
| | yl]amino}cyclohexyl)methyl]urea | | |
| 550 | N-[(cis-4-{[4-(dimethylamino)quinazolin-2- | 463 (M + H) | 3 |
| | yl]amino}cyclohexyl)methyl]-N′-(4-ethoxyphenyl)urea | | |
| 551 | N-[(cis-4-{[4-(dimethylamino)quinazolin-2- | 482 (M + H) | 2 |
| | yl]amino}cyclohexyl)methyl]-N′-(4-fluoro-2-nitrophenyl)urea | | |
| 552 | N-[(cis-4-{[4-(dimethylamino)quinazolin-2-yl]amino}- | 505 (M + H) | 3 |
| | cyclohexyl)methyl]-N′-[4-fluoro-3-(trifluoromethyl)phenyl]urea | | |
| 553 | N-[(cis-4-{[4-(dimethylamino)quinazolin-2- | 451 (M + H) | 2 |
| | yl]amino}cyclohexyl)methyl]-N′-(4-fluorobenzyl)urea | | |
| 554 | N-[(cis-4-{[4-(dimethylamino)quinazolin-2- | 533 (M + H) | 3 |
| | yl]amino}cyclohexyl)methyl]-N′-[4-(heptyloxy)phenyl]urea | | |
| 555 | N-[(cis-4-{[4-(dimethylamino)quinazolin-2- | 545 (M + H) | 2 |
| | yl]amino}cyclohexyl)methyl]-N′-(4-iodophenyl)urea | | |
| 556 | N-[(cis-4-{[4-(dimethylamino)quinazolin-2-yl]amino}- | 463 (M + H) | 2 |
| | cyclohexyl)methyl]-N′-(4-methoxy-2-methylphenyl)urea | | |
| 557 | N-[(cis-4-{[4-(dimethylamino)quinazolin-2- | 494 (M + H) | 3 |
| | yl]amino}cyclohexyl)methyl]-N′-(4-methoxy-2-nitrophenyl)urea | | |
| 558 | N-[(cis-4-{[4-(dimethylamino)quinazolin-2- | 478 (M + H) | 2 |
| | yl]amino}cyclohexyl)methyl]-N′-(4-methyl-3-nitrophenyl)urea | | |
| 559 | N-[(cis-4-{[4-(dimethylamino)quinazolin-2- | 447 (M + H) | 3 |
| | yl]amino}cyclohexyl)methyl]-N′-(4-methylbenzyl)urea | | |
| 560 | N-(4-butoxyphenyl)-N′-[(cis-4-{[4-(dimethylamino)quinazolin-2- | 491 (M + H) | 3 |
| | yl]amino}cyclohexyl)methyl]urea | | |
| 561 | N-(4-butylphenyl)-N′-[(cis-4-{[4-(dimethylamino)quinazolin-2- | 475 (M + H) | 3 |
| | yl]amino}cyclohexyl)methyl]urea | | |
| 562 | N-biphenyl-4-yl-N′-[(cis-4-{[4-(dimethylamino)quinazolin-2- | 495 (M + H) | 3 |
| | yl]amino}cyclohexyl)methyl]urea | | |
| 563 | N-(5-chloro-2,4-dimethoxyphenyl)-N′-[(cis-4-{[4- | 513 (M + H) | 3 |
| | (dimethylamino)quinazolin-2-yl]amino}cyclohexyl)methyl]urea | | |
| 564 | N-(5-chloro-2-methoxyphenyl)-N′-[(cis-4-{[4- | 483 (M + H) | 3 |
| | (dimethylamino)quinazolin-2-yl]amino}cyclohexyl)methyl]urea | | |
| 565 | N-(5-chloro-2-methylphenyl)-N′-[(cis-4-{[4- | 467 (M + H) | 2 |
| | (dimethylamino)quinazolin-3-yl]amino}cyclohexyl)methyl]urea | | |
| 566 | N-(5-chloro-2-nitrophenyl)-N′-[(cis-4-{[4- | 498 (M + H) | 3 |
| | (dimethylamino)quinazolin-2-yl]amino}cyclohexyl)methyl]urea | | |
| 567 | N-[(cis-4-{[4-(dimethylamino)quinazolin-2- | 451 (M + H) | 2 |
| | yl]amino}cyclohexyl)methyl]-N′-(5-fluoro-2-methylphenyl)urea | | |
| 568 | N-(2,3-dihydro-1H-inden-5-yl)-N′-[(cis-4-{[4- | 459 (M + H) | 3 |
| | (dimethylamino)quinazolin-2-yl]amino}cyclohexyl)methyl]urea | | |
| 569 | N-[(cis-4-{[4-(dimethylamino)quinazolin-2- | 507 (M + H) | 3 |
| | yl]amino}cyclohexyl)methyl]-N′-9H-fluoren-2-ylurea | | |
| 570 | N-[(cis-4-{[4-(dimethylamino)quinazolin-2- | 507 (M + H) | 3 |
| | yl]amino}cyclohexyl)methyl]-N′-9H-fluoren-9-ylurea | | |
| 571 | N-[(cis-4-{[4-(dimethylamino)quinazolin-2- | 447 (M + H) | 3 |
| | yl]amino}cyclohexyl)methyl]-N′-(2-phenylethyl)urea | | |
| 572 | N-cyclopentyl-N′-[(cis-4-{[4-(dimethylamino)quinazolin-2- | 411 (M + H) | 2 |
| | yl]amino}cyclohexyl)methyl]urea | | |
| 573 | N-[(cis-4-{[4-(dimethylamino)quinazolin-2- | 509 (M + H) | 1 |
| | yl]amino}cyclohexyl)methyl]-N′-(diphenylmethyl)urea | | |
| 574 | methyl 4-[({[(cis-4-{[4-(dimethylamino)quinazolin-2- | 477 (M + H) | 3 |
| | yl]amino}cyclohexyl)methyl]amino}carbonyl)amino]benzoate | | |
| 575 | N-[1-(4-bromophenyl)ethyl]-N′-[(cis-4-{[4- | 525 (M + H) | 3 |
| | (dimethylamino)quinazolin-2-yl]amino}cyclohexyl)methyl]urea | | |
| 576 | N-[(cis-4-{[4-(dimethylamino)quinazolin-2-yl]amino}- | 503 (M + H) | 3 |
| | cyclohexyl)methyl]-N′-[2-(trifluoromethoxy)phenyl]urea | | |
| 577 | N-(3-acetylphenyl)-N′-[(cis-4-{[4-(dimethylamino)quinazolin-2- | 477 (M + H) | 3 |
| | yl]amino}cyclohexyl)methyl]thiourea | | |
| 578 | N-(4-acetylphenyl)-N′-[(cis-4-{[4-(dimethylamino)quinazolin-2- | 477 (M + H) | 3 |
| | yl]amino}cyclohexyl)methyl]thiourea | | |
| 579 | N-[3,5-bis(trifluoromethyl)phenyl]-N′-[(cis-4-{[4-(dimethylamino) | 571 (M + H) | 3 |
| | quinazolin-2-yl]amino}cyclohexyl)methyl]thiourea | | |
| 580 | N-benzyl-N′-[(cis-4-{[4-(dimethylamino)quinazolin-2- | 449 (M + H) | 3 |
| | yl]amino}cyclohexyl)methyl]thiourea | | |
| 581 | N-(3-bromophenyl)-N′-[(cis-4-{[4-(dimethylamino)quinazolin-2- | 513 (M + H) | 3 |
| | yl]amino}cyclohexyl)methyl]thiourea | | |
| 582 | N-(4-bromophenyl)-N′-[(cis-4-{[4-(dimethylamino)quinazolin-2- | 513 (M + H) | 3 |
| | yl]amino}cyclohexyl)methyl]thiourea | | |
| 583 | N-butyl-N′-[(cis-4-{[4-(dimethylamino)quinazolin-2- | 415 (M + H) | 3 |
| | yl]amino}cyclohexyl)methyl]thiourea | | |
| 584 | N-(4-cyanophenyl)-N′-[(cis-4-{[4-(dimethylamino)quinazolin-2- | 460 (M + H) | 3 |
| | yl]amino}cyclohexyl)methyl]thiourea | | |
| 585 | N-cyclohexyl-N′-[(cis-4-{[4-(dimethylamino)quinazolin-2- | 441 (M + H) | 3 |
| | yl]amino}cyclohexyl)methyl]thiourea | | |
| 586 | N-cyclopentyl-N′-[(cis-4-{[4-(dimethylamino)quinazolin-2- | 427 (M + H) | 3 |
| | yl]amino}cyclohexyl)methyl]thiourea | | |
| 587 | N-(3-chlorophenyl)-N′-[(cis-4-{[4-(dimethylamino)quinazolin-2- | 469 (M + H) | 3 |
| | yl]amino}cyclohexyl)methyl]thiourea | | |
| 588 | N-(4-chlorophenyl)-N′-[(cis-4-{[4-(dimethylamino)quinazolin-2- | 469 (M + H) | 3 |
| | yl]amino}cyclohexyl)methyl]thiourea | | |
| 589 | N-(2,4-dichlorophenyl)-N′-[(cis-4-{[4-(dimethylamino)- | 503 (M + H) | 3 |
| | quinazolin-2-yl]amino}cyclohexyl)methyl]thiourea | | |
| 590 | N-(2,4-dimethoxyphenyl)-N′-[(cis-4-{[4-(dimethylamino)- | 495 (M + H) | 3 |
| | quinazolin-2-yl]amino}cyclohexyl)methyl]thiourea | | |
| 591 | N-(2,5-difluorophenyl)-N′-[(cis-4-{[4-(dimethylamino)quinazolin- | 471 (M + H) | 3 |
| | 2-yl]amino}cyclohexyl)methyl]thiourea | | |
| 592 | N-(2,5-dichlorophenyl)-N′-[(cis-4-{[4-(dimethylamino)- | 503 (M + H) | 3 |
| | quinazolin-2-yl]amino}cyclohexyl)methyl]thiourea | | |
| 593 | N-[(cis-4-{[4-(dimethylamino)quinazolin-2- | 463 (M + H) | 2 |
| | yl]amino}cyclohexyl)methyl]-N′-(2,6-dimethylphenyl)thiourea | | |
| 594 | N-(3,4-dichlorophenyl)-N′-[(cis-4-{[4-(dimethylamino)- | 503 (M + H) | 3 |
| | quinazolin-2-yl]amino}cyclohexyl)methyl]thiourea | | |
| 595 | N-(2,6-dichlorophenyl)-N′-[(cis-4-{[4-(dimethylamino)- | 503 (M + H) | 2 |
| | quinazolin-2-yl]amino}cyclohexyl)methyl]thiourea | | |
| 596 | N-[(cis-4-{[4-(dimethylamino)quinazolin-2- | 479 (M + H) | 3 |
| | yl]amino}cyclohexyl)methyl]-N′-(4-ethoxyphenyl)thiourea | | |
| 597 | N-[(cis-4-{[4-(dimethylamino)quinazolin-2-yl]amino}- | 505 (M + H) | 2 |
| | cyclohexyl)methyl]-N′-(2-ethyl-6-isopropylphenyl)thiourea | | |
| 598 | N-[(cis-4-{[4-(dimethylamino)quinazolin-2- | 439 (M + H) | 3 |
| | yl]amino}cyclohexyl)methyl]-N′-(2-furylmethyl)thiourea | | |
| 599 | N-[(cis-4-{[4-(dimethylamino)quinazolin-2- | 453 (M + H) | 3 |
| | yl]amino}cyclohexyl)methyl]-N′-(4-fluorophenyl)thiourea | | |
| 600 | N-[(cis-4-{[4-(dimethylamino)quinazolin-2- | 443 (M + H) | 3 |
| | yl]amino}cyclohexyl)methyl]-N′-hexylthiourea | | |
| 601 | N-[(cis-4-{[4-(dimethylamino)quinazolin-2-yl]amino}- | 559 (M + H) | 3 |
| | cyclohexyl)methyl]-N′-[4-(trans-4-propylcyclohexyl)phenyl]- | | |
| | thiourea | | |
| 602 | N-[(cis-4-{[4-(dimethylamino)quinazolin-2- | 415 (M + H) | 2 |
| | yl]amino}cyclohexyl)methyl]-N′-isobutylthiourea | | |
| 603 | N-[(cis-4-{[4-(dimethylamino)quinazolin-2-yl]amino}- | 541 (M + H) | 3 |
| | cyclohexyl)methyl]-N′-(4-methoxybiphenyl-3-yl)thiourea | | |
| 604 | N-(1,3-benzodioxol-5-ylmethyl)-N′-[(cis-4-{[4-(dimethylamino)- | 493 (M + H) | 2 |
| | quinazolin-2-yl]amino}cyclohexyl)methyl]thiourea | | |
| 605 | N-[(cis-4-{[4-(dimethylamino)quinazolin-2- | 449 (M + H) | 3 |
| | yl]amino}cyclohexyl)methyl]-N′-(3-methylphenyl)thiourea | | |
| 606 | N-[(cis-4-{[4-(dimethylamino)quinazolin-2- | 481 (M + H) | 3 |
| | yl]amino}cyclohexyl)methyl]-N′-[4-(methylthio)phenyl]thiourea | | |
| 607 | N-[(cis-4-{[4-(dimethylamino)quinazolin-2- | 465 (M + H) | 3 |
| | yl]amino}cyclohexyl)methyl]-N′-(4-methoxyphenyl)thiourea | | |
| 608 | N-[(cis-4-{[4-(dimethylamino)quinazolin-2-yl]amino}- | 413 (M + H) | 3 |
| | cyclohexyl)methyl]-N′-(2-methylprop-2-en-1-yl)thiourea | | |
| 609 | N-[(cis-4-{[4-(dimethylamino)quinazolin-2- | 465 (M + H) | 3 |
| | yl]amino}cyclohexyl)methyl]-N′-(2-methoxyphenyl)thiourea | | |
| 610 | N-[(cis-4-{[4-(dimethylamino)quinazolin-2- | 373 (M + H) | 3 |
| | yl]amino}cyclohexyl)methyl]-N′-methylthiourea | | |
| 611 | N-[(cis-4-{[4-(dimethylamino)quinazolin-2- | 485 (M + H) | 3 |
| | yl]amino}cyclohexyl)methyl]-N′-1-naphthylthiourea | | |
| 612 | N-[(cis-4-{[4-(dimethylamino)quinazolin-2- | 480 (M + H) | 3 |
| | yl]amino}cyclohexyl)methyl]-N′-(3-nitrophenyl)thiourea | | |
| 613 | N-[(cis-4-{[4-(dimethylamino)quinazolin-2- | 480 (M + H) | 2 |
| | yl]amino}cyclohexyl)methyl]-N′-(4-nitrophenyl)thiourea | | |
| 614 | N-[(cis-4-{[4-(dimethylamino)quinazolin-2-yl]amino}- | 471 (M + H) | 3 |
| | cyclohexyl)methyl]-N′-(1,1,3,3-tetramethylbutyl)thiourea | | |
| 615 | N-[(cis-4-{[4-(dimethylamino)quinazolin-2- | 435 (M + H) | 3 |
| | yl]amino}cyclohexyl)methyl]-N′-phenylthiourea | | |
| 616 | N-[(cis-4-{[4-(dimethylamino)quinazolin-2- | 525 (M + H) | 2 |
| | yl]amino}cyclohexyl)methyl]-N′-(pentafluorophenyl)thiourea | | |
| 617 | N-[(cis-4-{[4-(dimethylamino)quinazolin-2- | 401 (M + H) | 3 |
| | yl]amino}cyclohexyl)methyl]-N′-propylthiourea | | |
| 618 | N-[(cis-4-{[4-(dimethylamino)quinazolin-2-yl]amino}- | 503 (M + H) | 3 |
| | cyclohexyl)methyl]-N′-[3-(trifluoromethyl)phenyl]thiourea | | |
| 619 | N-[(cis-4-{[4-(dimethylamino)quinazolin-2-yl]amino}- | 525 (M + H) | 3 |
| | cyclohexyl)methyl]-N′-(3,4,5-trimethoxyphenyl)thiourea | | |
| 620 | N-[(cis-4-{[4-(dimethylamino)quinazolin-2-yl]amino}- | 443 (M + H) | 2 |
| | cyclohexyl)methyl]-N′-(tetrahydrofuran-2-ylmethyl)thiourea | | |
| 621 | N-[(cis-4-{[4-(dimethylamino)quinazolin-2- | 449 (M + H) | 3 |
| | yl]amino}cyclohexyl)methyl]-N′-(4-methylphenyl)thiourea | | |
| 622 | N-[(cis-4-{[4-(dimethylamino)quinazolin-2- | 449 (M + H) | 3 |
| | yl]amino}cyclohexyl)methyl]-N′-(2-methylphenyl)thiourea | | |
| 623 | N-(tert-butyl)-N′-[(cis-4-{[4-(dimethylamino)quinazolin-2- | 415 (M + H) | 3 |
| | yl]amino}cyclohexyl)methyl]thiourea | | |
| 624 | N-1-adamantyl-N′-[(cis-4-{[4-(dimethylamino)quinazolin-2- | 493 (M + H) | 3 |
| | yl]amino}cyclohexyl)methyl]thiourea | | |
| 625 | N-(2-bromophenyl)-N′-[(cis-4-{[4-(dimethylamino)quinazolin-2- | 513 (M + H) | 3 |
| | yl]amino}cyclohexyl)methyl]thiourea | | |
| 626 | N-(2-chlorophenyl)-N′-[(cis-4-{[4-(dimethylamino)quinazolin-2- | 469 (M + H) | 3 |
| | yl]amino}cyclohexyl)methyl]thiourea | | |
| 627 | N-[(cis-4-{[4-(dimethylamino)quinazolin-2- | 463 (M + H) | 3 |
| | yl]amino}cyclohexyl)methyl]-N′-(2-phenylethyl)thiourea | | |
| 628 | N-(3,4-dimethoxyphenyl)-N′-[(cis-4-{[4-(dimethylamino)- | 495 (M + H) | 3 |
| | quinazolin-2-yl]amino}cyclohexyl)methyl]thiourea | | |
| 629 | N-[(cis-4-{[4-(dimethylamino)quinazolin-2- | 463 (M + H) | 3 |
| | yl]amino}cyclohexyl)methyl]-N′-(4-ethylphenyl)thiourea | | |
| 630 | N-[(cis-4-{[4-(dimethylamino)quinazolin-2- | 481 (M + H) | 3 |
| | yl]amino}cyclohexyl)methyl]-N′-[2-(methylthio)phenyl]thiourea | | |
| 631 | N-[(cis-4-{[4-(dimethylamino)quinazolin-2-yl]amino}- | 519 (M + H) | 2 |
| | cyclohexyl)methyl]-N′-[2-(trifluoromethoxy)phenyl]thiourea | | |
| 632 | N-[(cis-4-{[4-(dimethylamino)quinazolin-2-yl]amino}- | 503 (M + H) | 3 |
| | cyclohexyl)methyl]-N′-[2-(trifluoromethyl)phenyl]thiourea | | |
| 633 | N-[(cis-4-{[4-(dimethylamino)quinazolin-2- | 489 (M + H) | 2 |
| | yl]amino}cyclohexyl)methyl]-N′-(2,3,4-trifluorophenyl)thiourea | | |
| 634 | N-(2,3-dichlorophenyl)-N′-[(cis-4-{[4-(dimethylamino)- | 503 (M + H) | 3 |
| | quinazolin-2-yl]amino}cyclohexyl)methyl]thiourea | | |
| 635 | N-(2,4-difluorophenyl)-N′-[(cis-4-{[4-(dimethylamino)quinazolin- | 471 (M + H) | 3 |
| | 2-yl]amino}cyclohexyl)methyl]thiourea | | |
| 636 | N-(2,5-dimethoxyphenyl)-N′-[(cis-4-{[4-(dimethylamino)- | 495 (M + H) | 3 |
| | quinazolin-2-yl]amino}cyclohexyl)methyl]thiourea | | |
| 637 | N-(2,6-difluorophenyl)-N′-[(cis-4-{[4-(dimethylamino)quinazolin- | 471 (M + H) | 3 |
| | 2-yl]amino}cyclohexyl)methyl]thiourea | | |
| 638 | N-(2-chloro-4-nitrophenyl)-N′-[(cis-4-{[4-(dimethylamino)- | 514 (M + H) | 3 |
| | quinazolin-2-yl]amino}cyclohexyl)methyl]thiourea | | |
| 639 | N-[2-(difluoromethoxy)phenyl]-N′-[(cis-4-{[4-(dimethylamino)- | 501 (M + H) | 3 |
| | quinazolin-2-yl]amino}cyclohexyl)methyl]thiourea | | |
| 640 | N-[(cis-4-{[4-(dimethylamino)quinazolin-2- | 463 (M + H) | 2 |
| | yl]amino}cyclohexyl)methyl]-N′-(2-ethylphenyl)thiourea | | |
| 641 | N-[(cis-4-{[4-(dimethylamino)quinazolin-2-yl]amino}- | 521 (M + H) | 3 |
| | cyclohexyl)methyl]-N′-[2-fluoro-5-(trifluoromethyl)phenyl]- | | |
| | thiourea | | |
| 642 | N-[(cis-4-{[4-(dimethylamino)quinazolin-2- | 453 (M + H) | 3 |
| | yl]amino}cyclohexyl)methyl]-N′-(2-fluorophenyl)thiourea | | |
| 643 | N-[(cis-4-{[4-(dimethylamino)quinazolin-2- | 561 (M + H) | 3 |
| | yl]amino}cyclohexyl)methyl]-N′-(2-iodophenyl)thiourea | | |
| 644 | N-[(cis-4-{[4-(dimethylamino)quinazolin-2-yl]amino}- | 510 (M + H) | 3 |
| | cyclohexyl)methyl]-N′-(2-methoxy-4-nitrophenyl)thiourea | | |
| 645 | N-[(cis-4-{[4-(dimethylamino)quinazolin-2-yl]amino}- | 479 (M + H) | 3 |
| | cyclohexyl)methyl]-N′-(2-methoxy-5-methylphenyl)thiourea | | |
| 646 | N-[(cis-4-{[4-(dimethylamino)quinazolin-2-yl]amino}- | 535 (M + H) | 3 |
| | cyclohexyl)methyl]-N′-{3-[(trifluoromethyl)thio]phenyl}thiourea | | |
| 647 | N-(3,5-dichlorophenyl)-N′-[(cis-4-{[4-(dimethylamino)- | 503 (M + H) | 3 |
| | quinazolin-2-yl]amino}cyclohexyl)methyl]thiourea | | |
| 648 | N-(3,5-difluorophenyl)-N′-[(cis-4-{[4-(dimethylamino)quinazolin- | 471 (M + H) | 3 |
| | 2-yl]amino}cyclohexyl)methyl]thiourea | | |
| 649 | N-(3-ciyanophenyl)-N′-[(cis-4-{[4-(dimethylamino)quinazolin-2- | 460 (M + H) | 3 |
| | yl]amino}cyclohexyl)methyl]thiourea | | |
| 650 | N-[(cis-4-{[4-(dimethylamino)quinazolin-2- | 453 (M + H) | 3 |
| | yl]amino}cyclohexyl)methyl]-N′-(3-fluorophenyl)thiourea | | |
| 651 | N-[(cis-4-{[4-(dimethylamino)quinazolin-2- | 561 (M + H) | 3 |
| | yl]amino}cyclohexyl)methyl]-N′-(3-iodophenyl)thiourea | | |
| 652 | N-[(cis-4-{[4-(dimethylamino)quinazolin-2- | 465 (M + H) | 3 |
| | yl]amino}cyclohexyl)methyl]-N′-(3-methoxyphenyl)thiourea | | |
| 653 | N-[4-(difluoromethoxy)phenyl]-N′-[(cis-4-{[4-(dimethylamino)- | 501 (M + H) | 3 |
| | quinazolin-2-yl]amino}cyclohexyl)methyl]thiourea | | |
| 654 | N-[(cis-4-{[4-(dimethylamino)quinazolin-2-yl]amino}- | 519 (M + H) | 3 |
| | cyclohexyl)methyl]-N′-[4-(trifluoromethoxy)phenyl]thiourea | | |
| 655 | N-[(cis-4-{[4-(dimethylamino)quinazolin-2-yl]amino}- | 503 (M + H) | 3 |
| | cyclohexyl)methyl]-N′-[4-(trifluoromethyl)phenyl]thiourea | | |
| 656 | N-[(cis-4-{[4-(dimethylamino)quinazolin-2-yl]amino}- | 535 (M + H) | 3 |
| | cyclohexyl)methyl]-N′-{4-[(trifluoromethyl)thio]phenyl}thiourea | | |
| 657 | N-(4-bromo-2-chlorophenyl)-N′-[(cis-4-{[4-(dimethylamino)- | 547 (M + H) | 3 |
| | quinazolin-2-yl]amino}cyclohexyl)methyl]thiourea | | |
| 658 | N-(4-bromo-2-fluorophenyl)-N′-[(cis-4-{[4-(dimethylamino)- | 531 (M + H) | 3 |
| | quinazolin-2-yl]amino}cyclohexyl)methyl]thiourea | | |
| 659 | N-[4-chloro-3-(trifluoromethyl)phenyl]-N′-[(cis-4-{[4-(dimethylamino) | 537 (M + H) | 3 |
| | quinazolin-2-yl]amino}cyclohexyl)methyl]thiourea | | |
| 660 | N-[(cis-4-{[4-(dimethylamino)quinazolin-2-yl]amino}cyclohexyl) | 521 (M + H) | 3 |
| | methyl]-N′-[4-fluoro-3-(trifluoromethyl)phenyl]thiourea | | |
| 661 | N-[(cis-4-{[4-(dimethylamino)quinazolin-2- | 561 (M + H) | 3 |
| | yl]amino}cyclohexyl)methyl]-N′-(4-iodophenyl)thiourea | | |
| 662 | N-(5-chloro-2-methylphenyl)-N′-[(cis-4-{[4-(dimethylamino)- | 483 (M + H) | 2 |
| | quinazolin-2-yl]amino}cyclohexyl)methyl]thiourea | | |
| 663 | N-[(1S,4R)-bicyclo[2.2.1]hept-2-yl]-N′-[(cis-4-{[4-(dimethylamino) | 453 (M + H) | 2 |
| | quinazolin-2-yl]amino}cyclohexyl)methyl]thiourea | | |
| 664 | tert-butyl {4-[({[(cis-4-{[4-(dimethylamino)quinazolin-2- | 550 (M + H) | 3 |
| | yl]amino}cyclohexyl)methyl]amino}carbonothioyl)amino]phenyl}- | | |
| | carbamate | | |
| 665 | N-[2-(3,4-dimethoxyphenyl)ethyl]-N′-[(cis-4-{[4-(dimethylamino) | 523 (M + H) | 2 |
| | quinazolin-2-yl]amino}cyclohexyl)methyl]thiourea | | |
| 666 | N-[2-(4-chlorophenyl)ethyl]-N′-[(cis-4-{[4-(dimethylamino)- | 497 (M + H) | 3 |
| | quinazolin-2-yl]amino}cyclohexyl)methyl]thiourea | | |
| 667 | N-[(cis-4-{[4-(dimethylamino)quinazolin-2-yl]amino}- | 571 (M + H) | 3 |
| | cyclohexyl)methyl]-N′-(2,3,4,5-tetrachlorophenyl)thiourea | | |
| 668 | N-[(cis-4-{[4-(dimethylamino)quinazolin-2- | 537 (M + H) | 3 |
| | yl]amino}cyclohexyl)methyl]-N′-(2,4,5-trichlorophenyl)thiourea | | |
| 669 | N-[(cis-4-{[4-(dimethylamino)quinazolin-2- | 668 (M + H) | 2 |
| | yl]amino}cyclohexyl)methyl]-N′-(2,4,6-tribromophenyl)thiourea | | |
| 670 | N-[(cis-4-{[4-(dimethylamino)quinazolin-2- | 537 (M + H) | 2 |
| | yl]amino}cyclohexyl)methyl]-N′-(2,4,6-trichlorophenyl)thiourea | | |
| 671 | N-[(cis-4-{[4-(dimethylamino)quinazolin-2- | 489 (M + H) | 3 |
| | yl]amino}cyclohexyl)methyl]-N′-(2,4,6-trifluorophenyl)thiourea | | |
| 672 | N-[(cis-4-{[4-(dimethylamino)quinazolin-2- | 477 (M + H) | 2 |
| | yl]amino}cyclohexyl)methyl]-N′-mesitylthiourea | | |
| 673 | N-[(cis-4-{[4-(dimethylamino)quinazolin-2- | 463 (M + H) | 3 |
| | yl]amino}cyclohexyl)methyl]-N′-(2,4-dimethylphenyl)thiourea | | |
| 674 | N-(2,6-diethylphenyl)-N′-[(cis-4-{[4-(dimethylamino)quinazolin- | 491 (M + H) | 1 |
| | 2-yl]amino}cyclohexyl)methyl]thiourea | | |
| 675 | N-(2,6-diisopropylphenyl)-N′-[(cis-4-{[4-(dimethylamino)- | 519 (M + H) | 2 |
| | quinazolin-2-yl]amino}cyclohexyl)methyl]thiourea | | |
| 676 | N-(2-bromo-4-methylphenyl)-N′-[(cis-4-{[4-(dimethylamino)- | 527 (M + H) | 3 |
| | quinazolin-2-yl]amino}cyclohexyl)methyl]thiourea | | |
| 677 | N-[2-chloro-5-(trifluoromethyl)phenyl]-N′-[(cis-4-{[4-(dimethylamino) | 537 (M + H) | 3 |
| | quinazolin-2-yl]amino}cyclohexyl)methyl]thiourea | | |
| 678 | N-(2-chlorobenzyl)-N′-[(cis-4-{[4-(dimethylamino)quinazolin-2- | 483 (M + H) | 3 |
| | yl]amino}cyclohexyl)methyl]thiourea | | |
| 679 | N-[(cis-4-{[4-(dimethylamino)quinazolin-2-yl]amino}- | 477 (M + H) | 2 |
| | cyclohexyl)methyl]-N′-(2-ethyl-6-methylphenyl)thiourea | | |
| 680 | N-[(cis-4-{[4-(dimethylamino)quinazolin-2- | 477 (M + H) | 2 |
| | yl]amino}cyclohexyl)methyl]-N′-(2-isopropylphenyl)thiourea | | |
| 681 | N-[(cis-4-{[4-(dimethylamino)quinazolin-2- | 481 (M + H) | 3 |
| | yl]amino}cyclohexyl)methyl]-N′-[3-(methylthio)phenyl]thiourea | | |
| 682 | N-(3,4-dichlorobenzyl)-N′-[(cis-4-{[4-(dimethylamino)- | 517 (M + H) | 3 |
| | quinazolin-2-yl]amino}cyclohexyl)methyl]thiourea | | |
| 683 | N-(3,5-dimethoxyphenyl)-N′-[(cis-4-{[4-(dimethylamino)- | 495 (M + H) | 3 |
| | quinazolin-2-yl]amino}cyclohexyl)methyl]thiourea | | |
| 684 | N-[(cis-4-{[4-(dimethylamino)quinazolin-2- | 463 (M + H) | 3 |
| | yl]amino}cyclohexyl)methyl]-N′-(3,5-dimethylphenyl)thiourea | | |
| 685 | N-[3-(benzyloxy)phenyl]-N′-[(cis-4-{[4-(dimethylamino)- | 541 (M + H) | 3 |
| | quinazolin-2-yl]amino}cyclohexyl)methyl]thiourea | | |
| 686 | 3-[({[(cis-4-{[4-(dimethylamino)quinazolin-2-yl]amino}- | 479 (M + H) | 3 |
| | cyclohexyl)methyl]amino}carbonothioyl)amino]benzoic acid | | |
| 687 | N-(3-chloro-4-methylphenyl)-N′-[(cis-4-{[4-(dimethylamino)- | 483 (M + H) | 3 |
| | quinazolin-2-yl]amino}cyclohexyl)methyl]thiourea | | |
| 688 | methyl 3-[({[(cis-4-{[4-(dimethylamino)quinazolin-2-yl]amino}- | 493 (M + H) | 3 |
| | cyclohexyl)methyl]amino}carbonothioyl)amino]benzoate | | |
| 689 | N-[(cis-4-{[4-(dimethylamino)quinazolin-2- | 477 (M + H) | 3 |
| | yl]amino}cyclohexyl)methyl]-N′-(3-phenylpropyl)thiourea | | |
| 690 | N-[4-(benzyloxy)phenyl]-N′-[(cis-4-{[4-(dimethylamino)- | 541 (M + H) | 3 |
| | quinazolin-2-yl]amino}cyclohexyl)methyl]thiourea | | |
| 691 | N-(4-bromo-2,6-dimethylphenyl)-N′-[(cis-4-{[4-(dimethylamino) | 541 (M + H) | 1 |
| | quinazolin-2-yl]amino}cyclohexyl)methyl]thiourea | | |
| 692 | N-(4-bromo-2-methylphenyl)-N′-[(cis-4-{[4-(dimethylamino)- | 527 (M + H) | 3 |
| | quinazolin-2-yl]amino}cyclohexyl)methyl]thiourea | | |
| 693 | N-[4-bromo-2-(trifluoromethyl)phenyl]-N′-[(cis-4-{[4-(dimethylamino) | 581 (M + H) | 2 |
| | quinazolin-2-yl]amino}cyclohexyl)methyl]thiourea | | |
| 694 | N-(4-chloro-2-methylphenyl)-N′-[(cis-4-{[4-(dimethylamino)- | 483 (M + H) | 3 |
| | quinazolin-2-yl]amino}cyclohexyl)methyl]thiourea | | |
| 695 | N-(4-chlorobenzyl)-N′-[(cis-4-([4-(dimethylamino)quinazolin-2- | 483 (M + H) | 3 |
| | yl]amino)cyclohexyl)methyl]thiourea | | |
| 696 | ethyl 4-[({[(cis-4-{[4-(dimethylamino)quinazolin-2-yl]amino}- | 507 (M + H) | 3 |
| | cyclohexyl)methyl]amino}carbonothioyl)amino]benzoate | | |
| 697 | N-[(cis-4-{[4-(dimethylamino)quinazolin-2-yl]amino}- | 481 (M + H) | 2 |
| | cyclohexyl)methyl]-N′-[1-(4-fluorophenyl)ethyl]thiourea | | |
| 698 | N-[(cis-4-{[4-(dimethylamino)quinazolin-2- | 467 (M + H) | 3 |
| | yl]amino}cyclohexyl)methyl]-N′-(4-fluorobenzyl)thiourea | | |
| 699 | N-[(cis-4-{[4-(dimethylamino)quinazolin-2- | 477 (M + H) | 3 |
| | yl]amino}cyclohexyl)methyl]-N′-(4-isopropylphenyl)thiourea | | |
| 700 | N-[(cis-4-{[4-(dimethylamino)quinazolin-2-yl]amino}- | 510 (M + H) | 3 |
| | cyclohexyl)methyl]-N′-(4-methoxy-2-nitrophenyl)thiourea | | |
| 701 | N-[(cis-4-{[4-(dimethylamino)quinazolin-2- | 479 (M + H) | 3 |
| | yl]amino}cyclohexyl)methyl]-N′-(4-methoxybenzyl)thiourea | | |
| 702 | methyl 4-[({[(cis-4-{[4-(dimethylamino)quinazolin-2-yl]amino}- | 493 (M + H) | 3 |
| | cyclohexyl)methyl]amino}carbonothioyl)amino]benzoate | | |
| 703 | N-[(cis-4-{[4-(dimethylamino)quinazolin-2-yl]amino}- | 494 (M + H) | 3 |
| | cyclohexyl)methyl]-N′-(4-methyl-2-nitrophenyl)thiourea | | |
| 704 | N-[(cis-4-{[4-(dimethylamino)quinazolin-2- | 463 (M + H) | 3 |
| | yl]amino}cyclohexyl)methyl]-N′-(4-methylbenzyl)thiourea | | |
| 705 | N-(4-butylphenyl)-N′-[(cis-4-{[4-(dimethylamino)quinazolin-2- | 491 (M + H) | 3 |
| | yl]amino}cyclohexyl)methyl]thiourea | | |
| 706 | N-(5-chloro-2-methoxyphenyl)-N′-[(cis-4-{[4-(dimethylamino)- | 499 (M + H) | 2 |
| | quinazolin-2-yl]amino}cyclohexyl)methyl]thiourea | | |
| 707 | N-[(cis-4-{[4-(dimethylamino)quinazolin-2- | 463 (M + H) | 3 |
| | yl]amino}cyclohexyl)methyl]-N′-(1-phenylethyl)thiourea | | |
| 708 | N-[(cis-4-{[4-(dimethylamino)quinazolin-2- | 525 (M + H) | 2 |
| | yl]amino}cyclohexyl)methyl]-N′-(diphenylmethyl)thiourea | | |
| 709 | N-cyclododecyl-N′-[(cis-4-{[4-(dimethylamino)quinazolin-2- | 525 (M + H) | 2 |
| | yl]amino}cyclohexyl)methyl]thiourea | | |
| 710 | N-(cyclohexylmethyl)-N′-[(cis-4-{[4-(dimethylamino)quinazolin- | 455 (M + H) | 2 |
| | 2-yl]amino}cyclohexyl)methyl]thiourea | | |
| 711 | N-cyclooctyl-N′-[(cis-4-{[4-(dimethylamino)quinazolin-2- | 469 (M + H) | 3 |
| | yl]amino}cyclohexyl)methyl]thiourea | | |
| 712 | N-cyclopropyl-N′-[(cis-4-{[4-(dimethylamino)quinazolin-2- | 399 (M + H) | 3 |
| | yl]amino}cyclohexyl)methyl]thiourea | | |
| 713 | N-[(cis-4-{[4-(dimethylamino)quinazolin-2- | 499 (M + H) | 3 |
| | yl]amino}cyclohexyl)methyl]-N′-(1-naphthylmethyl)thiourea | | |
| 714 | N-[(cis-4-{[4-(dimethylamino)quinazolin-2- | 539 (M + H) | 3 |
| | yl]amino}cyclohexyl)methyl]-N′-(2,2-diphenylethyl)thiourea | | |
| 715 | N-[(cis-4-{[4-(dimethylamino)quinazolin-2-yl]amino}- | 571 (M + H) | 1 |
| | cyclohexyl)methyl]-N′-(2,3,5,6-tetrachlorophenyl)thiourea | | |
| 716 | N-(2,3-dimethoxybenzyl)-N′-[(cis-4-{[4-(dimethylamino)- | 509 (M + H) | 2 |
| | quinazolin-2-yl]amino}cyclohexyl)methyl]thiourea | | |
| 717 | N-[(cis-4-{[4-(dimethylamino)quinazolin-2- | 477 (M + H) | 3 |
| | yl]amino}cyclohexyl)methyl]-N′-(2,4,5-trimethylphenyl)thiourea | | |
| 718 | N-(2,4-dichlorobenzyl)-N′-[(cis-4-{[4-(dimethylamino)- | 517 (M + H) | 2 |
| | quinazolin-2-yl]amino}cyclohexyl)methyl]thiourea | | |
| 719 | N-(2,5-dibromophenyl)-N′-[(cis-4-{[4-(dimethylamino)- | 591 (M + H) | 3 |
| | quinazolin-2-yl]amino}cyclohexyl)methyl]thiourea | | |
| 720 | N-[2-(2,5-dimethoxyphenyl)ethyl]-N′-[(cis-4-{[4-(dimethylamino) | 523 (M + H) | 3 |
| | quinazolin-2-yl]amino}cyclohexyl)methyl]thiourea | | |
| 721 | N-biphenyl-2-yl-N′-[(cis-4-{[4-(dimethylamino)quinazolin-2- | 511 (M + H) | 3 |
| | yl]amino}cyclohexyl)methyl]thiourea | | |
| 722 | N-(2-chloro-5-nitrophenyl)-N′-[(cis-4-{[4-(dimethylamino)- | 514 (M + H) | 3 |
| | quinazolin-2-yl]amino}cyclohexyl)methyl]thiourea | | |
| 723 | N-(2-cyanophenyl)-N′-[(cis-4-{[4-(dimethylamino)quinazolin-2- | 460 (M + H) | 3 |
| | yl]amino}cyclohexyl)methyl]thiourea | | |
| 724 | N-[(cis-4-{[4-(dimethylamino)quinazolin-2- | 467 (M + H) | 3 |
| | yl]amino}cyclohexyl)methyl]-N′-(2-fluorobenzyl)thiourea | | |
| 725 | N-[(cis-4-{[4-(dimethylamino)quinazolin-2-yl]amino}- | 510 (M + H) | 2 |
| | cyclohexyl)methyl]-N′-(2-methoxy-5-nitrophenyl)thiourea | | |
| 726 | N-[(cis-4-{[4-(dimethylamino)quinazolin-2-yl]amino}- | 494 (M + H) | 3 |
| | cyclohexyl)methyl]-N′-(2-methyl-4-nitrophenyl)thiourea | | |
| 727 | N-[(cis-4-{[4-(dimethylamino)quinazolin-2- | 463 (M + H) | 3 |
| | yl]amino}cyclohexyl)methyl]-N′-(2-methylbenzyl)thiourea | | |
| 728 | N-(3,4-dimethoxybenzyl)-N′-[(cis-4-{[4-(dimethylamino)- | 509 (M + H) | 3 |
| | quinazolin-2-yl]amino}cyclohexyl)methyl]thiourea | | |
| 729 | N-(3-chlorobenzyl)-N′-[(cis-4-{[4-(dimethylamino)quinazolin-2- | 483 (M + H) | 3 |
| | yl]amino}cyclohexyl)methyl]thiourea | | |
| 730 | ethyl 3-[({[(cis-4-{[4-(dimethylamino)quinazolin-2-yl]amino}- | 507 (M + H) | 3 |
| | cyclohexyl)methyl]amino}carbonothioyl)amino]benzoate | | |
| 731 | N-[(cis-4-{[4-(dimethylamino)quinazolin-2- | 463 (M + H) | 3 |
| | yl]amino}cyclohexyl)methyl]-N′-(3-ethylphenyl)thiourea | | |
| 732 | N-[(cis-4-{[4-(dimethylamino)quinazolin-2- | 467 (M + H) | 3 |
| | yl]amino}cyclohexyl)methyl]-N′-(3-fluorobenzyl)thiourea | | |
| 733 | N-[(cis-4-{[4-(dimethylamino)quinazolin-2- | 479 (M + H) | 3 |
| | yl]amino}cyclohexyl)methyl]-N′-(3-methoxybenzyl)thiourea | | |
| 734 | N-[(cis-4-{[4-(dimethylamino)quinazolin-2- | 463 (M + H) | 3 |
| | yl]amino}cyclohexyl)methyl]-N′-(3-methylbenzyl)thiourea | | |
| 735 | N-(4-bromo-3-chlorophenyl)-N′-[(cis-4-{[4-(dimethylamino)- | 547 (M + H) | 3 |
| | quinazolin-2-yl]amino}cyclohexyl)methyl]thiourea | | |
| 736 | N-(4-bromo-3-methylphenyl)-N′-[(cis-4-{[4-(dimethylamino)- | 527 (M + H) | 3 |
| | quinazolin-2-yl]amino}cyclohexyl)methyl]thiourea | | |
| 737 | N-[4-chloro-2-(trifluoromethyl)phenyl]-N′-[(cis-4-{[4-(dimethylamino) | 537 (M + H) | 3 |
| | quinazolin-2-yl]amino}cyclohexyl)methyl]thiourea | | |
| 738 | N-(4-decylphenyl)-N′-[(cis-4-{[4-(dimethylamino)quinazolin-2- | 575 (M + H) | 3 |
| | yl]amino}cyclohexyl)methyl]thiourea | | |
| 739 | N-[(cis-4-{[4-(dimethylamino)quinazolin-2-yl]amino}- | 467 (M + H) | 3 |
| | cyclohexyl)methyl]-N′-(4-fluoro-2-methylphenyl)thiourea | | |
| 740 | N-[(cis-4-{[4-(dimethylamino)quinazolin-2-yl]amino}- | 572 (M + H) | 3 |
| | cyclohexyl)methyl]-N′-[4-(4-nitrophenoxy)phenyl]thiourea | | |
| 741 | N-[(cis-4-{[4-(dimethylamino)quinazolin-2-yl]amino}- | 588 (M + H) | 3 |
| | cyclohexyl)methyl]-N′-{4-[(4-nitrophenyl)thio]phenyl}thiourea | | |
| 742 | 4-[({[(cis-4-{[4-(dimethylamino)quinazolin-2-yl]amino}- | 514 (M + H) | 3 |
| | cyclohexyl)methyl]amino}carbonothioyl)amino]benzenesulfonamide | | |
| 743 | N-[(cis-4-{[4-(dimethylamino)quinazolin-2-yl]amino}- | 479 (M + H) | 2 |
| | cyclohexyl)methyl]-N′-(4-methoxy-2-methylphenyl)thiourea | | |
| 744 | N-[(cis-4-{[4-(dimethylamino)quinazolin-2-yl]amino}- | 477 (M + H) | 3 |
| | cyclohexyl)methyl]-N′-[2-(4-methylphenyl)ethyl]thiourea | | |
| 745 | N-[(cis-4-{[4-(dimethylamino)quinazolin-2- | 527 (M + H) | 3 |
| | yl]amino}cyclohexyl)methyl]-N′-(4-phenoxyphenyl)thiourea | | |
| 746 | N-(5-chloro-2,4-dimethoxyphenyl)-N′-[(cis-4-{[4-(dimethylamino) | 529 (M + H) | 3 |
| | quinazolin-2-yl]amino}cyclohexyl)methyl]thiourea | | |
| 747 | N-(2,3-dihydro-1H-inden-5-yl)-N′-[(cis-4-{[4-(dimethylamino) | 475 (M + H) | 3 |
| | quinazolin-2-yl]amino}cyclohexyl)methyl]thiourea | | |
| 748 | N-cycloheptyl-N′-[(cis-4-{[4-(dimethylamino)quinazolin-2- | 455 (M + H) | 3 |
| | yl]amino}cyclohexyl)methyl]thiourea | | |
| 749 | N-[(cis-4-{[4-(dimethylamino)quinazolin-2- | 463 (M + H) | 3 |
| | yl]amino}cyclohexyl)methyl]-N′-[(1R)-1-phenylethyl]thiourea | | |
| 750 | butyl 2-[({[(cis-4-{[4-(dimethylamino)quinazolin-2- | 519 (M + H) | 3 |
| | yl]amino}cyclohexyl)methyl]amino}carbonyl)amino]benzoate | | |
| 751 | dimethyl 5-[({[(cis-4-{[4-(dimethylamino)quinazolin-2- | 535 (M + H) | 3 |
| | yl]amino}cyclohexyl)methyl]amino}carbonyl)amino]isophthalate | | |
| 752 | N-[(cis-4-{[4-(dimethylamino)quinazolin-2-yl]amino}- | 503 (M + H) | 3 |
| | cyclohexyl)methyl]-N′-[4-(trifluoromethoxy)phenyl]urea | | |
| 753 | N-(4-bromo-2,6-dimethylphenyl)-N′-[(cis-4-{[4- | 525 (M + H) | 1 |
| | (dimethylamino)quinazolin-2-yl]amino}cyclohexyl)methyl]urea | | |
| 754 | N-(4-bromo-2-methylphenyl)-N′-[(cis-4-{[4- | 511 (M + H) | 2 |
| | (dimethylamino)quinazolin-2-yl]amino}cyclohexyl)methyl]urea | | |
| 755 | N-[(cis-4-{[4-(dimethylamino)quinazolin-2-yl]amino}- | 549 (M + H) | 3 |
| | cyclohexyl)methyl]-N′-(2,2,4,4,-tetrafluoro-4H-1,3-benzodioxin- | | |
| | 6-yl)urea | | |
| 756 | ethyl N-({[(cis-4-{[4-(dimethylamino)quinazolin-2- | 519 (M + H) | 3 |
| | yl]amino}cyclohexyl)methyl]amino}carbonyl)phenylalaninate | | |
| 757 | N-[(cis-4-{[4-(dimethylamino)quinazolin-2- | 453 (M + H) | 3 |
| | yl]amino}cyclohexyl)methyl]-N′-[2-(2-thienyl)ethyl]urea | | |
| 758 | N-(2,3-dihydro-1,4-benzodioxin-6-yl)-N′-[(cis-4-{[4- | 477 (M + H) | 3 |
| | (dimethylamino)quinazolin-2-yl]amino}cyclohexyl)methyl]urea | | |
| 759 | N-(2,6-dibromo-4-isopropylphenyl)-N′-[(cis-4-{[4- | 617 (M + H) | 2 |
| | (dimethylamino)quinazolin-2-yl]amino}cyclohexyl)methyl]urea | | |
| 760 | N-(2-cyanophenyl)-N′-[(cis-4-{[4-(dimethylamino)quinazolin-2- | 444 (M + H) | 3 |
| | yl]amino}cyclohexyl)methyl]urea | | |
| 761 | N-[(cis-4-{[4-(dimethylamino)quinazolin-2- | 425 (M + H) | 3 |
| | yl]amino}cyclohexyl)methyl]-N′-2-thienylurea | | |
| 762 | N-[3-(cyclopentyloxy)-4-methoxyphenyl]-N′-[(cis-4-{[4- | 533 (M + H) | 3 |
| | (dimethylamino)quinazolin-2-yl]amino}cyclohexyl)methyl]urea | | |
| 763 | N-(3,4-dihydro-2H-1,5-benzodioxepin-7-yl)-N′-[(cis-4-{[4- | 491 (M + H) | 3 |
| | (dimethylamino)quinazolin-2-yl]amino}cyclohexyl)methyl]urea | | |
| 764 | N-[(cis-4-{[4-(dimethylamino)quinazolin-2- | 425 (M + H) | 2 |
| | yl]amino}cyclohexyl)methyl]-N′-3-thienylurea | | |
| 765 | N-(4-tert-butylphenyl)-N′-[(cis-4-{[4-(dimethylamino)quinazolin- | 475 (M + H) | 3 |
| | 2-yl]amino}cyclohexyl)methyl]urea | | |
| 766 | N-(4-butyl-2-methylphenyl)-N′-[(cis-4-{[4- | 489 (M + H) | 3 |
| | (dimethylamino)quinazolin-2-yl]amino}cyclohexyl)methyl]urea | | |
| 767 | N-[(cis-4-{[4-(dimethylamino)quinazolin-2-yl]amino}cyclohexyl) | 491 (M + H) | 1 |
| | methyl]-N′-[5-methyl-2-(trifluoromethyl)-3-furyl]urea | | |
| 768 | N-[(cis-4-{[4-(dimethylamino)quinazolin-2- | 501 (M + H) | 3 |
| | yl]amino}cyclohexyl)methyl]-N′-(5-phenyl-2-thienyl)urea | | |
| 769 | N-[(cis-4-{[4-(dimethylamino)quinazolin-2-yl]amino}cyclohexyl) | 495 (M + H) | 2 |
| | methyl]-N′-(6-fluoro-4H-1,3-benzodioxin-8-yl)urea | | |
| 770 | benzyl 4-[({[(cis-4-{[4-(dimethylamino)quinazolin-2- | 560 (M + H) | 3 |
| | yl]amino}cyclohexyl)methyl]amino}carbonyl)amino]piperidine- | | |
| | 1-carboxylate | | |
| 771 | N-[(cis-4-{[4-(dimethylamino)quinazolin-2-yl]amino}- | 566 (M + H) | 3 |
| | cyclohexyl)methyl]-N′-[4-(6-methyl-1,3-benzothiazol-2-yl)- | | |
| | phenyl]urea | | |
| 772 | N-[4-(dimethylamino)phenyl]-N′-[(cis-4-{[4- | 462 (M + H) | 3 |
| | (dimethylamino)quinazolin-2-yl]amino}cyclohexyl)methyl]urea | | |
| 773 | N-(2,6-dichloropyridin-4-yl)-N′-[(cis-4-{[4- | 488 (M + H) | 3 |
| | (dimethylamino)quinazolin-2-yl]amino}cyclohexyl)methyl]urea | | |
| 774 | N-[(cis-4-{[4-(dimethylamino)quinazolin-2- | 438 (M + H) | 2 |
| | yl]amino}cyclohexyl)methyl]-N′-(3,5-dimethylisoxazol-4-yl)urea | | |
| 775 | N-[(cis-4-{[4-(dimethylamino)quinazolin-2-yl]amino}- | 500 (M + H) | 1 |
| | cyclohexyl)methyl]-N′-(3-methyl-5-phenylisoxazol-4-yl)urea | | |
| 776 | N-[(cis-4-{[4-(dimethylamino)quinazolin-2-yl]amino}- | 500 (M + H) | 2 |
| | cyclohexyl)methyl]-N′-(5-methyl-3-phenylisoxazol-4-yl)urea | | |
| 777 | N-[(cis-4-{[4-(dimethylamino)quinazolin-2- | 397 (M + H) | 3 |
| | yl]amino}cyclohexyl)methyl]-N′-prop-2-yn-1-ylthiourea | | |
| 778 | N-[(cis-4-{[4-(dimethylamino)quinazolin-2-yl]amino}- | 582 (M + H) | 3 |
| | cyclohexyl)methyl]-N′-[4-(piperidin-1-ylsulfonyl)phenyl]thiourea | | |
| 779 | N-(2-cyclohex-1-en-1-ylethyl)-N′-[(cis-4-{[4-(dimethylamino)- | 467 (M + H) | 3 |
| | quinazolin-2-yl]amino}cyclohexyl)methyl]thiourea | | |
| 780 | N-[(cis-4-{[4-(dimethylamino)quinazolin-2- | 463 (M + H) | 3 |
| | yl]amino}cyclohexyl)methyl]-N′-(2,3-dimethylphenyl)thiourea | | |
| 781 | N-(2,4-dibromo-6-fluorophenyl)-N′-[(cis-4-{[4-(dimethylamino) | 609 (M + H) | 2 |
| | quinazolin-2-yl]amino}cyclohexyl)methyl]thiourea | | |
| 782 | N-(2,4-dichloro-6-methylphenyl)-N′-[(cis-4-{[4-(dimethylamino) | 517 (M + H) | 2 |
| | quinazolin-2-yl]amino}cyclohexyl)methyl]thiourea | | |
| 783 | N-[(cis-4-{[4-(dimethylamino)quinazolin-2- | 463 (M + H) | 2 |
| | yl]amino}cyclohexyl)methyl]-N′-(2,5-dimethylphenyl)thiourea | | |
| 784 | N-(2-bromo-4-isopropylphenyl)-N′-[(cis-4-{[4-(dimethylamino) | 555 (M + H) | 3 |
| | quinazolin-2-yl]amino}cyclohexyl)methyl]thiourea | | |
| 785 | N-(2-bromo-5-fluorophenyl)-N′-[(cis-4-{[4-(dimethylamino)- | 531 (M + H) | 3 |
| | quinazolin-2-yl]amino}cyclohexyl)methyl]thiourea | | |
| 786 | N-[(cis-4-{[4-(dimethylamino)quinazolin-2- | 479 (M + H) | 2 |
| | yl]amino}cyclohexyl)methyl]-N′-(2-ethoxyphenyl)thiourea | | |
| 787 | N-[(cis-4-{[4-(dimethylamino)quinazolin-2-yl]amino}- | 491 (M + H) | 1 |
| | cyclohexyl)methyl]-N′-(2-isopropyl-6-methylphenyl)thiourea | | |
| 788 | N-[(cis-4-{[4-(dimethylamino)quinazolin-2- | 479 (M + H) | 3 |
| | yl]amino}cyclohexyl)methyl]-N′-(2-methoxybenzyl)thiourea | | |
| 789 | N-(2,3-dihydro-1,4-benzodioxin-6-yl)-N′-[(cis-4-{[4-(dimethylamino) | 493 (M + H) | 3 |
| | quinazolin-2-yl]amino}cyclohexyl)methyl]thiourea | | |
| 790 | N-[(cis-4-{[4-(dimethylamino)quinazolin-2- | 463 (M + H) | 3 |
| | yl]amino}cyclohexyl)methyl]-N′-(3,4-dimethylphenyl)thiourea | | |
| 791 | N-1,3-benzodioxol-5-yl-N′-[(cis-4-{[4-(dimethylamino)- | 479 (M + H) | 3 |
| | quinazolin-2-yl]amino}cyclohexyl)methyl]thiourea | | |
| 792 | N-(3-chloro-2-methylphenyl)-N′-[(cis-4-{[4-(dimethylamino)- | 483 (M + H) | 3 |
| | quinazolin-2-yl]amino}cyclohexyl)methyl]thiourea | | |
| 793 | N-[4-bromo-2-(trifluoromethoxy)phenyl]-N′-[(cis-4-{[4- | 597 (M + H) | 2 |
| | (dimethyl-amino)quinazolin-2-yl]amino}cyclohexyl)methyl]- | | |
| | thiourea | | |
| 794 | N-(4-chloro-2,5-dimethoxyphenyl)-N′-[(cis-4-{[4-(dimethylamino) | 529 (M + H) | 3 |
| | quinazolin-2-yl]amino}cyclohexyl)methyl]thiourea | | |
| 795 | N-[(cis-4-{[4-(dimethylamino)quinazolin-2- | 491 (M + H) | 3 |
| | yl]amino}cyclohexyl)methyl]-N′-(4-phenylbutyl)thiourea | | |
| 796 | N-(4-tert-butylphenyl)-N′-[(cis-4-{[4-(dimethylamino)quinazolin- | 491 (M + H) | 3 |
| | 2-yl]amino}cyclohexyl)methyl]thiourea | | |
| 797 | N-(5-choloro-2-fluorophenyl)-N′-[(cis-4-{[4-(dimethylamino)- | 487 (M + H) | 3 |
| | quinazolin-2-yl]amino}cyclohexyl)methyl]thiourea | | |
| 798 | N-bicyclo[2.2.1]hept-2-yl-N′-[(cis-4-{[4-(dimethylamino)- | 453 (M + H) | 2 |
| | quinazolin-2-yl]amino}cyclohexyl)methyl]thiourea | | |
| 799 | N-bicyclo[2.2.1]hept-5-en-2-yl-N′-[(cis-4-{[4-(dimethylamino) | 451 (M + H) | 2 |
| | quinazolin-2-yl]amino}cyclohexyl)methyl]thiourea | | |
| 800 | N-(cyclopropylmethyl)-N′-[(cis-4-{[4-(dimethylamino)- | 413 (M + H) | 2 |
| | quinazolin-2-yl]amino}cyclohexyl)methyl]thiourea | | |
| 801 | ethyl 2-[({[(cis-4-{[4-(dimethylamino)quinazolin-2- | 567 (M + H) | 3 |
| | yl]amino}cyclohexyl)methyl]amino}carbonothioyl)amino]- | | |
| | 4.5.6.7-tetrahydro-1-benzothiophene-3-carboxylate | | |
| 802 | methyl 3-[({[(cis-4-{[4-(dimethylamino)quinazolin-2- | 499 (M + H) | 3 |
| | yl]amino}cyclohexyl)methyl]amino}carbonothioyl)amino]- | | |
| | thiophene-2-carboxylate | | |
| 803 | N-(2-bromo-4-fluorophenyl)-N′-[(cis-4-{[4-(dimethylamino)- | 531 (M + H) | 3 |
| | quinazolin-2-yl]amino}cyclohexyl)methyl]thiourea | | |
| 804 | N-(3-chloro-4-fluorophenyl)-N′-[(cis-4-{[4-(dimethylamino)- | 487 (M + H) | 3 |
| | quinazolin-2-yl]amino}cyclohexyl)methyl]thiourea | | |
| 805 | N-(4-butyl-2-methylphenyl)-N′-[(cis-4-{[4-(dimethylamino)- | 505 (M + H) | 3 |
| | quinazolin-2-yl]amino}cyclohexyl)methyl]thiourea | | |
| 806 | N-[4-(dimethylamino)phenyl]-N′-[(cis-4-{[4-(dimethylamino)- | 478 (M + H) | 3 |
| | quinazolin-2-yl]amino}cyclohexyl)methyl]thiourea | | |
| 807 | N-[3-(dimethylamino)propyl]-N′-[(cis-4-{[4-(dimethylamino)- | 472 (M + H) | 3 |
| | quinazolin-2-yl]amino}cyclohexyl)methyl]thiourea | | |
| 808 | N-[(cis-4-{[4-(dimethylamino)quinazolin-2- | 472 (M + H) | 3 |
| | yl]amino}cyclohexyl)methyl]-N′-(2-morpholin-4-ylethyl)thiourea | | |
| 809 | N-[(cis-4-{[4-(dimethylamino)quinazolin-2- | 436 (M + H) | 3 |
| | yl]amino}cyclohexyl)methyl]-N′-pyridin-3-ylthiourea | | |
| 810 | N-[(cis-4-{[4-(dimethylamino)quinazolin-2-yl]amino}- | 486 (M + H) | 3 |
| | cyclohexyl)methyl]-N′-(3-morpholin-4-ylpropyl)thiourea | | |
| 811 | N-[4-(diethylamino)phenyl]-N′-[(cis-4-{[4-(dimethylamino)- | 506 (M + H) | 3 |
| | quinazolin-2-yl]amino}cyclohexyl)methyl]thiourea | | |
| 812 | N-[(cis-4-{[4-(dimethylamino)quinazolin-2-yl]amino}- | 539 (M + H) | 3 |
| | cyclohexyl)methyl]-N′-{4-[(E)-phenyldiazenyl]phenyl}thiourea | | |
| 813 | N-[(cis-4-{[4-(dimethylamino)quinazolin-2- | 470 (M + H) | 3 |
| | yl]amino}cyclohexyl)methyl]-N′-(2-piperidin-1-ylethyl)thiourea | | |
| 814 | N-[(cis-4-{[4-(dimethylamino)quinazolin-2-yl]amino}- | 501 (M + H) | 3 |
| | cyclohexyl)methyl]-N′-[4-(1H-pyrazol-1-yl)phenyl]thiourea | | |
| 815 | N-2,1,3-benzothiadiazol-4-yl-N′-[(cis-4-{[4-(dimethylamino)- | 493 (M + H) | 3 |
| | quinazolin-2-yl]amino}cyclohexyl)methyl]thiourea | | |
| 816 | N-2,1,3-benzothiadiazol-5-yl-N′-[(cis-4-{[4-(dimethylamino)- | 493 (M + H) | 3 |
| | quinazolin-2-yl]amino}cyclohexyl)methyl]thiourea | | |
| 817 | N-[(cis-4-{[4-(dimethylamino)quinazolin-2-yl]amino}- | 454 (M + H) | 3 |
| | cyclohexyl)methyl]-N′-(3,5-dimethylisoxazol-4-yl)thiourea | | |
| 818 | N-[(cis-4-{[4-(dimethylamino)quinazolin-2-yl]amino}- | 502 (M + H) | 3 |
| | cyclohexyl)methyl]-N′-[4-(1,3-oxazol-5-yl)phenyl]thiourea | | |
| 819 | N-[(cis-4-{[4-(dimethylamino)quinazolin-2-yl]amino}- | 516 (M + H) | 2 |
| | cyclohexyl)methyl]-N′-(5-methyl-3-phenylisoxazol-4-yl)- | | |
| | thiourea | | |
| 820 | N-[(cis-4-{[4-(dimethylamino)quinazolin-2-yl]amino}- | 521 (M + H) | 3 |
| | cyclohexyl)methyl]-N′-(6-morpholin-4-ylpyridin-3-yl)thiourea | | |
| 821 | N-[(cis-4-{[4-(dimethylamino)quinazolin-2- | 528 (M + H) | 3 |
| | yl]amino}cyclohexyl)methyl]-N′-(6-phenoxypyridin-3-yl)thiourea | | |
| 822 | N-(cis-4-{[4-(dimethylamino)quinazolin-2-yl]amino}cyclohexyl)- | 468 (M + H) | 2 |
| | N′-(4-fluoro-2-nitrophenyl)urea | | |
| 823 | N-(cis-4-{[4-(dimethylamino)quinazolin-2-yl]amino}cyclohexyl)- | 491 (M + H) | 3 |
| | N′-[4-fluoro-2-(trifluoromethyl)phenyl]urea | | |
| 824 | N-(cis-4-{[4-(dimethylamino)quinazolin-2-yl]amino}cyclohexyl)- | 437 (M + H) | 1 |
| | N′-(4-fluorobenzyl)urea | | |
| 825 | N-(cis-4-{[4-(dimethylamino)quinazolin-2-yl]amino}cyclohexyl)- | 519 (M + H) | 3 |
| | N′-[4-(heptyloxy)phenyl]urea | | |
| 826 | N-(cis-4-{[4-(dimethylamino)quinazolin-2-yl]amino}cyclohexyl)- | 531 (M + H) | 2 |
| | N′-(4-iodophenyl)urea | | |
| 827 | N-(cis-4-{[4-(dimethylamino)quinazolin-2-yl]amino}cyclohexyl)- | 449 (M + H) | 1 |
| | N′-(4-methoxy-2-methylphenyl)urea | | |
| 828 | N-(cis-4-{[4-(dimethylamino)quinazolin-2-yl]amino}cyclohexyl)- | 480 (M + H) | 3 |
| | N′-(4-methoxy-2-nitrophenyl)urea | | |
| 829 | N-(cis-4-{[4-(dimethylamino)quinazolin-2-yl]amino}cyclohexyl)- | 464 (M + H) | 3 |
| | N′-(4-methyl-3-nitrophenyl)urea | | |
| 830 | N-(cis-4-{[4-(dimethylamino)quinazolin-2-yl]amino}cyclohexyl)- | 433 (M + H) | 2 |
| | N′-(4-methylbenzyl)urea | | |
| 831 | N-(4-butoxyphenyl)-N′-(cis-4-{[4-(dimethylamino)quinazolin-2- | 477 (M + H) | 3 |
| | yl]amino}cyclohexyl)urea | | |
| 832 | N-(4-butylphenyl)-N′-(cis-4-{[4-(dimethylamino)quinazolin-2- | 461 (M + H) | 3 |
| | yl]amino}cyclohexyl)urea | | |
| 833 | N-biphenyl-4-yl-N′-(cis-4-{[4-(dimethylamino)quinazolin-2- | 481 (M + H) | 3 |
| | yl]amino}cyclohexyl)urea | | |
| 834 | N-(5-chloro-2,4-dimethoxyphenyl)-N′-(cis-4-{[4- | 499 (M + H) | 1 |
| | (dimethylamino)quinazolin-2-yl]amino}cyclohexyl)urea | | |
| 835 | N-(5-chloro-2-methoxyphenyl)-N′-(cis-4-{[4- | 469 (M + H) | 3 |
| | (dimethylamino)quinazolin-2-yl]amino}cyclohexyl)urea | | |
| 836 | N-(5-chloro-2-methylphenyl)-N′-(cis-4-{[4- | 453 (M + H) | 3 |
| | (dimethylamino)quinazolin-2-yl]amino}cyclohexyl)urea | | |
| 837 | N-(5-chloro-2-nitrophenyl)-N′-(cis-4-{[4- | 484 (M + H) | 3 |
| | (dimethylamino)quinazolin-2-yl]amino}cyclohexyl)urea | | |
| 838 | N-(cis-4-{[4-(dimethylamino)quinazolin-2-yl]amino}cyclohexyl)- | 437 (M + H) | 2 |
| | N′-(5-fluoro-2-methylphenyl)urea | | |
| 839 | N-(2,3-dihydro-1H-inden-5-yl)-N′-(cis-4-{[4- | 445 (M + H) | 3 |
| | (dimethylamino)quinazolin-2-yl]amino}cyclohexyl)urea | | |
| 840 | N-(cis-4-{[4-(dimethylamino)quinazolin-2-yl]amino}cyclohexyl)- | 493 (M + H) | 3 |
| | N′-9H-fluoren-9-ylurea | | |
| 841 | N-(cis-4-{[4-(dimethylamino)quinazolin-2-yl]amino}cyclonexyl)- | 493 (M + H) | 2 |
| | N′-9H-fluoren-9-ylurea | | |
| 842 | N-(cis-4-{[4-(dimethylamino)quinazolin-2-yl]amino}cyclonexyl)- | 433 (M + H) | 2 |
| | N′-(2-phenylethyl)urea | | |
| 843 | N-cyclophenyl-N′-(cis-4-{[4-(dimethylamino)quinazolin-2- | 397 (M + H) | 2 |
| | yl]amino}cyclohexyl)urea | | |
| 844 | N-(cis-4-{[4-(dimethylamino)quinazolin-2-yl]amino}cyclohexyl)- | 495 (M + H) | 1 |
| | N′-(diphenylmethyl)urea | | |
| 845 | methyl 4-({[(cis-4-{[4-(dimethylamino)quinazolin-2- | 463 (M + H) | 3 |
| | yl]amino}cyclohexyl)amino]carbonyl}amino)benzoate | | |
| 846 | 2-(benzyloxy)ethyl (cis-4-{[4-(dimethylamino)quinazolin-2- | 464 (M + H) | 2 |
| | yl]amino}cyclohexyl)carbamate | | |
| 847 | 2,2-dimethylpropyl (cis-4-{[4-(dimethylamino)quinazolin-2- | 400 (M + H) | 3 |
| | yl]amino}cyclohexyl)carbamate | | |
| 848 | 4,5-dimethoxy-2-nitrobenzyl (cis-4-{[4- | 525 (M + H) | 1 |
| | (dimethylamino)quinazolin-2-yl]amino}cyclohexyl)carbamate | | |
| 849 | 3-(trifluoromethyl)phenyl (cis-4-{[4-(dimethylamino)quinazolin- | 474 (M + H) | 3 |
| | 2-yl]amino}cyclohexyl)carbamate | | |
| 850 | 4-bromophenyl (cis-4-{[4-(dimethylamino)quinazolin-2- | 484 (M + H) | 3 |
| | yl]amino}cyclohexyl)carbamate | | |
| 851 | 2-methoxyphenyl (cis-4-{[4-(dimethylamino)quinazolin-2- | 436 (M + H) | 3 |
| | yl]amino}cyclohexyl)carbamate | | |
| 852 | 2-methoxyethyl (cis-4-{[4-(dimethylamino)quinazolin-2- | 388 (M + H) | 3 |
| | yl]amino}cyclohexyl)carbamate | | |
| 853 | octyl (cis-4-{[4-(dimethylamino)quinazolin-2- | 442 (M + H) | 3 |
| | yl]amino}cyclohexyl)carbamate | | |
| 854 | ethyl (cis-4-{[4-(dimethylamino)quinazolin-2- | 358 (M + H) | 3 |
| | yl]amino}cyclohexyl)carbamate | | |
| 855 | 4-nitrobenzyl (cis-4-{[4-(dimethylamino)quinazolin-2- | 465 (M + H) | 1 |
| | yl]amino}cyclohexyl)carbamate | | |
| 856 | 2-naphthyl (cis-4-{[4-(dimethylamino)quinazolin-2- | 456 (M + H) | 3 |
| | yl]amino}cyclohexyl)carbamate | | |
| 857 | allyl (cis-4-{[4-(dimethylamino)quinazolin-2- | 370 (M + H) | 3 |
| | yl]amino}cyclohexyl)carbamate | | |
| 858 | benzyl (cis-4-{[4-(dimethylamino)quinazolin-2- | 420 (M + H) | 2 |
| | yl]amino}cyclohexyl)carbamate | | |
| 859 | phenyl (cis-4-{[4-(dimethylamino)quinazolin-2- | 406 (M + H) | 3 |
| | yl]amino}cyclohexyl)carbamate | | |
| 860 | (1R,2S,5R)-2-isopropyl-5-methylcyclohexyl (cis-4-{[4- | 468 (M + H) | 3 |
| | (dimethylamino)quinazolin-2-yl]amino}cyclohexyl)carbamate | | |
| 861 | 4-methylphenyl (cis-4-{[4-(dimethylamino)quinazolin-2- | 420 (M + H) | 3 |
| | yl]amino}cyclohexyl)carbamate | | |
| 862 | methyl (cis-4-{[4-(dimethylamino)quinazolin-2- | 344 (M + H) | 3 |
| | yl]amino}cyclohexyl)carbamate | | |
| 863 | 2-chlorobenzyl (cis-4-{[4-(dimethylamino)quinazolin-2- | 454 (M + H) | 2 |
| | yl]amino}cyclohexyl)carbamate | | |
| 864 | 9H-fluoren-9-ylmethyl (cis-4-{[4-(dimethylamino)quinazolin-2- | 508 (M + H) | 3 |
| | yl]amino}cyclohexyl)carbamate | | |
| 865 | 2,2,2-trichloroethyl (cis-4-{[4-(dimethylamino)quinazolin-2- | 460 (M + H) | 3 |
| | yl]amino}cyclohexyl)carbamate | | |
| 866 | 2-(benzyloxy)ethyl [(cis-4-{[4-(dimethylamino)quinazolin-2- | 478 (M + H) | 3 |
| | yl]amino}cyclohexyl)methyl]carbamate | | |
| 867 | 2,2-dimethylpropyl [(cis-4-{[4-(dimethylamino)quinazolin-2- | 414 (M + H) | 3 |
| | yl]amino}cyclohexyl)methyl]carbamate | | |
| 868 | 4,5-dimethoxy-2-nitrobenzyl [(cis-4-{[4-(dimethylamino)- | 539 (M + H) | 3 |
| | quinazolin-2-yl]amino}cyclohexyl)methyl]carbamate | | |
| 869 | 3-(trifluoromethyl)phenyl [(cis-4-{[4-(dimethylamino)quinazolin- | 488 (M + H) | 3 |
| | 2-yl]amino}cyclohexyl)methyl]carbamate | | |
| 870 | 4-bromophenyl [(cis-4-{[4-(dimethylamino)quinazolin-2- | 498 (M + H) | 3 |
| | yl]amino}cyclohexyl)methyl]carbamate | | |
| 871 | 2-methoxyphenyl [(cis-4-{[4-(dimethylamino)quinazolin-2- | 450 (M + H) | 3 |
| | yl]amino}cyclohexyl)methyl]carbamate | | |
| 872 | 2-methoxyethyl [(cis-4-{[4-(dimethylamino)quinazolin-2- | 402 (M + H) | 3 |
| | yl]amino}cyclohexyl)methyl]carbamate | | |
| 873 | octyl [(cis-4-{[4-(dimethylamino)quinazolin-2- | 456 (M + H) | 3 |
| | yl]amino}cyclohexyl)methyl]carbamate | | |
| 874 | ethyl [(cis-4-{[4-(dimethylamino)quinazolin-2- | 372 (M + H) | 3 |
| | yl]amino}cyclohexyl)methyl]carbamate | | |
| 875 | 4-nitrobenzyl [(cis-4-{[4-(dimethylamino)quinazolin-2- | 479 (M + H) | 2 |
| | yl]amino}cyclohexyl)methyl]carbamate | | |
| 876 | 2-naphthyl [(cis-4-{[4-(dimethylamino)quinazolin-2- | 470 (M + H) | 3 |
| | yl]amino}cyclohexyl)methyl]carbamate | | |
| 877 | allyl [(cis-4-{[4-(dimethylamino)quinazolin-2- | 384 (M + H) | 3 |
| | yl]amino}cyclohexyl)methyl]carbamate | | |
| 878 | benzyl [(cis-4-{[4-(dimethylamino)quinazolin-2- | 434 (M + H) | 2 |
| | yl]amino}cyclohexyl)methyl]carbamate | | |
| 879 | phenyl [(cis-4-{[4-(dimethylamino)quinazolin-2- | 420 (M + H) | 3 |
| | yl]amino}cyclohexyl)methyl]carbamate | | |
| 880 | (1R,2S,5R)-2-isopropyl-5-methylcyclohexyl [(cis-4-{[4- | 482 (M + H) | 3 |
| | (dimethyl-amino)quinazolin-2-yl]amino}cyclohexyl)methyl] | | |
| | carbamate | | |
| 881 | 4-methylphenyl [(cis-4-{[4-(dimethylamino)quinazolin-2- | 434 (M + H) | 3 |
| | yl]amino}cyclohexyl)methyl]carbamate | | |
| 882 | methyl [(cis-4-{[4-(dimethylamino)quinazolin-2- | 358 (M + H) | 3 |
| | yl]amino}cyclohexyl)methyl]carbamate | | |
| 883 | 2-chlorobenzyl [(cis-4-{[4-(dimethylamino)quinazolin-2- | 468 (M + H) | 2 |
| | yl]amino}cyclohexyl)methyl]carbamate | | |
| 884 | 9H-fluoren-9-ylmethyl [(cis-4-{[4-(dimethylamino)quinazolin-2- | 522 (M + H) | 3 |
| | yl]amino}cyclohexyl)methyl]carbamate | | |
| 885 | 2,2,2-trichloroethyl [(cis-4-{[4-(dimethylamino)quinazolin-2- | 474 (M + H) | 3 |
| | yl]amino}cyclohexyl)methyl]carbamate | | |

### Example 886

### N-(cis-4-{[4-(dimethylamino)-6-methylquinazolin-2-yl]amino}cyclohexyl)-2,2-diphenylacetam idetrifluoroacetate

### S Step A: Synthesis of 6-methyl-1H-quinazoline-2,4-dione

To a suspension of 2-amino-5-methylbenzoic acid (5.27 g, 0.035 mol) in 150 mL H₂O and 2 mL acetic acid was added potassium cyanate (3.67 g, 0.045 mol) predissolved in 30 mL H₂O. The reaction mixture was stirred for 5 hours and then 10 g NaOH pellets were added with continued stirring. The mixture was cooled to 0 ° C. in an ice bath and another 30 g, NaOH pellets were added. During the addition of NaOH a precipitate was formed. This precipitate was filtered and resuspended in 100 mL H₂O and 3M HCl was added by pipette until the aqueous solution was slightly acidic. The precipitate was then filtered and washed with ice cold H₂O to yield 6-methyl-1 H-quinazoluie-2,4-dione (2.2 g, 37%) as an off white solid. ESI-MS m/e 177.1 M+H⁺; ¹H NMR (400 MHz, DMSO-d₆) δ 11.18 (s, 1H) 11.02 (s. 1 H), 7.66 (s, 1 H), 7.45 (d, J=8.4 Hz, 1 H), 7.05 (d, J=8.4 Hz, 1 H), 2.31 (s, 3H).

### Step B: Synthesis of 2,4-dichloro-6-methyl-quinazoline

To a solution of 6-methyl-1 H-quinazoline-2.4-dione (2.29 g, 0.013 mol) in 20 mL POCl₃ was added N,N-dimethylaniline (1.81 mL, 0.014 mol). The mixture was heated to reflux (125 ° C.) and stirred for 4 hours until the starting material completely dissolved and the solution turned dark purple in color. The solution was then cooled and poured slowyl) on ice (40 g; catition highly exothennic) to quench the reaction. The aqueous layer was then extracted three times with CH₂Cl₂ (40 mL). The organic layer was dried over MgSO₄, concentrated, and subjected to purification b)y chromatography (100% CH₂Cl₂) to yield 2,4-dichloro-6-methyl-quinazolin (2.5 g,90 %) as a slightly yellow, solid. ¹ H NMR (400 MHz, DMSO-d₆) δ 8.05 (s, 1H), 8.01 (d, J=9.2 Hz, 1 H), 7.94 (d, J=8.8 Hz, 1 H), 2.57 (s. 3 H).

### Step C: Synthesis of (2-chloro-6-methyl-quinazolin-4-yl)-dimethyl-amine

A solution of 2,4-dichloro-6-methyl-quinazoline (2.5 g, 0.012 mol) in CH₂Cl₂ (100 mL) was cooled on an ice bath with stirring. Dimethylamine (23.6 mL, 0.047 mol) was added slowly to the solution removed from the ice bath. The. mixture stirred for 1 hour and the excess solvents were evaporated. The compound was subject to purification by chromatography (100% CH₂Cl₂) to yield (2-chloro-6-methyl-quinazolin-4-yl)-dimethy-amine (2.4 g, 92%) as a white solid. ESI-MS m/e 222.2 M+H⁺; ¹H NMR (400 MHz, DMSO-d₆), δ 7.06 (s, 1 H), 7.61 (d, J=8 Hz, 1 H). 7.54 (d, J=8.4 Hz, 1 H), 3.34 (brs. 6 H), 2.45 (s, 3 H).

### Step D: Synthesis of cis-[4-(4-dimethylamino-6-methyl-quinazolin-2-ylamino)-cyclohexyl]-carbamic acid tert-butyl ester

To a solution of (2-chloro-6-methyl-quinazolin-4-yl)-dimethyl-amine (0.5 g, 0.0023 mol) in 0.5 mL 2-propanol was added of cis-(4-amino-cyclohexyl)-carbamic acid tert-butyl ester (483 mg, 0.0023 mol), and DIEA (786 uL, 0.0045 mol). The reaction mixture was heated in a microwave synthesizer at 170° C for 1 hour. The solvent was evaporated and the material subjected to chromatography (2-4% 2M NH₃ in MeOH/CH₂Cl₂) to yield cis-c4-(4-dimethylamino-6-methyl-quiazolin-2-ylamino)-cyclohexy]-carbamic acid tert-butyl ester (850 mg, 94%) as a white solid.

ESI-MS m/e 400.4 M+H⁺; ¹H NMR (400 MHz, CD₃OD) δ 7.68 (s, 1 H)7.37 (d, J=8.4 Hz, 1 H), 7.28 (d, J=8.4 Hz, 1 H), 4.05 (m, 1 H), 3.54 (brs, 1 H), 3.26 (s, 6 H), 2.38 (s, 3 H), 1.76-1.59 (m, 8 H), 1.44 (s, 9 H).

### Step E: Synthesis of cis-N²-(4-amino-cyclohexyl)-6,N⁴,N⁴-trimethyl-quinazoline-2,3-diamine

To a solution of cis-[4-(4-dimethylamino-6-methyl-quinazolin-2-ylaminno)-cyclohexyl]-carbamic acid tert-butyl ester (850 mg, 0.0021 mol) in 30 mL CH₂Cl₂ was added TFA (325 uL, 0.042 mol). The solution was stirred at room temperature for 4 hours. The excess solvent was evaporated offand the resulting oil was dissolved in 30 mL CH₂Cl₂. The organic layer was extracted with 30 mL of a dilute NaOH (aq)/NaHCO₃ (aq) solution. The aqueous layer was back extracted twice with CH₂Cl, and the organic layers combined, dried over MgSO₄, and concentrated to yield cis-N²-(4-amino-cyclohexyl)-6,N⁴,N⁴-trimethyl-quinazoline-2,4-diamine (459 mg, 72%) as a white solid.

ESI-MS m/e 300.2 M+H⁺; ¹H NMR (400 MHz, CD₃OD) δ 7.69 (s, 1 H), 7.38 (d, J=8.4 Hz, 1 H), 7.30 (d, J=8.8 Hz, 1 H), 4.07 (m, 1 H), 3.27 (s, 6 H), 2.S5 (mi, 1 H), 2.39 (s, 3 H), 1.84-1.70 (m, 6 H), 1.57-1.52 (m, 2H).

### Step F: Synthesis of N-(cis-4-{[4-(dimethylamino)-6-methylquinazolin-2-6l]amino}-cyclohexyl)-2,2-diphenylacetamide trifluoroacetate

To a solution of cis-N²-(4-amino-cyclohexyl)-6,N⁴,N⁴-trimethyl-quinazoline-2,4-diamine (24.9 mg, 0.083 mmol) in 0.5 mL DMF was added pyridine (16.2 uL, 0.2 mmol) and diphenylacetyl chloride (23.0 mg, 0.1 mmol). The reaction mixture was stirred overnight and then 0.5 mL of DMSO was added to the mixture. The compound was then subject to purification by prep LCMS to yield N-(cis-4-{[4-(dimetylamino)-6-methylquinazolin-2-yl]amino}cyclohexyl)-2,2-diphenylacetamide trifluoroacetate (13.6 mg, 27%) as a white solid.

ESI-MS m/e 494.4 M+H⁺; ¹ H NMR (400 MHz, CD₃OD) δ 7.96 (s, 1 H), 7.63 (d, J=8.4 Hz, 1 H), 7.31-7.23 (m, 11 H), 4.16 (brs, 1 H), 3.89 (brs, 1 H), 3.54 (brs, 6 H), 2.66 (s, 1 H), 2.47 (s, 3 H), 1.86-1.79 (m, 8 H).

### Example 887

### N-(cis-4-{[4-(Dimethylamino)-6-methylquinazolin-2-6l]amino}cyclohexyl)4-fluoro-3-(trifluoromethyl)benzamide trifluoroacetate

### Step A: Synthesis of N-(cis-4-{[4-(dimethylamino)-6-methylquinazolin-2-6l]amino}-cyclohexyl)-4-fluoro-3-(trifluoromethyl)benzamide trifluoroacetate

Using a similar procedure as described in step F of Example 386, the title compound was obtained (12.5 mg, 25%) as a white solid.

ESI-MS m/e 490.2 M+H⁺; ¹H NMR (400 MHz, CD₃OD) δ 8.12-8.15 (m, 2 H), 7.98 (s, 1 H), 7.64 (d, J=8.4 Hz, 1 H), 7.49 (t, J=9.2 Hz, 1 H), 7.44 (brs, 1 H), 4.24 (brs. 1 H), 4.03 (brs, 1 H), 3.56 (s, 6H), 2.47(s, 3H),2.01-1.81 (m, 8H).

### Example 888

### N-(cis-4-{[4l-(Dimethylamino)-6-methylquinazolin-2-yl]amino}cyclohexyl)3,5-bis(trifluoromethyl)benzamide trifluoroacetate

### Step A: Synthesis of N-(cis-4-{[4-(dimethylamino)-6-methylquinazolin-2-yl]amino}-cyclohexyl)-3,5-bis(trifluorometbyl)benzamide trifluoroacetate

Using a similar procedure as described in step F of Example 886, the title compound was obtained (18.4 mg, 0.028 mmol, 34%) as a white solid.

ESI-MS m/e 540.4 M+H⁺; ¹H NMR (400 MHz, CD₃OD) δ 8.53 (s, 2 H), 8.18 (s, 1 H), 7.97 (s, 1 H), 7.64 (d, J:=8.4 Hz, 1 H), 7.37 (brs, 1 H), 4.26 (brs, 1 H), 4.07 (brs, 1 H), 3.56 (brs, 6 H), 2.47 (s, 3 H), 2.07-1.32 (m, 8 H).

### Example 889

### N-(cis-4-{[4-(Dimethylamino)-6-methylquinazolin-2-yl]amino}cyclohexyl)-3,4,5-trimethoxybenzamide trifluoroacetate

Using a similar procedure as described in step F of Example 886, the title compound was obtained (21.2 mg, 0.035 mmol, 42%) as a white solid.

ESI-MS m/e 494.4 M+H⁺; ¹H NMR (400 MHz, CD₃OD) δ 7.98 (s, 1 H), 7.64 (d, J=8.4 Hz, 1 H), 7.37 (brs, 1 H), 7.17 (s, 2 H), 4.28 (brs, 1 H), 4.02 (brs, 1 H), 3.91 (s, 6 H), 3.82 (s, 3 H) 3. (brs, 6 H), 2.47 (s, 3 H), 2.07-1.81 (m, 8 H).

### Example 890

### cis-4-{[4-(Dimethylamino)-6,7-difluoroquinazolin-2yl]amino}-[4-(4-methylbenzyl)cyclohexane carboxamide trifluoroacetate

### Step A: Synthesis of 6,7-difluoro-1H-quinazoline-2,4-dione

A solution of KOCN (6.1 g, 75 mmol) in H₂O (52 mL.) was added to a solution of 2-amino-4,5-difluoro benzoic acid (10 g, 58 mmol) in H₂O/AcOH (260 mL/3.5 mL). The mixture was stirred overnight at room temperature, and then NaOH (55 g, 1.37 mol) was slowly added in a portion of 3˜4 grams. During the addition of NaOH, the reaction was changed to a clear purple solution, and then formation of precipitates was observed. After stirring about 10 min, the precipitates wvere filtered and resuspended in H₂O. The aqueous suspension was acidified to pH 4 with 4N-HCl and stirred for another 10 more min. The precipitates were filtered and washed with cold water and dried to give 7.0 g (61%) of 6,7-difluoro-1H-quinazoline-2,4-dione.

ESI MS m/e 199 M+H⁺; ¹H NMR (400 MHz, DMSO-d₆) δ 11.46 (s, 1 H), 11.26 (s, 1 H), 7.81 (dd, J=10.0, 8.4 Hz 1 H), 7.08 (dd, J=11.2, 6.8 Hz, 1 H).

### Step B: Synthesis of 2,4-dichloro-6,7-difluoroquinazoline

To a suspension of 6,7-difluoro-1H-quinazoline-2,4-dione (6.9 g, 5 mmol) in POCl₃ (21 mL) was slowlyl added N,N-dimethylaniline (4.9 mL, 3 mol). The reaction was heated at reflux (120° C. for 7 h until the starting material was completely dissoled and the entire solution turned a dark purple color. The reaction was allowed to cool and poured veiy slowly onto ice (1 L); watch out for heat generation!! The resulting precipitate was filtered and washed with ice water. The crude product was purified from a short column of silica with CH₂Cl₂ as an elutinc solvent. The desired product (7.2 88%) was obtained as a white solid.

ESI MS m/e 236 M+H⁺; ¹H NMR (400 MHz CDCl₃) δ 8.01 (dd, J=9.2, 8.0 Hz, 1 H), 7.76 (dd, J=10.0, 7.2 Hz, 1 H).

### Step C: Synthesis of 2-chloro-6,7-difluoro-4-dimethylamminoquinazoline

A solution of 2,4-dichloro-6,7-difluoro quinaoline (6.1 g, 26 mmol) in THF (60 mL) was cooled to 2˜4 ° C. in an ice bath and 2M-Me₂NH in MeOH (2 mL, ˜2 eq.) was slowly added. The reaction was stirred for 70 min. at room temperature, neutralized with saturated aqueous NaHCO₃, and concentrated until the most volatile solvent was removed. Addition of water into the concentrated crude reaction mixture gave solid precipitate, which was filtered and dried. 2-Chloro-6,7-difluoro-4-dimethylaminoquinazoline pure compound (5.6 g, 90%) was isolated as a yellowish white solid from a short column of silica using CH₂Cl₂/MeOH (100/0 to 90/10) as an eluting solvent.

ESI MS m/e 244 M+H⁺; ¹H NMR (400 MHz, CDCl₃) o 7.78 (dd, J=11.2, 8.0 Hz, 1 H), 7.50 (dd, J=11.2, 80 Hz, 1 H), 3.40 (s, 6 H).

### Step D: Synthesis of cis-4-(4-dimethylamino-6,7-difuoroquinazolin-2-ylamino)-cyclohexane-carboxylic acid ethyl ester

A suspended solution of 2-chloro-6,7-difluoro-4-dimethylamino quinazoline (0.45 g, 1.85 mmol) and cis-(4-ethoxycarbonyl) aminocyclohexane hydrochloride (0.38 g, 1 eq.) in IPA (2.5 mL) and DIEA (0.5 mL. -2eq.) was reacted for 2 h at 155 ° C. in a Smith microwave synthesizer. The reaction was quenched and purified by column chromatogiaphy (DCM:MeOH=100:0 to 90:10) to give 0.25 g (36%) of cis-4(4-dimethylamino-6,7-difluoroquiazolin-2-ylamino)-cyclohexanecarboxylic acid ethyl ester.

ESI MS m/e 379 M+H⁺; ¹H NMR (400 MHz, CDCl₃) δ 7.57 (dd, J=11.0, 8.0 Hz, 1 H), 7.17 (dd, J=12.0, 7.0 Hz, 1H), 4.96 (d, J=7.0 Hz, 1 H), 4.15 (q, J=7.0 Hz, 2 H), 4.13 (brs, 1 H), 3.23 (s, 6 H), 2.48 (m, 1 H), 1.94 (m, 2 H), 1.83-1.6S (n, 6 H), 1.25 (t, J=7.0 Hz, 3 H).

### Step E: Synthesis of cs-4-(4-dimethylamino-6,7-difluoroquinazolin-2-ylamino)-cyclohexanecarboxylic acid

A suspension of cis-4(4-dimethylamino-6,7-difluoroquinazolin-2-ylamino)-cyclohexane carboxylic acid ethyl ester (0.71 g 1.9 mmol) in 4N-HCl (15 mL) was stirred at 82° C. for 3 h. During the reaction, the heterogenous solution turned to be a clear solution, and then the precipitate was formed. The solid was filtered, washed with cold water several times, and dried to give 0.55 g (85%) of cis-4(4-dimethylamino-6,7-difluoroquinazolin-2-ylamino)-cyclohexane carboxylic acid as a white solid.

ESI MS m/e 351 M+H⁺; ¹H NMR (400 MHz, DMSO-d₆) δ 12.15 (brs, 1 H), 8.18 (m, 2 H), 7.47 (m, 1 H), 3.99 (brs, 1 H), 3.38 (s, 6 H), 2.38 (brs, 1 H), 1.75-1.59 (m, 8 H).

### Step F: Synthesis of cis-4-{[4-(dimethylamino)-6,7-difuoroquinazolin-2-yl]amino}-N-(4-methylbenzyl)cyclohexane carboxamide trifluoroacetate

cis-4(4-Dimethylamino-6,7-difluoroquinazolin-2-ylamino)-cyclohexane carboxylic acid (21 mg, 0.06 mmol) and 4-methylbenzyl amine (7.5 mg, 0.06 mmol) was stirred overnight in the presence of HATU (25 mg, 1.1 eq.) and Et₃N (5 drops). cis-4-{[4-(dimethylamino)-6,7-difluoroquinazolin-2-yl]amino}-N-(4-methylbenzyl)cyclohexanecarboxamide trifluoroacetate (13 mg, 39%) was obtained ftom a prep-HPLC.

ESI MS m/e 454 M+H⁺; ¹H NMR (400 MHz, DMSO-d₆) δ 11.9 (brs, 1 H), 8.19 (m, 2 H), 8.10 (b, 1 H), 7.49 (m, 1 H), 7.05 (s, 4 H), 4.16 (d, J=6.0 Hz, 2 H), 4.08 (brs, 1 H), 3.39 (s, 6 H), 2.26 (m, 1 H), 2.20 (s, 3 H), 1.71-1.57 (m, 8 H).

### Example 891

### cis-N-(3-Chlorobenzyl)-4-{[4-(dimethylamino)-6,7-difluoroquinazolin-2-yl]amino}cyclohexane carboxamide trifluoroacetate

### Step A: Synthesis of cis-N-(3-chlorobenzyl)-4-{[4-dimethylamino)-6,7-difluoroquinazolin-2-yl]amino}cyclohexane carboxamide trifluoroacetate

Using a similar procedure as described in step F of Example 890, the title compound intas obtained.

ESI MS m/e 474 M+H¹; ¹H NMR (400 1MHz, DMSO-d₆)δ 1.21 (brs, 1 H), 8.31 (t,J=7.6 Hz, 1 H), 8.19 (m, 2 H), 7.49 (t, J=8.0 Hz, 1 H), 7.30-7.21 m, 3 H), 7.13 (d, J=7.6 Hz, 1 H), 4.21 (d, J=6.0 Hz, 2 H), 4.08 (brs, 1 H), 3.44 (s, 6 H), 2.29 (brs, 1 H), 1.85-1.59 (m, 8 H),

### Example 892

### cis-4-{[4-(Dimethylamino)-6,7-difluoroquinazolin-2-yl]amino}-N-[(1R)-1-(3-methoxphenyl)ethyl]cyclohexanecarboxamide trifluoroacetate

### Step A: Synthesis of cis-4-{[4-(dimethylamino)-6,7-difluoroquinazolin-2-yl]amino}-N-](1R)-1-(3-methoxyphenyl)ethyl]cyclohexanecarboxamide trifluoroacetate

Using a similar procedure as described in step F of Example 890, the title compound was obtained.

ESI MS m/e 484 M+H⁺; ¹H NMR (400 MHz, DMSO-₆) δ 11.8 (brs, 1 H), 8.19 (m, 1 H), 8.12 (m, J=8.0 Hz, 1 H), 8.07 (brs, 1 H), 7.49 (t, J=8.0 Hz, 1 H), 7.14 (t, J=8.0 Hz, 1 H), 6.80 (d, J=7.6 Hz, 1 H), 6.79 (s, 1H), 6.70 (d, J=7.6 Hz, 1 H), 4.82 (m, 1 H), 4.03 (brs, 1 H), 3.66 (s, 3 H), 3.37 (s, 6 H), 2.26 (brs, 1 H), 1.69-1.52 (m, 8 H), 1.23 (d, J=7.2 Hz, 3 H).

### Example 893

### N-(3,4-Dimethoxyphenyl)-N′-(cis-4-{[4-(dimethylamino)quinazolin-2-yl]amino}-cyclohexyl)urea trifluoroacetate

### Step A: Synthesis of cis-(4-benzylcarbonylamino-cyclohexyl)-carbamic acid tert-butyl ester

To a suspension of cis-4-tert-butoxycarbonylamino-cyclohexane carboxylic acid (50 g, 0.21 mol) in benzene was added triethylamine (37 mL, 0.27 mol) and diphenylphosphoryl azide (48.7 mL, 0.23 mol). The reaction mixture was stirred at 80° C. for 1 hour. Benzyl alcohol (30 mL, 0.29 mol) was added and the reaction mixture was stirred at reflux overnight. The solvent benzene was removed under vacuum and the resulting slurry dissolved in etlhyl acetate. The organic layer was extracted with H₂O and separated. The aqueous layer was extracted twice more with ethyl acetate. The organic layers were combined, dried over MgSO₄, concentrated, and subjected to chromatography (30% ethyl acetate in hexanes) to give cis-(4-benzyloxycarbonylamino-cyclohexyl)-carbamic acid tert-butyl ester (54.1 g, 0.16 mol, 75%) as a colorless oil.

ESI-MS m/e 349.4 M+H⁺; ¹H NMR (400 , MHz, DMSO-d₆) δ 7.34-7.28 (m, 5 H), 7.12 (d. J=5.6 Hz, 1 H), 6.62 (brs, 1 H), 4.98 (s, 2 ), 3.39-3.37 (m, 2 H), 1.60-1.45 (m, 8 H), 1.37 (s, 9 H).

### Step B: Synthesis of cis-(4-amino-cyclohexyl)-carbamic acid tert-butyl ester

To a solution of cis-(4-benzyloxycarbonylamino-cyclohexyl)-carbamic acid tert-butyl ester (54.1 g, 0.16 mol) in ethanol was added 10% Pd/C (5.4 g). The reaction mixture was stirred at room temperature under an H₂ atmosplere for 3 hours. The H₂ atmosphere as removed and the solution filtered though celite and concentrated. The resulting precipitate was dissolved in ethyl acetate and extracted with a dilute NaOH (aq) solution. The aqueous layer as extracted twice more with ethyl acetate. The organic layers were combined, dried over MgSO₄, and concentrated. The resulting precipitate was recrystallized in ethyl acetate and hexanes to yield cis-(4-amino-cyclohexyl)-carbamic acid tert-butyl ester (28.9 g, 0.14 mol, 87%) as a white solid.

ESI-MS m/e 215.2 M+H⁺; ¹H NMR (400 MHz, DMSO-d₆) δ 6.60 (d, J=6.0 Hz, 1 H)3.30-3.28 (m, 1 H), 2.74 (s, 1 H), 1.59-1.51 (m, 2 H), 1.45-1.37 (m, 15 H).

### Step C: Synthesis of cis-[4-(4-dimethlyamino-quinazolin-2-ylamino)-cyclohexyl]-carbamic acid tert-butyl ester

To a solution of cis-(4-amino-cyclohexyl)-carbamic acid tert-butyl ester (0.5 g, 0.0023 mol) in 1 mL 2-propanol was added (2-chloro-quinazolin-4-yl)-dimethyl-amine (0.53, 0.0026 mol) and DIEA (1.22 mL. 0.0070 mol). The mixture was heated in a microwave synthesizer at 170° C. for 1 hour. The reaction was repeated 39 more times (20 g total material) and the reaction mixtures were pooled. The solvent was evaporated and the material subjected to chromatography (2-4% 2M NH₃ in MeOH /CH₂Cl₂) to yield cis-[4-(4-dimethlyamino-quinazolin-2-ylamino)-cyclohexyl]-carbamic acid tert-butyl ester (22.1 , 0.057 mol, 61%) as a colorless oil.

ESI-IS m/e 386.4 M+H⁺; ¹H NMR (400 MHz, DMSO-d₆) δ 7.85 (d, J=8.0 Hz, 1 H), 7.47 (t, J=8.4 Hz, 1 H), 7.27 (d, J=3.0 Hz, 1 H), 7.00 (t, J=7.6 Hz, 1 H), 6.60 (brs, 1 H), 6.18 (brs, 1 H), 3.89-3.88 (m, 1 H), 3.39 (brs, 1 H), 3.19 (s, 6 H), 1.77-1.71 (m, 2 H), 1.68-1.52 (m, 6 H), 1.38 (s, 9 H).

### Step D: Synthesis of cis-N2-(4-amino-cyclohexyl)-N⁴,N⁴-dimethyl-quinazolin-2,4-diamine

To a solution of cis-(4-(4-dimethlyamino-quinazolin-2-ylamino)-cyclohexyl]-carbamic acid tert-butyl ester (22.1 g, 0.057 mol) in CH₂Cl₂ was added TFA (10 mL, 0.13 mol). The solution was stirred at room temperattire for 4 hours. The excess solvent was evaporated off and the resulting oil was dissolved in CH₂Cl₂. The organic layer was extracted with a dilute NaOH (aq)/NaHCO₃ (aq) solution. The aqueous layer was extracted twice more with CH₂Cl₂ and the organic layers combined, dried over MgSO₄, and concentrated. The resulting precipitate was crystallized in ether and hexanes to yield cis-N²-(4-amino-cyclohexyl)-N⁴,N⁴-dimethyl-quinazolin-2,4-diamine (15.0 g, 0.053 mol, 92%) as a pale yellow solid.

ESI-MS m/e 286.2 M+H⁺; ¹H NMR (400 MHz, DMSO-d₆) δ 7.84 (d, J 8.4 Hz, 1 H), 7.45 (t, J=6.8 Hz, 1 H), 7.26 (d, J=8.4 Hz, 1 H), 6.99 (t, J=7.6 Hz, 1 H), 6.20 (brs, 1 H), 3.90-3.89 (m, 1 H), 3.18 (s, 6 H) 2.79 (s, 1 H), 1.74-1.71 (m, 2 H), 1.57-1.41 (m, 8 H).

### Step E: Synthesis of N-(3,4-dimethoxyphenyl)-N′-(cis-4-{[4-(dimethylamino)quinazolin-2-yl]amino}cyclohexyl)urea trifluoroacetate

To a solution of cis-N²(4-aminp-cyclohexyl)-N⁴,N⁴-dimethyl-quinazolin-2,4-diamine (28.5 mg, 0.10 mmol) in 0.5 mL of DMSO was added 3,4-dimethoxyphenylisocyanate (14.9 uL, 0.10 mmol). Note that for this reaction it was necessary to slightly heat the starting material to dissolve it in the DMSO before adding the isocyanate. The reaction mixture was stirred for 1 hour and then 0.5 mL of 50% DMSO in H₂O was added. The compound was subjected to purification by prep LCMS to yield N-(3,4-dimethoxyphenyl)-N′-(cis-4-{[4-(dimethylamino)quinazolin-2-yl]amino}cyclohexyl)urea trifluoroacerate (37 mg, 0.064 mmol, 64%) as a white solid.

ESI-MS m/e 465.2 M+H⁺; ¹H NMR (400 MHz, DMSO-d₆) δ 12.10 (s, 1 H), 8.21 (s, 1 H), 8.16 (d, J=8.0 Hz, 1 H), 8.08 (brs, 1 H), 7.78 (t, J=7.6 Hz, 1 H), 7.45 (brs, 1 H), 7.37 (t, J=7.6 Hz, 1 H), 7.15 (s, 1 H), 6.83-6.72 (m, 2 H), 6.15 (d, J=6.8 Hz, 1 H), 4.00 (brs, 1 H), 3.72 (s,3 H), 3.69 (s, 3 H), 3.47 (brs, 6 H), 1.80-1.78 (m, 2 H), 1.68 (m, 6 H).

### Example 894

### N-[(cis-4-{[4-(Dimethylamino)quinazolin-2-yl]amino}i ) cyclohexyl)methyl]-N′-[2-(trifluoromethoxy)phenyl]urea trifluoroacetate

### Step A: Synthesis of cis-4-tert-butoxycarbonylamino-cyclohexanecarboxylic acid

To a solution of cis-4-amino-cyclohexanecarboxylic acid (50 g, 350 mmol) in 200 mL of THF and 38 0mL of 1M NaOH (380 mmol), Boc₂O (83.5 g, 360 mmol) was added. The mixture was stirred at room temperature for 2 hr and evaporated until only water was remained. The reaction mixture was cooled to 0° C. and acidified with 1M HCl until pH about 3. The white solid formed was filtered, washed with water and hexanes to give cis-4-tert-butoxycarbonylamino-cyclohexanecarboxylic acid (71 g, 83%) as a white solid.

ESI-IS m/e 244 M+H⁺; ¹H NMR (400 MHz, DMSO-d₆) δ 12.00 (b, 1 H), 6.74 (d, J=4.25, 1 H), 3.30 (brs, 1 H), 2.35 (m, 1 H), 1.87 (m, 2 H), 1.55-1.37 (m 15 H).

### Step B: Synthesis of cis (4-carbamoyl-cyclohexyl)-carbamic acid tert-butyl ester

The cis-4-tert-butoxycarbonylamino-cyclohexanecarboxylic acid (68 g, 280 mmol) and triethylamine (42.35 mL, 308 mmol) were dissolved in 300 mL of THF and the mixture was cooled to 0° C. Ethyl chloroformate (29.3 mL, 308 mmol) was added dropwise. After stirring at 0° C. for 30 min, 168 mL of aqueous ammonia was added dropwise. The mixture was allowed to stir at room temperature for 2 hr. The solvent was evaporated until only water was remained. To this mixture was added EtOAc. The organic layer was washed with sat. NaHCO₃, 1M HCl, brine, water, dried over Na₂SO₄ and filtered. The solvent was evaporated to give cis (4-carbamoyl-cycloeexyl)-carbamic acid tert-butyfl ester (62 g, 88%) as a white solid.

ESI-MS m/e 243 M+H⁺; ¹H NMR (400 MHz, DMSO-d₆) δ 7.10 (brs, 1 H), 6.69 (bis, 2 H), 3.41 (brs, 1 H), 2.14 (m, 1 H), 1.79 (m, 2 H), 1.59 (m, 2 H), 1.45-1.37 (m, 13 H).

### Step C: Synthesis of cis-4-amino-cyclohexanecarboxylic acid amide hydrochloride

The cis-(4-carbamoyl-cyclohexyl)-carbamic acid tert-butyl ester (62 g, 256 mmol) in 250 mL of DMC was added 250 mL of TFA. The mixture was stirred for 1 hr. The solvents wvere evaporated. To the residue was added 150 mL of 2M HCl in ether to give white solid. The solvent was evaporated to give cis-4-amino-cyclohexanecarboxylic acid amide hydrochloride (45 g, 98%) of white solid as the product.

ESI-MS m/e 143 M+H⁺; ¹H NMR (400 MHz, DMSO-d₆) δ 8.08 (brs, 3 H), 7.28 (s, 1 H), 6.78 (s, 1 H), 3.10(m, 1 H), 2.24 (m 1 H), 1.90 (m, 2 H), 1.66 (m, 4 H), 1.50 (m, 2 H).

### Step D: Synthesis of cis-4-)4-dimethylamino-quinazolin-2-ylamino)-cyclohexanecarboxylic acid amide

(2-Chloro-quinazoline-4-yl)-dimethylamine (31.05 g, 150 mmol) and cis-4-amino-cyclohexanecarboxylic acid amide hydrochloride (26.7 g, 150 mmol) in 150 mL of pyridine was refluxed overnight. The solvent was evaporated. DCM was added to the residue. The organic layer was washed with sat. NaHCO₃. The aqueous layer was backed extracted with DCM. The combined organic layers were dried over Na₂SO₄, filtered and evaporated. The residue w!as purified on silica gel column twice to give a slightly brown solid which was recrystalized from DCM to gixve cis-4 (4-dimethylamino-quinazolin-2-ylamino)-cyclohexanecarboxylic acid amide (20.6 g, 44%) as yellow crystals.

ESI-MS m/e 314 M+H⁺; ¹H NMR (400 MHz, DMSO-d₆) δ 8.19 (b, 1 H), 8.15 (d, J=8.4 Hz, 1 H), 7.77 (t, J=8 Hz, 1 H), 7.42 (d, J=7.2 Hz, 1H), 7.35 (t, J=8.4 Hz, 1 H), 7.21 (s, 1 H), 6.74 (s, 1 H), 4.12 (m, H), 3.46 (b, 6 H), 2.24 (m, 1 H), 1.79-1.61 (m, 8 H).

### Step E: Synthesis of cis-N²-(4-aminomethyl-cyclohexyl)-N⁴,N⁴-dimethyl-quinazoline-2,4-diamine

To a stirred solution of cis-4-(4-dimethylamino-quinazolin-2-ylamino)-cyclohexane carboxylic acid amide (18.78 g, 60 mmol) in 200 mL of THF was added a solution of 1M BH₃ in THF (300 mL, 300 mmol). The mixture was refluxed for 2 hr. After cooling the reaction mixture to 0° C., 100 mL of 4 M HCl and 200 mL of methanol were added. The solvents were removed under reduced pressure. The mixture was treated with 1M NaOH and the aqueous phase was extracted with dichloromethane. The organic layers were combined, dried ovir sodium sulfate, concentrated under reduced pressure, and purified on silica gel column to give cis-N⁷-(4-aminomethyl-cyclohexyl)-N′, N′-dimethyl-quinazoline-2,4-diamine as a white solid (10.6 g, 59%).

ESI-MS m/e 300 M+H⁺; ¹H NMR (400 MHz, DMSO-d₆) δ 7.84 (d, J=8.4 Hz, 1 H), 7.46 (t, J=6.8 Hz, 1 H), 7.26 (d, J=8.4 Hz, 1 H), 6.99 (t, J=6.8 Hz, 1 H), 6.28 (brs, 1 H), 4.02 (m, 1 H), 3.19 (brs, 6 H), 2.47 (d, J=6.8 Hz, 2 H), 2.73 (m, 2 H), 1.68-1.33 (m, 9 H).

### Step F: Synthesis of N-[(cis-4-{[4-(dimethylamino)quinazolin-2-yl]amino}cyclohexyl)-methyl]-N′-[2(trifluoromethoxy)phenyl]urea trifluoroacetate

A solution of cis-N²-(4-aminomethyl-cyclohexyl)-N⁴,N⁴-dimethyl-quinazoline-2,4-diamine (30 mg, 0.1 mmol) and 2-trifluoromethoxy phenylisocyanate (20 mg, 0.1 mmol) in 0.5 mL of DMSO was stirred at room temperature overnight. DMSO (0.5 mL) was added and the reaction mixture was purified by prep LCMS. The fractions contained the product were combined and lyophilized to give N-[(cis-4-{[4-(dimethylamino)quinazolin-2-yl]amino}cyclohexyl)methyl]-N′-[2-(rrifluoromethox-)phenyl]urea trifluoroacetate (21 mg, 3.4%) as a white solid.

ESI MS m/e. 503 M+H⁺; ¹H NMR (400 MHz, DMSO-d₆) δ 12.10 (brs, 1 H), 8.23 (d, J=8.0 Hz, 1, H), 8.15 (d, J=8.0 Hz 1 H), 8.14 (s, 1 H), 8.09 (brs, 1 H), 7.75 (m, 1 H), 7.43-7 24 m, 4 H), 6.98 (m, 2 H), 4.15 (m, 1H), 3.46 (brs, 6 H), 3.05 (m, 2 H), 1.77-1.35 (m, 9 H).

### Example 895

### 2-(4-Chlorophenoxy)-N-(cis-4-{[4-(dimethylamino)quinazolin-2-yl]amino}cyclohexyl)-nicotinamide trifluoroacetate

### Step A: Synthesis of cis-[4-(4-dimethlyamino-quinazolin-2-ylamino)-cyclohexyl]-carbamic acid tert-butyl ester

To a solution of cis-(4-amino-cyclohexyl)-carbamic acid tert-butyl ester (0.5 g, 0.0023 mol) in 1 mL 2-propanol was added (2-chloro-quinazolin-4-yl)-dimethyl-amine (0.53, 0.0026 mol) and DIEA (1.22 mL, 0.0070 mol). The mixture was heated in a microwave synthesizer at 170° C. for 1 hour. The reaction was repeated 39 more times (20 g total material) and the reaction mixtures were pooled. The solvent was evaporated and the material subjected to chromatography (2-4% 2M NH₃ in MeOH/CH₂Cl₂) to yield cis-[4-(4-dimethlylamino-quinazolin-2-ylamino)-cyclohexyl-carbamic acid tert-butly ester (22.1 g 0.057 mol, 61 %) as a colorless oil.

ESI MS m/e 386.4 M+H⁺; ¹H NMR (400 MHz, DMSO-d₆) δ 7.85 (d, J=8.0 Hz, 1 H), 7.47 (t, J=8.4 Hz, 1 H), 7.27 (d, J=8.0 Hz, 1 H), 7.00 (t, J=7.6 Hz, 1 H)j 6.60 (brs, 1 H), 6.18 (brs, 1 H), 3.9S-3.SS (m, 1 H), 3.39 (brs, 1 H), 3.19 (s, 6 H), 1.77-1.71 (m, 2 H), 1.68-1.52 (m, 6 H), 1.38 (s, 9 H).

### Step B: Synthesis of cis-N²(4-amino-cyclohexyl)-N⁴,N⁴-dimethyl-quinazolin-2,4-diamine

To a solution of cis-(4-(4-dimethylamino-quinazolin-2-ylamino)-cyclohexyl]-carbamic acid tert-butyl ester (22.1 g, 0.057 mol) in CH₂Cl₂ was added TFA (10 mL, 0.13 mol). The solution was stirred at room temperature for 4 hours. The excess solvent was evaporated off and the resulting oil was dissolved in CH₂Cl₂. The organic later was extracted with a dilute-NaOH (aq) solution. The aqueous layer was extracted twice more with CH₂Cl₂ and the organic layers combined, dried over MgSO₄, and concentrated. The resulting precipitate was crystallized in ether and hexanes to yield cis-N²-(4-amino-cyclohexyl)-N⁴,N⁴-dimethyl-quinazolin-2,4-diamine (15.0 g, 0.053 mol, 92%) as a pale yellow solid.

ESI MS m/e 286.2 M+H⁺; ¹H N-NMR (400 MHz, DMSO-d₆) δ 7.84 (d, J=8.4 Hz, 1 H), 7.45 (t, J=6.8 Hz, 1 H), 7.26 (d, J 8.4 Hz, 1 H), 6.99 (t, J=7.6 Hz, 1 H), 6.20 (brs, 1 H), 3.90-3.89 (m, 1 H), 3.18 (s, 6 H), 2.79 (s, 1 H), 1.74-1.71 (m, 2 H), 1.57-1.41 (m, 8 H).

### Step C: Synthesis of 2-(4-chlorophenoxy)-N-(cis-4-{[4-(dimethylamino)quinazolin-2-yl]amino}cyclohexyl)nicotinamide trifluoroacetate

To a solution of cis-N²-(4-amino-cyclohexyl-N⁴,N⁴-dimethyl-quinazolin-2,4-diamine (28.5 mg, 0.1 mmol) in 0.5 mL DMF was added 2-(4-chlorophenoxy)nicotinic acid (24.9 mg, 0.1 mmol), HATU (45.6 mg, 0.12 mmol), and DIEA (34.8 L, 0.2 mmol). The reaction mixture was stirred for a couple of hours, and the compound was then subjected to purification by prep LCMS to yield 2-(4-chlorophenoxy)-N-(cis-4-{[4-(dimethylamino)quinazolin-2-yl]amino}, cyclohexyl)nicotimide trifluoroacetate (15 mg, 0.029 mmol, 29%) as a white solid.

ESI-MS m/e 517.4 M+H⁺; ¹HMR (400 MHz, DMSO-d₆) δ 12.2 (s, 1 H), 8.58 (d, J=8.0 Hz 1 H), 8.48-8.39 (m, 2 H), 8.29 (d, J=8.0 Hz, 1 H), 8.13 (brs, 1 H), 8.02 (t, J=4.0 Hz, 1 H), 7.75 (m, 3 H), 7.61 (t, J=8.0 Hz, 1 H), 7.50 (m, 3 H), 4.25 (brs, 1 H), 4.21 (brs, 1 H), 3.69 (brs, 6 H), 2.00-1.80 (m, 8 H).

### Example 8961

### N-(cis-4-{[4-(Dimethylamino)quinazolin-2-yl]amino}cyclohexyl)-2-(4-fluorophenoxy)-nicotinamide trifluoroacetate

### Step A: Synthesis of cis-2-chloro-N-[4-(4-dimethylamino-quinazolin-2-ylamino)-cyclohexyl]-nicotinamide

To a solution of cis-N²-(4-amino-cyclohexyl)-N⁴,N⁴-dimethyl-quinazolin-2,4-diamine (1.0 g, 3.5 mmol) in 18 mL CH₂Cl₂ was added 2-chloronicotinyl chloride (616.7 mg, 3.5 mmol), DIEA (1.2 mL, 7.0 mmol). The reaction mixture was stirred for 30 minutes at room temperature, the solvent was removed under vacuum, and the residue was purified by column chromatography on silca gel (2-4% 2M NH₃ in CH₃OH/CH₂Cl₂) to yield cis-2-chloro-N-[4-(4-dimethylamino-quinazolin-2-ylamino)-cyclohexyl]-nicotinamide (0.71 g, 47%). ESI-MS m/e 425.2 M+H⁺; ¹ NMR (400 MHz, DMSO-d₆) δ 8.59 (brs, 1 H), 8.46 (d, J=4.0 Hz, 1 H), 8.30 (brs, 1 H) 8.18 (d, J=8.0 Hz, 1 H), 7.87 (d, J=8.0 Hz, 1 H), 7.79 (t J=8.0 Hz, 1 H), 7.53-7.43 (m, 2 H), 7.37 (t, J=8.0 Hz, 1 H), 4.09 (brs, 1 H), 3.93 (brs, 1 H), 3.57 (brs, 6 H), 1.90-1.62 (m, 8 H).

### Step B:. Synthesis of N-(cis-4-{[4-(dimethylamino)quinazolin-2-yl]amino}cyclohexyl)-2-(4-fluorophenoxy)nicotinamide trifluoroacetate

cis-2-Chloro-N-[4-(4-dimethlamino-quinazolin-2-ylamino)-cyclohexyl]-nicotinamide (30 mg, 0.07 mmol) was added into a stirred solution of 4-fluorophenol (7.93mgn. 0.07 mmol) and 60% NaH in mineral oil (5.6 mg, 0.14 mmol) in 0.5 mL DMA. The mixture was heated in a microwave synthesizer at 250° C. for 1 hour. The compound was then subjected to purification by prep LCMS to yield N-(cis-4-{[4-(dimethylamino)quinazolin-2-yl]amino}cyclohexyl)-2-(4-fluorophenoxy) nicotinamide trifluoroacetate (10.3 mg, 0.021 mmol 30%) as a white solid.

ESI-MS 501.3 M+H⁺; ¹H NMR (400 MHz, DMSO-d₆) δ 12.2 (s, 1 H), 8.51 (brs, 1 H), 8.38-8.34 (m, 2 H), 8.26 (d, J=8.0 Hz, 1 H), 8.17 (brs, 1 H), 7.98 (t. J=8.0 Hz, 1 H), 7.63 (brs, 1 H), 7.57 (t, J=8.0 Hz, 1 H), 7.47-7.40 (m, 5 H), 4.20 (brs, 1 H), 4.17 (brs, 1 H), 3.66 (brs, 6 H), 2.00-1.94 (m, 8 H).

### Example 897

### N-(cis-4-{[4-(Dimethylamino)quinazolin-2-yl]amino}cyclohexyl)-2-(4-methoxyphenoxy)-nicotinamide trifluoroacetate

### Step A: Synthesis of N-(cis-4-{[4-(dimethylamino)quinazolin-2-yl]amino}cyclohexyl)-2-(4-methoxyphenoxy)nicotinamide trifluoroacetate

Using a similar procedure as described in step B of Example 896, the title compound was obtained.

ESI-MS m/e 513.4 M+H⁺; ¹H NMR (400 MHz, DMSO-d₆) δ 11.8 (s, 1 H) 8.14 (brs, 1 H) 8.00(m, 2 H), 7.91 (brs, 1 H), 7.80 (brs, 1 H), 7.62 (t, J=8.0 Hz, 1 H), 7.27 (brs, 1 H), 7.21 (t, J=8.0 Hz, 1 H), 7.04 (q, J=4.0 Hz, 1 H), 6.99 (d, J=12.0 Hz, 2 H), 6.80 (d, J=12.0 Hz, 2 H), 3.82-3.76, (brs, 2 H), 3.40-3.30 (m, 6 H), 1.61-1.50 (m, 8 H).

### Example 898

### N-(cis-4-{[4-(Dimethylamino)quinazolin-2-yl]amino}cyclohexyl)-2-(3-methylphenoxy)-nicotinamide trifluoroacetate

### Step A: Synthesis of N-(cis-4-{[4-(dimethylamino)quinazolin-2-)yl]amino}cyclohexyl)-2-(3-methylphenoxy)nicotinamide trifluoroacetate

Using a similar procedure as described in step B of Example 896, the title compound was obtained.

ESI-MS m/e 497.4 M+H⁺; ¹H NMR (400 MHz, DMSO-d₆) δ 12.0 (brs, 1 H), 8.26 (d, J=4.8 Hz, 1 H), 8.18 (m, 2 H), 8.07 (d, J=6.8 Hz, 1 H), 7.88 (brs, 1 H), 7.77 ( t, J=8.0 Hz, 1 H), 7.43 (brs, 1 H), 7.36 (t, J=8.0 Hz, 1 H), 7.27 (t, J=8.0 Hz, 1 H), 7.20 (q, J=8.0 Hz, 1H), 7.02-6.96 (m, 3 H), 4.10-3.90 (m, 2 H), 3.80-3.20 (m, 6 H), 2.30 (s, 3 H), 1.78-1.50 (m, 8 H).

### Example 899

### N(cis-4-{[4-(Dimethylamino)quinazolin-2-yl]amino}cyclohexyl)-2-(2-methoxyphenoxy)-nicotinamide trifluoroacetate

### Step A: Synthesis of N(cir-4-{[4(dimethylamino)quinazolin-2-yl]amino}cyclohexyl)-2-(2-methoxyphenoxy)nicotinamide trifluoroacetate

Using a similar procedure as described in step B of Example 896, the title compounds was obtained.

ESI-MS m/e 513.2 M+H⁺; ¹H NMR (400 MHz, DMSO-d₆) δ 11.9 (s, 1 H), 8.15-8.12 (m, 4 H), 7.88 (brs, 1 H), 7.78 (t, J=8.0 Hz, 1 H), 7.42 (brs, 1 H), 7.30-7.10 (m, 4 H), 7.14 ( d, J=8.0 Hz, 1 H), 7.00 (t, J=8.0 Hz, 1 H), 4.15 (brs, 2 H), 3.69 (s, 3 H), 3.39 (brs, 6 H), 1.80-1.50 (m, 8 H).

### Example 900

### 2-(4-Bromophenoxy)-N-(cis-4-{[4-(dimethylamino)quinazolin-2-yl]amino}cyclohexyl)-nicotinamide trifluoroacetate

### Step A: Synthesis of 2-(4-bromophenoxy)-N-(cis-4-{[4-(dimethylamino)quinazolin-2-yl]amino}cyclohexyl)nicotinamide trifluoroacetate

Using a similar procedure as described in step B of Example 896, the title compounds was obtained.

ESI-MS m/e 563.2 M+H⁺; ¹H NMR (400 MHz, DPSO-d₆) δ 11.9 (s, 1 H), 8.16 (d, J==8.0 Hz, 1 H), 8.02-7.98 (m, 2 H), 7.88 (d, J=8.0 Hz, 1 H), 7.83 (brs, 1 H), 7.62 (t, J=8.0 Hz, 1 H), 7.42 (d, J=8.0 Hz, 2 H), 7.27 (brs, 1 H), 7.20 (t, J=8.0 Hz, 1 H), 7.03-7.05 (q, J=4.0 Hz, 1 H), 7.03 (d, J=12.0 Hz, 2 H), 3.83 (brs, 2 H), 3.29 (brs, 5 H), 1.59-1.50 (m, 8 H).

### Example 901

### N-(cis-4-{[4-(Dimethylamino)quinazolin-2-yl]amino}cyclohexyl)-2,6-dimethoxynicotinamide trifluoroacetate

### Step A: Synthesis of N-(cis-4-{[4-(dimethylamino)quinazolin-2-yl]amino}cyclohexyl)-2,6-dimethoxynicotinamide trifluoroacetate

To a solution of cis-N²-(4-amino-cyclohexyl)-N⁴,N⁴-dimethyl-quinazolin-2,4-diainine (28.5 mg, 0.1 mmol) in 0.5 mL DMF was added 2,6-dimethoynicotinic acid (18.3 mg, 0.1 mmol, HATU (45.6 mg, 0.12 mmol), and DIEA (34.8 L, 0.2 mmol). The reaction mixture was stirred for a couple of hours, and the compound was then subjected to purification by prep LCMS to yield N-(cis-4-{[4-(dimethylamino)quinazolin-2-yl]amino}cyclohexyl)-2,6-dimethoxynicotinamide tritluoroacetate (9.9 mg, 0.022 mmol, 22%) as a white solid.

ESI-MS m/e 451.2 M+H⁺; ¹H NMR (400 Nz, DMSO-d₆) δ 12.5 (s, 1 H), 8.42 (brs, 1 H), 8.13 (dd, J=4.0, 4.0 Hz, 2 H), 7.86 (brs, 1 H), 7.74 (t, J=8.0 Hz, 1 H), 7.39 (brs, 1 H), 7.32 (t, J=8.0 Hz, 1 H), 6.47 (d, J=8.0 Hz, 1 H), 4.02 (s, 3 H), 3.95 (brs, 1 H), 3.85 (s, 3 H), 3.68 (brs, 1 H), 3.42 (brs, 6 H), 1.80-1.68 (m, 8 H).

### Example 902

### N²-{(1S,3R)-3-[(3,5-Dichlorobenzyl)amino]cyclopentyl}-N⁴,N⁴-dimethylquinazoline-2,4-diamine bistrifluoroacetate

### Step A: Synthesis of (1S,3R)-cis-(3-tert-butoxycarbonylamino-cyclopentyl)-carbamic acid benzyl ester

(1R,3 S)-N-Boc-1-aminocyclopentane-3-carboxylic acid (5.00 g, 21.8 mmol), diphenylphosphoryl azide (4.69 mL, 21.8 mmol), and triethylamine (3.04 mL. 21.8 mmol) were combined in benzene (30 mL) at room temperarture. The mixture was heated to 80° C. and stirred 1 hr. Benzylalcohol (2.26 mL, 21.8 mmol) was added and the mixture was heated to 110° C. for 1 hr. The mixture was concentrated and ethyl acetate was added. The organic phase was washed with water, saturated aqueous NaHCO₂, and brine, dried over Na₂SO₄, filtered, and concentrated. The crude product was purified by flash chromatography (silica gel 20% ethyl acetate in hexanes) to give (1S,3R)-cis-(3-tert-butoxycarbonylamino-cyclopentyl)-carbamic acid benzyl ester (5.00 g, 69%) as a white solid.

ESI-MS m/e 335 M+H⁺; ¹H NMR (400 MHz, DMSO-d₆) δ 7.25 (m, 5 H), 6.83 (m, 2 H), 4.98 (s, 2 H), 3.77 (brs, 1 H), 2.13 (dt, J=12.8, 7.6 Hz, 1 H), 1.75 (d, J=7.2 Hz, 2 H), 1.43 (m, 2 H), 1.38), (s, 9 H), 1.22 (m, 2 H).

### Step B: Synthesis of (1R,3S)-cis-(3-amino-cyclopentyl)-carbamic acid tert-butyl ester

(1S,3R)-(3-tert-Butoxycarbonylamino-cyclopent)-carbamic acid benzyl ester (4.73 g, 14.2 mmol) and 10% Pd/C (0.24 g) were combined in methanol (27 mL) at room temperature. The mixture stirred for 4 days under a hydrogen gas atmosphere, was filtered through celite and concentrated to give (1R,3S)-cis-(3-amino-cyclopentyl)-carbamic acid tert-butyl ester as a yellow oil (2.84 g) (crude).

ESI-MS m/e 201 (M+H)⁺; ¹H NMR (400 MHz, DMSO-d₆) δ 6.8 (brs, 1 H), 3.70 (m, 1 H), 2.10 (brs, 2 H), 1.97 (dt, J=12.8, 6.8 Hz, 1 H), 1.70 (m, 2 H), 1.43 (m, 2 H), 1.38 (s, 9 H), 1.18 (m, 2 H).

### Step C: Synthesis of (1S,3R)-cis-N-(3-amino-cyclopent)-N⁴,N⁴-dimethyl-quinazoline-2,4-diamine

(2-Chloro-quinazolin-4-yl)-dimethyl-amine (0.100 g, 0.48 mmol), (1R,3S)-(3-amino-cyclopentyl)-carbamic acid tert-butl ester (0.096 g, 0.48 mmol), and diisopropylethylamine (0.126 mL. 0.72 mmol) were combined in isopropanol (1 mL) at room temperature. The mixture was heated to 160° C. for 40 min. utilizing a Smith synthesizer microwave apparatus. Trifluoroacetic acid (1 mL, neat) was added and the mixture was heated to 100° C. for 30 min. Then it was concentrated, neutralized with saturated aqueous NaHCO₃, concentrated, extracted with methanol, and concentrated again to give (1S,3R)-cis-N²-(3-amino-cyclopentyl)-N⁴,N⁴-dimethyl-quinazoline-2,4-diamine as a yellow gum (0.130 g) (crude).

ESI-MS m/e 272 M+H⁺; ¹H NMR (400 MHz, DMSC-d₆) δ 8.76 (brs, 1 H), 8.17 (d, J=7.6 Hz 1 H), 7.77 (d J=7.2 Hz, 1 H), 7.40 (brs, 1 H), 7.35 (d, J=7.6 Hz, 1 H), 3.80 (m, 1 H), 3.40 (s, 6 H), 2.20 (m, 1 H), 1.98 (brs, 2 H) 1.70 (m, 2 H), 1.43 (m, 2 H), 1.18 (m, 2 H).

### Step D: Synthesis of N²-{(1S,3R)-3-[(3,5-dichlorobenzylamino]cyclopentyl}-N⁴,N⁴-dimethyl quinazoline-2,4-diamine bistrifluoroacelate

(1S,3R)-N²-(3-Amino-cyclopentyl)-N⁴,N⁴-dimethly-quinazoline-2,4-diamine (0.065 g, 0.24 mmol) and 2,4-dimethoxybenzaldehyde (0.040 g, 0.24 mmol) were combined in methanol (1 mL) at room temperature. After stirring for 1 hr, sodium triacetoxyborohydride (0.204 g, 0.96 mmol) was added and the mixture as heated to 150° C. for 40 min. utilizing a Smith Synthesizer microwave apparatus. Water (1 mL) was added and the product was purified to give N²-{(1S,3R)-3-[(3,5-dichlorobenzyl)amino]cyclopentl}-N⁴,N⁴-dimethyl quinazoline-2,4-diamine bistrifluoroacetate as a white solid (0.070 g, 45%).

ESI-MS m/e 422 M+H⁺; ¹H NMR (400 MHz, DMSO-d₆) δ 9.32 (brs, 1 H), 8.17 (d, J=7.6 Hz, 1 H), 7.77 (t, J=7.2 Hz, 1 H), 7.69 (s, 1 H), 7.61 (s, 1 H), 7.60 (s, 1 H), 7.40 (brs, 1 H), 7.35 (t, J=7.6 Hz, 1 H), 4.33 (brs, 1 H), 3.58 (m, 2 H), 3.40 (s, 6 H), 2.20 (m, 1 H), 2.06 (brs, 1 H), 1.70 (m, 2 H), 1.43 (m, 2 H), 1.18 (m, 2 H).

### Example 903

### 6-(3-Chlorophenoxy)-N-(cis-4-{[4-(dimethylamino)quinazolin-2-yl]amino}cyclohexyl) nicotinamide trifluoroacetate

### Step A: Synthesis of cis-6-chloro-N-[4-(4-dimethylamino-quinazolin-2-ylamino)-cyclohexyl]-nicotinamide

To a solution of cis-N′-(4-amino-cyclohexyl)-N⁴,N⁴-dimethyl-quinazolin-2,4-diamine (1.8 g, 6.3-mmol) in 3 mL CH₂Cl₂ was added 6-chloronicotinyl choride (1.1 g, 6.3 mmol), DIEA (2.19 mL, 12.6 mmol). The reaction mixture was stirred for 30 minutes at room temperature, the solvent was removed under vacuum, and the residue was purified by column chromatography on silica gel (2-4% 2M, NH₃ in CH₃OH/CH₂Cl₂=5:10) to yield cis-6-chloro-N-[4-(4-dimethylamino-quinazolin-2-ylamino)-cyclohexyl]-nicotinamide (1.07 g, 40%).

ESI-MS m/e 425.0 M+H⁺; ¹H NMR (400 MHz, DMSO-d₆) δ 8.76 (brs, 1 H), 8.46 (brs, 1 H) 8.37 (brs, 1 H), 8.19 (dd, J=8.0, 4.0 Hz, 1 H), 8.12 (d, J=8.0 Hz, 1 H), 7.74 (t, J=8.0 Hz, 1 H), 7.59 (d, J=8.0 Hz, 1 H), 7.40 (brs, 1 H), 7.32 (t, J=8.0 Hz, 1 H), 3.99 (brs, 1 H), 3.86 (brs, 1 H), 3.30 (brs, 6 H), 1.85-1.62 (m, 8 H).

### Step B: Synthesis of 6-(3-cblorophenoxy)-N-(cis-4-{[4-(dimethylamino)q uinazolin-2-yl]amino}-cyclohexyl)nicotinamide trifluoroacetate

cis-6-Chloro-N-[4-(4-dimethylamino-quinazolin-2-ylamino)-cyclohexyl]-nicotinamide (30 mg, 0.07 mmol) was added into a stirred solution of 3-chlorophenol (17.9 mg, 0.14 mmol) and 60% NaH in mineral oil (5.6 mg, 0.14 mmol) in 0.5 mL DMA. The mixture was heated in a microwave synthesizer at 250° C. for 1 hour. The compound was then subjected to purification by prep LCMS to yield 6-(3-chlorophenoxy)-N-(cis-4-{[4-(dimethylamino)quinazolin-2-yl]amino}cyclohexyl)nicotinamide trifluoroacetate (8.2 mg, 0.016 mmol, 23 %) as a white solid.

ESI-MS m/e 517.02 M+H⁺; ¹H NMR(400 MHz, DMSO-d₆) δ 12.5 (s, 1 H), 8.63 (s, 1 H), 8.37 (brs, 1 H), 8.31 (dd, J=8.0, 4.0 Hz, 1 H), 8.21 (d, J=8.0 Hz, 1 H), 7.83 (t, J=8.0 Hz, 1 H), 7.56 (m, 2 H), 7.41 (m, 3 H), 7.22 (d, J=8.0 Hz, 2 H), 4.08 (brs; 1 H), 3.90 (brs, 1 H), 3.80-3.40 (brs, 6 H), 2.00-1.51 (m, 8 H).

### Example 904

### N-(cis-4-{[4-(Dimethylamino)quinazolin-2-yl]amino}cyclohexyl)-6-(3-fluorophenoxy)-nicotinamide trifluoroacetate

### Step A: Synthesis of N-(cis-4-{[4(dimethylamino)quinazolin-2-yl]amino}cyclohexyl)-6-(3-fluorophenoxy)nicotinamide trifluoroacetate

Using a similar procedure as described in step B of Example 903, the title compounds was obtained.

ESI-MS m/e 501.2 M+H⁺; ¹H NMR (400 MHz, DMSO-d₆) δ 1.20 (s, 1 H), 8.40 (brs, 1 H), 8.11 (brs, 1 H), 8.07-8.04 (m, 1 H), 7.97 (d, J=8.0 Hz, 1 H), 7.80 (brs, 1 H), 7.59 (t, J=8.0 Hz, 1 H), 7.29 (m, 2 H), 7.17 (t, J=8.0 Hz, 1 H), 6.97-6.86 (m, 3 H), 6.82 (d, J=8.0 Hz, 1 H), 3.85 (brs, 1 H), 3.77 (brs, 1 H), 3.40-3.20 (m, 6 H), 1.87-1.49 (m, 8 H).

### Example 905

### N-(cis-4-{[4-(Dimethylamino)quinazolin-2-yl]amino}cyclohexyl)-2-(3-fluorophenoxy)isonicotinamide trifluoroacetate

### Step A: Synthesis of cis-2-chloro-N-[4-(4-dimethylamino-quinazolin-2-ylamino)-cyclohexyl]-isonicotinamide

To a solution of cis-N²-(4-amino-cyclolexyl)-N⁴,N⁴-dimethyl-quinazolin-2,4-diamine (1.0 g, 3.5 mmol) in 18 mL CH₂Cl₂ was added 2-chlorophvridine-4-carbonyl chloride (616.7 mg, 3.5 mmol). The reaction mixture was stirred for 30 minutes at room temperature, the solvent was removed under vacuum, and the residue was purified by column chromatography on silica gel (2-4% 2M HN₃ in CH₃OH/CH₂Cl₂=5:0) to yield cis-2-chloro-N-[4-(4-dimethylamino-quinazolin-2-ylamino)-cyclohexyl]-isonicotinamide (0.79 g, 54%) as a white solid.

ESI-MS m/e 425.0 M+H⁺; ¹H NMR (400 MHz, DMSO-d₆) δ 8.58 (brs, 1 H), 8.50 (d, J=8.0 Hz, 1 H), 8.27 (brs, 1 H), 8.13 (d, J=8.0 Hz, 1 H), 7.81 (s, 1 H), 7.74-7.69 (m, 2 H), 7.40 (brs, 1 H), 7.32 (t, J=8.0 Hz, 1 H), 3.99 (brs, 1 H), 3.85 (brs, 1 H), 3.42 (brs, 6 H), 1.84-1.69 (m, 8 H).

### Step B: Synthesis of N-(cis-4-{[4-(dimethylamino)quinazolin-2-yl]amino}cyclohexyl)-2-(3-fluorophenoxy)isonicotinamide trifluoroacetate

cis-2-Chloro-N-[4-(4-dimethylamino-quinazolin-2-ylamino)-cyclohexyl]-isonicotinamide (30 mg, 0.07 mmol) was added into a stirred solution of 3-fluorophenol (6.34 μl, 0.07 mmol) and 60% NaH in mineral oil (5.6 mg, 0.14 mmol) in 0.5 mL DMA. The mixture was heated in a microwave synthesizer at 250° C. for 1 hour. The compound was then subjected to purification by prep LCMS to yield N-(cis-4-{[4-(dimethylamino)quinazolin-2-yl]amino}cyclohexyl)-2-(3-fluorophenoxy)isonicotinamide trifluoroacetate (7.3 mg, 0.0146 mmol, 21%) as a white solid.

ESI-MS m/e 501.4 M+H³⁰ ; ¹H NMR (400 MHz, DMSO-d₆) δ 12.1 (s, 1 H), 8.58 (brs, 1 H), 8.28 (d, J=4.0 Hz, 1 H), 8.18 (d, J=8.0 Hz, 1 H), 7.98 (brs, 1 H), 7.79 (t, J=8.0Hz, 1 H), 7.52 (d, J=4.0 Hz, 1 H), 7.43. (m, 3 H), 7.34 (t, J=8.0 Hz, 1 H), 7.10-7.06 (m, 2 H), 7.00 (d, J=4.0 Hz, 1 H), 4.07 (brs, 1 H), 3.97 (brs, 1 H), 3.50 (brs, 6 H),1.89-1.75 (m, 8 H).

### Example 906

### N-(cis-4-{[4-(Dimethylamino)quinazolin-2-yl]amino}cyclohexyl)-2-(4-fluorophenoxy)isonicotinamide trifluoroacetate

### Step A: Synthesis of N-(cis-4-{[4-(dimethylamino)quinazolin-2-yl]amino}cyclohexyl)-2-(4-fluorophenoxy)isonicotinamide trifluoroacetate

Using a similar procedure as described in step B of Example 905, the title compound was obtained.

ESI-MS m/e 501.3 M+H⁺; ¹H NMR (400 MHz, DMSO-d₆) δ 12.5 (s, 1H), 8.58 (brs, 1 H), 8.23 (brs, 1 H),8.22 (d, J=4.0 Hz, 1 H) 8.18(d, J=8.0 H), 7.8 (t, J=8.0 Hz, 1 H) 7.47-7.30 (m,4 H), 7.28-7.14 (m, 4 H) 4.10 (brs, 1 H), 3.95 (brs, H), 3.47 (brs, 6 H), 2.00-1.50(m, 8 H).

### Example 907

### 2-(2,3-Dichlorophenoxy)-N-(cis-4-{[4-(dimethylamino)quinazolin-2-yl]amino}-cyclohexyl)acetamide trifluoroacetate

### Step A: Synthesis of 2-(2,3-dichlorophenoxy)-N-(cis-4-{[4-(dimethylamino)quinazolin-2-yl]amino}cyclohexyl)acetamide trifluoroacetate.

To a solution of cis-N²-(4-amino-cyclohexyl)-N⁴,N⁴-dimethyl-quinazolin-2,4-diamine (28.5 mg, 0.1 mmol) in 0.5 mL DMF was added 2,3-dichlorophenoxyacetic acid (18.2 mg, 0.1 mmol), HATU (45.6 mg, 0.12 mmol), and DIEA (34.8 μL, 0.2 mmol). The reaction mixture was stirred for a couple of hours, and the compound was then subjected to purification by prep LCMS to yield 2-(2,3-dichlorophenoxy)-N-(cis-4-{[4-(dimethlamino)quinazolin-2-yl]amino}cyclohexyl)acetamide trifluoroacetate (12. 3 mg, 27%) as a white solid.

ESI-MS m/e 488.2 M+H⁺; ¹H NMR (400 MHz, DMSO-d₆) δ 12.3 (s, 1 H), 8.16 (brs, 1 H), 8.12 (d, J=8.0 Hz 1 H), 7.81 (brs, 1 H), 7.74 (t, J=8.0 Hz, 1 H), 7.40 (brs, 1 H), 7.32 (t, J=8.0 Hz, 1 H), 7.27 (t, J=8.0 Hz, 1 H), 7.18 (d, J=8.0 Hz, 1 H), 6.99 (d, J=8.0 Hz, 1 H), 4.65 (s, 2 H), 3.95 (brs, 1 H). 3.76 (brs, 1 H), 3.41 (brs, 6 H), 1.72-1.62 (m 8 H).

### Example 908

### N-(cis-4-{[4-(Dimethylamino)quinazolin-2-yl]amino}cyclohexyl-2-(2-naphthyloxy)acetamide trifluoroacetate

### Step A: Synthesis of N-(cis-4-{[4-(dimethylamino)quinazolin-2-yl]amino}cyclohexyl)-2-(2-naphthyloxy)acetamide trifluoroacetate

To a solution of cis-N²-(4-amino-cyclohexy)-N⁴,N⁴-dimethyl-quinazolin-2,4-diamine (28.5 mg, 0.1 mmol) in 0.5 mL DMF was added 2-naphthoxyacetic acid (20 mg, 0.1 mmol), HATU (45.6 mg, 0.12 mmol), and DIEA (34.8 μL, 0.2 mmol). The reaction mixture was stirred for a couple of hours, and the compound was then subjected to purification by prep LCMS to N-(cis-4-{[4-(dimethylamino)quinazolin-2-yl]amino}cyclohexyl)-2-(2-naphthyloxy)acetamide trifluoroacetate (10.0 mg, 0.021 mmol, 21%) as a white solid.

ESI-MS m/e 470.4 M+H⁺; ¹H-NMR (400 MHz, DMSO-d₆) δ 12.1 (s, 1 H), 8.13 (d, J=12.0 Hz, 1 H), 8.02 (brs, 1 H), 7.93 (brs, 1 H), 7.80 (t, J=8.0 Hz, 2 H), 7.74-7.70 (m, 7 H), 7.41 (t, J=8.0 Hz, 2 H), 7.33 (m, ¹H), 7.20-7.17 (m, 1 H), 4.57 (s, 2H), 4.05 (brs, 1H), 3.76 (brs, 1H), 3.41 (brs, 6H), 1.71-1.62 (m, 8H).

### Example 909

### 2-(3,4-Difluorophenoxy)-N-(cis-4-{[4-(dimethylamino)quinazolin-2-yl]amino}cyclohexyl)-acetamide trifluoroacetate

### Step A: Synthesis of cis-2-bromo-N-[4-(4-dimethylamino-quinazolin-2-ylamino)-cyclohexyl]-acetamide

To a solution of cis-N²-(4-amino-cyclohexyl)-N⁴,N⁴-dimethyl-quinazolin-2,4-diamine (1.0 g, 3.5 mmol) in 18 mL CH₂Cl₂ was added bromoacetyl bromide (305 μL, 3.5 mmol) at 0° C. The reaction mixture was stirred for 2 hours, the solvent was removed under vacuum, and the residue was purified by column chromatography on silica gel (2-4% 2M NH₃ in CH₂OH/CH₂Cl₂ to yield cis-2-bromo-N-[4-(4-dimethylamino-quinazolin-2-ylamino)-cyclohexyl]-acetamide (0.95 g, 2.35 mmol, 67%), as a yellowish solid

ESI-MS m/e 406.2 M+H⁺; ¹H NMR (400 MHz, DMSO-d₆) δ 8.63 (brs, 1 H), 8.43 (brs, 1 H), 8.35 (d, J=8.0 Hz 1 H), 7.97 (t, J=8.0 Hz,1 H), 7.62 (brs, 1 H), 7.55 (t J=8.0 Hz, 1 H), 4.23 (brs, 1 H), 4.05 (s, 2 H), 3.89 (brs, 1 H), 3.70-3.60 (brs, 6 H), 2.00-1.75 (m, 8 H).

### Step B: Synthesis of 2-(3,4-difluorophenoxy)-N-(cis-4-{[4-(dimethylamino)quinazolin-2-6l]-amino}cyclohexyl)acetamide trifluoroacetate

cis-2-Bromo-N-[4-(4-dimethylamino-quinazolin-2-ylamino)-cyclohexyl]acetamide (60 mg, 0.15 mmol) was added into a stirred solution of 3,4-difluorophenol (19.3 mg, 0.15 mmol) and 60% NaH in mineral oil (11.8 mg, 0.30 mmol) in 1 mL DMA. The mixture was heated in a microwvave synthesizer at 250° C. for 1 hour. The compound was then subjected to purification by prep LCMS to yield 2-(3,4-difluorophenoxy)-N-(cis-4-{[4-(dimethylamino) quinazolin-2-yl]amino}-cyclohexyl)acetamide trifluoroacetate (32 mg, 0.07 mmol, 47%) as a white solid. ESI-MS m/e 456.2 M+H⁺; ¹H NMR (400 MHz, DMSO-d₆) δ 12.4 (s, 1 H), 8.25 (brs, 1 H), 8.2 (d, J=8.0 Hz, 1 H), 7.99 (brs, 1 H), 7.83 (t, J=S.0 Hz, 1 H), 7.49 (brs, 1 H), 7.43-7.36 (m, 2 H), 7.13-7.08 (m, 1 H), 6.82 (brs, 1 H), 4.55 (s, 2 H), 4.06 (brs, 1 H), 3.81 (brs, 1 H), 3.5 (brs, 6 H), 1.89-1.75 (m, 8 H).

### Example 910

### 2-(3,4-Difluorophenoxy)-N-(cis-4-{[4-(dimethylamino)quinazolin-2-yl]amino}cyclohexyl)-propanamide trifluoroacetate

### Step A: Synthesis of cis-2-bromo-N-[4-(4-dimethylamino-quinazolin-2-ylamine)-cyclohexyl]-propionamide

To a solution of cis-N-(4-anino-cyclohexyl)-N⁴,N⁴-dimethyl-quinazolin-2,4-diamine (1.0 g, 3.5 nmmol) in 18 mL CH₂Cl₂ was added 2-bromopropionyl bromide (189 μL, 1.75 mmol) at 0° C. The reaction mixture was stirred for 2 hours, the solvent was removed under vacuum, and the residue was purified by column chromatography on silica gel (2-4% 2M NH₃ in CH₃OH/CH₂Cl₂) to yield cis-2-bromo-N-[4-(4-dimethylamino-quinazolin-2-ylamino)-cyclohexyl]-propionamide (0.66 g, 45%) as a white solid.

ESI-MS m/e 410.2 M+H⁺; ¹H NMR (400 MHz, DMSO-d₆) δ 8.17 (m, 3 H), 7.76 (t, J=8.0 Hz, 1 H), 7.40 (brs, 1 H), 7.32 (t, J=8.0 Hz, 1 H), 7.55 (q, J=4.0 Hz, 1 H), 3.99 (brs, 1 H), 3.57 (brs, 1 H), 3.41 (brs, 6 H), 1.69-1.50 (m, 11 H).

### Step B: Synthesis of 2-(3,4-difluorophenoxy)-N-(cis-4-{[4-(dimethylamino)quinazolin-2-yl]-amino}cyclohexyl)propanamide trifluoroacetate

cis-2-Bromo-N-[4-(4-dimethylamino-quinazolin-2-ylamino)-cyclohexyl]-propionamide (60 mg, 0.14 mmol) was added into a stirred solution of 3,4-difluorophenol (18.6 mg, 0.14 mmol) and 60% NaH in mineral oil ( 1.4 mg, 0.29 mmol) in 1 mL DMA. The mixture was heated in a microwave synthesizer at 250° C. for 1 hour. The compound was then subjected to purification by prep LCMS to yield 2-(3,4-difluorophenoxy)-N-(cis-4-{8 4-(dimethyl]amino)quinazolin-2-yl]amino}cyclohexyl)propanamide trifluoroacetate (6.7 mg, 0.01 4 mmol, 10%) as a white solid.

ESI-MS m/e 470.4 M+H⁺; ¹H NMR (400 MHz, DMSO-d₆) δ 12.2 (s, 1 H), 8.19 (d, J=8.0 Hz, 1 H), 7.99 (brs, 1 H), 7.81 (t, J=8.0 Hz, 1 H), 7.46 (brs, 1 H), 7.39-7.31 (m, 2 H), 7.05-6.97 (m, 1 H), 6.75 (brs: 1 H), 4.80-4.73 (m, 1 H), 4.01(brs, 1 H), 3.71 (brs, 1 H), 3.47 (brs, 6 H), 1.62-1.47 (m, 8 H), 1.43 (d, J=4.0 Hz, 3 H).

### Example 911

### 2-(3,4-Difluorophenoxy)-N-(cis-4-{[4-(dimethylamino)quinazolin-2-yl]amino}cyclohexyl)-butanamide trifluoroacetate

### Step A: Synthesis of cis -2-bromo-N-[4-(4-dimethylamino-quinazolin-2-ylamino)-cyclohexyl]-butyramide

To a solution of cis-N²-(4-amino-cyclohexyl)-N⁴,N⁴-dimethyl-quinazolin-2,4-diamine (1.0 g, 3.5 mmol in 18 mL CH₂Cl₂ was added 2-bromobutyryl bromide (213 μL, 1.75 mmol) at 0° C. The reaction mixtuire was stirred for 2 hours, the solvent was removed under v acuum, and the residue was purified by column chromatography on silica gel (2-4% 2M NH₃ in CH₃OH/CH₂Cl₂) to yield cis-2-bromo-N-[4-(4-dimethylamino-quinazolin-2-ylamino-cyclohexyl]-butyramide (0.53 g,35 %) as a white solid.

ESI-MS m/e 434.2 M+H⁺; ¹H NMR (400 MHz, DMSO-d₆) δ 8.15 (brs,, 1 H), 8.12 (d, J=8.0 Hz, 2 H), 7.74 (t, J=8.0 Hz, 1 H), 7.40 (brs, 1 H), 7.32 (t, J=8.0 Hz, 1 H), 4.3.3 (t, J=8.0 Hz, 1 H), 3.93. (brs, 1 H). 3.66 (brs, 1 H), 3.41 (brs, 6 H), 2.01-1.87 (m, 1 H), 1.85-1.76 (m, 1 H), 1.70-1.59 (m, 8 H), 0.84 (t, J=8.0 Hz, 3 H).

### Step B: Synthesis of 2-(3,4-difluorophenoxy)-N-(cis-4-{[4-(dimethylamino)quinazolin-2-yl]amino}cyclohexyl)butanamide trifluoroacetate

cis-2-Bromo-N-[4-(4-dimethylamino-quinazolin-2-ylamino)-cyclohexyl]-butyramide (60 mg, 0.14 mmol) was added into a stirred solution of 3,4-difluorophenol (18.6 mg, 0.14 mmol) and 60% NaH in mineral oil (10.8 mg, 0.27 mmol) in 1 mL DMA. The mixture was heated in a microwave synthesizer at 250° C. for 1 hour. The compound was then subjected to purification by prep LCMS to yield 2-(3,4-difluorophenoxy)-N-(cis-4-{[4-(dimethylamino)quinazolin-2-yl]amino}cyclohexyl)butanamide trifluoroacetate (6.3 mg, 93%) as a white solid.

ESI-MS m/e 484.2 M+H⁺; ¹H NMR (400 MHz DMSO-d₆) δ 12.2 (s, 1 H), 8.12 (d, J=8.0 Hz, 2 H), 8.09 (brs, 1 H), 7.93 (brs, 1 H), 7.74 (t, J=8.0 Hz, 1 H), 7.40 (brs, 1 H), 7.32-7.24 (m, 2 H), 6.97-6.91 (m, 1 H), 6.70-6.67 (m, 1 H), 4.56 (t, J=4.0 Hz, 1 H), 3.95 (brs, 1 H), 3.67 (brs, 2 H), 3.41 (brs, 6 H), 1.84-1.77 (m, 2 H), 1.75-1.56 (m: 8 H), 0.90-0.81 (t, J=16.0 Hz 3 H).

### Example 912

### N²-(3-Chlorophenyl)-N-(cis-4-{[4-(dimethylamino)quinazolin-2-6l]amino}cyclohexyl)glycinamide bistrifluoroacetate

### Step A: Synthesis of N²-(3-chlorophenyl)-N-(cis-4-{[4-(dimethlamino)quinazolin-2-yl]-amino}cyclohexyl)glycinamide bistrifluoroacetate

To a solution of cis-2-bromo-N-[4-(4-dimethylamino-quinazolin-2-ylamino)-cyclohexyl]-acetamide (40 mg, 0.1 mmol) in 0.5 mL DFF was added 3-chloroaniline (11.6 μL, 0.11 mmol). The reaction mixture was stirred at 100° C., and another 0.5 mL of DMSO was added. The compound was then subjected to purification by prep LCMS to yield N²-(3-chlorophenyl)-N-(cis-4-{[4-(dimethylamino)quinazolin-2-yl]amino}cyclohexyl)glycinamide bistrifluoroacetate (6.0 mg, 8.3 %) as a white solid.

ESI-MlS m/e 453.2 M+H⁺; ¹H NMR (400 MHz, CD₃OD) δ 8.18 (d, J=8.4 Hz, 1 H), 7.77 (t, J=8.0 Hz, 1 H) 7.41 (m, 2 H), 7.11 (t, J=8.0 Hz, 1H), 6.66-6.51 (m, 3 H), 4.20 (brs, 1 H), 3.93 (brs, 1 H), 3.76 (s, 2 H), 3.54 (brs, 6 H), 1.87-1.17 (m, 8 H).

### Example 913

### 2-(3,5-Difluorophenyl)-N-[(cis-4-{[4-(dimethylamino)quinazolin-2-yl]amino}cyclohexyl)-methyl]-2-hydroxyacetamide trifluoroacetate

### Step A: Synthesis of cis-(4-amino-cyclohexylmethyl)-carbamic acid benzyl ester

To a solution of (4-aminomethyl-cyclohexl!)-carbamic acid tert-butyl ester (21.9 g, 96.0 mmol) in 400 mL CH₂-Cl₂ was added DIEA (16.6 mL, 96 mmol), CbzCl (11.4 mL, 79.7 mmol). The reaction mixture was stirred at room temperature for 3 hours, and the solvent was then removed under vacuum, and the residue was purified by column chromatography on silica gel (Hexane/EtOAc=1:1). The purified compound in 100 mL CH₂Cl₂ was added TFA (60 mL). The solution was stirred at room temperature for 2 hours. The excess solvent was evaporated off and the resulting oil was dissolved in CH₂Cl₂. The organic layer was extracted with a dilute NaCH (aq) solution. The aqueous layer was extracted twice more with CH₂Cl₂ and the organic layers combined, dried over MgSO₄, and concentrated to yield cis(4-amino-cyclohexylmethyl)-carbamic acid benzyl ester (20 g, 79%) as a yellow solid.

ESI-MS m/e 263.2 M+H⁺; ¹H NMR (400 MHz, DMSO-d₆) δ 7.82 (brs, 2 H), 7.39-7.29 (m, 6 H), 5.06 (s, 2 H), 3.15 (brs, 1 H), 2.98 (m, 1 H), ?,51(m, 1 H), 1.60-1.2-4 (m, 8 H).

### Step B: Synthesis of cis-[4-(4-dimethylamino-quinazolin-2-ylamino)-cyclohexylmethyl]-carbamic acid benzyl ester

To a solution of cis-[4-(4-dimethylamino-quinazolin-2-ylamino)-cyclohexylmethly]-carbamic acid benzyl ester (0.5 g, 1.9 mmol) in 1 mL 2-propanol was added (2-chloro-quinazolin-4-yl)-dimethyl-amine (0.33 g, 1.58 mmol) and DIEA (661 μL, 3.8 mmol). The mixture was heated in a microwave synthesizer at 150° C. for 1 hour. The reaction was repeated 39 more times (20 g total material) and the reaction mixtures were pooled. The solvent was evaporated and the material subjected to chromatography (2-4% 2M NH₃ in MeOH/CH₂Cl₂) to yield cis-[4-(4-dimethylamino-quinazolin-2-ylamino)-cyclohexylmethyl]-carbamic acid benzyl ester (16 g, 49%) as a yellowish oil.

ESI-MS m/e 434.2 M+H⁺; ¹H NMR (400 MHz, DMSO-d₆) δ 8.59 (brs, 1 H), 8.14 (d, J=8.0 Hz, 1 H), 7.76 (t, J=8.0 Hz, 1 H), 7.34 (d J=8.0 Hz, 1 H), 7.35 (m, 7 H), 5.06 (s, 2 H), 4.24 (brs, 1 H), 3.59 (brs, 6 H), 2.85 (brs, 2 H), 1.66-1.35 (m, 9 H).

### Step C: Synthesis of cis-N²-(4-aminomethyl-cyclohexyl)-N⁴,N⁴-dimethyl-quinazoline-2,4-diamine

To cis-[4-(4-dimetylamino-quinazolin-2-ylamino)-cyclohexylmethyl]-carbamic acid benzyl ester (16.0 g 37 mmol) in ethanol was added 10% Pd/C (1.6 g). The reaction mixture was stirred at room temperature under an H₂ atmosphere for 3 hours. The H₂ atmosphere was removed and the solution filtered though celite and concentrated to yield cis-N²-(4-aminomethyl-cyclohexyl)-N⁴,N⁴-dimethyl-quinazoline-2,4-diamine (11.2 g, 99%) as a yellowish solid.

ESI-MS m/e 300.2 M+H⁺; ¹H NMR (400 MHz, DMSO-d₆) δ 8.50 (brs, 1 H), 8/10 (d, J=127.0 Hz, 1 H), 7.71-7.61 (m, 3 H), 7.34 (d, J=8.0 Hz, 1 H), 7.27 (t, J=8.0 Hz, 1 H), 4.11 (brs, 1 H), 3.30 (brs, 6 H), 2.65 (brs, 2 H), 1.67-1.19 (m, 9 H).

### Step D: Synthesis of 2-(3,5-difluorophenyl)-N-[(cis-4-{[4-dimethylamino)quinazolin-2-yl]amino}cyclohexyl)methyl]-2-hydroxyacetamide trifluoroacetate

To a solution of cis-N²(4-aminonethyl-cyclohexyl)-N⁴,N⁴-dimethyl-quinazoline-2,4-diamine (29.9 mg, 0.1 mmol) in 0.5 mL DMF was added 3,5-difluoromandelic acid (18.8 mg, 0.1 mmol), HATU (45.6 mg, 0.12 mmol), and DIEA (34.8 μL, 0.2 mmol). The reaction mixture was stirred for a couple of hours, and the compound was then subjected to purification by prep LCMS to 2-(3,5-difluorophenyl)-N-[(cis-4-{[4-(dimethylamino)quinazolin-2-yl]amino}cyclohexyl) methyl]-2-hydroxyacetamide trifluoroacetate (29.5 mg, 51%) as a white solid. ESI-MS m/e 470.4 M+H⁺; ¹H NMR (400 MHz, CD₃OD) δ 8.16 (d, J=8.0 Hz, 1 H), 7.76 (t, J=8.4 Hz, 1 H), 7.39 (m, 2 H), 7.12 (m, 2 H), 6.86 (m, 1 H), 5.04 (s, 1 H), 4.21 (brs, 1 H), 3.53 (brs, 6 H), 3.21 (m, 2 H), 1.86-1.39 (m, 9 H).

### Example 914

### 2-(3,5-Difluorophenyl)-N-(cis-4-{[4-(dimethylamino)quinazolin-2-yl]amino}cyclohexyl)-2-hydroxyacetamide trifluoroacetate

### Step A: Synthesis of 2-(3,5-difluorophenyl)-N-(cis-4-{[4-(dimethylamino)quinazolin-2-yl]amino}cyclohexyl-2-hydroxyacetamide trifluoroacetate

To a solution of cis-N²-(4-amino-cyclohexyl)-N⁴,N⁴-dimethyl-quinazolin-2,4-diamine (28.5 mg, 0.1 mmol) in 0.5 mL DMF was added 3,5-difluoromandelic acid (18.8 mg, 0.1 mmol), HATU (45.6 mg, 0.12 mmol), and DIEA (34.8 μL, 0.2 mmol). The reaction mixture was stirred for a couple of hours, and the compound was then subjected to purification by prep LCMS to yield 2-(3,5-difluorophenyl)-N-(cis-4-{[4-(dimethylamino)quiazolin-2yl]amino}cyclolexyl)-2-hydroxy acetamide trifluoroacetate (20.5 mg, 0.045 mmol, 45%) as a white solid.

ESI-MS m/e 456.2 M+H¹; ¹H NMR (400 MHz, DMSO-d₆) δ 12.3 (s, 1 H), 8.29 (d, J=8.0Hz, 1 H), 8.18 (brs, 1 H), 7.91 (m, 2 )H, 7.58 (brs, 1 H), 7.49 (t, J=8.0 Hz, 1 H), 7.25-7.22 (m, 3 H), 6.55 (brs, 1 H), 5.13 (s, 1 H), 4.15 (brs, 1 H), 3.82 (brs, 1 H), 3.58 (brs, 6 H), 1.85-1.73 (m 8 H).

### Example 915

### cis-N-Benzyl-4-[(4-isopropylquinazolin-2-yl)amino]clcylhexanecarboxamide trifluoroacetate

### Step A: Synthesis of 2-chloro-4-isopropylquinazoline

2,4-Dichloroquinazoline (0.5 g, 2.5 mmol) and 1,2-bis(diphenylphosphino) ethane nickel (II) chloride (15 mg) were mixed with THF (10 mL), and the reaction was kept under an inert atmosphere. The reaction flask was cooled in a cold bath (˜20° C.), and isopropyl magnesium chloride (1.25 mL of 2M solution, 2.5 mmol) introduced into the reaction through a syringe. The reaction was slowly allowed to room temperature, and stirred ovennight. The reaction was quenched with addition of 1N-HCl (˜5 mL), diluted with water, and extracted with DCM (3×10 mL). Te organic layer was washed with aqueous NaHCO₃ (1×10 nmL) and wiater (1×10 mL), dried with MgSO₄, and concentrated. The crude was purified by column chromatography, (silica gel, hexanes: DCM=90:10 to 70:30) to give 0.11 g (20%) of 2-chloro-4-isopropylquinazoline as a white solid.

ESI MS m/e 207 M+H⁺; ¹H NMR (400 MHz, CDCl₃) δ 8.16 (d, J=8.0 Hz, 1 H), 7.97 (d, J=8.0 Hz, 1 H), 7.89 (t, J=8.0 Hz, 1 H), 7.63 (t, J=S.0 Hz, 1 H), 3.90 (m, 1 H), 1.44 (d, J=7.0 Hz, 6 H).

### Step B: Synthesis of cis-4-amino-cyclohexanecarboxylic acid ethyl ester hydrochloride

To a suspension of cis-aminocyclohexane-4-carboxylic acid (1.5 g, 10 mmol) in EtOH (15 mL) was added concentrated HCl (1.5 mL). The reaction was stirred for 2 h at 72° C. Removal of the volatile solvent under a vacuum gave cis-4-amino-cyclohexanecarboxylic acid ethyl ester hydrochloride (1.7 g, 96) as a white power, which was used directly to the next reaction without further purification.

ESI MS m/e 172 M+H⁺; ¹H NMR (400 MHz, DMSO-d₆) δ 4.43 (brs, 2 H), 4.05 (q, J=7.2 Hz, 2 H), 3.02 (brs, 1 H), 2.48 (m, 1 H), 1.93 (m, 2 H), 1.76 (m, 2 H) 1.43-1.57 (m, 4 H), 1.17 (t, J=7.2 Hz, 3 H).

### Step C: Synthesis of cis-4-(4-isopropyl-quinazolin-2-ylamino)-cyclohexane carboxylic acid ethyl ester

A solution of 2-chloro-4-isopropylquinazoline (0.26 g, 1.26 mmol) and cis-(4-ethoxycarbonyl)aminocyclohexane hydrochloride (0.26 g, 1 eq.) in IPA (2 mL) and DIEA (0.4 mL, 2 eq.) was reacted for 4 h at 160° C. in a Smith synthesizer. The reaction was purified from column chromatography (silica gel, DCM/MeOH=100:0 to 90:10) to give 0.25 g (58%) of cis-4-(4-isopropyl-quinazolin-2-yalamino)-cyclohexanecarboxylic acid ethyl ester.

ESI MS m/e 342 M+H⁺; ¹H NMR (400 MHz, CDCl₃) δ 7.90 (d, J=8.0 Hz, 1 H), 7.60 (m, 1 H), 7.55 (d, J=8.0 Hz, 1 H), 7.17 (t, J=8.0Hz, 1 H), 5.22 (d, J=7.0 Hz, 1 H), 4.21(brs, 1 H), 4.16 (q, J=7.0 Hz, 2 H), 3.74 (m, 1 H), 2.50 (m, 1 H) 1.96 (m, 2 H), 1.86-1.77 (m, 6 H), 1.36 (d, J=7.0 Hz, 6 H), 1.27 (t, J=7.0 Hz, 3 H).

### Step B: Synthesis of cis-4-(4-isopropl-quinazolin-2-ylamino)-cyclohexane carboxylic acid

A suspension of cis-4-(4-isopropyl-quinazolin-2-ylamino)-cyclohexane carboxylic acid ethyl ester (0.25 g, 0.7 mmol) in 4N-HCl (8 mL) was stirred for 3 h at 85° C. During the reaction, the heterovenous solution turned to be a clear solution, and then the precipitate was formed. The solid was filtered, washed with cold water several times, and dried to give 0.13 g, (58%) of cis-4)4-isopropyl-quinazolin-2-ylamino)-cyclohexane caboxylic acid as a white solid. ESI MS m/e 314 M+H⁺; ¹H NMR (400 MHz, DMSO-d₆) δ 12.25 (brs, 1 H), 9.56 (brs, 1 H), 8.40-8.26 (m, 2 H), 8.01 (m, 1 H), 7.59 (m, 1H), 4.31 (brs, 1 H), 4.03 (m, 1 H), 2.62 (brs, 1 H), 2.14 (m, 2 H), 1.93-1.66 (m, 6 H), 1.37 (d, J=6.4 Hz, 6 H).

### Step E: Synthesis of cis-N-benzyl-4-[(4-isopropylquinazolin-2-yl)amino]cyclohexanecarboxamide trifluoroacetate

cis-4-(4-Isoprop)l-quinazolin-2-ylamino)-cyclohexane carboxylic acid (20 mg, 0.06 mmol) and benzyl amine (7 mg, 0.06 mmol) was reacted in the presence of HATU (25 mg, 0.066 mmol and Et₃N (4 drops) at room temnperature for 16 hr. cis-N-benzyl-4-[(4-isopropylquiazolin-2-yl) amino]cyclohexanecarboxamide trifluoroacetate (13 mg, 40%) was obtained from a prep-HPLC.

ESI MS m/e 403 M+H⁺; ¹H NMR (400 MHz, DMSO-d₆) δ 9.06 (brs, 1 H), 8.24 (m, 2 H), 7.88 (brs, 1 H), 7.75-7.59 (m, 1 H), 7.45 (brs, 1 H), 7.17 (m, 2 H), 7.17 (m, 3 H), 4.24 (brs, 1 H), 4.23 (d, J=6.0 Hz, 2 H), 3.92 (m, 1 H), 2.33 (brs, 1 H), 1.95-1.58 (m, 8 H), 1.26 (d. J=6.4 Hz, 6 H).

### Example 916

### cis-N-(3-Chlorobenzyl)-4-[(4-isopropylquinazolin-2-yl)amino]cyclohexanecarboxamide trifluoroacetate

### Step A: Synthesis of cis-N-(3-chlorobenzyl)-4-[(4-isopropylquinazolin-2-yl)amino]-cyclohexancarboxamide triluoroacetate

Using a similar procedure as described in step E of Example 915, the title compound was obtained.

ESI MS m/e 437 M+H⁺; ¹H NMR (400 MHz, DMSO-d₆) δ 9.04 (brs, 1 H), 8.30 (t, J=5.4 Hz, 1 H), 8.20 (brs, 1 H), 7.86 (brs, 1 H), 7.71-7.57 (m, 1 H), 7.45 (brs, 1 H), 7.30-7.22 (m, 3 H), 7.15 (d, J=8.0 Hz, 1 H), 4.24 (brs, 1 H), 4.23 (d, J=6.0 Hz, 2 H), 3.92 (m, 1 H) 2.33 (brs, 1 H), 1.95-1.58 (m, 8H), 1.26 (d, J=6.6 Hz, 6 H).

### Example 917

### 3,4-Dichloro-N-[((1R,3S)-3-({[4-(dimethylamino)quinazolin-2yl]amino}cyclopentyl)methyl]-bemzamide trifluoroacetate

### Step A: Synthesis of cis-(1R,3S)-3-tert-butoxycarbonylamino-cyclopentanecarboxylic acid ethylformate ester

(1S,3R)-N-Boc-1-aminocyclopentane-3-carboxylic acid (10.00 g, 43.6 mmol) was dissolved in dichloromethane (100 mL) and cooled to −65° C. Triethylamine (9.19 mL, 65.9 mmol) and a solution of ethyl chloroformate (4.24 mL, 44.4 mmol) in dichloromethane (14 mL) were added and the mixture stirred at 0° C. for 1 hr. The mixture was acidified to pH ˜6 with 1N HCl (aq) and extracted with dichloromethane. The organic phase was washed with saturated aqueous NaHCO₃, water, and brine, dried over Na₂SO₄, filtered, and concentrated to give cis-(1R,3S)-3-tert-butoxycarbonnylamino-cyclopentanecarboxylic acid ethylformate ester as a clear oil.

ESI MS m/e 302, M+H⁺; ¹H N MNR (400 MHz, DMSO-d₆) δ 6.92 (brs, 1 H), 4.25 (q, J=7.2 Hz, 2 H), 3.78 (m, 1 H), 2.98 (m, 1 H), 2.16 (m, 2 H), 1.84 (m, 2 H), 1.80 (m, 2 H), 1.38 (s, 9 H), 1.25 (t, J=7.2 Hz, 3 H).

### Step B: Synthesis of cis-(1S,3R)-(3-hydroxymethyl-cyclopentyl)-carbamic acid tert-butyl ester

The 3-tert-butoxycarbonylamino-cyclopentanecarboxylic acid ethylformate ester was then dissolved in tetrahydroforan (106 mL) and cooled to −65° C. Sodium borohydride (1.91 g, 50.5 mmol) and methanol (3.39 mL) were added and the mixture stirred at −40° C. for 30 min., then at 0° C. for 3 hr. 10% HCl (aq) was added to pH 3 and the mixture was concentrated to half volume. Then it was extracted with ethyl acetate, washed with water, brine, dried over Na₂SO₄, filtered, and concentrated to give (1S,3R)-(3-hydroxymethyl-cyclopentyl)-carbamic acid tert-butyl ester as a white solid (8.65 g, 92%).

ESI MS m/e 216, M+H⁺; ¹H NMR (400 MHz, DMSO-d₆) δ 6.74 (d, J=6.8 Hz, 1 H), 4.46 (bt, J=4.8 Hz, 1 H), 3.70 (m, 1 H), 3.25 (t, J=5.6 Hz, 2 H), 1.92 (m, 2 H), 1.73 (m, 2 H), 1.55 (m, 2 H), 1.38 (s 9 H) 1.05 (m, 1 H).

### Step C: Synthesis of cis-(1S,3R)-[3-(1,3-dioxo-1,3-dihydro-isoindol-2-ylmethyl)-cyclopentyl]-carbamic acid tert-butyl ester

cis-(1S,3R)-(3-Hydroxymethyl-cyclopentyl)-cabamic acid tert-butyl ester (8.65 g, 40.2 mmol), triphenylphosphine (10.54 g, 40.2 mmol), and phthalimide (5.91 g, 40.2 mmol) were dissolved in tetrahydrofuran (128 mL). The mixture was cooled to 0° C. and a solution of diethylazodicarboxylate (6.96 mL, 44.22 mmol) in tetrahydrofuran (30 mL) was added over a period of 1 hr. The mixture stirred at room temperature for 18 hr, concentrated, and purified by silica get chromatography (30% ethyl acetate in hexanes) to give cis-(1S,3R)-[3-(1,3-dioxo-1,3-dihydro-isoindol-2-ylmethyl-cyclopentyl]-carbamic acid tert-butyl ester (9.52 g, 69%) as a solid.

ESI MS m/e 345 M+H⁺; ¹H NMR (400MHz, DMSO-d₆) δ 7.83 (mn, 4 H), 6.84 (dd, J=11.2, 7.6 Hz, 1 H, 3.70 (m, 1 H), 3.54 (m, 2 H), 1.92 (m, 2 H), 1.73 (m, 2 H), 0.55 (m, 2 H), 1.38 (s, 9 H), 1.10 (m, 1 H).

### Step D: Synthesis of cis-(1S,3R)-(3-aminonethyl-cyclopentyl)-carbamic acid tert-butyl ester

The cis-(1S,3R)-[3-(1,3-dioxo-1,3-dihidro-isoindol-2-yl methyl)-cyclopentyl]-carbamic acid tert-butyl ester was suspended in 95% ethanol (143 mL), hydrazine (1.89 mL, 60.3 mmol) was added, and the mixture was heated to reflux temperature (120° C.) for 2.5 hr, then stirred at room temperature for 18 hr. The suspension was concentrated, suspended in 10% NaOH (aq) (182 mL), extracted with dichloromethane, dried over Na₂SO₄, filtered, and concentrated to give cis-(1S,3R)-(3-aminomethyl-cyclopentyl)-carbamic acid tert-butyl ester as a white solid (6.25 g, ˜73%) (crude).

ESI MS m/e 215, M+H⁺; ¹H NMR (400 MHz, DMSO-d₆) δ 6.82 (d, J=6.3 Hz, 1 H), 3.70 (m, 1 H), 1.92 (m, 2 H), 1.75 (m, 2H), 1.73 (m, 2 H), 1.58 (m, 2 H), 1.38 (s, 9 H), 1.30 (m, 2 H), 1.00 (m, 1 H).

### Step E: Synthesis of cis-(1S,3R)-N-(3-amino-cyclopentylmethyl)-3,4-dichlorobenzamide

(cis-(1S,3R)-(3-Aminomethyl-cyclopentyl)-carbamic acid tert-butyl ester (0.050 g, 0.230 mmol), 3,4-dicllorobenzoyl chloride (0.049 g, 0.230 mmol), and diisopropylethylamine (0.10 mL, 0.57 mmol) were combined in dichloromethane (2 mL) and stirred for 18 hr at room temperature. The mixture was concentrated, neutralized with saturated aqueous NaHCO₃, and extracted with dichloromethane. The organic phase was then concentrated to give cis-(1S,3R)-N-(3-amino-cyclopentylmethyl)-3,4-dichloro-benzamide as the crude product.

ES MS m/e 287, M+H⁺; ¹H NMR (400 MHz, DMSO-d₆) δ 8.72 (t, J=5.6 Hz, 1 H), 8.04 (d, J=2.0 Hz, 1 H), 7.78 (d, J=2.0 Hz, 1 H), 7.74 (s, 1 H), 3.40 (m, 2 H), 2.80 (brs, 2 H), 2.15 (m, 1 H), 1.88 (m, 2 H), 1.70 (m, 1 H), 1.58 (m, 2 H), 1.48 (m, 2 H).

### Step F: Synthesis of 3,4-dichloro-N-[(1R,3S)-3-{[4-(dimethylamino)quinazolin-2-yl]amino}-cyclopentyl)methyl]benzamide trifluoroncetate

cis-(1S,3R)-(2-Chloro-quinazolin-4-yl)-dimethyl-amine (0.048 g, 0.23 mmol), N-(3-amino-cyclopentylmethyl)-3,4-dichloro-benzamide (0.23 mmol), diisopropylethylamine (0.061 mL, 0.34 mmol), and isopropanol (1.50 mL) were combined and heated to 160° C. for 40 min. utilizing a Smith synthesizer microwave apparatus. The mixture was then purified by HPLC to give 3,4-dichloro-N-[((1R,3S)-3-{[4-(dimethylamino)quinazolin-2-yl]amino}cyclopentyl)methyl]benzamide trifluoroacelate as a white solid (0.035 g, 26.% over four steps).

ESI MS m/e 458, M+H⁺; ¹H NIR (400 MHz, DMSO-d₆) δ 8.70 (t, J=5.2 Hz, 1 H), 8.20 (brs, 1 H), 8.14 (d, J=8.0 Hz, 1 H), 8.04 (d, J=1.6 Hz, 1 H), 7.80 (d, J=2.0 Hz, 1 H), 7.78 (d, J=2.0 Hz, 1 H), 7.74 (s, 1 H), 7.44 (brs, 1 H), 7.34 (t, J=7.6 Hz, 1 H), 3.29 (t, J=5.2 Hz, 2 H), 2.50 (s, 6 H), 2.24 (m, 1 H), 2.00 (m, 2 H), 1.76 (m, 1 H), 1.65 (m, 2 H), 1.50 (m, 2 H).

### Example 918

### N²-[(1S,3R)-3-({[4-Bromo-2-(trifluoromethoxy)benzyl]amino}methyl)cyclopentyl]-N⁴,N⁴-dimethylquinazoline-2,4-diamine bistrifluoroacetate

### Step A: Synthesis of cis-(1S,3R)-3-[(4-bromo-2-trifluoromethoxy-benzylamino)-methyl]-cyclopentylamine

(3-Aminomethyl)-cyclopentyl)-carbamic acid tert-butyl ester (0.050 g, 0.23 mmol), 4-bromo-2-trifluoromethoxybenzaldehyde (0.063 g, 0.23 mmol), and sodium cyanoborohydride (0.022 g, 0.34 mmol) were combined in methanol (1.00 mL) and stirred at room temperature for 18 hrs. The mixture was concentrated, water (1.00 mL) was added, and it was extracted with dichloromethane. To the organic phase was added trifluoroacetic acid (1.00 mL) and the mixture stirred at room temperature for 18 hrs. The mixture was concentrated, neutralized with saturated aqueous NaHCO₃, extracted with dichloromethane, and concentrated to give (1S,3R)-3-[(4-bromo-2-trifluoromethoxy-benzylamino)-methyl]-cyclopentlamine as the crude product.

ESI MS m/e 367, M+H⁺; ¹H NMR (400 MHz, DMSO-d₆) δ 7.75-7.62 (m, 3 H), 4.58 (s 1 H), 3.77 (s, 2 H), 3.35 (brs, 2 H), 2.48 (m, 2 H), 2.04 (m, 1 H), 1.74 (m, 2 H), 1.38 (m, 2 H), 1.30 (m, 2 H) 0.98 (m, 1 H).

### Step B: Synthesis of N-[(1S,3R)-3-({[4-bromo-2-(trifluoromethoxy)benzyl]amino}methyl)-cyclopentyl]-N⁴,N⁴-dimethylquinazoline-2,4-diamine bistrifluoroacetate

(2-Chloro-quinazolin-4-yl)-dimethyl-amine (0.048 g, 0.23 mmol), (1S,3R)-3-[(4-bromo-2-trifluoromethoxy-benzylamino)-methyl]-cyclopentylamine (0.23 mmol), diisopropylethylamine (0.061 mL. 0.34 mmol), and isopropanol (1.50 mL) were combined and heated to 160° C. for 40 min. utilizing a Smith Synthesizer microwave apparatus. The mixture was then purified by HPLC to give N²-[(1S,3R)-3-({[4-bromo-2-(trifluoronmethoxy)benzyl]amino}methyl)cyclopentyl]-N⁴,N⁴-dimethylquinazoline-2,4-diamine bistrifluoroacetate as a white solid (0.011 g, 62% over four steps).

ESI MS m/e 538 , M+H⁺; ¹H NMR (400 MHz, DMSO-d₆) δ 8.45 (brs, 1 H), 8.14 (d, J 12.0 Hz, 1 H), 7.72 (d, J=2.0 Hz, 1 H), 7.68 (d, J=2.0 Hz, 1 H), 7.63 (s, 1 H), 7.58 (d, J=2.0 Hz, 1 H), 7.44 (brs, 1 H), 7.29 (bt, J=7.6 Hz, 1 H), 4.18 (s, 2 H), 3.40 (s, 2 H), 3.40 (s, 6H) 2.25 (m, 2 H), 1.98 (m, 1 H), 1.82 (m, 2 H), 1.60 (m, 2 H), 1.40 (m, 2 H), 1.22 (m, 1 H).

### Example 919

### N-[(1S,3R)-3-({[4-(Dimethylamino)quinazolin-2-yl]amino}methyl)cyclopentyl]-4-fluorobenzamide trifluoroacetate

### Step A: Synthesis of (1R,3S)-N²-(3-amino-cyclopentylmethyl)-N⁴,N⁴-dimethyl-quinazoline-2,4-diamine

(2-Chloro-quinazolin-4-yl)-dimethyl-amine (0.048 g, 0.23 mmol), (1S,3R)-(3-aminomethyl-cyclopentyl)-carbarnic acid tert-butyl ester (0.050 g, 0.23 mmmol), diisopropylethylamiiie (0.061 mL, 0.34 mmol), and isopropanol (1.50 mL) were combined and heated to 160° C. for 40 min. utilizing a Smith synthesizer microwave apparatus. The mixture was concentrated, dichloromethane (2.00 mL) and trifluoroacetic acid (1.00 mL) wvere added, and the mixture stirred at room temperature for 18 hr. Then it was concentrated, neutralized with saturated aqueous NaHCO₃, extracted with dichloromethane, and concentrated to give (1R,3S)-N²-(3-amino-cyclopentylmethyl)-N′,N′-dmethyl-quinazoline-2,4-diamine as the crude product.

ES MS m/e 286, M+H⁺; ¹H NMR (400 MHz, DMSO-d₆) δ 7.92 (d, J=8.0 Hz, 1 H), 7.53 (t. J=6.0 Hz, 1 H), 7.34 (d, J=7.06 Hz, 1 H), 7.06 (t, J=6.0 Hz, 1 H), 6.78 (brs, 1 H), 3.25 (s, 6 H), 2.28 (m, 2 H), 2.10 (m, 2 H), 1.86 (m, 1 H), 1.75 (m, 2 H), 1.52 (m, 2 H), 1.30 (brs, 2 H), 1.17 (1 H).

### Step B: Synthesis of N-[(1S,3R)-3-({[4-(dimethylamino)quinazolin-2-yl]amino}methyl)-cyclopentyl]-4-fluorobtenzamide trifluoroacetate

cis-(1R,3S)-N²-(3-Amino-cyclopenylmethyl)-N⁴,N⁴-dimethyl-quinazoline-2,4-diamine (0.23 mmol), 4-fluorobenzoyl chloride (0.028 mL, 0.23 mmol), and diisopropylethylamine (0.10 mL, 0.57 mmol) were combined in dichloromethane (2.00 mL) at room temperature and stirred for 18 hrs. The mixture was concentrated, dissolved in methanol, and purified by prep-LCMS to give N-[(1S,3R)-3-({[4-(dimethylamitio)quinazolin-2-yl]amino}methyl)cyclopentyl]-4-fluorobenzamide trifluoroacetate as a white solid (5 mg, 4.1 % over four steps).

ESI MS m/e 408, M+H⁺; ¹H NMR (400 MHz, CD₃OD) ε 8.09 (d, J=8.0 Hz, 1 H), 7.76 (d, J=5.3 Hz, 2 H), 7.74(d, J=5.3Hz,2H),7.66(t,J=8.3Hz,1H),7.) 7.30 (bm,2H),7.07 (t, J=4.9 Hz,1 H), 4.25 (m, 1 H), 3.45 (brs, 6 H), 2.2-5 (m?, 2 H), 2.00 (m, 1 H), 1.70 (m, 2 H), 1.62 (m, 2 H), 1.52 (m, 2 H), 1.26 (m, 1 H).

### Example 920

### N²-({(1R,3S)-3-[(3,4-Difluorobenzyl)amino]cyclopentyl}methyl)-N⁴,N⁴-dimethylquinazoline-2,4-diamine bistrifuoroacetate

### Step A: Synthesis of N²({(1R,3S)-3-[(3,4-difluorobenzyl)amino]cyclopentyl}methyl)-N⁴,N⁴-dimethylquinazoline-2,4-diamine bistrifluoroacetate

(1R,3 S)-N²-(3-Amino-cyclopentylmethyl)-N⁴, N⁴-dimethyl-quinazoline-2,4-diamine (0.23 mmol), 3,4-difluorobenzaldehyde (0.026 mL, 0.23 mmol), and sodium cyanoborohydride (0.022 g, 0.34 mmol) were combined in methanol (1.00 mL) and stirred at room temperature for 18 hr. Water (0.50 mL) was added and the mixture was then purified by prep-LCMS to give N²-({(1R,3S)-3[(3,4-difluorobenzyl)amino]cyclopentyl}methyl)-N⁴,N⁴-dimethylquinazoline-2,4-diamine bistrifluoroacetate as a white solid (0.011 g, 7.4% over four steps). ESI MS m/e 412, M+H⁺; ¹H NMR (400 MHz, DMSO-d₆) δ 8.83 (brs, 1 H), 8.12 (d, J=7.7 Hz, 1 H), 7.73 (t, J=4.9 Hz, 1 H), 7.55 (t, J=9.7 Hz, 1 H), 7.50 (q, J=8.9 Hz, 1 H), 7.31 (m, 2 H), 4.09 (brs, 1 H), 3.42 (brs, 6 H), 3.36 (brs, 1 H), 2.18 (m, 2 H), 1.95 (m, 1 H), 1.69 (m, 2 H), 1.42 (m, 2 H), 1.26 (m, 2 H), 1.18 (brs, 2 H) 0.81 (m, 1 H).

### Example 921

### cis-N-(1,3-Dimethoxybenzyl)-4-{[4-(dimethylamino)quinazolin-2-yl]amino}cyclohexanecarboxamide

### Step A: Synthesis of cis-4-amino-cyclohexanecarboxylic acid ethyl ester hydrochloride

To a suspension of cis-aminocyclohexane-4-carboxylic acid (1.5 g, 10 mmol) in EtOH (15 mL) was added concentrated HCl (1.5 mL). The reaction was stirred for 2 hr at 72° C. Removal of the volatile solvent under a vacuum gave cis-4-amino-cyclohexanecarboxylic acid ethyl ester hydrochloride (1.7 g, 96%) as a white power, which was used directly to the next reaction without a further purification.

ESI IS m/e 172 M+H⁺; ¹H NMR (400 MHz, DMSO-d₆) δ 4.43 (brs, 2 H), 4.05 (q, J=7.2 Hz, 2 H), 3.02 (brs, 1 H), 2.48 (m, 1 H), 1.943 (m, 2 H), 1.76 (m, 2 H), 1.43-1.57 (m, 4 H), 1.17 (t, J=7.2 Hz, 3 H).

### Step B: Synthesis of cis-4-(4-dimethylamino-quinazolin-2-ylamino)-cyclohexane carboxylic acid ethyl ester

The reaction was done in seven vials. Each vial contains 2-chloro-4-N,N-dimethylamino quinazoline (0.26 g, 1.25 nimol), cis-(4-ethoxycai-bonyl)aminocyclohexane hydrochloride (0.25 g, 1 eq.), DIEA (0.45 mL, 2 eq.), and IPA (2 mL). The vials were heated at 155° C. for 1 hr using a Smith microwave synthesizer. The vial contents were combined and concentrated. The residue was purified on silica gel column using CH₂Cl₂/MeOH (100:0 to 85:15) to give cis-4(4-dimethylamino-quinazolin-2-ylamino)-cyclohexane carboxylic acid ethyl ester (2.2 g, 76%) as pale yellow oil.

ESI MS m/e 343 M+H⁺; ¹H NMR (400 MHz, MeOD) δ 8.17 (d, J=8.0 Hz, 1 H), 7.76 (t,J=8.0 Hz, 1 H), 7.40 (brs, 1 H), 7.40 (t, J=8.0 Hz, 1H), 4.660 (brs, 1 H), 4.16 (q, J=6.8 Hz, 2 H), 3.53 (s, 6 H), 2.59 (m, 1 H), 1.97-1.63 (m, 8 H), 1.27 (t, J=6.8 Hz, 3 H), the

### Step C: Syntlieis of cis-4-(4-dimethylamino-quinaolin-2-ylamino)-cyclohexane carboxylic acid

A suspension of cis-4-(4-dimethylamino-quinazolin-2-ylamino)-cyclohexane carboxylic acid ethyl ester (0.35 g, 1 mmol) in 4N-HCl (10 mL) was stirred at 82° C. for 2 h. During the reaction, the heterogenous solution turned to be a clear solution, and then the precipitate was formed. The solid was filtered, washed with cold water several times, and dried to give 0.29 g (90 %) of cis-4(4-dimethylamino-quinazolin-2-ylamino)-cyclohexane carboxylic acid as a white solid.

ESI MS m/e 315 M+H⁺; ¹H NMR (400 MHz, DMSO-d₆) δ 12.3 (brs, 1 H), 8.13 (d, J=7.6 Hz, 2 H), 7.74 (t, J=7.6 Hz, 1 H), 7.37 (brs, 1 H), 7.36 (t, J=7.6 Hz, 1 H), 4.05 (brs, 1 H), 3.32 (s, 6 H), 2.42 (brs, 1 H), 1.82-1.68 (m, 8 H).

### Step D: Synthesis of cis-N-(2,3-dimethoxybenzyl)-4-{[4-(dimethylamino)quinazolin-2-yl]-amino}cyclohexanecarboxamide

To a suspension of the acid (25 mg, 0.08 mol) and the 2,3 dimethoxy benzyl amine (13 mg, 0.08 mmol) in DCM (3 mL) was added HATU (33 mg, 0.088 mmol), and followed Et₃N (4 drops). The reaction was stirred overnight at room temperature under an inert atmosphere. After removal of the volatile solvent, the crude product was purified by column chromatography (silica gel, DCM/MeOH=100:0 to 90:10) to give cis-N-(2,3-dimethoxybenzyl)-4-{[4-(dimethylamino)quinazolin-2-yl]amino}cyclohexanecarboxamide (8 mg, 21%).

ESI MS m/e 464 M+H+; ¹H NMR (400 MHz, CDCl₃) δ 8.34 (brs, 1 H), 7.89 (d, J=8.4 Hz, 1 H), 7.57 (t, J=6.8 Hz, 1 H), 7.33 (d, J=8.0 Hz, 1 H), 7.23 (t, J=7.6 Hz, 1 H), 7.07 (brs, 1 H), 6.95 (t, J=7.6 Hz, 1 H), 6.89 (d, J=8.0 Hz, 1 H), 6.76 (d, J=8.0Hz, 1 H), 4.56 (d, J=5.6Hz, 2 H), 4.30 (m, 1 H), 3.33 (s, 3 H), 3.80 (s, 3 H), 3.48 (s, 6 H), 2.35 (m, 1 H), 2.05-1.82 (m, 6 H), 1.66 (mn, 2 H),

### Example 92

### cis-N-(2,4-Difluorobenzyl)-4-{[4-(dimethylamino)quinazolin-2-yl]amino}cyclohexanecarbozamide

### Step A: Synthesis of cis-N-(2,4-difluorobenzyl)-4-{[4-(dimethylamino)quinazolin-2-yl]amino}-cyclohexanecarboxamide

Using a similar procedure as described in step D of Example 921, the title compound was obtained.

ESI MS m/e 440 M+H⁺; ¹H NMR (400 MHz, CDCl₃) δ 7.92 (d. J=8.0 Hz, 1 H), 7.61 (t, J=8.0 Hz, 1 H), 7.28 (m, 3 H), 7.17 (brs, 1 H), 6.82 (brs, 1 H), 6.76 (t, J=8.0 Hz, 1 H), 6.67 (t, J=8.0 Hz, 1 H), 4.41 (d, J=6.0 Hz, 2 H), 4.31 (brs, 1 H), 3.51 (s, 6 H), 2.39 (m, 1 H), 1.96-1.66 (m, 8 H),

### Example 923

### cis-4-{[4-(Dimethylamino)quinazolin-2-yl]amino}-N-(2,3-dimethylbenzyl)cyclohexane carboxamide

### Step A: Synthesis of cis-4-({[4-(dimethylamino)quinazolin-2-yl] amino}-N-(2,3-dimethylbenzyl)-cyclohexanecarbosamide

Using a similar procedure as described in step D of Example 921 , the title compound ,was obtained.

ESI MS m/e 432 M+H⁺; ¹H NMR (400 MHz, CDCl₃) δ 7.91 (d, J=8.4 Hz, 1 H), 7.58 (t, J=7.6 Hz, 1 H), 7.27 (t, J=7.6 Hz, 1 H), 7.13 (d, J=7.6 Hz, 1 H), 7.06 (m, 1 H), 6.99 (d, J=4.4 Hz, 2 H), 6.90 (brs, 1 H), 6.45 (brs, 1 H), 4.41 (d, J=6.0 Hz, 2 H), 4.25 (brs, 1 H), 3.50 (s, 6 H), 2.41 (m, 1 H), 2.21 (s, 3 H), 2.14 (s, 3H), 1.96-1.72 (m, 8 H).

### Example 924

### cis-N-(2-Bromobenzyl)-4-{[4-(dimethylaminoquinazolin-2-yl]amino}cyclohexane-carboxamide

Step A: Synthesis of cis-N-(2-bromobenzyl)-4-{[4-(dimethlamino)quinazolin-2-yl]amino}-cyclohexanecarboxamide

Using a similar procedure as described in step D of Example 921, the title compound was obtained.

ESI MS m/e 482 M+H⁺; ¹H NMR (400 MHz, CDCl₃) S 7.91 (d, J=8,4 Hz, 1 H), 7.62 (t, J=8.0 Hz, 1 H), 7.43 (d, J=7.6 Hz, 1 H), 7.31-7.21 (m, 4 H), 7.05 (t, J=7.2 Hz, 1 H), 6.82 (brs, 1 H), 6.59 (brs, 1 H), 4.48 (d, J=6.0 Hz, 2 H), 4.30 (brs, 1 H), 3.52 (s, 6 H), 2.41 (m, 1 H), 1.97-1.64 (m, 8 H).

### Example 925

### cis-N-(2,4-Dichlorobenzyl)-4-{[4-(dimethylamino)quinazolin-2-yl]amino}cyclohexanecarboxamide

### Step A: Synthesis of cis-N-(2,4-dichlorobenzyl)-4-{[4-(dimethylaminoquinazolin-2-yl]amino}-cyclohexanecarboxamide

Using a similar procedure as described in step D of Example 921, the title compound was obtained.

ESI MS m/e 472 M+H⁺; ¹H NMR (400 MHz, CDCl₃) δ 7.91 (d, J=S.0 Hz, 1 H), 7.61 (t, J=8.0 Hz, 1 H), 7.28 (t, J=7.6 Hz, 1 H), 7.25-7.19 (m, 3 H), 7.12 (d, J=8.0 Hz, 1 H), 6.98 (brs, 1 H), 6.83 (brs, 1 H), 4.43 (d, J=6.0 Hz, 2 H), 4.31 (brs, 1 H), 3.S2 (s, 6 H), 2.42 (m, 1 H), 1.96-1.67 (m, S H).

### Example 926

### cis-N-(2.3-Dichlorobenzyl)-4-{[4-(dimethylamino)quinazolin-2-yl]amino}cyclohexanecarboxamide

### Step A: -Synthesis of cis-N-(2,3-dichlorobenzyl)-4-{[4-(dimethylamino)quinazolin-2-yl]amino}-cyclohexanecarboxamide

Using a similar procedure as described in step D of Eample 921, the title compound was obtained.

ESI MS m/e 472 M+H⁺; ¹NMR (400 MHz, CDCl₃) δ 8.17 (d, J=8.4 Hz, 1 H), 7.75 (t. J=7.6 Hz, 1 H), 7.45-7.37 (m, 3 H), 7.30-7.24 (m, 2 H), 4.4S (s, 2 H), 4.26 (brs, 1 H), 3.54 (s, 6 H), 2.49 (m, 1 H), 1.99-1.77 (m, 8 H).

### Example 927

### cis-N-(2,5-Dichlorobenzyl)-4-{[4-(dimethylamino)quinazolin-2-yl]amino}cyclohexanecarboxamide

Step A: Synthesis of cis-N-(2,5-dichlorobenzyl)-4-{[4-(dmethylamino)quinizolin-2-ylamino}-cyclohexanecarboxamide

Using a similar procedure as described in step D of Example 921, the title compound was obtained.

ESI MS m/e 472 M+H⁺; ¹H NMR (400 MHz, CDCl₃) 5 7.90 (d, J=8.4 Hz, 1 H), 7.66 (t, J=7.6 Hz, 1 H), 7.58 (brs, 1 H), 7.39 (d, J=8.0 Hz, 1 H), 7.31-7.19 (n, 3 H), 7.10 (d, J=8.4 Hz, 1 H), 7.01 (brs, 1 H), 4.48 (d, J=6.0 Hz, 2 H), 4.30 (brs, 1 H), 3.53 (s, 6 H), 2.42 (m, 1 H), 1.98-1.90 (m, 6 H). 1.63 (m, 2 H).

### Example 928

### cis-N-(2-Chlorobenzyl)-4-{[4-(dimethylamino)quinazolin-2-yl]amino}cyclolhexanecarboxamide

### Step A: Synthesis of cis-N-(2-chlorobenzyl)-4-{[4-(dimethylamino)quinazolin-2-yl]amino}-cyclohexanecarboxamide

Using a similar procedure as described in step D of Example 921, the title compound was obtained.

ESI MS m/e 438 M+H⁺; ¹H NMR (400 MHz, CDCl₃) δ 7.90 (d, J=8.4 Hz, 1 H), 7.60 (t, J=8.0 Hz, 1 4), 7.31-7.09 (m, 6 H), 6.77 (d, J=6.8 Hz, 1 H), 6.66 (brs, 1 H), 4.49 (d, J=6.0 Hz, 2 H), 4.27 (brs, 1 H), 3.51 (s, 6 H), 2.43 (m, 1 H), 1.95-1.68 (m, 8 H).

### Example 929

### cis-N-(3-Chlorobenzyl)-4-{14-(dimethylamino)quinazolin-2-yl]amino}cyclohexanecarboxamide

### Step A: Synthesis of cis-N-(3-chlorobenzyl)-4-{[4-(dimethylamino)quinazolin-2-yl]amino}-cyclohexanecarboxamide

Using a similar procedure as descnbed in step D of Example 921, the title compound was obtained.

ESI MS m/e 438 M+H⁺; ¹H NMR (400 MHz, CDCl₃) δ 8.13 (d, J=8.0 Hz, 1 H), 7.72 (t, J=7.6 Hz, 1 H), 7.49-7.19 (m, 6 H), 4.35 (s, 2 H), 4.23 (brs, 1 H), 3.51 (s, 6 H), 2.44 (m, 1 H), 2.01-1.74 (m, 8 H).

### Example 930

### cis-4-{[4-(Dimetlylamino)quinazolin-2-yl]amino}-N-(3-methoxybenzyl)cyclohexanecarboxamide

### Step A: Synthesis of cis-4-{[4-(dimethylamino)quinazolin-2-yl]amino}-N-(3-methoxybenzyl)-cyclohexanecarboxamide

Using a similar procedure as described in step D of Example 921, the title compound was obtained.

ESI MS m/e 434M+H⁺; ¹H NMR (400 MHz, CDCl₃) δ 7.90 (d, J=8.4 Hz, 1 H), 7.62 (t, J=8.0 Hz, 1 H), 7.29 (t, J=8.0 Hz, 1 H), 7.22 (m, 1 H), 7.14 (t, J=8.0 Hz, 1 H), 6.85-6.78 (m, 3 H), 6.71 (d, J=8.0 Hz, 1 H), 6.63 (brs, 1 H), 4.38 (d, J=6.0 Hz, 2 H), 4.29 (brs, 1 H), 3.51 (s, 6 H), 2.40 (m, 1 H), 1.95-1.66 (m, 8 H).

### Example 931

### cis-4-{[4-(Dimethylamino)quinazolin-2-yl]amino}-N-(4-methylbenzyl)cyclohexanecarboxamide

### Step A: Synthesis of cis-4-{[4-(dimethylamino)quinazolin-2-yl]amino}-N-(4-methylbenzyl)-cyclohexanecarboxamide

Using a similar procedure as described in step D of Example 921, the title compound was obtained.

ESI MS m/e 418 M+H⁻; ¹H NMR (400 MHz, CDCl₃) 6 9.80 (brs, 1 H), 7.90 (d, J=8.4 Hz, 1 H), 7.63 (t, J=8.0Hz, 1 H), 7.29 (t, J=8.0 Hz, 1 H), 7.16 (d, J=8.0 Hz, 2 H), 7.06 (d, J=8.0Hz, 2 H), 6.77 (d, J=7.2 Hz, 1 H), 6.48 (brs, 1 H), 4.37 (d, J=5.6 Hz, 2 H), 4.29 (brs, 1 H), 3.52 (s, 6 H), 2.37 (m, 1 H), 2.27 (s, 3 H), 1.96-1.62 (m, 8 H).

### Example 932

### cis-N-[3,5-Bis(trifluoromethyl)benzyl]-4-{[4-(dimethylaminoquinazolin-2-yl]amino}cyclohexanecarboxamide

### Step A: Synthesis of cis-N-[3,5-bis(trifluoromethyl)benzyl]-4-{[4-(dimethylamino)quinazolin-2-yl]amino}cyclohexanecarboxamide

Using a similar procedure as described in step D of Example 921, the title compound was obtained.

ESI MS m/e 540 M+H⁺; ¹H NMR (400 MHz, CDCl₃) δ 8.91 (brs, 1 H), 8.22 (brs, 1 H), 7.SS (d, J=7.6 Hz, 1 H), 7.78 (s, 2 H), 7.68 (s, 1 H), 7.62 (t, J=8.0 Hz, 1 H), 7.40 (d, J=8.0 Hz, 1 H), 7.24 (t, J=7.6Hz, H), 4.55 (d, J=5.6Hz, 2 H), 4:8 (S(m, 1 H), 3.49 (s, 6 H), 2.44 (m, 1 H), 2.19 (m, 2H), 1.95 (m, 4 H), 1.62 (m, 2 H).

### Example 933

### cis-N-(2,4-Dimethoxybenzyl)-4-{[4-(dimethylamino)quinazolin-2-yl]amino}cyclohexanecarboxamide

### Step A: Synthesis of cis-N-(2,4-dimethoxybenzyl)-4-{[4-(dimethylaminoquinazolin-2-yl]amino}cyclohexanecarboxamide

Using a similar procedure as described in step D of Example 921, the title compound was obtained.

ESI MS m/e 464 M+H⁺; ¹H NMR (400 MHz, CDCl₃) δ 8.53 (brs, 1 H), 7.88 (d, J=7.6 Hz, 1 H), 7.60 (t, J=8.0 Hz, 1 H), 7.40 (d, J=7.6 Hz, 1 H), 7.23 (t, J=7.6 Hz, 1 H), 7.16 (d, J=8.4 Hz, 1 H), 6.83 (brs, 1 H), 6.3S (m, 2 H)4.37 (d, J=6.0 Hz, 2 H), 4.2O ((m, 1 H),3.82 (s, 3 H),3.75(s. 3 H),3.48 (s, 6 H), 2.32 (m, 1 H), 2.09-1.2 (m, 6 H), 1.66 (m, 2 H).

### Example 934

### cis-N-(3,4-Dimethoxybenzyl)-4-{[4-(dimethylamino)quinazolin-2-yl]amino}cyclohexanecarboxamide

Step A: Synthesis of cis-N-(3,4-dimethoxybenzyl)-4-{[4-(dimethylamino)quinazolin-2-yl]-amino}cyclohexanecarboxamide

Using a similar procedure as described in step D of Example 921, the title compound was obtained.

ESI MS m/e 464 M+H⁺; ¹H NMR (400 MHz, CDCl₃) δ 8.74 (brs, 1 H, 7.87 (d, J=8.0 Hz, 1 H), 7.61 (t, J=7.6 Hz, 1 H), 7.41 (d, J=8.0 Hz, 2 H), 7.23 (t, J=7.2 Hz, 1 H), 6.91 (m, 2 H), 6.76 (d, J=8.0Hz, 1 H), 4.37 (d, J=6.0 Hz, 2H), 4.36 (m, 1 H), 3.87 (s, 3 H), 3.81 (s, 3 H), 3.48 (s, 6 H), 2.37 (m, 1 H), 2.09 (m, 2 H), 1.83 (m, 4 H), 1.63 (m, 2 H).

### Example 935

### cis-N-(3,5-Dimethoxybenzyl)-4-{[4-(dimethylamino)quinazolin-2-yl]amino}cyclohexanecarboxamide

### Step A: Synthesis of cis-N-(3,5-dimethoxybenzyl)-4-{[4-(dimethylamino)quinazolin-2-yl]-amino}cyclohexanecarboxanide

Using a similar procedure as described in step D of Example 921, the title compound was obtained.

ESI MS m/e 464 M+H⁺; ¹H NMR (400 MHz, CDCl₃) δ 8.32 (brs, 1 H), 7.88 (d, J=7.6 Hz, 1 H), 7.59 (t, J=7.6 Hz, 1 H), 7.34 (d, J=8.0 Hz, 1 H), 7.23 (t, J=7.6 Hz, 2 H), 6.46 (d, J=2.0 Hz, 2 H), 6.25 (t, J=2.0 Hz, 1 H), 4.36 (d, J=6.0 Hz, 2 H), 4.34 (bm,1 H), 3.73 (s, 6 H), 3.48 (s, 6 H), 2.39 (m, 1 H), 2.06-1.83 (m, 6 H), 1.65 (m, 2 H).

### Example 936

### cis-4-{[4-(Dimethylamino)quinazolin-2-yl]amino})-N-(4-hydroxy-3-methoxybenzyl)-cyclohexanecarboxamide

### Step A: Synthesis of cis-4-{[4-(dimethylamino)quinazolin-2-yl]amino}-N-(4-hydroxy-3-methoxybenzyl)cyclohexanecarboxamide

Using a similar procedure as described in step D of Example 921, the title compound was obtained.

ESI MS m/e 450 M+H⁺; ¹H NMR (400 MHz, CDCl₃) 8 8.02 (brs, 1 H), 7.88 (d, J=7.6 Hz, 1 H), 7.63 (t, J=8.0 Hz, 1 H), 7.36 (d, J=8.0 Hz, 1 H), 7.26 (t, J=8.0 Hz, 1 H), 7.04 (brs, 1 H), 6.90 (d, J=1.2 Hz, 1 H), 6.79 (m, 2 H), 4.33 (d, J=6.0 Hz, 3 H), 3.87 (s, 3 H), 3.55 (s, 1 H), 3.50 (s, 6 H), 2.37 (m, 1 H), 1.93-1.83 (m, 6 H), 1.65 (m, 2 H).

### Example 937

### cis-4-{[4-(Dimethylamino)quinazolin-2-yl]amino}-N-(3,4,5-trimethoxybenzyl)cyclohexanecarboxamide

### Step A: Synthesis of cis-4-{[4-(dimethylamino)quinazolin-2-yl]amino}-N-(3,4,5-trimethoxybenzyl)cyclohexanecarboxamide

Using a similar procedure as described in step D of Example 921, the title compound was obtained.

ESI MS m/e 494 M+H⁺; ¹H NMR (400 MHz, CDCl₃) δ 7.90 (d, J=8.4 Hz, 1 H), 7.66 (t, J=8.0 Hz, 1 H), 7.30 (m, 2 H), 6.78 (d, J=7.2 Hz, 1 H), 6.56 (s, 3H), 4.34 (d, J=6.0 Hz, 3 H), 3.82 (s, 6 H), 3.78 (s, 3 H), 3.52 (s, 6 H), 2.38 (m, 1 H), 1.97-1.62 (m, 8 H).

### Example 938

### cis-4-{[4-(Dimetlylamino)quinazolin-2-yl]amino}-N-(2,4,6-trimethoxybenzyl)cyclohexanecarboxamide

### Step A; Synthesis of cis-4-{[4-(dimethylamino)quinazolin-2-yl]amino}-N-(2,4,6-trimethoxybenzyl)cyclohexanecarboxamide

Using a similar procedure as described in step D of Example 921, the title compound was obtained.

ESI MS m/e 494 M+H⁺; ¹H NMR (400 MHz, CDCl₃) 8 8.48 (brs, 1 H), 7.87 (d, J=8.4Hz, 1 H), 7.60 (t, J=8.0 Hz, 1 H), 7.44 (d, J=6.8 Hz, 1 H), 7.22 (t, J=8.0 Hz, 1 H), 6.28 (brs, 1 H), 6.09 (s, 2 H), 4.45 (d, J=5.2 Hz, 2 H), 4.20 (brs, 1 H), 3.83 (s, 6 H), 3.78 (s, 3 H), 3.48 (s, 6 H), 2.26 (m, 1 H), 1.97-1.65 (m, 8 H).

### Example 939

### cis-N-(1 ,3-Benzodioxol-5-ylmethyl)-4-{[4-(dimethylamino)quinazolin-2-yl]amino}cyclohexanecarboxamide

### Step A: Synthesis of cis-N-(1,3-benzodioxol-5-ylmethyl)-4-{[4-(dimethylamino)quinazolin-2-yl]amino}cyclohexanecarboxamide

Using a similar procedure as described in step D of Example 921, the title compound was obtained.

ESI MS m/e 448 M+H⁺; ¹H NMR (400 MHz, CDCl₃) δ 8.52 (brs, 1 H), 7.89 (d, J=7.6 Hz, 1 H), 7.59 (t, J=8.0 Hz, 1 H), 7.39 (brs, 1 H), 7.44 (d, J=8.0 Hz, 1 H), 7.23 (t, J=7.6 Hz, 1 H), 6.79 (s, 1 H), 6.75 (d, J=8.0 Hz, 1 H), 6.66 (d, J=8.0 Hz, 1 H), 5.84 (s, 2 H), 4.35 (m, 1 H), 4.32 (d, J=6.0 Hz, 2 H), 3.48 (s, 6 H), 2.37 (m, 1 H), 2.05 (m, 2 H), 1.87 (m, 4 H), 1.63 (m, 2 H).

### Example 940

### cis-4-{[4-(Dimethylamino)quinazolin-2-yl]amino}-N-(2,2-diphenylethyl)cyclohexanecarboxamide

### Step A: Syuthesis of cis-4-{[4-(dimethylamino)quinazolin-2-yl]amino}-N-(2,2-diphenylethyl)-cyclohexanecarboxamide

Using a similar procedure as described in step D of Example 921, the title compound was obtained.

ESI MS m/e 494 M+H⁺; ¹H NMR (400 MHz, CDCl₃) δ 7.84 (d, J=8.4 Hz, 1 H), 7.56 (t, J=7.6 Hz, 1 H), 7.46 (d, J=8.4 Hz, 1 H), 7.27-7.15 (m, 13 H), 4.38 (brs, 1 H), 4.27 (brs, 1 H), 3.91 (dd, J=8.0, 6.0 Hz, 2 H), 3.39 (s, 6 H), 2.16 (m, 1 H), 1.79 (m, 4 H), 1.60 (m, 4 H).

### Example 941

### cis-4-{[4-(Dimethylamino)quinazolin-2-yl]amino}-N-(1,2,3,4-tetrahydronaphthalen-1-yl)cyclohexanecarboxamide

### Step A: Synthesis of cis-4-{[4-(dimethylamino)quinazolin-2-yl]amino}-N-(1,2,3,4-tetrahydronaphthalen-1-yl)cyclohexanecarboxamide

Using a similar procedure as described in step D of Example 921, the title compound was obtained.

ESI MS mi/e 444 M+H⁺; ¹H NMR (400 MHz, CDCl₃) δ 7.89 (d, J=7.6 Hz, 1 H), 7.63 (t, J=7.6 Hz, 1 H), 7.34-7.03 (m, 6 H), 6.80 (brs, 1 H), 6.09 (d, J=8.4 Hz, 1 H), 5.15 (q, J=6.8 Hz, 1 H), 4.27 (brs, 1 H), 3.52 (s, 6 H), 2.83 (m, 1 H), 2.70 (m, 1 H), 2.36 (m, 1 H), 2.04-1.72 (m, 12 H).

### Example 942

### cis-N-(2,3-Dihydro-1H-inden-2-yl)-4-({[4-(dimethylamino)quinazolin-2-yl]amino}cyclohexanecarboxamide

### Step A: Synthesis of cis-N-(2,3-dihydro-1H-inden-2-yl)-4-{[4-(dimethylamino)quinazolin-2-yl]amino}cyclohexanecarboxamide

Using a similar procedure as described in step D of Example 921, the title compound was obtained.

ESI MS ni/e 430 M+H⁺; ¹H NMR (400 MHz, CDCl₃) δ 7.91 (d, J=8.4 Hz, 1 H), 7.63 (t, J=8.0 Hz, 1 H), 7.28 (m, 2 H), 7.15 (m, 2 H). 7.09 (m, 2 H), 6.83 (d, J=6.83 Hz, 1 H), 6.34 (d, J=6.8 Hz, 1 H), 4.63 (m, 1 H), 4.29 (brs, 1 H), 3.51 (s, 6 H), 3.24 (m, 2 H), 2.97 (m, ¹H), 2.33 (m, 1 H), 1.97-1.68 (m, 8 H).

### Example 943

### cis-4-{[4-(Dimethylamino)quinazolin-2-yl]amino}-N-[2-(5-methoxy-1H-indol-3-yl)ethyl]cyclohexanecarboxamide

### Step A: Synthesis of cis-4-{[4-(dimethylaminoquinazolin-2-yl]amino}-N-[2-(5-methoxy-1H-indol-3-yl)ethyl]cyclohexanecarboxamid

Using a similar procedure as described in step D of Example 921, the title compound was obtained.

ESI MS m/e 487 M+H⁺; ¹H NMR (400 MHz, CDCl₃) δ 8.32 (brs, 1 H), 7.89 (d, J=8.4 Hz 1 H), 7.53 (t, J=8.0 Hz, 1 H), 7.40 (d, J=8.4 Hz, 1 H), 7.22 (d, J=S.4 Hz, 1 H), 7.10 (t, J=7.6 Hz, 1 H), 7.02 (s, 2 H), 6.81 (dd, J=8.8, 2.0 Hz, 1 H), 5.80 (brs, 1 H), 4.21 (brs, 1 H), 3.84 (s, 3 H), 3.59 (q, J=6.0 Hz, 2 H), 3.34 (s, 6 H), 2.95 (t, J=6.4 Hz, H), 2.19 (m, 1 H), 1.85 (m, 2 H), 1.72-1.63 (m, 6 H).

### Example 944

### cis-4-{[4-(Dimethylamino)quinazolin-2-yl]amino}-N-[(1R)-1-(4-nitrophenyl)ethyl]-cyclohexanecarboxamide

### Step A: Synthesis of cis-4-{[4-(dimethylamino)quinazolin-2-yl]amino}-N-[(1R)-1-(4-nitrophenyl)ethyl]cyclohexanecarboxamide

Using a similar procedure as described in step D of Example 921, the title compound was obtained.

ESI MS m/e 463 M+H⁺; ¹H NMR (400 MHz, CDCl₃) δ 8.13 (d, J=8.8 Hz, 2 H), 7.88 (d, J=7.6 Hz, 2 H), 7.63 (m, 3 H), 7.43 (d, J=7.6 Hz, 1 H), 7.24 (t, J=7.6 Hz, 1 H), 5.13 (m, 1 H), 4.44 (m, 1 H), 3.48 (s, 6 H), 2.35 (m, 1 H), 2.16 (m, 2 H), 1.88 (m, 4 H), 1.76 (m, 1 H), 1.63 (m, 1 H), 1.61 (d, J=7.2 Hz, 3 H).

### Example 945

### cis-4-{[4-(Dimethylamino)quinazolin-2-yl]amino}-N-[(1S)-1-(4-nitrophenylethyl)-cyclohexanecarboxamide

### Step A: Synthesis of cis-4-{[4-(dimethylamino)quinazolin-2-yl]amino}-N-[(1S)-1-(4-nitrophenyl)ethyl]cyclohexanecarboxamide

Using a similar procedure as described in step D of Example 921, the title compound was obtained.

ESI MS m/e 463 M+H⁺; ¹H NMR (400 MHz, CDCl₃) δ 8.13 (d, J=8.8 Hz, 2 H), 7.88 (d, J=7.6 Hz, 2 H), 7.63 (m, H), 7.43 (d, J=7.6 Hz, 1 H), 7.24 (t, J=7.6 Hz, 1 H). 5.13 (m, 1 H), 4.45 (m, J=7.2Hz,3 H).

### Example 946

### cis-4-{[4-(Dimethylamino)quinazolin-2-yl]amino}-N-[(1R,2S)-2-hydroxy-2,3-dihydro-1H-inden-1-yl]cyclohexanecarboxamide

### Step A: Synthesis of cis-4-{[4-(dimethylamino)quinazolin-2-yl]amino}-N-[(1R,2S)-2-hydroxy-2,3-dihydro-1H-inden-1-yl]cyclohexanecarboxamide

Using a similar procedure as described in step D of Example 921, the title compound was obtained.

ESI MS m/e 446 M+H⁺; ¹H NMR (400 MHz, CDCl₃) δ 7.85 (d, J=8.0 Hz, 1 H), 7.59 (t, J=7.6 Hz, 1 H), 7.39 (d, J=8.0 Hz, 1 H), 7.29-7.15 (m, 6 H), 7.12 (brs, 1 H), 5.39 (m, 1 H), 4.69 (brs, 1 H), 4.39 (m, 1 H), 4.23 (brs, 1 H), 3.47 (s, 6 H), 3.12 (m, 2 H), 2.47 (m, 1 H), 2.16-1.88 (m, 6 H), 1.67 (m, 2 H).

### Example 947

### cis-4-{[4-(Dimethylamino)quinazolin-2-yl]amino}-N-[(1S,2R)-2-hydroxy-2,3-dihydro-1H-inden-1-yl]cyclohexanecarboxamide

### Step A: Synthesis of cis-4-{[4-(dimethylamino)quinazolin-2-yl]amino}-N-[(1S,2R)-2-hydroxy-2,3-dihydro-1H-inden-1-ylcyclohexanecarboxamide

Using a similar procedure as described in step D of Example 921, the title compound was obtained.

ESI MS m/e 446 M+H⁺; ¹H NMR (400 MHz, CDCl₃) δ 8.73 (brs, 1 H), 7.86 (d, J=8.0 Hz, 1 H), 7.59 (t, J=8.0 Hz: 1 H), 7.38 (d, J=8.4 Hz, 1 H), 7.28-7.15 (m, 5 H), 7.11 (brs, 1 H), 5.38 (m, 1 H), 4.69(m, 1 H),4.39 (m, 1 H), 4.28 (brs, 1 H),3.48(s,6H), 3.12 (m, 2H), 2.47 (m, 1 H), 2.16-1.88 (m, 6 H), 1.67 (m, 2 H).

### Example 948

### cis-4-(4-Dimethylamino-quinazolin-2-ylamino)-cyclohexanecrboxylic acid (trans 2-phenylcyclopropyl)-amide

### Step A: Synthesis of cis-4-(4-dimethylamino-quinazolin-2-ylamino)-cyclohexanecarboxylic acid (2-phenylcyclopropyl)-amide

Using a similar procedure as described in step D of Example 921, the title compound was obtained.

ESI MS m/e 430 M+H⁺; ¹HMR (400 MHz, CDCl₃) δ 7.89 (d, J=7.6 Hz, 1 H), 7.64 (t, J=7.6 Hz, 1 H), 7.28 (t, J=8.0 Hz, 2 H), 7.09-7.01 (m, 6 H), 6.65 (brs, 1 H), 4.28 (brs, 1 H), 3.50 (s, 6 H), 2.92 (brs, 1 H), 2.40 (brs, 1 H), 2.13 (m, 1 H), 1.95-1.68 (m, 8 H), 1.31 (m, 1 H), 1.17 (m, 1 H).

### Example 949

### cis-4-{[4-(Dimethylamino)quinazolin-2-yl]amino}-N-[(1S)-1-(4-methylphenyl)ethyl]-cyclohexanecarboxamide trifluoroacetate

### Step A: Synthesis of cis-4-{[4-(dimethylamino)quinazolin-2-yl]amino}-N-[(1S)-1-(4-nethylphenyl)ethyl]cyclohexanecarboxantide trifluoroacetate

Using a similar procedure as described in step D of Example 921, the product was purified by prep HPLC to give the title compound.

ESI IMS m/e 432 M+H⁺; ¹H NMR (400 MHz, DMSO-d₆) δ 11.72 (brs, 1 H), 8.10 (d, J=8.0 Hz, 1 H), 8.08 (brs, 1 H), 7.90 (brs, 1 H), 7.71 (t, J=8.0 Hz, 1 H), 7.37 (brs, 1 H), 7.30 (t, J=8.0 Hz, 1 H), 7.11 (d, J=8.0 Hz, 2 H), 7.04 (d, J=8.0 Hz, 2 H), 4.81 (m, 1 H), 4.10 (brs, 1 H), 3.36 (s, 6 H), 2.26 (brs, 1 H), 2.19 (s, 3 H), 1.80-1.51 (m, 8 H), 1.24 (d, J=7.2 Hz, 3 H).

### Example 950

### cis-4-{[4-(Dimethylamino)quinazolin-2-yl]amino}-N-(1R)-1-(1-naphthyl)ethyl]cyclohexanecarboxamide trifluoroacetate

### Step A: Synthesis of cis-4-{[4-(dimethylamino)quinazolin-2-yl]amino}-N-[(1R)-1-(1-naphthyl) ethyl]cyclohexanecarboxamide trifluoroacetate.

Using a similar procedure as described in step D of Example 921, the product was purified by prep HPLC to give the title compound.

ESI MS m/e 468 M+H⁺; ¹H NMR (400 MHz, DMSO-d₆) δ 11.8 (brs, 1 H), 8.30 (d, J=7.6 Hz, 1 H), 8.10 (d, J=8.0 Hz, 1 H), 8.03 (d, J=8.0 Hz, 1 H), 7.94 (brs, 1 H), 7.87 (d, J=8.4 Hz, 1 H), 7.75 (d, J=8.0 Hz, 1 H), 7.70 (t, J=7.6 Hz, 1 H), 7.4.-7.40 (m, 4 H), 7.36 (brs, 1 H), 7.29 (t, J=7.6 Hz, 1 H), 5.64 (m, 1 H), 4.09 (brs, 1 H), 3.40 (s, 6 H), 2.28 (brs, 1 H), 1.84-1.50 (m, 8 H), 1.42 (d, J=7.0 Hz, 3 H).

### Example 951

### (-cis-4-{[4-(Dimethylamino)quinazolin-2-yl]amino}-N-[3-(trifluoromethyl)benzyl]cyclohexanecarboxamide

### Step A: Synthesis of cis-4-(4-dimethylamino-quinazolin-2-ylanmino)-cyclohexane carbonyl chloride

To a suspension of cis-4-(4-dimethylamino-quinazolin-2-ylamino)-cyclohexane carboxylic acid (0.34 g, 1.0 mmol) in CH₂Cl₂ (20 mL) was added 2M-oxalyl chloride (7.4 mL, 1.3 eq.) in CH₂Cl₂ under an inert atmosphere. The reaction was stirred for 18 hr at room temperature. The reaction changed to a clear solution. Removal of the volatile solvent gave the crude cis-4-(4-dimethylamino-quinazolin-2-ylamino)-cyclohexane carbonyl chloride (0.35 g, 97%), which was directly used to the next reaction without a further purification (When the acid chloride reacted with EtOH, the formation of 343 M+H⁺ of the ethyl ester) was observed by LC-MS).

### Step B: Synthesis of cis-4-{[4-(dimethylamino)quinazolin-2-yl]amino}-N-[3-(trifluoromethyl)-benzyl]cyclohexanecarboxamide

To a solution of the acid chloride (24 mg, 0.07 mmol), obtained from Step A, in DCM (3 mL) was added the 3-trifluoromethylbenzyl amine (13 mg, 0.07 mmol) and followed DIEA (3 drops). After stirring overnight at room temperature, the reaction was quenched and purified using column chromatography (silica gel, DCM/MeOH=100:0 to 90:10) to give cis-4-{[4-(dimethylamino)quinazolin-2-yl]amino}-N-[3-(trifluoromethyl)benzyl]cyclohexanecarboxamide (18 mg, 53 %).

ESI MS m/e 472 M+H⁺; ¹H NMR (400 MHz, CDCl₃) δ 7.84 (d, J=8.4 Hz, 1 H), 7.57-7.3S (m, 7 H), 7.12 (t, J=7.2 Hz, 1 H), 4.50 (d, J=6.0 Hz, 2 H), 4.35 (brs, 1 H), 3.37 (s, 6 H), 2.36 (m, 1 H), 2.06-1.82 (m, 6 H), 1.66 (m, 2 H).

### Example 952

### cis-4-{[4-(Dimethylamino)quinazolin-2-yl]amino}-N-(3-methoxyphenyl)cyclohexanecarboxamide

### Step A: Synthesis of cis-4-{[4-(dimethylamino)quinazolin-2-yl]amino}-N-(3-methoxyphenyl)-cyclohexanecarboxamide

Using a similar procedure as described in step B of Example 951, the title compound was obtained.

ESI MS m/e 420 M+H⁺; ¹H NMR (400 MHz, CDCl₃) δ 7.35 (d, J=7.6 Hz, 1H), 7.56 (m, 2 H), 7.45 (d, J=7.6 Hz, 1 H), 7.37 (brs, 1 H), 7.17 (m, 2 H), 6.59 (d, J=8.0 Hz, 1 H) 4.42 (brs, 1 H), 3.81 (s, 3 H), 3.44 (s, 6 H), 2.45 (m, 1 H), 2.18 (m, 2 H), 1.94 (m, 4 H), 1.67 (m, 2 H).

### Example 953

### cis-4-{[4-(Dimethylamino)quinazolin-2-yl]amino}-N-(2-methoxybenzyl)cyclohexanecarboxamide

### Step A: Synthesis of cis-4-{[4-(dimethylamino)quinazolin-2-yl]amino}-N-(2-methoxybenzyl)-cyclohexanecarboxamide

Using a similar procedure as described in step B of Example 951, the title compound was obtained.

ESI MS m/e 434 M+H⁺; ¹HMR (400 MHz, CDCl₃) δ 7.83 (d, J=8.0 Hz, 1 H), 7.56 (t, J=7.2 Hz, 1 H), 7.45 (d, J=8.0 Hz, 1 H), 7.27-7.14 (m, 4 H), 6.88 (t, J=7.6 Hz, 1 H), 6.83 (d, J=8.0 Hz, 1 H), 4.45 (d, J=5.6 Hz, 2 H), 4.31 (brs, 1 H), 3.86 (s, 3 H), 3.40 (s, 6 H), 2.31 (m, 1 H), 2.02-1.82 (m, 6 H), 1.66 (m, 2 H).

### Example 954

### cis-4-{[4-(Dinetlylamino)quinazolin-2-yl]amino)-N-(3-iodobenzyl)cyclohexanecarboxamide cyclohexanecarboxamide

Using a similar procedure as described in step B of Example 951, the title compound was obtained.

ESI MS m/e 530 M+H⁺; ¹H NMR (400 MHz, CDCl₃) δ 7.86 (d, J=8.4 Hz, 1 H), 7.66 (s, 1 H), 7.65 (d, J=7.6 Hz, 1 H), 7.54 (m H), 7.44 (d, J=8.0 Hz, 1 H, 7.32 (d, J=8.0 Hz, 1 H), 7.25 (d, J=S.0 Hz, 1 H), 7.19 (t, J=7.6 Hz, 1 H), 7.03 (m, H), 4.40 (d, =6.0 Hz, 3 H), 3.44 (s, 6H), 2.38 (m, 1 H), 2.06 (m, 2 H), 1.89 (m, 4 H), 1.63 (m, 2 H).

### Example 955

### cis-N-(3,5-Dichlorobenzyl)-4-{[4-(dimethylamino)quinazolin-2-yl]amino}cyclohexanecarboxamide

### Step A: Synthesis of cis-N-(3,5-dichlorobenzyl)-4-{[4-(dimethylamino)q uinazolin-2-yl]amino}cyclohexanecarboxamide

Using a similar procedure as described in step B of Example 951, the title compound was obtained.

ESI MS m/e 472 M+H⁺; ¹H NMR (400 MHz, CDCl₃) δ 7.84 (d, J=8.0 Hz, 1 H), 7.57 (t, J=7.6 Hz, 1 H), 7.44 (d, J=8.0 Hz, 1 H), 7.19 (bm, 4 H), 4.40 (d, J=6.0 Hz, 3 H) 3.42 (s, 6 H), 2.3S (m, 1 H), ) 2.05 (m, 2 H), 1.89 (m, 4 H), 1.65 (m, 2 H).

### Example 956

### cis-4-{[4-(Dimethylamino)quinazolin-2-yl]amino}-N-[4-(trifluoromethoxy)benzyl]-cyclohexane carboxamide

### Step A: Synthesis of cis-4-{[4-(dimethylamino)quinazolin-2-yl]amino}-N-[4-(trifluoromethoxy)benzyl]cyclohexanecarboxamide

Using a similar procedure as described in step B of Example 951, the title compound was obtained.

ESI MS m/e 488 M+H⁺; ¹H NMR (400 MHz, CDCl₃) δ 7.84 (d, J=8.4 Hz, 1 H), 7.58 (t, J=8.0 Hz, 1 H), 7.43 (d, J=8.0 Hz, 1 H), 7.37-7.31 (m, 3 H), 7.19-7.11 (m, 4 H), 4.44 (d, =6.0 Hz, 2 H), 4.48 (brs, 1 H), 3.42 (s, 6 H), 2.36 (m, 1 H), 2.05 (m, 2 H), 1.89 (m, 4 H), 1.64 (m, 2 H).

### Example 957

### cis-N-(4-Bromobenzyl)-4-{14-(dimethylamino)quinazolin-1-yl]amino}cyclohexanecarboxamide

### Step A: Synthesis of cis-N-(4-bronobenl)-4-{[4-(dimethylamino)quinazolin-2-yl]amino}-cyclohexanecarboxamide

Using a similar procedure as described in step B of Example 951, the title compound was obtained.

ESI MS m/e 488 M+H⁺; ¹H NMR (400 MHz, CDCl₃) δ 7.87 (d, J=8.4 Hz, 1 H), 7.75 (brs, 1 H), 7.62 (t, J=7.6 Hz, 1 H), 7.44 (brs, 1 H), 7.42 (d, J=8.0 Hz, 2 H), 7.38 (d, J=8.4 Hz, 1 H), 7.24 (m, 1 H),7.17(d, J=8.0 Hz, 2H),4.40(d,J=6.0Hz,3H),3.47(s,6H),2.38 (m, 1 H),2.10(m,2H), 1.87 (m,4 H), 1.61 (m,2 H).

### Example 958

### cis-4-{[4-(Dimethylamino)quinazolin-2-yl]amino}-N-(4-methoxybenzyl)cyclohexanecarboxamide

### Step A: Synthesis of cis-4-{[4-(dimethylamino)quinazolin-2-yl]amino}-N-(4-methoxybenzly)-cyclohexanecarboxamide

Using a similar procedure as described in step B of Example 951, the title compound was obtained.

ESI MS m/e 434 M+H⁺; ¹H NMR (4.00 MHz, CDCl₃) δ 7.85 (d, J=8.4 Hz, 1 H), 7.60 (t, J=7.6 Hz, 1 H), 7.45 (d, J=8.4 Hz, 1 H), 7.28 (d, J=8.8 Hz, 2 H), 7.19 (t, J=7.6 Hz, 1 H). 6.82 (d, J=8.4 Hz, 2 H), 4.39 (d, J=6.0 Hz, 2H), 3.45 (s, 6 H) 2.35 (m, 1 H), 2.05 (m, 2 H), 1.87 (m, 4 H) 1.62 (m, 2 H).

### Example 959

### cis-N-(2,4-Dimethoxyphenyl)-4-{[4-(dimethylamino)quinazolin-2-yl]amino}cyclohexanecarboxamide

### Step A: Synthesis of cis-N-(2,4-dimethoxyphenyl)-4-{[4-(dmethylamino)quinazolin-2-yl]amino}cyclohexaaecarboxamide

Using a similar procedure as described in step B of Example 951, the title compound was obtained.

ESI MS m/e 450 M+H⁺; ¹H NMR (400 MHz, CDCl₃) δ 8.16 (d, J=8.8 Hz, 1 H), 7.83 (d, J=8.0 Hz, 2 H), 7.55 (m, 1 H), 7.49 (m, 1 H), 7.13 (brs, 1 H), 6.45 (s, 1 H), 6.43 (m, 1 H), 4.27 (brs, 1 H), 3.89 (s, 3 H), 3.77 (s, 3 H), 3.39 (s, 6 H), 2.42 (m, 1 H), 2.04-1.96 (m, 6 H), 1.75 (m, 2 H).

### Example 960

### cis-N-(3,5-Dichlorophenyl)-4-{[4-(dixethylamino)quinizolin-2-yl]amino}cyclohexanecarboxamide

### Step A: Synthesis of cis-N-(3,5-dichlorophenyl)-4-{[4-(dimethylamino)quinazolin-2-yl]-amino}cyclohexanecarboxamide

Using a similar procedure as described in step B of Example 951, the title compound was obtained.

ESI MS m/e 458 M+H⁺; ¹H NMR (400 MHz, CDCl₃) δ 7.86 (m, 3 H), 7.60 (t, J=7.6 Hz, 1 H), 7.45 (d J=8.4 Hz, 1 H), 7.20 (t, J=7.6 Hz, 1 H), 7.01 (s, 1 H), 4.44 (brs, 1 H), 3.45 (s, 6 H) 2.47 (m, 1 H), 2.18 (m, 2 H), 1.96 (m, 4 H), 1.66 (m, 2 H).

### Example 961

### cis-4-{[4-(Dimethylamino)quinazolin-2-yl]amino}-N-(3-iodophenyl)cyclohexanecarboxamide

### Step A: Synthesis of cis-4-{[4-(dimethylamino)quinazolin-2-yl]amino}-N-(3-iodophenyl)-cyclohexanecarboxamide

Using a sinuilar procedure as described in step B of Example 951, the title compound was obtained.

ESI MS m/e 516 M+H⁺; ¹H NMR(400 MHz, CDCl₃) δ 8.15(s, 1 H), 7.83 (d,J=8.0 Hz, 1 H), 7.63 (d, J=7.2 Hz, 1 H), 7.54 (t, J=7.6 Hz, 1 H), 7.44 (d, J=8.0Hz, 1 H), 7.38 (d, J=7.6 Hz, 1 H), 7.11 (t, J=7.6 Hz, 1 H), 7.00 (t, J=7.6 Hz, 1 H), 4.37 (brs, 1 H), 3.36 (s, 6 H), 2.42 (m, 1 H), 2.09-1.66 (m, 8 H).

### Example 962

### cis-4-{[4-(Dimethylamino)quinazolin-2-yl]amino}-N-(2-fuoro-4-nitrophenyt)cyclohexanecarboxamide

### Step A: Synthesis of cis-4-{[4-(dimethylaminoquinazolin-2-yl[amino}-N-(2-fluoro-4-nitrophenyl)cyclohexanecarboxamide

Using a similar procedure as described in step B of Example 951, the title compound was obtained.

ESI MS m/e 453 M+H⁺; ¹H NMR (400 MHz, CDCl₃) δ 8.08 (brs, 1H) 7.96 (m, 1 H) 7.83 (d, J=8.0 Hz, 1 H), 7.56 (t J=7.6 Hz, 1 H), 7.44 (d, J=8.0 Hz, 1 H), 7.20 (m, 1 H), 7.14 (t, J=7.6 Hz, 1 H), 4.38 (brs, 1 H), 3.40 (s, 6 H), 2.54 (m, 1 H), 2.17 (m, 2 H), 1.97 (m, 4 H), 1.74 (m, 2 H).

### Example 963

### cis-4-{[4-(Dimethylamino)quinazolin-2-yl]amino}-N-(2-methoxydibenzo[b,d]furan-3-yl)cyclohexanecarboxamide trifluoroacetate

### Step A: Synthesis of cis-4-{[4-(dimethylamino)quinazolin-2-yl]amino}-N-(2-methoxydibenzo[b,d]furan-3-yl)cyclohexanecarboxamide trifluoroacetate

Using a similar procedure as described in step B of Example 951, the product was purified by prep HPLC to give the the compound.

ESI MS m/e 510 M+H⁺; ¹H NMR (400 MHz, DMSO-d₆) δ 11.8 (brs, 1 H), 9.19 (s, 1 H), 8.38 (s, 1 H), 8.12 (d, J=7.6 Hz, 1 H), 8.01 (d, J=8.0 Hz, 1 H), 8.00 (brs, 1 H), 7.74 (s, 1 H), 7.72 (m, 1 H), 7.57 (d, J=8.0Hz, 1 H), 7.38 (t, J=8.4 Hz, 2 H), 7.29 (m, 2 H), 4.17 (brs, 1 H), 3.92 (s, 3 H), 3.39 (s, 6 H), 2.73 (brs, 1 H), 1.88-1.64 (m, 8 H).

### Example 964

### (cis-4-{[4-(Dimethylamino)quinazolin-2-yl]amino}cyclohexyl)methyl 3,5-dichlorobenzoate

### Step A: Synthesis of cis-(4-hydroxymethyl-cyclohexyl)-carbamic acid tert-butyl ester

To a suspension of cis-4-(tert-butoxycarbonylamino)-cyclohexanecarboxlic acid (15.0 g, 61.7 mmol) in CH₂Cl₂ (140 mL) at −65° C. was added trietlhylamine (13 mL, 2.7 eq.) and a solution of ethyl chloroformate (6 mL) in CH₂Cl₂ (20 mL). The reaction was stirred for 60 min. at 0° C., and acidified (pH =˜3) with IN-HCl. The mixture was extracted with CH₂Cl, (2×70 mL), and the combined organic layers wvere washed with sat. aqueous Na₂CO₃ (1×60 mL), water (2×80 mL), and brine (1×80 mL) and dried over MgSO₄ filtered, and concentrated to give cis-(4-hydroxymethyl-cyclohexyl)-carbamic acid tert-butyl ester as a colorless oil. To a solution of the crude oil in THF (150 mL) at −65° C. were added NaBH₄ (2.7 g, 73 mmol) and MeOH (4.8 mL). The reaction was stirred for 3o min. at -40° C., and stirred for an additional 3 hr at 0° C. The reaction was acidified with 1N-HCl, removed a half volume of solvent, and extracted with EtOAc (3×100 mL). The combined organic layer was washed with water (3×80 mL) and brine (1×100 mL), dried with MgSO₄, filtered, and concentrated to give the product (11.5 g, 82 %) as a white solid. ESI MS m/e 230 M+H⁺; ¹H NMR (400 MHz, CDCl₃) δ 4.69 (brs, 1 H), 3.78 (brs, 1 H), 3.50 (d, J=6.4 Hz, 2 H), 2.19 (brs, 1 H), 1.10-1.55 (m, 7 H), 1.44 (s, 9 H), 1.25 (m, 2 H).

### Step B: Synthesis of cis-(4-amino-cyclohexyl)-methanol hydrochloride

To a solution of cis-(4-hydroxymethyl-cyclohexyl)-carbamic acid tert-butyl ester (0.5g, 2.1 mmol) in EtOAc (15 mL) was added 4NI-HCl (10 mL) at room temperature, The reaction was stirred for 1.5 h at room temperature and concentrated to give a crude compound, which was washed with CH₂Cl₂ (the product was not soluble in CH₂Cl₂) to remove organic impurities to give 0.25 g (89 %) of cis-(4-amino-cyclohexyl)-methanol hydrochloride as a white solid.

ESI MS m/e 130 M+H⁺; ¹H NMR (400 MHz, CD₃OD) δ 3.51 (d, J=7.2 Hz, 2 H), 3.31 (brs, 1 H), 1.81-1.57 (m, 9 H).

### Step C: Synthesis of cis-[4-(4-dmethylamino-quinazolin-2-ylamino)-cyclohexyl]-methanol

A vial contains 2-chloro-4-N,N-dimethylamino quinazoline (0.31 g, 1.5 mmol), cis-(4-amino-cyclolhexyl)-methaiiol hydrochloride (0.25 g, 1 eq.), DIEA (0.55 mL), and IPA (2 mL). The vial was heated at 155° C. for 1 h using a Smith itmicrowave synthesizer. The vial contents was diluted with DCM, washed with diluted HCl and water, and concentrated. The residue was purified on silica gel column using CH₂Cl₂ and MeOH (100:0 to 80:20) to give cis-[4-(4-dimethylamino-quinazolin-2-ylamino)-cyclohexyl)-methanol (0.16 g, 28 %) as a pale yellow solid.

ES1 MS m/e 301 M+H⁺; ¹H NMR (400 MHz, CDCl₃) δ 8.69 (brs, 1 H), 7.86 (d, J=8.8 Hz. 1H), 7.59 (t, J=8.4 Hz, 1 H), 7.51 (d, J=8.4 Hz, 1 H), 7.21 (t, J=8.0 Hz, 1 H). 4.26 (brs, 1 H), 3.57 (s, 2 H), 3.49 (s, 6 H), 1.92 (m, 3 H), 1.65 (m, 6 H).

### Step D: Synthesis of (cis-4-{[4-(dimethylamino)quinazolin-2-yl]amino}cyclohexyl)methyl 3,5-dichloroobenzoate

To a solution of cis-[4-(4-dimethylamino-quinazolin-2-yl]amino}-cyclohexyl]-methanol (25 mg, 0.08 mmol) in DCM (3 mL) was added 3,5-dichlorobenzoyl chloride (17 mg, 0.08 mmol) and followed DIEA (3 drops). The reaction was stirred overnight at room temperature under an inert atmosphere. The reaction was diluted with DCM, washed with 1N-HCl and water, and concentrated. The product was purified by coluinn chromatography (DCM/MeOH=100:0 to 90:10) to give (cis-4-{[4-(dimethylamino)quinazolin-2-yl]amino}cyclohexyl)methyl 3,5-dichlorobenzoate (15 mg, 38%).

ESI MS m/e 473 M+H⁺; ¹H NMR (400 MHz, CDCl₃) δ 7.88 (s, 2 H), 7.80 (d, J=8.4 Hz, 1 H), 7.51 (m, 2 H), 7.46 (t, J=8.0 Hz, 1 H), 7.06 (t, J=7.6 Hz, 1 H), 4.27 (m, 1 H), 4.22 (d, J=7.2 Hz, 2 H), 3.32 (s, 6 H), 1.92 (m, 3 H), 1.72 (m, 4 H), 1.54 (m, 2 H).

### Example 965

### (cis-4-{[-(Dimethylamino)quinazolin-2-yl]amino}cyclohexyl)methyl 3-methoxybenzoate

### Step A: Synthesis of (cis-4-{[4-(dimethylamino)quinazolin-2-yl]amino}cyclohexyl)methtl 3-methozybenzoate

Using a similar procedure as described in step D of Example 964, the title compound was obtained.

ESI MS m/e 435 M+H⁺; ¹HNMR (400 MHz, CDCl₃) δ 7.81 (d, J=8.4 Hz, 1 H), 7.64-7.53 (m, 4 H), 7.30 (t, J=7.6 Hz, 1 H), 7.11 (t, J=7.6 Hz, 1 H), 7.06 (d, J=8.4 Hz, 1 H), 4.26 (brs, 1 H), 4.25 (d, J=6.8 Hz, 2 H), 3.84 (s, 3 H), 3.39 (s, 6 H), 1.97 (m, 3 H), 1.72 (m, 6 H).

### Example 966

### (cis-4-{[4-(Dimethylamino)quinazolin-2-yl]amino}cyclohexyl)methyl 3-bromobenzoate

### Step A: Synthesis of (cis-4-{[4-(dimethylamino)quinazolin-2-yl]amino}cyclohexyl)methyl 3-bromobenzoate.

Using a similar procedure as described in step D of Example 964, the title compound was obtained.

ESI MS m/e 483 M+H⁻; ¹H NMR (400 MHz, CDCl₃) δ 8.15 (s, 1 H), 7.96 (d, J=7.2 Hz, 1 H), 7.80 (d, J=8.0 Hz, 1 H), 7.65 (d, J=7.6 Hz, 1 H), 7.50 (s, 2 H), 7.29 (t, J=7.6 Hz, 1 H), 7.04 (m, 1 H), 4.27 (brs, 1 H), 4.23 (d, J=6.8Hz, 2 H), 3.31 (s, 6 H), 1.93 (m, 3 H), 1.72 (m, 4 H, 1.56 (m, 2 H).

### Example 967

### (cis-4-{[4-(Dimethylamino)quinazolin-2-yl]amino}cyclohexyl)methyl 3,4-difuorobenzoate

### Step A: Synthesis of (cis-4-{[4-(dimethylamino)quinazolin-2-yl]amino}cyclohexyl)methyl 3,4-difluorobenzoate

Using a similar procedure as described in step D of Example 964, the title compound was obtained.

ESI MS m/e 441 M+H⁺; ¹H NMR (400 MHz, CDCl₃) δ 7.81 (m, 3 H), 7.48 (m, 2 H), 7.22 (m, 1 H), 7.04 (t, J=7.6 Hz, 1 H), 4.27 (brs, 1 H). 4.21 (d, J=7.2 Hz, 2 H), 3.31 (s, 6 H), 1.92 (m, 3 H), 1.72 (m, 4 H), 1.55 (m, 2 H).

### Example 968

### 3,4-Dimethoxybenzyl cis-4-{[4-(dimethylamino)quinazolin-2-yl]amino}cyclohexanecarboxylate

### Step A: Synthesis of 3,4-dimethoxybenzyl cis-4-{[4-(dimethylamino)quinazolin-2-yl]amino}cyclohexanecarboxylate

To a solution of the acid chloride (24 mg, 0.07 mmol) in DCM (3 mL) was added 3,4-dimethoxybenzyl alcohol (12 mg, 0.07 mmol) and followed DIEA (3 drops). After stirring overnight at room temperature, the reaction was quenched and purified using column chromatography (silica gel, DCM/MeOH=100:0 to 90:10) to give 3,4-dimethoxybenzyl cis-4-{[4-(dimethylamino)quinazolin-2-yl]amino}cyclohexanecarboxylate (12 mg, 36%).

ESI MS m/e 465 M+H⁺; ¹H NMR (400 MHz, CDCl₃) δ 7.79 (d, J=8.0 Hz, 1 H), 7.49 (t, J=7.6 Hz, 1 H), 7.44 (d, J=7.6 Hz, 1 H), 7.04 (t, J=7.2 Hz, 1 H), 6.91 (d, J=8.0 Hz, 1 H), 6.86 (s, 1 H), 6.84 (t, J=8.0 Hz, 1 H), 5.05 (s, 2 H), 4.11 (brs, 1 H), 3.88 (s, 3 H), 3.87 (s, 3 H), 3.30 (s, 6 H), 2.51 (m, 1 H), 1.97 (m, 2 H), 1.78 (m, 6 H).

### Example 969

### 4-(Trifluoromethoxy)benzyl cis-4-{[4-(dimethylamino)quinazolin-2-yl]amino}cyclohexanecarboxylate

### Step A: Synthesis of 4-(trifluoromethoxy)benzyl cis-4-(14-(dimethylamino)quinazolin-2-yl]amino}cyclohexanecarboxylate

Using a similar procedure as described in step A of Example 968 , the title compound was obtained.

ESI MS m/e 489 M+H⁺; ¹H NMR (400 MHz, CDCl₃) δ 7.84 (d, J=8.0 Hz, 1 H), 7.57 (t, J=7.6 Hz, 1 H), 7.49 (d, J=8.0 Hz, 1 H), 7.39 (d, J=8.4 Hz, 2 H), 7.20 (d, J=8.4 Hz, 2 H), 7.16 (brs, 1 H), 5.12 (s, 2 H), 4.08 (brs, 1 H), 3.42 (s, 6 H), 2.52 (m, 1 H), 2.05 (m, 2 H), 1.79 (m, 6 H).

### Example 970

### 3,5-Dimethoxybenzyl cis-4-{[4-(dimethylamino)quinazolin-2-yl]amino}cyclohexanecarboxylate

### Step A: Synthesis of 3,5-dimethoxybenzyl cis-4-{[4-(dimethylamino)quinazolin-2-yl]amino}-cyclobexanecarboxylate

Using a similar procedure as described in step A of Example 968, the title compound was obtained.

ESI MS m/e 465 M+H⁺; ¹H NMR (400 MHz, CDCl₃) δ 7.81 (d, J=8.4 Hz, 1 H), 7.51 (t, J=7.6 Hz, 1 H), 7.43 (d, J=8.0 Hz, 1 H), 7.08 (t, J=7.6Hz, 1 H), 6.47 (d, J=2.4 Hz,2H), 6.38 (t, J=2/4 Hz, 1 H), 5.05 (s, 2 H), 4.11 (brs, 1 H), 3.77 (s, 6 H), 3.36 (s, 6 H), 2.54 (m 1 H), 2.02 (m, 2 H), 1.79 (m, 6 H).

### Example 971

### 3,4,5-Trimethoxybenzyl cis-4-{[4-(dimethylamino)quinazolin-2-yl]amino}cyclohexanecarboxylate

### Step A: Synthesis of 3,4,5-trimethoxybenzyl cis-4-{[4-(dimethylamino)quinazolin-2-yl]amino}cyclohexanecarboxylate

Usinig a similar procedure as described in step A of Example 968, the title compound was obtained.

ESI MS m/e 495 M+H⁺; ¹H NMR (400 MHz, CDCl₃) δ 7.79 (d, J=8.0 Hz, 1 H), 7.49 (t, J=7.2 Hz, 1 H). 7.43 (d, J=7.6 Hz, 1 H), 7.04 (t, J=7.6 Hz, 1 H), 6.56 (s, 2 H), 5.05 (s, 2 H), 4.11 (brs, 1 H), 3.85 (s, 6 H), 3.83 (s,3 H), 3.29 (s, 6 H), 2.53 (m, 1 H), 2.00 (m, 2 H), 1.79 (m, 6 H).

### Example 972

### 2,3,4-Trimethoxybenzyl cis-4-{[4-(dimethylamino)quinazolin-2-yl]amino}cyclohexanecarboxylate

### Step A: Synthesis of 2,3,4-trimethoxybenzyl cis-4-{[4-(dimetlylamino)quinazolin-2-yl]amino}cyclohexanecarboxylate

Using a similar procedure as described in step A of Example 968, the title compound was obtained.

ESI MS m/e 495 M+H⁺; ¹H NNR (400 MHz, CDCl₃) δ 7.79 (d, J=8.0 Hz, 1 H), 7.49 (t, J=7.6 Hz, 1 I), 7.44 (d, J=7.6 Hz, 1 H), 7.05 (m, 1 H), 7.02 (d, J=8.4 Hz, 1 H), 6.45 (d, J=8.4 Hz, 1 H), 5.09 (s, 2 H), 4.10 (brs, 1 H), 3.90 (s, 3 H), 3.86 (s, 3 H), 3.85 (s, 3 H), 3.30 (s, 6 H), 2.49 (m 1 H), 2.00 (m, 2 H), 1.77 (m, 6 H).

### Example 973

### 1-(2-Naphthyl)ethyl cis-4-{[4-(dimethylamino)quinazolin-2-yl]amino}cyclohexanecarboxylate

### Step A: Synthesis of 1-(2-naphthy)ethyl cis-4-{[4-(dimethylamino)quinazolin-2-yl]amino}-cyclohexanecarboxylate

Using a similar procedure as described in step A of Example 968, the title compound was obtained.

ESI MS m/e 469 M+H⁺; ¹H NMR (400 MHz, CDCl₃) δ 7.85 (m, 5 H), 7.45 (m, 5 H), 7.04 (d, J=7.6 Hz, 1 H), 6.05 (q, J=6.4 Hz, 1 H), 4.11 (brs, 1 H), 3.28 (s, 6 H), 2.52 (m, 1 H), 2.01 (m, 2 H), 1.78 (m, 6 H), 1.62 (d, J=6.4 Hz, 3 H).

### Example 974

### 3-[(Cyclopropylcarbonyl)amino]-N-(cis-4-{[4-(dimethylamino)quinazolin-2-yl]amino}cyclohexyl)benzamide

### Step A: Synthesis of cis-N-(4-amino-cyclobenzyl)-3-nitrobenzamide trifluoroacetate

To a suspension of cis-(4-amino-cyclohexyl)-carbamic acid tert-butyl ester (1.1 g, 5.2 mmol) in DCM (20 mL) was added 3-nitrobenzoyl chloride (0.96 g, 5.2 mmol) and followed catalytic amount of DIEA (0.1 mL). The reaction was stirred overnight at room temperature, diluted with DCM, washed with 1N-HCl and water, and concentrated. The crude product was preliminary purified by a short pad of silica gel with DCMI/MeOH (100:0 to 90:10). The product was contaminated with impurity having a very close rf value with the product. A solution of this crude compound (1.2 g, 3.2 mmol) in DCM/TFA (16 mL=10/6) was stirred for 2 hr at room temperature. After removal of the volatile solvent, the solid residue was suspended in hexane, filtered, and dried to give 1.0 g (83%) of cis-N-(4-amino-cyclohexyl)-3-nitrobenzamide trifluoroacetate.

ESI MS m/e 264 M+H⁺¹H NMR (400 MHz, DMSO-d₆) δ 8.64 (t, J=2.0 Hz, 1 H), 8.49 (d, J=4.8 Hz, 1 H), S.37 (ddd, J=8.0, 2.0, 0.8 Hz, 1 H), S.27 (d, J=8.0 Hz, 1 H), 7.81 (brs, 2 H), 7.75 (t, J=8.0 Hz, 1 H), 3.90 (m, 1 H), 3.15 (brs, 1 H), 2.51 (m, 1 H), 1.91 (m, 2 H), 1.76-1.64 (m, 6 H).

### Step B: Synthesis of cis-N-[4-(4-dimethylamino-quinazolin-2-ylamino)-cyclohexyl]-3-nitrobenzanlide

A suspension of 2-chloro-4-N,N-ditnethylamino quinazoline (0.3 g, 1.4 mmol) and cis-N-(4-amino-cyclohexyl)-3-nitrobenzamide trifluoroacetate (0.5 g, 1.35 mmol) in IPA (2.5 mL) and DIEA (0.7 mL) was reacted for 2 hr at 160° C. in a Smith synthesizer. Over 90% conversion was observed by LC-MS. The reaction was quenched and purified by column chromatography (silica gel, DCM/MeOH=100:0 to 85:15) to give 0.45 g (80% of cis-N-[4-(4-dimethylamio-quinazolin-2-ylamino)-cyclohexyl]-3-nitrobenzamide.

ESI MS m/e 435 M+H⁺; ¹H NMR (400 MHz, CDCl₃) δ 9.04 (d, J=7.6 Hz, 1 H). 8.73 (t, J=2 Hz, 1 H) 8.28 (d, J=8.4 Hz, 1 H) 8.18 (d, J=8.0 Hz, 1 H), 7.88 (d, J=7.2 Hz, 1 H), 7.62 (m, 2 H), 7.49 (d, J=7.6 Hz, 1 H), 7.25 (m, 1 H), 7.16 (d, J=8.4 Hz, 1 H), 4.38 (m, 1 H), 4.18 (m, 1 H), 3.51 (s, 6 H), 1.99-1.93 (m, 6 H), 1.78 (m, 2 H).

### Step C: Synthesis of 3-amino-cis-N-[4-(4-dimethylamine-quinazolin-2-ylamino)-cyclohexyl]-benzamide

A heterogenous solution of cis-N-[4-(4-dimethylamino-quinazolin-2-ylamino)-cyclohexyl]-3-nitrobenzamide (0.85 g, 1.9 mmol) and 10% Pd/C (100 mg) in EtOH (20 mL) was stirred overnight under H. at room temperature. LC-MS confirmed 100% conversion of the stating material. The reaction was filtered throughi a pad of celite. After removal of the volatile solvent, the residue was purified from a short pad of silica gel (DCM/MeOH=100:0 to 80:20) to give 0.48 g (62%) of 3-amino-cis-N-[4-(4-diethylamino-quinazolin-2-ylamino)-cyclohexyl]-benzamide as the desired product.

ESI MS m/e 405 M+H⁺; ¹H NMR (400 MHz, CDCl₃) δ 9.42 (brs, 1 H), 7.89 (d, J=8.0 Hz, 1 H), 7.62 (m, 2 H), 7.26-7.1 7 (m, 4 H), 6.79 (m, 1 H), 6.72 (d, J=8.4 Hz, 1 H), 4.36 (brs, 1 H), 4.18 (m, 1 H), 3.51 (s, 6 H), 1.94-1.78 (m, 8 H).

### Step D: Synthesis of 3-[(cyclopropylcarbonyl)amino]-N-(cis-4-{[4-(dimethylamino)-quinazolin-2-yl]amino}cyclohexyl)benzamide

To a solution of 3-amino-cis-N-[4-(4-dimethylamino-quinazolin-2-ylamino)-cyclohexyl]-benzamide (25 mg, 0.06mmol) in DCM (3 mL) was added cyclopropanecarbonyl chloride (6 mg, 0.06 mmol) and followed DIEA (catalytic, 3 drops). After stirring overnight at room temperature, the reaction was quenched and purified from prep-HPLC [15 to 95% of CH₃CN (5%TFA)/H₂O (5% TFA)] to give 12 mg (33%) of 3-[(cyclopropylcarbonyl)amino]-N-(cis4-{[4-(dimethylamino)quinazolin-2-yl]amino}cyclohexyl)benzaniide.

ESI MS m/e 473 M+H⁺; ¹H NMR (400 MHz, DMSD-d₆) δ 12.1 (brs, 1 H), 10.2 (s, 1 H), 8.2 (d, J=8.0 Hz, 2 H), 7.94 (brs, 1 H), 7.93 (s, 1 H), 7.74-7.67 (m, 2 H), 7.42 (d, J=7.8 Hz, 2 H), 7.31 (m, 2 H), 4.01 (brs, 1 H), 3.83 (brs, 1 H), 3.42 (s, 6 H), 1.83-1.68 (m, 8 H), 1.00 (m, 2 H), 0.93 (m, 2 H).

### Example 975

### N-[cis-4-{[4-(Dimethylamino)quinazolin-2-yl]amino}cyclohexyl)methyl]-3-[(2,2-dimethylpropanoyl)amino]bezamide

### Step A: Synthesis of {cis-4-[(3-nitro-benzoylamino)-methyl]-cyclohexyl}-carbamic acid tert-butyl ester

cis-(4-Aminomethyl-cyclohexyl)-carbamic acid tert-butyl ester (1.55 g, 6.8 mmol) and 3-nitrobenzoyl chloride (1.25 g, 6.8 mmol, 1 eq.) was reacted using the procedure of step A of Example 974 to give 1.5 g (75%) of {cis-4-[(3-nitro-benzoylamino)-methyl)-cyclohexyl}-carbamic acid tert-butyl ester.

ESI MS m/e 378 M+H⁺; ¹H NMR (400 MHz, CDCl₃) δ 8.54 (t, J=2.0 Hz, 1 H), 8.33 (d, J=8.0 Hz, 1 H), 8.14 (d, J=8.0 Hz, 1 H), 7.63 (t, J=7.6 Hz, 1 H), 6.31 (brs, 1 H), 4.62 (brs, 1 H), 3.73 (brs, 1 H), 3.41 (t, J=6.4 Hz, 2 H), 1.72-1.57 (m, 7 H), 1.44 (s, 9 H), 1.32 (m, 2 H).

### Step B: Synthesis of cis-N-(4-amino-cyclohexylmethyl)-3-nitro-benzamide hydrochloride

{cis-4-[(3-Nitro-benzoylamino)-methyl]-cyclohexyl}-carbamic acid tert-butyl ester (1.4 g, 3.7 mmol) in DCM/TFA (1:1=13 mL) was stirred for 2 hr at room temperature. After removal of the volatile solvent, the residue was dissolved in DCM (10 mL), and 2M-HCl in ether (˜4 mL, 2 eq.) was added. Afer stirring for 20 min at room temperature, removal of the volatile solvent gave 1.2 g (82%) of cis-N-(4-amino-cyclohexylmethyl)-3-nitro-benzamide hydrochloride as the desired product.

ESI MS m/e 278 M+H⁺; ¹H NMR (400 MHz, DMSO-d₆) δ 8.91 (t. J=5.6 Hz, 1 H), S.65 (m, 1 H), 8.36 (d, J=2.0 Hz, 1 H). 8.29 (d, J=8.0 Hz, 1 H), 7.97 (brs, 2 H), 7.74 (t, J=8.0 Hz, 1 H), 3.25 (t, J=6.8 Hz, 2 H), 3.13 (brs, 1 H), 1.77 (m, 1 H), 1.65-1.61 (m, 4 H) 1.51 (m, 4 H).

### Step C: Synthesis of cis-N-[4-(4-dimethylamino-quinazolin-2-ylamino)-cyclohexylmethyl]-3-nitro-benzamide

A heterogeneous solution of 2-chloro-4-N,N-dimethylamino quinazoline (0.38 g, 1.45 mmol) and cis-N-(4-amino-cyclohexylmethyl)-3-nitro-benzamide hydrochloride (0.45 g, 1 eq.) in IPA (2 mL) and DIEA (0.46 mL, 2 eq.) was irradiated for 1 h 10 min. at 155° C. with a Smith microwave reactor. The reaction was quenched and purified by column chromatography (silica gel. DCM/MeOH=100:0 to 85:15). 0.57 g (87%) of the product was obtained.

ESI MS m/e 449 M+H⁺; ¹H NMR (400 MHz, CDCl₃) δ 8.91 (brs, 1 H), 8.76 (s, 1 H), 8.45 (d, J=7.6 Hz, 1 H), 8.25 (d: J=8.4 Hz, 1 H), 7.60 (d, J=8.4 Hz, 1 H), 7.60 (m, 2 H), 7.51 (brs, 1 H), 7.42 (d, J=8.4 Hz, 1 H), 7.21 (t, J=8.0 Hz, 1 H), 4.35 (brs, 1 H), 3.51 (brs, 2 H), 3.49 (s, 6 H), 1.94-1.80 (m, 5 H), 1.67-1.62 (m, 4 H).

### Step D: Synthesis of N-[(cis-4-{[4-(dimethylamino)quinazolin-2-yl]amino}cyclohexyl)methyl]-3-[(2,2-dimethylprop anoyl)amino]benzamide

A heterogenous solution of cis-N-[4-(4-dimethylamino-quinazolin-2-ylamino)-cyclohexylmethyl]-3-nitro-benzamide (0.57 g, 1.27 mmol) and 10%-Pd/C (100 mg) in EtOH (25 mL) was stirred overnight under H₂. The reaction was filtered through a pad of celite. After removal of the volatile solvent, the residue was purified from a short pad of silica gel (DCM/MeOH=100:0 to 80:20) to give 3-amino-cis-N-[4-(4-dimethylamino-quinazolin-2-ylamino)-cyclohexylmethyl]-benzamide (0.45 g, 83%, ESI MS m/e 419 M+H⁺). 3-Amino-cis-N-[4-(4-diiethylamilo-quinazolin-2-ylamino)-cyclohexylmethyl]-benzamide(30 mg, 0.07 mmol) and 2,2-dimethylpropionyl chloride (9 mmol, 0.07 mmol) was reacted in the presence of catalytic DIEA (4 drops). The product was purified from column chromatogiaplhy (silica gel, DCM/MeOH=100:0 to 90:10) to give N-[(cis-4-{[4-(dimethylamino)quinazolin-2-yl]amino}cyclohexyl) methyl]-3-[(2,2-dimethylpropanoyl)amino]benzamide (12 mg, 33%).

ESI MS m/e 503 M+H⁺; ¹H NMR (400 MHz, CDCl₃) δ 8.92 (brs, 1 H), 8.86 (s, 1 H), 8.35 (s, 1 H) 8.33 (brs, 1 H), 7.87 (d, J=8.4 Hz, 1 H), 7.61 (m, 2 H), 7.32 (t, J=8.0 Hz, 2 H), 7.22 (t, J=7.2 Hz, 1 H), 6.74 (t, J=4.8 Hz, 1 H), 4.34 (m, 1 H), 3.51 (m, 2 H), 3.48 (s, 6 H), 1.97 (m, 2 H), 1.86-1.78 (m, 3 H), 1.69-1.59 (m, 4. H), 1.44 (s, 9 H).

### Example 976

### N-[(cis-4-{[4-(Dimetlylamino)quinazolin-2-yl]amino}cyclohexyyl)methyl]-3-propionylamino)benzamide

### Step A: Synthesis of N-cis-4-{14-(dimethylamino)quinazolin-2-yl]amino}cyclohexyl)methyl]-3-(propionylamino)benzamide

Using a similar procedure as described in step D of Example 975, the title compound was obtained.

ESI MS m/e 475 M+H⁺; ¹H NMR (400 MHz, CDCl₃) δ 9.59 (brs, 1 H), 3.53 (brs, 1 H), 8.39 (brs, 1 H), 8.05 (s, 1 H), 7.87 (d, J=8.4 Hz, 1 H), 7.63 (t, J=7.6 Hz, 1 H), 7.58 (d, J=7.6 Hz, 1 H), 7.37 (m, 2 H), 7.23 (m, 1 H), 6.44 (brs, 1 H), 4.33 (m, 1 H), 3.54 (d, J=5.2 Hz, 2 H), 3.48 (s, 6 H), 2.59 (q, J=7.6 Hz, 2 H), 2.05 (m, 2 H), 1.76-1.61 (m, 7 H), 1.31 (t, J=7.6 Hz, 3 H).

### Example 977

### N-[(cis-4-{[4-(Dimethylamino)quinazolin-2-yl]amino}cyclohexyl)methyl)-3-(isobutyrylamino)benzamide

### Step A: Synthesis of N-[(cis-4-{[4-(dimethylamino)quinazolin-2-yl]amino}cyclohexyl)nethyl]-3-(isobutyrylamino)benzamide

Using a similar procedure as described in step D of Example 975, the title compound was obtained.

ESI MS m/e 489 M+H⁺; ¹H NMR (400 1MHz, CDCl₃) δ 9.59 (brs, 1 H), 8.50 (brs, 1 H), 8.40 (brs, 1 H), 8.17 (s, 1 H), 7.87 (d, J=8.4 Hz, 1 H), 7.62 (t, J 7.6 Hz, 1 H), 7.56 (d, J=7.6 Hz, 1 H), 7.35 (m, 2 H), 7.23 (m, 1 H), 6.54 (brs, 1 H), 4.3) (m, 1 H), 3.51 (d, J=5.6 Hz, 2 H), 3.48 (s, 6 H), 2.88 (m, 1 H), 2.03 (m, 2 H), 1.76-1.62 (m, 7 H), 1.32 (d, J=7.6 Hz, 6 H).

### Example 978

### N-[(cis-4-{[4-(Dimethylamino)quioazolin-2-yl]amino}cyclohexyl)methyl]-3-[(3-methylbutanoyl)amino]benzamide

### Step A: Synthesis of N-[(cis-4-{[4-(dimethylamino)quinazolin-2-yl]amino}cyclohexyl)methyll-3-[(3-methylbutanoyl)amino]benzamide

Using a similar procedure as described in step D of Example 975, the title compound was obtained.

ESI MS m/e 503 M+H⁺; ¹H MNR (400 MHz, CDCl₃) δ 9.71 (brs, 1 H), 8.60 (d, J=7.2 Hz, 1 H), 8.43 (d, J=8.4 Hz, 1 H), 8.15 (s, 1 H), 7.88 (d, J=8.4 Hz, 1 H), 7.62 (t, J=7.6 Hz, 1 H), 7.56 (d, J=7.6 Hz, 1 H), 7.35 (m, 2 H), 7.23 (m, 1 H), 6.57 (brs, 1 H), 4.32 (m, 1 H), 3.49 (s, 8 H), 2.44 (d, J=7.2 Hz, 2 H), 2.33 (m, 1 H), 2.02 (m, 2 H), 1.77-1.62 (m, 7 H), 1.07 (d, J=7.6 Hz, 6 H).

### Example 979

### 3-[(Cyclopropylcarbonyl)amino]-N-[(cis-4-{[4-(dimethylamino)quinazolin-2-yl]amino}cyclohexyl)methyl]benzamide

### Step A: Synthesis of 3-[(cyclopropylcarbonyl)amino]-N-[(cis-4-{[4-(dimethylamino)-quinazolin-2-yl]amino}cyclohexyl)methyl]benzamide

Using a similar procedure as described in step D of Example 975, the title compound was obtained.

ESI MS m/e 487 M+H⁺; ¹H NNR (400 MHz, CDCl₃) δ 10.1 (brs, 1 H), 8.60 (brs, 1 H), 8.34 (d, J=8.4 Hz, 1 H), 8.09 (s, 1 H), 7.86 (d, J=8.4 Hz, 1 H), 7.60 (t, J=7.6 Hz, 1 H), 7.54 (d, J=8.0 Hz, 1 H), 7.41 (d, J=8.4 Hz, 1 H), 7.29 (t, J=8.0 Hz, 1 H), 7.23 (m, 1 H), 6.61 (brs, 1 H), 4.28 (m, 1 H), 3.51 (d, J=6.0 Hz, 2 H), 3.48 (s, 6 H), 2.08 (m, 3 H), 1.78-1.61 (m, 7 H), 1.09 (m, 2 H), 0.87 (m, 2 H).

### Example 980

### 3-[(Cyclobutylcarbonyl)amino]-N-[(cis-4-{[4-(dimethylamino)quinazolin-2-yl]amino}cyclohexyl)methyl]benzamide

### Step A: Synthesis of 3-[(cyclobutylcarbonyl)aminol-N-[(cis-4-{[4-(dimethylamino)quinazolin-2-yl]amino}cyclohexyl)methyl]benzamide

Using a similar procedure as described in step D of Example 975, the title compound was obtained.

ESI MS m/e 501 M+H⁺; ¹H NMR (400 MHz, CDCl₃) δ 9.45 (brs, 1 H), 8.68 (brs, 1 H), 8.41 (d, J=7.2 Hz, 1 H), 8.13 (s, 1 H), 7.87 (d, J=8.4 Hz, 1 H), 7.63 (t, J=7.6 Hz, 1 H), 7.56 (d, J=7.6 Hz, 1 H), 7.40 (d, J=7.6 Hz, 1 H), 7.32 (t, J=7.6 Hz, 1 H), 7.23 (m, 1 H), 6.50 (brs, 1 H), 4.32 (m, 1 H), 3.51 (d, J=5.6 Hz, 2 H), 3.49 (s, 6 H), 2.48 (m, 2 H), 2.31 (m, 2 H), 2.06-1.59 (m, 12 H).

### Example 981

### 3-1(Cyclopentylcabonyl)amino]-N-[(cis-4-{[4-(dmethylamino)quinazolin-2-yl]amino}cyclohexyl)methyl]benzamide

### Step A: Synthesis of 3-[(cyclopentylcarbonyl)amino]-N-[(cis-4-{[4-(dimethylamino)-quinazolin-2-yl]amino}cyclohexyl)methyl]benzamide

Using a similar procedure as described in step D of Example 975, the title compound was obtained.

ESI MS m/e 515 M+H⁺; ¹H NMR (400 MHz, CDCl₃) δ 9.60 (brs, 1 H), 8.56 (brs, 1 H), 8.40 (d, J=5.6 Hz, 1 H), 8.17 (s, 1 H), 7.87 (d, J=8.4 Hz, 1 H), 7.61 (t, J=7.6 Hz, 1 H), 7.56 (d, J=7.6 Hz, 1 H), 7.37 (d, J=7.6 Hz, 1 H), 7.33 (t, J=7.6 Hz, 1 H), 7.23 (m, 1 H), 6.50 (brs, 1 H), 4.32 (m, 1 H) 3.52 (d, J=5.2 Hz, 2 H), 3.48 (s, 6 H), 3.05 (m, 1 H), 2.06-1.60 (m, 17 H).

### Example 982

### 3-[(Cyclohexylcarbonyl)amino]-N-(cis-4-{[4-(dimethylamino)quinazolin-2-yl]amino}cyclohexyl)methyl]benzamide

### Step A: Synthesis of 3-[(cyclohexlcarbonyl)amino]-N-[(cis-4-{[4-(dimethylamino)quinazolin-2-yl]amino}cyclohexyl)nethyl]benzamide

Using a similar procedure as described in step D of Example 975, the title compound was obtained.

ESI MS m/e 529 M+H⁺; ¹H NMR (400 MHz, CDCl₃) δ 9.53 (brs, 1 H), 8.61 (brs, 1 H), 8.40 (d, J=6.8Hz, 1 H), 8.20 (s, 1 H), 7.87 (d, J=7.6 Hz, 1 H), 7.60 (t, J=7.6 Hz, 1 H), 7.56 (d, J=7.6 Hz, 1 H), 7.34 (m, 2 H), 7.23 (m, 1 H), 6.49 (brs, 1 H), 4.33 (m, 1 H), 3.53 (d, J=4.0 Hz, 2 H), 3.49 (s, 6 H), 2.59 (m, 1 H), 2.06-1.60 (m, 19 H).

### Example 983

### cis-4-{[4-(Dimethylamino)quinazolin-2-yl]amino}-N-{3-[(2,2-dimethylpropanoyl)amino]-benzyl}cyclohexanecarboxamide

### Step A: Synthesis of cis-[-4-(3-nitrogencyclocarbamoyl)-cyclobenzyl]-carbamic acid tert-butyl ester

cis-4-(tert-Butoxycarbonylamino)-cyclohexanecarboxylic acid (2.0 g, 8/2 mmol) and 3-nitrobenzyl amine hydrochloride (1.54 g, 8.2 mmol, 1 eq) in DCM (30 mL was reacted in the presence of HATU (3.5 g, 9.02 mmol, 1.1 eq.) and Et₃N (˜4 mL). The reaction was diluted with DCM, washed with 1N-HCl and water, dried over MgSO₄, and concentrated. From column chromatography (silica gel, DCM/MeOH =100:0 to 95 to 5), 2.7 g (90%) of cis-[-4-(3-nitrobenzylcarbamoyl)-cyclohexyl]-carbamic acid tert-butyl ester was isolated.

ESI MS m/e 378 M+H⁺; ¹H NMR (400 MHz, CDCl₃) δ 8.11 (brs, 1 H), 8.09 (s, 1 H), 7.60 (d, J=8.0 Hz, 1 H), 7.48 (t, J=7.6 Hz, 1 H), 6.17 (brs, 1 H), 4.72 (bis, 1 H), 4.53 (d, J=6.0 Hz, 2 H), 3.74 (brs, 1 H), 2.27 (m, 1 H), 1.80-1.71 (m, 6 H), 1.65-1.59 (m, 2 H), 1.45 (s, 9 H).

### Step B: Synthesis of cis-4-amino-cyclohexanecarboxylic acid 3-nitro-benzamide hydrochloride

cis-[4-(3-Nitrobenzylcarbamoyl)-cyclohexyl]-carbamic acid tert-butyl ester (2.5 g, 6.6 mmol) was reacted in TFA/DCM. (1:2=23 mL) for 2 hr at room temperature. After removal of the solvents, the residue was dissolved in DCM (15 mL), and added 2M-HCl in ethyl ether (2 eq.). After stirring for 20 min at room temperature, the volatile solvent was removed to give cis-4-amino-cyclohexanecarboxylic acid 3-nitro-benzamide hydrochloride (2.0 g, 95%) as a yellowish white solid.

ESI MS m/e 278 M+H⁺; ¹H NMR (400 MHz, DMSO-d₆) δ 8.53 (t, J=6.0 Hz, 1 H), 8.07 (d, J=7.6 Hz, 1 H), 8.06 (s, 1 H), 7.84 (brs, 2 H), 7.68 (d, J=7.6 Hz, 1 H), 7.59 (t, J=7.6 Hz, 1 H), 4.37 (d, J=6.4 Hz, 2 H), 3.13 (m, 1 H), 2.40 (m, 1 H), 1.89 (m, 2 H), 1.68 (m, 4 H), 1.57 (m, 2 H).

### Step C: Synthesis of cis-4-(4-dimethylamino-quinazolin-2-ylamino)-cyclohexanecarboxylic acid 3-nitro-benzamide

A solution of 2-chloro-4-N,N-dimethylamino quinazoline (0.35 g, 1.7 mmol) and cis-4-amino-cyclohexanecarboxylic acid 3-nitro-benzamide hydrochloride (0.5 g, 1 eq.) in IPA (2.5 mL) and DIEA (0.7 mL) Harrs reacted for 1 h 10 min. at 155° C. in a Smith synthesizer. The reaction was quenicihed and purified by column chromatography (silica gel, DCM/MeOH=100:0 to 85:15). 0.56 g (75%) of cis-4-(4-dimethylamino-quinazolin-2-ylamino)-cyclohexanecarboxylic acid 3-nitro-benzamide was isolated.

ESI MS m/e 449 M+H⁺; ¹H NMR (400 MHz, CDCl₃) δ 9.12 (brs, 1 H), 8.24 (brs, 1 H), 8.15 (s, 1 H), 8.03 (d, J=8.0 Hz, 1 H), 7.88 (d, J=8.0 Hz, 1 H), 7.69 (d, J=8.0 Hz, 1 H), 7.62 (t, J=8.0 Hz, 1 H), 7.44 (m, 2 H), 7.24 (t, J=7.6 Hz, 1 H), 4.54 (d, J=6.4 Hz, 2 H), 4.48 (m, 1H)3.50 (s, 6 H), 2.43 (tt, J=12.4, 4.0 Hz, 1 H), 2.16 (m, 2 H), 1.90 (m, 4 H), 1.63 (m, 2 H).

### Step D: Synthesis of cis-4(4-dimethylamino-quinazolin-2-ylamino)-cyclohexanecarboxylic acid 3-aminobenzyl amide

A heterogenous solution of cis-4-(4-dimethylamino-quinazolin-2-ylamino)-cyclohexanecarboxylic acid 3-nitro-benzamide (0.55 g, 1.2 mmol) and 10% Pd/C (100 mg) in EtOH (15 mL) was stirred overnight under H₂ atmosphere at room temperatutre. The reaction was filtered through a pad of celite. After removal of the volatile solvent, the residue was purified from a short pad of silica gel (DCM/MeOH=100:0 to 80:20) to give 0.46 g (91%) of cis-4(4-dimethylamino-quinazolin-2-ylamino)-cyclohexane carboxylic acid 3-aminobenzyl amide.

ESI MS m/e 419 M+H⁺; ¹H NMR (400 MHz, CDCl₃) δ 9.00 (brs, 1 H), 7.86 (d, J=8.4 Hz, 1 H), 7.59 (t, J=8.0 Hz, 1 H), 7.45 (d, J=8.4 Hz, 1 H), 7.37 (brs, 1 H), 7.22 (t, J=7.6 Hz, 1 H), 7.01 (t, J=7.6 Hz, 1 H), 6.73 (s, 1 H), 6.66 (d, J=7.6 Hz, 1 H), 6.49 (d, J=7.6 Hz, 1 H), 4.39 (m, 1 H), 4.35 (d, J=6.0 Hz, 2 H), 3.80 (brs, 2 H), 3.47 (s, 6 H), 2.36 (m, 1 H), 2.05 (m, 4 H), 1.88 (m, 4 H), 1.63 (m, 2 H).

### Step E: Synthesis of cis-4-{[4-(dimethylaminio)quinazolin-2-yl]amino}-N-{3-[(2,2-dimethyl-propanoyl)amino]benzyl}cyclohexanecarboxamide

Using a similar procedure as described in step D of Example 975, the title compound was obtained.

ESI MS m/e 503 M+H⁺; ¹H NMR (400 MHz, CDCl₃) δ 9.05 (brs, 1 H), 8.13 (brs, 1 H), 7.89 (brs, 1 H), 7.87 (d, J=8.0 Hz, 1 H), 7.62 (t, J=7.6 Hz, 1 H), 7.49 (s, 1 H), 7.38 (d J=8.4 Hz, 1 H), 7.22 (t, J=8.0 Hz, 2 H), 7.01 (brs, 1 H), 7.00 (d, J=7.2 Hz, 1 H), 4.43 (d, J=5.6 Hz, 2 H), 4.39 (m, 1 H), 3.48 (s, 6 H), 2.37 (tt, J=120 , 3.6 Hz, 1 H), 2.07 (m, 2 H), 1.97 (m, 4 H), 1.63 (m, 2 H), 1.36(s,9 H).

### Example 984

### cis-4-{[4-(Dmethylamino)quinazolin-2-yl]amino}-N-[3-(propionylamino)benzyl]cyclobexanecarboxamide

### Step A: Synthesis of cis-4-{[4-(dimethylamino)quinazolin-2-yl]amino}-N-(3-(propionylamino)benzyl]cyclohexanecarboxamide

Using a similar procedure as described in step D of Example 975, the title compound was obtained.

ESI MS m/e 475 M+H⁺; ¹H NMR (400 MHz, CDCl₃) δ 8.96 (m, 2 H), 8.04 (d, J=8.4 Hz, 1 H), 7.88 (d, J=8.0 Hz, 1 H), 7.64 (t, J=7.6 Hz, 1 H), 7.41 (d, J=8.4 Hz, 1 H), 7.37 (s, 1 H), 7.27-7.18 (m, 2 H), 6.91 (d, J=7.6 Hz, 1 H), 6.70 (brs, 1 H), 4.45 (d, J=5.6 Hz, 2 H), 4.39 (m, 1 H), 3.50 (s, 6 H), 2.53 (q, J=7.6 Hz, 2 H), 2.37 (m, 1 H), 2.04-.94 (m, 6 H), 1.66 (m, 2 H), 1.25 (t, J=7.6 Hz, 3 H).

### Example 985

### cis-4-{[4-Dimethylamino)quinazolin-2-yl]amino}-N-[3-isobutyrylamino)benzyl]cyclohexanecarboxamide

### Step A: Synthesis of cis-4-{[4-(dimethylamino)quinazolin-2-yl]amino}-N-[3-(isobutyrlamino) benzyl]cyclohexanecarboxamide

Using a similar procedure as described in step D of Example 975, the title compound was obtained.

ESI MS m/e 489 M+H⁺; ¹H NMR (400 MHz, CDCl₃) δ 8.91 (brs, 2 H), 8.04 (d, J=7.2 Hz, 1 H), 7.88 (d, J=7.6 Hz, 1 H), 7.64 (t, J=7.6 Hz, 1 H), 7.42 (s, 1 H), 7.40 (d, J=8.0 Hz 1 H), 7.27-7.18 (m, 2 H), 6.92 (d, J=8.0 Hz, 1 H), 6.70 (brs, 1 H), 4.44 (d, J=5.6 Hz, 2 H), 4.39 (m, 1 H), 3.49 (s, 6H), 2.80 (m, 1 H), 2.37 (m, 1 H), 2.05-1.94 (m,6 H), 1.66 (m, 1 H), 1.26 (d,J=6.4 Hz,6H).

### Example 986

### cis-N-{3-[(Cyclopropylcarbonyl)amino]benzyl}-4-{[4-(dimethylamino)quinazolin-2-yl]amino}cyclohexanecarboxamide

### Step A: Synthesis of cis-N-{3-[(cyclopropylcarbonyl)amino]benzyl}-4-{[4-(dimethylamino)-quinazolin-2-yl]amino}cyclohexanecarboxamide

Using a similar procedure as described in step D of Example 975, the title compound was obtained.

ESI MS mn/e 487 M+H⁺; ¹H NMR (400 MHz, CDCl₃) δ 9.24 (brs, 1 H), 9.00 (brs, 1 H), 7.99 (d, J=8.0 Hz, 1 H), 7.99 (d, J=8.0 Hz, 1 H), 7.62 (t, J=7.6 Hz, 1 H), 7.40 (d, J=8.0 Hz, 1 H), 7.36 (s, 1 H), 7.27-7.15 (m, 2 H), 6.90 (d, J=6.8 Hz, 1 H), 6.81 (brs, 1 H), 4.45 (d, J=5.6 Hz, 2 H), 4.40 (m, 1 H), 3.49 (s, 6 H), 2.37 (m, 1 H), 2.08-1.94 (m, 7 H), 1.66 (m, 2 H), 1.03 (m, 2 H), 0.80 (m, 2 H).

### Example 987

### cis-N-{3-[(Cyclopentylcarbonyl)amino]benzyl}-1-({[4-(dimethylamino)quinazolin-2-yl]amino}cyclohexanecarboxamide

### Step A: Synthesis of cis-N-{3-[(cyclopentylcarbonyl)amino]benzyl}-4-{[4-(dimethylamino)-quinazolin-2-yl]amino}cyclohexanecarboxamide

Using a similar procedure as described in step D of Example 975, the title compound was obtained.

ESI MS m/e 515 M+H⁺; ¹H NMR (400 1MHz, CDCl₃) δ 8.88 (brs, 1 H), 8.87 (brs, 1 H), 8.02 (d, J=7.2 Hz, 1 H), 7.88 (d, J=8.4 Hz, 1 H), 7.63 (t, J=7.6 Hz, 1 H), 7.40 (s, 1 H), 7.39 (d, J=8.0 Hz, 1 H), 7.27-7.17 (m, 2 H), 6.92 (d, J=7.6 Hz, 1 H), 6.74 (brs, 1 H), 4.44 (d, J=6.0 Hz, 2 H), 4.40 (m, 1 H), 3.49 (s, 6 H), 2.95 (m, 1 H), 2.37 (m, 1 H), 2.04-1.65 (m, 16 H).

### Example 988

### cis-N-{3-[(Cyclohexylcarbonyl)amino]benzyl}-4-{[4-(dimethylamino)quinazolin-2-yl]amino}cyclohexanecarboxamide

### Step A: Synthesis of cis-N-{3-[(cyclobexylcarbonyl)amino]benzyl}-4-{[4-(dimethylamino)-quinazolin-2-yl]amino}cyclohexanecarboxamide

Using a similar procedure as descnbed in step D of Example 975, the title compound was obtained.

ESI MS mte 515 M+H⁺; ¹H NMR (400 MHz, CDCl₃) δ 9.06 (brs, 1 H), 8.66 (brs, 1 H), 8.02 (d, J=6.8 Hz, 1 H), 7.88 (d, J=8.0 Hz, 1 H), 7.62 (t, J=7.6 Hz, 1 H), 7.41 (d, J=8.4 Hz, 1 H), 7.40 (s, 1 H), 7.26-7.1 8 (m, 2 H), 6.93 (d, J=8.0 Hz, 1 H), 6.81 (brs, 1 H), 4.45 (d, J=5.6 Hz, 2 H), 4.41 (brs, 1 H), 3.49 (s, 6 H), 2.4S (m, 1 H), 2.37 (m, 1 H), 2.09-1.25 (m, 18 H).

### Example 989

### 3-Chloro-N-(cis-4-{[4-(dimethylamino)-6,7-difluoroquinazolin-2-yl]amino}cyclohexyl)-benzamide

### Step A: Synthesis of [cis-4-(4-dimethylamino-6,7-difluoro-quinazolin-2-yamino)-cyclohexyl) carbamic acid tert-butyl ester

A suspended solution of 2-chloro-6,7-difluoro-4-dimethylamino quinazolinie (0.52 g: 2.1 mmol) and cis-(4-amino-cyclohexyl)-carbamic acid tert-butyl ester (0.45 g, 1 eq.) in IPA (2.5 mL) and DIEA (1 mL. ˜2 eq.) was reacted for 2 h 30 min at 155° C. in a Smith microwave synthesizer. The reaction was quenched and purified by column chromatography (DCM:MeOH=100:0 to 90:10) to give 0.28 g (33%) of [cis-4-(4-dimethylamino-6,7-difluoro-quinazolin-2-yamino)-cyclohexyl]carbamic acid tert-butyl ester.

ESI MS m/e 422 M+H⁺; ¹H NMR (400 MHz, DMSO-d₆) δ 8.10 (brs, 1 H), 7.40 (brs, 1 H), 6.80 (brs, 1 H), 4.02 (q, J=7.0 Hz, 1 H), 3.82 (brs, 1 H), 3.30 (s, 6 H), 1.65-1.50 (m, 8 H), 1.30 (s, 9 H).

### Step B: Synthesis of cis-4-(4-dimethylamino-6,7-difluoro-quinazolin-2-yamino)-4-aminocyclohexane trifluoroacetate

A solution of [cis-4-(4-dimethylamino-6,7-difluoro-quinazolin-2-yamino)-cyclohexyl]carbamic acid tert-butyl ester (0.28 g, 0.66 mmol) in TFA/DCM (1:2=16 mL) was stirred at room temperature for 1.5 hr. After removal of the volatile solvent, the crude product (0.27 g, 95%) was directly used to next reaction without a further purification.

ESI MS m/e 322 M+H⁺.

### Step C: Synthesis of 3-chloro-N-(cis-4-{[4-(dimethylamino)-6,7-difluoroquinazolin-2-yl]amino}cyclobexyl)benzanide

cis-4-(4-Dimethylamino-6,7-difluoro-quinazolin-2-yamino)-4-amino cyclohexane trifluoroacetate (25 mg, 0.06 mmol) and 3-chlorobenzoyl chloride (10 mg, 0.06 mmol) was stirred overnight at room temperature in the presence of a catalytic amount of DIEA (3 drops). The compounds vere purified from prep-HPLC to give 3-chloro-N-(cis-4-{[4-(drimethylamino)-6,7-difluoroquinazolin-2-yl]amino}cyclohexyl)benzamide (9 mg, 27%).

ESI MS m/e 460 M+H⁺; ¹H NMR (400 MHz, DMSO-d₆) δ 12.06 (brs, 1 H), 8.29 (brs, 1 H), 8.23 (m, 1 H), 8.04 (brs, 1 H), 7.83 (s, 1 H), 7.74 (d, J=8.0 Hz, 1 H), 7.54 (d, J=8.0 Hz, 1 H), 7.20 (brs, 1 H), 7.44 (t, J=8.0 Hz, 1 H), 3.98 (brs, 1 H), 3.36 (brs, 1 H), 3.36 (s, 6 H), 1.82 (brs, 2 H), 1.68 (brs, 6 H).

### Example 990

### 3,4-Dichloro-N-(cis-4-{[4-(dimethylamino)-6,7-difluoroquinazolin-2-yl]amino}cyclohexyl)-benzamide

### Step A: Synthesis of 3,4-dicloro-N-(cis-4-{[4-(dmethylamino)-6,7-difluoroquinazolin-2-yl]-amino}cyclobexyl)benzamide

Using a similar procedure as described in step C of Example 989, the title compound was obtained.

ESI MS m/e 496 M+H⁺; ¹H NMR (400 MHz, DMSO-d₆) δ 12.6 (brs, 1 H), 8.36 (brs, 1 H), 8.28 (brs, 1 H), 8.20 (m, 1 H), 8.03 (d, J=2.0 Hz, 1 H), 7.77 (dd, J=8.0, 2.0 Hz, 1 H), 7.69 (d, J=8.0 Hz, 1 H), 7.45 (brs, 1 H), 3.98 (brs, 1 H), 3.83 (brs, 1 H), 3.41 (s, 6 H), 1.83 (brs, 2 H), 1.68 (brs, 6 H).

### Example 991

### N-(cis-4-{[4-(Dimethylamino)-6,7-difluoroquinazolin-2-yl]amino}cyclohexyl)-3,5-dimethoxybenzamide trifluoroacetate

### Step A: Synthesis of N-(cis-4-{[4-(dimethylamino)-6,7-difluoroquinazolin-2-yl]amino}-cyclohexyl)-3,5-dimethoxybenzamide trifluoroacetate

Using a similar procedure as described in step C of Example 989, the title compound was obtained.

ESI MS m/e 486 M+H^{+;} ¹H NMR (400 MHz, DMSO-d₆) δ 12.1 (brs, 1 H), 8.20 (m, 1 H), 8.09 (brs, 2H), 7.50 (m, 1 H), 6.92 (d, J=2.0 Hz, 2 H), 6.58 (t, J=2.0 Hz, 1 H), 4.00 (brs, 1 H), 3.80 (brs, 1 H), 3.72 (s, 6 H), 3.37 (s, 6 H), 1.82 (brs, 2 H), 1.67 (brs, 6 H).

### Examples 992-1008

Compounds 992 to 1008 were prepared in a similar manner as described in Example 890 using the appropriate benzylamine and the carboxylic acid intermediate from Step E.

### Examples 1009-1014

Compounds 1009 to 1014 were prepared in a similar manner as described in Example 893 using the appropriate isocyanate (i.e., Compound 1009 to 1013) or thioisocyanate (i.e., Compound 1014) and the amine intermediate from Step D.

### Examples 1015-1029

Compounds 1015 to 1029 were prepared in a similar manner as described in Example 894 using the appropriate isocyanate and the amine intermediate from Step E.

### Examples 1030-1043

Compounds 1030 to 1043 were prepared in a similar manner as described in Example 896 using the appropriate phenol and the nicotinamide intermediate from Step A.

### Examples 1044-1049

Compounds 1044 to 1049 were prepared in a similar manner as described in Example 902 using the appropriate benzaldehyde and the amine intermediate from Step C.

### Examples 1050-1072

Compounds 1050 to 1072 were prepared in a similar manner as described in Example 903 using the appropriate phenol and the nicotinamide intermediate from Step A.

### Examples 1073 and 1074

Compounds 1073 and 1074 inwere prepared in a similar manner as described in Example 905 using the appropriate phenol and the nicotinamide intermediate from Step A.

### Examples 1075-1034

Compounds 1075 to 1084 were prepared in a similar manner as described in Example 907 using the appropriate phenoxyacetic acid and the amine intermediate from the Example in 895 Step B.

### Examples 1085-1091

Compounds 1085 to 1091 were prepared in a similar manner as described in Example 912 using the appropriate aniline and the bromoacetamide.

### Examples 1092-1104

Compounds 1092 to 1104 were prepared in a similar manner as described in Example 913 using the appropriate carboxylic acid and the amine intermediate from Step C.

### Examples 1105-1115

Compounds 1105 to 1115 were prepared in a simnilar manner as described in Example 914 using the appropriate carboxylic acid and the amine intermediate from the Example in 895 Step B.

### Examples 1116-1119

Compounds 1116 to 1119 wvere prepared in a similar manner as described in Example 915 using the appropriate benzylamine and the carboxylic acid intermediate from Step D.

### Examples 1120-1130

Compounds 1120 to 1130 were prepared in a similar manner as described in Example 917 using the appropriate acid chloride and the amine intermediate from Step D.

### Example 1131

Compound 1131 was prepared in a similar manner as described in Example 1 using 3,5-dichlorobenzaldehyde.

### Examples 1132 and 1133

Compounds 1132 and 1133 were prepared in a similar manner as described in Example 919 using the appropriate acid chloride and the amine intermediate from Step A.

### Example 1134

Compound 1134 was prepared in a similar manner as described in Example 920 using the appropriate benzaldehyde and the amine intermediate from Example 919 Step A.

### Examples 1135-1195

Compounds 1135 to 1195 were prepared in a similar manner as described in Example 921 using the appropriate arylamine and the carboxylic acid intermediate from Step C.

### Examples 1196-1199

Compounds 1 196 to 1199 were prepared in a similar manner as described in Example 951 using the appropriate arylamine and the acid chloride intermediate from Step A.

### Examples 1200-1204

Compounds 1200 to 1204 were prepared in a similar manner as described in Example 974 using the appropriate acid chloride and aniline intermediate from Step C.

### Examples 1205-1211

Compounds 1205 to 1211 were prepared in a similar manner as described in Example 989 using the appropriate acid chloride and amine intermediate from Step B.

| Ex. No. | compound name | MS | class |
|---|---|---|---|
| 992 | cis-N-benzyl-4-{[4-(dimethylamino)-6,7-difluoroquinazolin-2- | 440.5 | 2 |
| | yl]amino}cyclohexanecarboxamide | | |
| 993 | cis-N-(3,5-dimethoxybenzyl)-4-{[4-(dimethylamino)-6,7- | 500.4 | 2 |
| | difluoroquinazolin-2-yl]amino}cyclohexanecarboxamide | | |
| 994 | cis-4-{[4-(dimethylamino)-6,7-difluoroquinazolin-2-yl]amino}-N- | 470.4 | 1 |
| | (4-methoxybenzyl)cyclohexanecarboxamide | | |
| 995 | cis-4-{[4-(dimethylamino)-6,7-difluoroquinazolin-2-yl]amino}-N- | 470.3 | 2 |
| | (3-methoxybenzyl)cyclohexanecarboxamide | | |
| 996 | cis-N-[(6-chloropyridin-3-yl)methyl]-4-{[4-(dimethylamino)-6,7- | 475.3 | 2 |
| | difluoroquinazolin-2-yl]amino}cyclohexanecarboxamide | | |
| 997 | cis-N-(2,4-difluorobenzyl)-4-{[4-(dimethylamino)-6,7- | 476.3 | 3 |
| | difluoroquinazolin-2-yl]amino}cyclohexanecarboxamide | | |
| 998 | cis-4-{[4-(dimethylamino)-6,7-difluoroquinazolin-2-yl]amino}-N- | 508.5 | 1 |
| | [3-(trifluoromethyl)benzyl]cyclohexanecarboxamide | | |
| 999 | cis-4-{[4-(dimethylamino)-6,7-difluoroquinazolin-2-yl]amino}-N- | 508.4 | 2 |
| | [4-(trifluoromethyl)benzyl]cyclohexanecarboxamide | | |
| 1000 | cis-N-(2,4-dichlorobenzyl)-4-{[4-(dimethylamino)-6,7- | 508 | 1 |
| | difluoroquinazolin-2-yl]amino}cyclohexanecarboxamide | | |
| 1001 | cis-N-(3,5-dichlorobenzyl)-4-{[4-(dimethylamino)-6,7- | 508 | 1 |
| | difluoroquinazolin-2-yl]amino}cyclohexanecarboxamide | | |
| 1002 | cis-N-(4-bromobenzyl)-4-{[4-(dimethylamino)-6,7- | 518.2 | 1 |
| | difluoroquinazolin-2-yl]amino}cyclohexanecarboxamide | | |
| 1003 | cis-N-(2-bromobenzyl)-4-{[4-(dimethylamino)-6,7- | 518.2 | 1 |
| | difluoroquinazolin-2-yl]amino}cyclohexanecarboxamide | | |
| 1004 | cis-4-{[4-(dimethylamino)-6,7-difluoroquinazolin-2-yl]amino}-N- | 524.6 | 1 |
| | [4-(trifluoromethoxy)benzyl]cyclohexanecarboxamide | | |
| 1005 | cis-N-[3,5-bis(trifluoromethyl)benzyl]-4-{[4-(dimethylamino)- | 576.2 | 3 |
| | 6,7-difluoroquinazolin-2-yl]amino}cyclohexanecarboxamide | | |
| 1006 | cis-4-{[4-(dimethylamino)-6,7-difluoroquinazolin-2-yl]amino}-N- | 566.2 | 2 |
| | (3-iodobenzyl)cyclohexanecarboxamide | | |
| 1007 | cis-4-{[4-(dimethylamino)-6,7-difluoroquinazolin-2-yl]amino}-N- | 468.4 | 1 |
| | [(1S)-1-(4-methylphenyl)ethyl]cyclohexanecarboxamide | | |
| 1008 | cis-N-[1-(4-bromophenyl)ethyl]-4-{[4-(dimethylamino)-6,7- | 532.2 | 2 |
| | difluoroquinazolin-2-yl]amino}cyclohexanecarboxamide | | |
| 1009 | N-(cis-4-{[4-(dimethylamino)quinazolin-2-yl]amino}cyclohexyl)- | 489.4 | 1 |
| | N′-[3-(trifluoromethoxy)phenyl]urea | | |
| 1010 | N-(3,4-dimethoxyphenyl)-N′-(cis-4-{[4- | 465.2 | 1 |
| | (dimethylamino)quinazolin-2-yl]amino}cyclohexyl)urea | | |
| 1011 | N-(2-chlorophenyl)-N′-(cis-4-{[4-(dimethylamino)quinazolin-2- | 439.4 | 2 |
| | yl]amino}cyclohexyl)urea | | |
| 1012 | N-(2,6-dichlorophenyl)-N′-(cis-4-{[4-(dimethylamino)quinazolin- | 473.4 | 3 |
| | 2-yl]amino}cyclohexyl)urea | | |
| 1013 | N-(2,3-dichlorophenyl)-N′-(cis-4-{[4-(dimethylamino)quinazolin- | 473.4 | 3 |
| | 2-yl]amino}cyclohexyl)urea | | |
| 1014 | N-(2-bromophenyl)-N′-(cis-4-{[4-(dimethylamino)quinazolin-2- | 499.4 | 2 |
| | yl]amino}cyclohexyl)thiourea | | |
| 1015 | N-[(cis-4-{[4-(dimethylamino)quinazolin-2- | 449.4 | 2 |
| | yl]amino}cyclohexyl)methyl]-N′-(2-methoxyphenyl)urea | | |
| 1016 | N-[(cis-4-{[4-(dimethylamino)quinazolin-2- | 463.4 | 2 |
| | yl]amino}cyclohexyl)methyl]-N′-(2-ethoxyphenyl)urea | | |
| 1017 | N-[(cis-4-{[4-(dimethylamino)quinazolin-2- | 449.4 | 2 |
| | yl]amino}cyclohexyl)methyl]-N′-(3-methoxyphenyl)urea | | |
| 1018 | N-(3,4-dimethoxyphenyl)-N′-[(cis-4-{[4- | 479.4 | 3 |
| | (dimethylamino)quinazolin-2-yl]amino}cyclohexyl)methyl]urea | | |
| 1019 | N-(2,4-dimethoxyphenyl)-N′-[(cis-4-{[4- | 479.4 | 2 |
| | (dimethylamino)quinazolin-2-yl]amino}cyclohexyl)methyl]urea | | |
| 1020 | N-(2,5-dimethoxyphenyl)-N′-[(cis-4-{[4- | 479.4 | 2 |
| | (dimethylamino)quinazolin-2-yl]amino}cyclohexyl)methyl]urea | | |
| 1021 | N-(2-chlorophenyl)-N′-[(cis-4-{[4-(dimethylamino)quinazolin-2- | 453.4 | 2 |
| | yl]amino}cyclohexyl)methyl]urea | | |
| 1022 | N-(3-chlorophenyl)-N′-[(cis-4-{[4-(dimethylamino)quinazolin-2- | 453.4 | 2 |
| | yl]amino}cyclohexyl)methyl]urea | | |
| 1023 | N-(4-chlorophenyl)-N′-[(cis-4-{[4-(dimethylamino)quinazolin-2- | 453.4 | 2 |
| | yl]amino}cyclohexyl)methyl]urea | | |
| 1024 | N-(3,5-dichlorophenyl)-N′-[(cis-4-{[4- | 487.4 | 1 |
| | (dimethylamino)quinazolin-2-yl]amino}cyclohexyl)methyl]urea | | |
| 1025 | N-(2,6-dichlorophenyl)-N′-[(cis-4-{[4- | 487.4 | 1 |
| | (dimethylamino)quinazolin-2-yl]amino}cyclohexyl)methyl]urea | | |
| 1026 | N-(3,4-dichlorophenyl)-N′-[(cis-4-{[4- | 487.4 | 2 |
| | (dimethylamino)quinazolin-2-yl]amino}cyclohexyl)methyl]urea | | |
| 1027 | N-(2,5-dichlorophenyl)-N′-[(cis-4-{[4- | 487.4 | 2 |
| | (dimethylamino)quinazolin-2-yl]amino}cyclohexyl)methyl]urea | | |
| 1028 | N-(2,3-dichlorophenyl)-N′-[(cis-4-{[4- | 487.4 | 1 |
| | (dimethylamino)quinazolin-2-yl]amino}cyclohexyl)methyl]urea | | |
| 1029 | N-(2,4-dichlorophenyl)-N′-[(cis-4-{[4- | 487.4 | 2 |
| | (dimethylamino)quinazolin-2-yl]amino}cyclohexyl)methyl]urea | | |
| 1030 | N-(cis-4-{[4-(dimethylamino)quinazolin-2-yl]amino}cyclohexyl)- | 501.30 | 1 |
| | 2-(2-fluorophenoxy)nicotinamide | | |
| 1031 | 2-(2-chlorophenoxy)-N-(cis-4-{[4-(dimethylamino)quinazolin-2- | 517.40 | 1 |
| | yl]amino}cyclohexyl)nicotinamide | | |
| 1032 | 2-(2-bromophenoxy)-N-(cis-4-{[4-(dimethylamino)quinazolin-2- | 561.30 | 1 |
| | yl]amino}cyclohexyl)nicotinamide | | |
| 1033 | N-(cis-4-{[4-(dimethylamino)quinazolin-2-yl]amino}cyclohexyl)- | 567.40 | 1 |
| | 2-[2-(trifluoromethoxy)phenoxy]nicotinamide | | |
| 1034 | N-(cis-4-{[4-(dimethylamino)quinazolin-2-yl]amino}cyclohexyl)- | 501.50 | 1 |
| | 2-(3-fluorophenoxy)nicotinamide | | |
| 1035 | N-(cis-4-{[4-(dimethylamino)quinazolin-2-yl]amino}cyclohexyl)- | 513.40 | 1 |
| | 2-(3-methoxyphenoxy)nicotinamide | | |
| 1036 | N-(cis-4-{[4-(dimethylamino)quinazolin-2-yl]amino}cyclohexyl)- | 567.50 | 1 |
| | 2-[3-(trifluoromethoxy)phenoxy]nicotinamide | | |
| 1037 | N-(cis-4-{[4-(dimethylamino)quinazolin-2-yl]amino}cyclohexyl)- | 567.40 | 1 |
| | 2-[4-(trifluoromethoxy)phenoxy]nicotinamide | | |
| 1038 | 2-(3,4-difluorophenoxy)-N-(cis-4-{[4-(dimethylamino)quinazolin- | 519.60 | 1 |
| | 2-yl]amino}cyclohexyl)nicotinamide | | |
| 1039 | 2-(3,5-dimethoxyphenoxy)-N-(cis-4-{[4- | 543.20 | 1 |
| | (dimethylamino)quinazolin-2-yl]amino}cyclohexyl)nicotinamide | | |
| 1040 | 2-(2,3-dimethoxyphenoxy)-N-(cis-4-{[4- | 543.20 | 1 |
| | (dimethylamino)quinazolin-2-yl]-amino}cyclohexyl)nicotinamide | | |
| 1041 | N-(cis-4-{[4-(dimethylamino)quinazolin-2-yl]amino}cyclohexyl)- | 573.50 | 1 |
| | 2-(3,4,5-trimethoxyphenoxy)nicotinamide | | |
| 1042 | 2-(4-chloro-3-fluorophenoxy)-N-(cis-4-{[4- | 535.10 | 1 |
| | (dimethylamino)quinazolin-2-yl]amino}cyclohexyl)nicotinamide | | |
| 1043 | 2-(3-chloro-4-fluorophenoxy)-N-(cis-4-{[4- | 535.40 | 1 |
| | (dimethylamino)quinazolin-2-yl]amino}cyclohexyl)nicotinamide | | |
| 1044 | N2-{(1S,3R)-3-[(2,4-dimethoxybenzyl)amino]cyclopentyl}- | 422 | 3 |
| | N4,N4-dimethylquinazoline-2,4-diamine | | |
| 1045 | N4,N4-dimethyl-N2-((1S,3R)-3-{[3-(trifluoromethyl)benzyl]- | 430 | |
| | amino}cyclopentyl)quinazoline-2,4-diamine | | |
| 1046 | N2-((1S,3R)-3-{[2-fluoro-5-(trifluoromethyl)benzyl]amino}- | 448 | |
| | cyclopentyl)-N4,N4-dimethylquinazoline-2,4-diamine | | |
| 1047 | N4,N4-dimethyl-N2-((1S,3R)-3-{[4-(trifluoromethoxy)benzyl]- | 446 | 3 |
| | amino}cyclopentyl)quinazoline-2,4-diamine | | |
| 1048 | N2-((1S,3R)-3-{[4-bromo-2-(trifluoromethoxy)benzyl]- | 524 | 3 |
| | amino}cyclopentyl)-N4,N4-dimethylquinazoline-2,4-diamine | | |
| 1049 | N2-{(1S,3R)-3-[(3,4-difluorobenzyl)amino]cyclopentyl}-N4,N4- | 398 | 3 |
| | dimethylquinazoline-2,4-diamine | | |
| 1050 | N-(cis-4-{[4-(dimethylamino)quinazolin-2-yl]amino}cyclohexyl)- | 501 | 1 |
| | 6-(2-fluorophenoxy)nicotinamide | | |
| 1051 | 6-(2-chlorophenoxy)-N-(cis-4-{[4-(dimethylamino)quinazolin-2- | 517 | 3 |
| | yl]amino}cyclohexyl)nicotinamide | | |
| 1052 | 6-(2-bromophenoxy)-N-(cis-4-{[4-(dimethylamino)quinazolin-2- | 561 | 3 |
| | yl]amino}cyclohexyl)nicotinamide | | |
| 1053 | N-(cis-4-{[4-(dimethylamino)quinazolin-2-yl]amino}cyclohexyl)- | 498 | |
| | 6-(2-methylphenoxy)nicotinamide | | |
| 1054 | N-(cis-4-{[4-(dimethylamino)quinazolin-2-yl]amino}cyclohexyl)- | 513 | 1 |
| | 6-(2-methoxyphenoxy)nicotinamide | | |
| 1055 | N-(cis-4-{[4-(dimethylamino)quinazolin-2-yl]amino}cyclohexyl)- | 498 | 1 |
| | 6-(3-methylphenoxy)nicotinamide | | |
| 1056 | N-(cis-4-{[4-(dimethylamino)quinazolin-2-yl]amino}cyclohexyl)- | 513 | 1 |
| | 6-(3-methoxyphenoxy)nicotinamide | | |
| 1057 | N-(cis-4-{[4-(dimethylamino)quinazolin-2-yl]amino}cyclohexyl)- | 551 | 1 |
| | 6-[3-(trifluoromethyl)phenoxy]nicotinamide | | |
| 1058 | N-(cis-4-{[4-(dimethylamino)quinazolin-2-yl]amino}cyclohexyl)- | 568 | |
| | 6-[3-(trifluoromethoxy)phenoxy]nicotinamide | | |
| 1059 | N-(cis-4-{[4-(dimethylamino)quinazolin-2-yl]amino}cyclohexyl)- | 501 | 3 |
| | 6-(4-fluorophenoxy)nicotinamide | | |
| 1060 | 6-(4-chlorophenoxy)-N-(cis-4-{[4-(dimethylamino)quinazolin-2- | 518 | 1 |
| | yl]amino}cyclohexyl)nicotinamide | | |
| 1061 | 6-(4-bromophenoxy)-N-(cis-4-{[4-(dimethylamino)quinazolin-2- | 561 | 3 |
| | yl]amino}cyclohexyl)nicotinamide | | |
| 1062 | N-(cis-4-{[4-(dimethylamino)quinazolin-2-yl]amino}cyclohexyl)- | 567 | 3 |
| | 6-[4-(trifluoromethoxy)phenoxy]nicotinamide | | |
| 1063 | 6-(3,5-difluorophenoxy)-N-(cis-4-{[4-(dimethylamino)quinazolin- | 519 | 3 |
| | 2-yl]amino}cyclohexyl)nicotinamide | | |
| 1064 | 6-(2,3-difluorophenoxy)-N-(cis-4-{[4-(dimethylamino)quinazolin- | 519 | 1 |
| | 2-yl]amino}cyclohexyl)nicotinamide | | |
| 1065 | 6-(3,4-difluorophenoxy)-N-(cis-4-{[4-(dimethylamino)quinazolin- | 519 | 1 |
| | 2-yl]amino}cyclohexyl)nicotinamide | | |
| 1066 | 6-(2,3-dimethoxyphenoxy)-N-(cis-4-{[4- | 543 | 1 |
| | (dimethylamino)quinazolin-2-yl]amino}cyclohexyl)nicotinamide | | |
| 1067 | N-(cis-4-{[4-(dimethylamino)quinazolin-2-yl]amino}cyclohexyl)- | 574 | 2 |
| | 6-(3,4,5-trimethoxyphenoxy)nicotinamide | | |
| 1068 | 6-(4-chloro-2-methoxyphenoxy)-N-(cis-4-{[4- | 548 | 2 |
| | (dimethylamino)quinazolin-2-yl]amino}cyclohexyl)nicotinamide | | |
| 1069 | 6-(4-chloro-3-fluorophenoxy)-N-(cis-4-{[4- | 535 | 1 |
| | (dimethylamino)quinazolin-2-yl]amino}cyclohexyl)nicotinamide | | |
| 1070 | 6-(3-chloro-4-fluorophenoxy)-N-(cis-4-{[4- | 535 | 2 |
| | (dimethylamino)quinazolin-2-yl]amino}cyclohexyl)nicotinamide | | |
| 1071 | 6-(3,5-dimethoxyphenoxy)-N-(cis-4-{[4- | 543 | 2 |
| | (dimethylamino)quinazolin-2-yl]amino}cyclohexyl)nicotinamide | | |
| 1072 | 6-(3-bromophenoxy)-N-(cis-4-{[4-(dimethylamino)quinazolin-2- | 561 | 3 |
| | yl]amino}cyclohexyl)nicotinamide | | |
| 1073 | 2-(3-chlorophenoxy)-N-(cis-4-{[4-(dimethylamino)quinazolin-2- | 517 | 2 |
| | yl]amino}cyclohexyl)isonicotinamide | | |
| 1074 | 2-(4-chlorophenoxy)-N-(cis-4-{[4-(dimethylamino)quinazolin-2- | 517 | 1 |
| | yl]amino}cyclohexyl)isonicotinamide | | |
| 1075 | 2-(3,4-dichlorophenoxy)-N-(cis-4-{[4- | 488.2 | 1 |
| | (dimethylamino)quinazolin-2-yl]amino}cyclohexyl)acetamide | | |
| 1076 | N-(cis-4-{[4-(dimethylamino)quinazolin-2-yl]amino}cyclohexyl)- | 448.4 | 3 |
| | 2-(3,4-dimethylphenoxy)acetamide | | |
| 1077 | N-(cis-4-{[4-(dimethylamino)quinazolin-2-yl]amino}cyclohexyl)- | 524.2 | 2 |
| | 2-(2,4,5-trichlorophenoxy)acetamide | | |
| 1078 | N-(cis-4-{[4-(dimethylamino)quinazolin-2-yl]amino}cyclohexyl)- | 438.2 | 1 |
| | 2-(4-fluorophenoxy)acetamide | | |
| 1079 | N-(cis-4-{[4-(dimethylamino)quinazolin-2-yl]amino}cyclohexyl)- | 434.2 | 1 |
| | 2-(3-methylphenoxy)acetamide | | |
| 1080 | N-(cis-4-{[4-(dimethylamino)quinazolin-2-yl]amino}cyclohexyl)- | 450.2 | 1 |
| | 2-(4-methoxyphenoxy)acetamide | | |
| 1081 | N-(cis-4-{[4-(dimethylamino)quinazolin-2-yl]amino}cyclohexyl)- | 450.2 | 1 |
| | 2-(3-methoxyphenoxy)acetamide | | |
| 1082 | N-(cis-4-{[4-(dimethylamino)quinazolin-2-yl]amino}cyclohexyl)- | 450.2 | 2 |
| | 2-(2-methoxyphenoxy)acetamide | | |
| 1083 | 2-(2,4-dichlorophenoxy)-N-(cis-4-{[4- | 488.2 | 2 |
| | (dimethylamino)quinazolin-2-yl]amino}cyclohexyl)acetamide | | |
| 1084 | 4-(benzyloxy)-N-(cis-4-{[4-(dimethylamino)quinazolin-2- | 496.5 | 3 |
| | yl]amino}cyclohexyl)benzamide | | |
| 1085 | N-(cis-4-{[4-(dimethylamino)quinazolin-2-yl]amino}cyclohexyl)- | 419.4 | 1 |
| | N2-phenylglycinamide | | |
| 1086 | N-(cis-4-{[4-(dimethylamino)quinazolin-2-yl]amino}cyclohexyl)- | 433.4 | 1 |
| | N2-(3-methylphenyl)glycinamide | | |
| 1087 | N-(cis-4-{[4-(dimethylamino)quinazolin-2-yl]amino}cyclohexyl)- | 437.4 | 1 |
| | N2-(3-fluorophenyl)glycinamide | | |
| 1088 | N-(cis-4-{[4-(dimethylamino)quinazolin-2-yl]amino}cyclohexyl)- | 433.4 | 1 |
| | N2-methyl-N2-phenylglycinamide | | |
| 1089 | N2-(4-chlorophenyl)-N-(cis-4-{[4-(dimethylamino)quinazolin-2- | 453.2 | 1 |
| | yl]amino}cyclohexyl)glycinamide | | |
| 1090 | N-(cis-4-{[4-(dimethylamino)quinazolin-2-yl]amino}cyclohexyl)- | 449.2 | 1 |
| | N2-(3-methoxyphenyl)glycinamide | | |
| 1091 | N-(cis-4-{[4-(dimethylamino)quinazolin-2-yl]amino}cyclohexyl)- | 437.2 | 1 |
| | N2-(4-fluorophenyl)glycinamide | | |
| 1092 | 2-(2,6-difluorophenyl)-N-[(cis-4-{[4-(dimethylamino)quinazolin- | 470 | 3 |
| | 2-yl]amino}cyclohexyl)methyl]-2-hydroxyacetamide | | |
| 1093 | 2-(2,3-difluorophenyl)-N-[(cis-4-{[4-(dimethylamino)quinazolin- | 470 | 3 |
| | 2-yl]amino}cyclohexyl)methyl]-2-hydroxyacetamide | | |
| 1094 | 2-(2,5-difluorophenyl)-N-[(cis-4-{[4-(dimethylamino)quinazolin- | 470 | 3 |
| | 2-yl]amino}cyclohexyl)methyl]-2-hydroxyacetamide | | |
| 1095 | 2-(3,4-difluorophenyl)-N-[(cis-4-{[4-(dimethylamino)quinazolin- | 470 | 3 |
| | 2-yl]amino}cyclohexyl)methyl]-2-hydroxyacetamide | | |
| 1096 | N-[(cis-4-{[4-(dimethylamino)quinazolin-2-yl]amino}- | 464 | 2 |
| | cyclohexyl)-methyl]-2-hydroxy-2-(4-methoxyphenyl)acetamide | | |
| 1097 | N-[(cis-4-{[4-(dimethylamino)quinazolin-2-yl]amino}- | 464 | |
| | cyclohexyl)-methyl]-2-hydroxy-2-(3-methoxyphenyl)acetamide | | |
| 1098 | 2-(4-bromophenyl)-N-[(cis-4-{[4-(dimethylamino)quinazolin-2- | 512 | 3 |
| | yl]amino}cyclohexyl)methyl]-2-hydroxyacetamide | | |
| 1099 | 2-(4-chlorophenyl)-N-[(cis-4-{[4-(dimethylamino)quinazolin-2- | 468 | 3 |
| | yl]amino}cyclohexyl)methyl]-2-hydroxyacetamide | | |
| 1100 | (2S)-2-(3-chlorophenyl)-N-[(cis-4-{[4-(dimethylamino)- | 468 | 2 |
| | quinazolin-2-yl]amino}cyclohexyl)methyl]-2-hydroxyacetamide | | |
| 1101 | (2R)-2-(2-chlorophenyl)-N-[(cis-4-{[4-(dimethylamino)- | 468 | 2 |
| | quinazolin-2-yl]amino}cyclohexyl)methyl]-2-hydroxyacetamide | | |
| 1102 | (2R)-N-[(cis-4-{[4-(dimethylamino)quinazolin-2- | 434 | 3 |
| | yl]amino}cyclohexyl)methyl]-2-hydroxy-2-phenylacetamide | | |
| 1103 | (2S)-N-[(cis-4-{[4-(dimethylamino)quinazolin-2- | 434 | 3 |
| | yl]amino}cyclohexyl)methyl]-2-hydroxy-2-phenylacetamide | | |
| 1104 | N-[(cis-4-{[4-(dimethylamino)quinazolin-2-yl]amino}- | 502 | 2 |
| | cyclohexyl)-methyl]-2-hydroxy-2-[3-(trifluoromethyl)phenyl]- | | |
| | acetamide | | |
| 1105 | N-(cis-4-{[4-(dimethylamino)quinazolin-2-yl]amino}cyclohexyl)- | 450.00 | 1 |
| | 2-hydroxy-2-(4-methoxyphenyl)acetamide | | |
| 1106 | 2-(4-chlorophenyl)-N-(cis-4-{[4-(dimethylamino)quinazolin-2- | 454.20 | 2 |
| | yl]amino}cyclohexyl)-2-hydroxyacetamide | | |
| 1107 | 2-(4-bromophenyl)-N-(cis-4-{[4-(dimethylamino)quinazolin-2- | 498.40 | 2 |
| | yl]amino}cyclohexyl)-2-hydroxyacetamide | | |
| 1108 | 2-(3,4-difluorophenyl)-N-(cis-4-{[4-(dimethylamino)quinazolin- | 456.20 | 2 |
| | 2-yl]amino}cyclohexyl)-2-hydroxyacetamide | | |
| 1109 | 2-(2,3-difluorophenyl)-N-(cis-4-{[4-(dimethylamino)quinazolin- | 456.20 | 3 |
| | 2-yl]amino}cyclohexyl)-2-hydroxyacetamide | | |
| 1110 | 2-(2,6-difluorophenyl)-N-(cis-4-{[4-(dimethylamino)quinazolin- | 456.20 | 3 |
| | 2-yl]amino}cyclohexyl)-2-hydroxyacetamide | | |
| 1111 | (2R)-2-(3-chlorophenyl)-N-(cis-4-{[4-(dimethylamino)- | 454.20 | 1 |
| | quinazolin-2-yl]amino}cyclohexyl)-2-hydroxyacetamide | | |
| 1112 | N-(cis-4-{[4-(dimethylamino)quinazolin-2-yl]amino}cyclohexyl)- | 488.20 | 1 |
| | 2-hydroxy-2-[3-(trifluoromethyl)phenyl]acetamide | | |
| 1113 | N-(cis-4-{[4-(dimethylamino)quinazolin-2-yl]amino}cyclohexyl)- | 450.20 | 1 |
| | 2-hydroxy-2-(3-methoxyphenyl)acetamide | | |
| 1114 | (2S)-2-(2-chlorophenyl)-N-(cis-4-{[4-(dimethylamino)quinazolin- | 454.50 | 1 |
| | 2-yl]amino}cyclohexyl)-2-hydroxyacetamide | | |
| 1115 | 2-(2,5-difluorophenyl)-N-(cis-4-{[4-(dimethylamino)quinazolin- | 456.30 | 2 |
| | 2-yl]amino}cyclohexyl)-2-hydroxyacetamide | | |
| 1116 | cis-4-[(4-isopropylquinazolin-2-yl)amino]-N-(3- | 433.3 | |
| | methoxybenzyl)cyclohexanecarboxamide | | |
| 1117 | cis-4-[(4-isopropylquinazolin-2-yl)amino]-N-(4- | 417.3 | |
| | methylbenzyl)cyclohexanecarboxamide | | |
| 1118 | cis-N-(3-fluoro-4-methylbenzyl)-4-[(4-isopropylquinazolin-2- | 435.2 | |
| | yl)amino]cyclohexanecarboxamide | | |
| 1119 | cis-N-(2,5-dichlorobenzyl)-4-[(4-isopropylquinazolin-2- | 471.3 | |
| | yl)amino]cyclohexanecarboxamide | | |
| 1120 | N-[((1R,3S)-3-{[4-(dimethylamino)quinazolin-2- | 450.4 | 1 |
| | yl]amino}cyclopentyl)methyl]-3,5-dimethoxybenzamide | | |
| 1121 | 4-chloro-N-[((1R,3S)-3-{[4-(dimethylamino)quinazolin-2- | 424.2 | 2 |
| | yl]amino}cyclopentyl)methyl]benzamide | | |
| 1122 | 3-chloro-N-[((1R,3S)-3-{[4-(dimethylamino)quinazolin-2- | 424.2 | 1 |
| | yl]amino}cyclopentyl)methyl]benzamide | | |
| 1123 | 2,4,6-trichloro-N-[((1R,3S)-3-{[4-(dimethylamino)quinazolin-2- | 492 | 1 |
| | yl]amino}cyclopentyl)methyl]benzamide | | |
| 1124 | N-[((1R,3S)-3-{[4-(dimethylamino)quinazolin-2-yl]amino}- | 476.2 | 3 |
| | cyclopentyl)methyl]-3-fluoro-4-(trifluoromethyl)benzamide | | |
| 1125 | N-[((1R,3S)-3-{[4-(dimethylamino)quinazolin-2-yl]amino}- | 476.2 | 3 |
| | cyclopentyl)methyl]-2-fluoro-4-(trifluoromethyl)benzamide | | |
| 1126 | N-[((1R,3S)-3-{[4-(dimethylamino)quinazolin-2- | 526.4 | 3 |
| | yl]amino}cyclopentyl)methyl]-2,5-bis(trifluoromethyl)benzamide | | |
| 1127 | N-[((1R,3S)-3-{[4-(dimethylamino)quinazolin-2- | 458.2 | 1 |
| | yl]amino}cyclopentyl)methyl]-3-(trifluoromethyl)benzamide | | |
| 1128 | N-[((1R,3S)-3-{[4-(dimethylamino)quinazolin-2- | 474.4 | 3 |
| | yl]amino}cyclopentyl)methyl]-4-(trifluoromethoxy)benzamide | | |
| 1129 | N-[((1R,3S)-3-{[4-(dimethylamino)quinazolin-2- | 426.2 | 2 |
| | yl]amino}cyclopentyl)methyl]-2,5-difluorobenzamide | | |
| 1130 | N-[((1R,3S)-3-{[4-(dimethylamino)quinazolin-2- | 426.2 | 1 |
| | yl]amino}cyclopentyl)methyl]-3,5-difluorobenzamide | | |
| 1131 | N2-((1S,3R)-3-{[(3,5-dichlorobenzyl)amino]methyl}- | 444 | |
| | cyclopentyl)-N4,N4-dimethylquinazoline-2,4-diamine | | |
| 1132 | N-[(1S,3R)-3-({[4-(dimethylamino)quinazolin-2- | 408.2 | 3 |
| | yl]amino}methyl)cyclopentyl]-3-fluorobenzamide | | |
| 1133 | N-[(1S,3R)-3-({[4-(dimethylamino)quinazolin-2- | 426.2 | 3 |
| | yl]amino}methyl)cyclopentyl]-2,4-difluorobenzamide | | |
| 1134 | N4,N4-dimethyl-N2-[((1R,3S)-3-{[3-(trifluoromethyl)benzyl]- | 444 | |
| | amino}cyclopentyl)methyl]quinazoline-2,4-diamine | | |
| 1135 | cis-N-benzyl-4-{[4-(dimethylamino)quinazolin-2- | 404.3 | 1 |
| | yl]amino}cyclohexanecarboxamide | | |
| 1136 | cis-N-[(6-chloropyridin-3-yl)methyl]-4-{[4- | 439.3 | 1 |
| | (dimethylamino)quinazolin-2-yl]amino}cyclohexanecarboxamide | | |
| 1137 | cis-4-{[4-(dimethylamino)quinazolin-2-yl]amino}-N-[(1R)-1-(3- | 448.3 | 1 |
| | methoxyphenyl)ethyl]cyclohexanecarboxamide | | |
| 1138 | cis-4-{[4-(dimethylamino)quinazolin-2-yl]amino}-N-[1-(4- | 436.3 | 1 |
| | fluorophenyl)ethyl]cyclohexanecarboxamide | | |
| 1139 | cis-N-[(1R)-1-(4-chlorophenyl)ethyl)-4-{[4- | 452.3 | 1 |
| | (dimethylamino)quinazolin-2-yl]amino}cyclohexanecarboxamide | | |
| 1140 | cis-N-[1-(4-bromophenyl)ethyl]-4-{[4- | 496.4 | 1 |
| | (dimethylamino)quinazolin-2-yl]amino}cyclohexanecarboxamide | | |
| 1141 | cis-4-{[4-(dimethylamino)quinazolin-2-yl]amino}-N-[(1S)-1-(1- | 468.7 | 1 |
| | naphthyl)ethyl]cyclohexanecarboxamide | | |
| 1142 | cis-4-{[4-(dimethylamino)quinazolin-2-yl]amino}-N-(3,5- | 432.4 | 1 |
| | dimethylbenzyl)cyclohexanecarboxamide | | |
| 1143 | cis-4-{[4-(dimethylamino)quinazolin-2-yl]amino}-N-(3-fluoro-4- | 436.4 | 2 |
| | methylbenzyl)cyclohexanecarboxamide | | |
| 1144 | cis-N-(3-chloro-2-methylbenzyl)-4-{[4- | 452.2 | 1 |
| | (dimethylamino)quinazolin-2-yl]amino}cyclohexanecarboxamide | | |
| 1145 | cis-N-(5-chloro-2-methylbenzyl)-4-{[4- | 452.2 | 2 |
| | (dimethylamino)quinazolin-2-yl]amino}cyclohexanecarboxamide | | |
| 1146 | cis-4-{[4-(dimethylamino)quinazolin-2-yl]amino}-N-(5-fluoro-2- | 436.4 | 1 |
| | methylbenzyl)cyclohexanecarboxamide | | |
| 1147 | cis-N-(3-chloro-2,6-difluorobenzyl)-4-{[4- | 474.4 | 1 |
| | (dimethylamino)quinazolin-2-yl]amino}cyclohexanecarboxamide | | |
| 1148 | cis-N-(biphenyl-3-ylmethyl)-4-{[4-(dimethylamino)quinazolin-2- | 480.2 | 1 |
| | yl]amino}cyclohexanecarboxamide | | |
| 1149 | cis-N-(biphenyl-4-ylmethyl)-4-{[4-(dimethylamino)quinazolin-2- | 480.2 | |
| | yl]amino}cyclohexanecarboxamide | | |
| 1150 | cis-N-(6-chloro-2-fluoro-3-methylbenzyl)-4-{[4- | 470.4 | 1 |
| | (dimethylamino)quinazolin-2-yl]amino}cyclohexanecarboxamide | | |
| 1151 | cis-4-{[4-(dimethylamino)quinazolin-2-yl]amino}-N-(2- | 422.2 | 1 |
| | fluorobenzyl)cyclohexanecarboxamide | | |
| 1152 | cis-N-(2,6-difluorobenzyl)-4-{[4-(dimethylamino)quinazolin-2- | 440.4 | 1 |
| | yl]amino}cyclohexanecarboxamide | | |
| 1153 | cis-4-{[4-(dimethylamino)quinazolin-2-yl]amino}-N-[4- | 472.4 | 1 |
| | (trifluoromethyl)benzyl]cyclohexanecarboxamide | | |
| 1154 | cis-4-{[4-dimethylamino)quinazolin-2-yl]amino}-N-(1- | 454.4 | 1 |
| | naphthylmethyl)cyclohexanecarboxamide | | |
| 1155 | cis-N-(4-chlorobenzyl)-4-{[4-(dimethylamino)quinazolin-2- | 438.2 | 1 |
| | yl]amino}cyclohexanecarboxamide | | |
| 1156 | cis-N-(3,4-dichlorobenzyl)-4-{[4-(dimethylamino)quinazolin-2- | 472.4 | 1 |
| | yl]amino}cyclohexanecarboxamide | | |
| 1157 | cis-4-{[4-(dimethylamino)quinazolin-2-yl]amino}-N-(3- | 422.2 | 1 |
| | fluorobenzyl)cyclohexanecarboxamide | | |
| 1158 | cis-N-(2,5-difluorobenzyl)-4-{[4-(dimethylamino)quinazolin-2- | 440.4 | 1 |
| | yl]amino}cyclohexanecarboxamide | | |
| 1159 | cis-N-(2,3-difluorobenzyl)-4-{[4-(dimethylamino)quinazolin-2- | 440.4 | 1 |
| | yl]amino}cyclohexanecarboxamide | | |
| 1160 | cis-N-(3-bromobenzyl)-4-{[4-(dimethylamino)quinazolin-2- | 482.4 | 1 |
| | yl]amino}cyclohexanecarboxamide | | |
| 1161 | cis-N-(3-bromo-4-fluorobenzyl)-4-{[4- | 501.2 | 1 |
| | (dimethylamino)quinazolin-2-yl]amino}cyclohexanecarboxamide | | |
| 1162 | cis-N-(4-bromo-2-fluorobenzyl)-4-{[4- | 501.2 | 1 |
| | (dimethylamino)quinazolin-2-yl]amino}cyclohexanecarboxamide | | |
| 1163 | cis-N-(5-bromo-2-fluorobenzyl)-4-{[4- | 501.2 | 1 |
| | (dimethylamino)quinazolin-2-yl]amino}cyclohexanecarboxamide | | |
| 1164 | cis-N-(4-chloro-2-fluorobenzyl)-4-{[4- | 456.4 | 1 |
| | (dimethylamino)quinazolin-2-yl]amino}cyclohexanecarboxamide | | |
| 1165 | cis-4-{[4-(dimethylamino)quinazolin-2-yl]amino}-N-(3- | 418.2 | 1 |
| | methylbenzyl)cyclohexanecarboxamide | | |
| 1166 | cis-4-{[4-(dimethylamino)quinazolin-2-yl]amino}-N-(2- | 418.2 | 1 |
| | methylbenzyl)cyclohexanecarboxamide | | |
| 1167 | cis-4-{[4-(dimethylamino)quinazolin-2-yl]amino}-N-[2- | 488.4 | 1 |
| | (trifluoromethoxy)benzyl]cyclohexanecarboxamide | | |
| 1168 | cis-N-(2,5-difluorobenzyl)-4-{[4-(dimethylamino)quinazolin-2- | 440.4 | 1 |
| | yl]amino}cyclohexanecarboxamide | | |
| 1169 | cis-4-{[4-(dimethylamino)quinazolin-2-yl]amino}-N-(2,3,4- | 458.2 | 1 |
| | trifluorobenzyl)cyclohexanecarboxamide | | |
| 1170 | cis-4-{[4-(dimethylamino)quinazolin-2-yl]amino}-N-(2,4,5- | 458.2 | 1 |
| | trifluorobenzyl)cyclohexanecarboxamide | | |
| 1171 | cis-4-{[4-(dimethylamino)quinazolin-2-yl]amino}-N-(3,4,5- | 458.2 | 1 |
| | trifluorobenzyl)cyclohexanecarboxamide | | |
| 1172 | cis-4-{[4-(dimethylamino)quinazolin-2-yl]amino}-N-(2,3,6- | 458.2 | 1 |
| | trifluorobenzy)cyclohexanecarboxamide | | |
| 1173 | cis-4-{[4-(dimethylamino)quinazolin-2-yl]amino}-N-[3-fluoro-5- | 490.4 | 1 |
| | (trifluoromethyl)benzyl]cyclohexanecarboxamide | | |
| 1174 | cis-4-{[4-(dimethylamino)quinazolin-2-yl]amino}-N-[4-fluoro-2- | 490.4 | 1 |
| | (trifluoromethyl)benzyl]cyclohexanecarboxamide | | |
| 1175 | cis-4-{[4-(dimethylamino)quinazolin-2-yl]amino}-N-[2-fluoro-4- | 490.4 | 1 |
| | (trifluoromethyl)benzyl]cyclohexanecarboxamide | | |
| 1176 | cis-4-{[4-(dimethylamino)quinazolin-2-yl]amino}-N-[4-fluoro-3- | 490.4 | 1 |
| | (trifluoromethyl)benzyl]cyclohexanecarboxamide | | |
| 1177 | cis-4-{[4-(dimethylamino)quinazolin-2-yl]amino}-N-[2-fluoro-3- | 490.4 | 1 |
| | (trifluoromethyl)benzyl]cyclohexanecarboxamide | | |
| 1178 | cis-N-[4-chloro-3-(trifluoromethyl)benzyl]-4-{[4- | 506.2 | 1 |
| | (dimethylamino)quinazolin-2-yl]amino}cyclohexanecarboxamide | | |
| 1179 | cis-N-(2-chloro-6-fluorobenzyl)-4-{[4- | 456.2 | 1 |
| | (dimethylamino)quinazolin-2-yl]amino}cyclohexanecarboxamide | | |
| 1180 | cis-N-(4-chloro-2-fluorobenzyl)-4-{[4- | 456.2 | 1 |
| | (dimethylamino)quinazolin-2-yl]amino}cyclohexanecarboxamide | | |
| 1181 | cis-N-(3-chloro-4-fluorobenzyl)-4-{[4- | 456.2 | 1 |
| | (dimethylamino)quinazolin-2-yl]amino}cyclohexanecarboxamide | | |
| 1182 | cis-N-(2-chloro-4-fluorobenzyl)-4-{[4- | 456.2 | 1 |
| | (dimethylamino)quinazolin-2-yl]amino}cyclohexanecarboxamide | | |
| 1183 | cis-N-[2-chloro-5-(trifluoromethyl)benzyl]-4-{[4- | 506.2 | 1 |
| | (dimethylamino)quinazolin-2-yl]amino}cyclohexanecarboxamide | | |
| 1184 | cis-N-[2-(difluoromethoxy)benzyl]-4-{[4- | 470.4 | 1 |
| | (dimethylamino)quinazolin-2-yl]amino}cyclohexanecarboxamide | | |
| 1185 | cis-N-[3-(difluoromethoxy)benzyl]-4-{[4- | 470.4 | 1 |
| | (dimethylamino)quinazolin-2-yl]amino}cyclohexanecarboxamide | | |
| 1186 | cis-4-{[4-(dimethylamino)quinazolin-2-yl)amino}-N-[3- | 488.4 | 1 |
| | (trifluoromethoxy)benzyl]cyclohexanecarboxamide | | |
| 1187 | cis-N-(2,4-dichloro-6-methylbenzyl)-4-{[4- | 486.2 | 2 |
| | (dimethylamino)quinazolin-2-yl]amino}cyclohexanecarboxamide | | |
| 1188 | cis-N-(2,6-dimethoxybenzyl)-4-{[4-(dimethylamino)quinazolin-2- | 464.2 | 1 |
| | yl]amino}cyclohexanecarboxamide | | |
| 1189 | cis-4-{[4-(dimethylamino)quinazolin-2-yl]amino}-N-[(1R)-1- | 418.4 | 1 |
| | phenylethyl]cyclohexanecarboxamide | | |
| 1190 | cis-4-{[4-(dimethylamino)quinazolin-2-yl]amino}-N-[(1S)-1-(4- | 448.4 | 1 |
| | methoxyphenyl)ethyl]cyclohexanecarboxamide | | |
| 1191 | cis-4-{[4-(dimethylamino)quinazolin-2-yl]amino}-N-[(1R)-1-(3- | 448.4 | 1 |
| | methoxyphenyl)ethyl]cyclohexanecarboxamide | | |
| 1192 | cis-N-[bis(4-methoxyphenyl)methyl]-4-{[4- | 540.4 | 1 |
| | (dimethylamino)quinazolin-2-yl]amino}cyclohexanecarboxamide | | |
| 1193 | cis-4-{[4-(dimethylamino)quinazolin-2-yl]amino}-N-[2- | 472.4 | 1 |
| | (trifluoromethyl)benzyl]cyclohexanecarboxamide | | |
| 1194 | cis-4-{[4-(dimethylamino)quinazolin-2-yl]amino}-N-9H-fluoren- | 478.2 | 1 |
| | 9-ylcyclohexanecarboxamide | | |
| 1195 | cis-4-{[4-(dimethylamino)quinazolin-2-yl]amino}-N-[4- | 482.2 | 1 |
| | (methylsulfonyl)benzyl]cyclohexanecarboxamide | | |
| 1196 | cis-N-(6-chloropyridin-3-yl)-4-{[4-(dimethylamino)quinazolin-2- | 425.1 | 1 |
| | yl]amino}cyclohexanecarboxamide | | |
| 1197 | cis-N-(2-chloropyridin-3-yl)-4-{[4-(dimethylamino)quinazolin-2- | 425.1 | 3 |
| | yl]amino}cyclohexanecarboxamide | | |
| 1198 | cis-N-1H-benzimidazol-2-yl-4-{[4-(dimethylamino)quinazolin-2- | 430.3 | 3 |
| | yl]amino}cyclohexanecarboxamide | | |
| 1199 | cis-N-(5-bromo-4-tert-butyl-1,3-thiazol-2-yl)-4-{[4 | 531.1 | 3 |
| | (dimethylamino)quinazolin-2-yl]amino}cyclohexanecarboxamide | | |
| 1200 | N-(cis-4-{[4-(dimethylamino)quinazolin-2-yl]amino}cyclohexyl)- | 461.4 | 1 |
| | 3-(propionylamino)benzamide | | |
| 1201 | N-(cis-4-{[4-(dimethylamino)quinazolin-2-yl]amino}cyclohexyl)- | 489.4 | 2 |
| | 3-[(3-methylbutanoyl)amino]benzamide | | |
| 1202 | N-(cis-4-{(4-(dimethylamino)quinazolin-2-yl]amino}cyclohexyl)- | 489.5 | 2 |
| | 3-[(2,2-dimethylpropanoyl)amino]benzamide | | |
| 1203 | 3-[(cyclopentylcarbonyl)amino]-N-(cis-4-{[4- | 501.4 | 1 |
| | (dimethylamino)quinazolin-2-yl]amino}cyclohexyl)benzamide | | |
| 1204 | 3-(acetylamino)-N-(cis-4-{[4-(dimethylamino)quinazolin-2- | 447.4 | 1 |
| | yl]amino}cyclohexyl)benzamide | | |
| 1205 | N-(cis-4-{[4-(dimethylamino)-6,7-difluoroquinazolin-2- | 426 | 1 |
| | yl]amino}cyclohexyl)benzamide | | |
| 1206 | N-(cis-4-{[4-(dimethylamino)-6,7-difluoroquinazolin-2- | 440 | 1 |
| | yl]amino}cyclohexyl)-4-methylbenzamide | | |
| 1207 | N-(cis-4-{[4-(dimethylamino)-6,7-difluoroquinazolin-2- | 444 | 1 |
| | yl]amino}cyclohexyl)-4-fluorobenzamide | | |
| 1208 | N-(cis-4-{[4-(dimethylamino)-6,7-difluoroquinazolin-2- | 456 | 1 |
| | yl]amino}cyclohexyl)-3-methoxybenzamide | | |
| 1209 | N-(cis-4-{[4-(dimethylamino)-6,7-difluoroquinazolin-2- | 462 | 1 |
| | yl]amino}cyclohexyl)-3,4-difluorobenzamide | | |
| 1210 | N-(cis-4-{[4-(dimethylamino)-6,7-difluoroquinazolin-2- | 494 | 1 |
| | yl]amino}cyclohexyl)-3-(trifluoromethyl)benzamide | | |
| 1211 | N-(cis-4-{[4-(dimethylamino)-6,7-difluoroquinazolin-2- | 510 | 3 |
| | yl]amino}cyclohexyl)-4-(trifluoromethoxy)benzamide | | |

### Example 1212

### [cis-4-(4-Dimethylaamio-quinazolin-2-ylamino)-cyclohexyl]-carbamic acid isobutyl ester

### Step A: Synthesis of [cis-4-(4-dimethylamino-quinazolin-2-ylamino)-cyclohexyl]-carbamic acid isobutyl ester

To a solution of N²-(cis-4-amino-cyclohexyl)-N¹,N¹-dimethyl-quinazoline-2,4-diamine obtained in step E of example 1 (300 mg) in CHCl₃ (3 mL) were added Et₃N (307 μL) and isobutyl chloroformate (158 mg). The mixture was stirred at ambient temperature for 16 hr. To the reaction was added saturated aqueous NaHCO₃ and the aqueous layer was extracted with CHCl₃ (three times). The combined organic layer was dried over McgSO₄, filtered, concentrated, and purified by flash chromatography (N-silica gel, 25% to 66% EtOAc in hexane) to give [cis-4-(4-dimethylamino-quinazolin-2-ylamino)-cyclohexyl]-carbamic-acid isobutyl ester (195 mg) as a pale yellow oil.

ESI MS m/e 386, M+H⁺; ¹H NMR (300 MHz, CDCl₃) δ 0.93 (d, J=6.84 Hz, 6 H), 1.51-1.98 (m, 9 H), 3.27 (s, 6 H), 3.69 (brs, 1 H), 3.84 (d, J=6.84 Hz, 2 H), 4.04-4.20 (m, 1 H), 4.69 (brs, 1 H), 4.86-4.98 (m, 1 H), 6.98-7.08 (m, 1 H), 7.40-7.54 (m, 2 H), 7.82 (d, J=-7.93 Hz, 1 H).

### Example 1213

### 1-[cis-4-(4-Dimethylamino-quinazolin-2-ylamino)-cyclohexyl]-3-ethyl-thiourea hydrochloride

### Step A: Synthesis of 1-[cis-4-(4-dimethylamino-quinazolin-2-ylamino)-cyclohexyl]-3-ethyl-thiourea hydrochloride

To a solution of N²-(cis-4-amino-cyclohexyl)-N¹,N¹-dimethyl-quinazoline-2,4-diamine obtained in step E of example 1 (300 ing) in DMSO (3 mL) was added ethyl isothiocyanate (100 mg). The mixture was stirred at ambient temperature for 20 hr. To the reaction mixtuer was added H₂O (20 ml) and the aqueous layer was extracted with CHCl₃ (three times). The combined organic; layer was dried over MgSO₄, filtered, concentrated, and purified by flash chromatography (NH-silica gel, 50% EtOAc in hexane) to give a colorless amorphous. To a solution of the above material in EtOAc (2 mL) was added 4 M hydrogen chloride in EtOAc (10 mL). The mixture was stirred at ambient temperature for 1 hr and concentrated. A suspension of the residue in Et₂O (20 mL) was stirred at ambient temperature for 1 hr. The precipitate was collected by filtration, washed with Et₂O, and dried at 80° C. under reduced pressure to give 1-[cis-4-(4-dimethylamino-quinazolin-2-ylamino)-cyclohexyl]-3-ethyl-thiourea hydrochloride (296 mg) as a white solid. ESI MS m/e 373, M (free)+H⁺; ¹H NMR (300 MHz, DMSO-d₆) δ 1.07 (t, J=7.23 Hz, 3 H), 1.54-1.93 (m, 8 H), 3.30-3.63 (m, 8 H), 3.95-4.23 (m, 2 H), 7.28-7.57 (m, 3 H), 7.70-7.86 (m, 1 H), 8.03-8.26 (m, 2 H), 12.52 (brs 1 H).

### Example 1214

### 1-[cis-4-(4-Dimethylmamino-quinazolin-2-ylamino)-cyclohexyl]-3-(1,1-dimethyl-propyl)-thiourea hydrochloride

### Step A: Synthesis of 1-[cis-4-(4-dimethylamino-quinazolin-2-ylamino)-cyclohexyl]-3-(1,1-dimethyl-propyl)-thiourea hydrochloride

Using the procedure for the step A of example 1213, the title compound was obtained.

ES MS m/e 415, M (free)+H⁺; ¹H NMR (300 MHz, DMSO-d₆) δ 0.77 (t, J=7.5 Hz, 3 H), 1.16(s, 3 H), 1.36 (s, 3 H), 1.41-1.99 (m, 10 H), 3.4S (s, 6 H), 3.90-4.3 (m, 2 H), 7.13-7.54 (m, 3 H), 7.78 (t, J=7.5 Hz, 1 H), 8.17 (d, J=9.0 Hz, 1 H), 8.28 (brs, 1 H), 12.87 (brs, 1 H).

### Assay Procedures

### Example 1215

Assay for Determination of Constitutive Activity of Non-Endogenous GPCPs

A. Intracellular IP₃ Accumulation Assay

On day 1, cells to be tranfected can be plated onto 24 well plates. usually 1×10⁵ cells/well (although his umber can be optimized. On day 2 cells can be transfected by firstly mixing 0.25 ug DNA (e.g,, pCMV vector or pCM4V vector comprising polynucleotide enocoding receptor) in 50 ul serum free DMEM/well and 2 ul lipofectainine in 50 ul serum-free DEl,fED,l/well. The solutions are gently mined and incubated for 15-30 min at room temperature. Cells are washed with 0.5 ml PBS and 400 μl of serum free media is mixed with the transfection media and added to thie cells. The cells are then incubated for 3-4 hrs at 37° C./5%CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5%CO₂ On Day 4 the cells are washed with 0.5 ml PBS and 0.45 ml of assay medium is added containing inositol-free/serum free media 10μM pargyline 10 mM lithium chloride or 0.4 ml of assay medium and 50 ul of 10× ketanserin (ket) to final concentration of 10 μM. The cells are then incubated for 30 min at 37° C. The cells are then washed with 0.5 ml PBS and 200 ul of fresh/ice cold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with H₂O and stored at 40° C. in water.

### Example 1216

### High Throughput Functional Screening: FLIPR™

Subsequently, a functional based assay was used to confirm the lead hits, referred to as FLIPR™ (the Fluorometric Imaging Plate Reader) and FDSS6000™ (Functional Drug Screening System). This assay utilized 3 non-endozenous, constitutively active version of the MCH receptor.

The FLIPR and FDSS assays are able to detect intracellular Ca²⁺ concentration in cells, which can be utilized to assess receptor activation and determine whether a candidate compound is an, for example, antagonist, inverse agonist or agonist to a Gq-coupled receptor. The concentration of free Ca²⁺ in the cytosol of any cell is extremely low, whereas its concentration in the extracellular fluid and endoplasmic reticulum (ER) is very high. Thus, there is a large gradient tending to drive Ca²⁺ into the cytosol across both the plasma membrane and ER. The FLIPR™ and FDSS6000™ systems (Molecular Devices Corporation, HAMAMATSU Photonics K.K.) are designed to perform functional cell-based assays, such as the measurement of intracellular calcium for high-throughput screening, The measurement of fluorescent is associated with calcium release upon activation of the Gq-coupled receptors. Gi or Go coupled receptors are not as easily monitored through the FLIPR™ and FDSS6000™ systems because these G proteins do not couple with calcium siginal pathways.

Fluorometric Imaging Plate Reader system was used to allow for rapid, kinetic measurements of intracellular fluorescence in 96 well microplates (or 384 well microplates). Simultaneous measurements of fluorescence in all wells can be made by FLIPR or FDSS6000™ every second with high sensitivity and precision. These systems are ideal for measuring cell-based functional assays such as monitoring the intracellular calcium fluxes that occur within seconds after activation of the Gq coupled receptor.

Briefly, the cells are seeded into 96 well at 5.5×10⁴ cells/well with complete culture media (Dulbecco's Modified Eagle Medium with 10% fetal bovine serum, 2 mMi L-glutamine, 1 mM sodium pyruvate and 0.5 mg/ml G418, pH 7.4) for the assay next day. On the day of assay, the media is removed and the cells are incubated with 100 μl of loading buffer (4 μM Fluo4-AM in complete culture media containing 2.5 mM Probenicid, 0.5 mg/ml and 0.2% bovine serum albumin) in 5% CO₂ incubator at 37° C. for 1 hr. The loading buffer is removed, and the cells are washed with wash buffer (Hank's Balanced Salt Solution containing 2.5 mM Probenicid, 20 mM HEPES, 0.5 mg/ml and 0.2% bovine serum albumm, pH 7.4)). One hundred fifty μl of wash buffer containing various concentrations of test compound is added to the cells, and the cells are incubated in 5% CO₂ incubator at 37° C. for 3 min. Fifty μl of wash buffer containing various concentration of MCH are added to each well, and transient changes in [Ca²⁺]i evokled bee MCH are monoitored using the FLIPR, or FDSS in 96 shell plates at Ex. 488 nm and Em. 530 nm for 290 second. When antagonist activity of compound is tested, 50 nM of MCH is used.

Use of FLIPR™ and FDSS6000™ can be accomplished by following manufacturer's instruction (Molecular Device Corporation and HAMAMATSU Photonics K.K.).

Representative examples are shown below.

| Compound No. | IC₅₀ (nM) |
|---|---|
| Example 1 | 13 |
| Example 2 | 13 |
| Example 3 | 4.9 |
| Example 898 | 3.3 |
| Example 909 | 0.97 |

The results shown in the previous tables are in accordance with the classification as defined below.
Class 1: The value of percent of control at 10⁻⁷ M was less than 40% or the value of IC₅₀ was less than 50 nM.
Class 2: The value of percent of control at 10⁻⁷ M was from 40% to 60% or the value of IC₅₀ was from 50 nM to 200 nM.
Class 3: The value of percent of control at 10⁻⁷ M was more than 60% or the value of IC₅₀ was more than 200 nM.

The compounds in Examples 886 to 991 were tested and they showed IC₅₀ activities less than about 50 μM.

### Example 1217

### Peceptor Binding Assay

In addition to the methods described herein, another means for evaluating a test compound is by determining binding affinities to the MCH receptor. This type of assay generally requires a radiolabelled ligand to the MCH receptor. Absent the use of known ligands for the MCH receptor and radiolabels thereof, compounds of Formula (I) can be labelled with a radioisotope and used in an assay for evaluating the affinity of a test compound to the MCH receptor.

A radiolabelled MCH compound of Formula (I) can be used in a screening assay to identify/evaluate compounds. In general termns, a newly synthesized or identified compound (i.e., test compound) can be evaluated for its ability to reduce binding of the “radiolabelled compound of Formula (I)” to the MCH receptor. Accordingly, the ability to compete with the “radio-labelled compound of Formula (I)” or Radiolabelled MCH Ligand for the binding to the MCH receptor directly correlates to its binding affinity of the test compound to the MCH receptor.

### Assay Protocol for Determiing Receptor Binding for MCH

### A. MCH Receptor Preparation

293 cells (human kidney, ATCC), transiently transfected with 10 ug human MCH receptor and 60 ul Lipofectamine (per 15-cm dish), are grown in the dish for 24 hours (75% confluency) with a media change and removed with I 0 ml/dishi of Hepes-EDTA buffer ( 20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets can be stored at −80° C., until used in binding assay. When used in the assay, membranes are thawed on ice for 20 minutes and then 10 mL of incubation buffer (20 mM Hepes. 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

### B. Building Assay

For total binding, a total volume of 50 ul of appropriately diluted membranes (diluted in assay buffer containing 50 mM Tris HCl (pH 7.4), 10 mM MgCl₂, and 1 mM EDTA; 5-50 ug protein) is added to 96-well polyproylene microtiter plates followed by addition of 100 ul of assay buffer and 50 ul of Radiolabelled MCH Ligand. For nonspecific binding, 50 ul of assay buffer is added instead of 100 ul and an additional 50 ul of 10 mM cold MCH is added before 50 ul of Radiolabelled MCH Ligand is added. Plates are then incubated at room temperature for 60-]20 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted test compound is added to appropriate wells followed by addition of 50 μl of Radiolabelled MCH Ligand.

### C. Calculations

The test compounds are initially assayed at 1 and 0.1 μM and then at a range of concentrations chosen such that the middle dose would cause about 50% inhibition of a Radio-MCH Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs concentration of test compound.

Kᵢ is calculated by the Cheng and Prustoff transformation:
Kᵢ=IC₅₀/(1+[L]/K_{D})

wherein [L] is the concentration of a Radio-MCH Ligand used in the assay and K_{D} is the dissociation constant of a Radio-MCH Ligand determined independently under the same binding conditions.

It is intended that each of the patents, applications, printed publications, and other published documents mentioned or referred to in this specification be herein incorporated by reference in their entirety.

Those skilled in the art wvill appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

1. A compound of Formula (I): 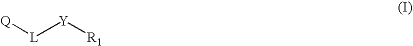
wherein Q is: 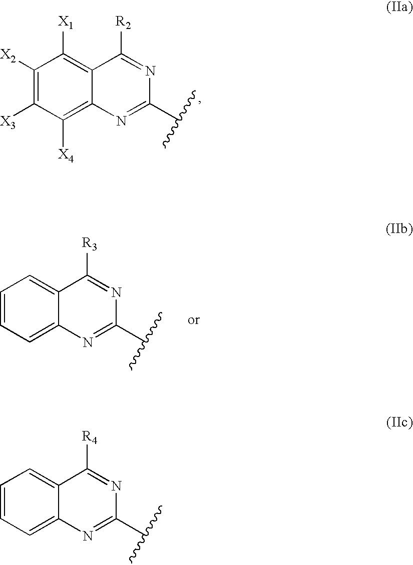
R¹ is selected from the group consisting of:
(i) C₁₋₈ alkyl, and
C₁₋₈ alkyl substituted by substituent(s) independently selected from the group consisting of:
oxo,
halogen,
C₁₋₅ alkoxy carbonyl,
C₁₋₅ alkoxy,
C₁₋₅ alkoxy substituted by carbocyclic aryl,
mono-C -5 alkylamino,
mono-C₁₋₅ alkylamino substituted by carbocyclic aryl,
di-C₁₋₅ alkylamino,
di-C₁₋₅ alkylamino substituted by carbocyclic aryl,
C₁₋₅ alkylthio,
C₃₋₆ cycloalkyl,
C₃₋₆ cycloalkyl substituted by C₁₋₅ alkyl,
C₃₋₆ cycloalkenyl,
carbocyclyl,
carbocyclic aryl,
carbocyclic aryl substituted by substituent(s) independently selected from the group consisting of:
hydroxy,
halogen,
nitro,
amino,
C₁₋₅ alkylcarbonylamino,
C₃₋₆ cycloalkylcarbonylamino,
carbocyclic aryl,
C₁₋₅ alkyl,
C₁₋₅ alkyl substituted by halogen,
C₁₋₅ alkylsulfonyl,
C₂₋₆ alkenyl,
C₁₋₅ alkoxy, and
C₁₋₅ alkoxy substituted by halogen,
mono-carbocyclic arylamino,
mono-carbocyclic arylamino substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxy substituted by halogen,
di-carbocyclic arylamino,
di-carbocyclic arylamino substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxy substituted by halogen,
carbocyclic aryloxy,
carbocyclic aryloxy substituted by substituent(s) independently selected from the group consisting of:
halogen,
C₁₋₅ alkyl,
C₁₋₅ alkyl substituted by halogen,
C₁₋₅ alkoxy,
C₁₋₅ alkoxy substituted by halogen, and
carbocyclic aryl,
hydroxy,
heterocyclyl, and
heterocyclyl substituted by halogen,
(ii) C₂₋₅ alkenyl, and C₂₋₅ alkenyl substituted by substituent(s) independently selected from the group consisting of:
oxo, and
carbocyclic aryl,
(iii) C₂₋₅ alkynyl,
(iv) C₃₋₁₂ cycloalkyl, and C₃-1₂ cycloalkyl substituted by carbocyclic aryl,
(v) carbocyclyl, and carbocyclyl substituted by substituent(s) independently selected from the group consisting of:
hydroxy, and
carbocyclic aryl,
(vi) carbocyclic aryl, and carbocyclic aryl substituted by substituent(s) independently selected from the group consisting of:
halogen,
cyano,
nitro,
amino,
C₁₋₁₀ alkyl,
C₁₋₁₀ alkyl substituted by substituent(s) independently selected from the group consisting of:
halogen,
oxo, and
carbocyclic aryl,
carboxy,
C₁₋₅ alkoxy carbonyl,
C₁₋₇ alkoxy,
C₁₋₇ alkoxy substituted by substituent(s) independently selected from the group consisting of:
halogen, and
carbocyclic aryl,
C₃₋₆ cycloalkoxy,
carbocyclic aryloxy,
carbocyclic aryloxy substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
heterocyclyloxy,
heterocyclyloxy substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
mono-C₁₋₅ alkylamino,
di-C₁₋₅ alkylamino,
C-₁₋₅ alkylcarbonylamino,
C₃₋₆ cycloalkylcarbonylamino,
C₁₋₅ alkoxy carbonylamino,
carbocyclic aryl azo,
carbocyclic aryl azo substituted by substituent(s) independently selected from the group consisting of:
mono-C₁₋₅ alkylamino, and
di-C₁₋₅ alkylamino,
C₁₋₅ alkylthio,
C₁₋₅ alkylthio substituted by halogen,
carbocyclic arylthio,
carbocyclic arylthio substituted by nitro,
amino sulfonyl,
heterocyclyl sulfonyl,
C₃₋₆ cycloalkyl,
C₃₋₆ cycloalkyl substituted by C₁₋₅ alkyl,
carbocyclic aryl,
carbocyclic aryl substituted by C₁₋₅ alkoxy,
hydroxy,
heterocyclyl, and
heterocyclyl substituted by C₁₋₅ alkyl,
(vii) heterocyclyl, and heterocyclyl substituted by substituent(s) independently selected from the group consisting of:
halogen,
C₁₋₅ alkyl,
C₁₋₅ alkyl substituted by halogen,
C₁₋₅ alkoxy,
C₁₋₅ alkoxy substituted by halogen,
C₁₋₅ alkoxy carbonyl,
C₁₋₅ alkoxy carbonyl substituted by carbocyclic aryl,
carbocyclic aryloxy,
carbocyclic aryloxy substituted by substituent(s) independently selected from the group consisting of:
halogen,
nitro,
cyano,
hydroxy,
C₁₋₅ alkyl,
C₁₋₅ alkyl substituted by halogen,
mono-C₁₋₅ alkylamino,
di-C₁₋₅ alkylamino,
C₁₋₅ alkylcarbonylamino,
C₃₋₆ cycloalkylcarbonylamino,
C₁₋₅ alkoxy,
C₁₋₅ alkoxy substituted by halogen,
C₃₋₆ cycloalkyl,
C₂₋₅ alkenyl,
C₂₋₅ alkynyl,
carboxy,
C₁₋₅ alkoxycarbonyl,
mono-C₁₋₅ alkylaminocarbonyl,
di-C₁₋₅ alkylaminocarbonyl,
mono-C₃₋₆ cycloalkylaminocarbonyl,
di-C₃₋₆ cycloalkylaminocarbonyl,
mono-C₁ 5 alkylaminocarbonylamino,
di-C₁₋₅ alkylaminocarbonylamino,
mono-C₃₋₆ cycloalkylaminocarbonylamino,
di-C₃₋₆ cycloalkylaminocarbonylamino,
C₁₋₅ alkylthio,
C₁₋₅ alkylthio substituted by halogen,
C₁₋₅ alkylsulfinyl,
C₁₋₅ alkylsulfinyl substituted by halogen,
C₁₋₅ alkylsulfonyl, and
C₁₋₅ alkylsulfonyl substituted by halogen,
heterocyclyloxy,
heterocyclyloxy substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
carbocyclic aryl, and
heterocyclyl;
R₂ is C₁₋₅ alkyl or —N(R₂ₐ)(R_{2b}); wherein R₂ₐ and R_{2b} are independently hydrogen or C₁₋₅ alkyl;
R₃ is C₁₋₅ alkyl;
R₄ is —NHNH₂, —NHNHBoc, —N(R₄ₐ)(R_{4b}), morpholino, 4-acetyl-piperazyl, or 4-phenyl-piperazyl; wherein R₄ₐ is hydrogen or C₁₋₅ alkyl; R_{4b} is C₁₋₅ alkyl, C₁₋₅ alkyl substituted by substituent(s) independently selected from the group consisting of:
hydroxy,
C₁₋₅ alkoxy,
amino,
—NHBoc,
C₃₋₆ cycloalkyl,
carbocyclic aryl,
carbocyclic aryl substituted by substituent(s) independently selected from the group consisting of:
halogen,
C₁₋₅ alkyl,
C₁₋₅ alkoxy, and
—SO₂NH₂, and
heterocyclyl,
C₃₋₆ cycloalkyl, carbocyclic aryl, carbocyclic aryl substituted by substituent(s) independently selected from the group consisting of
halogen,
C₁₋₅ alkyl,
C₁₋₅ alkoxy, and
a group of Formula (III): 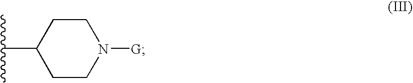
wherein Bocis carbamic acid tert-butyl ester and G is C₁₋₅ alkyl or C₁₋₅ alkyl substituted by substituent(s) independently selected from the group consisting of:
carbocyclic aryl,
halogenated carbocyclic aryl, and
carbocyclic aryl substituted by C₁₋₅ alkoxy;
L is selected from the group consisting of Formulae (IV) to (XIX): 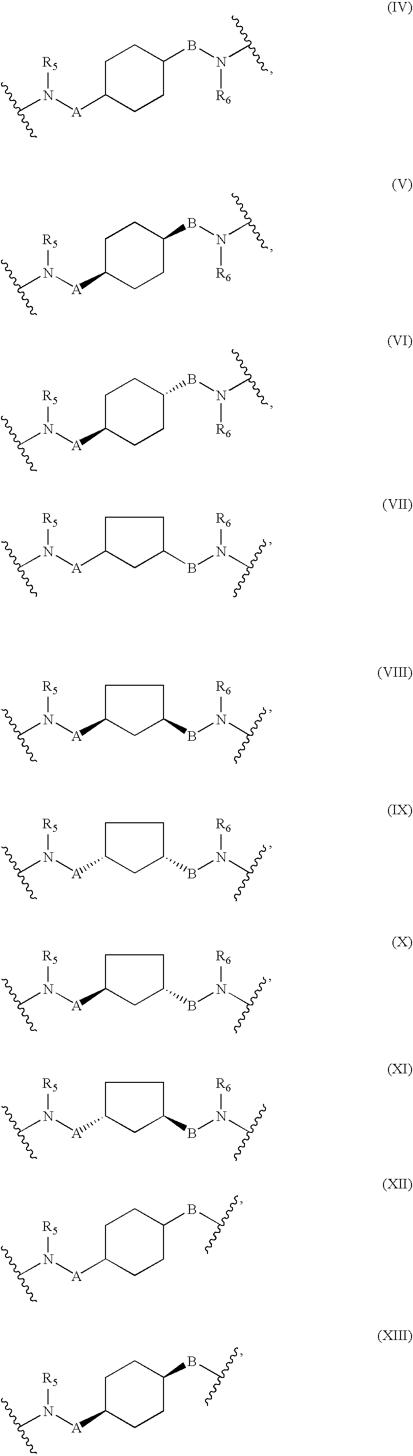 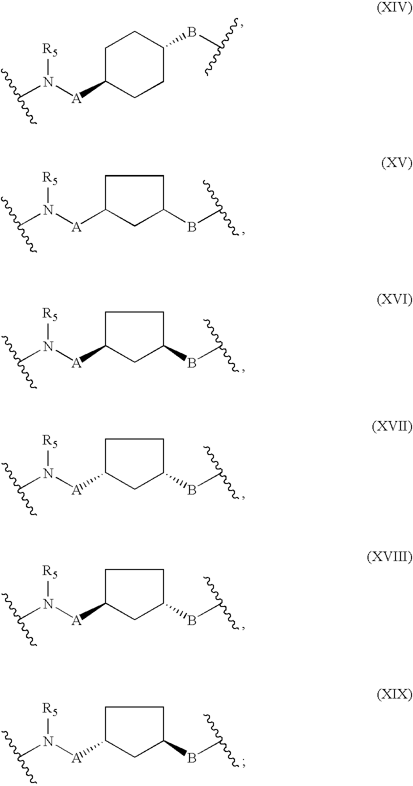
wherein R₅ and R₆ are independently hydrogen or C₁₋₅ alkyl; and A and B are independently a single bond, —CH₂—, or —(CH₂)₂—;
X₁, X₂, X₃ and X₄ are independently selected from the group consisting of hydrogen, halogen, C₁₋₄ alkyl, C₁₋₄ alkyl substituted by halogen, Cl4 alkylthio, C₁₋₄ alkylsulfinyl, C₁₋₄ alkylsulfonyl, C₁₋₄ alkoxy, C₁₋₄ alkoxy substituted by halogen, nitro, amino, mono-C₁₋₄ alkylamino, di-C₁₋₄ alkylamino, piperidyl, morpholinyl, mono-C₁₋₄ alkylaminosulfonyl, di-C₁₋₄ alkylaminosulfonyl and hydroxy; provided that at least one substituent selected from the group consisting of X₁, X₂, X₃ and X₄ is not hydrogen; and
Y is selected from the group consisting of:
(i) —C(O)NR₇—, —C(S)NR₇—, or —C(O)O— when L is selected from the group consisting of Formulae (IV) to (XIX); wherein R₇ is hydrogen or C₁₋₅ alkyl;
(ii) —S(O)₂—, —C(O)—, a single bond or —CH₂— when L is selected from the group consisting of Formulae (IV) to (XI), and Q is Formula (IIa) or (IIb);
(iii) —S(O)₂—, —C(O)—, a single bond or —CH₂— when L is selected from the group consisting of Formulae (VII) to (XI), and Q is Formula (IIc); and
(iv) —OC(O)— when L is selected from the group consisting of Formulae (XII) to (XIX);
wherein carbocyclic aryl is phenyl, naphthyl, or biphenyl;
carbocyclyl is indanyl, bicyclo[2.2.1]heptyl, bicyclo[2.2.1]heptenyl, adamantyl, 9H-fluorenyl, menthyl, 1,2,3,4-tetrahydro-naphthalen-1-yl, or 1H-indolyl;
heterocyclyl is 2,3-dihydro-benzo[1,4]dioxinyl, 3,4-dihydro-2H-benzo[b][1,4]dioxepinyl, 4,5,6,7-tetrahydro-benzo[b]thienyl, 4H-benzo[1,3]dioxinyl, benzo[1,3]dioxolyl, benzo[2,1,3]thiadiazolyl, benzothiazolyl, furyl, isoxazolyl, morpholinyl, oxazolyl, piperidyl, pyrazolyl, pyridyl, tetrahydrofuryl, thienyl, dibenzofuranyl, 1H-benzoimidazolyl, or thiazolyl; and
halogen is fluoro, chioro, bromo, or iodo;
or a pharmaceutically acceptable salt, hydrate or solvate thereof.

2. The compound according to claim 1 wherein Q is Formulae (IIa), (IIb), or (IIc);
R₁ is selected from the group consisting of:
(i) C₁₋₈ alkyl, and C₁₋₈ alkyl substituted by substituent(s) independently selected from the group consisting of:
halogen,
C₁₋₅ alkoxy carbonyl,
C₁₋₅ alkoxy,
C₁₋₅ alkoxy substituted by carbocyclic aryl,
mono-C₁₋₅ alkylamino,
di-C₁₋₅ alkylamino,
C₃₋₆ cycloalkyl,
C₃₋₆ cycloalkenyl,
carbocyclyl,
carbocyclic aryl,
carbocyclic aryl substituted by substituent(s) independently selected from the group consisting of:
hydroxy,
halogen,
nitro,
C₁₋₅ alkylcarbonylamino,
C₃₋₆ cycloalkylcarbonylamino,
C₁₋₅ alkyl,
C₁₋₅ alkyl substituted by halogen,
C₁₋₅ alkylsulfonyl,
C₂₋₆ alkenyl,
C₁₋₅ alkoxy,
C₁₋₅ alkoxy substituted by halogen, and
carbocyclic aryl,
heterocyclyl, and
heterocyclyl substituted by halogen,
(ii) C₂₋₅ alkenyl, and C₂-s alkenyl substituted by carbocyclic aryl,
(iii) C₂₋₅ alkynyl,
(iv) C₃₋₁₂ cycloalkyl, and C₃₋₁₂ cycloalkyl substituted by carbocyclic aryl,
(v) carbocyclyl, and carbocyclyl by substituent(s) independently selected from the group consisting of:
hydroxy, and
carbocyclic aryl,
(vi) carbocyclic aryl, and carbocyclic aryl substituted by substituent(s) independently selected from the group consisting of:
halogen,
cyano,
nitro,
C₁₋₁₀ alkyl,
C₁₋₁₀ alkyl substituted by substituent(s) independently selected from the group consisting of
halogen,
oxo, and
carbocyclic aryl,
carboxy,
C₁₋₅ alkoxy carbonyl,
C₁₋₇ alkoxy,
C₁₋₇ alkoxy substituted by substituent(s) independently selected from the group consisting of:
halogen, and
carbocyclic aryl,
carbocyclic aryloxy,
carbocyclic aryloxy substituted by nitro,
mono-C₁₋₅ alkylamino,
di-C₁₋₅ alkylamino,
C₁₋₅ alkoxy carbonylamino,
carbocyclic aryl azo,
carbocyclic aryl azo substituted by substituent(s) independently selected from the group consisting of:
mono-C₁₋₅ alkylamino, and
di-C₁₋₅ alkylamino,
C₁₋₅ alkylthio,
C₁₋₅ alkylthio substituted by halogen,
carbocyclic arylthio,
carbocyclic arylthio substituted by nitro,
amino sulfonyl,
heterocyclyl sulfonyl,
C₃₋₆ cycloalkyl,
C₃₋₆ cycloalkyl substituted by C₁₋₅ alkyl,
carbocyclic aryl,
heterocyclyl, and
heterocyclyl substituted by C₁₋₅ alkyl,
(vii) heterocyclyl, and heterocyclyl substituted by substituent(s) independently selected from the group consisting of:
halogen,
C₁₋₅ alkyl,
C₁₋₅ alkyl substituted by halogen,
C₁₋₅ alkoxy,
C₁₋₅ alkoxy carbonyl,
C₁₋₅ alkoxy carbonyl substituted by carbocyclic aryl,
carbocyclic aryloxy,
carbocyclic aryl, and
heterocyclyl;
R₂ is —N(R₂ₐ)(R_{2b}), wherein R₂ₐ is hydrogen or C₁₋₅ alkyl; R_{2b} is C₁₋₅ alkyl;
R₃ is C₁₋₅ alkyl;
R₄ is —N(R₄ₐ)(R_{4b}) wherein R₄a is hydrogen or C₁₋₅ alkyl; R_{4b} is C₁₋₅ alkyl;
L is selected from Formula (V), (VIII), (IX), (XIII), (XVI), or (XVII);
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is selected from the group consisting of:
(i) —C(O)NR₇—, —C(S)NR₇—, or —C(O)O— when L is selected from the group consisting of Formula (V), (VIII), (IX), (XIII), (XVI), or (XVII); wherein R₇ is hydrogen or C₁₋₅ alkyl;
(ii) —S(O)₂—, —C(O)—, a single bond or —CH₂— when L is selected from the group consisting of Formula (VIII) or (IX); and
(iii) —OC(O)— when L is selected from the group consisting of Formula (XIII), (XVI), or (XVII);
wherein carbocyclic aryl is phenyl or naphthyl;
carbocyclyl is indanyl, bicyclo[2.2.1]heptyl, bicyclo[2.2.1]heptenyl, adamantyl, 9H-fluorenyl, menthyl, 1,2,3,4-tetrahydro-naphthalen-1-yl, or 1H-indolyl;
heterocyclyl is 2,3-dihydro-benzo[1,4]dioxinyl, 3,4-dihydro-2H-benzo[b][1,4]dioxepinyl, 4,5,6,7-tetrahydro-benzo[b]thienyl, 4H-benzo[1,3]dioxinyl, benzo[1,3]dioxolyl, benzo[2,1,3]thiadiazolyl, benzothiazolyl, furyl, isoxazolyl, morpholinyl, oxazolyl, piperidyl, pyrazolyl, pyridyl, tetrahydrofuryl, thienyl, dibenzofuranyl, 1H-benzoimidazolyl, or thiazolyl; and
halogen is fluoro, chloro, bromo, or iodo;
or a pharmaceutically acceptable salt, hydrate or solvate thereof.

3. The compound according to claim 2 wherein Q is Formula (IIc);
or a pharmaceutically acceptable salt, hydrate or solvate thereof.

4. The compound according to claim 3 wherein R₁ is selected from the group consisting of:
C₁₋₅ alkyl, and C₁₋₅ alkyl substituted by substituent(s) independently selected from the group consisting of:
C₁₋₅ alkoxy carbonyl,
carbocyclic aryl,
carbocyclic aryl substituted by substituent(s) independently selected from the group consisting of:
halogen,
C₁₋₅ alkyl,
C₂₋₅ alkenyl, and
C₁₋₅ alkoxy,
C₁₋₅ alkylthio, and
heterocyclyl,
(ii) C₃₋₆ cycloalkyl, and C₃₋₆ cycloalkyl substituted by carbocyclic aryl,
(iii) carbocyclyl,
(iv) carbocyclic aryl, and carbocyclic aryl substituted by substituent(s) independently selected from the group consisting of:
halogen,
cyano,
nitro,
C₁₋₅ alkyl,
C₁₋₅ alkyl substituted by substituent(s) independently selected from the group consisting of:
halogen,
oxo, and
carbocyclic aryl,
C₁₋₅ alkoxy carbonyl,
C₁₋₇ alkoxy,
C₁₋₇ alkoxy substituted by substituent(s) independently selected from the group consisting of:
halogen, and
carbocyclic aryl,
cycloalkoxy,
carbocyclic aryloxy,
mono-C₁₋₅ alkylamino,
di-C₁₋₅ alkylamino,
C₁₋₅ alkylthio,
C₁₋₅ alkylthio substituted by halogen,
carbocyclic aryl,
heterocyclyl, and
heterocyclyl substituted by C₁₋₅ alkyl,
(v) heterocyclyl, and heterocyclyl substituted by substituent(s) independently selected from the group consisting of:
halogen,
C₁₋₅ alkyl,
C₁₋₅ alkyl substituted by halogen,
C₁₋₅ alkoxy carbonyl
C₁₋₅ alkoxy carbonyl substituted by carbocyclic aryl, and
carbocyclic aryl;
L is Formula (V); and
Y is —C(O)NR₇—; wherein R₇ is hydrogen or C₁₋₅ alkyl;
wherein carbocyclic aryl is phenyl or naphthyl;
carbocyclyl is indanyl, adamantyl, or 9H-fluorenyl;
heterocyclyl is 2,3-dihydro-benzo[1,4]dioxinyl, 3,4-dihydro-2H-benzo[b][1,4]dioxepinyl, 4H-benzo[1,3]dioxinyl, benzo[1,3]dioxolyl, benzothiazolyl, furyl, isoxazolyl, piperidyl, pyridyl, or thienyl; and
halogen is fluoro, chloro, bromo, or iodo;
or a pharmaceutically acceptable salt, hydrate or solvate thereof.

5. The compound according to claim 4 wherein R₄ₐ is hydrogen or methyl; R_{4b} is methyl; R₅ and R₆ are hydrogen; A is a single bond and B is a single bond or —CH₂—; and R₇ is hydrogen;
or a pharmaceutically acceptable salt, hydrate or solvate thereof.

6. The compound according to claim 5 wherein R₁ is selected from the group consisting of:
(i) C₁₋₅ alkyl, and
C₁₋₅ alkyl substituted by substituent(s) independently selected from the group consisting of:
C₁₋₅ alkoxy carbonyl,
carbocyclic aryl,
carbocyclic aryl substituted by substituent(s) independently selected from the group consisting of:
halogen,
C₁₋₅ alkyl,
C₂₋₅ alkenyl, and
C₁₋₅ alkoxy,
C₁₋₅ alkylthio, and
heterocyclyl,
(ii) C₃₋₆ cycloalkyl, and C₃₋₆ cycloalkyl substituted by carbocyclic aryl,
(iii) carbocyclyl,
(iv) carbocyclic aryl, and carbocyclic aryl substituted by substituent(s) independently selected from the group consisting of:
halogen,
cyano,
nitro,
C₁₋₅ alkyl,
C₁₋₅ alkyl substituted by halogen,
C₁₋₅ alkoxy carbonyl,
C₁₋₅ alkoxy,
C₁₋₅ alkoxy substituted by halogen,
cycloalkoxy,
carbocyclic aryloxy,
C₁₋₅ alkylthio, and
carbocyclic aryl,
(v) heterocyclyl, and heterocyclyl substituted by substituent(s) independently selected from the group consisting of:
halogen,
C₁₋₅ alkyl,
C₁₋₅ alkyl substituted by halogen, and
carbocyclic aryl;
wherein carbocyclic aryl is phenyl or naphthyl;
carbocyclyl is 9H-fluorenyl;
heterocyclyl is 2,3-dihydro-benzo[1,4]dioxinyl, 3,4-dihydro-2H-benzo[b][1,4]dioxepinyl, 4H-benzo[1,3]dioxinyl, benzo[1,3]dioxolyl, furyl, isoxazolyl, or thienyl; and
halogen is fluoro, chloro, bromo, or iodo;
or a pharmaceutically acceptable salt, hydrate or solvate thereof.

7. The compound according to claim 6 wherein R₁ is selected from the group consisting of:
(i) C₁₋₅ alkyl, and C₁₋₅ alkyl substituted by substituent(s) independently selected from the group consisting of:
C₁₋₅ alkoxy carbonyl,
carbocyclic aryl,
carbocyclic aryl substituted by substituent(s) independently selected from the group consisting of:
halogen,
C₁₋₅ alkyl, and
C₂₋₅ alkenyl,
C₁₋₅ alkylthio,
(ii) C₃₋₆ cycloalkyl, and C₃₋₆ cycloalkyl substituted by carbocyclic aryl,
(iii) carbocyclic aryl, and carbocyclic aryl substituted by substituent(s) independently selected from the group consisting of:
halogen,
cyano,
nitro,
C₁₋₅ alkyl,
C₁₋₅ alkyl substituted by halogen,
C₁₋₅ alkoxy carbonyl,
C₁₋₅ alkoxy,
cycloalkoxy,
carbocyclic aryloxy,
C₁₋₅ alkylthio, and
carbocyclic aryl,
(iv) heterocyclyl, and heterocyclyl substituted by substituent(s) independently selected from the group consisting of:
C₁₋₅ alkyl,
C₁₋₅ alkyl substituted by halogen, and
carbocyclic aryl;
wherein carbocyclic aryl is phenyl or naphthyl;
heterocyclyl is 2,3-dihydro-benzo[1,4]dioxinyl, 3,4-dihydro-2H-benzo[b][1,4]dioxepinyl, benzo[1,3]dioxolyl, furyl, or isoxazolyl; and
halogen is fluoro, chloro, bromo, or iodo;
or a pharmaceutically acceptable salt, hydrate or solvate thereof.

8. The compound according to claim 1 selected from the group consisting of:
N-benzyl-N′-(cis-4-{[4-(dimethylamino)quinazolin-2-yl] amino}cyclohexyl)urea;
N-(2-bromophenyl)-N′-(cis-4-{[4-(dimethylamino)quinazolin-2-yl]amino}cyclohexyl)urea;
N-biphenyl-2-yl-N′-(cis-4-{[4-(dimethylamino)quinazolin-2-yl]amino}cyclohexyl)urea;
N-(4-bromophenyl)-N′-(cis-4-{[4-(dimethylamino)quinazolin-2-yl]amino}cyclohexyl)urea;
N-butyl-N′-(cis-4-{[4-(dimethylamino)quinazolin-2-yl] amino}cyclohexyl)urea;
N-cyclohexyl-N′-(cis-4-{[4-(dimethylamino)quinazolin-2-yl] amino}cyclohexyl)urea;
N-(2-chlorophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,6-dimethylphenyl)urea;
N-(2,4-difluorophenyl)-N′-(cis-4-{[4-(dimethylamino)quinazolin-2-yl]amino}cyclohexyl)urea;
N-(2,4-dichlorophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,3-dimethylphenyl)urea;
ethyl 3-({[(cis-4-{[4-(dimethylamino)quinazolin-2-yl]amino}cyclohexyl)amino]carbonyl}amino)benzoate;
ethyl 4-({[(cis-4-{[4-(dimethylamino)quinazolin-2-yl]amino}cyclohexyl)amino]carbonyl}amino)benzoate;
N-(cis-4-{[4-(dimethylamino)quinazolin-2-yl]amino}cyclohexyl)-N′-(4-ethylphenyl)urea;
N-(cis-4-{[4-(dimethylamino)quinazolin-2-yl]amino}cyclohexyl)-N′-(2-ethyl-6-methylphenyl)urea;
ethyl N-{[(cis-4-{[4-(dimethylamino)quinazolin-2-yl]amino}cyclohexyl)amino]carbonyl}leucinate;
N-(cis-4-{[4-(dimethylamino)quinazolin-2-yl]amino}cyclohexyl)-N′-(4-fluorophenyl)urea;
N-(cis-4-{[4-(dimethylamino)quinazolin-2-yl]amino}cyclohexyl)-N′-[1-(3-isopropenylphenyl)-1-methylethyl]urea;
methyl N-{[(cis-4-{[4-(dimethylamino)quinazolin-2-yl]amino}cyclohexyl)amino]carbonyl}methioninate;
N-(cis-4-{[4-(dimethylamino)quinazolin-2-yl]amino}cyclohexyl)-N′-(4-methoxyphenyl)urea;
N-(cis-4-{[4-(dimethylamino)quinazolin-2-yl]amino}cyclohexyl)-N′-(2-methoxyphenyl)urea;
N-(cis-4-{[4-(dimethylamino)quinazolin-2-yl]amino}cyclohexyl)-N′-(3-methoxyphenyl)urea;
N-(cis-4-{[4-(dimethylamino)quinazolin-2-yl]amino}cyclohexyl)-N′-[4-(methylthio)-phenyl]urea;
N-(cis-4-{[4-(dimethylamino)quinazolin-2-yl]amino}cyclohexyl)-N′-(4-methoxybenzyl)urea;
N-(cis-4-{[4-(dimethylamino)quinazolin-2-yl]amino}cyclohexyl)-N′-1-naphthylurea;
N-(cis-4-{[4-(dimethylamino)quinazolin-2-yl]amino}cyclohexyl)-N′-[(2S)-2-phenylcyclopropyl]urea;
N-(cis-4-{[4-(dimethylamino)quinazolin-2-yl]amino}cyclohexyl)-N′-phenylurea;
N-(cis-4-{[4-(dimethylamino)quinazolin-2-yl]amino}cyclohexyl)-N′-(4-phenoxyphenyl)urea;
N-(cis-4-{[4-(dimethylamino)quinazolin-2-yl]amino}cyclohexyl)-N′-pentylurea;
N-(cis-4-{[4-(dimethylamino)quinazolin-2-yl]amino}cyclohexyl)-N′-[2-(trifluoromethyl)phenyl]urea;
N-(cis-4-{[4-(dimethylamino)quinazolin-2-yl]amino}cyclohexyl)-N′-(4-methylphenyl)urea;
N-(cis-4-{[4-(dimethylamino)quinazolin-2-yl]amino}cyclohexyl)-N′-mesitylurea;
N-(cis-4-{[4-(dimethylamino)quinazolin-2-yl]amino}cyclohexyl)-N′-(3-methylphenyl)urea;
N-(cis-4-{[4-(dimethylamino)quinazolin-2-yl]amino}cyclohexyl)-N′-(2-methylphenyl)urea;
N-(cis-4-{[4-(dimethylamino)quinazolin-2-yl]amino}cyclohexyl)-N′-[1-(1-naphthyl)ethyl]urea;
methyl N-{[(cis-4-{[4-(dimethylamino)quinazolin-2-yl]amino}cyclohexyl)amino]carbonyl}phenylalaninate;
N-(cis-4-{[4-(dimethylamino)quinazolin-2-yl]amino}cyclohexyl)-N′-(2,4,6-trichlorophenyl)urea;
N-(cis-4-{[4-(dimethylamino)quinazolin-2-yl]amino}cyclohexyl)-N′-(1-phenylethyl)urea;
1-[4-(4-dimethylamino-quinazolin-2-ylamino)-cyclohexyl]-3-(1-phenyl-ethyl)urea;
1-[4-(4-dimethylamino-quinazolin-2-ylamino)-cyclohexyl]-3-(1-naphthalen-1-yl-ethyl)urea;
N-(cis-4-{[4-(dimethylamino)quinazolin-2-yl]amino}cyclohexyl)-N′-[2-(methylthio)-phenyl]urea;
N-(cis-4-{[4-(dimethylamino)quinazolin-2-yl]amino}cyclohexyl)-N′-(2,3,5,6-tetrachlorophenyl)urea;
N-(cis-4-{[4-(dimethylamino)quinazolin-2-yl]amino}cyclohexyl)-N′-(2,3-dimethyl-6-nitrophenyl)urea;
N-(cis-4-{[4-(dimethylamino)quinazolin-2-yl]amino}cyclohexyl)-N′-(2,4,6-tribromophenyl)urea;
N-(2,4-dibromo-6-fluorophenyl)-N′-(cis-4-{[4-(dimethylamino)quinazolin-2-yl]amino}cyclohexyl)urea;
N-(2,4-dibromophenyl)-N′-(cis-4-{[4-(dimethylamino)quinazolin-2-yl]amino}cyclohexyl)urea;
N-(2,4-dichlorobenzyl)-N′-(cis-4-{[4-(dimethylamino)quinazolin-2-yl]amino}cyclohexyl)urea;
N-(2,4-dimethoxyphenyl)-N′-(cis-4-{[4-(dimethylamino)quinazolin-2-yl]amino}-cyclohexyl)urea;
N-(2,5-dimethoxyphenyl)-N′-(cis-4-{[4-(dimethylamino)quinazolin-2-yl]amino}-cyclohexyl)urea;
N-(2,6-diethylphenyl)-N′-(cis-4-{[4-(dimethylamino)quinazolin-2-yl]amino}cyclohexyl)urea;
N-(2-chloro-5-nitrophenyl)-N′-(cis-4-{[4-(dimethylamino)quinazolin-2-yl]amino}-cyclohexyl)urea;
N-[2-chloro-6-(trifluoromethyl)phenyl]-N′-(cis-4-{[4-(dimethylamino)quinazolin-2-yl]-amino}cyclohexyl)urea;
N-(2-chloro-6-methylphenyl)-N′-(cis-4-{[4-(dimethylamino)quinazolin-2-yl]amino}-cyclohexyl)urea;
N-(2-chlorobenzyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2-ethoxyphenyl)urea;
N-(cis-4-{[4-(dimethylamino)quinazolin-2-yl]amino}cyclohexyl)-N′-(2-ethyl-6-isopropylphenyl)urea;
N-(cis-4-{[4-(dimethylamino)quinazolin-2-yl]amino}cyclohexyl)-N′-(2-ethylphenyl)urea;
N-(cis-4-{[4-(dimethylamino)quinazolin-2-yl]amino}cyclohexyl)-N′-(2-fluorobenzyl)urea;
N-(cis-4-{[4-(dimethylamino)quinazolin-2-yl]amino}cyclohexyl)-N′-(2-iodophenyl)urea;
N-(cis-4-{[4-(dimethylamino)quinazolin-2-yl]amino}cyclohexyl)-N′-(2-isopropyl-6-methylphenyl)urea;
N-(cis-4-{[4-(dimethylamino)quinazolin-2-yl]amino}cyclohexyl)-N′-(2-isopropylphenyl)urea;
N-(cis-4-{[4-(dimethylamino)quinazolin-2-yl]amino}cyclohexyl)-N′-(2-methoxy-4-nitrophenyl)urea;
N-(cis-4-{[4-(dimethylamino)quinazolin-2-yl]amino}cyclohexyl)-N′-(2-methoxy-5-methylphenyl)urea;
N-(cis-4-{[4-(dimethylamino)quinazolin-2-yl]amino}cyclohexyl)-N′-(2-methyl-3-nitrophenyl)urea;
N-(cis-4-{[4-(dimethylamino)quinazolin-2-yl]amino}cyclohexyl)-N′-(2-methyl-4-nitrophenyl)urea;
N-(cis-4-{[4-(dimethylamino)quinazolin-2-yl]amino}cyclohexyl)-N′-(2-methyl-5-nitrophenyl)urea;
N-(cis-4-{[4-(dimethylamino)quinazolin-2-yl]amino}cyclohexyl)-N′-(2-methylbenzyl)urea;
N-(cis-4-{[4-(dimethylamino)quinazolin-2-yl]amino}cyclohexyl)-N′-(2-nitrophenyl)urea;
N-(cis-4-{[4-(dimethylamino)quinazolin-2-yl]amino}cyclohexyl)-N′-(2-propylphenyl)urea;
N-(cis-4-{[4-(dimethylamino)quinazolin-2-yl]amino}cyclohexyl)-N′-(2-phenoxyphenyl)urea;
N-(2-tert-butyl-6-methylphenyl)-N′-(cis-4-{[4-(dimethylamino)quinazolin-2-yl]amino}cyclohexyl)urea;
N-(2-tert-butylphenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-[3-(methylthio)-phenyl]urea;
N-1,3-benzodioxol-5-yl-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(3,4,5-trimethoxyphenyl)urea;
N-(3,4-dichlorobenzyl)-N′-(cis-4-{[4-(dimethylamino)quinazolin-2-yl]amino}cyclohexyl)urea;
N-(3,4-difluorophenyl)-N′-(cis-4-{[4-(dimethylamino)quinazolin-2-yl]amino}cyclohexyl)urea;
N-(3,4-dimethoxyphenyl)-N′-(cis-4-{[4-(dimethylamino)quinazolin-2-yl]amino}-cyclohexyl)urea;
N-(3,5-difluorophenyl)-N′-(cis-4-{[4-(dimethylamino)quinazolin-2-yl]amino}cyclohexyl)urea;
N-(3,5-dimethoxyphenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-(3,5-dimethylphenyl)urea;
methyl 3-({[(cis-4-{[4-(dimethylamino)quinazolin-2-yl]amino}cyclohexyl)amino]carbonyl}amino)benzoate;
N-(3-chloro-2-methylphenyl)-N′-(cis-4-{[4-(dimethylamino)quinazolin-2-yl]amino}-cyclohexyl)urea;
N-(3-chloro-4-fluorophenyl)-N′-(cis-4-{[4-(dimethylamino)quinazolin-2-yl]amino}-cyclohexyl)urea;
N-(3-chloro-4-methoxyphenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(3-ethylphenyl)urea;
N-(cis-4-{[4-(dimethylamino)quinazolin-2-yl]amino}cyclohexyl)-N′-(3-fluorobenzyl)urea;
N-[4-bromo-2-(trifluoromethyl)phenyl]-N′-(cis-4-{[4-(dimethylamino)quinazolin-2-yl]amino}cyclohexyl)urea;
N-(4-bromo-2,6-difluorophenyl)-N′-(cis-4-{[4-(dimethylamino)quinazolin-2-yl]amino}cyclohexyl)urea;
N-(4-bromobenzyl)-N′-(cis-4-{[4-(dimethylamino)quinazolin-2-yl]amino}cyclohexyl)urea;
N-[4-chloro-2-(trifluoromethyl)phenyl]-N′-(cis-4-{[4-(dimethylamino)quinazolin-2-yl]amino}cyclohexyl)urea;
N-(4-chloro-2-methylphenyl)-N′-(cis-4-{[4-(dimethylamino)quinazolin-2-yl]amino}-cyclohexyl)urea;
N-(4-cyanophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(4-ethoxyphenyl)urea;
N-(cis-4-{[4-(dimethylamino)quinazolin-2-yl]amino}cyclohexyl)-N′-(4-fluoro-2-nitrophenyl)urea;
N-(cis-4-{[4-(dimethylamino)quinazolin-2-yl]amino}cyclohexyl)-N′-(4-fluorobenzyl)urea;
N-(cis-4-{[4-(dimethylamino)quinazolin-2-yl]amino}cyclohexyl)-N′-(4-iodophenyl)urea;
N-(cis-4-{[4-(dimethylamino)quinazolin-2-yl]amino}cyclohexyl)-N′-(4-methoxy-2-methylphenyl)urea;
N-(cis-4-{[4-(dimethylamino)quinazolin-2-yl]amino}cyclohexyl)-N′-(4-methylbenzyl)urea;
N-(5-chloro-2,4-dimethoxyphenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(5-fluoro-2-methylphenyl)urea;
N-(cis-4-{[4-(dimethylamino)quinazolin-2-yl]amino}cyclohexyl)-N′-9H-fluoren-9-ylurea;
N-(cis-4-{[4-(dimethylamino)quinazolin-2-yl]amino}cyclohexyl)-N′-(2-phenylethyl)urea;
N-cyclopentyl-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(diphenylmethyl)urea;
N-[I -(4-bromophenyl)ethyl]-N′-(cis-4-{[4-(dimethylamino)quinazolin-2-yl]amino}-cyclohexyl)urea;
N-(4-bromo-2,6-dimethylphenyl)-N′-(cis-4-{[4-(dimethylamino)quinazolin-2-yl]amino}cyclohexyl)urea;
N-(4-bromo-2-methylphenyl)-N′-(cis-4-{[4-(dimethylamino)quinazolin-2-yl]amino}-cyclohexyl)urea;
ethyl N-{[(cis-4-{[4-(dimethylamino)quinazolin-2-yl]amino}cyclohexyl)amino]carbonyl}phenylalaninate;
N-(cis-4-{[4-(dimethylamino)quinazolin-2-yl]amino}cyclohexyl)-N′-[2-(2-thienyl)ethyl]urea;
N-(2,3-dihydro-1,4-benzodioxin-6-yl)-N′-(cis-4-{[4-(dimethylamino)quinazolin-2-yl]amino}cyclohexyl)urea;
N-(2,6-dibromo-4-isopropylphenyl)-N′-(cis-4-{[4-(dimethylamino)quinazolin-2-yl]amino}cyclohexyl)urea;
N-[3-(cyclopentyloxy)-4-methoxyphenyl]-N′-(cis-4-{[4-(dimethylamino)quinazolin-2-yl]amino}cyclohexyl)urea;
N-(3,4-dihydro-2H-1,5-benzodioxepin-7-yl)-N′-(cis-4-{[4-(dimethylamino)quinazolin-2-yl]amino}cyclohexyl)urea;
N-(4-butyl-2-methylphenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-[5-methyl-2-(trifluoromethyl)-3-furyl]urea;
N-(cis-4-{[4-(dimethylamino)quinazolin-2-yl]amino}cyclohexyl)-N′-(6-fluoro-4H-1,3-benzodioxin-8-yl)urea;
N-(cis-4-{[4-(dimethylamino)quinazolin-2-yl]amino}cyclohexyl)-N′-(3,5-dimethylisoxazol-4-yl)urea;
N-(cis-4-{[4-(dimethylamino)quinazolin-2-yl]amino}cyclohexyl)-N′-(3-methyl-5-phenylisoxazol-4-yl)urea;
N-(cis-4-{[4-(dimethylamino)quinazolin-2-yl]amino}cyclohexyl)-N′-(5-methyl-3-phenylisoxazol-4-yl)urea;
N-(2-bromophenyl)-N′-[(cis-4-{[4-(dimethylamino)quinazolin-2-yl]amino}cyclohexyl)methyl]urea;
N-biphenyl-2-yl-N′-[(cis-4-{[4-(dimethylamino)quinazolin-2-yl]amino}cyclohexyl)methyl]urea;
N-butyl-N′-[(cis-4-{[4-(dimethylamino)quinazolin-2-yl]amino}cyclohexyl)methyl]urea;
N-(3-chlorophenyl)-N′-[(cis-4-{[4-(dimethylamino)quinazolin-2-yl]amino}cyclohexyl)methyl]urea;
N-cyclohexyl-N′-[(cis-4-{[4-(dimethylamino)quinazolin-2-yl]amino}cyclohexyl)methyl]urea;
N-(3-cyanophenyl)-N′-[(cis-4-{[4-(dimethylamino)quinazolin-2-yl]amino}cyclohexyl)methyl]urea;
N-(2-chlorophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2,6-dimethylphenyl)urea;
N-(3,4-dichlorophenyl)-N′-[(cis-4-{[4-(dimethylamino)quinazolin-2-yl]amino}cyclohexyl)methyl]urea;
N-(2,4-difluorophenyl)-N′-[(cis-4-{[4-(dimethylamino)quinazolin-2-yl]amino}cyclohexyl)methyl]urea;
N-(2,4-dichlorophenyl)-N′-[(cis-4-{[4-(dimethylamino)quinazolin-2-yl]amino}cyclohexyl)methyl]urea;
N-(3,5-dichlorophenyl)-N′-[(cis-4-{[4-(dimethylamino)quinazolin-2-yl]amino}cyclohexyl)methyl]urea;
N-(2,3-dichlorophenyl)-N′-[(cis-4-{[4-(dimethylamino)quinazolin-2-yl]amino}cyclohexyl)methyl]urea;
N-(2,6-difluorophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2,3-dimethylphenyl)urea;
N-[(cis-4-{[4-(dimethylamino)quinazolin-2-yl]amino}cyclohexyl)methyl]-N′-(4-ethylphenyl)urea;
N-[(cis-4-{[4-(dimethylamino)quinazolin-2-yl]amino}cyclohexyl)methyl]-N′-(2-ethyl-6-methylphenyl)urea;
ethyl N-({[(cis-4-{[4-(dimethylamino)quinazolin-2-yl]amino}cyclohexyl)methyl]amino}carbonyl)leucinate;
N-[(cis-4-{[4-(dimethylamino)quinazolin-2-yl]amino}cyclohexyl)methyl]-N′-(4-fluorophenyl)urea;
N-[(cis-4-{[4-(dimethylamino)quinazolin-2-yl]amino}cyclohexyl)methyl]-N′-(3-fluorophenyl)urea;
N-[(cis-4-{[4-(dimethylamino)quinazolin-2-yl]amino}cyclohexyl)methyl]-N′-(2-fluorophenyl)urea;
N-[(cis-4-{[4-(dimethylamino)quinazolin-2-yl]amino}cyclohexyl)methyl]-N′-[1-(3-isopropenylphenyl)-1-methylethyl]urea;
methyl N-({[(cis-4-{[4-(dimethylamino)quinazolin-2-yl]amino}cyclohexyl)methyl]amino}carbonyl)methioninate;
N-[(cis-4-{[4-(dimethylamino)quinazolin-2-yl]amino}cyclohexyl)methyl]-N′-(4-methoxyphenyl)urea;
N-[(cis-4-{[4-(dimethylamino)quinazolin-2-yl]amino}cyclohexyl)methyl]-N′-(4-methyl-2-nitrophenyl)urea;
N-[(cis-4-{[4-(dimethylamino)quinazolin-2-yl]amino}cyclohexyl)methyl]-N′-(2-methoxyphenyl)urea;
N-[(cis-4-{[4-(dimethylamino)quinazolin-2-yl]amino}cyclohexyl)methyl]-N′-(3-methoxyphenyl)urea;
N-[(cis-4-{[4-(dimethylamino)quinazolin-2-yl]amino}cyclohexyl)methyl]-N′-[4-(methylthio)phenyl]urea;
N-[(cis-4-{[4-(dimethylamino)quinazolin-2-yl]amino}cyclohexyl)methyl]-N′-1-naphthylurea;
N-[(cis-4-{[4-(dimethylamino)quinazolin-2-yl]amino}cyclohexyl)methyl]-N′-phenylurea;
N-[(cis-4-{[4-(dimethylamino)quinazolin-2-yl]amino}cyclohexyl)methyl]-N′-pentylurea;
N-[(cis-4-{[4-(dimethylamino)quinazolin-2-yl]amino}cyclohexyl)methyl]-N′-[2-(trifluoromethyl)phenyl]urea;
N-[(cis-4-{[4-(dimethylamino)quinazolin-2-yl]amino}cyclohexyl)methyl]-N′-(4-methylphenyl)urea;
N-[(cis-4-{[4-(dimethylamino)quinazolin-2-yl]amino}cyclohexyl)methyl]-N′-mesitylurea;
N-[(cis-4-{[4-(dimethylamino)quinazolin-2-yl]amino}cyclohexyl)methyl]-N′-(3-methylphenyl)urea;
N-[(cis-4-{[4-(dimethylamino)quinazolin-2-yl]amino}cyclohexyl)methyl]-N′-(2-methylphenyl)urea;
N-[(cis-4-{[4-(dimethylamino)quinazolin-2-yl]amino}cyclohexyl)methyl]-N′-(2,4,6-trichlorophenyl)urea;
N-[(cis-4-{[4-(dimethylamino)quinazolin-2-yl]amino}cyclohexyl)methyl]-N′-(1-phenylethyl)urea;
1-[4-(4-dimethylamino-quinazolin-2-ylamino)-cyclohexylmethyl]-3-(1-phenyl-ethyl)-urea;
1-[4-(4-dimethylamino-quinazolin-2-ylamino)-cyclohexylmethyl]-3-(1-naphthalen-1-yl-ethyl)urea;
N-(2,6-diisopropylphenyl)-N′-[(cis-4-{[4-(dimethylamino)quinazolin-2-yl]amino}-cyclohexyl)methyl]urea;
N-[2-(difluoromethoxy)phenyl]-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-[2-(methylthio)phenyl]urea;
N-[(cis-4-{[4-(dimethylamino)quinazolin-2-yl]amino}cyclohexyl)methyl]-N′-(2,3,5,6-tetrachlorophenyl)urea;
N-[(cis-4-{[4-(dimethylamino)quinazolin-2-yl]amino}cyclohexyl)methyl]-N′-(2,3-dimethyl-6-nitrophenyl)urea;
N-[(cis-4-{[4-(dimethylamino)quinazolin-2-yl]amino}cyclohexyl)methyl]-N′-(2,4,6-tribromophenyl)urea;
N-(2,4-dibromo-6-fluorophenyl)-N′-[(cis-4-{[4-(dimethylamino)quinazolin-2-yl]amino}cyclohexyl)methyl]urea;
N-(2,4-dichlorobenzyl)-N′-[(cis-4-{[4-(dimethylamino)quinazolin-2-yl]amino}cyclohexyl)methyl]urea;
N-(2,5-dimethoxyphenyl)-N′-[(cis-4-{[4-(dimethylamino)quinazolin-2-yl]amino}-cyclohexyl)methyl]urea;
N-(2,6-dibromo-4-fluorophenyl)-N′-[(cis-4-{[4-(dimethylamino)quinazolin-2-yl]amino}cyclohexyl)methyl]urea;
N-(2,6-dichlorophenyl)-N′-[(cis-4-{[4-(dimethylamino)quinazolin-2-yl]amino}cyclohexyl)methyl]urea;
N-(2,6-diethylphenyl)-N′-[(cis-4-{[4-(dimethylamino)quinazolin-2-yl]amino}cyclohexyl)methyl]urea;
N-(2-chloro-5-methylphenyl)-N′-[(cis-4-{[4-(dimethylamino)quinazolin-2-yl]amino}-cyclohexyl)methyl]urea;
N-[2-chloro-6-(trifluoromethyl)phenyl]-N′-[(cis-4-{[4-(dimethylamino)quinazolin-2-yl]-amino}cyclohexyl)methyl]urea;
N-(2-chloro-6-methylphenyl)-N′-[(cis-4-{[4-(dimethylamino)quinazolin-2-yl]amino}-cyclohexyl)methyl]urea;
N-(2-chlorobenzyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2-ethyl-6-isopropylphenyl)urea;
N-[(cis-4-{[4-(dimethylamino)quinazolin-2-yl]amino}cyclohexyl)methyl]-N′-(2-ethylphenyl)urea;
N-[(cis-4-{[4-(dimethylamino)quinazolin-2-yl]amino}cyclohexyl)methyl]-N′-(2-fluoro-5-nitrophenyl)urea;
N-[(cis-4-{[4-(dimethylamino)quinazolin-2-yl]amino}cyclohexyl)methyl]-N′-(2-fluorobenzyl)urea;
N-[(cis-4-{[4-(dimethylamino)quinazolin-2-yl]amino}cyclohexyl)methyl]-N′-(2-iodophenyl)urea;
N-[(cis-4-{[4-(dimethylamino)quinazolin-2-yl]amino}cyclohexyl)methyl]-N′-(2-isopropyl-6-methylphenyl)urea;
N-[(cis-4-{[4-(dimethylamino)quinazolin-2-yl]amino}cyclohexyl)methyl]-N′-(2-isopropylphenyl)urea;
N-[(cis-4-{[4-(dimethylamino)quinazolin-2-yl]amino}cyclohexyl)methyl]-N′-(2-methoxy-5-methylphenyl)urea;
N-[(cis-4-{[4-(dimethylamino)quinazolin-2-yl]amino}cyclohexyl)methyl]-N′-(2-methoxy-5-nitrophenyl)urea;
N-[(cis-4-{[4-(dimethylamino)quinazolin-2-yl]amino}cyclohexyl)methyl]-N′-(2-methyl-3-nitrophenyl)urea;
N-[(cis-4-{[4-(dimethylamino)quinazolin-2-yl]amino}cyclohexyl)methyl]-N′-(2-methyl-4-nitrophenyl)urea;
N-[(cis-4-{[4-(dimethylamino)quinazolin-2-yl]amino}cyclohexyl)methyl]-N′-(2-methyl-5-nitrophenyl)urea;
N-[(cis-4-{[4-(dimethylamino)quinazolin-2-yl]amino}cyclohexyl)methyl]-N′-(2-methyl-6-nitrophenyl)urea;
N-[(cis-4-{[4-(dimethylamino)quinazolin-2-yl]amino}cyclohexyl)methyl]-N′-(2-methylbenzyl)urea;
N-[(cis-4-{[4-(dimethylamino)quinazolin-2-yl]amino}cyclohexyl)methyl]-N′-(2-nitrophenyl)urea;
N-[(cis-4-{[4-(dimethylamino)quinazolin-2-yl]amino}cyclohexyl)methyl]-N′-(2-propylphenyl)urea;
N-[(cis-4-{[4-(dimethylamino)quinazolin-2-yl]amino}cyclohexyl)methyl]-N′-(2-phenoxyphenyl)urea;
N-(2-tert-butyl-6-methylphenyl)-N′-[(cis-4-{[4-(dimethylamino)quinazolin-2-yl]amino}cyclohexyl)methyl]urea;
N-(2-tert-butylphenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-[3-(methylthio)phenyl]urea;
N-(3,4-difluorophenyl)-N′-[(cis-4-{[4-(dimethylamino)quinazolin-2-yl]amino}cyclohexyl)methyl]urea;
N-(3,5-difluorophenyl)-N′-[(cis-4-{[4-(dimethylamino)quinazolin-2-yl]amino}cyclohexyl)methyl]urea;
N-(3,5-dimethoxyphenyl)-N′-[(cis-4-{[4-(dimethylamino)quinazolin-2-yl]amino}-cyclohexyl)methyl]urea;
N-(3-chloro-2-methylphenyl)-N′-[(cis-4-{[4-(dimethylamino)quinazolin-2-yl]amino}-cyclohexyl)methyl]urea;
N-(3-chloro-4-fluorophenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-(3-ethylphenyl)urea;
N-[(cis-4-{[4-(dimethylamino)quinazolin-2-yl]amino}cyclohexyl)methyl]-N′-[3-fluoro-5-(trifluoromethyl)phenyl]urea;
N-[(cis-4-{[4-(dimethylamino)quinazolin-2-yl]amino}cyclohexyl)methyl]-N′-(3-fluorobenzyl)urea;
N-[(cis-4-{[4-(dimethylamino)quinazolin-2-yl]amino}cyclohexyl)methyl]-N′-(4,5-dimethyl-2-nitrophenyl)urea;
N-[4-bromo-2-(trifluoromethyl)phenyl]-N′-[(cis-4-{[4-(dimethylamino)quinazolin-2-yl]amino}cyclohexyl)methyl]urea;
N-(4-bromo-2,6-difluorophenyl)-N′-[(cis-4-{[4-(dimethylamino)quinazolin-2-yl]amino}cyclohexyl)methyl]urea;
N-[4-chloro-2-(trifluoromethyl)phenyl]-N′-[(cis-4-{[4-(dimethylamino)quinazolin-2-yl]amino}cyclohexyl)methyl]urea;
N-(4-chloro-2-methylphenyl)-N′-[(cis-4-{[4-(dimethylamino)quinazolin-2-yl]amino}-cyclohexyl)methyl]urea;
N-(4-cyanophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(4-fluoro-2-nitrophenyl)urea;
N-[(cis-4-{[4-(dimethylamino)quinazolin-2-yl]amino}cyclohexyl)methyl]-N′-(4-fluorobenzyl)urea;
N-[(cis-4-{[4-(dimethylamino)quinazolin-2-yl]amino}cyclohexyl)methyl]-N′-(4-iodophenyl)urea;
N-[(cis-4-{[4-(dimethylamino)quinazolin-2-yl]amino}cyclohexyl)methyl]-N′-(4-methoxy-2-methylphenyl)urea;
N-[(cis-4-{[4-(dimethylamino)quinazolin-2-yl]amino}cyclohexyl)methyl]-N′-(4-methyl-3-nitrophenyl)urea;
N-(5-chloro-2-methylphenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-(5-fluoro-2-methylphenyl)urea;
N-cyclopentyl-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(diphenylmethyl)urea;
N-(4-bromo-2,6-dimethylphenyl)-N′-[(cis-4-{[4-(dimethylamino)quinazolin-2-yl]amino}cyclohexyl)methyl]urea;
N-(4-bromo-2-methylphenyl)-N′-[(cis-4-{[4-(dimethylamino)quinazolin-2-yl]amino}-cyclohexyl)methyl]urea;
N-(2,6-dibromo-4-isopropylphenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-3-thienylurea;
N-[(cis-4-{[4-(dimethylamino)quinazolin-2-yl]amino}cyclohexyl)methyl]-N′-[5-methyl-2-(trifluoromethyl)-3-furyl]urea;
N-[(cis-4-{[4-(dimethylamino)quinazolin-2-yl]amino}cyclohexyl)methyl]-N′-(6-fluoro-4H-1,3-benzodioxin-8-yl)urea;
N-[(cis-4-{[4-(dimethylamino)quinazolin-2-yl]amino}cyclohexyl)methyl]-N′-(3,5-dimethylisoxazol-4-yl)urea;
N-[(cis-4-{[4-(dimethylamino)quinazolin-2-yl]amino}cyclohexyl)methyl]-N′-(3-methyl-5-phenylisoxazol-4-yl)urea;
N-[(cis-4-{[4-(dimethylamino)quinazolin-2-yl]amino}cyclohexyl)methyl]-N′-(5-methyl-3-phenylisoxazol-4-yl)urea; and
N-(cis-4-{[4-(dimethylamino)quinazolin-2-yl]amino}cyclohexyl)-N′-[3-(trifluoromethoxy)phenyl]urea;
or a pharmaceutically acceptable salt, hydrate or solvate thereof.

9. The compound according to claim 1 selected from the group consisting of:
N-(2-bromophenyl)-N′-(cis-4-{[4-(dimethylamino)quinazolin-2-yl]amino}cyclohexyl)urea;
N-biphenyl-2-yl-N′-(cis-4-{[4-(dimethylamino)quinazolin-2-yl]amino}cyclohexyl)urea;
N-butyl-N′-(cis-4-{[4-(dimethylamino)quinazolin-2-yl]amino}cyclohexyl)urea;
N-(2-chlorophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,6-dimethylphenyl)urea;
N-(2,4-difluorophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2,3-dimethylphenyl)urea;
ethyl 3-({[(cis-4-{[4-(dimethylamino)quinazolin-2-yl]amino}cyclohexyl)amino]carbonyl}amino)benzoate;
N-(cis-4-{[4-(dimethylamino)quinazolin-2-yl]amino}cyclohexyl)-N′-(2-ethyl-6-methylphenyl)urea;
ethyl N-{[(cis-4-{[4-(dimethylamino)quinazolin-2-yl]amino}cyclohexyl)amino]-carbonyl}leucinate;
N-(cis-4-{[4-(dimethylamino)quinazolin-2-yl]amino}cyclohexyl)-N′-(4-fluorophenyl)urea;
N-(cis-4-{[4-(dimethylamino)quinazolin-2-yl]amino}cyclohexyl)-N′-[1-(3-isopropenylphenyl)-1-methyl ethyl]urea;
methyl N-{[(cis-4-{[4-(dimethylamino)quinazolin-2-yl]amino}cyclohexyl)amino]-carbonyl}methioninate;
N-(cis-4-{[4-(dimethylamino)quinazolin-2-yl]amino}cyclohexyl)-N′-[4-(methylthio)phenyl]urea;
N-(cis-4-{[4-(dimethylamino)quinazolin-2-yl]amino}cyclohexyl)-N′-1-naphthylurea;
N-(cis-4-{[4-(dimethylamino)quinazolin-2-yl]amino}cyclohexyl)-N′-[(2S)-2-phenylcyclopropyl]urea;
N-(cis-4-{[4-(dimethylamino)quinazolin-2-yl]amino}cyclohexyl)-N′-(4-phenoxyphenyl)urea;
N-(cis-4-{[4-(dimethylamino)quinazolin-2-yl]amino}cyclohexyl)-N′-pentylurea;
N-(cis-4-{[4-(dimethylamino)quinazolin-2-yl]amino}cyclohexyl)-N′-[2-(trifluoromethyl)phenyl]urea;
N-(cis-4-{[4-(dimethylamino)quinazolin-2-yl]amino}cyclohexyl)-N′-mesitylurea;
N-(cis-4-{[4-(dimethylamino)quinazolin-2-yl]amino}cyclohexyl)-N′-(2-methylphenyl)urea;
N-(cis-4-{[4-(dimethylamino)quinazolin-2-yl]amino}cyclohexyl)-N′-[1-(1-naphthyl)ethyl]urea;
methyl N-{[(cis-4-{[4-(dimethylamino)quinazolin-2-yl]amino}cyclohexyl)amino]carbonyl}phenylalaninate;
N-(cis-4-{[4-(dimethylamino)quinazolin-2-yl]amino}cyclohexyl)-N′-(2,4,6-trichlorophenyl)urea;
N-(cis-4-{[4-(dimethylamino)quinazolin-2-yl]amino}cyclohexyl)-N′-(1-phenylethyl)urea;
-[4-(4-Dimethylamino-quinazolin-2-ylamino)-cyclohexyl]-3-(1-phenyl-ethyl)urea;
N-(cis-4-{[4-(dimethylamino)quinazolin-2-yl]amino}cyclohexyl)-N′-(2,3,5,6-tetrachlorophenyl)urea;
N-(cis-4-{[4-(dimethylamino)quinazolin-2-yl]amino}cyclohexyl)-N′-(2,4,6-tribromophenyl)urea;
N-(2,4-dibromo-6-fluorophenyl)-N′-(cis-4-{[4-(dimethylamino)quinazolin-2-yl]amino}cyclohexyl)urea;
N-(2,4-dibromophenyl)-N′-(cis-4-{[4-(dimethylamino)quinazolin-2-yl]amino}cyclohexyl)urea;
N-(2,4-dichlorobenzyl)-N′-(cis-4-{[4-(dimethylamino)quinazolin-2-yl]amino}cyclohexyl)urea;
N-(2,4-dimethoxyphenyl)-N′-(cis-4-{[4-(dimethylamino)quinazolin-2-yl]amino}-cyclohexyl)urea;
N-(2,6-diethylphenyl)-N′-(cis-4-{[4-(dimethylamino)quinazolin-2-yl]amino}cyclohexyl)urea;
N-[2-chloro-6-(trifluoromethyl)phenyl]-N′-(cis-4-{[4-(dimethylamino)quinazolin-2-yl]-amino}cyclohexyl)urea;
N-(2-chloro-6-methylphenyl)-N′-(cis-4-{[4-(dimethylamino)quinazolin-2-yl]amino}-cyclohexyl)urea;
N-(2-chlorobenzyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(2-ethoxyphenyl)urea;
N-(cis-4-{[4-(dimethylamino)quinazolin-2-yl]amino}cyclohexyl)-N′-(2-ethyl-6-isopropylphenyl)urea;
N-(cis-4-{[4-(dimethylamino)quinazolin-2-yl]amino}cyclohexyl)-N′-(2-ethylphenyl)urea;
N-(cis-4-{[4-(dimethylamino)quinazolin-2-yl]amino}cyclohexyl)-N′-(2-fluorobenzyl)urea;
N-(cis-4-{[4-(dimethylamino)quinazolin-2-yl]amino}cyclohexyl)-N′-(2-iodophenyl)urea;
N-(cis-4-{[4-(dimethylamino)quinazolin-2-yl]amino}cyclohexyl)-N′-(2-isopropyl-6-methylphenyl)urea;
N-(cis-4-{[4-(dimethylamino)quinazolin-2-yl]amino}cyclohexyl)-N′-(2-isopropylphenyl)urea;
N-(cis-4-{[4-(dimethylamino)quinazolin-2-yl]amino}cyclohexyl)-N′-(2-methyl-3-nitrophenyl)urea;
N-(cis-4-{[4-(dimethylamino)quinazolin-2-yl]amino}cyclohexyl)-N′-(2-methyl-4-nitrophenyl)urea;
N-(cis-4-{[4-(dimethylamino)quinazolin-2-yl]amino}cyclohexyl)-N′-(2-methyl-5-nitrophenyl)urea;
N-(cis-4-{[4-(dimethylamino)quinazolin-2-yl]amino}cyclohexyl)-N′-(2-methylbenzyl)urea;
N-(cis-4-{[4-(dimethylamino)quinazolin-2-yl]amino}cyclohexyl)-N′-(2-nitrophenyl)urea;
N-(cis-4-{[4-(dimethylamino)quinazolin-2-yl]amino}cyclohexyl)-N′-(2-propylphenyl)urea;
N-(2-tert-butyl-6-methylphenyl)-N′-(cis-4-{[4-(dimethylamino)quinazolin-2-yl]amino}cyclohexyl)urea;
N-(2-tert-butylphenyl)-N′-(cis-4-{[4-(dimethylamino)quinazolin-2-yl]amino}cyclohexyl)urea;
N-1,3-benzodioxol-5-yl-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(3,4,5-trimethoxyphenyl)urea;
N-(3,4-dimethoxyphenyl)-N′-(cis-4-{[4-(dimethylamino)quinazolin-2-yl]amino}-cyclohexyl)urea;
N-(3-chloro-2-methylphenyl)-N′-(cis-4-{[4-(dimethylamino)quinazolin-2-yl]amino}-cyclohexyl)urea;
N-(3-chloro-4-methoxyphenyl)-N′-(cis-4-{[4-(dimethylamino)quinazolin-2-yl]amino}cyclohexyl)urea;
N-[4-bromo-2-(trifluoromethyl)phenyl]-N′-(cis-4-{[4-(dimethylamino)quinazolin-2-yl]-amino}cyclohexyl)urea;
N-(4-bromo-2,6-difluorophenyl)-N′-(cis-4-{[4-(dimethylamino)quinazolin-2-yl]amino}cyclohexyl)urea;
N-(4-bromobenzyl)-N′-(cis-4-{[4-(dimethylamino)quinazolin-2-yl]amino}cyclohexyl)urea;
N-[4-chloro-2-(trifluoromethyl)phenyl]-N′-(cis-4-{[4-(dimethylamino)quinazolin-2-yl]-amino}cyclohexyl)urea;
N-(4-chloro-2-methylphenyl)-N′-(cis-4-{[4-(dimethylamino)quinazolin-2-yl]amino}-cyclohexyl)urea;
N-(4-cyanophenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(4-fluorobenzyl)urea;
N-(cis-4-{[4-(dimethylamino)quinazolin-2-yl]amino}cyclohexyl)-N′-(4-methoxy-2-methylphenyl)urea;
N-(5-chloro-2,4-dimethoxyphenyl)-N′-(cis-4-{[4-(dimethylamino)quinazolin-2-yl]amino}cyclohexyl)urea;
N-(cis-4-{[4-(dimethylamino)quinazolin-2-yl]amino}cyclohexyl)-N′-(diphenylmethyl)urea;
N-[1-(4-bromophenyl)ethyl]-N′-(cis-4-{[4-(dimethylamino)quinazolin-2-yl]amino}-cyclohexyl)urea;
N-(4-bromo-2,6-dimethylphenyl)-N′-(cis-4-{[4-(dimethylamino)quinazolin-2-yl]amino}-cyclohexyl)urea;
N-(4-bromo-2-methylphenyl)-N′-(cis-4-{[4-(dimethylamino)quinazolin-2-yl]amino}-cyclohexyl)urea;
ethyl N-{[(cis-4-{[4-(dimethylamino)quinazolin-2-yl]amino}cyclohexyl)amino]carbonyl}phenylalaninate;
N-(2,3-dihydro-1,4-benzodioxin-6-yl)-N′-(cis-4-{[4-(dimethylamino)quinazolin-2-yl]amino}cyclohexyl)urea;
N-(2,6-dibromo-4-isopropylphenyl)-N′-(cis-4-{[4-(dimethylamino)quinazolin-2-yl]amino}cyclohexyl)urea;
N-[3-(cyclopentyloxy)-4-methoxyphenyl]-N′-(cis-4-{[4-(dimethylamino)quinazolin-2-yl]amino}cyclohexyl)urea;
N-(3,4-dihydro-2H-1,5-benzodioxepin-7-yl)-N′-(cis-4-{[4-(dimethylamino)quinazolin-2-yl]amino}cyclohexyl)urea;
N-(4-butyl-2-methylphenyl)-N′-(cis-4-{[4-(dimethylamino)quinazolin-2-yl]amino}-cyclohexyl)urea;
N-(cis-4-{[4-(dimethylamino)quinazolin-2-yl]amino}cyclohexyl)-N′-[5-methyl-2-(trifluoromethyl)-3-furyl]urea;
N-(cis-4-{[4-(dimethylamino)quinazolin-2-yl]amino}cyclohexyl)-N′-(3-methyl-5-phenylisoxazol-4-yl)urea;
N-(cis-4-{[4-(dimethylamino)quinazolin-2-yl]amino}cyclohexyl)-N′-(5-methyl-3-phenylisoxazol-4-yl)urea;
N-(2-chlorophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2,6-dimethylphenyl)urea;
N-(2,4-difluorophenyl)-N′-[(cis-4-{[4-(dimethylamino)quinazolin-2-yl]amino}cyclohexyl)methyl]urea;
N-(3,5-dichlorophenyl)-N′-[(cis-4-{[4-(dimethylamino)quinazolin-2-yl]amino}cyclohexyl)methyl]urea;
N-(2,3-dichlorophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2,3-dimethylphenyl)urea;
N-[(cis-4-{[4-(dimethylamino)quinazolin-2-yl]amino}cyclohexyl)methyl]-N′-(2-ethyl-6-methylphenyl)urea;
ethyl N-({[(cis-4-{[4-(dimethylamino)quinazolin-2-yl]amino}cyclohexyl)methyl]amino}carbonyl)leucinate;
N-[(cis-4-{[4-(dimethylamino)quinazolin-2-yl]amino}cyclohexyl)methyl]-N′-(4-fluorophenyl)urea;
N-[(cis-4-{[4-(dimethylamino)quinazolin-2-yl]amino}cyclohexyl)methyl]-N′-[4-(methylthio)phenyl]urea;
N-[(cis-4-{[4-(dimethylamino)quinazolin-2-yl]amino}cyclohexyl)methyl]-N′-phenylurea;
N-[(cis-4-{[4-(dimethylamino)quinazolin-2-yl]amino}cyclohexyl)methyl]-N′-[2-(trifluoromethyl)phenyl]urea;
N-[(cis-4-{[4-(dimethylamino)quinazolin-2-yl]amino}cyclohexyl)methyl]-N′-(4-methylphenyl)urea;
N-[(cis-4-{[4-(dimethylamino)quinazolin-2-yl]amino}cyclohexyl)methyl]-N′-mesitylurea;
N-[(cis-4-{[4-(dimethylamino)quinazolin-2-yl]amino}cyclohexyl)methyl]-N′-(2-methylphenyl)urea;
N-[(cis-4-{[4-(dimethylamino)quinazolin-2-yl]amino}cyclohexyl)methyl]-N′-(2,4,6-trichlorophenyl)urea;
N-(2,6-diisopropylphenyl)-N′-[(cis-4-{[4-(dimethylamino)quinazolin-2-yl]amino}-cyclohexyl)methyl]urea;
N-[(cis-4-{[4-(dimethylamino)quinazolin-2-yl]amino}cyclohexyl)methyl]-N′-(2,3-dimethyl-6-nitrophenyl)urea;
N-[(cis-4-{[4-(dimethylamino)quinazolin-2-yl]amino}cyclohexyl)methyl]-N′-(2,4,6-tribromophenyl)urea;
N-(2,4-dibromo-6-fluorophenyl)-N′-[(cis-4-{[4-(dimethylamino)quinazolin-2-yl]amino}cyclohexyl)methyl]urea;
N-(2,6-dibromo-4-fluorophenyl)-N′-[(cis-4-{[4-(dimethylamino)quinazolin-2-yl]amino}cyclohexyl)methyl]urea;
N-(2,6-dichlorophenyl)-N′-[(cis-4-{[4-(dimethylamino)quinazolin-2-yl]amino}cyclohexyl)methyl]urea;
N-(2,6-diethylphenyl)-N′-[(cis-4-{[4-(dimethylamino)quinazolin-2-yl]amino}cyclohexyl)methyl]urea;
N-[2-chloro-6-(trifluoromethyl)phenyl]-N′-[(cis-4-{[4-(dimethylamino)quinazolin-2-yl]-amino}cyclohexyl)methyl]urea;
N-(2-chloro-6-methylphenyl)-N′-[(cis-4-{[4-(dimethylamino)quinazolin-2-yl]amino}-cyclohexyl)methyl]urea;
N-(2-chlorobenzyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(2-ethyl-6-isopropylphenyl)urea;
N-[(cis-4-{[4-(dimethylamino)quinazolin-2-yl]amino}cyclohexyl)methyl]-N′-(2-ethylphenyl)urea;
N-[(cis-4-{[4-(dimethylamino)quinazolin-2-yl]amino}cyclohexyl)methyl]-N′-(2-iodophenyl)urea;
N-[(cis-4-{[4-(dimethylamino)quinazolin-2-yl]amino}cyclohexyl)methyl]-N′-(2-isopropyl-6-methylphenyl)urea;
N-[(cis-4-{[4-(dimethylamino)quinazolin-2-yl]amino}cyclobexyl)methyl]-N′-(2-isopropylphenyl)urea;
N-[(cis-4-{[4-(dimethylamino)quinazolin-2-yl]amino}cyclohexyl)methyl]-N′-(2-methoxy-5-methylphenyl)urea;
N-[(cis-4-{[4-(dimethylamino)quinazolin-2-yl]amino}cyclohexyl)methyl]-N′-(2-methyl-3-nitrophenyl)urea;
N-[(cis-4-{[4-(dimethylamino)quinazolin-2-yl]amino}cyclohexyl)methyl]-N′-(2-methyl-6-nitrophenyl)urea; N-[(cis-4-{[4-(dimethylamino)quinazolin-2-yl]amino}cyclohexyl)methyl]-N′-(2-propylphenyl)urea;
N-(2-tert-butyl-6-methylphenyl)-N′-[(cis-4-{[4-(dimethylamino)quinazolin-2-yl]amino}cyclohexyl)methyl]urea;
N-(2-tert-butylphenyl)-N′-[(cis-4-{[4-(dimethylamino)quinazolin-2-yl]amino}cyclohexyl)methyl]urea;
N-(3,4-difluorophenyl)-N′-[(cis-4-{[4-(dimethylamino)quinazolin-2-yl]amino}cyclohexyl)methyl]urea;
N-(3,5-difluorophenyl)-N′-[(cis-4-{[4-(dimethylamino)quinazolin-2-yl]amino}cyclohexyl)methyl]urea;
N-(3-chloro-2-methylphenyl)-N′-[(cis-4-{[4-(dimethylamino)quinazolin-2-yl]amino}-cyclohexyl)methyl]urea;
N-(3-chloro-4-fluorophenyl)-N′-[(cis-4-{[4-(dimethylamino)quinazolin-2-yl]amino}-cyclohexyl)methyl]urea;
N-(4-bromo-2,6-difluorophenyl)-N′-[(cis-4-{[4-(dimethylamino)quinazolin-2-yl]amino}cyclohexyl)methyl]urea;
N-[4-chloro-2-(trifluoromethyl)phenyl]-N′-[(cis-4-{[4-(dimethylamino)quinazolin-2-yl]-amino}cyclohexyl)methyl]urea;
N-(4-cyanophenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-(diphenylmethyl)urea;
N-(4-bromo-2,6-dimethylphenyl)-N′-[(cis-4-{[4-(dimethylamino)quinazolin-2-yl]amino}cyclohexyl)methyl]urea;
N-[(cis-4-{[4-(dimethylamino)quinazolin-2-yl]amino}cyclohexyl)methyl]-N′-[5-methyl-2-(trifluoromethyl)-3-furyl]urea; and
N-[(cis-4-{[4-(dimethylamino)quinazolin-2-yl]amino}cyclohexyl)methyl]-N′-(3-methyl-5-phenylisoxazol-4-yl)urea;
or a pharmaceutically acceptable salt, hydrate or solvate thereof.

10. The compound according to claim 3 wherein R₁ is selected from the group consisting of:
(i) C₁₋₈ alkyl, and
C₁₋₈ alkyl substituted by substituent(s) independently selected from the group consisting of:
mono-C₁₋₅ alkylamino,
di-C₁₋₅ alkylamino,
C₃₋₆ cycloalkyl,
C₃₋₆ cycloalkenyl,
carbocyclic aryl,
carbocyclic aryl substituted by substituent(s) independently selected from the group consisting of:
halogen,
C₁₋₅ alkyl, and
C₁₋₅ alkoxy,
heterocyclyl,
(ii) C₂₋₅ alkynyl,
(iii) C₂₋₅ alkenyl, and C₂₋₅ alkenyl substituted by carbocyclic aryl,
(iv) C₃₋₁₂ cycloalkyl,
(v) carbocyclyl,
(vi) carbocyclic aryl, and carbocyclic aryl substituted by substituent(s) independently selected from the group consisting of:
halogen,
cyano,
nitro,
C₁₋₁₀ alkyl,
C₁₋₁₀ alkyl substituted by substituent(s) independently selected from the group consisting of:
halogen, and
oxo,
carboxy,
C₁₋₅ alkoxy carbonyl,
C₁₋₅ alkoxy,
C₁₋₅ alkoxy substituted by substituent(s) independently selected from the group consisting of:
halogen, and
carbocyclic aryl,
carbocyclic aryloxy,
carbocyclic aryloxy substituted by nitro,
mono-C₁₋₅ alkylamino,
di-C₁₋₅ alkylamino,
C₁₋₅ alkoxy carbonylamino,
carbocyclic aryl azo,
carbocyclic aryl azo substituted by substituent(s) independently selected from the group consisting of:
-mono-C₁₋₅ alkylamino, and
di-C₁₋₅ alkylamino,
C₁₋₅ alkylthio,
C₁₋₅ alkylthio substituted by halogen,
carbocyclic arylthio,
carbocyclic arylthio substituted by nitro,
amino sulfonyl,
heterocyclyl sulfonyl,
C₃₋₆ cycloalkyl,
C₃₋₆ cycloalkyl substituted by C₁₋₅ alkyl,
carbocyclic aryl, and
heterocyclyl,
(vii) heterocyclyl, and heterocyclyl substituted by substituent(s) independently selected from the group consisting of:
C₁₋₅ alkyl,
C₁₋₅ alkoxy carbonyl,
carbocyclic aryloxy,
carbocyclic aryl, and
heterocyclyl;
L is Formula (V); and
Y is —C(S)NR₇—; wherein R₇ is hydrogen or C₁₋₅ alkyl;
wherein carbocyclic aryl is phenyl or naphthyl;
carbocyclyl is indanyl, bicyclo[2.2.1]heptyl, bicyclo[2.2.1]heptenyl, or adamantyl;
heterocyclyl is 2,3-dihydro-benzo[1,4]dioxinyl, 4,5,6,7-tetrahydro-benzo[b]thienyl, benzo[1,3]dioxolyl, benzo[2,1,3]thiadiazolyl, furyl, isoxazolyl, morpholinyl, oxazolyl, piperidyl, pyrazolyl, pyridyl, tetrahydrofuryl, or thienyl; and
halogen is fluoro, chloro, bromo, or iodo;
or a pharmaceutically acceptable salt, hydrate or solvate thereof.

11. The compound according to claim 10 wherein R4a is hydrogen or methyl; R_{4b} is methyl; R₅ and R₆ are hydrogen; A is a single bond; B is a single bond or —CH₂—; and R₇ is hydrogen;
or a pharmaceutically acceptable salt, hydrate or solvate thereof.

12. The compound according to claim 11 wherein R₁ is selected from the group consisting of:
(i) C₁₋₆ alkyl, and C₁₋₆ alkyl substituted by substituent(s) independently selected from the group consisting of:
C₃₋₆ cycloalkyl,
C₃₋₆ cycloalkenyl,
carbocyclic aryl,
carbocyclic aryl substituted by substituent(s) independently selected from the group consisting of:
halogen,
C₁₋₅ alkyl, and
C₁₋₅ alkoxy,
heterocyclyl,
(ii) C₃₋₁₂ cycloalkyl,
(iii) carbocyclyl,
(iv) carbocyclic aryl, and carbocyclic aryl substituted by substituent(s) independently selected from the group consisting of:
halogen,
cyano,
nitro,
C₁₋₅ alkyl,
C₁₋₅ alkyl substituted by halogen,
C₁₋₅ alkoxy carbonyl,
C₁₋₅ alkoxy,
C₁₋₅ alkoxy substituted by halogen,
mono-C₁₋₅ alkylamino,
di-C₁₋₅ alkylamino,
C₁₋₅ alkylthio, and
carbocyclic aryl,
(v) heterocyclyl, and heterocyclyl substituted by substituent(s) independently selected from the group consisting of:
C₁₋₅ alkyl,
C₁₋₅ alkoxy carbonyl, and
carbocyclic aryl;
wherein carbocyclic aryl is phenyl or naphthyl;
carbocyclyl is indanyl, bicyclo[2.2.1]heptyl, or bicyclo[2.2.1]heptenyl;
heterocyclyl is 2,3-dihydro-benzo[1,4]dioxinyl, benzo[1,3]dioxolyl, isoxazolyl, tetrahydrofuryl, or thienyl; and
halogen is fluoro, chloro, bromo, or iodo;
or a pharmaceutically acceptable salt, hydrate or solvate thereof.

13. The compound according to claim 12 wherein R₁ is selected from the group consisting of:
(i) C₁₋₅ alkyl, and C₁₋₅ alkyl substituted by substituent(s) independently selected from the group consisting of:
carbocyclic aryl,
carbocyclic aryl substituted by substituent(s) independently selected from the group consisting of:
halogen, and
C₁₋₅ alkoxy,
(ii) carbocyclyl,
(iii) carbocyclic aryl, and carbocyclic aryl substituted by substituent(s) independently selected from the group consisting of:
halogen,
cyano,
nitro,
C₁₋₅ alkyl,
C₁₋₅ alkyl substituted by halogen,
C₁₋₅ alkoxy carbonyl,
C₁₋₅ alkoxy,
C₁₋₅ alkoxy substituted by halogen,
mono-C₁₋₅ alkylamino,
di-C₁₋₅ alkylamino, and
carbocyclic aryl,
(iv) heterocyclyl, and heterocyclyl substituted by substituent(s) independently selected from the group consisting of:
C₁₋₅ alkyl,
C₁₋₅ alkoxy carbonyl, and
carbocyclic aryl;
wherein carbocyclic aryl is phenyl or naphthyl;
carbocyclyl is bicyclo[2.2.1]heptyl;
heterocyclyl is 2,3-dihydro-benzo[1,4]dioxinyl, benzo[1,3]dioxolyl, isoxazolyl, or thienyl; and
halogen is fluoro, chloro, bromo, or iodo;
or a pharmaceutically acceptable salt, hydrate or solvate thereof.

14. The compound according to claim 1 selected from the group consisting of:
N-(4-bromophenyl)-N′-(cis-4-{[4-(dimethylamino)quinazolin-2-yl]amino}cyclohexyl)thiourea;
N-(4-cyanophenyl)-N′-(cis-4-{[4-(dimethylamino)quinazolin-2-yl]amino}cyclohexyl)thiourea;
N-cyclohexyl-N′-(cis-4-{[4-(dimethylamino)quinazolin-2-yl]amino}cyclohexyl)thiourea;
N-cyclopentyl-N′-(cis-4-{[4-(dimethylamino)quinazolin-2-yl]amino}cyclohexyl)thiourea;
N-(4-chlorophenyl)-N′-(cis-4-{[4-(dimethylamino)quinazolin-2-yl]amino}cyclohexyl)-thiourea;
N-(2,4-dichlorophenyl)-N′-(cis-4-{[4-(dimethylamino)quinazolin-2-yl]amino}cyclohexyl)thiourea;
N-(2,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2,6-dimethylphenyl)thiourea;
N-(cis-4-{[4-(dimethylamino)quinazolin-2-yla amino}cyclohexyl)-N′-(2-ethyl-6-isopropylphenyl)thiourea;
N-(cis-4-{[4-(dimethylamino)quinazolin-2-yl]amino}cyclohexyl)-N′-(4-fluorophenyl)thiourea;
N-(cis-4-{[4-(dimethylamino)quinazolin-2-yl]amino}cyclohexyl)-N′-hexylthiourea;
N-(cis-4-{[4-(dimethylamino)quinazolin-2-yl]amino}cyclohexyl)-N′-isobutylthiourea;
N-(cis-4-{[4-(dimethylamino)quinazolin-2-yl]amino}cyclohexyl)-N′-(4-methoxybiphenyl-3-yl)thiourea;
N-(1,3-benzodioxol-5-ylmethyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[4-(methylthio)phenyl]thiourea;
N-(cis-4-{[4-(dimethylamino)quinazolin-2-yl]amino}cyclohexyl)-N′-(4-methoxyphenyl)thiourea;
N-(cis-4-{[4-(dimethylamino)quinazolin-2-yl]amino}cyclohexyl)-N′-(2-methoxyphenyl)thiourea;
N-(cis-4-{[4-(dimethylamino)quinazolin-2-yl]amino}cyclohexyl)-N′-1-naphthylthiourea;
N-(cis-4-{[4-(dimethylamino)quinazolin-2-yl]amino}cyclohexyl)-N′-(4-nitrophenyl)-thiourea;
N-(cis-4-{[4-(dimethylamino)quinazolin-2-yl]amino}cyclohexyl)-N′-(pentafluorophenyl)thiourea;
N-(cis-4-{[4-(dimethylamino)quinazolin-2-yl]amino}cyclohexyl)-N′-propylthiourea;
N-(cis-4-{[4-(dimethylamino)quinazolin-2-yl]amino}cyclohexyl)-N′-(3,4,5-trimethoxyphenyl)thiourea;
N-(cis-4-{[4-(dimethylamino)quinazolin-2-yl]amino}cyclohexyl)-N′-(4-methylphenyl)thiourea;
N-(3,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(4-ethylphenyl)-thiourea;
N-(cis-4-{[4-(dimethylamino)quinazolin-2-yl]amino}cyclohexyl)-N′-[2-(methylthio)-phenyl]thiourea;
N-(cis-4-{[4-(dimethylamino)quinazolin-2-yl]amino}cyclohexyl)-N′-[2-(trifluoromethoxy)phenyl]thiourea;
N-(cis-4-{[4-(dimethylamino)quinazolin-2-yl]amino}cyclohexyl)-N′-(2,3,4-trifluorophenyl)thiourea;
N-(2,5-dimethoxyphenyl)-N′-(cis-4-{[4-(dimethylamino)quinazolin-2-yl]amino}-cyclohexyl)thiourea;
N-(2-chloro-4-nitrophenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethylphenyl)-thiourea;
N-(cis-4-{[4-(dimethylamino)quinazolin-2-yl]amino}cyclohexyl)-N′-(2-iodophenyl)-thiourea;
N-(cis-4-{[4-(dimethylamino)quinazolin-2-yl]amino}cyclohexyl)-N′-(2-methoxy-4-nitrophenyl)thiourea;
N-(cis-4-{[4-(dimethylamino)quinazolin-2-yl]amino}cyclohexyl)-N′-(2-methoxy-5-methylphenyl)thiourea;
N-(cis-4-{[4-(dimethylamino)quinazolin-2-yl]amino}cyclohexyl)-N′-(3-iodophenyl)-thiourea;
N-(cis-4-{[4-(dimethylamino)quinazolin-2-yl]amino}cyclohexyl)-N′-(3-methoxyphenyl)thiourea;
N-[4-(difluoromethoxy)phenyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[4-(trifluoromethyl)phenyl]thiourea;
N-(4-bromo-2-chlorophenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(4-iodophenyl)-thiourea;
N-(5-chloro-2-methylphenyl)-N′-(cis-4-{[4-(dimethylamino)quinazolin-2-yl]amino}-cyclohexyl)thiourea;
N-[(1S,4R)-bicyclo[2.2.1]hept-2-yl]-N′-(cis-4-{[4-(dimethylamino)quinazolin-2-yl]amino}cyclohexyl)thiourea;
N-[2-(4-chlorophenyl)ethyl]-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2,4,6-tribromophenyl)thiourea;
N-(cis-4-{[4-(dimethylamino)quinazolin-2-yl]amino}cyclohexyl)-N′-(2,4,6-trichlorophenyl)thiourea;
N-(cis-4-{[4-(dimethylamino)quinazolin-2-yl]amino}cyclohexyl)-N′-mesitylthiourea;
N-(cis-4-{[4-(dimethylamino)quinazolin-2-yl]amino}cyclohexyl)-N′-(2,4-dimethylphenyl)thiourea;
N-(2,6-diethylphenyl)-N′-(cis-4-{[4-(dimethylamino)quinazolin-2-yl]amino}cyclohexyl)thiourea;
N-(2,6-diisopropylphenyl)-N′-(cis-4-{[4-(dimethylamino)quinazolin-2-yl]amino}-cyclohexyl)thiourea;
N-(2-bromo-4-methylphenyl)-N′-(cis-4-{[4-(dimethylamino)quinazolin-2-yl]amino}-cyclohexyl)thiourea;
N-(2-chlorobenzyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2-ethyl-6-methylphenyl)thiourea;
N-(cis-4-{[4-(dimethylamino)quinazolin-2-yl]amino}cyclohexyl)-N′-(2-isopropylphenyl)thiourea;
N-(3,5-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(3,5-dimethylphenyl)thiourea;
N-(3-chloro-4-methylphenyl)-N′-(cis-4-{[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl}amino)benzoate;
N-(4-bromo-2,6-dimethylphenyl)-N′-(cis-4-{[4-(dimethylamino)quinazolin-2-yl]amino}cyclohexyl)thiourea;
N-(4-bromo-2-methylphenyl)-N′-(cis-4-{[4-(dimethylamino)quinazolin-2-yl]amino}-cyclohexyl)thiourea;
N-[4-bromo-2-(trifluoromethyl)phenyl]-N′-(cis-4-{[4-(dimethylamino)quinazolin-2-yl]amino}cyclohexyl)thiourea;
N-(4-chloro-2-methylphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-[1-(4-fluorophenyl)ethyl]thiourea;
N-(cis-4-{[4-(dimethylamino)quinazolin-2-yl]amino}cyclohexyl)-N′-(4-fluorobenzyl)thiourea;
N-(cis-4-{[4-(dimethylamino)quinazolin-2-yl]amino}cyclohexyl)-N′-(4-isopropylphenyl)thiourea;
N-(cis-4-{[4-(dimethylamino)quinazolin-2-yl]amino}cyclohexyl)-N′-(4-methoxybenzyl)thiourea;
methyl 4-({[(cis-4-{[4-(dimethylamino)quinazolin-2-yl]amino}cyclohexyl)amino]-carbonothioyl}amino)benzoate;
N-(cis-4-{[4-(dimethylamino)quinazolin-2-yl]amino}cyclohexyl)-N′-(1-phenylethyl)-thiourea;
N-(cis-4-{[4-(dimethylamino)quinazolin-2-yl]amino}cyclohexyl)-N′-(diphenylmethyl)thiourea;
N-(cyclohexylmethyl)-N′-(cis-4-{[4-(dimethylamino)quinazolin-2-yl]amino}cyclohexyl)thiourea;
N-cyclooctyl-N′-(cis-4-{[4-(dimethylamino)quinazolin-2-yl]amino}cyclohexyl)thiourea;
N-cyclopropyl-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(1-naphthylmethyl)thiourea;
N-(cis-4-{[4-(dimethylamino)quinazolin-2-yl]amino}cyclohexyl)-N′-(2,2-diphenylethyl)thiourea;
N-(2,3-dimethoxybenzyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2,4,5-trimethylphenyl)thiourea;
N-[2-(2,5-dimethoxyphenyl)ethyl]-N′-(cis-4-{[4-(dimethylamino)quinazolin-2-yl]amino}cyclohexyl)thiourea;
N-biphenyl-2-yl-N′-(cis-4-{[4-(dimethylamino)quinazolin-2-yl]amino}cyclohexyl)thiourea;
N-(cis-4-{[4-(dimethylamino)quinazolin-2-yl]amino})cyclohexyl)-N′-(2-fluorobenzyl)thiourea;
N-(cis-4-{[4-(dimethylamino)quinazolin-2-yl]amino}cyclohexyl)-N′-(2-methyl-4-nitrophenyl)thiourea;
N-(cis-4-{[4-(dimethylamino)quinazolin-2-yl]amino}cyclohexyl)-N′-(2-methylbenzyl)thiourea;
N-(3-chlorobenzyl)-N′-(cis-4-{[4-(dimethylamino)quinazolin-2-yl]amino}cyclohexyl)thiourea;
ethyl 3-({[(cis-4-{[4-(dimethylamino)quinazolin-2-yl]amino}cyclohexyl)amino]-carbonothioyl}amino)benzoate;
N-(cis-4-{[4-(dimethylamino)quinazolin-2-yl]amino}cyclohexyl)-N′-(3-ethylphenyl)-thiourea;
N-(cis-4-{[4-(dimethylamino)quinazolin-2-yl]amino}cyclohexyl)-N′-(3-fluorobenzyl)thiourea;
N-(cis-4-{[4-(dimethylamino)quinazolin-2-yl]amino}cyclohexyl)-N′-(3-methoxybenzyl)thiourea;
N-(cis-4-{[4-(dimethylamino)quinazolin-2-yl]amino}cyclohexyl)-N′-(3-methylbenzyl)thiourea;
N-[4-chloro-2-(trifluoromethyl)phenyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(4-fluoro-2-methylphenyl)thiourea;
N-(cis-4-{[4-(dimethylamino)quinazolin-2-yl]amino}cyclohexyl)-N′-(4-methoxy-2-methylphenyl)thiourea;
N-(5-chloro-2,4-dimethoxyphenyl)-N′-(cis-4-{[4-(dimethylamino)quinazolin-2-yl]amino}cyclohexyl)thiourea;
N-(2,3-dihydro-1 H-inden-5-yl)-N′-(cis-4-{[4-(dimethylamino)quinazolin-2-yl]amino}cyclohexyl)thiourea;
N-cycloheptyl-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[(1 R)-1-phenylethyl]thiourea;
N-(2-cyclohex-1-en-I -ylethyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2,3-dimethylphenyl)thiourea;
N-(2,4-dibromo-6-fluorophenyl)-N′-(cis-4-{[4-(dimethylamino)quinazolin-2-yl]amino}cyclohexyl)thiourea;
N-(2,4-dichloro-6-methylphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2,5-dimethylphenyl)thiourea;
N-(2-bromo-4-isopropylphenyl)-N′-(cis-4-{[4-(dimethylamino)quinazolin-2-yl]amino}cyclohexyl)thiourea;
N-(2-bromo-5-fluorophenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethoxyphenyl)thiourea;
N-(cis-4-{[4-(dimethylamino)quinazolin-2-yl]amino}cyclohexyl)-N′-(2-isopropyl-6-methylphenyl)thiourea;
N-(cis-4-{[4-(dimethylamino)quinazolin-2-yl]amino}cyclohexyl)-N′-(2-methoxybenzyl)thiourea;
N-(2,3-dihydro-1,4-benzodioxin-6-yl)-N′-(cis-4-{[4-(dimethylamino)quinazolin-2-yl]amino}cyclohexyl)thiourea;
N-1,3-benzodioxol-5-yl-N′-(cis-4-{[4-(dimethylamino)quinazolin-2-yl]amino}cyclohexyl)thiourea;
N-(3-chloro-2-methylphenyl)-N′-(cis-4-{[4-(dimethylamino)quinazolin-2-yl]amino}-cyclohexyl)thiourea;
N-[4-bromo-2-(trifluoromethoxy)phenyl]-N′-(cis-4-{[4-(dimethylamino)quinazolin-2-yl]amino}cyclohexyl)thiourea;
N-(4-chloro-2,5-dimethoxyphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(4-phenylbutyl)-thiourea;
N-bicyclo[2.2.1 ]hept-2-yl-N′-(cis-4-{[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl}amino)-4-methylthiophene-2-carboxylate;
methyl 3-({[(cis-4-{[4-(dimethylamino)quinazolin-2-yl]amino}cyclohexyl)amino]-carbonothioyl}amino)thiophene-2-carboxylate;
N-(2-bromo-4-fluorophenyl)-N′-(cis-4-{[4-(dimethylamino)quinazolin-2-yl]amino}-cyclohexyl)thiourea;
N-(4-butyl-2-methylphenyl)-N′-(cis-4-{[4-(dimethylamino)quinazolin-2-yl]amino}-cyclohexyl)thiourea;
N-[4-(dimethylamino)-1-naphthyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(5-methyl-3-phenylisoxazol-4-yl)thiourea;
N-[(cis-4-{[4-(dimethylamino)quinazolin-2-yl]amino}cyclohexyl)methyl]-N′-(2,6-dimethylphenyl)thiourea;
N-(2,6-dichlorophenyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2-ethyl-6-isopropylphenyl)thiourea;
N-[(cis-4-{[4-(dimethylamino)quinazolin-2-yl]amino}cyclohexyl)methyl]-N′-isobutylthiourea;
N-(1,3-benzodioxol-5-ylmethyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(4-nitrophenyl)thiourea;
N-[(cis-4-{[4-(dimethylamino)quinazolin-2-yl]amino}cyclohexyl)methyl]-N′-(pentafluorophenyl)thiourea;
N-[(cis-4-{[4-(dimethylamino)quinazolin-2-yl]amino}cyclohexyl)methyl]-N′-(tetrahydrofuran-2-ylmethyl)thiourea;
N-[(cis-4-{8 4-(dimethylamino)quinazolin-2-yl]amino}cyclohexyl)methyl]-N′-[2-(trifluoromethoxy)phenyl]thiourea;
N-[(cis-4-{[4-(dimethylamino)quinazolin-2-yl]amino}cyclohexyl)methyl]-N′-(2,3,4-trifluorophenyl)thiourea;
N-[(cis-4-{[4-(dimethylamino)quinazolin-2-yl]amino}cyclohexyl)methyl]-N′-(2-ethylphenyl)thiourea;
N-(5-chloro-2-methylphenyl)-N′-[(cis-4-{[4-(dimethylamino)quinazolin-2-yl]amino}-cyclohexyl)methyl]thiourea;
N-[(1 S,4R)-bicyclo[2.2.1 ]hept-2-yl]-N′-[(cis-4-{[4-(dimethylamino)quinazolin-2-yl]amino}cyclohexyl)methyl]thiourea;
N-[2-(3,4-dimethoxyphenyl)ethyl]-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2,4,6-tribromophenyl)thiourea;
N-[(cis-4-{[4-(dimethylamino)quinazolin-2-yl]amino}cyclohexyl)methyl]-N′-(2,4,6-trichlorophenyl)thiourea;
N-[(cis-4-{[4-(dimethylamino)quinazolin-2-yl]amino}cyclohexyl)methyl]-N′-mesitylthiourea;
N-(2,6-diethylphenyl)-N′-[(cis-4-{[4-(dimethylamino)quinazolin-2-yl]amino}cyclohexyl)methyl]thiourea;
N-(2,6-diisopropylphenyl)-N′-[(cis-4-{[4-(dimethylamino)quinazolin-2-yl]amino}-cyclohexyl)methyl]thiourea;
N-[(cis-4-{[4-(dimethylamino)quinazolin-2-yl]amino}cyclohexyl)methyl]-N′-(2-ethyl-6-methylphenyl)thiourea;
N-[(cis-4-{[4-(dimethylamino)quinazolin-2-yl]amino}cyclohexyl)methyl]-N′-(2-isopropylphenyl)thiourea;
N-(4-bromo-2,6-dimethylphenyl)-N′-[(cis-4-{[4-(dimethylamino)quinazolin-2-yl]amino}cyclohexyl)methyl]thiourea;
N-[4-bromo-2-(trifluoromethyl)phenyl]-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-[1-(4-fluorophenyl)ethyl]thiourea;
N-(5-chloro-2-methoxyphenyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(diphenylmethyl)thiourea;
N-cyclododecyl-N′-[(cis-4-{[4-(dimethylamino)quinazolin-2-yl]amino}cyclohexyl)methyl]thiourea;
N-(cyclohexylmethyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2,3,5,6-tetrachlorophenyl)thiourea;
N-(2,3-dimethoxybenzyl)-N′-[(cis-4-{[4-(dimethylamino)quinazolin-2-yl]amino}-cyclohexyl)methyl]thiourea;
N-(2,4-dichlorobenzyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2-methoxy-5-nitrophenyl)thiourea;
N-[(cis-4-{[4-(dimethylamino)quinazolin-2-yl]amino}cyclohexyl)methyl]-N′-(4-methoxy-2-methylphenyl)thiourea;
N-(2,4-dibromo-6-fluorophenyl)-N′-[(cis-4-{[4-(dimethylamino)quinazolin-2-yl]amino}cyclohexyl)methyl]thiourea;
N-(2,4-dichloro-6-methylphenyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2,5-dimethylphenyl)thiourea;
N-[(cis-4-{[4-(dimethylamino)quinazolin-2-yl]amino}cyclohexyl)methyl]-N′-(2-ethoxyphenyl)thiourea;
N-[(cis-4-{[4-(dimethylamino)quinazolin-2-yl]amino}cyclohexyl)methyl]-N′-(2-isopropyl-6-methylphenyl)thiourea;
N-[4-bromo-2-(trifluoromethoxy)phenyl]-N′-[(cis-4-{[4-(dimethylamino)quinazolin-2-yl]amino}cyclohexyl)methyl]thiourea;
N-bicyclo[2.2.1 ]hept-2-yl-N′-[(cis-4-{[4-(dimethylamino)quinazolin-2-yl]amino}-cyclohexyl)methyl]thiourea;
N-bicyclo[2.2.1 ]hept-5-en-2-yl-N′-[(cis-4-{[4-(dimethylamino)quinazolin-2-yl]amino}cyclohexyl)methyl]thiourea;
N-(cyclopropylmethyl)-N′-[(cis-4-{[4-(dimethylamino)quinazolin-2-yl]amino}cyclohexyl)methyl]thiourea; and
N-[(cis-4-{[4-(dimethylamino)quinazolin-2-yl]amino}cyclohexyl)methyl]-N′-(5-methyl-3-phenylisoxazol-4-yl)thiourea;
or a pharmaceutically acceptable salt, hydrate or solvate thereof.

15. The compound according to claim 1 selected from the group consisting of:
N-(4-bromophenyl)-N′-(cis-4-{[4-(dimethylamino)quinazolin-2-yl]amino}cyclohexyl)thiourea;
N-(4-cyanophenyl)-N′-(cis-4-{[4-(dimethylamino)quinazolin-2-yl]amino}cyclohexyl)thiourea;
N-(2,4-dichlorophenyl)-N′-(cis-4-{[4-(dimethylamino)quinazolin-2-yl]amino}cyclohexyl)thiourea;
N-(2,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2,6-dimethylphenyl)thiourea;
N-(cis-4-{[4-(dimethylamino)quinazolin-2-yl]amino}cyclohexyl)-N′-(2-ethyl-6-isopropylphenyl)thiourea;
N-(cis-4-{[4-(dimethylamino)quinazolin-2-yl]amino}cyclohexyl)-N′-(2-methoxyphenyl)thiourea;
N-(cis-4-{[4-(dimethylamino)quinazolin-2-yl]amino}cyclohexyl)-N′-1-naphthylthiourea;
N-(cis-4-{[4-(dimethylamino)quinazolin-2-yl]amino}cyclohexyl)-N′-(3,4,5-trimethoxyphenyl)thiourea;
N-(3,4-dimethoxyphenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(2-ethylphenyl)-thiourea;
N-(cis-4-{[4-(dimethylamino)quinazolin-2-yl]amino}cyclohexyl)-N′-(2-methoxy-4-nitrophenyl)thiourea;
N-(cis-4-{[4-(dimethylamino)quinazolin-2-yl]amino}cyclohexyl)-N′-(2-methoxy-5-methylphenyl)thiourea;
N-(4-bromo-2-chlorophenyl)-N′-(cis-4-{[4-(dimethylamino)quinazolin-2-yl]amino}-cyclohexyl)thiourea;
N-(cis-4-{[4-(dimethylamino)quinazolin-2-yl]amino}cyclohexyl)-N′-(4-iodophenyl)-thiourea;
N-(cis-4-{[4-(dimethylamino)quinazolin-2-yl]amino}cyclohexyl)-N′-(2,4,6-tribromophenyl)thiourea;
N-(cis-4-{[4-(dimethylamino)quinazolin-2-yl]amino}cyclohexyl)-N′-(2,4,6-trichlorophenyl)thiourea;
N-(cis-4-{[4-(dimethylamino)quinazolin-2-yl]amino}cyclohexyl)-N′-mesitylthiourea;
N-(cis-4-{[4-(dimethylamino)quinazolin-2-yl]amino}cyclohexyl)-N′-(2,4-dimethylphenyl)thiourea;
N-(2,6-diethylphenyl)-N′-(cis-4-{[4-(dimethylamino)quinazolin-2-yl]amino}cyclohexyl)thiourea;
N-(2-bromo-4-methylphenyl)-N′-(cis-4-{[4-(dimethylamino)quinazolin-2-yl]amino}-cyclohexyl)thiourea;
N-(2-chlorobenzyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2-ethyl-6-methylphenyl)thiourea;
N-(cis-4-{[4-(dimethylamino)quinazolin-2-yl]amino}cyclohexyl)-N′-(2-isopropylphenyl)thiourea;
methyl 3-({[(cis-4-{[4-(dimethylamino)quinazolin-2-yl]amino}cyclohexyl)amino]-carbonothioyl}amino)benzoate;
N-(4-bromo-2,6-dimethylphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(4-fluoro-2-methylphenyl)thiourea;
N-(cis-4-{[4-(dimethylamino)quinazolin-2-yl]amino}cyclohexyl)-N′-(4-methoxy-2-methylphenyl)thiourea;
N-(5-chloro-2,4-dimethoxyphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[(1R)-1-phenylethyl]thiourea;
N-(cis-4-{[4-(dimethylamino)quinazolin-2-yl]amino}cyclohexyl)-N′-(2,3-dimethylphenyl)thiourea;
N-(2,4-dibromo-6-fluorophenyl)-N′-(cis-4-{[4-(dimethylamino)quinazolin-2-yl]amino}cyclohexyl)thiourea;
N-(2,4-dichloro-6-methylphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2-ethoxyphenyl)thiourea;
N-(cis-4-{[4-(dimethylamino)quinazolin-2-yl]amino}cyclohexyl)-N′-(2-isopropyl-6-methylphenyl)thiourea;
N-(2,3-dihydro-1,4-benzodioxin-6-yl)-N′-(cis-4-{[4-(dimethylamino)quinazolin-2-yl]amino}cyclohexyl)thiourea;
N-1,3-benzodioxol-5-yl-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(4-fluoro-2-methylphenyl)thiourea; N-(cis-4-{[4-(dimethylamino)quinazolin-2-yl]amino}cyclohexyl)-N′-(4-methoxy-2-methylphenyl)thiourea;
N-(5-chloro-2,4-dimethoxyphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-[(1R)-1-phenylethyl]thiourea;
N-(cis-4-{[4-(dimethylamino)quinazolin-2-yl]amino}cyclohexyl)-N′-(2,3-dimethylphenyl)thiourea;
N-(2,4-dibromo-6-fluorophenyl)-N′-(cis-4-{[4-(dimethylamino)quinazolin-2-yl]amino}cyclohexyl)thiourea;
N-(2,4-dichloro-6-methylphenyl)-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(2-ethoxyphenyl)thiourea;
N-(cis-4-{[4-(dimethylamino)quinazolin-2-yl]amino}cyclohexyl)-N′-(2-isopropyl-6-methylphenyl)thiourea;
N-(2,3-dihydro-1,4-benzodioxin-6-yl)-N′-(cis-4-{[4-(dimethylamino)quinazolin-2-yl]amino}cyclohexyl)thiourea;
N-1,3-benzodioxol-5-yl-N′-(cis-4-{[4-(dimethylamino)quinazolin-2-yl]amino}cyclohexyl)thiourea;
N-(3-chloro-2-methylphenyl)-N′-(cis-4-{[4-(dimethylamino)quinazolin-2-yl]amino}-cyclohexyl)thiourea;
N-[4-bromo-2-(trifluoromethoxy)phenyl]-N′-(cis-4-{[4-(dimethylamino)quinazolin-2-yl]amino}cyclohexyl)thiourea;
N-(4-chloro-2,5-dimethoxyphenyl)-N′-(cis-4-{[4-(dimethylamino)quinazolin-2-yl]amino}cyclohexyl)thiourea;
N-bicyclo[2.2.1 ]hept-2-yl-N′-(cis-4-{[4-(dimethylamino)quinazolin-2-yl]amino}-cyclohexyl)thiourea;
methyl 3-({[(cis-4-{[4-(dimethylamino)quinazolin-2-yl]amino}cyclohexyl)amino]-carbonothioyl}amino)-4-methylthiophene-2-carboxylate;
methyl 3-({[(cis-4-{[4-(dimethylamino)quinazolin-2-yl]amino}cyclohexyl)amino]-carbonothioyl}amino)thiophene-2-carboxylate;
N-(4-butyl-2-methylphenyl)-N′-(cis-4-{[4-(dimethylamino)quinazolin-2-yl]amino}-cyclohexyl)thiourea;
N-[4-(dimethylamino)-1-naphthyl]-N′-(cis-4-{[4-(dimethylamino)quinazolin-2-yl]amino}cyclohexyl)thiourea;
N-(cis-4-{[4-(dimethylamino)quinazolin-2-yl]amino}cyclohexyl)-N′-(5-methyl-3-phenylisoxazol-4-yl)thiourea;
N-(2,6-diethylphenyl)-N′-[(cis-4-{[4-(dimethylamino)quinazolin-2-yl]amino}cyclohexyl)methyl]thiourea;
N-(4-bromo-2,6-dimethylphenyl)-N′-[(cis-4-{[4-(dimethylamino)quinazolin-2-yl]amino}cyclohexyl)methyl]thiourea;
N-[(cis-4-{[4-(dimethylamino)quinazolin-2-yl]amino}cyclohexyl)methyl]-N′-(2,3,5,6-tetrachlorophenyl)thiourea; and
N-[(cis-4-{[4-(dimethylamino)quinazolin-2-yl]amino}cyclohexyl)methyl]-N′-(2-isopropyl-6-methylphenyl)thiourea;
or a pharmaceutically acceptable salt, hydrate or solvate thereof.

16. The compound according to claim 3 wherein:
R₁ is selected from the group consisting of
(i) C₁₋₈ alkyl, and C₁₋₈ alkyl substituted by substituent(s) independently selected from the group consisting of:
halogen,
C₁₋₅ alkoxy,
C₁₋₅ alkoxy substituted by carbocyclic aryl,
carbocyclyl,
carbocyclic aryl,
carbocyclic aryl substituted by substituent(s) independently selected from the group consisting of:
halogen,
nitro, and
C₁₋₅ alkoxy,
(ii) C₂₋₅ alkenyl,
(iii) carbocyclyl,
(iv) carbocyclic aryl, and carbocyclic aryl substituted by substituent(s) independently selected from the group consisting of:
halogen,
C₁₋₅ alkyl,
C₁₋₅ alkyl substituted by halogen, and
C₁₋₅ alkoxy;
L is Formula (V); and
Y is —C(O)O—;
wherein carbocyclic aryl is phenyl or naphthyl;
carbocyclyl is 9H-fluorenyl or menthyl; and
halogen is fluoro, chloro, bromo, or iodo;
or a pharmaceutically acceptable salt, hydrate or solvate thereof.

17. The compound according to claim 16 wherein R₄ₐ is hydrogen or methyl; R_{4b} is methyl; R₅ and R₆ are hydrogen; A is a single bond; and B is a single bond or —CH₂—;
or a pharmaceutically acceptable salt, hydrate or solvate thereof.

18. The compound according to claim 1 selected from the group consisting of:
cis-[4-(4-dimethylamino-quinazolin-2-ylamino)-cyclohexyl]-carbamic acid 2-benzyloxy-ethyl ester;
cis-[4-(4-dimethylamino-quinazolin-2-ylamino)-cyclohexyl]-carbamic acid 4,5-dimethoxy-2-nitro-benzyl ester;
cis-[4-(4-dimethylamino-quinazolin-2-ylamino)-cyclohexyl]-carbamic acid 2-chloro-benzyl ester;
cis-[4-(4-dimethylamino-quinazolin-2-ylamino)-cyclohexyl]-carbamic acid 4,5-dimethoxy-2-nitro-benzyl ester;
cis-[4-(4-dimethylamino-quinazolin-2-ylamino)-cyclohexyl]-carbamic acid 4-nitro-benzyl ester;
cis-[4-(4-dimethylamino-quinazolin-2-ylamino)-cyclohexyl]-carbamic acid benzyl ester;
cis-[4-(4-dimethylamino-quinazolin-2-ylamino)-cyclohexylmethyl]-carbamic acid 2-chloro-benzyl ester;
cis-[4-(4-dimethylamino-quinazolin-2-ylamino)-cyclohexylmethyl]-carbamic acid 4-nitro-benzyl ester; and
cis-[4-(4-dimethylamino-quinazolin-2-ylamino)-cyclohexylmethyl]-carbamic acid benzyl ester;
or a pharmaceutically acceptable salt, hydrate or solvate thereof.

19. The compound according to claim 3 wherein:
R₁ is Cl-₈ alkyl, and
C₁₋₈ alkyl substituted by substituent(s) independently selected from the group consisting of:
carbocyclic aryl,
carbocyclic aryl substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxy substituted by halogen,
R₄ is —N(R₄ₐ)(R_{4b}) wherein R₄ₐ and R_{4b} are independently C₁₋₅ alkyl;
L is Formula (VIII) or (IX) wherein R₅ and R₆ are both hydrogen; A and B are each independently a single bond or —CH₂—; and
Y is a single bond;
wherein carbocyclic aryl is phenyl; and
halogen is fluoro or chloro;
or a pharmaceutically acceptable salt, hydrate or solvate thereof.

20. The compound according to claim 19 wherein:
R₁ is C₁₋₈ alkyl, and
C₁₋₈ alkyl substituted by substituent(s) independently selected from the group consisting of:
carbocyclic aryl,
carbocyclic aryl substituted by substituent(s) independently selected from the group consisting of:
C₁₋₅ alkoxy, and
C₁₋₅ alkoxy substituted by halogen,
wherein carbocyclic aryl is phenyl; and
halogen is fluoro or chloro;
or a pharmaceutically acceptable salt, hydrate or solvate thereof.

21. The compound according to claim 20 wherein:
R₄ is —N(CH₃)₂; L is Formula (VIII) or (IX) wherein A is a single bond and B is —CH₂—, or A is —CH₂— and B is a single bond; and Y is a single bond;
wherein carbocyclic aryl is phenyl; and
halogen is fluoro;
or a pharmaceutically acceptable salt, hydrate or solvate thereof.

22. The compound according to claim 1 is:
N2-[(1S,3R)-3-({[4-bromo-2-(trifluoromethoxy)benzyl]amino}-methyl)cyclopentyl]-N4,N4-dimethylquinazoline-2,4-diamine;
or a pharmaceutically acceptable salt, hydrate or solvate thereof.

23. The compound according to claim 3 wherein:
R₁ is selected from the group consisting of:
(i) C₁₋₈ alkyl, and
C₁₋₈ alkyl substituted by substituent(s) independently selected from the group consisting of:
carbocyclic aryl,
carbocyclic aryl substituted by substituent(s) independently selected from the group consisting of:
hydroxy,
halogen,
nitro,
C₁₋₅ alkylcarbonylamino,
C₃₋₆ cycloalkylcarbonylamino,
C₁₋₅ alkyl,
C₁₋₅ alkyl substituted by halogen,
C₁₋₅ alkylsulfonyl,
C₁₋₅ alkoxy,
C₁₋₅ alkoxy substituted by halogen, and
carbocyclic aryl,
heterocyclyl, and
heterocyclyl substituted by halogen,
(ii) C₃₋₁₂ cycloalkyl, and C₃-1₂ cycloalkyl substituted by carbocyclic aryl,
(iii) carbocyclyl, and carbocyclyl by substituent(s) independently selected from the group consisting of:
hydroxy, and
carbocyclic aryl,
(iv) carbocyclic aryl, and carbocyclic aryl substituted by substituent(s) independently selected from the group consisting of:
halogen,
C₁₋₅ alkoxy, and
nitro,
(v) heterocyclyl, and heterocyclyl substituted by substituent(s) independently selected from the group consisting of:
halogen, and
C₁₋₅ alkoxy,
R₄ is —N(R₄ₐ)(R_{4b}) wherein R₄ₐ and R_{4b} are each independently C₁₋₅ alkyl;
L is Formula (XIII); wherein R₅ and R₆ are both hydrogen; A is a single bond and B is a single bond or —CH₂—; and
Y is —C(O)NR₇—, wherein R₇ is hydrogen or C₁₋₅ alkyl;
wherein carbocyclic aryl is phenyl or naphthyl;
carbocyclyl is indanyl, 9H-fluorenyl, 1,2,3,4-tetrahydro-naphthalen-1-yl, or 1H-indolyl;
heterocyclyl is benzo[1,3]dioxolyl, pyridyl, dibenzofuranyl, 1H-benzoimidazolyl, or thiazolyl; and
halogen is fluoro, chloro, bromo, or iodo;
or a pharmaceutically acceptable salt, hydrate or solvate thereof.

24. The compound according to claim 23 wherein:
R₁ is selected from the group consisting of:
(i) C₁₋₈ alkyl, and C₁₋₈ alkyl substituted by substituent(s) independently selected from the group consisting of:
carbocyclic aryl,
carbocyclic aryl substituted by substituent(s) independently selected from the group consisting of:
hydroxy,
halogen,
nitro,
C₁₋₅ alkylcarbonylamino,
C₁₋₅ alkyl,
C₁₋₅ alkyl substituted by halogen,
C₁₋₅ alkylsulfonyl,
C₁₋₅ alkoxy,
₁₋₅ alkoxy substituted by halogen, and
carbocyclic aryl,
heterocyclyl, and
heterocyclyl substituted by halogen,
(ii) C₃₋₁₂ cycloalkyl, and
C₃-1₂ cycloalkyl substituted by carbocyclic aryl,
(iii) carbocyclyl,
(iv) carbocyclic aryl, and carbocyclic aryl substituted by substituent(s) independently selected from the group consisting of:
halogen, and
nitro,
(v) heterocyclyl, and heterocyclyl substituted by substituent(s) independently selected from the group consisting of:
halogen, and
C₁₋₅ alkoxy,
wherein carbocyclic aryl is phenyl or naphthyl;
carbocyclyl is indanyl, 9H-fluorenyl, or 1,2,3,4-tetrahydro-naphthalen-1-yl;
heterocyclyl is benzo[1,3]dioxolyl, or pyridyl; and
halogen is fluoro, chloro, bromo, or iodo;
or a pharmaceutically acceptable salt, hydrate or solvate thereof.

25. The compound according to claim 24 wherein R₄ is —N(CH₃)₂; A and B are both a single bond; and Y is —C(O)NH—;
or a pharmaceutically acceptable salt, hydrate or solvate thereof.

26. The compound according to claim 1 selected from the group consisting of:
cis-4-{[4-(dimethylamino)quinazolin-2-yl]amino}-N-(2,3-dimethylbenzyl)-cyclohexanecarboxamide;
cis-N-(2-bromobenzyl)-4-{[4-(dimethylamino)quinazolin-2-yl]amino}-cyclohexanecarboxamide;
cis-N-(2-chlorobenzyl)-4-{[4-(dimethylamino)quinazolin-2-yl]amino}-cyclohexanecarboxamide;
cis-4-{[4-(dimethylamino)quinazolin-2-yl]amino}-N-(4-methylbenzyl)-cyclohexanecarboxamide;
cis-N-(3,5-bis(trifluoromethyl)benzyl)-4-{[4-(dimethylamino)quinazolin-2-yl]amino}cyclohexanecarboxamide;
cis-N-(2,4-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-(1,2,3,4-tetrahydronaphthalen-1-yl)cyclohexanecarboxamide;
cis-N-(2,3-dihydro-1H-inden-2-yl)-4-{[4-(dimethylamino)quinazolin-2-yl]amino}-cyclohexanecarboxamide;
cis-4-{[4-(dimethylamino)quinazolin-2-yl]amino}-N-[(1S)-i -(4-nitrophenyl)ethyl]-cyclohexanecarboxamide;
cis-N-(3,5-dichlorobenzyl)-4-{[4-(dimethylamino)quinazolin-2-yl]amino}-cyclohexanecarboxamide;
cis-4-{[4-(dimethylamino)quinazolin-2-yl]amino}-N-[4-(trifluoromethoxy)benzyl]-cyclohexanecarboxamide;
cis-N-(4-bromobenzyl)-4-{[4-(dimethylamino)quinazolin-2-yl]amino}-cyclohexanecarboxamide;
cis-4-{[4-(dimethylamino)quinazolin-2-yl]amino}-N-(4-methoxybenzyl)-cyclohexanecarboxamide;
cis-4-{[4-(dimethylamino)quinazolin-2-yl]amino}-N-(2-fluoro-4-nitrophenyl)-cyclohexanecarboxamide;
cis-4-{[4-(dimethylamino)quinazolin-2-yl]amino}-N-(3-fluoro-4-methylbenzyl)-cyclohexanecarboxamide;
cis-N-(5-chloro-2-methylbenzyl)-4-{[4-(dimethylamino)quinazolin-2-yl]amino}-cyclohexanecarboxamide; and
cis-N-(2,4-dichloro-6-methylbenzyl)-4-{[4-(dimethylamino)quinazolin-2-yl]amino}-cyclohexanecarboxamide;
or a pharmaceutically acceptable salt, hydrate or solvate thereof.

27. The compound according to claim 1 selected form the group consisting of:
cis-N-(2,3-dimethoxybenzyl)-4-{[4-(dimethylamino)quinazolin-2-yl]amino}-cyclohexanecarboxamide;
cis-N-(2,4-difluorobenzyl)-4-{[4-(dimethylamino)quinazolin-2-yl]amino}-cyclohexanecarboxamide;
cis-N-(2,4-dichlorobenzyl)-4-{[4-(dimethylamino)quinazolin-2-yl]amino}-cyclohexanecarboxamide;
cis-N-(2,3-dichlorobenzyl)-4-{[4-(dimethylamino)quinazolin-2-yl]amino}-cyclohexanecarboxamide;
cis-N-(2,5-dichlorobenzyl)-4-{[4-(dimethylamino)quinazolin-2-yl]amino}-cyclohexanecarboxamide;
cis-N-(3-chlorobenzyl)-4-{[4-(dimethylamino)quinazolin-2-yl]amino}-cyclohexanecarboxamide;
cis-4-{[4-(dimethylamino)quinazolin-2-yl]amino}-N-(3-methoxybenzyl)-cyclohexanecarboxamide;
cis-N-(3,4-dimethoxybenzyl)-4-{[4-(dimethylamino)quinazolin-2-yl]amino}-cyclohexanecarboxamide;
cis-N-(3,5-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-(4-hydroxy-3-methoxybenzyl)-cyclohexanecarboxamide;
cis-N-(1,3-benzodioxol-5-ylmethyl)-4-{[4-(dimethylamino)quinazolin-2-yl]amino}-cyclohexanecarboxamide;
cis-4-{[4-(dimethylamino)quinazolin-2-yl]amino}-N-[(1R)-1-(4-nitrophenyl)ethyl]-cyclohexanecarboxamide;
cis-4-(4-dimethylamino-quinazolin-2-ylamino)-cyclohexanecarboxylic acid (trans-2-phenylcyclopropyl)amide;
cis-4-{[4-(dimethylamino)quinazolin-2-yl]amino}-N-[(l S)-1-(4-methylphenyl)ethyl]-cyclohexanecarboxamide;
cis-4-{[4-(dimethylamino)quinazolin-2-yl]amino}-N-[(l R)-1-(1-naphthyl)ethyl]-cyclohexanecarboxamide;
cis-4-{[4-(dimethylamino)quinazolin-2-yl]amino}-N-[3-(trifluoromethyl)benzyl]-cyclohexanecarboxamide;
cis-4-{[4-(dimethylamino)quinazolin-2-yl]amino}-N-(3-methoxyphenyl)-cyclohexanecarboxamide;
cis-4-{[4-(dimethylamino)quinazolin-2-yl]amino}-N-(3-iodobenzyl)-cyclohexanecarboxamide;
cis-4-{[4-(dimethylamino)quinazolin-2-yl]amino}-N-(4-methoxybenzyl)-cyclohexanecarboxamide;
cis-4-{[4-(dimethylamino)quinazolin-2-yl]amino}-N-(3-iodophenyl)-cyclohexanecarboxamide;
cis-4-{[4-(dimethylamino)quinazolin-2-yl]amino}-N-[3-(propionylamino)benzyl]-cyclohexanecarboxamide;
cis-N-benzyl-4-{[4-(dimethylamino)quinazolin-2-yl]amino}-cyclohexanecarboxamide;
cis-N-[(6-chloropyridin-3-yl)methyl]-4-{[4-(dimethylamino)quinazolin-2-yl]amino}-cyclohexanecarboxamide;
cis-4-{[4-(dimethylamino)quinazolin-2-yl]amino}-N-[(1R)-1-(3-methoxyphenyl)ethyl]cyclohexanecarboxamide;
cis-4-{[4-(dimethylamino)quinazolin-2-yl]amino}-N-[1-(4-fluorophenyl)ethyl]-cyclohexanecarboxamide;
cis-N-[(1R)-1-(4-chlorophenyl)ethyl]-4-{[4-(dimethylamino)quinazolin-2-yl]amino}-cyclohexanecarboxamide;
cis-N-[1-(4-bromophenyl)ethyl]-4-{[4-(dimethylamino)quinazolin-2-yl]amino}-cyclohexanecarboxamide;
cis-4-{[4-(dimethylamino)quinazolin-2-yl]amino}-N-[(1 S)-I -(1-naphthyl)ethyl]-cyclohexanecarboxamide;
cis-4-{[4-(dimethylamino)quinazolin-2-yl]amino}-N-(3,5-dimethylbenzyl)-cyclohexanecarboxamide;
cis-N-(3-chloro-2-methylbenzyl)-4-{[4-(dimethylamino)quinazolin-2-yl]amino}-cyclohexanecarboxamide;
cis-4-{[4-(dimethylamino)quinazolin-2-yl]amino}-N-(5-fluoro-2-methylbenzyl)-cyclohexanecarboxamide;
cis-N-(3-chloro-2,6-difluorobenzyl)-4-{[4-(dimethylamino)quinazolin-2-yl]amino}-cyclohexanecarboxamide;
cis-N-(biphenyl-3-ylmethyl)-4-{[4-(dimethylamino)quinazolin-2-yl]amino}-cyclohexanecarboxamide;
cis-N-(biphenyl-4-ylmethyl)-4-{[4-(dimethylamino)quinazolin-2-yl]amino}-cyclohexanecarboxamide;
cis-N-(6-chloro-2-fluoro-3-methylbenzyl)-4-{[4-(dimethylamino)quinazolin-2-yl]amino}cyclohexanecarboxamide;
cis-4-{[4-(dimethylamino)quinazolin-2-yl]amino}-N-(2-fluorobenzyl)-cyclohexanecarboxamide;
cis-N-(2,6-difluorobenzyl)-4-{[4-(dimethylamino)quinazolin-2-yl]amino}-cyclohexanecarboxamide;
cis-4-{[4-(dimethylamino)quinazolin-2-yl]amino}-N-[4-(trifluoromethyl)benzyl]-cyclohexanecarboxamide;
cis-4-{[4-(dimethylamino)quinazolin-2-yl]amino}-N-(1-naphthylmethyl)-cyclohexanecarboxamide;
cis-N-(4-chlorobenzyl)-4-{[4-(dimethylamino)quinazolin-2-yl]amino}-cyclohexanecarboxamide;
cis-N-(3,4-dichlorobenzyl)-4-{[4-(dimethylamino)quinazolin-2-yl]amino}-cyclohexanecarboxamide;
cis-4-{[4-(dimethylamino)quinazolin-2-yl]amino}-N-(3-fluorobenzyl)-cyclohexanecarboxamide;
cis-N-(2,5-difluorobenzyl)-4-{[4-(dimethylamino)quinazolin-2-yl]amino}-cyclohexanecarboxamide;
cis-N-(2,3-difluorobenzyl)-4-{[4-(dimethylamino)quinazolin-2-yl]amino}-cyclohexanecarboxamide;
cis-N-(3-bromobenzyl)-4-{[4-(dimethylamino)quinazolin-2-yl]amino}-cyclohexanecarboxamide;
cis-N-(3-bromo-4-fluorobenzyl)-4-{[4-(dimethylamino)quinazolin-2-yl]amino}-cyclohexanecarboxamide;
cis-N-(4-bromo-2-fluorobenzyl)-4-{[4-(dimethylamino)quinazolin-2-yl]amino}-cyclohexanecarboxamide;
cis-N-(5-bromo-2-fluorobenzyl)-4-{[4-(dimethylamino)quinazolin-2-yl]amino}-cyclohexanecarboxamide;
cis-N-(4-chloro-2-fluorobenzyl)-4-{[4-(dimethylamino)quinazolin-2-yl]amino}-cyclohexanecarboxamide;
cis-4-{[4-(dimethylamino)quinazolin-2-yl]amino}-N-(3-methylbenzyl)-cyclohexanecarboxamide;
cis-4-{[4-(dimethylamino)quinazolin-2-yl]amino}-N-(2-methylbenzyl)-cyclohexanecarboxamide;
cis-4-{[4-(dimethylamino)quinazolin-2-yl]amino}-N-[2-(trifluoromethoxy)benzyl]-cyclohexanecarboxamide;
cis-4-{[4-(dimethylamino)quinazolin-2-yl]amino}-N-(2,3,4-trifluorobenzyl)-cyclohexanecarboxamide;
cis-4-{[4-(dimethylamino)quinazolin-2-yl]amino}-N-(2,4,5-trifluorobenzyl)-cyclohexanecarboxamide;
cis-4-{[4-(dimethylamino)quinazolin-2-yl]amino}-N-(3,4,5-trifluorobenzyl)-cyclohexanecarboxamide;
cis-4-{[4-(dimethylamino)quinazolin-2-yl]amino}-N-(2,3,6-trifluorobenzyl)-cyclohexanecarboxamide;
cis-4-{[4-(dimethylamino)quinazolin-2-yl]amino}-N-[3-fluoro-5-(trifluoromethyl)benzyl]cyclohexanecarboxamide;
cis-4-{[4-(dimethylamino)quinazolin-2-yl]amino}-N-[4-fluoro-2-(trifluoromethyl)benzyl]cyclohexanecarboxamide;
cis-4-{[4-(dimethylamino)quinazolin-2-yl]amino}-N-[2-fluoro-4-(trifluoromethyl)benzyl]cyclohexanecarboxamide;
cis-4-{[4-(dimethylamino)quinazolin-2-yl]amino}-N-[4-fluoro-3-(trifluoromethyl)benzyl]cyclohexanecarboxamide;
cis-4-{[4-(dimethylamino)quinazolin-2-yl]amino}-N-[2-fluoro-3-(trifluoromethyl)benzyl]cyclohexanecarboxamide;
cis-N-[4-chloro-3-(trifluoromethyl)benzyl]-4-{[4-(dimethylamino)quinazolin-2-yl]amino}cyclohexanecarboxamide;
cis-N-(2-chloro-6-fluorobenzyl)-4-{[4-(dimethylamino)quinazolin-2-yl]amino}-cyclohexanecarboxamide;
cis-N-(3-chloro-4-fluorobenzyl)-4-{[4-(dim ethyl amino)quinazolin-2-yl]amino}-cyclohexanecarboxamide;
cis-N-(2-chloro-4-fluorobenzyl)-4-{[4-(dimethylamino)quinazolin-2-yl]amino}-cyclohexanecarboxamide;
cis-N-[2-chloro-5-(trifluoromethyl)benzyl]-4-{[4-(dimethylamino)quinazolin-2-yl]amino}cyclohexanecarboxamide;
cis-N-[2-(difluoromethoxy)benzyl]-4-{[4-(dimethylamino)quinazolin-2-yl]amino}-cyclohexanecarboxamide;
cis-N-[3-(difluoromethoxy)benzyl]-4-{[4-(dimethylamino)quinazolin-2-yl]amino}-cyclohexanecarboxamide;
cis-4-{[4-(dimethylamino)quinazolin-2-yl]amino}-N-[3-(trifluoromethoxy)benzyl]-cyclohexanecarboxamide;
cis-N-(2,6-dimethoxybenzyl)-4-{[4-(dimethylamino)quinazolin-2-yl]amino}-cyclohexanecarboxamide;
cis-4-{[4-(dimethylamino)quinazolin-2-yl]amino}-N-[(1R)-1-phenylethyl]-cyclohexanecarboxamide;
cis-4-{[4-(dimethylamino)quinazolin-2-yl]amino}-N-[(1S)-1-(4-methoxyphenyl)ethyl]cyclohexanecarboxamide;
cis-N-[bis(4-methoxyphenyl)methyl]-4-{[4-(dimethylamino)quinazolin-2-yl]amino}-cyclohexanecarboxamide;
cis-4-{[4-(dimethylamino)quinazolin-2-yl]amino}-N-[2-(trifluoromethyl)benzyl]-cyclohexanecarboxamide;
cis-4-{[4-(dimethylamino)quinazolin-2-yl]amino}-N-9H-fluoren-9-ylcyclohexanecarboxamide;
cis-4-{[4-(dimethylamino)quinazolin-2-yl]amino}-N-[4-(methylsulfonyl)benzyl]-cyclohexanecarboxamide; and
cis-N-(6-chloropyridin-3-yl)-4-{[4-(dimethylamino)quinazolin-2-yl]amino}- cyclohexanecarboxamide;
or a pharmaceutically acceptable salt, hydrate or solvate thereof.

28. The compound according to claim 3 wherein:
R₁ is selected from the group consisting of:
(i) C₁₋₈ alkyl, and C₁₋₈ alkyl substituted by substituent(s) independently selected from the group consisting of:
carbocyclic aryl,
carbocyclic aryl substituted by substituent(s) independently selected from the group consisting of:
C₁₋₅ alkoxy, and
C₁₋₅ alkoxy substituted by halogen,
(ii) carbocyclic aryl, and carbocyclic aryl substituted by substituent(s) independently selected from the group consisting of:
halogen, and
C₁₋₇ alkoxy,
R₄ is —N(R₄ₐ)(R_{4b}) wherein R₄ₐ and R_{4b} are each independently C₁₋₅ alkyl;
L is Formula (XIII) wherein R₅ is hydrogen; A is a single bond and B is a single bond or —CH₂—; and
Y is —C(O)O— or —OC(O)—;
wherein carbocyclic aryl is phenyl or naphthyl; and
halogen is fluoro, chloro, bromo, or iodo;
or a pharmaceutically acceptable salt, hydrate or solvate thereof.

29. The compound according to claim 28 wherein R₄ is —N(CH₃)₂;
or a pharmaceutically acceptable salt, hydrate or solvate thereof.

30. The compound according to claim 3 wherein:
R₁ is selected from the group consisting of:
carbocyclic aryl, and
carbocyclic aryl substituted by substituent(s) independently selected from the group consisting of:
halogen,
C₁₋₁₀ alkyl,
C₁₋₁₀ alkyl substituted by halogen,
C₁₋₇ alkoxy, and
C₁₋₇ alkoxy substituted by halogen,
R₄ is —N(R₄ₐ)(R_{4b}) wherein R₄ₐ and R_{4b} are each independently C₁₋₅ alkyl;
L is Formula (VIII) or (IX) wherein A and B are each independently a single bond or
—CH₂—; and
Y is —C(O)—,
wherein carbocyclic aryl is phenyl; and
halogen is fluoro, chloro, bromo, or iodo;
or a pharmaceutically acceptable salt, hydrate or solvate thereof.

31. The compound according to claim 30 wherein R₄ is —N(CH₃)₂; R₅ and R₆ are both hydrogen; and A is a single bond, and B is —CH₂—; or A is a —CH₂—, and B is a single bond,
or a pharmaceutically acceptable salt, hydrate or solvate thereof.

32. The compound according to claim 1 selected from the group consisting of: p1 3,4-dichloro-N-[((1R,3S)-3-{[4-(dimethylamino)quinazolin-2-yl]amino}cyclopentyl)-methyl]benzamide;
N-[(1 S,3R)-3-({[4-(dimethylamino)quinazolin-2-yl]amino}methyl)cyclopentyl]-4-fluorobenzamide;
4-chloro-N-[((1R,3S)-3-{[4-(dimethylamino)quinazolin-2-yl]amino}cyclopentyl)methyl]benzamide; and
N-[((1 R,3 S)-3-{[4-(dimethylamino)quinazolin-2-yl]amino}cyclopentyl)methyl]-3,5-difluorobenzamide;
or a pharmaceutically acceptable salt, hydrate or solvate thereof.

33. The compound according to claim 1 selected from the group consisting of:
N-[((1R,3S)-3-{[4-(dimethylamino)quinazolin-2-yl]amino}cyclopentyl)methyl]-3,5-dimethoxybenzamide;
2,4,6-trichloro-N-[((1R,3S)-3-{[4-(dimethylamino)quinazolin-2-yl]amino}cyclopentyl)methyl]benzamide;
N-[((1R,3S)-3-{[4-(dimethylamino)quinazolin-2-yl]amino}cyclopentyl)methyl]-3-fluoro-4-(trifluoromethyl)benzamide;
N-[((1R,3S)-3-{[4-(dimethylamino)quinazolin-2-yl]amino}cyclopentyl)methyl]-4-(trifluoromethoxy)benzamide; and
N-[(1S,3R)-3-({[4-(dimethylamino)quinazolin-2-yl]amino}methyl)cyclopentyl]-2,4-difluorobenzamide;
or a pharmaceutically acceptable salt, hydrate or solvate thereof.

34. The compound according to claim 2 wherein Q is Formula (IIa).

35. The compound according to claim 34 wherein:
R₁ is selected from the group consisting of:
(i) C₁₋₈ alkyl, and C₁₋₈ alkyl substituted by carbocyclic aryl,
(ii) carbocyclic aryl, and carbocyclic aryl substituted by substituent(s) independently selected from the group consisting of:
halogen,
C₁₋₁₀ alkyl,
C₁₋₁₀ alkyl substituted by halogen,
C₁₋₇ alkoxy, and
C₁₋₇ alkoxy substituted by halogen,
R₂ is —N(R₂ₐ)(R_{2b}), wherein R₂ₐ and R_{2b} are each independently C₁₋₅ alkyl;
L is Formula (V) wherein R₅ and R₆ are both hydrogen; A and B are both a single bond;
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is —C(O)—;
wherein carbocyclic aryl is phenyl; and
halogen is fluoro, chloro, bromo, or iodo;
or a pharmaceutically acceptable salt, hydrate or solvate thereof.

36. The compound according to claim 35 wherein R₂ is —N(CH₃)₂; and X₁, X₂, X₃ and X₄ are independently selected from the group consisting of hydrogen, fluoro, and methyl; provided that at least one substituent selected from the group consisting of X₁, X₂, X₃ and X₄ is not hydrogen;
or a pharmaceutically acceptable salt, hydrate or solvate thereof.

37. The compound according to claim 1 selected from the group consisting of:
N-(cis-4-{[4-(dimethylamino)-6-methylquinazolin-2-yl]amino}cyclohexyl)-2,2-diphenylacetamide;
N-(cis-4-{[4-(dimethylamino)-6-methylquinazolin-2-yl]amino}cyclohexyl)-4-fluoro-3-(trifluoromethyl)benzamide;
N-(cis-4-{[4-(dimethylamino)-6-methylquinazolin-2-yl]amino}cyclohexyl)-3,5-bis(trifluoromethyl)benzamide; and
N-(cis-4-{[4-(dimethylamino)-6-methylquinazolin-2-yl]amino}cyclohexyl)-3,4,5-trimethoxybenzamide;
or a pharmaceutically acceptable salt, hydrate or solvate thereof.

38. The compound according to claim 1 selected from the group consisting of:
3-chloro-N-(cis-4-{[4-(dimethylamino)-6,7-difluoroquinazolin-2-yl]amino}cyclohexyl)benzamide;
3,4-dichloro-N-(cis-4-{[4-(dimethylamino)-6,7-difluoroquinazolin-2-yl]amino}cyclohexyl)benzamide;
N-(cis-4-{[4-(dimethylamino)-6,7-difluoroquinazolin-2-yl]amino}cyclohexyl)-3,5-dimethoxybenzamide;
N-(cis-4-{[4-(dimethylamino)-6,7-difluoroquinazolin-2-yl]amino}cyclohexyl)benzamide;
N-(cis-4-{[4-(dimethylamino)-6,7-difluoroquinazolin-2-yl]amino}cyclohexyl)-4-methylbenzamide;
N-(cis-4-{[4-(dimethylamino)-6,7-difluoroquinazolin-2-yl]amino}cyclohexyl)-4-fluorobenzamide;
N-(cis-4-{[4-(dimethylamino)-6,7-difluoroquinazolin-2-yl]amino}cyclohexyl)-3-methoxybenzamide;
N-(cis-4-{[4-(dimethylamino)-6,7-difluoroquinazolin-2-yl]amino}cyclohexyl)-3,4-difluorobenzamide; and
N-(cis-4-{[4-(dimethylamino)-6,7-difluoroquinazolin-2-yl]amino}cyclohexyl)-3-(trifluoromethyl)benzamide;
or a pharmaceutically acceptable salt, hydrate or solvate thereof.

39. The compound according to claim 34 wherein:
R₁ is selected from the group consisting of:
(i) C₁₋₈ alkyl, and C₁₋₈ alkyl substituted by substituent(s) independently selected from the group consisting of:
carbocyclic aryl,
carbocyclic aryl substituted by substituent(s) independently selected from the group consisting of:
halogen,
C₁₋₅ alkyl,
C₁₋₅ alkyl substituted by halogen,
C₁₋₅ alkoxy, and
C₁₋₅ alkoxy substituted by halogen,
(ii) heterocyclyl, and heterocyclyl substituted by halogen,
R₂ is —N(R₂ₐ)(R_{2b}), wherein R₂ₐ and R_{2b} are each independently C₁₋₅ alkyl;
L is Formula (XIII);
X₁, X₂, X₃ and X₄ are independently hydrogen or halogen; provided that at least one substituent selected from the group consisting of X₁, X₂, X₃ and X₄ is not hydrogen; and
Y is —C(O)NR₇— wherein R₇ is hydrogen or C₁₋₅ alkyl;
wherein carbocyclic aryl is phenyl;
heterocyclyl is pyridyl; and
halogen is fluoro, chloro, bromo, or iodo;
or a pharmaceutically acceptable salt, hydrate or solvate thereof.

40. The compound according to claim 39 wherein R₂ is —N(CH₃)₂; L is Formula (XIII) wherein A and B are both a single bond; X₁, X₂, X₃ and X₄ are independently hydrogen or fluoro; provided that at least one substituent selected from the group consisting of X₁, X₂, X₃ and X₄ is not hydrogen; and Y is —C(O)NH—;
or a pharmaceutically acceptable salt, hydrate or solvate thereof.

41. The compound according to claim 1 selected from the group consisting of:
cis-N-benzyl-4-{[4-(dimethylamino)-6,7-difluoroquinazolin-2-yl]amino}-cyclohexanecarboxamide;
cis-N-(3,5-dimethoxybenzyl)-4-{[4-(dimethylamino)-6,7-difluoroquinazolin-2-yl]amino}cyclohexanecarboxamide;
cis-4-{[4-(dimethylamino)-6,7-difluoroquinazolin-2-yl]amino}-N-(3-methoxybenzyl)cyclohexanecarboxamide;
cis-N-[(6-chloropyridin-3-yl)methyl]-4-{[4-(dimethylamino)-6,7-difluoroquinazolin-2-yl]amino}cyclohexanecarboxamide;
cis-4-{[4-(dimethylamino)-6,7-difluoroquinazolin-2-yl]amino}-N-[3-(trifluoromethyl)benzyl]cyclohexanecarboxamide;
cis-4-{[4-(dimethylamino)-6,7-difluoroquinazolin-2-yl]amino}-N-[4-(trifluoromethyl)benzyl]cyclohexanecarboxamide;
cis-N-[3,5-bis(trifluoromethyl)benzyl]-4-{[4-(dimethylamino)-6,7-difluoroquinazolin-2-yl]amino}cyclohexanecarboxamide;
cis-4-{[4-(dimethylamino)-6,7-difluoroquinazolin-2-yl]amino}-N-(3-iodobenzyl)-cyclohexanecarboxamide; and
cis-N-[1-(4-bromophenyl)ethyl]-4-{[4-(dimethylamino)-6,7-difluoroquinazolin-2-yl]amino}cyclohexanecarboxamide;
or a pharmaceutically acceptable salt, hydrate or solvate thereof.

42. The compound according to claim 1 selected from the group consisting of:
cis-4-{[4-(dimethylamino)-6,7-difluoroquinazolin-2-yl]amino}-N-(4-methylbenzyl)-cyclohexanecarboxamide;
cis-N-(3-chlorobenzyl)-4-{[4-(dimethylamino)-6,7-difluoroquinazolin-2-yl]amino}-cyclohexanecarboxamide;
cis-4-{[4-(dimethylamino)-6,7-difluoroquinazolin-2-yl]amino}-N-[(1R)-1-(3-methoxyphenyl)ethyl]cyclohexanecarboxamide;
cis-4-{[4-(dimethylamino)-6,7-difluoroquinazolin-2-yl]amino}-N-(4-methoxybenzyl)cyclohexanecarboxamide;
cis-N-(2,4-dichlorobenzyl)-4-{[4-(dimethylamino)-6,7-difluoroquinazolin-2-yl]amino}cyclohexanecarboxamide;
cis-N-(3,5-dichlorobenzyl)-4-{[4-(dimethylamino)-6,7-difluoroquinazolin-2-yl]amino}cyclohexanecarboxamide;
cis-N-(4-bromobenzyl)-4-{[4-(dimethylamino)-6,7-difluoroquinazolin-2-yl]amino}-cyclohexanecarboxamide;
cis-N-(2-bromobenzyl)-4-{[4-(dimethylamino)-6,7-difluoroquinazolin-2-yl]amino}-cyclohexanecarboxamide;
cis-4-{[4-(dimethylamino)-6,7-difluoroquinazolin-2-yl]amino}-N-[4-(trifluoromethoxy)benzyl]cyclohexanecarboxamide; and
cis-4-{[4-(dimethylamino)-6,7-difluoroquinazolin-2-yl]amino}-N-[(1S)-1-(4-methylphenyl)ethyl]cyclohexanecarboxamide;
or a pharmaceutically acceptable salt, hydrate or solvate thereof.

43. The compound according to claim 2 wherein Q is Formula (IIb).

44. The compound according to claim 43 wherein:
R₁ is selected from the group consisting of:
C₁₋₈ alkyl, and
C₁₋₈ alkyl substituted by substituent(s) independently selected from the group consisting of:
carbocyclic aryl,
carbocyclic aryl substituted by substituent(s) independently selected from the group consisting of:
halogen,
C₁₋₅ alkyl, and
C₁₋₅ alkoxy,
R₃ is C₁₋₅ alkyl;
L is Formula (XIII); wherein R₅ and R₆ are both hydrogen; A and B are both a single bond;
Y is —C(O)NR₇—;
wherein carbocyclic aryl is phenyl; and
halogen is fluoro, chloro, bromo, or iodo;
or a pharmaceutically acceptable salt, hydrate or solvate thereof.

45. The compound according to claim 44 wherein R₃ is isopropyl; and Y is —C(O)NH—:
or a pharmaceutically acceptable salt, hydrate or solvate thereof.

46. The compound according to claim 1 is:
cis-N-(3-chlorobenzyl)-4-[(4-isopropylquinazolin-2-yl)amino]cyclohexanecarboxamide;
or a pharmaceutically acceptable salt, hydrate or solvate thereof.

47. The compound according to claim 1 wherein R₁ is selected from hydrogen, —CO₂tBu, or —CO₂Bn (Bn is a benzyl group);
R₂ is —N(R₂ₐ)(R_{2b}), wherein R₂ₐ is hydrogen or C₁₋₅ alkyl; R_{2b} is C₁₋₅ alkyl;
R₃ is C₁₋₅ alkyl;
R₄ is —N(R4a )(R4b) wherein R₄ₐ is hydrogen or C₁₋₅ alkyl; R_{4b} is C₁₋₅ alkyl;
L is selected from Formula (V), (VIII), (IX), (XIII), (XVI), or (XVII);
X₁, X₂, X₃ and X₄ are independently selected from the group consisting of hydrogen, halogen, and C₁₋₄ alkyl; provided that at least one substituent selected from the group consisting of X₁, X₂, X₃ and X₄ is not hydrogen; and
Y is a single bond;
or a pharmaceutically acceptable salt, hydrate, or solvate thereof.

48. A pharmaceutical composition comprising a therapeutically effective amount of a compound according to claim 1 in combination with a pharmaceutically acceptable carrier.

49. A method for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from said condition a therapeutically effective amount of a compound according to claim 1 or a pharmaceutical composition according to claim 48.

50. A method for the prophylaxis or treatment of an eating disorder, obesity or an obesity related disorder comprising administering to an individual suffering from said condition a therapeutically effective amount of a compound according to claim 1 or a pharmaceutical composition according to claim 48.

51. A method for the prophylaxis or treatment of anxiety, depression, schizophrenia, addiction, or epilepsy comprising administering to an individual suffering from said condition a therapeutically effective amount of a compound according to claim 1 or a pharmaceutical composition according to claim 48.

52. A compound according to claim 1 for use in a method of treatment of the human or animal body by therapy.

53. A compound according to claim 1 for use in a method of prophylaxis or treatment of an eating disorder, obesity or an obesity related disorder of the human or animal body by therapy.

54. A compound according to claim 1 for use in a method of prophylaxis or treatment of anxiety, depression, schizophrenia, addiction, or epilepsy of the human or animal body by therapy.

55. A compound according to claim 1 for the manufacture of a medicament for use in the prophylaxis or treatment of an eating disorder, obesity or obesity related disorders.

56. A compound according to claim 1 for the manufacture of a medicament for use in the prophylaxis or treatment of anxiety, depression, schizophrenia, addiction, or epilepsy.

57. A method of producing a pharmaceutical composition comprising admixing a compound according claim 1 and a pharmaceutically acceptable carrier.
